SEQUENCE LISTING (111 sequences)

SEQ ID NO: 1 - DNA, 11186 bp, unidentified
  misc_feature 1..11186: note = Draft_10005650_organized_organized
  source 1..11186: mol_type = other DNA
cgtcagccgtcgcgcatccgtctcgctcatgctccaatagtatcctgtcgacctcggtctgtctatactattatgagactattagtaatgtggccgatgctcgccgaccgcggacttgcaccccaagttgccgatgcaacctcgcgccaatatggtcttttctccgacgtcgggacttcgacgcctcccgtgagtgtctgcctgtacgtgaactcacacataactccagttatggcgtgcaaacgttgagcacattgcaggtgactagaaggtgggtgacatttcggttttcacgcgactcgtaacggctcgagcctctgcgaccatcagtcacatcatgcgtcaactgcgcgcaatctcgcgctcttcttagcgatgtggcagtagccgtcttcgcctgactcccgggatacggcggcggccatcgccgttgaactcaagggtgtctcactgccgggtgaggcgtttgactcggacacgagtcacacaagcgcgatgtatccagaagggattgacaacgtggactatctgaaccgctatggctggaacagccaaggttcagcacaccggcgctcaattccctgcgatgtcgcaaagaaacaagcgccacgcacagtaccgacgcgtttgtagttcagggttgttccaggcctcccggcggccttccgctcagcggctagacagtgccgtcctcgtgcctttgagagtgcgcacggagtatctgcctttgatgcgccacacgtgatctgatgcgcctgccgaacaacagatagactgagccgtagaacaccagagccagaatgatgactgctgtcgtggtcaattgcccttacctccgtacgctcatggtagctcgtgtctcaagccggcgccaaaccgtggctgctgccacatggagctatagctggctgagcggaaatgagcgctactggcgaacagaagcgcgaatcggacagcgcgggcagatgtccgagccgccacccggatgcgggtgtcgtgcagcaacagtgtcgcagggccgtgcacgcatgagctgcaccgtgattcgctgtcatgaggcacgaatcgaactcgaacgcgggcgcagacccgatggttaacactcttctctgtggcgcatgacgccagtcacgtagtggctgggccgccgtcggtcagctcgagttgctgtcgcacggcctccctcagtgcgtgcaggcggtcctggctctcgcgacgcttcgcacgttcgaccgcagcgtcagtcttcttcgacttcttccctcgttttccgcgcccctggccggtctccgtgtcctcgtccagcccgtagtactccgtcaggctgactccaataagcagcgcggccgcatcgccgatagtcatgctgtggtagctcgcgaacaggaacagggccgcatgcgttcctatcggcaccttcacctgctttctcacacggtcaaagtgcagtcctgtgattcctggcgtcggtccctccccgacaaaatcgagaatgctgtccctgatgagcttcgctgccgcatcgccgaatgtcatgttgtgtgccgcggcgaacgcgtagagcatcttgtaggcagtctccggtatcttgatttgcttcacatcttcacgcggcctgccccttttcggccgcattatggttctgttctgcatcactgggtctcctcgcgttgatgtttccgtgcgttgacgcgcacttcaccacgatttcgtgtcacattgagtggcatttgttcgtaagtcgacgagttgtcgactctggcattgtagccaatcgcaacgtgaatgtcacatgaaacgcctggatgcacccgcgcggcggtctgcacacacaggaatggccatgagtacatcgatttcggcgcgtgaatgctgcgcaaccgcaccgaaagaactggtagtcgtgacgcagaccagtgaaatccctgtcccgcggtgtggccacggcccacagagggacgtgaaccgccgcagactggcacagaagtgttctactctaagcgaatactgagacagatacgcacgctggtcaccactgtgcacatctcagtgttgttctcgcggcgattagtgtccctactacggtgaacacgaacttccgaccgttggtgagagtcatctatgatgccgtgtgcgggacggactcctgtcgacaccctacagaaccggctgataatcgcatgcgatttcccgtcgcacgtttaacatggcctcgacagacctttcgcgcgtcgggtaccttagccctagccccaggtatccaagcagcgacaaagggtcagtcttcttagaggaagtgtcaggccgagttccaggaccgaaaggttgagggacaagtaaggcaggagtactccggaggctgagtcgaaatagaaactgtgaggacgcttggggctcttctttgtctccgcacgaccgacttccacgagcaccgccgccttgatcgcagtgcagaggttcagggggcggccgttgccatgcaagtacgtgtttgcagcaacgagactctaaacggcactagtatcaacggcgggtaagatcacgccgactcctgagcggaaccctcccttcatagcttgtccgcatctgtggggccacctcactggataccatccggtgtcagcgataatcgggcaatccgttagctgtccgttagctgtggccatgcctccagcaatcacatgaaacaccggggcgcgtcccggaacgcagaccgtgcaacagagcaaaccgagctacctgcccatggagtacaattaggatcgtggtcatgtttctagatagcttctggcggctcggaagagggcagcgggatgaagcgtggcgccgcgtttgccctctggccacgaagcactccgtcagtaacctgaacccggactatcccagttctttgacttgaactctatcgggcaggagatgattgagtgggcaggctgatattccatgaggatggtggagtgactctggatgagcctgaggccggcgccggatatatgccgtacgccgatctccgaaagtccaggcccagcaagtccgcagaatctgagccagttgactcagccgatgccaagccgcgcgtgggcaggcagtcgggcagggaagcacttcccgccgatcaagcgaatacggaagaagcttacgcgtgtcattggccgaggcgctgactgacatcgagtgaagtggctcccctccttctgtcccgttccagagaacaactccacgtagaaccccagtccgaatcactgagtcctcctcagttcagtgtggaaccctccccagagagaagagtctcgttcgccagtccatccggccccacgaccagttcccaagcaacaagttcaaggtcgcgaacagacggttcgcggctgggcggacgctggatgcggcggtcaatgcctggcagttttgagattgcgcagtgcatctagcagctggcccagcggagcattgggcttcaattgatttacgacccgcactgggtctcgcattgcgcgccgcttcaatacatctgagggaagcggctgtcgcatccgttgctccagccaatctcgcagttcggcgtccgccggcaacgccaatccctgcacgcaattgcgctcatgtgtacaggtctagcttacggagatacgacacgccacatggttgaattgtcttgggagtgtgtgcagaccacaatgcgagtctagctggcgctggtatcgctggcggagtgatgtcgttattaggactgcactcgagatcggtaaccgaattcgtagggagtcaaccgagcgctttctcgacaacgagaatccggaggcagttcgcaccgttgacaccgcgagccacttggtctagattggccccaacgccggcgttctcccccacggcgatgaggcgctcctggtatatgcctcaccattgcctcactgccgatccttgcaggcgcgaggaggcatatggacgattcttgcgcttcttcttgtggctgccggagttgtgacagacagttgtcacaacgcagtgtgaccgtagtgccgggagacccgctgttgttggtgacttggataacaaggccttgaacggtccgtggctcgcacggcgccggcacaatgcagactggccgggaggatccgtagcgtgaaatacaacttgcttcaagaaccatggattccggtgcttcgtatggatggctcccagactcatgtcggcatacgtgatgccctggcaagatcggggctgctcttgagtctcggcgagcccagtgcgacggacaatctggctctgcttcgattgctgattgccgtcactctgtggtgcaggtctgaacctggctcctcggagattgaagctctgcgggagtcgcgcggaatccctgaaggctggcttgcacgtctggatgaagactcgggcgcatttgaactctttggagatggcccacggttctatcaagacgggagggtgaagtccgaggtgccgggcagaccggcgtccgaccttctctcatacttcccggcggcaactgaaatcaaccacttcagacacatccacgacgacgatgtcgccatctgcccagcgtgttgtgccttggggctcatacgcctcccggtgcttgccattcagggcggcgctgggaagtcgccgtccatcaataatgcgcctcctgtctacttcatacggcgcggtagaacgctgttagacacactcatgttgaatatgcctcgtggcgggccggctgcggatgacagtcccgtgtggaagcccggcccaaggtcgagcagcgagatcggtacactcgaaggattcacgtggcaaccacgagctgtgtggttgggtccgacgagggcgacctcagatagtcggtgcagcaactgtggaagaagcggccccgtcgtatcaagcctggtattcgccaagggtcaggcaagaaaggatgacacacgctcatggagagacccgcacgttcactacacgaaggagcgcacgggcaacgataacgcagatccagacgcccgcgataacgttctgcgcgcatcgcatccgctcaagtacgcggccttggaggcgtccctctggaggaggacagcctcagcgattatgttgtccgtcgaaccacatgagtgcggcacgttgccagccggcgatagctacgacggaacaggaagcgacgcagtcgttgtcgagtgtattgcgccattcacatcccagggaaaggtcttcgatgagcaccggattgagtggcggataccacgcaaggttctccacgacgatgtcctgcgtcgctctgccttcgaggaactcgattggcttggccggttcaacttgaggaagctcctgggaagggcaagaggtgacggccgcatcaggaagcttgcggctaggtgcctagtcaatcccctcatctgcgagacagagcgccgactgttcggttcattcgtgactctgttgtggtcactggcgcaggagacttcgtcagagcagcgaggctcggtggaacgctggcgacgagaagtacttgacgtgctcgcctcgatgattcagcttgcccttacaacctgtgaccccggatcggctctgaaacgcaggcagatgcaacttcgggcggtggaagcgctccggcatgacgccgggatgactgtcacatcgcgtgggcgcgcttccgtcagacacgagcaagggcaattggagggagcgacaagatgaatccaaccgaggtctacgtgcggcgtcttctggctctaggggacggcgaacgagcagaactccgtcacaaacagggtgcctctctcagtgagtccctggagggatttgatttgttcaccggcctctggtggccattgcgacagcgcagtccggcagcaccgcagagagaaacaagttggctggcggcaaagctgatctgtcagtggccgattccgctcgcttcgtccgatgacagcgtgctacctcggttgctcggcagcgccgagcggcgactgtccgctcgctacgcacttcgtttcctgctgcggttcgataggctcttgcaatccccaatggctttgctggaaccgcagcttcaatgggcattgaaggtggtggcacaggcggtagttgccggttcagccaaagggttgaactgggtgcagcttcttgatgacctcgcggtatgggacggaagacccggcagggaatcaaagcacaatgtgcgcgacgcatgggctcgcgcctatctggcgtctagaaacagggagggaaagcgatgttagtcgagattcacatgattcagaaccacagtccgtcgaacctgaaccgagatgaccttggtgcccccaagagctgcacatttggtggcgtaactcgcgcgagaatttcaagccagtgtctgaagcgcagtatccgcaacccaggaaattctaacgacgtacacaacaaaggggcaagccttttcgtcgaagcgatgcgcgaacacacgggaatcaggacgaagtactttcctgaaatggtgcggcaggcgctgaacgactcaagcgtaccagacgaggaatgggacagagtcgtgcaggcttgcacgagtatcgccaagaaggaggtccagataggcgacgaggatgtacaggagcccggcgacagcgatgagcggcacaaactgccgcagctagtcctgttggagcgtactgaggcaaatgcgtttgttcatgtgttggaggcgatgcgtaccagtgacgagtggagcgccagctaccgccagtggatcgcaggcaaactgcagaagcggcctctcgcagctttcctgcgtgaactctcgcgcgcctattcgagacgatcagtggacatcgccatgttcggacgcatgactacttccgatgcgttcgaggacgtcgaagccgcaatgcaggtagctcacgccatatcaacccatcaggcggtttcggatgtggactacttcacggctgtggacgacctgggcgagttgggcggtggtgcaggccacgttgacgaggccatgttcaattccgcctgcttctacaagtacttcgctctagactgggaccaactcatcagtaacctcggtggctcttcctcttccgcactcgcagcgtccgcactgggccatttcattcgtgcggctgccatgacgacaccaaccggcaagcagaactcattcgcagcccacaaccttccagacgggattctcgtagaagtcaagcggaccggcaaaatccctgtcaactacgccaacgcgtttgtcgagcctgtcgcaatgaatgctccgcggggagtcgtgggtatgagcatcgctcaactgggccaatacgttcatcagattgcagagggttacggcattgactctcgtcgcttctggttctcaccgggggggcgctatcaattgacatggattgagcgctgtcagaaaggagagccgaccgagcgggaagttgttgcaggtgaagattcgcacgcgagattcgacgacttcgtgagtgtggttgtgcaagaggtttctggcctcgaatggaacagggtgaaggatccggacggtttcttgccggaggcgacatagtgactgcctcagacgaaagcatcgtactgctgaggctcgagggccctcttcaatcgtggggtgatggctcacgctttctgttgcggcgcaccatggatgccccaactaagtccggtgtcctcgggctcatctgctgcgcgctcggactgggaaggcgtgaggcatcccggcgcctgcatgaactggcactgctacgtatggctgtacggatcgaccgtcctgggtctcgccaatgggactatcacacagccggagctggatacggcatgctcacagcggagcgaaaggtcaagaagacgcagacgaccggggaatatgagaccttggtgtctcgccgtgagtatctctcggatgccagctttctagtcggcctgcagggtgagagtgcgcttattgaggaggtagcacaggcgctcgggtgtcccaggtggcccacttaccttgggcgcaagccatgcgcaccggcagtgccggtcgtcgcaacgagagtcacagtagtatcgcctggcgccgcggacaacgcccagagtctcctggaggcccttgagacacacccctggcgaccaagacttggcggggtagacttccctccaaacgataacgcgctaccgtgtctattggagtggcgaggcaccatccaaggacagcgacaccaggagcctcttgaagtctggtacgactcgccgctttccttctttccccccgcatatgcaccacggcttgtgatgagaaccaagctgcacgtgtcagtcgaagaacctctgcagcagtatctgtccgctcttccacggccgcgagcagactacaagaacagcgcctatcgcagggcgcgctccctcagattagccagtgactctggcctctgcatcttctgcaaggctcctgctaccaccgtgcagcacattacctatcggcgcgcaggcggcaacgagaccgtggatgatctccgttcgctctgtcgcctgtgccacgacgcagtaacgatgatcgagtacggactgggcatgggactggatcgcatcaatcctgaggacccgcagtggcgcgaggccatcattcagaagcggcaagaactcatccggttccggtcgctggagaccaggcggcgtcatctatctccagaggaggtggagtgaccatgtatctgtcggctcttctcgtcgacacaggcataaaccctgacagaccgcgcccgggccgactctggatgagacaaccctatcgtatccatcagaggttgtgcatgggattcccgctggcggaagcaaaggcaggcgatccggactttgtcagaccctatcgagtgggcgcctttagcggggtgcatcagaagatccggacaagcgagcaagcctttctattccgggtggaccctctggtgaacggcagggccatgattgttgtccagtcggcacgacggccggattgggagtacgccttccgtaatgcggactacctgctggctgggccacccgaagtgaaggaggtccaccatgactatcaagaaggccagtcgctgctcttccgcctgcgtgccaaccccactagacggaacaacgagacccgaaagcgggagggcgtcctaacggaggaaaagcagcgcgaatggctggttcggaaaggacagtctggtggatttgaagtactgtccacgacagtgaccgacgagagcttcaagagggcgacccggaaacacctcgaagagaacgaggactccgaatgcatgtcgcatctttcggtccgctacgacggcatcctgcgagtgacaggccctgaagccttacgcgaggctatcatcggcggcatcggcagcgccaaggcttttggattcggactgttgtccgtagcacctgacagaggatagcggatgcgcacgctcttcgaccttccacggttccaggatcgctggagctacgtgtacctcgagacggggcgactcgatgtagatgatctcggcctgtgcttccgccaggacacccgaacaacgcgagtgcccatcgaccagctctcggtgttgatgctcggacccggcactacggtgacacacgctgcggtagcggcgctctcgcggaacaactgtctcctggcgtggacagggcaggatggcacgcgactgtatgccgccagtacaggtggaacacacagcgccaaaaggctcatcaggcaggcgtcgctggtgagcgacgatgaatcccgcctgcatattgcctggcgcatgtaccgtttcagattcgcggaatccgtaccagacgtcgtgagtctggagagcataagagggatggagggcctacgtgtgcgcaaagcatacgccgacgccgcccgcgcatatggcatcgactggaagggcaggcgatacaaccagggcaactgggaagctgcggacccggtcaatcgtgcgctatctgctgcaaacgcctgcttgtacggtgtgtgtcacgccgcgatcctctcggccggatactctccgggactcggtttcatccacacgggcaagatgctcagcttcgtctacgacatagcagacctgtacaagacaactctcacgattccgctggcctttcaaaccgcggccgagatgcccgagagtatcgaacgcggcacgcgaatgcgttgccgtacggcgttccacgaattcagactcatggagagacttctgccagatatcgcggaggtgcttggtgttggtaatgatgctggggagagtgccgacgaacttgaggggcgcattgtcgcgatggctcctggagccggcgactggaacctttcttgggaacccgagtgggcgagtgagggacgagttgtggatgaaggccgtcaagacaacgaaggagaccgggtgggtgctgcagatatggtcagacaggaatccacaggggttctcctatcgtcagcacggcatccaaacgaggcaactggtggacttcgagggtctggcgctggtgacagtctctcctcaaccgcggactgttcaggatagcgacactgaaacccgttgatgacgagtggccaccatgcattccagtcgtggcaagtcagggtgttccccacgcaggtgggggtgaaccgtgggtaagctagggactgtactaggcggcttcgtgttccccacgcaggtgggggtgaaccgagaccgaggcgcagtcgtacaacgtcgaggacgtgttccccacgcaggtgggggtgaaccgcaggacgagtatcgctcccgaggcaggctcccgtgttccccacgcaggtgggggtgaaccgggcatcttcactggcgtagcggctgacgactacgtgttccccacgcaggtgggggtgaaccgtacgagtacacctgtgacaactgcggtgtgaaagtgttccccacgcaggtgggggtgaaccggttgagaagctcgaccagatgaaccgcgaactcgtgttccccacgcaggtgggggtgaaccggacctgtgcaagaagacaaaggtcggcacggagtgttccccacgcaggtgggggtgaaccgctggtaggcaagggtacgctcaccaacttcacgtgttccccacgcaggtgggggtgaaccgatgatggcctctcgccggaagccgattcacctgtgttccccacgcaggtgggggtgaaccgatggcccgagagtcggcgtcagcggcgaatgcgtgttccccacgca

SEQ ID NO: 2 - DNA, 32759 bp, unidentified
  source 1..32759: mol_type = other DNA
  misc_feature 1..32759: note = DSWX01000005_organized
acacgatccgcttgacctcgttttcagcgtcttcgattcgcttggatatttccacctcctgctcccgcgtcagcagcggcacctggcccatctgcttgaggtacatccgcaccgggtcatccaaaatgtccagccggtttttatcgtcgtcttcctccggctccggctgctttacccggtccacctccgcctgatctacaatctccacctcaagattccgaagcttggtgtagatgtcatccagctcctccggtgtcaccaccgaatcaggcagagcgtcattgatatcattataagtcaggtatccctgctcctgcgccaatcgcaccaattccttgaccttttccgccaggtcaatcgtctgcggttgcttgttgggatcctggttttcatccaccgcaacaggcgcagcttcctccatcgccgtcttcgcaatggattttgccgttttcacctcgcttttagccgcggccttggcaacagcctcgccgcctttcttcgtttgcaactgctctgcctttttgctctcagccgtttctttaaccttcaaagagccgggctttttatccttgttttccacttcggcaggcttgaaatccttcttcttcggtgggacgccatctttcggcttggcctgggcagaagcagacgctgccggtttggagcccttggcctgggaaggttgggcgtgggccttcacttccttattggacgataactttcccttgccagccttcgcggctggctttttcacctgaggttttgccatagatgcgttttagaactatcggctacaaattctcgttataataaatagccttatactttaacgcttttctgttgtgtagtcaatctttgcttcaaaatcaaactttgcccacccttgctgccctgttgaaggcgtaaggtaccacgtaaaaaacactacatttccacaaaacacgggcctgtcttccccccctcccctcgatatttccacccggttataatccatttgtaaccttttccgccaaaaatgctcttggtatagtgtttggaacgtaggcggccaaccaactgcatccgccgcctaaacgccactcatatctgtgggcatgaccgaaacggtccataccgaagtgttgccgctccattcgacggcggatttcaccgccgcagtggagaaagccgccgctttgcttcaggctggcgaagttgtagcggtccccaccgagacggtgtacggcctggcagctaatgccctcaacccacaagccgtagccgccatctacacagtcaagggccggccttcccgcaatcccatcatcgttcacgtggccgatgaaagcatggcccgccaatgtgtcgcctcctggcctcccgctgctgccgccttgtcccgtgccttctggcccggacctctgaccatcgtcctgcccaaggcagccatcatccccgacatagtaactgccgggggccccaccgttggcgtccgctggccgcaccatcctttcatgcaagctttgattcaacgttgcgcctttccactcgctgcccccagcgccaacctttccaatcgcgtttctcccaccaccgccacccatgtgcgccaacacttggaaggacgcatcccccttatcgtggacggcggcccctgccccgtcggaattgaatccaccgtggtggcccttaaaggtgatgtttggcgcatcctccggcccggcatcattcacaaggacgctctggccgccgtcttgggcaccggctctgctcatgaggccgcctcacacgatagcaaccgccctgctttggaaagccccggccttctgccgcgccactacgcccctcgcgcacaagtgttggtttgggcctgggaaaacgaagactccctgcttgcgcagattcgccgcatgggggcaaaaccggaggaaacttacgtcttggcgcactcctgtgcgccccgccaaaaggactggcttgaagtcagcgttgcaccgcgtgacccggaagcctatgcacgcgccctttacgcggaattgcaccgggcggatgctcacaatgcacggtggctggtggtggaacagctccccaccgatggcccatggcaggccatccatgaccgcctccgccgcgccgctcaacatgaagaaacttgaattttacgtcggttgttgattgccatgcctcaaccgcgttacgatgaattgacgttttgatgaaaaacttgcaagctcgaccaatgacacgactgggaatcatcgcaagcctggtggccgtcctcggcgccacggcctggggtcagaccaaccatctgacggaacgcgccatttctttggatgaatgtctggaaatggcattggaaaacaatttcacccttcgcattgagcgccacaacatcgagcaagcccgcttcgacctcaggcgctctcaaacagcctacgacccgcagcttttctttggcctcacccgggccagaaacaccggcctcaaccaaaacttcgatttattgggcaatcgctccgagaactcctcccaaaacacggtggacaccctcgaatacggagtcagcgggctggggcccatggggtttgaatactcggtgccagtgagattcacccgcagcttcggcgacggctacacgtacaacaccctcgatctcaacaccttcaccttcgtgccggtgggttacgacgagtacgctgcccgcttcaacatcgaaatgcgtcagccgttgctccgcaacttcgctgtggacaacgtccgccgcaccatccaggtcaaccgcaaactgctcaaaatgtccgagcaccagtacgaagcccgcctgatggaaatcatcaccttggtggaactggcatactatgaactgatcttcgcccgcgaaaacgtcaaagtccaggagaaggccctggaactggccgaaaaactcctcgataacaaccgcaagagtttcaatgtcggaagcatggccattctggaagtgcaccgcgccgaatctttggtcgccagcacccgcgccgacctcctggccgcccagcaattggtcaaagtgcaggaaaacgtgctgaaaaacctcatcaccaaagacctggccgagtggaaacaagtttccttgagacccaccgaatccctgatggccattccgcaagccctcgacgtgcgcgaaagctggcgcaccggtttgaccaaacgccccgacctcgcccagttgcgggtggacttggaacgccaggacattgacatccgttacttcaaaaaccagaccctccccaacctgaacctcgtcggttcttacggacgggatgccctcggaacgggctttgaaaacacttggtcagacctccatttccgcgacaaagccgtggactactattacggcgttgaactcagcttcccatggggccgtcgcgaggccaaataccgtctggcgcaggaaaaagaccagcgcctgcaaaaggaattagtcctccaaaaactccagcaggacatcctggtccaaattgacgatgccatcgaagccgttaaaaccagttatgaccgcatcgccgccacacgccaagccgctgattttgcccaaaaagtcctccaatccgaggaatccctcatgcgcgtcggcatgagcacctccttccaaatcctgcaataccagcgcgacctcaccgcagcccgcacccaggaagcccgcgccatcgcggattacaacaaggcattggctcgcctagcccaggcggaaggctccaccttggaacggcgcaaaatcaaattacaattcaaataaaaccgccccggaaacgcgaaggttaaaagcctctcttgcagtggctgctgctacctgctgccaattcgaaaccaaaagcactgcgggaaaacttcccgcaattacaccagcccctccaaaatcatcggcaaccgctccaagctgcggttaagccctgcccgcgcaatggccttggcactgtcggtttggttggggaacaacaaatgccatgccggcgtttcaaaccacgcttccgccgccacgctctggtaattcccgctggtcccgatttgggcatgccgcaacagcagatccgccacatgcaccgccgccacaatttgaatatgctggctggcgcgctcgggcgcatggtgaaaccgggccgactccaccatcacctgcggcagtcggtgttgctccaaatacaacccgcccaactccgcgtgatccatccccagcacctcaatttcaatcgcctgcaacggcgcttttgtctcggatgaacgccggtgaatctcggcaaaatgctgcggaaacaccgcggccatcacaattttgcccacatcgtgcaccaatcccgccacgtaatccgcctcgtcattgggcattcgcaccgccgacaacacttcccgcgccataatcgccgtgccaatgcaatgacgccaaaactcccgccatggaaattccgtgttgcccgccatcttctgaaaatcctcgatcaccggcgtcaccatcgccagttgccgtatttgcctcagccccagataaaaaaccgcctcctcaatgctgttgaccggcacattcagcccaaaataaaccgagttcaccaaccgcaacaaccgcgccgtcaaactagggtcccgccgaatcacctcgctgatttgcgccgtataactctgatcagccgccaacaactcccgcagcgcactgttgacagtgctcagcgatggcagacgcgggcaggaatgtaaccgcgccgtgatctgttgccggttcagcggcgtcagccggttcgtagtggccacttgactcatactcatcttccctatcggcagtcagaggaaccaactttaataaataataaaaaaagacctctggttaccctcccagcgtccgatttttagacctccacaatcaaacagccttgggtagatacacagaagccgcgcttgacggcctgccctgctttggttaccaaagcatcaccaatacagcacaatacattcgcgggccgggccggtttggcgtccctccccgccttggaaaatcacatgaatactgagaaaaaaggcctgtttgtcaccgccgacggcagaaacgtcgccttcacctttgcgctggtcagctcgttgtttctcctctggggattctgcaacgggatgattgacgtgatggacaaacatttccaaaacgagctcaaactcaccctcagccaatccgcctgggtccagtttgcccattacctcggctatttcatcatggccctgccggccggctggctggccatccggctcggttacaaaggcggcattatcgcaggcctcttgatggtggccgtagggggattgtggttcatcccggccacacacatcgccaagttctgggcctttttacttggcgtctgcaccatcgccgccggtctgaccttcctcgaaacggtggccaacccatataccaccgtcctcggcgacaaacgcttcgcagccactcgcatcaacctggctcagtcctgcaacggcgtcggctggatccttggccccatcgccggcggcctcttcttttacggtaccgatgaaacaggacgcagcaccggtgccgaaaccttgtggatcccttatgccggtgtggccgtggtggtcatcctcctggcggtgattttctttttcgcccccatgcccgacatcaataccgaggacgactatcacctcgacgataaaaacaatgacaaaaccaccgccattaaaccggcttcccgtgggctgacctacgggttgttactcgtgaatatcgccatcctgcttttggtgtttggaatgataacatggctcattctcagcgcgaccaaagttggcgaatccctcaaaggcctggccgctgccctgccgcatccttcctccttcaccatcaacgcagaccacgccttgaccgtcgtcctgttttgggcgggcgtcgccatcatgatcgtagcggccattgtcatggcccccatgaccaaaaaaatctcccaccacagcatctggtcccacccccatttctccggggccgtggcggcgcagtttctatacgtggccgcccaggcgggcatcttcagcttccttatcaattacgtgacctcagaagtcccggacctccccgcctcgtggcaccgcgacagcaccaaaaaatggattgaaatcaacacctccttcacacgcgatgacctcaaggatttgcctgcgctggcggctcgattgaaaaaccccaccgaccccgtgacagccatggtggccggcaaattgtcctccgacactcaggcagccctcgcagagtacgtccccgacaaaaccaaccccaaattgctcagtgtgcccttggtgcaggatttgaaaagcattgtccgcggcaccaacatctacaatccccagctcttcgctggtgtcaacctcagccccgccacccgcgaaatcctggccctccctgaaaaagaacgaaacacacccctcctcaaccgcctgctccttacagacgcctttgccggtttacttaattataccccaggccgttattgcatcagtgacgccggcgcttccaacctcgcctctttcggattcttctgcttcttggtgggccgcttcaccggagcctggctgctgaaacgtttctccgcccaccgcatgttgggcctctacggcctcatgaacgtgctggtctgcctgctggtgttcttcaagctcggctggctgtccgtggtgtgcgtcttcctcagctatttcttcatgtccatcatgttccccaccatctttgcactggggattttcggcttgggcgtccgtgccaagcgcgcctcggcttacatcgtcatggccatcatgggcggtgccatcctgcccaaactcatggggtacgttgctgaccaaaccaacatgtcccgcggtttcatcgtgccgatgatttgcttcgtggccattgccctttacggcttcggttggagccgcctcagccaggccgaatccttaagcggcctgaaaaccaccgccggccactaaaaacaaggctcccattcttgcaccacccccctgcgctggggccggggctcgtcttgtcttcaaccaatccgcgctcacactttctccggcaccacaaacggcggacggtactcgcgccgaagcagccgatccgcctccggattgttcacaaacttttccttgtcggcatcaaacatcaacttgggccccacccacaacgtcgccttctgcgggtccacaccaatcgccttggtgttttccaaagtcgtcatgatgctctcgcccaccacctcgttatcgctgaaatccttcggcttctcaaacggtcgctccttggccaaccggtaggatatattgcccaaatgacagcacgcgctcgaaaggtgcccctccaaaatgtctgcgtgcagattttcccgcttgcggctgcgcacgcactcgatgaaattggcgaaaatattacccgagcgcgacggcgcatccaccttggccaacggctccccttcggttttgtctttggggaaaaacttcccatccttcaccacaccggcctcaaagaaatattcattggcaactttgttgggatactccttgagcttgcccacaagcccccgcgtctcaaacaacagcaatgtcccaccaaaatcgagcaccgtcaccatctgattcggcgtctgcccttggtccttgtgatccggcccctcaacgtaccgcccgcccaaactgatgaccgatcgcggcaacgtcccaccaatcacccaccgcgccacatccatctcatgcacaccctggttgccaatgtccccgttgccgaagtcccaaaaccaatgccagttgtaatgcaccaaattccggtgaaacggctgtttgggcgcaggccccagccagatatcaaaatccaaattggctggcggttcctccacaggcttgaaaccaatgctccatcgtggcttgcaacaatatcctttggccaccaacaacttcccgtaaacaccgcttttggccaacgccaccatccgcacgtccccaccggcgcgccgctgcgtcccgtgctgcacaatccgattgtacttccgggccgcctccacacatttgcgcccctcaaacacattgtgactgcacggcttctccacatacacatccttgcccgcctgacaggcccaaatcgtggccagcgaatgccaatgattcgtcgtggcaatggagactacgtcgatttccttgttctccaacatcttgcggatgtcctgataagtctgcaccccctgcaacttgctcgcacgtccctccaacaccttcaaatccacatcgcacaacgccgtcactctgactcccggcaatttctgaaactcgctgatatgattggccccacgcccgtttattcccaccaccgccacacgtatggcctcattggcacccacggattgcgcatacacacgcggcgccagccagatgccggcggcccccaaagttgcggttttcaaaaaagcacgacgcttcaacgacgaagactgcatgtaggctttcatgcccccactctaatctctccgcgccccgcttcaatcccaaactccaactcccaccaaacaccgactaattatagatgaatggcagtgcaggtcttcccaacataagatgagcttgcgcttgcccttccacgccgcggccttacattgagcgtgtgcatgatgcttcggatgattggcgccggcgttttgaacgcctgcaacagttataccacgtcagccaggtgctccattccactctcgaacctcagcgagccctggaactgattgtggaacaagccgtgcagctcaccggcgccaccagtggttcgctggttctcctgaatcctcataccggttttttggaaattgaagcctctcacggccttccacccgaggctcaaaaactcaagctccgcgccggcgaaggcatcaccggctgggtcgtccgcaccggccagccggcactcgtcggcgatgtccgcaaagacccccgctacatcatggcacggcccggcgtccgctccgaattggccgtccccctccaagtcggcgaagaaattcgcggcctcttgaatgtggactccgacaaacttaacgccttcaccgccgaagaccaatccctgctggaagaactctccgctcaagccgctctggtcatccgccaaacctggcaatacgaacaacaccggttgaaagcccggctgtttgaaagcctcgtcgccgtcagccaaatcatcaattccaccgtcaatctggacgaagccctcgccgccatcacccaacaggcctgccaactcatgcaggcacgcctcagctccttgctcctgctcgatgactccggacgctggctggacctccgcgctgcgcacggcgccggccccgactatatcaacaaaccgcgcctggcagtcgaggacagcctggtgggctccgttgtgcgccgccgcaaagtgcttcaggttgagaacgtccaaatctcccagcgctatcaacacgtcgaagtcgccgcccgcgagggtctggtctcgctgctcagcgcacccctcttgtttgaccaacgttgcatcggcgtcctcaacgtctacaccggccggccccacgtcttttccaacgaggaaatccgcatcctcaacgccctggcgggtttatcggccgtcgccatcgaaaaagcccgcctctacgaacgggtggtggacctcgaagaagccctccgccaagctgaaaaattcagcgcactcggcctcttggccggcgaagtcgcccacgaaatccgcaaccctctcaccgtcatgaaaatgctgtttcattccctgaacctgcactttcctctgatagacccgcgcaataccgacgtgcgcatcatcgccgacaaaatggaacaattggaccgcaccgtcgaacgcgtcctcagcttcgcccgcacagccgagccccagtttggccccgtgcacctccaacacgtgctcggcgagctggaactgctggtccgccacaaattgcgccagcaaaacatcctttggaaactcagcctcgaacccgccctccccgccgtttggggcgatgccacccaactggggcaagctctcctgaatcttgtgctcaatgccgtccaagccatgccccaaggcggagaattacgcatctccgcccgccgcctgccgcgcgaccccacccgccccgccacccatgtagtcttggaagtagccgacacaggcgaaggcatgagccccgaacaatgccgccgcgcctttacccccctcctcagcaccacccgcaccaagggcaccggccttggactggtcttggtgggccgcatcattgaagctcaccacggacgcatccaagtcacctcccgccccaaggcaggcacacgcttccgcatcacgctccccgttgaaaaccaaaatcctgcgtaattccccccaatcgcctccggcggcccccttgacatcaagttgaacgtgggacaagttgccattgttgtattatgaattttgcgtggcttagcctggaccttgttttgattgtgctgtttgccttttcgctaccggcacttttcctatatgtcatccgccggtggctccaaaacagcgaatcggcaaacccatcggtggacaaccaaacaccggacccaggcaaccattcatctcactgaatgaccaacggattttttctcgccttcttcgcggcttcctcaaaacaggctttctaatatgcttaagccctaaggaggctcaagccatagggttgtatgccgctcactctttgctcgggaaaaaacagctccaccgattatttcaccattagatctcccttgggcactgaaaaactttttcgctctatttttgctgtaaccgccttttttgatttgttctgaccccgcggctgggcttatcttgaagcagtatagcgatggaccctgcccactcaacgtccagccgcaaccatcgggccacgccacacgcgtggaggaggtgttcatgttgacctttgaccccacgcgattgcaagcggccttgcggttggctgcttccgagctggtcaaagctcgtccggcaggttctccctggcagggtgaaatttccagcagccccacagccaccgctgccgccatctgcgccctcagcctggcggataccgagtggaaaagcgccgaggacaccctgcgccggctcgcgttacatggggcggagtggttgagccaccacgccaacggcgacggcggatggggcgacaccccgcgcagcttcagcaacctcggcgcaactgccctcgcttgggcggccttcgggctggcaccgggaggcgaagcagcctttcaggaacccgcccgtgccgcagagcgctggatatgccaaaaagcgggcaataccgcccccgccatcttggcccccatgattcgcacccgcttccgcggggatttggcgctggccggtggcgttttgatgtgctgcgcactggcgggacgactcggcagtgacattcgcgcctggcgccacgtcccctccctgccttttgagcgtggagcactccctcccgaatggtgggcgtcagtccaaccgcccgctgccagttgggccatgcccctcctcctcgccacaggccaggctcgccatcacaatctcgcaggcggctgtttcgtggtccgactgctgcgcgaattctttcgcaataccactctgcgccgcctcacggccttgcaagcagaggacgggaattttcaaacctcccccctcttaactgccctgaccaccatcggcttgctcgccgccggctacggtcgccatccagccacagaaaaaggcatccgcttcctggtaacctccgcacgtcccgacggcagttggccgctgattacaagccaggacgtccgccttaccgcgcaagccacgcaagcctgcaccaaccccgtccccgggcagccgtggctgccgggcctcgacgaacttcatgccttgcgccattggcttctcgcccatccgcaacacgatgacccggagggactccgcctcccagccatcggttggtcggccagctcacaccccggcgcgtggcccgacaccgtttccaccgcagaagccctcttggccttgcgctcgctcggtgacccgggccctgaggtcattcaagccgtcctcggcggcgtcaattggttgctgttgacccaaaaccgcgatggcgggctgcccccttatcgtcgcggctggtgccacccggaacaagaaggcagttgcgccgaggaaaccgcgctgaccttgcgggcttggctggcctggttgcacgtccttcccccggaccaacaacaccgtgttcaaacctccatccaccacgccatccgttttctcatggacgagcaacgtcaagatggcgcctggcgttcacactggtttggcaatcaatacgcgcatcaggaggaaaacctcaccatgggcaccgccggagtcctcatggccctgcccgaactggtggcccgctcctatttcgagttggtcgaaccactcaacagcgccgtcctctggctcgtcaaaaaccaaaacagcgacggcagttggggcggtggccacggcggccctccctcagtcgaagaaactgctctggcactcgaagccctggccgaaacactcctccaacctggccccctcttgctggaaactcgtgaacaagcacgcaacgccgccgcctggggcgccaactggctagtccaacatatcgaagccggcaattggaccacacctgcccctctgggcttgctctggggacaattctggttctacgaaaacctctggccggccctcggcgtcacccgcgccctggcccgtttgacacgcctggtttaatcccgccaccccccggcgttgtcatccgcggcaggcggctcatgtgccgaaaaaccatccgcgacaatggcttcttcggcgaataatgcaccatcatgtcctgcatccgcatcaacacccacggctcccgcggcgcataaccgtttttggtgaacacaatgtcatcagccacgtacacacacatgtgcacagtccggacgtcctgccccatcggctcatacaacaggatgagatccccaaacaaatccgccttgggcacaatccgatactcgctccgcaacacctgctcctgaaatcgtgcatcaatgaaccggtcctccaccttgtcccggtgaaaattatacgacgaccaaaaacaatccggccacctccccgcaataatatttgtgggatgcggatacgaataaagcaacagccgcggcaccggcggaaaaaaccatgccacattcacactccccccctgcggcaggcgcttcaatgactccaacaacggccgcacctgaagcattcggttgggcagcccccaatagcccatcagccgctccacatcgctggtgggagtcacccgcaaattcaccataagcgtcggcactcgtgccaccgtccggctggcttgcagataatcctctctcgttaacagttttggcaggacatgccggtccgcaaaacacagcaaaccatctttccggtacatcatccgctccatgagcttcagcgcatcttcagacaaactgcattgcgaaaaccattcggaaaactcagcattaaccgcaaacggataacaataagccacattttgttcatcctcggccaaaatggagtagatcttggcccgcggctccggcggcatgtcccgcagcacttcaactgtcggctccacataaactcctcctgtcgtcgcctgccaacggttcgtgtccaacaagctcgcctccaacaaactcggcaacccgcaactttttatgaaatccctcaaagtctccggcttataaccggcaaaaaaccacctgttcgaggaagggggaggcggatcgtacgcaaaatattccactggccgctccaaccaaattgggatgatttccaatattccccagcggtttgttgaggcttcatccttcgccaccaaaccgttgttggatgagcttcgatcagagcgcgaccataaagtgcgaccaataaattgcgcacccaacgtaatcaatacccctgcaagaaccagccataaacttgggagaaattgccggcgaggcacggccccattctgccgctcgctccgtgatgaagtcttggctgggcgcattttcataaacagcctcccttaccactcaaacagcgcccaaagaccagccggcccaaaaacacccctctcccgtcgaagtgtttgcctgttattcgccggatgtattaatcgcccgcccttaaggacgttattcccttgcagccatcccattcaaataaagagacgggacattacctgcgaaaggttacaaggacaacggttttgtgaaaaaaattagcctgaaaaccctcacattgtgaacttacctaccagacggtaagttccgccagggacgcaaagggttgacggtcaaatccgctcacagcctccagcttcaaataacggacctgtaccggtgccggaaacgtcaattcctttggattggcatcggcggacaatgtgccccgcatcaaaggtgttccccagttttgtccgtcgctgctcgtgtaaatggcaaactcgcgtatccacccatttttattgttgtcctgcctcggcgtcagagtcacaccacgcagagtggccgattgttgaaactccaaaaccagccaatgcggaaaagccggagcttcctccccccatggcgtgtgccaaagcgtttgcacgtccccatccacgaccaacttgcccggatgctgcggatgttcactgctcacccgcgatacccggacccccagtttttccatcaaggaaggcggcctcaacaacttttgcacatccgccggcgtccaccgatgcgcgggtttgaaacgtggaccgctcatgtaattcaaaagactgcgccgaagctgccttgccaccggacgctgctccaagtctgtattcaaatccatcgcacacgccaacaaacgcccccgtcccacttgcgcctcgatgattaaccccagtttgcgcgacgtaaaccaatcgtctatcgcatacaccaacggccggaatgtcgcctccgtgcccgtcaacacaaacggatgcgcccgcgacaccaactcccaccattgccagttggaatgatattcagtcggaaaatccgccagcgccggatgcctcggatcgcaataaatgcccaaagtatgcggcgcctgacgccgcgtccacgctgtgttccaaaagatgctcgaaaaaccaatttgcacccgccccaacgcatccccgcgaatggccgatggcggcggattcaacaacaccttgcctccgcgctccaaaaccgccaaggccgccgaatccaaatcagtcgcccaatgaatatcctgcggcacactcacatccaccttggccggatacacccacacatcccaatcattcgcataaggcgtgccttccaccgccagctccaaattcaacttcgcaggcgcaggcatccccttcaatggcacctcaatctgccccaccggactgcagattccagtcggcaacggaccagtgaacatcgtccccgtcgccaactcacgcttcccctgccgcaaccgccagcgcaccacaccctgcggcaaatcctgcggtgcaaaccatcccaactccaccccggcctgcagcgtgtcctccgtggtgaaaatccgtctcggcaatcgcgcaatcggcaccaccggcccggcaaaccggtgatactccgcagccgtgatatagcccttgccatcccaaaacggatccaacacccccaccaacgccgtcccctgcccaggaaaatcatgcaaatccaacaactcgaatccccccatccccggcgtgcgtatcgccgactcaatatcctccttgtaacaaagcgcctgcaatttccccgacgccagcaaaaacgcccgcgccataggcgccaaaccgttctgcttcaaaaagtcctgaaaaatctcaaaattcttcggtttcagccagcccttgtatttcggcatctccgcaaagttgggaaacacacaccattgcccaatctcatgactcaccaccggcgccgcagcccgctggataaacgcccggtaatctgtcaccgtctcaggcggacgcgcattgatgcgtgaattcaatcccccgccccatgcctgaatgcgcggctccggtgtgatgtgaaaatcattttccggaatcatcggccaacccgccccgctggtgtaaagccgccgcgtatccaggcccttgcaataattgatgaaatcccccagccaccgattctgattctttcccgccggttcgttgccggctgaaaacatgataaacgacggatgattgcccaacacccgaaagatgcgcgctgcctcctcgtgcaaccacgcatccaccttgcgcccatcccccacactgctccccgaattcgcccaaacaccggcctccacatacaacaacatccccgtctcatccgccgcctgaaacgccgcctccgggggagtatgcgaatgaaatcgcaggtgattcaacccatgcgcctggcaaatccgcataatgcgcttccacgccgccacatccgtcggaggatagcccgtcaaagggaaaatgttgcactcaagagtgccacgcagaaaaagtttgcggccgttcaaagcaatttgcgtcccctgccgcttcacctcccgcaatccaaacgacacctcccgttgatgacgcaatacccgtccttcatccctcccctccaacgcagccgtcaattcatacaaaaccggcgaaaattcatcccacaacaacgcatcctcttccatctcatactcacactccaccaccccaccctccggagtccacgaaaccggcacagtcaccggcggcacccgatgctgccgtggtccattgtaacttcgcgccgtcaacgtcaacgtcccactgccggagcgccctgtgagattgcccaaccgcgccttgataaccgcacggcggcgagccacgtccggatacacctgcaaatcgtccaaccacacagggtccgtggccgttaattccaacacccccacaatcccattccagttgccttgcgtgtgatccgaaacactgtgcgcattgatccccacctccacatgaagccggttgtccacacgtaccgtcagccgatgccgcccaggcgtcaacgccgcagtacaatcatattcatgcggcgtccccaaactgttccgcgacccaatccggcgctcatccacccacacagtagtctcccaatgcggccgctccaaccgcaacaccacccttttgccctgccacgacgcgggaatctccacctcccgctgataccacgccacccccacataatgccgctccggttgcaaccaaaacggcaccttgatattgcccggttgccggtagggcgcataacgctcatccttgaaaaaggatcggtctatgatttggcctgtccactgcgtctccatggtcaccgggtccccaaatccctgctcctgcaatgaaccaggcaaccgcagccgctcctttaacacccggccatgccacccctcccccacaccggcatcctgccggtccagctcaaaccgccatgtccccgccaaattgatgcgctccgcagctcctgcgtaaaagttggccaatgcaaaaacaatgcatccaaaaaatccaactgccagcctttgggcgcccttgagattgaggtgttgggtcatcatgttgcaagggcagtattgcaaatgccgcactccagtttcaacacccaacaatcacccctctccaacggtggacaaaaccaaacaactcggaaatacttccaccatgaaacatacccttgcctgcatcgcactcctggcttggctggccacggccctgcccgccgccgaacgtttccccaacctcgcagccggtagcaacctcccccaaggccccggctttgcaagcaagttcaaagcagactccggcatcgccaaccatccctccgtcatcttttccgatgattttgaacagggcgatttaggggcacgctgggacgaaaaaggcgccggcaaaggcaaggccctaagcctcgtcccaccaaacgatacacgttgtggccgccgctgcatgaaagtcgaagcccgactcggcgaaaaccagggcgacggcctcaccaaatggttcaaccccgtggacaccgtgtacatccgcttctacgtcaaatttgaccccaactgcgactatgttcaccacttcgtcacacttcgcgccaaccgcggcctccgcggcgcagaccgctggagcggctttggcggcgcaggactcaaaccacgcggtgacgagcgcttttccacagccttggaaccttggggcaattgggccaagtggccgcccccgggcaaatggaacttctacagctattggcacgaaatggatgtctcccgtgatggcaaatattggggcaactccttccaagcagaaccccaggatgtcattcccaaagaccgctggatttgcgccgagttcatgctcaaacacaacacacccggcaaacccgacggcgaacaagccttttggattgatggacgcctcctcggacattggaaaggcatcaactggcgcaccaacccctccctccaagccaatgccctgaccttggaaagttacgtcaccgaccgctggacaaaaaaccccaccaacaccgtctatttcgacaacctcgtcatcgcccgcgaatacatcgggccggccaccgccccctaaatgggcagcgtgggctgcggatttgccgtccctttgtccggcactgcggctgccgcagcttgcgcagcgccattctcaacctgcaccaacgccggccgcaacaccttgccctgatacgtatatcccggcgccagcgtcaaaaggatgcgcgtgcccgcagccggtggctccgcacactgatgggcctgatgcatctgcggatcaaacccctcgcccgcctccggagaaaacgcttgcagccccacccgccgcgccaaatcacgacaaacattttggaacaacccgatttgctccaccaaatgcggctgcccgctctgccgcgccgccatgtacaacgcaaacacctggtccagcagacgcaacgtcaactgcagccaatcctgctcctgccgccgcaacttgtccgcttccaaccgcaacgtcgccttctcggtatcattggcctgccgcagaaaatccgtgaatgccttggcctcagccgccatcccgtcggccacttccttcgccgtttgcaccgtccgcgcacattcgccctggatgttcatccacgcgcccgtagcctcggctatctgctgcttgatggcctccaaattttgaatctgcgccactgtcgaacgcaacgcatccgcctccgccagcttcatctcagcccggtgctggagaataaacggcgtcaccaacaaccacgccccggcagccaccgccgccgtgcacagcgccatattcgccgtgctcaacggccactcgctttggtacacaatgccaccggccaccgctaataaaagcaggtcgcccaataaaaacggcaacaccgacggtctgggagtcgcaacgtcactcatgcgcccgccagcttatgtccttaggcacaacccatgcaacagcgttttaccccacccacaacatcgccactccttttgtccaaatcaaagaacccccaccccaaagcaccccgcccctctcacaccccctgccccactccttttccccgcaaaatatggccattccttgccaaagccggtgtagcaaacaaccgccggtattcataagtttttcctgccatgctgagacaccagcctgtgcgtgttttatgaaaacagtccccttccccagaggaaggggcgaataaccggcataccgccacaaccaacgcacaggcccggaccccggctggccatgcaaatgagcggaagtcattccatgggcgcgcccacggccgcagccaacgtccccgaccttcaggggaacgggatcgaaggcttggcaagccccttgccggccatgtccagcgcctacgacttcctccaaaaccaatacgtttacgtcatcgtctccgcccgcgcccgcggattatccgttggcgtcaacctgaaccccgaacgccgctgcaacttcgattgcctctactgcgaagtcccacgccccatggccggtggaaacccgccggtcaacgtcgaagtcatggcccgcgaagtcgagcgcaccctcgcctacgtccaatccaaccgcctgcagcagcaccccgtcctcggcacgctcccacgcgaatttgtccaattccgccaccttgccctcagcgggttgggagaacccaccctgtgccccaactttgtcgaagtcgtccaaacactcgtccacctccgcgctttagggcggctgccctacttccacctcgtcctcgtcaccaacggcagccagctcgaccaccccagcgtccaggctggccttaaatactttaccgccaccgacgaaatctggatcaaactcgacgccggcacacccgagtacttcctcaaaatcaaccgcacacccgtcccctacgaaaaaatcctcaacaacatactcctcgtaggccgacagcggcccgtcgtcattcaaagcctcttcgtctccgtggaaggcgaggaaccgcccgcccgccaaattgagcagtacgccatgcgcctcaaacaactcaaacaagacggcgcccaaatttccctcgtccaagtctattccgccacacgccccacccaccaacccgcctgcagccacctccccttgaaatccctcagccgcatcgcccaaatcgtccgctgcaccaccgaactccccgtagaagtcttctaacccccccgacctccgtcccacttgacagcccgtggcaaactcctacgttgccaaccatgcatcgcccttccgcttcatcctttggcatcgggcttattctggggttgctcctgaatgccccggcacccaccttcgccgcgttggatactcgcgagctgcacacagaagtcaaaatcaccattcaactgaaatacctcatctggctgccggaaggttatgccttgccccaaaaccgctctactcaatacccctgcatccttttccttcatggtgcagaaaaacgcggcacaaacataaacgctttgcaggaccttggaccgctgaaatatgcgcgcacacatcctcaagccttcccttttatagtagtagcccctttgtgtcccccgggacgccaatgggagccgttggcgttactgacattgttggatgaaatccagcgccagtatcgcatcaaccaacacgccatttacctcacaggctacagcatgggcggtgccggcacttgggccactgccatggcggctccagaccgctttgccgccatcgcgccattgtgcggcagggtcattccactgcttgcaggcaacttgtaccgcacgcccgtctgggtctttcatggtgaaaaagaccaggtcgtgcccctgaaccactccaccgaaatggtggacatccttaagaatcgcaataacccccacgttaaattttcactgctgccgggaaaagaccatgaaatatggcacgatatttacgataactccgccctctactcctggttcatggaacaccgccgcccctgacttgagtacaaccccacgccgcgatccatctaggtgaggcaaaaaacaattccccgcgcaacaacacctttgcctccggagacacgccttcacaatccccgctgctccttcctctgcccataccggccaccatgccgaaaagcaaaattcgccccccttataaattttccatttttcttttgacaaccaaacttttcggcctagagtgacctcatagcttggcaaaccacgtggattaacttcgaggtttcccaacatttaaaatttaaaaggcctgcttatgaacgactttaacctgatagaggagccgtggattcctgttatttaccacaatggcaactattgccgtgtcggcatcaagcaggctctttccgatgcccatctcatccgcgacatcgccacctccaaccccatggaccgcgcgggccttctacgctttctcctctctatagtttattggtgccatggaatgccagcccctcaagaaacgtctcggctaggtgatcctttaccggattgcacaccacatcttggtgcaaagacaaattttttctgccttttcgggcagaaacaccgtttttaccaatcaccaacggcaaaccgagaagtaaaaattggtgaactgtttcaagaaattccatccggaaacaatttctggcattttcgacatgtcaccgataaagaacatggatattgcccagcctgttgtgctctaggcctgctgcgccttcctcttttttcagtcagtggattgccagacttgaaatcggggattaatggaactccacccatctatgtctttcattatggcaaatctttagctcaaattctccgaaaaaactggttgccaatgaagcaaaacttgctgggcacgccatgttggatggaccccaccttggttccaccaatcggagaccctatcccgctgttaatcggattaaccgtcccagcacgcaaagtttggctcggacaacccattcaccatggccgttgctctttgtgcgggaacccatcaaaacacctggttttcgaggctcacattcaatccgccggtacgttggaaaattcaaattggcaagacccacatgttgtttatttcctaaaccaacaacaacgaaagtcatgcaaagctcccaatctcgtcgtaggcggtgaatttcaaatggaccgcccttatgccgatcttatggcatgtctcttggaacgggtccaatttcccgcaactgcgacaagccaacttctaattgtcggttttgccacggacaaagctaaaaatatagatgtttgggagagaactgtaactttggtccttaacgaaatgcagacaaccgcagccacagctttttccacctggtggcgaagtgtaagtgcattgtccaacaatgtttacactattattaaaacaaccccctacaatcggcctcgtgggtccaaaccctcgtatgttcactgccatttttcccacatcagaccccatgtggaacatataatatcatctaacatgcatattctagtaagtgaaccaaacgaaggttggcatgaagcacgccgtgagttcagcgcttttttgccgatgttggctcaatccctctctcccggcgtgacctcccaagccgttcaaaagcgccgtgccatcgctcaggccttgccacctaatccctgcccgaaaccggaaccatccagttcaccctccactcaaaaccccaacttgtaattatgagcgccacccttgaatacattacaaaactgtcccatataaaggccggctcccgcactttggttcgcactcatgccaatctctcattgaaccattccgtggatgcctttgatttgttcaccggtctttggtggcccttgcgccaacaatcacaatttgcaccccgtcgccgcgtggcctggctggtcgccaaacttcacgcagtcagtcctctgccacaccaaccaggccagaccctccccatccagctcgcgaagatttggcataggcagccagacaccaatacaaaaaacaggttccagcgcaaattcgatgatctcctttgccgtcccctggaccaaatggaacccgctttgcaatgggccttgagaacaattggctctgcaaatgagccatccttggactgggtgcaactcaccgacgacctctcccggtgggaacaaccgcaagttcgcgaaaaatgggccgaagtctttttaggcaggtaaactcaaaaccaaaaaagaaaggagcatactcatgaacaatcaaaataccacacgcatacaaatccatctgcttcaaaaccacgcaccggccaacttgaacagagacgatttgggagcaccaaaaacctgctacttgggcggcgtcctccgctcccgcatctccagccagtgcctcaaacgcagcatccgattgagcccctacttcaaagacctctgcggcggtgttcgcacacgccgcctcgcaaaactcatcactgaccaggttgatggcaacaatgcgacgaagaaaaaagtggccgccatcctgaagaaatgtggtttggaggccaaagagaacgacgaaacaaatatggtcgtttacaccacaaaagaagccatcagggaaatggcaacagccttgagaaacggactcgaagaacccgacattatcgccagattcaaagagcttttgagcacgaatactgctgtcccggacatggcactgtgtggacgcatgttggagacaggagaaatcaaggacaccaacgtcgaagccgccctgcaagccgcccacgccatttccacacacgaggctcgccccgaagtggattactatgtgacggcggatgacatccccggaactgatgccggcgctggttatgtggacgaagccatattcgtttccgcctgttattacaaatacttttccattgattgggcacaattgctccacaacctcaagggctacccgggcaacagaatccatctcgcagcccataccgtcggcgcctttatcaaggcagctgccttggtgactccatccggcaaacaaaacagctatgccgcccacaatccacccgatggtatcctcgtggaaatctcccccacacccatcaattatgccaatgcctttgccaaacccgtggacattggccaacgtgatttaatcggccaaagcattgctcaactcgcccagtacatccacgagatggatgccggtttcaaagtcgttgacccgtccaagcgcttctggttttcgccgggcatgcgcaatcgcttggagacctcacagcgcgccggtgatacagcaggcgaccgccaaaccatccgagtccaggcggtggaggttgatcaactcgaaaacctgatccatcaggtcatcaaggccattgacccgatcctcgactggcaggaagtccaaagaaccattctcaataaccatgaagcctgacacacttcttcttagactggaaggccccttgcaagcatgggggcattacgagtccaaatttgccatccgccgtgccgccgaggcccccaccaagtccggtgtcatcggccttctgctggcggccctcggcatcccgcgcacctccgctcctgatgattggttgggccggttaaactccctcctcatgggcgtgcgcgtggaccgtccaggtgtccgttggtgggactaccacacagtgggagctggccttaaaatgagaacagccgagggcaaaaacaaggatggccccctcctcacacgcagagaatttctctgcgatgcctcattcttggtagcccttaatggagagcccgcccttataaaagaactataccaagccctccaacaacccaaatggactctttacttgggacgcaagtgctgcccgccttcaaggccaatactggctcacgcccccggctgtcacaaagatctaccatcggcactacagtctgttccatgggaaaaacggtatgcagacgacaccacaccggaaaaacttgaagccctgctggagtggcgtccctccgaccaacaacccaatgcccccgatgacgccgaaatctggtatgatgccccccaatgcttggaacctcccgctcacggtccccgttttattacgagaactgccttctcagtcgctccagacggccaagtcaccgtggcgccccaacaccaacagcagcttcctttgccgcctccacgcccaagagcaaattacaacaactcagcataccaacgcgctcgtgataagcgtctgcacgccgatcatggcctgtgcgtgttttgcaaaaatcctgccaccaccgtccaacacatcacctaccgccgcgccggtggcaacgaaacaacagaagacctccgctccctctgccgcctttgtcatgatgccgtcaccatgatcgaatacggtttgggtttggagatgggccgcatcaacccggaagacccccaatggcgcgatgccatactcaagaaacgcaccgaaatcatccaattccgttcactcgaaaaccgcagacgttttttgcaaccagaggaggtttaaccatgtatttgagccacttattgattgatgtcggcaccaaccccgaccggccacgccccggccgactttggctgcgcaatatttaccatgtgcaccaacgcctgagcatggccttccccaccactcagcggaaaaccaacgacccccacttcttgaaaccctacaacccggccgattttcccctgcacaaccttggcaaccatgtttcccgcaacgaaaacaccggatttctcttccgcattgaccccgtgccggggggcaatcccctgatcatcgttcaatccagcctcaaacccaattgggactacgccttcaacaatgcggcattccttctggcagcccctccccaatgcagggacttcctgcccaatttcaacaaaggcgatcacctgcgcttccgcatcctcatcaaccttagcaaaaaagccaggacaagccatgacggcacccctctcaccaaagtcaaagaaaacatcctcgataaccaaaaccgccctaaatcccagtccaaacgtgtcgcgctgacatgggatgcaaaccaaaatccacaagatgtcatcattccttggttttcccgcaaggcatacgtcacatgccaaaaccaaacccaacccatccaggcatttcatctcaagcaatgtgaaatcctgcgcatgggttggattatcggatggaaaaaccaccatgcagccccggaaccgacctcacaaaaaatgaaattccgctctgcccttttggagggagtcttggaagtttcccatcctcaagccttcgccaaactagtagcctcaggcatcggctcggccaaagcattcggttttggtttgctctcgctctccccggtttcctccgctcaatgaaactggacgacctccatatcctccccaaattctccgatggttggagctacctctatgtcgagcattgtcgcgtcgaacgagaacaaaacgccatcgccatctatgacacagatggcatgatccccgtcccttgcgccaacctggctctcttgatgcttggcccgggcacctccatctcccatgcagccataacagttctggcggaccacggctgtttaatcatctggtgcggagaacaaggcgtccgcttctacgccaccggtatgggtgaaacccgcaatgccgcccgcctcctccatcaagccttcatgtggagccacgaaaacctccgtttcgaagtcgtccgccgtctctacgcaatgcgttttacagaaaccctgccatccaacctgtcattatctcaaatccgcggactcgaaggccgccgcgtccgcctggcctatgacaaagcggcccgtgccagcggcatcccctggaaaggccgccagtatgaccgccatcattggacccactccgaccccgtcaaccgtgccctttccaccgccaattcctgcctctacggcctctgtcactccgctattgtctcctcaggtttctcccccgccataggctttatccatactggcaaaatgctctccttcgtctatgatatcgccgacctctacaaaaccgaaatcacagtccccatagctttccaagcggccgcccaaggagacaccaacctcgaacgccgtgtccgcatctcatgccgtgacgcctttgccgaatctcacctcttggcccgtattatccctgacattcaaaaagctcttgcttggcaaccatcccaaaacgaaaactccccccttgattcagaccccgcccttccctctcacctttgggacccggatttaggggccgtgcaaggcggaatccaacaacctgccacatcccctttgcccccaactataccagccccagccccggaatctcatgatggtgctaatccttgaatcagtcccaccttcactgcgtggagaactcacccgctggctcttggaactcaaagcgggtgtcttcgtcggccgcgtctccgccctcgtccgtgagcgcctttggaataaatcctgccaccacgcccgcaatggcggctgcctcctcatctaccacaccaataacgaacaaggctttaacatcctctcctatggtaatacatccagaactatcgccaacttcgatggactcctcctcgtcaaaatcccaaaataaccccattttccaagggttttccccacgcacgtgggggtgaaccgcgtggtgcgaggtgaacaagaagatgccaagcgttttccccacgcacgtgggggtgaaccgactggctccgcggcggcgcgtggcttgaggacgttttccccacgcacgtgggggtgaaccggccgtcacctgcccctcccgatccgtccccacgttttccccacgcacgtgggggtgaaccgcggcaggtggtgaagagttcggcgaagtttgcgttttccccacgcacgtgggggtgaaccgtccacccccatcaccagccgccgccccgccgcgttttccccacgcacgtgggggtgaaccgcttccgaagttctcacggacgaagctcaggcagttttccccacgcacgtgggggtgaaccgggcatatttacggtttcacaatgaaccaactcgttttccccacgcacgtgggggtgaaccggccgctttcgaggagctgacgcggctgaagaagttttccccacgcacgtgggggtgaaccggtggcgcgccggtcaatgcagtgccatcttcagttttccccacgcacgtgggggtgaaccggggacagaatatattgtcgagccgctcaacctgttttccccacgcacgtgggggtgaaccgtccctgaccgccatgaactccacgcccgcgccgttttccccacgcacgtgggggtgaaccgccattttgatcaaattccctgcacctcaatccgttttccccacgcacgtgggggtgaacctgaggttgaggccatccgccggcgctcgaaagcgttttccccacgcacgtgggggtgaaccgatttcgctgcggctatcggcctgaacctcaaggttttccccacgcacgtgggggtgaaccgaacctggccatcgagaagaacctggccatcgagttttccccacgcacgtgggggtgaaccgtagtctggccacagcatttcttagagacaactgttttccccacgcacgtgggggtgaaccgtcgatatagccgggttttccagcttcgagggagttttccccacgcacgtgggggtgaaccggcatcgcgctttgatttcgaggaaaaatacgagttttccccacgcacgtgggggtgaaccgcctgtgcgccggtctcggtctgaagttaatacgttttccccacgcacgtgggggtgaaccgttagtcgttgtctcaccgccaccgctcgcgctgttttccccacgcacgtgggggtgaaccggcctcattcccggttggcttaaaaacggtgaagttttccccacgcacgtgggggtgaaccgtgacttggagccaccaacacacaagccagcgcgttttccccacgcacgtgggggtgaaccgacgattgcaacagcggcgttttcggacgcttcgttttccccacgcacgtgggggtgaaccgtggaggtctgaggatggcgagcacgttgggccgttttccccacgcacgtgggggtgaaccgtccaaaccgataagaacatgaagaaacacgaagttttccccacgcacgtgggggtgaaccacgcagcgcggcgacatagtggtgttcaatggcgttttccccacgcacgtgggggtgaaccgcaatgaggtccgagggctggtcatcaacaagagttttccccacgcacgtgggggtgaaccggtcattggcactcttgaccggcgtggccacacgttttccccacgcacgtgggggtgaaccgctttccaggcaccacatcaacaattccccgaagttttccccacgcacgtgggggtgaaccggaactggtggaaatctctacgttgcgcttagtgttttccccacgcacgtgggggtgaaccggcaagcctgacgccaccaccaccgccgacggggttttccccacgcacgtgggggtgaaccgcgccgccatggccggtcatctggccggccaaagttttccccacgcacgtgggggtgaaccgtgtgatccgcaggcgccggccccaacggcggtgttttccccacgcacgtgggggtgaaccgtgatgcccgcggccgcgggcaatgcagtggccgttttccccacgcacgtgggggtgaaccggtccagccggctttgcgcggggccggcgaggggttttccccacgcacgtgggggtgaaccggttgccagacaatgttgacgtagccacatacagttttccccacgcacgtgggggtgaaccgtttgttccagtgaaataccagccaccgtcactgttttccccacgcacgtgggggtgaaccggcggcgatggagctggcgcgggttgaggcggagttttccccacgcacgtgggggtgaaccggtctgcgccagaaccactagctcctaaaatatgttttccccacgcacgtgggggtgaaccagttagttccgcggcccccgtgttcgagacgcgtgttttccccacgcacgtgggggtgaaccgctatgctggccaatgtaggtatgatgagtgatgttttccccacgcacgtgggggtgaaccgtttgtttaactctttagaagaaaccgaaatgttttccccacgcacgtgggggtgaaccgaaaccgaaatggaacgcattctgcttgaacacgttttccccacgcacgtgggggtgaaccgaggtcgaagcggagcaaaaacgggccgctgaagttttccccacgcacgtgggggtgaaccgaaaattattctctgacgtcatcatattctgaagttttccccacgcacgtgggggtgaaccgtttgagtcggactgatgtggtggcggacagtggttttccccacgcacgtgggggtgaaccgttttggatcatccggcgagtaaagaagttttcgttttccccacgcacgtgggggtgaaccgccaccacctccacgatggtgcaggttggaaaagttttccccacgcacgtgggggtgaaccgagggcggcggcattgccgcacgccacgagtgcgttttccccacgcacgtgggggtgaaccggcataaccacgagcccactcttcaaacttttcgttttccccacgcacgtgggggtgaaccgatcgtccagctttttgaagaagcggacgagatgttttccccacgcacgtgggggtgaaccgaaacggctttcgccgcaacgaccacaatctaggttttccccacgcacgtgggggtgaaccgctggtggacacgacccgcaaggaacgcaacgcgttttccccacgcacgtgggggtgaaccgataccacactgtctgccgacgaagccgcccgggttttccccacgcacgtgggggtgaaccgcagattgcggtgtggttcagccgtcaccaaccgttttccccacgcacgtgggggtgaaccgtttcatggcaccgaagaccacgatgtggacaagttttccccacgcacgtgggggtgaaccgaagggctggcgtgccggtaagtaacatcatcagttttccccacgcacgtgggggtgaaccgatggaaccgtagccacggagtttgagtttgttgttttccccacgcacgtgggggtgaaccgggcaggcggacagattgccacgcctcgcgacagttttccccacgcacgtgggggtgaaccgttgttcggcccagcgctccggcagcatccggtgttttccccacgcacgtgggggtgaaccgctcggtttcacgtgcttgtcgttcacgcaaaagttttccccacgcacgtgggggtgaaccgcggtactgacgggctggtctcacgcccaagccgttttccccacgcacgtgggggtgaaccgaattgcgtataatccggaaaataacacacggagttttccccacgcacgtgggggtgaaccgaagcggaggccaaggcattggctgaagctgaagttttccccacgcacgtgggggtgaaccgagacggcttttcagccgcaacgaccacaatctgttttccccacgcacgtgggggtgaaccgatgcaatctcggctgctacgaagcgtggcacggttttccccacgcacgtgggggtgaaccagggggcaccttgaaaaggttgacatctgtggcgttttccccacgcacgtgggggtgaaccagtagtcgcctagttggattcgttgccgaatccagttttccccacgcacgtgggggtgaaccgctggtggacacgacccgcaaggaacgcaacgcgttttccccacgcacgtgggggtgaaccggcgcacataaagtgcgccaatgaacgcaagaggttttccccacgcacgtgggggtgaaccgctctttgccacagcgcttgcggccagggcagggttttccccacgcacgtgggggtgaaccgaaggaaacagcttccaaaatgccatccttgcggttttccccacgcacgtgggggtgaaccgtcttatgccagacccagacaacagccacgaaagttttccccacgcacgtgggggtgaaccgaaaagcgcggcgccgtattgttctcctgccttgttttccccacgcacgtgggggtga

SEQ ID NO: 3 - DNA, 32759 bp, unidentified
  misc_feature 1..32759: note = Ga0172376_10001016_organized_organized
  source 1..32759: mol_type = other DNA
ggggaagactagccgcgcccaaggccgtatggaatcattgtcggttcacccccacatgcgtggggaagaccaatacacaatgaaatacaagaaatttacaaacggttcacccccacatgcgtggggaagacttttccaatcggtgaagaaatttggcttttaccggttcacccccacatgcgtggggaagacggagcaaacatgctgacaccgtatccaacggacggttcacccccacatgcgtggggaagactcttaatttacagcagaaaacatagtaaacaagcggccacttggacaaatggtcatgccaacccaactcatgaagcttctggttgcccgcctttagcccgaagaagcgtcaccaaggctaaaccctcgcgttcgatcagcatccgccttgccgaaccatattgccggaactggaagccctggggcgtcgggcttgaccagatttgcgccacatagcccagaggcggcctttcggtcaccttcgcccaaagctcatccctcaccctccggctgcaatggcccaggaaaactcccggcttgatttccatcagccagcgagacagctttcccctcaagctcgtcgggattttctccgagatcatcagcagcatcgagcacctccgcgatatccggcaatatgcgcctcatcaacttggcctggtgaaaagccacacggcaggccaatcgtgtttcccgctcaagcccgggcccgtccttggcggccatcgtgaatgccatcggtaccgtcaattccgacttgtacaagtccgcgatgtcgtaaacgaagctcagcattttccccacatggatgaacccgaggccggcggaatatccggctccgacgatcgcggcatggcacaggccgtaaaggcacgcgttcgccgccgaaaggcaacgattgacagcgttggccttgaaccagtcatcctggtcgtaggtccgcccgtcccatggaactcccgttttcctgctccattcctcgtagcaaacgcggacccggtatccttccatgccccgcaactgttccattgtcttggcggaaacatcctgtcccgggaatcgcttggcatacatccggcgggcgacctgcatccgtgtctcctcccgactcaccagcatcgcctggttgatcagccgccgcgcgctgaatgtcgcgcccgtggaatgggcgtaaagcctcacgccctgctcgccaacccacgccaccatgcaggcgttgtcggccaggatcctcatcgcggcgtgggtgacggaggtgcccggacccagcttcaggagcgccaactggtcgatcggcaccggggtctcgccatcggcatcgcggaataccagcgccgaggcggtgcaatccagcttgccatgttcgaggtaaagatgtgaccagcggtcggaaaaccgcggcagatcgtgcaggcggcgcatcagtgtgaccctttgtcgcgggatggaacgcccatcgaatcgctcacgaggcatatcccgaacaaatgaggttttacaagtgaattcggcagtgatgtggccaaaaagacggggccttcccgtcggattcgcggaaatggcgccaaaacgaggtgttgtctttgcattttaaccagataaaaatgcaaagaatcaaggttcagggaaaggcgcataacgggcggttcgaacatgggttccgtcgtattggataattccgacccggcacgatggaacacgctgtcatcaccctggttggaagcgctggatagccgcggcgtcaacatccggatctcgccgctcgaggcgctcgccaattcggcggacttgtgccaacttgccttttccaaccctctcgaccttttcgcggcccacaggtttttgctgtcgctcctgtactggaaggcgggagccgctaacggcacccgggcgctgcgcgagcggttggtttcgggaaccatgccccagggcgttatcgaatcgattcgggaggaagcatccgcattcaacctttttgatcctgaaaggccattcctccaggatcccgcttgccgggacgacaaaaaggaatccccgccctcctatcttttcgccgaaatggcctcgggaacgaatgtcgcccacttccatcacggggaggacgattcatcccggctatgcctttgctgtgcgactcggggtttattgcgactggcgccctggatgcaatcgggcggcaaggggttgaaacccgccatccacggcgcgccgccgatcatggcgatcgccatgggggacacattgcgtcacaccctgggcctcaacctgattccgtttgacgggcatttgggaaatccgcaatggaccggaccattcaagccgacgcgcgagggggccgtcaccccgctcctcgaggcgttcacctggaacacgcgacgcgtccatttgctcgatccttctccgctggactgctgttccaattgcggatcggaaagcggtttcggtgtcggaccgatcgttttcaaggcgaacgaggcttgcaaggatgattcggatgaggggcaagcgtatgccgacaattggcgcgacccggcggccttttacaccgtgaaggatggcgatactgtcaacaagaccgtcaagtcgaccgacgagtcacaggcctccgtcgccgccgatctcacgcgcctgattcctcaaaagaggggcaaggcggaagtaccggcgccacggtgtctcctgacgcaggccaatccgggacacgcgaactggatccttgtcattccctgcaccaatcccgccaataacaagacattcgatcaccgcttggtggcgatacggggagagctgcccggcgccatcgtaccgggccgtaacgatgtggatgcgattcctgttcttgccggggacccgcgcacgctgcgcaagccctggtcggcgaaccccagccggggcgcgagggtattcgtgaacgccgccagccgcctgcaaaagggtgactgggcggtgttggcggggtccgccggcaagcccttgggacacgacgccgcggccttcgacatcttctcttccctttattggccaatcaggagccgggtttcctcggcgccttcgcgggaagccgcctggatgaccctcaaactgatggcggtttccaacctcggtggtaatgccgccatgggtgatattcgcccgtggaatcatttgaaaaacagtatgcggaaacagaaaagccggaacggcatcaacattgcctacccgaggcggattcccaagggcgttcaccttgaaaccgagcttggggggatcatcaggcgtttttccggcaagggactcgactgggcgggcctttgccaattcctcaacgaccatctttcctgaccacacctgaagggagcgcatcgatgtttgtccagatccacatgcttcaaagtgttccgcccggcaatctcaaccgggatgattccgggttgcccaagaaatgcgtttttggcggcgtcacccgcgggcgcatcagcagccagtgtctcaagcgcaacatcaggctttcacctctgttcaaggaagctttcgcggaacacttggccgttcgctcgaaataccttcccacgctggtcgccaacgccttgagggaaaacagggccgaagccatccctgaaaaggaaatcgaggcggtcaccgcggccttggccgcgaggttcaagaaagaggaaaaagccagggattccgaacaggacaccggggaggacgatgaggctgccgatgcccccgaggctttggcggtcactccgcaactggtatttttccgccccgcgttcgccggcaagatcgcagaactcgcgcggaacttgaggagcaccgatccgctggcctacaagcttttcaccaagatggaaagaatctcgaaaaacgatgagagaagcgccaaggccggcgtcgaaaaattcatcaggtcggtgttcgaggcgcgggaatcgttgtcggtcgatgtcgcgcttttcggtcgcatgaccaccagcgacatcgtaatggatatcgaagcctcgtgccaggtggcgcatgcgatcgcgacccatgagaccttgattgaaagcgattacttcacggcgatggacgacctcaacgacagcttgggcaagggtgccggtgcggcattcctggggtcgggcgacaccgagacattcttcaattccgccgtttattacaagtacctgaacatggacacgggcgcatttttgcgcaacctccggggtgatgacacctccgtggcctccaaggccgccgcggttctccttgaagccgcggcactctcgacgcccacgggaaaacagaactccttcgccgcccattccgtaccggaattggtgctggtcgaggtctccccaaggaagcagccgatcagttacgccaacgcgttcctggggcccgtgcaaggggaggacctgatggcggcctcggcggacgccctggctgattattgcgggacagtcgcggcggcattttcaccgccgaaaactcaaagattttaccttgccgttggcagggcccggaacttcaggatccccatggaaggtgaacggatcgacagcctggccaacttggcccttgaggttgggaaaggtgtcaaggctgccaacgcgggtgccaagtgagcgacaacaccctgttgctgcgcctggcagggcccatgcaggcgtggggcaccgattcccgattccagttgcgccggaccgaatccgttcccggcaagtcgggcgtcatcgggctggttctttgcgccatgggaatcaggcgcgaagaggcctccgcgaaggctgccgaattgtcggccttgaaaatggccgtccgtgtcgaccgggccggccacaccgactgggactaccacaccgtcggcgccggcatcggaatccgtcaagccgaggggggcgtcaagaaaacggcaagcacgggtgaaccggagacccttctgtccaggcgccagtacctttgggacgcctctttcacggtggcgcttcagggtcccggcaacctggttgcggatgccgcccaggcgctgagtgaacccgtttggcctccgttcctcgggcgaaaatgctgcgtgccaaccgaacccgtctttttggccgctggccgtttcgcaaacctcgcggaggcgctgggggcgatcgaatggacggaccatcatggatcgtcgaggaacgggacgacggaactccaggcattcattgaacacgacgaaggcggcccggtcccaccaggtgccttcgtggtttacgacacaccgcgaaccctgcgaaatcccagccatgggccaaggttggtggttcccggcacgatcaatgttcgggttgcgaccgttcaaccggccaacgcaatcggcaggcgaggagtgaactacgccgcggcccaatggcgagcggtgagggaaaggcggttggaactggaccgtttcctctgcgtcttctgcaagatgcccgcggaggatgtgcatcatgtgacctacgaaagggctggaaacgaatccctcgacgacctgaggtcgttgtgcaagatctgccatgacgcctgcacgcaactcgaatacggctccggcatgactcgggaaaggattgatccggcagaacccgcggcgcgcaacgccatcatggagcaggttggcaaacttctttcacaaaggcgcctggcaaggcgtcaggccatacgcgatttggcccgcggcgactttttcgacaatgttcccgatgcggggatatcctgaatgttcctgtcccaattgatcgtcaatacggcgggaaatccggaccaaccccgtcccggcagggaatgggtgcgatccacttacagggtgcatcagcgaatctggatggcttttcccgacgaacacaagcgccatgccgatccgtttttccttggcgcctggtcaacgcctgatggcgtcaagcctcgaaggacggaggcgggattccttttccgcgtcgaaccggatttgccgacccgggttttggtccaatcggttttccgcccggattgggaatatgccttccagaacgccaggcatttgctggcggaaacgccaaatgtgagggaattcaatccggacccagtcgccggccaaccatacaaattcaggctcgccatgctcatggtgaagaggaaaacacacaagggatccgaatcagccgggcccgtcgagcatccgatccgttgcctggttccctcgcccgtggctggcgagccttttcggcccgatccgaaattcacggcttggcgggaaaagctatcctcggaagccttgcggcatgggtttgcgctgggcgagtatccctcaaggctttcagtttcgccgatcaggcacctcatgatgaaaccggacaaggacggcaaggccatgcccttcaacgccgccatgttcgagggcgagttgacctgcaccgacccggaaaaactcaaggccgcgatggttggtggaatcggccgtggcaaggctttcggcatgggcctgctttcgctcgcgatccttcccaagaaagcctccggctagaccttcccaaggcggatcgtccggcccggaaaatatcacctcgccatcaactttgaaatcagggtggcgccgctggcgggccatccgcctgccgtccggtgacatggccccaagcccgcagccggggcccgtcgggcctgatggtgatggcgccctccacggaagtcgaccaggtgcggtccctcgggcctaggcgcgcccgccgcgaatcttcctgcgccaccgtgagccagggcgcggcccagcccaaaaacgccggcccgttggcccccgcgccgccatggtgcggcgattgcagcacggccacgccggggccgggaccggctttcatgagcgccctctggccggcgccctcgaggtcgcccgtgagcagcacatttcccgaggaatgcctcagcagcgccaccacgctcgcctcgttctggctggaggcctcgaagccgggcgccgggtgcaggaactccagcgatgccgccccgcgggccaggctgaaccccggctccacctcctccacgggaactccccgcgccgcgagtccgtcgagcatgaagcggacgccgggcacctccttgcgggcgaaccagcccgacaccagcacccggcccaccggcacccgctcgaccaggtcgagcgcgccattgaaatggtcgaggtcggcatgggaaaggatcaactcgtcaacccgttccacccccctgttccagaggaacggcgcgatgatcctcctgcccacgccggcgttggccaacgatccggcgtcgtacaccgtggtggacccgtcggcccattcgatgacggcgcatcccccgtggccgacggcaaggaaggtgcatcgccagggatcgctccgcgcggggggcgccagcgacaccgccaatcccgcggccagccaggccatcgccggcactcccgcccgggcgcgggcccgccatccgttctccgagagcgccggaatcagcaggatgtgccatggcacaagccaggcgagtgaaatgtcgcccacccagaagctggcccatggccagttggccgcccagccgaccagccccgcacagcccttgagcgccatgcccgcgcaccatccgagcgccggggaaaccaacggaacatccccacccagaatcagcaggggaaagccgggcagcagcgccagcgatcccaggagcgccacgggcggacccaccaccagcgccgaggggctgaccagatgggtgcgcgccgcgaccagcggagtgaggcagcaccagatgacggcattggttccaagggcgatggccagggcccgaaggaacgcgcgaaccagtcgccaaggagccgaccggcccatgtcccgggccctggccaaggcctcctcctgttcggtgggcggttcggaccacccgggtacggcccagtacaaaagggcggtggcgaggaacgacagcaggcaaccggtgccgaaaaggtctcccgggtcgagcaggccgaccacgagccaactgaaggcgatcaccgagggaaggtggcggggccgccgcaggatgtcggccagggccagggccccgacggttgccgcggcgcgggtggccgccggccgcgcccccgtcatgcccgcgtaaatcgccacggtcagggcgatgggccatgccaaggcgcgcgcgcccatgccggcgcaccggccggcgacggtgaggaacatcgccaagagcaccaggtgctggcccgagatcgccagcacatgcaccacgccggtgcggcggtactggtcccagctttcccgggacagccttgaagtctcgcccaacagcagcgcggcggccagcggctcaacctccggatcgatcgccccggccaaccgggccgcggccctcgcccggagcccggcgagaatgacgcggatatcccaggtgcccgagggccccatcgattccagcgcgcccgggttgtcggcgacgcggacctcggcaacgatgccgcgggccagccaaggatcgcgcccgtcggtttcccccggattcatcggcccgggaatcaggcggatctgccccgcgatgtcgacacggtcgccgggcgacgcgtccgggggcgctcccgaaccggcgacccgaacctcgcctaccgcctcggatggcgcgcccccgcgccgggcgctctcgaccgcgaccaccgtctcccagcgcgccggaaccggaatcagacgaagcggatcgggtttgggcggggaagtcgcctggatggcatccaccacgatgccgcgcaggcggattggcgcgtgctcggatccggccatcgaggcgatgtcgttcccgcccatcgaaccgaaaagcaaggcgccgcgaacggcgcccatggcgaacacgaggaacagaatccagagcggccgccacctcacatgggccagggcggccagggcgaacgccaccgccagggccatcaccacccaaggcgtcggcctggccagcctctcgaggaaaatgccggcggtcagtgaaagggcggccggcgccaagggcgccgaccgccagattccagccacgggggccgccaactccagccatcgccgaagcaaggcggccatgacgcttcagcgcgccggggccccgaaggcccaaagatacttgaagccgcgcagataaatcttgccgtcggcgatcgcaggcgaggcggccagcacatcgggcaccgtgttgtcggcgaccttctcgaacttcttgcccgccttcaccaccgtcacggtccccttgtcgcggttggtgaccagcacatggccgtcaaccagcaccggcgaggcgcggaaccggccggcggtggtgcgctcctggtagtactcctgacccgacgcggcatcgatgcagatcagcgtgccgttctcgcggcacaggtaaaccagtccgtccgccaccaggggcgacggcacatccggggtgttgctgggcttgcgccattgctcgtggcccgacccgggccccacgattccggtggccccgggcttgatgcccacgacctgcccgttcttcgccgtgggcacgacgatgaggccgggtgccgcgacgggcgaggcgacaaatcgcagatacttgttgtacttgtccttggggttcaggtcgatcacgcgccagatctcatggccgtccttgaggtcgtgggccacggcgtggtcgttgccatgggtgacgaggtaggccttgccgccgtcctgccaaaggacgggcgaggcgtaggaatgcgggttctcgtcggtgccctcgctgggccgggtcactttccagacttcctttccggtggcggcatcgagggcgatcaccaactgggcgcccgtgtgcagcagctggacgaagagctttccgtcgtgcagaaccggcgtcgagtgcatgccgaactggatgttgaacttgccgtatttctcctggaggttcaccgcccaaaccttggtgccgtcaaccgcgtggcaggccaggtctccggaggcgacgaaggcccacacatgcttgccgtcggtcaccgccgaggccgaggcccagttgccctcgtcggtgcgggccgggacattgccggatcccagcttggcttgccatttttccttgcccttggtgtcgtacgccaataacagcaggtccttctccacgacgcaggtgaggaagatcgagtcgccccacacgacgggggtggagcaacccatgcccggaagcggcgccttccacagcaggttctgggtttcgccccatgccgtgggcaggcccttctcgggagagtggccatcgcccaggggaccgcgccaggtgggccaggggcccgccaaggcgcccgccgccacgcatcccgccattgccaccgccatcaatccgcgcatgccttcttcctcaaccggtttcacgagtctccgtggccaagagtgtaacgggttcgaatccgcccggccagcggttcccgccagccaaggttcatgaaatcttccaacacgggaacccgaacatcgggttgaatggaactaacgatccgacaccgggaggagccgaccatgagacccctgatctgttgtcttgccgtggcgctggcgacggcggccgttcacgcccagatggagccgaactttcccggccccaagcccgacggttccgtccagcttcccaaccagtggcgcctcatgcccgcgggcaggcacaccacgctgggcgattatcccgtgcaggcgctggtgcatcccaagggccccttcgtcgccgccctgcactgcggctacggcaagcacgaggtgatcgtgctcaaggtggaaggcgacaccaccttactggtcagccgcgtcgggttcccccaggccttccacggcatggcgttcgacgccgccggtgaaaccctgttcgtgagcggcggcgagttcgaggtcatccgcgcctacccgttcaaggacggccaactcggcacgccgcgcgaactcaccgtggtggacaaggcccagaccttcattccctcgggcatcgccgtcatgcccggcggcaagtcgctggtcgccgccggcgtgctcggccactccgtggcccaggtcgacctcgccacgggcaagggatccaagcccattcccctgccggaaggatcgtatccctactgctgcctgcccctggatgacgacgaggttctcgtctcactatggggcaagacaatggtggcgctggtcgacctcgacgacgccaaggtgaaggcggtctgggacctcggcctggcccaccccgccgaaatgtccttgtcgaaggatcgaaagactctctatgtggcgtgcgccaactccaccatggtggcggtggtcgacgcggaatcgggcaaggtggccaagaccctcaactgcgcgctccatccaggggcgccggtcggcaacacccccggaagcctgacgctttccgccgacggcaagctgctcgccgtggccaacgccgacgccagcaacctcgccctgttcggcctcgagtcaccgcgcgatcccaagcctctgggcttcattcccaccggttggtatcccacctccgtgcggttccaccatgacggcaggctgctggtcaccaacggcaagggagccagctcgaaggccaatcccaagggccccaaccccctggccaagaggcaacccaccacggaatacatcggcgggctgtttcagggcacgctttcggtgatcgacgcccccactccggaacgcctcgccgaatggacccccaaggcttactcgtgcagtcccctgcgcgccgatgccggtgccatccaggcccgccccgagggcaaccccgttcccgcgaaaatcggcgacaagtcacccatccgccattgcgtttacatcatcaaggagaaccgcacctacgaccaggtgttcggcgacatggtgccgggcaacggcgacaagtcgctctgcctgttccctgaaaagatcacccccaaccaccatgccctggcacgcgaatatgtccagctcgacaacttctatgtcgacggcgaggtttccgcggacgggcacgaatggtcgatgggcgcctacgccaccgacttcgtcgagaaggtctggccgctgggctaccggggcagcccgctcaagaagttcgccttctacccggccgaaggcaccaaggatcccgtcgctcgccccgccggcggctacatctgggaccgcgccgccgaggcgggcgtgacctaccgcagctacggcgaatggatcgccgacacggcgcccggcaagccggccaaggcgcgcgtcaaggcgctcgaggggcatttcgaccctttcttcccctcctacaacctgtcggtcaccgatgtcaggcgcgccgaacggttccttgaggaactcaccggatttgagaaaaccggcaagttcccgcaactcaccatcctcaggctgcccaacgaccacacggccggcacctcgcccggcaagcccacccccaccgccatggtggccgacaacgacctcgcccttggcatggtgatcgagggcctctccaagagccggttctggaaggaaatggcggtgttcgtgcttgaggacgacgcccagaacgggcccgaccatgtggacgcccaccgctcggtggcgttgtgcgtcagcccgttcaccaagggccgcggcctcgactcgaccctctattccaccagttcgatgttgcgcacgatggaactcatcctcggcctcaagccgatgagccagttcgatgccgccgcccggccgatgtacaactcgtttctccccaagggcgacgccgcgccctacaaggcccgccccgcgggcaccgacctgaaggaactcaacacggcgcaggcctggggcgcgaagcagtcgctggcgttcgacctctcgaaggaggacgcggccgacgacctcgccttcaacgaggtgatctggcgctcggtgaagggcgccaacagcccgatgccggcccccgttcgcgcggcgtttttcctggccaagtgaacaaagcaaagaggctgtttcgctccggccccgtgatcgttcaggcaaggacaacaaacaaggcgccgggttcacgcccggtgccagcctgtttcatggggctggctgccgattggcgacgctgacacgacacaatttacacatggaagatcgtataataattcattgaaagtgctgcgaagctccggagtgctgcttcacggagtgggaaccccatggcttgcgccatgggctcaccaaaagtctcccccggttcaccaaacttcccggtgttgtcagttattgtgccgccttggggtgagcgccgtgcgtgagcgcggcgatacgggccagctttcgggaaccccatggcttgcgccatgggctcaccaaaagtctccctcggttcaccaaacttcccggagttgtcagttattgtgccgccttggggtgagcgccgcgcgtgagcgcggcgatacgggccagctttcgggcccctttggattttgggagtgcgcactcgcattggccttgtcggcatcattcttccaaatcaccgacttggccaaaaaaaccaagttggcggcgctgacacgacacaatttacacatggaagatcgtataataattcattgaaagtgctgcgaagcaccggagtgctgcttcacggagtgggaaccccatggcttgcgccatgggctcaccaaaagtctcccccggttcaccaaacttcccggagttgtcagttattgtgccgccttggggtgagcgccgtgcgtgagcgcggcgatacgggccagctttcgggcccctttggattttgggaatgcgcactcgcattggccttgtcggcttcattcttccaaatcaccgaattgaccatgaaaaccaagtaggcggcgctgacacaacacaatttacacatggaagatcgtataaaacttcattgaaagtgttgcgaagcaccggagtgctgcttcacggagtgggaaccccgtggcttgtgccatgggctcaccaaaagtctcccccggttcaccaaacttcccggtgttgtcagttattgtgccgccttggggtgagcgccgtgcgtgagcgcggcgatacgggccagctttcgggcccctttggattttgggaatgcgcatttgcattggccttgtcggcatcattcttccaaatcaccgacttgaccatgaaaaccaaggttgggcgatctgccacccagtcattcacttcaaatccgcaggcaccggttcggcggctggtggcgggggcaccttcaggaactgcgccaggatcgccgcgctggccaagcacgaaaccttgccctcgcgcttgcccggcttgatgcccggcccgtagaccaggagcggaacatgggtgtcatagtcgtagggcgtgccgtgcgaggtgcccgtcggtatcgccgaccccccgaacaggcagccataggcgggcagcaccagcaagtcgcccgagcttttcgggtggaagctcaggcgcgcctgacgaagcaaggagtcgtcggtaggcgcgtccgattcaagctgcccccgcgtgtagcagcagccgatgccatcctgcttgcgcaaccaagttgcagcggcctcgcgaaccttggcgatgtcgccgccggcgaacgccttcgcgttcaggtacatgttttcatcgaccgggccgacaagcggctgaaccttctcccccaagtcgaattcctcacgaagatacgcaaccaacccttgtcccagcttgacggcatcaatgcgcctggcggccttggcgcccttggaagtcgccacctccggcagcggacaaacaccatggtcggctgacaagaccacgccatagttgtccgggcccaccagggcatcgagttccttgaagaaacgcgaaagcagagcgtcggtgcgcagggtgatgtcgagcacctcgtggctgtcggggccccacatgtgtcccaccacatcgttggccgaaaaactcacgcacaaaaggtcgggcacatcccgctggcccagcttttcccgccggacggcctccagcgcgaaatcggccaaaagttcattggaaaacggggagttgtatagcgcgctgtaataggtcttgtccggcttgcctgaatcggagggaaagggatgcggaaaggcgcgtccctgcttcttggcgattcccggcccttcgcccacaacatcatccggcccggcaagcttgtcgtagtcgaggtcgtcgcgaaaccggttccaggtggtgccgaaccacctgtcggccggcttggcggcgttgaaggctcccacccagtcggccacgcggtcgcggtagtaggtggaggtgacgaaatcacctgaatcgctgtcccaccagtagacgatgtttcccttgagtccgccgggaagcacggcgccccggtccttgagggaaaacgaggcgatcttggagccgctttgggcattggcgcgaatcgcgtcacccactgttggcgcccgaagcctttcgggagaaccggcccccttccggactgtggcgacggccgctgtgcccggctttggcacgacgggcttggcgggtggcacgcgctggtgcctctcggtggtggcgcagtacacgcccgcgccggtcaacgggtcgcgccaatcattgccgatgatcccatgcaccgatggactggacccggcgacaaggctggcgtgcccggcgccggtgacggtgacggcatagggatagtgggcattggcataccaggcgccctgctccagaaagcggttgaggccatcgggcccatagtgtgccttccatcgagccggataatcgccgcggaactggtcgaacaccaaaagcaccgccagcttgaccggcttgtcggtgccaccgacaggcgcctgggcccatgcggcggctggtatcatgaacggtaaaagaacaaggaatcgtgtcagcatgcatcgctccccaataggcctctctccctacaggataaccctgcgaaaggcggatgggatgaagggatgaagaaggaccgaggtggtgggggaagattcggaaaggtaaactcgaatccgcattccgcccgggggcccattccgtgccaaaacatgcgctagcgttcctgatcctcgcgtcaatcgcggactcaggcatggccgctgacttcaactcccaagccaagccggtgttcctggcccggtgcgcgcggtgccatggcgccggcacggccaaggcaggtctcaggctcgactcggccggcggggcgctccgcggcggtaccgacggcccggtcatcattcctggccaggccggcaaaagcccgctcatggcacgcgtcacaagcgacaagcccggggagatgatgccgcccaagggaccgcggctttccccggctgaaatatccgcgctgcgcgagtggatcgattcaggggccaagggaggcaacccgggggaagaccgcgatccccggcttgaccactgggcctggcaacccgtggccggcgtggcgactcccgctggcaacccggtcgaccatttcatcaacgcccgcctcaaggccgcaggcctcccgcccgccccgcgcgccgacaggcgctccctgatccgccgactgtcgttcgacctgctcggcctgcctccctcacccgaggaagtgacggcattcgtgaacgaccctgatcgcctggccttcgccaagttggtcgaccgttacctcgactcgccccggtacggggaacggcaggcgcgccactggctcgacatcgcccattacgccgacacccacggcttcgaacgcgaccagcgccgggagaacgcgtggcgctaccgcgactgggtgatccgcgccttcaacgccgacatgccctacgaccgcttcctggccgaccagatcgccggcgaccagttgcgaccgggcgaccccgaggcggtggcggcgacggggttccttgccgcgggaccctgggactttgtcggacaggcggaaacccccagccccgtgctcaagcgccaggcgcgcgccgacgacctcgacgacatggtcacgcaggtgatggcggcggcatgcggggtgacggtgaactgcgcgcgctgccacgatcacaagctcgagccgatatcccagaaggagtattacggcctgtgggccgtcttcagcggcgcgcgccggggggacaggcccctcgatccaccgggcgaggcgcggcgcgacggcgagaggaaggccctgctggccaaggccgccgaactgcgggcaacgctggcccgcctcgacggcaagggcctcagcctggccgatgccgttggcgggggcgacgggctgggctcgggaacccggggcgacgggatcaatccggcgaccggcaggaagtcagccaccaaggccggtctcatcagcggagccgtggcgaaccaataccgggcgctgggcggcctggtcgacggcgtcgtcgtccccggggggggcgatgttcccgtcagttccaccggcatcgtggccaagtcgatccccaagacatccgccaccgcctgggatgccatccgcaacggcccggtcaaccagcaaaggtcgacatccatcgccggcatcgactactcgggccccgggcatgacatcctcggactgcacgccaacgccgcgatcaccttcgacctggccaaggcccgcgccgccctcaaggccgacaccctcaggttcaccgccatggcgggatatggcggccggccgggcgacaattccaccaaggccgaccttcgcgtgctggccgatggcgtggaggtcgagcggcacctgggcctcaacccgggaagcggcccgttgcccatcgacatccccctcggtcccgacaagcggtttctcacgctcgtggccaccgagggagccgacggcggcatcggccacgaccaggtcttcctgggtgacgcccgcctgctcccctccaaaacggcccgacccccggcccctgaaacggcgaacaggcgcgagctttccgaggccctggcggccatcgagcggcaaatcgccgccatcgcgcccggcccgatggtgtacgccgtcaacggcggaacgcccgaggccgtgagggtgctcaagcgtggcaaccccgaggctcccggtgaggaaacgggccccggggcgctttcatgcctcaagggcgcccgcccgctggaggccacgagcgacatgggcgacggggccaggcgcgcggcgctggccaggtggatcgcgcagccggaaaacccgctcaccacccgggtcattgtcaaccgcctctggcaccatcatttcggagccggactagtcggaactcccagcgacttcggggccggcggtggcaagccgagccatccggaactgctcgactggctggcgggcgagctggttcggcggcgctggtcactcaaggcgatgcaccgcgtcatttgcgacagcgaggcctaccagcgatcatccatggcgcaagatcccaaggccgccttggccgacgcgggcaaccggctgctctggaggcaaaacccgcgccggctggatgccgaaagcctccgcgacgccgtcctggcgacaagcgggcagctcgaccccacccccttcggcccgggataccgcgacttcgtctaccaggaggcctacgcccccatctaccggcacatcgatgacgacagcccgtcgctgcgcaggcgcagcatctaccgctttgtcgtgcgcaccactccccagccgttcctcaccacgctcgattgccccaacccggccaacctctccccctcgcgcaccgaaaccaccacggcgctgcagtcgctggccctgctcaacaatccgttcatgctgaggcaggcccggcacttcgcccagaggctgtcccgggatgcgggtgattctaccgccgggcgggtcgggcgcgccttcgccctggcgttcgcccgcgcaccgtcacgggccgagctcgacgcctcgatggcgctcgtccgggcccgcgaccttcccgagctttgccggatgcttctcaacgccagcgagttctgccatgtcgattgagaaccttggcaggaggcgctggctgcggcagggcggatgctcgctgggcgccatcgcgctgggcacccttctggaacgcgacggactcgccgcggcgccctccgcgttcgccccaagggcaccccaccacgccccgcgcgcccgcgccgtgatcgagatcttctgccccggcggcatgagccatgtcgacaccttcgaccacaagcctgaactggaaaaatcccacggcaaggcgttcgacgccgagctgggcaagcagaccttcgccggcacgggcggaacctacgccaggagcttctggcccttccgcaggcacggccgatgcggcaaggcgatcagcgacctgttccccaacctggcccggcatgtcgacgacatcgccttcatccattccatgaggaacaagtcggcgctccatggccccgcgatgttcatgatgaactcgggcttcatccttcccggcttcccgtcgatgggctcctgggtcacctacggcctgggatgcgagaccgacaacctgccggccttcgtggtcctgcccgacatccgcggcctgccccccggcggcgtggtgaactggggcgccgggttcctgccggcggtgcaccagggcacggtggtggaggcctcgggaacccgcgagccgatcgcggacttgtttcctccggaaaggcaaggcgacgaggcgccgggccgcgcgttcctgaggtcgctcaacgccaaccacgcggaggctcgcgcctccgacacccggctggccgcgcggatggccagctacgagcttgccgcccgcctgcagatgagcgcgccggaggccgtggcgctcgaccgggaatccaccgccaccagggccatgtacgcgctcgacgacgaggacatcggcccgttcggcaggcaatgcctgctggcccgccggctggtggagcggggcgtccgcttcgtgcaggtgttctgcggcgccgagaacacccaggcccgcaaggtccgccccaactgggactcgcatgaggacctggtccgcgaccacggctactggggcagggtcctcgacaggggaacctcggcgctgctggccgacctcaagtcgcgcggactcctcgacgagacgctggtgctctgcgccaccgaattcggccgccagccgttcctgcaggggggcaaccggggccgcgaccacaaccccggcgcctacaccgcctggatggccggcggcggcaccaagggcggaacctcatacggttccagcgacgaactcggatggcaggccgccggaaacccgacctactcctacgacctccacgccaccgcgcttcaccagctcggcatcgaccacgagaagctgtcgttctaccacaacggcatcatgcgccggctcaccgatgtgcacgggaaggtcatttccgaaatcgtcgcctgactccggcgcgcctcaggcccgcaacgccgcgagcgcgccgcaatgaacccgcgtcggggcgatcgtgcgggccggcgtgaaaccggtggccgaaagcatcccccggtactccccggcgctgtaggcccgcccctcggtgagcgagaacaacgcggccgaataaagcgccacctccaacgggccgtccagggcgtcgttcaggtagacatcgtggatgaacaacctgcctccggggcggaccgccttgtggcagcggcgcaggatgtcgaggcaggtctcctcgttccagtcgtgaagcacattcgagagcagcaccgcgtcgcacgatgggtactccccgaagaggtctcccgccaccatcgccacccggtcggcggggccatgttccgcgaggatcccggaagccaccttgagcacctcgggccggtcgaggttggtaacgcgcaggcccgggttgcgctgcgccagggccattccatacaccccggttccgccgcccaggtcgaccaggtggccgatgccgtcaaggggcagcacctcggccagcacgggagcgcaaatgaaggcccggcccgccagggccatggtcaggcggcgggcgctgtctcccgattccatggccgattccatcccctcgcggaagatgaacgccttgccctcgtcgggccggttgctcaccggcttgtcggcccgcagcaggtcgatcatggccttcacgccgggcagtgcggaggtgaggccgacatagccgcggatctcatggctgccgcccggcaacaggaaccgtcccatgggagtgatggcctggggcaggcccggcgccccctcgatgagcctgagggcccgcatggcggtggaaagcacggcccacgggcggggcgccaggcccagcttctcccgcagggcatcctccggaaccggcccgccggccaacgcgtcgaagaggcccaattccagcccgctggccaacagttcggtcatgtagttgccgcgaaccatgtcgaacagcggcgtgggatcgagttcgggggtggtgaaggggggcatgatggtctccggggccggctgggcctcgaccggcgccgcctgctaacctgagactaaggcaggcgggggcgtcggagaaggtgcggatgtccgcgaaggatcgaacggcggtggttctgctcagcgggggactcgattcctccaccgtgctggccctggccatctcgcagggctaccgctgccacgccctcaccatcgcctacggccagcgtcaccaggtggaactggccgccgcggcacgcgtggccggtgccgcgggcgtggccgggcacaagacccttgccctcgatctgcggatgttcggcggctcggcgctcaccgccgacattcccgtcccccacgggcgctccatggattcgatggcttcgcacattccctgcacctatgtgcccgcccgcaacacgatcttcctgtcactggccctggcccacgcggaaaccctgggggcgttcgacatcttcgcgggcatgaacgcgctcgactattcgggctatcccgactgccgacccgagtacctggccgcgttcgaggcgatggccaacctcgcaacgcgcgacgccgtggagggcaagggccgctacaggatccacgcgccgcttttgcggttgggcaaggccgacatcatcaggcttggcgcggccatgggggtggattactcactcacccacagctgctacgacccgaccccggccggaaaggcctgcggcacctgcgacagttgcctgctgcggctcaagggcttccaggaggcgggaatgtccgacccggcgctggccgccacatgagaatcgccgaactgttcctctcgctgcagggggagggcctcctcgccggggtaccctcggtgttcgtccgcacctcgggctgcaaccttcgctgcggcttctgcgacactccctacacctcgtggcaaccggaggggctggaacgcccggccgccgatgtcgccgcggaggtgctccgccaccccacccgtcatgtggtggtgacgggcggcgagccgatgctggcgcccgacctgcccgaactcctcgaccccatcgccggcgccggcaggcatatcaccatcgagacagcggcgaccgtgtaccgtccggtccgctgcgacctggcgagcctgagccccaagctggccaacagcgcgccccgcgaccgcgaaggcggccgcttcctggccatgcatgagggcacgcgccggcgaccggatgtcatccgcaggttcatggaccaccacgaataccagctcaagttcgtggttcaagatccttccgacctcgtggagatcgacaacctcctcgatgacctgccccccgtgccgcccgagcgggtcctgctcatgccgctgggagtcaccaacacggaactccgggaacgctccgcctggctcgccggcctatgcctgggccggggctggcggctttgcccccggttgcatattgaactctacgggcacactcgaggcacctgaggcctcgaaacccatggggatggaacgatgatcacccttctgatggcctgcctggcgatgctccccccgccggcaaaacaggaacccctggtggtgccccaccggggcctcctcgggcacgctcctgaaaacacgcttgccaatttccgggcctgcctcgagctggggatggggttcgagttcgatgtccagcgctccaaggatggcgcccttgtctgcatccatgacgacacgctcgaccgaaccaccaacggcaagggccgcgtggcggacatgccgctggcttccatccgcgaactcgacgcgggagcctggttcaatacccggttcaagggggagaaggtgccgacggtggaggaggtgctgtcgctggtcggcgcgcatcgccacaagggggtgctcgtggccgtcgacctgaaggcggcccaagtggaggccgaggtggcggccatggcggtgaaagccggggcgctgaaaaagctcgtgttcataggccgggccatcagcgaacctgccgtgcgcgccaacatcatgaagcaatgccccaaggccgcatgcgcggcgctagccaacacgccggccgacttcgacgcggcgctcaccgccgccgacgcctcttgggtttacttccgtttcatcccgacccccgggctcatggagcgcgtccacaaggcgggaaggaagggattcatcgcgggtgtcacggtcgccggcgttgagccggccaactggaaggcggccgcggccgcgggcatcgacggcctgctgaccgatcatccgcttgaattccatgagaccttgcggccttctcggccaaaaccctgaccccgggccgaatcccggccgtttcaaggtgccacatgcggatcgtgtccctgcttccaagcgcgaccgaggtcgtgtgcgaactcggcctgggcgaaagccttgtgggtgtcacgcatgaatgcgactatccggacttcgtttcggcgctgccgaaagtcacgcgggcgctgatccctcacgacgccaccagccttcagatcgactcgctggtgcgggagagcctgcaagcccgcaaggccctttacgcgctggacatgcccgaactggaacggttgcgccccgacctgatcatcacccaggcgctgtgcgatgtgtgcgccgtggccgaggccgatgtggccgccgccgctgcctcgctacccggccggccgaaggtgttgaacctcgagccgatgtccatcggtgatgttctggagacgctggccacggtggcgaacgcggccggcgcgccggctcatgccaaagcggccatccgacgactgcggacgcggatcgacgcggtggccgaacgctcaaggaaggtcgccggccgcccgcgcgtggtcgtgctggaatggctcgacccgccgttcagttgcggacactggacacccgaactggtggatttggccggggccgacgaggtgatctccaggcccgggcaaccgtcgcggacgctgcggtgggatgaggtggtcgccgcccggccggatgtgctgttcatcgcctgttgcgggttttcggtggaacgcacgctggtcgaccttcccggcctcgccgcccggcccggctggtcggacctgcccgccgcgcgcgcggatcaggtctttgtcacggatggcaacgcctacttcagccggcccgggccgcgcctcgtggacagcctggaaatcctggcccacgccctgcaccccgctattcaccccctgcccgaaggcccgcggcccgccatgcgaataggcacccgccaacttgccgaaggccgggcggccctgccatgagccccaaggcggagacatgggcgggcatctatcaccggcccgggatggcccgcgccgtgtcggaatggcttggccattccaatgatctgatgtcctcaagcgggatattcctttgagccatcccgaacatccgctggtcgagggggtcgattaccatgtcgaggatggccggtgggttttcagcgcccactaccacctcaagcgcggatactgctgcacaagcggttgcaggcattgtccgtatggcaattcgcccaccgaccgggacagggccgcgaacgatggcgcccggccgtcgggagccccgggccggccccgttgaagcccatcacgattcaccgaacccgaaggcggctcgaaatcatccgacggtagtcaatccggcctcgcgcagcgccttttccatggcgggtttcttcgccgggtcgaggggagcgatgggcgcccggcacggcccgatcgattctcccagcagggccagggccgccttcactccgcctggcggcgtgcaaaggttgagcgccaggcgcagcgggtgcaggcgggcctgcgccgcccgggaaccttcccgatcgcccgccatgaacttttcgtagatctccacgagcaacttcggggcgatgttacccgtcgccggaatcgcgcccacggcgccgaggtcgagcgcgctgtagatgagcgtgtcgcgtccctgcagcaccgagaatgagggaggcacacggcggatcaggtcggccaccagcgtgaggtcgccgctggaatccttgatgccgacaatgttgggaacagccgccagtttctcggccgtctcggcctcgatgcgcaggccgccgcaggtgaccggattggaatacagcagcagcggcagcccggtggccccggccacccgcctgtaatggtcttccagttccttctgggtgggcatgatgaaatagggggtgatgaccgagaccccctggacaccctcgcgctccgccatgcgcgtgagcctttcggcctcgcgcgtggtctcggcgcccgtgcccgccagcaccggcaccctgccggcgacatgctccaccgcggtcgcgatcaccgcctgtttttccgcctcgtcgagcgcgtagaactcgctgttggtgccaagcacgaacaccgagtgcacgccgccggtgatgagccggtcgatgaaccagcgaagcctgggtaggtcgagttcccccgactcggaaaacggggtggcgaccggcggaatgatgccccgaaacgacatggcctaccctccaagtcaggaaatggcgacatccagcccgaacggcttttcaccggtgtaggcggcgtcgaggatttgcacgggatggccgacccacacgcggggggagcggtcgcgcatgtgccggtcgatctgcaacaggcagccgacattgccggtgagggccgcggaggcgcccgtggcggcgatgttccccgacttccgctgccccagggccgccgacatttccggctgggtgatgttgtagctgcccgcggcgccgcagcagtgctcgttctcggcgaggggaaccagcgaaagcccgtccaccatcgccagcacctgccggggcgccgagcggatcttctgggcgtgggccaggccgcaggcgtcgtggtaggtggccttgagcgagaccttgtggcggggcttcatggggcccagttcggcgaggaactcgtggatgtcgcgcaccttgccgccgaacacggcgccgggcccggcggcgtcgtgtccttccaggatgtggccgtagtccttgaggacggggctgcaacccccggcgttgctgatgatggcgtcgaggccggccaatccgccgggaaccgccgcgaacgagtcgcagttgccggccgccagcctggcggcgtcgtcgtcgaggccggcgtgcttgtgcagggcgccgcagcaggcctgcgatcgcgggatgtgcacatcgcagccgttgtgctggagcacccgcgccgtggccaggttgatctgcggatggaacgcgtcggcggcgcagcccagcagcaggcccaccgtggcgcgcttgggcccgatcgccggcaggaactcgggaaggttgtgatagggcgccaggtcgggcacgatctcctgcatccgccgcaggaagccgggcatcaggtatcccgaaagcctcacggcccagccgagcccgagccgttgcatcagcttgacgggaaacagcgcccagcccatgcggccgcggtgcggcgtgagcccgaacagcatggcgcgctgcagccagcccagcatgggcgcggggccccgcgtgcgcgcctggtcgttcttgaacgagtggatcacatgccggtactgcacgcccgaggggcaggcggtctcgcaggccatgcactcgaggcagccgttcatgtgcttctcggcctcgggcgagaacggcgcgcggccgtcggcgatggcgcgccagaggtagatgcggccgcgggggctgtcgttctcgttgcccgtctccacataggtggggcacgatcccaggcaaagcccgcaatggacgcagtccatgaacagctcatagtcgatgttcttgccaaggtcgctggcgatgggcaggctttggcgggtggctggggcgctcatgatggctttccgtcggggaggcgcgtgttgtggcgcatgcggttgggattgaagatgcggtcgggatccatttccctggcgatggccgcggagatggcctcctcgggccgggcgggtccccacatggagtcggcctgccgccatgggcgcggggcgcgcgcgaggcgggcccatcctcccggcccggcggcgtcgcgccagcccggcagcaggccccgggggtctttcccctcgggcaccaccgccctgaccacgcccgagccgggcaacgcgtgcaccaccgcgccgggcatgtcggccagcacgcgggccgccaggcccgccgtccgcgagggcaggcaacccagcgccgcgaccgctcccgactgggggtcggtccgctcggccaggcgcgtcgccatggcgtccaccgaattcggctggcactcgatgatctgcacatgggccaggtcgcgaaactcctccttggccctggccacctgccaggccaccgcgggagcctcgccctcgaatccgatcatcgccgtccacgcggccggcccgccgggaagggcgagaaggtcgaacgccacgggccgcgcggcgcacccgtgggcgcggtcggcggcggcctcgtgcatccccgccttcagcggaatcgccagccaggcgcgcaccggcggcaacgggcgcaccttgagcgtcacctgcgccatcggcccgagcgtgccgtgcgccccgacatgcagcttggccatgtcgtagcccgcgacattcttcacgacgcgtccgcccgacctcgtcagcttgccgttgtcgtcaaccacggtgaggccgatgacgaagtcgcggggcgttccgtgggccagccgcctcgatcccgagacattgcacgccagcaccccgcccagggtggccttgtcgggccacgggatgtccaggggcagggtttgccgttccctggcgaggatctcctggagcctcgacacgcggatcccggcctgcgcggtgatcgtcatgtcgcgggcgggatagtcgatcacgcggtcgagggagctcatgtggaccacggtgccgggaaccgtcgcgggtatgccggcgcccagggaaagcccgccgcccacccagtacaggggctggcccgaggcgcgacaggcccggacaacctgggccagggcttcgggcgtctcgggggcgacggcgtccaggtcgaggccgtcgatgtcgaatgtggggtccatggcgtgctgctggtcctcggggggagcgtcagagggcggcgcgtcggccgggcttcacctgcttgtgccgccccgcttccgcgcaggcgccggcggtgggcagcatcttgtcggggctgcacaggccgtcggggttgaatgcctgcctcacccgcctcatcgcggcgaggtcgtcggggctgaacagcttggccatgaagtcgatcttctccacgccgatcccgtgctcgccggtgacgctgccgcccagcctgaggcactcgtcgaggatctcgtggctggcctccatcacgcgcctgagttggtccgggtcgcgctcgtcgaacagcaggatggggtggaggttgccgtcgccggcatggaagacattggcgattctgagcttgtgcctgtcgccaatcgccgtgatcgacctcagcatctcggggagggcggtgcgcggcacaacgccgtcctgggtgcagtagcttggcgccagccggccgacggccccgaaggcctgcttgcggcacttccacagcagcagcctctcggcttccgtgttggcgcgcctcacctcgcgggcacggttggccttggccagcttctcgatccgcgcggcctcctcgcccaggccggcatccagcccgtccacctccatgatgaggaccgccccggcatccagcgggaaaccgaagctgaacgcctcctccaccgccttgaggatcaacgagtcgagcatctcgagcgccgccggcacgatgcccgccccgatgatggccgagatcgccgccgtggcgtcgtcgaccgactcgaacacgcccaggagcgtgcgcaccgactcgggattgcgcaccagcctcacccatgccttggtgaccacgccgaaggttccttccgaaccaacaaccacgccggtgaggtcgaggccggggccgtcctccaccgggccgccaagggtttgcaccgtgccgtcgggcatcacgatctcgacgcccatcacatggttcaccgtgacgccatacttcagggtgtgcgggccgcccgaattggtggcgacattgccgccgatggtgcaggcgccctggctggagggatcgggcgcgtagtgccaaccctcggacttgatccggttggtgagccacatgttgatcacgccggcctcgacaagcgcgaaccggttgcgcaggtcgacttccaggattcgcttcatcctggccagcgagatgatcaccccgccgcccacgggcaggcaccctcccgcgaggctcgtccccgaaccccttggcaggaaggggacccgcaactcccggcatagcttcacgatctcgaccacctgttcggtggccgtggggaacacgacgacatcgggccggttcttttcgagcgtgtagccgtcgcactcgtatacgagcaactcggcctgggagttgatcaggccgtccgggcccacgatgccggtgagccggcgcaccaggttctcgacgacggatggctcaacggcggcgacggtcatgggggggcgttccgggaactaatatcggcctttcccagcatacagggccggaaggatttcctaatagcggaaaccgttgcgccgcacatgggtccacggctcaccctcgggttccgatcggcccgcgagcaccgtgccggtcaccaggtcgtggtcacccacggggtatcggccggaaacctcgcagaggagatgggcgtgggcatcctcgagaaggggcggaaggtcgtcggaaaacctggtggccagaggggcgaatccctcgggcccgggcggcgttcccttgccgaaatgcctcaggagttccttctgtccgtcgcgcaccacatgaaccacgaaacgcgccccaggaaccagaagcggccccaggccccggtcgcggccgatcgccacgctcagggaagggggctcaaacccgcactgctgcacccagcttgccagcatcgcctgggccgaagcccccgtaccggcggtcacgatgtacaacccgcttgagacgcgccccagggccccacccagcgtgtcgcggtcttggctcagcattgtccgctcctccgcgacgatcaatccttgaccagcttttccaaaagctggggcaattccgattcggccgcctcgctgtcgaggtatttcttggcgatcagccccgccttgttgaacacgaaggtcgccggataaccctcgatctcaaacttcttctgccaaacctcaaggggctccctgagcatgtaggaaaccccgggcaacttggcgcgcctgacgatggcgtccaccttctcgcgaaccttggggtgtccctcctcggcatcgagggggatttcaacatggacggcgatcaccgtcacttttccaggaaaccggccgcccatctcgaccagatggggcaaggccttgatgcaggggattcaggtcctctgccagatgtccaccaccacgacctggcccttgaggtcgcgcacggccttggccaactcggggaaccgaacctccttgagtaccggggcgggatcctgggccatcactcccgccagcagcgcgcagcagattgaaatcatcggttcgtctccaagctatgtgtcaaggccggtaacacgactcacttcgcgaacacgcgttcgcccggcttggccgcctccgcgtcgccatcgtgcaccagcatggcgtaggtcagcttgagggtctcgcccttggccagcttcacgaggtcggcgcggcccttcatgtccgggaagccggacctgttcctgccaaacgggttggcggccatcagcccgtagccgcgggcatgccagcaggtgggatatttgttgtccgggtgggccacgatcgccacgccggcgaccttgccgccggtggggccggagtagtcgcaccaggccgacttcttgccccagaggtttttctcgcccttgaggccctcggcgttggtcagcacgcccttgcccggcttttccgtcaggtcgtcgcgaacccgaaccccgaaggaaccttccttggtgtcctcgaaaacaatcccgccctcgctggcggtgatctccgaggcgacctccatgatccacgatgaggcatcccgggcccggaacacgagggtgcgcgcctcggtgaggatcgtcttgcccgtggaatccttccaggtgttggcggtccgcacggtcacggggccattgccgtttccggtgggcgtggaaatcgccaggccggcgatcgtgccatgcccggtttcctcgctccagaagtcgatgccgtcgatccccttgcgcttgggggcctgataaccctcaagcacaatgtcgccatggcagaaccacgccgatttctggtgcttgtggtccttggcctccttggcgtcgggcaccatcggccaatcgcgggtaatcgccaccccgcccggggtgaccacaggccagaaatacggcttcgcgaccttcgggccggcgttgtaccgggtgacgacggctccacccagcttgaacgaaaccacgccgtcgggcgaaacctcggcagccaccggaacctggccggaaaccgccagcacccacgccgccaaccaaccacatgcactggacatgcccggccctcccggcccaccatagccgtgtgaatccttggatgatgctaacgggtattggttcaagagtcagcagtctcccccttgaggatcgattcatgcgatttcccacatgcctcgggctggtggcccttgccgccgccatggcggcaccggcccgcgccgcggagaccgcgctgccgcccctgttggtgcgcctggcgtcgatcgacacgctcaccaggcagttcttcctgctggcggacaaggttggccgcaaggccgaggccgagcagatgaaaaacgcgctgcaggccgccaccggcccgggcggcatcgagggactcgacccggccaagccgatcggcatctacggccgcatcggacccaacggcctcgacagcaccggcgtgatcctcgttccctccagtgacgaggccgccatgctcaaggccctcaagaaactcgccggcgccatcggcggcaacctcgtggagtcgaaggagaagggcggcgcccacacgctcaacctcgaccagtcgcccatgcccatcttcctcaggttcgccaagggatattgctgggccaccttcaaggacgagtccgccatcaccatcgacaagcttccagacccggcccggctgctcgaggcgcccgccggaagcgtggcccgcctgcatttcgacctggcggcggtgccggagaacctccgcgccaccgcc

SEQ ID NO: 4 - DNA, 13579 bp, unidentified
  misc_feature 1..13579: note = Ga0172382_10009164_organized_organized
  source 1..13579: mol_type = other DNA
ccgtcattctgagcgtcagcgaagaatccagtccgtgattcttaatagattagggatgcacaggatgatgaagatgaaaaagggattaggttttagggttcaaaaaggttgctaactaactgcggattaaagagttggaacaccggattcatccggtcgtaggtgccagattcatctggcacaatgttcttccgaccacccaactctgcccaccagcacccaaacccgcgcccagaccgtcattctgagcgtcagcgaagaatccaggccgtgattcttaatagattagggatgcacaggatgatgaagatgaaaaagggattaggttttagggttcaaaaaggttgctaactaactgcggaataaagagttggaacaccggattcatccggtcgtaggtgccagattcatctggcacaatgtacctctgaccacccaactccgcccaccagcacccaaacccgcgccaacccgtcattctgaacgaagtgaagaatccaggccgtggaaaaagggcttttactatccatccctcttcgctctttaggcagaaaacctgtgcccatgagtgtcatcctggagcaacagcgataggatccagtccctgcagttttacaggccttgtgcgcagcaaaacgctaaaacccacaaccccattatatagaacgagtcccttgaaaaagacccgcaaaaccctaaacctttgtgaaccttatgcttgcctttgaaacataagcgatactaagggcggcaaccaagggcgtacacttaaagtgtgcttacgagaccacataagcccgcaatagcggtattgttcgagcaggcagccaattcacatagtctggtttgccaaaagaacattccttatcaataaggcctggattcttcgctgacgctcagaatgacgggctggtctgggtttgggaggtgaaaaggcgagatggagacctggagacggggagactgtacttcagtagaaagtaaaaagttgtcagttttatggcagtatgcgcagcgaaatcccaaaaccttaaaatctgcgttatcggcgtaatctgcgtgacactctatccgcgtaaaccaaaaaacccgggagacaagctcccgggtccattcttatacctgtatcgccaggaatcagttcttggctttcacatagtagaatttcatgtcggctggagcctgggtccactcggttccggtccagtttccttccagcagtatgtagccgctgtaaggatcgttggagccatatatgtcgtaggcggtggcgccggcaacggggttccagttgagtttgatgttgtttccatccttggtgatggtgaccacaggtgcttccagtccttccgtgtaggcttcgaagtcgctcaggaagcgcggtgagatctgcatggttgcgttgaattgacccgccaggcaggtgatgttcacaggaacagtatgcaccgcagtgttggcatagtctgtattgacgaaagtgcgcattacggcgctgccggaggggtcggtgacattgatgttctgggcagtggcaggataactttgaaacacggtgtcatccacattcacgttcatcaccttgatcagctttgcctggtcggcgctggtgaggcttgccagggtgcgaacttcgggaaccaccacgttgtttgaggaagtggcggcgccggggtcggctttgggcacaaactgcaggagtcccgaatacaaggtcagggaaccgctgatgccggtgatgccgtcgtagaggttgtaggtgctggtgataattccagccaagtcatcaatgacgatggcggcggtggcgtcctggatatacttggtgttgcgattggcgttttgataggtgagcacagcttctcccaccaggcgataaatgtcactggtgtttacaggtttagtgcgcagttcggcgatgtcctgcacatcgatggggaagttgtaaacctttgtggcaaccgtgcttgagatcataccttccgctgtggcatacgccttcaccgtggtgttttgagaaacgttgaagggagccagatacaaagcaccattctggatggggtcgctgccgtcggtggtgtagtagatgtcggcaccgtcagttgcacaggtgatgctgacgttaaacggggcataataggtgcctgaatcaggagaaatctcaggcagttccaccacttcttcaccgggtgcgaagatatgccagcccaaattggtaatcgtatcaatcggatacacgtcccattcggcggcgagggtggggaagccagcgtcggggttttccgtcactccacccgtaacggcggctttgcgcactaaagttttgtccagggtgctataaccgccgtctgcagtccaagctgtgccgggatcttcaccgatggtgccaaagatgtccacataaacttcatcttcgcccacaattttcagcaaagcaagggcatcgttaccattgaagtttgtggttgcgctgttaatggtttttaaagcctgcagcgctgcacccgcttgactgtggacaagcacataaacgctgccatggttcagaattgtgccggattcgaaggtttcggtgctggttggggtggtgcctccattggcaaaaagcttaaccttgtaatttgccaaatccactgcagctccggtgccgttgaagatctcgatggctttgttgttgctgctgccttcgatatattcggagaagaagaggtctgtggcatattggtctcccgagggcgggattgccgttccgcttacgggcagattctcagtgcttgctcccggtgtttcgtggacgatgtttttgctatactgtcccaccacgcttgcattcatgcgcacataaatcgttccattgaagttgttgggcaaggtgagttcatcagtccagtttccggttccggtttcgctgagttcgaaggggctttccacctccacttcaatattggccaatgcgcccgtagcgctgaggctataggattgaaccgcggagggactgccaatcgtttgttcaaaagcatccaggcttgcaacaatgttgaaggtcacatcgggagggaaggtttcgccttccacgcggatgctcaccggagcagcgccggagctggtgtgaacaatactgccggcgtgttcgccaagttcggaactgacgaggcgcacatagatcttgtcatcaaaagtggcgggaacctgaattgggtttccccagccttgttctgcatcataggacacttcaaaaccggtgggagcggtgatggtgatgggtcccaccaggttttcgccgctcaggttataataggaaatttcttccgaaggggttccggcaatgtttgccaaaggatccggctcaccagaaacgtggatgaaacggggttccggggtttcgctgctccagaggagtgcccaatcgttcgtcacacggatgccgtcaaaaatggcatagggagtgtttgcggtttgcctgatggcaattgcgccaattccggtggaaccgatatcggcctcattggtaatagggctcaaagttggagtcggctcgttgggaccgatgacggggttgatccagccggtgacgatatcgtttgtggttccgggcacgaactcatacttcaaaaccacaagataggtggttcccggtgcgtagtcataatctgtataaacaacatttgccccggcattcgattgttttgtgaggccaaaacgaatattgtcgttggcgtctttgcttacaaagaatttggctttaaaatcactggtgctgttggcagccataaagtgatagacatagtctccgtttccggtgctggcatcggtcacgttaatcaggaaagaggtgtaaacttctccgcttgtttggggcacaaaagctctgtttacgtcttcaccatttccaagagtttgacccgcataaccgctggaggggggatagtttggatagatgaggttgatgtctaccacggtgggagaattgttacctgctccgctatgagctgtccagccgttttcaaccaaggtggtgccgacttcatatacaaagttttcttccatgagaaggacgggctcttccggctcttccggttcataaaccgtgccgctcactgccatattaacatctgatgcgccggtgctgctgtgggtgatgtttccggaatattcgccggcggttgtgccaatcaggcgcacataaaccgaaccgttgtagctggatgagagggaaaggctgccgctgtagctgctgccatcgctggaaatttcaaagccggcgggagctgccacgctgatattcgcagtgaggttgctgccggaaaggctgtaagtttgagccagggaagggctgcccacctcagtgctaaaagctgtgaggctgccgcttacggaaataatgggtgtgggggttccgccgctataaccggtccagctcagattatcgattacaagctgtctgtaattacttccagttcctgtagtactcttgattgaaataactacatctcctgggatattcacgttgttaaccgtaaaagtgtggatggtgtcatcagcgcctgaagtgccaccaaaggtttgagaatcggcgacccaatcaccattgacataaagagcaacttggcgatcgcccgcgcctgtgaaagctttccgcatttgcaccgaaaaatcgccaattccgccggggatagaatttgaataaacttcgctttcttttcctaaacttctcagaagcaatcctttgtcatcaattggataactttgttccccagagcagtgaacatagttccaagtgatgttgttatctcccacatagttgccgttaaaataattgcttccggacaacgtgtggttctcaaaagtttccaaaccttgagcccaaacaatgccaagggcaaagatgaggctaaaggcgataagtagttttttgaacattttcatctccttgcttaggttctgataatctcatggcgcggcttttgaggcctgccgccgtttataaaacatcgtaaaataggtccggcatctggcacagagccggatatgataaattataagtcaaagcggctcttttacaagcagccacattttccctatttaggtcaaaatccaccggaggggtttatccgtcaatagaattctggcattttttaggcaaatagcggggctttattttctatcctgcctaaattatggattgcgcgcgcgtgggatgcggattatcatttatgatatgagaaaaatcaattataaggaaaaggaaacacaatgaagggacttggccgggggtggcgaggatggataaaatatttcttgaagataaggagatggtacgacaatgtacattctcattatgtttgccttggcgcctggcttcacatcttacaaatactagaaaattgatgataaaatatatatggaggagagctcaatgtataatttgcttgaacaagattgggtgagggtgctctaccgcagcggagaacctaagctaatctcgctatcgagactgcatcgggatgcggataaattgcatcttgcctacagtaatcccatggaccggctggcggttttccgctttcttctcgctttgggctattggtgttttgccaatacgaaccaaacaccggcacgagataaacccatacctgaagcatggctgtcctggctggaggagaacaagcagtatttgaaactttatggtgacggtcccaggatgtatcaggacccgggcgttaaacgcacacgtcccatcaccgacttgctgcacgaaattcccacgggaaacaatctttcgcatttcaaacacaccttggactatgtctccggcttgtgcctgccctgctgcatacaaggattgctgcgtttccccatttatgcaacctcgggattgccacaactcaaggccagcattaacggcatgcccccattttatgccctgccatgggggaacactctggcagaaactctttacctgaattgggagccacgcgaattgatgggaaccccggcctgggttttcaacaccgttgaccccgaagacggccctgtgccactcttggagggtcttacttcacttgccaggcgctgtgtattacatgaccccatacccggagatggcgcctgttccgcatgtggcgaccgaagcggaagcttggtatattcctgtggttacgacacggctggtgttgttgccaatcccatgtgggaagacccgcacgtatgctacgatgtcaaaaaaaccaaagcaaacaaggcgttgaatttaatctctgaaaaaccaatgatgcaggatagaaactggcaacgtgcattcactggttcttccaagttggagtccggcgatgaaagcatcaatctgctctgcattggagtatcagttaaccaagccgtcataaatgacgtgtgggagtccaccataagaattccgccccgggaagccggaaaagaccccaaaagcataccgaattggacttggattaaccgtcgacttgttggtggatatgaaagaaacagattgtattgggccgataaatcagtttccggaaaacccagggctccgctcagtgaatcgctcatcagcgcagtttctccgcatattcttcattatgcgattcaggagacgggtggttttttgccggataacggtgaattgcccaaatcggtacagattgagtatagaaacatgctgaaagctgttgccaaatcccaggcaccggctgtcacaatagaggatgccatcgcgtccaagtttgtgcataatcagcgtccacagcttgagcaagccaaagcttccaagcaggataaaaaggaggaccaagttgattgaaacatccacagctcaattcatcaaaagaattgcagaactgaagtctggagacctgggtcggctgagacaattgagaggttgtgacctgcatgagaatctggacgggttcgacctgttcaccggcctgtggtggacattgagaaaaaactcgccccgggcgccccgacgtgagattgcctggctcgttaccaaactgtatgcgcagttcccattctctcaagaggacgggctaaccttgcctgtaatccttggacgtatttacaggactctggcaacgcaaaaggaaaaacgaatcttcgccatgaggacggatgccattctgcaagcgcaggcaaaccagttggaacaccccttaagctgggccctgcggatgcttgccctgtatggctacgcttcgctggattggattcaacttaccgacgacctttcggtttggcatgaaatagtgcgacaccgatggacggaggcatttctaacacaatcatcaaaggacaaaaacaaggagaattaaaatgttaatcgaaatgcacatgattcaaaaccacagcccggcaaatctcaaccgggacgaccttggcgcgcccaagacctgttatttcggcggagtgctacgttcacgcatttccagccaatgcatcaaacgcagcatcagaaccagcgatgattttcagactctgcagggcggggttcgcacacgcaggctggtggaacttatccagctggaagctggggacacagattgcaggaaaagagcacaggaaattctgacctattgcggatttaaacatgaaaaagacaacagcaaaatgctgattttcatgtccagagacaaggtgaaataccttgccgagattatcttggatgaatccctggaaatagcagacgctgcccagaaggttgccgatgtgattacccaggccacgtgttcccccgatattgcgctttttggaaggatgttggaacctgaaacaagcaagactaaatggagagatacaacagtggaggcagccatgcaggttgcacacgccctttccacacacgccgcccgtccggaaatcgattattatgtggcggcagatgacgttaagggagaagatgccggtgccggctttgttgacgaagcgatgtttgcatccgcctgcttctataaatatttctccattgattgggaccaactggttaaaaatttgaaaggcaacgaagaactcgccgcacaaacagttggggcatttatcaagtctgctgcgctcaccaatcccagcggcaaacaaaacagtttcgcagcccataacccgcccgatggcattttggtggaattcaaggatacacccgtcagttatgccaatgctttcgttcgtcccgtaaagcttaccgccgaatcggacttggtggaactcagcataggccaactcgccaactatgttaaagatgtaagcgaagcatattatcccggccacatggaaggcaacgcattttggttctctcccaacggacgccatcccttgttaccacaagacggggacatacaagccgagcaagttggacaactggaagaactgataaagaaaacggttcataaatgcggttatgagtgggaagccatcaagcaaaaaaaagtagaaggctgatatgagcgccaacaccctattcctaagactggagggaccacttcaatcctggggcgggcatgaggcgaaattttccattcgccgcacttctgatgctcccaccaaatcaggagtctttggcttgctttgcgccgcactgggaattggcagagtcgaagcaggtgaaagctgcctgcccgaactggcgaacttacgcatgggagtgcgcatagacaggctcggtgtccgctggtgggattaccacaccgtgaacgctgggattccgatgcttaaatctgggctagaggcgtctgaagacctcagcgatgtgggcaaagccagggcagaccgactgcttaaaaaagaactgagtttcgacaaacgcgggttgctcagccgtcgcgaatatctggcggatgccagttttctggtggccttgcagggtgaaccttcaacagttcaaaaactggcggaagctctgcaaaaacctgtctggacactatatctgggcagaaagacctgcacaccatcacgccctgtgggagagcatacccccggtaaatatgcgagccttgaggaagcattgttggccatacccgtgcaggtgagggataaacacgaaaccctgccccagtccctccaatgtgttctggactggattccggcttatgatggtgaacccgctccggattgcgcggaaatccactacgacctacccatgtcttttgaccctccccggcatttgccgagatttgtgattcgccgtgaactgcccttaagtgtagatatcgaaacacaacaggttatcattggaagaccctggcgtccggcacgcagtagggccgattacacaaacactcaatataagagagtccgcgcagaacgtctggtgatggatgtcgggctatgcatggtatgcaaggcgcctgccacaacagtgcaacacgtaaactatcgtcgcactggagggagggaagagcctgaagatttgcgcgccctgtgtcggctgtgccacgacgcatgcaccatgctggaatatggcagcggcatgaccatggaccgcatagacccgtgtgaccccgcctggcgtaaccgcatcataagcaaacgcaatgaaatcatcgaattccgctcgctggaacaacgcagacgaaagcttgaccccaaggaggaaaacggatgatgtatctttctcaattactcatagataccggtggaaatccggacagactcagaccgggacgttcgtggctcaacaatatttacaacgtccaccgcaggctttccatggcctttcccatgcggcaacgaaaagatggtgacccccggttcctgcagccttttaacccggctgactttgagcgtcaccgctttctgttcagggtggacaacaacatcgacggagaggaaaccagagccatggttctggtccagtcatcaatactgccggattggaactacgccttccaaaacgccaccggctttttggcggctcctcctcaggtaaaagaatacaccccaaatttttcccccggacagcagctgcgcttccgcatcctcatcaacgccagcgtaaagcgcaaaatcgaagaaatgaaagacggggactccggaagagttaagacggggaaagtgctgcataagcgtgttgccttcacttgggatgctgatgtttccccttccgatgcacttgccgaatggttcgccaaaaaggcgtccaaactggggtttacgccaattaaaacagagctggttaagcttggctggatatatggctccaaacctgaaccgcgaggtgaaagaccacgaagagaaaatgaataccgccccctgaaatatcgttctgcactgctggaaggatatcttaaagtggatgagccggagttattcaagaagactttggtcacgggaattggcagtgcaaaaagcatgggatttgggctactttccgtaattcccacacaagatgcgtaacaaagacctgcgctccatcccaaagatgagagaccgcatcacctacctgtatgtggaacatgcaaaggtggataaaaaacataaatccatagcctttcatgatgccatgggttccacacaggtgccatgtgctggactggctcttttgatgctcgggcccggcgtatccattactcatgccgccataatggtgttggccgaatgtggttgtcttgttgcctgggtgggtgaacaaggcgttcggttttatgcttcaggcgccggcaccacccgcagttctgcaaacctgatgcgtcaagctgccgcatgggctaatcaggataaacatatgaaaatagtgcgcacgatgtacgagatgaggttcatggaaaaactggacacctcatatacccttgcccagattaggggtaaggagggtgcgagagtgcgagacacatatttcaggttgtccaaagaatatggagtcaccatgaaggccagagaataccaaagagacaattggattaaaacagaccctgtcaaccgtgcaatttccgtggcaaactcgtgcctatacggaatatgtcatgctgcaatagtggctgcgggttattctactgccttgggctttatccatacgggaaaacaactctcgttcgtatacgatattgcagacctttataagatggaaacttcagtgcccgctgcctttatggcaacagctgaaggaagtgacagccttgaccggagaacgcgaatagcctgtcgtgaccaattccatcaatttggtttgctgaaaaaaatcgtcgccgacatccatgcccttttcgacattttggaaactccggatgatgaacccgacctggtggatatcgatgccgcagcacccggtgggctgtggaacccggataaaagtataagtccaggcgggcaatcatggggtttggattcacaaggaactgaagatgatggttctgattcttgaaaatgttccaatctcactccgtggtgaaatcacccgttggttgcttgaaataaaagcaggcgtatttgtcggcaacgtgtcagctctggtcagagaaaagatatgggataaagtcaggtcagattgtgccggcggcacttgcctcatggtcttcagtaaacattgcgaacaaggctttgacataaagttttggggcgctgcaggaaaaattgtcacagaatgggaaggtttaaaacttatgactaaacatccgtaaacgatacttgacatagctccaacagcagaaaaccctttatttacatagtgtattccccacgtgtgtgggggtgaaccgacaaatggtaaagccattttttctgtaggtaagtattccccacgtgtgtgggggtgaaccgactttatcgaattttttgagagccattatttagtattccccacgtgtgtgggggtgaaccgtatgacccaatgaccacgagccaggagttgaagtattccccacgtgtgtgggggtgaaccgacagaagcgttatcaataccaataattctgccgtattccccacgtgtgtgggggtgaaccggcaagacgggagcttatgtgcttgtctgtcgtgtattccccacgtgtgtgggggtgaaccggcaggatgacgggcaggtgatggatatatatggtattccccacgtgtgtgggggtgaaccggaactggaaatcgaagccatccaagccaacctgtattccccacgtgtgtgggggtgaaccgcaaagcattgcggaaatcaaggcgctggacttgtattccccacgtgtgtgggggtgaaccgaacgcaacaaaataagccagcacgcgccttgtgtattccccacgtgtgtgggggtgaaccgccagtatctccctcagctgcccatacttaattgtattccccacgtgtgtgggggtgaaccggtccttaggggtgcgcaagtaaggcaggattggtattccccacgtgtgtgggggtgaaccggatcaggtggacgtgaaagcgctgttgaagctgtattccccacgtgtgtgggggtgaaccaagcaagaaactgagcgggacgcagctcaaggcgtattccccacgtgtgtgggggtgaaccgataagagaccgttgctgtcagacctgagagaagtattccccacgtgtgtgggggtgaaccgaagcatttgatacgtcacctcataggcaggcagtattccccacgtgtgtgggggtgaaccgtgggttcactacctgaaagaaaagaacaaagagtattccccacgtgtgtgggggtgaaccggtgtccgggctatagccctctttggtgatgctgtattccccacgtgtgtgggggtgaaccgcgttcggctccttcggtccccgtggcggcttggtattccccacgtgtgtgggggtgaaccgctggcataattaagtttgtgctgttccgatatgtattccccacgtgtgtgggggtgaaccgatttggagccgcagacgccggatgcgggtgcggtattccccacgtgtgtgggggtgaaccgctgcctccctcgcttgccaaggccagggcggggtatttccccacgtgtgtgggggtgaaccgcagggctcgaccaatgagcaagaccaagcagagtattccccacgtgtgtgggggtgaaccgaactgtatggggacggatacgccctcaagcacgtattccccacgtgtgtgggggtgaaccggaaatccgccaggattatacgctgaccagtgggtattccccacgtgtgtgggggtgaaccgaaaaagaaaagaaaaagaagagctgcgctcaagtattccccacgtgtgtgggggtgaaccgtggctgaacggggattggaatgataacttgccgtattccccacgtgtgtgggggtgaaccggacgccccgccttgggcaaattggctggcgaggtattccccacgtgtgtgggggtgaaccggcgagggtggtgatgcgggccgcggcgttgcggtattccccacgttgtgggggtgaaccgaggtaaatgaaggtggaaacctataccgcaagtgtgccccactctcgtgtggctcttcaaggcattcccccaaaaaagtggggaattgtagttgtagcagtatgtttatcagatagatagcttatatttctgtccattaatttgtgttttcggatgcaaatcgggttgtctatgcaca

SEQ ID NO: 5 - DNA, 12676 bp, unidentified
  misc_feature 1..12676: note = Ga0187892_10009586_organized_organized
  source 1..12676: mol_type = other DNA
agggcgcgcgatcgtagccagggcattcatgccctgctggccaacgcgccccgaaaccgcccgcccggagggcggacgagggttagtgcgacaaggttgggggcgcctgtttttcaggacaggaccgttgcttgttgtcaatgagtcaggttcgaatcgcgtccgtagccccgaaggagcgacgggttttagccacgggtgcagaccccggaggggtcaaacccgtggcaaccaaagcgccggaacggacggagccccggcaggggcgaaggagcggcgagtctgcgtcaggtcggatcgggaggcgggccggagagaccggcacatccctggctggtctgacgtgcggcttctcggaggtgcgcctcactagacttcttgggttgccggacttgttgcgaatgagtcaagttttcgagtcgcgtccgcagccccgtaggggccacggcatttagccgcgggtgcagaccccggaggggtccaacccgtggacatcgggcaccagacgaaggtagccccgaaggggcgaaggaaaggcgagtccgtgtcagatcaacttccgggaggcgtcgcagggcttccgccgccccttttggggctgatggggttgacgggcattgccatccacgggcttgaccccgctggggtttgcgcccgtggctacaacccttgggccctgcggggccgttggatttccgcgcgcgactggttggtctgaggcgcagcctcgctagaattcccagacagcaaacactcgccggcagcgatgtcggcggtacgacgtgatcgaccccgtactcatcaccgcagacgacaagcatcgggccgccccggtacgcctgttctcgccgcagtatcgccggctgctcgggttcctgtggccccaccggcgcgccctggtcatcggggcgatcgcctcggtcctgcacgccctgctgaacacggccgggatcagcgtcgtgctgccgatcaccacgatcctgctcgaagagcagggactgcacggctgggtctaccgctcgatggccgagcggcggctcggggccgatctcgacacgatctcacggccgcaacccgatggaaacgtctcgatccatcgcgtgcagatcctcaaggttcgacggggttcgccactcgacgcggccgatgtcagcccggcctccgagaccgacacgctggacggcgagcagctcgcacccatcgaatggttccggcgcgtcgcgctcacaccgcaagacgcgaccgtcacgttgggcgtcatgccatccgggtcggccgatggcagccctgtttcggcctcggttcggctgcgcgacgcacccgcgcactggcgggccgcggcatcggccgtcggactccttccattcggccagccgctgcgcgcgttgctggccgtgatgggcgtgctggtggtcgtggcgatcgccagcaacatcttccgattcttgtccgagtatctggtctcgaccgcgatcctgcgctcgctgatggatctgcggcgaaagttgtacgccaaggtcctgcggctgccgatggccttcttcgcccgcgacgtcggcgataccgtctcgcgcttcgttcaggacgtgcacgacgtgcagcgcggcctgatggccctgttcggcaacatgatccgcgagccgctcaaggccctggccatcttcacttgggccttgttcctcgactggcgcatcacgctgacgctgctgctggtcggaccgctggccatcctgctcttctggcggatcggcgagagcgtcaagaaggccagcacccggctgctgcgcggctacggaatgatcatcggcgccctgtcggcctcgctgcacacggccaacatcgtcaaggcctacacggccgaaaacaccgagcggcgccgcttctggcgcatcgaccgcaagatgctggcccagcagaagaagatggtccggctcgacgcgctgctcagcccatcgctcgaggtcatcgccgtcgcctcgatctgcgggctgaccatctggctgggcgcccaggtcatcggcggcacgctcgaccagccgcgcttcgtggccctgatcgtcacgctggccatgatgttcgacccgatccgcaaggtggccgacgtctacacccgcgtgcagcgctcggccgccggcgcgtcgcgcatcttcgacgtgtatgaccggcccgacgaaacgtcgcaaaacggcaccgcgcccaagctggccccgctggccgagcgcatcgagttccgcgacgtcaccttcgtctatccgcacgccgatcggccggccctcgaccacgtcagcctcagcgtgcacaagggcgagaccgtggccatcgtcggccccaacggctcgggcaagaccacgctgatgagcatgctgctgcgcttctacgacccgctggaaggcgccgtccgcttcgacggcgtcgatctgcgcaacgtgagcctgcgcagcctgcgccgccagatcagcctggtaacgcaggaggcagtcgtcttcgccgcgccgatcgccgagaacatcgcctatggcgcccgcggcgcgacccgcgaacagatcatcgacgccgctggccgcgcctacgccgccgacttcatcaatcgcctgcccaagggttacgatactgtcgtcggtgagcgcggcgcgacgctctcgggcggtgagcgtcagcgtttggccatcgcccgcgccatcctgcgcgacgccccgatcctgatcttcgacgaagccaccagtcaggtcgattccgagagcgagcagaagatccaggccgccctgcgcgagctggtggctgaccgcaccacgctgatcgtcgcccaccgcctcagcaccatcaagttcgccagccgcatcgtcgtcatggacgccggccgcatcatcgacacaggttcccacgagcagttgctggcccgctgccccttctaccacggcctgtgcgaaacccaactcgtccactagccgccgttccgatcagaaccccggccgccaaggccgcgggcccggatcggatcagaaccccggccgccaaggccgcgggcccggatcggattagaaccccggccgccaaggccgcgggcccggatcggattagaaccccggccgccaaggccgcgggcccggatcggatcagaaccccggccgccaaggccgcgggcccggatgccggtagcgcttcgagttcggaccgagcgtactcctcggaggggctgcagactgctcgtgttgattcgcggcgcgtcccggacgaccgtcacggagcttcactacagccaccggacccctgacccctcgcttgcggtcagaagggccacggatcaacccggacgaacaccggcgcccgttccggctcccggccttcagctttcagctttcagctttcgctaccgatcaacaccggccggcctccaggcaaccctcaagcgcgccacgccctcccccagcccgtgattggttcgccttgcgtcaatcgcccgcccgtgcataatgtccgcgcggtccggtcggaggatggatcaagtttcacgcgtacgctgatcaacaccgcctttcgcccctcagtgcctgctcgagtgacgatccaggcgcgtcgcgctctcgcgtgaagaaggacgcacgggtgaccgatgactttcaatttgctcgttgaaccgtggattcccgtcctccgtacaaatggacgccctgcgcgagtaggcgtccgggaagcgctaacgactgcgggcgccatccgacagatcgccgcgtcgaacccaatggataacgtcgctctgctccggtttttgctggccgtgctcatgtggtgcaagccgcgtctaagtgacgaagaacgcacacagctcgacgtcgcgaagggcgtcccagaagagtggctggccgaacgactcggcggtgcaagtacgccgtgcgaggagttcaacctgctcggtgacggcaaccggttcttccaggatcgatctaccccaaataacgcgcgccccatcggcgacctgctcgttgagtttccgacagaaaccaagatcgcgcgcttccgccacgtccgagacacgcagtacggcttctgtccggcctgcagcgcgctcggcgttgtgcggttctgcgctttcgccaacgcgtacggcggcggtcgctacacatccgcccttaacgggcctacgcccgcatatgtgatctcgcaccgccccacgctagttggcactctgctgctacactggcctatgaacaatccggtcaaacgatcgccgccgtggctttgtgctgatccgccgtccgaaagcgacctagaccttcccacggccttcgcctggcgctcgcgccgcatttggctaaacgatcccgatgccgacggggattgcgcatactgtggtcaccgaagcacactcattcgacaactcgcgttcaccggggcgtggaagccgccatttgaaaccaaggggcaggagaagaggttctgggagaaggatccgcatctgatcgtcgtggagaaggaccgcgaggcgccagcggaggagacggcagccgatgacagtgacgacgcgccccaccgtgacgccaagagcagcgagcgccgccgcacgagaacaacgctcggtttcccttccccggggtcgcgagtcgcggcacattcgaggttctggcgcagagcccttcgcgctaggatcgggcacgccagtggcaaaaacgcggacgatgcaaccgctgtgatcgttgcgggacccgctgccaacaaagggctttatcaggatgctagcgaagtccgcttgccgcgtctttctaacgatgacggcaccgccgcgtcgattgatgctctggcgaaggcggttgatgctctgagcgccgtgttgaagtgctccacacgcaacccggagcgtcagcaccccgagcacaaggccgcgctcgattcgttggcaccttcgctagaactgggttttcgcgaggatactcaagcgttactgatcccgagcttctcaatcgatcacgcgacagagcgactcttacccgtagtgaataccgtccttcgcgccacgtccgcaggctcgccgctgcgccggcgcgagggagaggcgcgcgcacgcgaggcgttgaatcgcgttctccactcggtcgctgccgcgagcggttcgcccgcgaaatatcaaaatgagcccagcgcgggcgaacaaagggcccgtgaacacaagcgcacgcgcgcgacagagtctaggagaaccaacgcgtgagtgcgacggagacctacatcgcgcggctgctggccttgcaagaggccaagtgctcgctgctgcgcggcctcgcgggacgccggctcgatgattcgctcgcggggttcgatctcttcaccggactgtggtggccgttgcgtgccgctagcccggccgtgccccggcgcgagccgagttggctcgtggccaaactccatggcgccgcgcgtgtcccgcacgtgcggccggagcggggattaggccccttgcttccggccgtcctggccgtttgcgagccgtcggacccgccggaatacaaggcccgggaccgcttccgcaagcgcttcgacgccctgctctcctcgccgctggccgctttggagccgcacctgcgctgggcgctcgcagaggttggccgggcggttcacggccgagttccagaggcccgccgcgtacagggcatcgactgggcccgcttgttggacgagctgtcaatttgggaccgcggcgcccaacaccgattcgaccgcgatattcgggatatctgggccgaggcgtaccttaaggcggccgacgcgcggcgcgcctgagagaaggagcaacgcatgctgatcgaaatccacatgatccagaaccacagcccgtcaaacctcaatcgcgacgacttaggggcacccaagacctgcctcttcggcggcgttacccgcgcacgaatctcgagccagtgcctcaaacgcagcatccgacgcagcgcggagtttcagcgcgctttggagaaaggcggcggggtccggacgcggcgcttggtcgcaattctgtcggagcgcgcgcacggctcgccctccgcaccccacgacttgatcgagctggtgactaaggcgttcgaaaagggtggggtgaagaaggatcccacgaaggacgacccgcacgcaacgaacattttgttgttcgtgccagaggggacggtagtcgcgatggcgaaagcggtcaaggctgagtacggcaagcccgcggctcaacggacggccgatggcttggctgagacactgacggaactcttcggtcagaaggcgtcagtgccagacattgcgctttgcggacgcatgaccgaacttgactcagacggcctgtttcgcaagatcgactttcgcgttgaggcggcgcttgcagcggctcacgccatctcaacgcacgaggccatcaacgaggtcgattacttcacggctgtcgatgaccaggcggtcgggaccggtgccgggcatgtcaacgaggccatgttcgcatcggcctgcttctacaaacacttctgcatcgactggaaccaacttctcgacaacctcggcggtgacgagccgctggcgagtacgaccgtgtgccagttcctgcgcgctgcggccatgaatgtgccgagcgggaagcaaaagagctttgcggccttcaatccgccggacggcattctagttgaagtcaagcacgaacaccgcctgcccattagctacgcgaacgcgttcgctgatcccgtcccgctcaagactctgcgcggactgattggcgaaagcatcgcgcgcctggggcagcatgttcacgacattacggccgggtacggagtcaaagcgaagcggttctggttttctccggccgggcgccaccggctgacgtggattgaccgaacggctaaggagggtgaaaaggagaagctggccatagacggcgaagatggcacttttgattcgttcgacaaattcgtcagcgccgtaatgcaggaactgggaaggaccgcctcgtgactggaagtaccgttttcatacggctggaagggccactacaatcgtggggcgacacgtcgaagttcgtgattcgccgaagcatggaggccccgacgaaatccggcgtccttgggctgatctgttgcgccatgggtctttcgcgagaggccgcgcgcgaggtgctcacagacctgaataccttggttatgggcgtccgtatcgatcgggcgggcatgcgttggtgggactttcacaccgtcggtgccaaggtcggcattctgaatgcaaagggccacgtgaagaagacggccagtacgggcgagtatgaaacactcatcacccggcgcgagtatctcgccgacgcgagctttctcgttgcactgcaaggcgacgatgagctgactaagatcgtgcacgctgcgctcgagtcgcccaagtggccgattttccttggccgcaagtgctgcgcgccaagcgtgccgctactcgctcctccgtcggacggcgaatcctggacgaacccgcaggtacacggcagcttgagcgctgccctcgccgccgtatcctggcgccctcgatacgattccgaatcgccgcccatacctacgacgcgcgacggaagggcggagcttgattgcttgatggagtggcgggccctaagcgacagcgacgtcgcgcccgatgcggccgaggtatggtacgatgtaccggtctgtttcgatccccccgtccacgagccgcggatcgtcacgcgaagcactacggtagtcccgatcgcgcgagcgctccaacaacacactccgccgccgccgcggccgcgcgctgattacaccagcagagagtacaagaagcaacgcgcggcccgtcttgaggctgatggcggtgtgtgcgtcttctgcaaatcgcccgccagcaccgtccagcacgtaagctaccgtcgcgcgggcggcgctgagacgcaggacgacctccgggcgctttgccggctgtgtcacgatgccgtcacgatgatcgaatacggcctgggcatgggcctcgaccggatcgacccgtgccaaccccgttggcgccaagacattctgcgtactcgggccgaaatcgtgcgtttccgcgcgcttcaagtacggcgccgacgccttagccccgacgaggtcgaatgaccatgtttctatcgtcgctcctgatcgacaccggcgaaaaccccgaccggccgcgtcccggacgactgtggctgcgcaacatgtaccgcgtacaccagcgactttgcatggcgtttccgactgcagcgcgcagcgaaaacgatccgcagttcgtacaaccctaccagcgggacgacttcattcacgtgcacggatgtcgcacgaacgatcaggcttttctctttcgtgttgattctctcgccggcggcagagtagttgttctgatccagtcggcgatcaagccggattgggattacggattccataatgcgcagcaccttctcgccgcgccacccgccatcaagttgtttgatccgcagctcgcggccgggcaacgcgctcgatttcggctgcaggccaatcctaccaaacgcctgcgccgcgaatcgccaaacgctagcggtgatccgacaccgcaacagtgggtgggcaagcgcgtcgccgtgcccgctgacaagctccaggactggctcgcgcgccgcgcagagagctacggattccacttgcgcgaattgacggagttgtgtccgggatacgtgtacgttagtcgcactccagagtccgccggcggtctacggctgcgatctgtcaggtacgagggtctgctggaagtcactgactccgagtcgctggagcgtgccatcatccgtggcatcggccccgccaaagccttcggcttcggcttgctctcgctggcgccggtccggtaacgatgcttccccccgcacggcgccgcgcggaactcagcgcagccccgcggcgggccgatctttgacaagtgaataggcgagaatcccggccggaggcgcgccatggacgatctgcgctcgctgccccgcttcgacgatagcctcagctatctctatctcgagcatgcccgcatcgagcaggacgacaaggccatcgccttctacgacaaggccggcctcacgcccgtgcccatcgcggcgctggcgctgctgatgctgggccccggcaccaccatcacgcacgcggccatcaaggctctggccgacaacaactgcaccgtcgcctgggtcggcgaggacggcttgcgcttctacgccgccggcaccggcggcacgcgcggcagtgccgccctcctgcgccaggcccggctgatgagccatccgcagttgcgcctggcggtcgtgatgcgcatgtaccgaaagcgcttccccggcggactcgacccgaacctgacgttgcaacagatccgcggcatggagggcatccgcgtccgcgaggcttacgccgccgccagcaaggctaccggcgtgccctggaccgggcggaactacaagcgtggcgactgggccgctgccgaccccgtgaaccgcgcactgtcgaccggcaacgcttgcctttacggcctggttcaggccgccctcctgtcggccggctactcgcccgccctcggcttcatccacaccggcaagcagctctcgttcgtctacgacatcgccgacctgtacaaggccgacctggtcgtgccggccgccttcgaaaccgccggctggcaatgcccgtccgtcgaacgccagatgcgcatcgcctgccgcgccaagttcaaggacgctcgcctcatgcagcgcattctgcccgacatcgccgagctcctcgcgctcgagggcttgcccagcgtcgccgacgacccggcctgggacgttgaatcggccgcgcccggcgcgctctgggacccggacggccgcctcgaagggggcctcaactatggtactgatgttgcttgaacgcgtcgccccttctgtgcgcggcgaactcacccgctggctcatccagcctcgcaccggcgtcttcgtcgggaccgtctccgccctcgtccgcgacaagctttgggacaaggtcgtacactccctacaggctgccaaaccccgaaaaagcggaaagcgcgccggcgcgattatggtctataccaccaataccgagcagggcttcgacatccgcgtcttcggccaaaccgatcgatccatcgaagacttcgagggcctcatccttgcaaaaaccatcgcccaaccctaaatacgcagcgttgtccccacgcacgtgggggtgaaccgccaaacatgccgccctgccctcgtatagcccggttgtccccacgcacgtgggggtgaaccgagtctcctggctcgccgggcgcgctgtccgatgttgtccccacgcacgtgggggtgaaccgaagattcgggggttattttagaaataactgagaagttgtccccacgcacgtgggggtgaaccgctagacttgggtagtattgttacgcagggtgcagttgtccccacgcacgtgggggtgaaccgactcatgggcacacaccgcgctatcccgatttgttgtccccacgcacgtgggggtgaaccgcaaagcaattaaattgccaatctttaatttttcgttgtccccacgcacgtgggggtgaaccgcggtgctgaccaggtggtgcatgacgagtcgcgttgtccccacgcacgtgggggtgaaccgttgtgaactgtgcctgctcgcagtcagtaacgagttgtccccacgcacgtgggggtgaaccgataaattattggcatcaacgcatccatatgtaacgttgtccccacgcacgtgggggtgaaccgatgctcgtcgaaccggcccacggtgctacgaagttgtccccacgcacgtgggggtgaaccgctcaaagtgcgactcaccgtgcaaggcggcccgttgtccccacgcacgtgggggtgaaccaagatacactattgatagctcccgcccccacatccgttgtccccacgcacgtgggggtgaaccgcaataagctaagttctccgccggaattttagcgttgtccccacgcacgtgggggtgaaccgaattctgggaaatagctaagagaaatctttagttgtccccacgcacgtgggggtgaaccgtggatagcctcggactgggcgatgttcttggcgttgtccccacgcacgtgggggtgaaccgagtattgggccaagagagagctccggaataggcgttgtccccacgcacgtgggggtgaaccgagtattgggccaagagagagctccggaataggcgttgtccccacgcacgtgggggtgaaccgacctgcatcatgtgcgtctgcgatgcgtccggcgttgtccccacgcacgtgggggtgaatgtaaccgatcgggattgtgtgcggcttgtttcgagttgtccccacgcacgtgggggtgaaccgaaactgacggatgaggagaaggcgcagaaagaagttgtccccacgcacgtgggggtgaaccggcgctgcttgcgattgccgcagcggctcgcaagcgttgtccccacgcacgtgggggtgaaccggctacttggccggccggaagccgccccgcaatcgttgtccccacgcacgtgggggtgaaccggtaatcaacccacccacacgcccagtgcgaaaagttgtccccacgcacgtgggggtgaaccgtccagtacgtcgtacgagacgaacatggccgctgttgtccccacgcacgtgggggtgaaccgaaaaacaatcgcttgcactccgggcgatagatagttgtccccacgcacgtgggggtgaaccgcgtaacggtttgcagaccgcgcgtggttgcaaccgttgtccccacgcacgtgggggtgaaccgatagattaagggcatcgaaatatcaatttaagcgttgtccccacgcacgtgggggtgaaccgtcctcggtccccttgacgacggccggcttgacggttgtccccacgcacgtgggggtgaaccgtcctctgagtccggccgcagacgccagccggccgttgtccccacgcacgtgggggtgaaccgttgctcgcactcgggaccgactcacaactggccgttgtccccacgcacgtgggggtgaaccgagccagtttcgcctgttggtccgtcaatgcagcgttgtccccacgcacgtgggggtgaaccggccagcactgcgacgtatccgggcagggctgggttgtccccacgcacgtgggggtgaaccgagcagggtgagcagagttcagagatcagagttcgttgtccccacgcacgtgggggtgaacccggcttcgggcgtgttcccatatcgctgcgcctcgttgtccccacgcacgtgggggtgaaccggatctctcaaaatgagaaaaacaatggatgcagttgtccccacgcacgtgggggtgaaccggaggacgtccgcgacctcgaggacatcagggcgttgtccccacgcacgtgggggtgaaccggcgtgtgcgctagaagccgtctgcgcgtggcgttgtccccacgcacgtgggggtgaaccggactctccggcattttttcgagatttttttgccgttgtccccacgcacgtgggggtgaacccagggctatcgcatctaaaccgcggttagtcttgccggacggtcgcgttcgggttacactggcaaactgagcgtgtgaggcctcacggagtttcttgaacaaacccg

SEQ ID NO: 6 - DNA, 15169 bp, unidentified
  misc_feature 1..15169: note = Ga0315288_10006183_organized_organized
  source 1..15169: mol_type = other DNA
ggacagtcggatttccacgcacccattctactacctcatatcgccatcgcatacattcacccacgggcacagcccgtggaactctgcccttcgggttctttagacgctgtggtaacaaatcgtcaaaacgttaccggatgtcgtgccgcgtggttgaacagacctgtttctctgccccattgaagaaccttcggtcaggttcctgtcccggggcaaatccgcacgatgacggcactggcttttagggatgattcaattcagagcccgagcatctcgttttccggtcctgggggttctccctccctggctgcctgaatttggcggttgatttcggggggaacatcgagaccaagctgtttgaaccgcttctctgtccatcgaccgacgttacgcgggtccttgtagtagtcatgtcggggtgttagtgtgggttctcctgcttcctccagtacgtcgtttgccgaccggtggtaggcgaaggaggacatgacccgctcgatcttgccacggccgcactccggacagttatgcgatggcgatgccgaataggtgcgaaaaaggacgctgaacttcctatcgcatcctttacagcgaaactcatatatgggcatggctatcgtccgcccctatctcaatctttcggcgccttgaagggccagcgtctcacttacgccgtcttcgatcatagaagcgcgggcgtcccggaacaacttctccaccaggaaatccttgctcaggccatacccgccgaaaagctggatagccatgctggcaacacggaaggctgtttccgtacccaatatcttgactgcaatagaatactgaagcgccccaggtggattggcagagttgtatgcggacacgcgccgactcagcgaacgtgccgcctcgacctgaatgaacatatcgaacaaccttgctttcacgctctggtgctcgaagatgactttgccgccctgtaccctctgcatgcagtactggtacgcctcatcgaacgcggcctgagcgcagccgattgcaggcaagcccgtaccggcagtcgtgcccgtgagcagtgattcgtactgcgccggatacgtcttcgggtctttccagaccatgtattcttcaggtatgtgcacatcatcgaagaatatctctccctggttgagcgacctctgtcccagcttgttcagtggcttgcctcgcgagactccaggcctgtccaggggaacgaccgccacgccacagtttgtaagcccgagggcgggttcgatggctacaaacagcgccgcatgactggccagcgttccattagatacccaggcagacttctgaccgttgatgatgtattctttgcccactttttttgcgcggacctggggcgtaaccctggggctggacaccgcttccccggcggagtacaaccagtctgagccgtggtccggctctgtgatagcccaacacccggtgtatttcgcgtcattatcctttatatagttcatgccaagctccaccagggccggcacgcccgaccccagtcccatgaagtacgggaacccgtggcacccgaggctgatacaaagaccgaggtcggcatagccctcctcttcagtcaacaggttacgggtcagcgggtcgagttccagcccgccaagtgcttttgagaaaccggtgcggtggtacccctgtcggaaagtctcccgcatgacatcccagtaaatcgaattcttggcgatgacgtcctgggggtcaggcatcctgtctagcttgatgcccgcgggcctccacacctctttgaagaatttcctcgcctccctgcggatagtcttctgttcatctgtcaggtcgacattaagttccagcaaacccatagcgctcctccttcaaaatgctttgattgacgtgcacgcgaacaccgtgcaggagaatggtaccaagtatggtgtgacctgtcaatttcggcattggcccctcttatccacctagaatccgtaatgttttggggccaagtcaacgccggtccgaaacgtctctgcatatttgagcatcgcaatcgcctcacgtcatagacttggccagaaataggctgcagaaatatcgcaaaatgactgaaagtgactcaacaattcatcgtttcgttcactcgccgcaccgcaaaatggcggaataggcgactttccagccaccttaaatatcgtcttttgcagaacgaccagcggtaaaatagtggtaaaactcgtgatttttcgcaatcgattgctggctctctcttgttaacggtaaataatgtgtaaaacgtacccttctaagcacgtaataacaagctatcgaatcaaagatcttgcgtgtcgcgctttgcatgaccaaggtgaagacgagacggtcctgacttggctagattgaaagccaattcaaaggtcttggttctgcgtattgtacgctgtagcccaccaaaggttacttttgttaaaccgttttccacccccttttttaaatcatcattcaagtttaatcgacaactgattcgacgtgttttaagatcttcactgggaacgacagttacccccagtcgggcaattagctcttgtttatcctcgaagctagcatattccaggttttgcgaacgtaaagataaaagatccttacggacagattccggattgaaattctccctcatgtacatgtcattgatgtgctctatttcttgctcggcactggcaattgtttgtctcaattctttaactttgatccccaattccgcgtccgaatagacgcccttttcccaaccgtcctgtatccttgataacctctggttagattgcttgattttgttttcttccagccgaattaatttatctttgtcctgaagccgattttgttcttcgcctagctgtgcctgcagccacgtatcatcgtgcagcaagtggcatacttcttcccagacactatcatcccaggttgcaggaatgaatttccggtagttgcagggatcctttatccaggaacaatagtgagcccggcaataatagaatacctcgtcactgccagcttctttgcgcattacagacattggtttattacactttgggcaaagtatccgggtgcgcagtaatgctttaatggatttcccctgtttacctctgcccctccctctttctttgatgctctggtttgccagacgccagagctcttctgtggtgagagcgggtacattgtaaggtattctttccttttccggtttggggcgtattagcgttcgtttaacacccatagtagggtcaccgaaaggacgctctgggttattgaagaggccgttgacgttatactccgccttgcctgtgtaacacttgcgattgactattttaatcaccgtacggggcatccacgtttcacgttgcggtggtgggattcttagtttatttaattcagcggccacccaataacaagttcgcccctgttctccagtccagatgaaaatgttgtgcactacccaagccggtgaattggggaaaagactaccatcgatatcagccttctcaatttgccatttagcactatttaccttaaccctgccggtacgctggtctataaccttatccgcaatcatagtgtatccataaggcacccggtgacagggtacttttccattcataacacgcgagatgttaccggcaagcacattatcccggttggactttactctgagcgagttggcttgggcttgaatcagtctggtagtctggctcccccagtcgttaccggtgggggagtcgccgcagacaagcttgaccccatagtgaatgcattcacgttcgagggtaatctggtgcaatgggtcagctgaaagtctcccctgaaatgggacaataactccggaaatttttctgcccgtaataaggtcatgtcgcaaatacttcatgccgggacggtcaaagttttcactcgaccttgcatcataaataatatattcacgaggcacaatcgctccgtattcttttgccaacttagcacaggttagaaagtattctccgagacggtcattttcagattgctctctggtggattgcctgttataggctacccaccaatcccgttttgtgagatcgcacccagtaacaccaaagccttcttcataccctgcgagacgggctgcttctttaatgtagttgttgacacgttcttcgctaataatttctatctggcttaccatcttcgtttctccttttaaaaatcagttctagtatttttttctttgcttcggcgtcaacttcaggtgaaattggttcataaacaaatacactttgtgtctggttttgctggttggtcctctggttcaaaatgatctttgccagatcattcacgagcgtcccgtccaaggcgctatcaatattaaggagaatatttcttctggcaagttcctttaaagcgcgagattgtgtcctctcattgaaaaacagtcccttttctttcagttcggcccggagtttggcattgttgtcatccggtaccccactttcccagccatacttctttgccattatctcgataacagctccgaataatggtttctgcaaagacactttagcggcaggattaacagaaaatgcctcctccatttttgttaccacgtcaccgatcagcagcttgcagcttaagctgaagatttcatctggtaagtcaatatccgacatggtacctatattcagaatgaagtccctcatctgttcaactaaagcatccttcctatcctgtggcgtttctgctaacaagataagtaataaattgttcataaatttaaaggcggctccggtttgatctttctcgggatcttgatttgctccaacattgccttgctcagcaaccaatgcagttatttttggatgaacccttaacatgaattcagctttgtcttggtgggttagatcggcccagccgttcacgatgtccccaatggttaaactggtattatcgccagactggttggtttgtttaacgtattgctgaaagacacgctgaagaccctcggattttcgcctaaatctttttttctttactaatctatctaaatcgacttgtagcagggaactggttaaggcactagagtcaatgtgaacgtttttaattcgatcacaaaagatttgttgtagtatgcccgaaaaatattcattaccaacaggaccagaagagacacgataaagtaataacacggataatgcctttgcttttcttgataatgtcttcattgcttactccttccgtgctaaaataaaaatgggtacgtcaaggccgattgcgtcgtttttgattgccccaaggcggtagcttgtttcagcaggctaccgcactttttaccctgttatctactcatttgaattccgacctacgccagatggtttaaatcccttgcctttatcttcctctgcgataaatgggcagtcatcacatgtttcatgcatgtagcaggtcattgcatattctacccaagagttgaaactgtctccccaatcgtcttttagcgtaggttggaacttttcccacaattcgccgtcgagggcgaggtttattctatgtctcggctgttctttctgaggaatacacttctttttcatgtaataaactatacatcaacaatatatatatttcaagtttctttctatccgaggggatatttccgtgattatacagagcaattacccgggaaccgtaacaaataaaaaattgaatacttttcaaaaagggataataacgttcaatttagactgtcttcggtataccgaaatataatgtactattatatgggacaagcggcctcaaagtcaggcgactacaaaactgggaaaaattcgtaatacgataaaacatggcctgaagttgagctgaggtagcgtttgagattaccattggataactctggtcttggttgtggactgagggagttaggcaacgaccagcatgtcaattaatattttcaattgtaatatgaccttgagttttcgacttggctgctggctaactatttactaacccggagtcctgtctttgaaatcccttctttttagttttgtcgtaaacgaccgtatctgtcgctcgtctgtgttatactagggcagaaattgcaattccacctgcggacaaatgaaaaaggaggagcaagtgaaagataaagatagtctcgcccgggcagttcgtctcagccaaatacaacactttctctacagtaatccgtcaggtctcacgagcaaagaattagctgatctttgtggagtctgtatgcgaacgattcaaagagatattcaatcacttgatactgaccttaagatacctctgacgcagaacggagaccggtatgggattactgaaggctacattctgccaccggtttccctctcactttatgaggcaatggtaatatttctggcgtgccgtctagcgctccgtcagaccgatgagaacaacccgcatatcgaaaaagctcttacaaagatttcctccgtattgccttcaccccttgcgatacgtttgaaagagagcgccagttcggttggcgataaaaaggaagacttagagtttatacgtaactttgagaagattgcaatcgcatggacaacacaacgccaggtgagaatgaagtaccagtcttttgaaagcgaaggcatcaaggaatggtttctggaaccttatttcgttgatatgacgggtgttggcttctccgtctacgtcatcgggcatgctgtgagggaaggaaaggaaggcattattaccttcaagcttgaacgtatgaaagatttggaggtcttgaacaagcggttcgagattccccctgatataaatatcgggaagttgttagcatcaagctggggtattttcaccggagatgcaattagcattgttctgaaattttcaccacatgtgacacgccgcgtaaaggaatcagtgtggcatatttcacaattaattgaggaaactcccgatggcggatgccggctctctttacaggtaaacagtgcattggaaataaccccctggatacgtagttgggggccggatgtcgaggtaattgaaccgttgcgtcttaaagagcaattccgagactggagcgaaacactttacagattgtatcacgaataggtgttttgacaacgaaaatagaacaaatgtatgattagctctgccaagaaaagatacattgcaggtataacaacacgcgcggaggataacattgaaatacaacctgcttgaggagcactggattcctgttctcggaaccgatgggaagcttggctacgtgggtatcaaggatacgctgacccatgcagggcgcatccgttgtatctgcttagcaagcccactcgacatgttcgccgtgcaccggttcctgcttactttgctttattggaaagcagacttagcgggtggggtggaagaggttcgggcgtcgttgctgaagagcgaattgccgagcgcggtgctcggcggtatcaacaaggaagaccattgttttgacctcttcgataaagaatcgccattcctgcaggacacgtctgtatccatctccaaggaaaaagaaaagaagtcggtcggctctttgtttgctgaattagctgtggctactaacatagcccactttcatcacggcgacgatgagagtatgcggctatgcctgccgtgcgcaaccatcggcctgctccgtgtggttccgtggagccaatcaggcggccaaggaaagactccctcggtacacaatgcgccgccaattatggcaatggcaactggcgataacttggcggccacgcttggtttgaaccttgtaccgttggatactaaagcgggcaaagcgcagtggtccggctactttacgccgactgattcgacgagtccaatcccctatctcgaggcgctaacttggaatccccggcgcatccttctcccctcagcccaagaaggcacttgctggtactgcggtgaaagcaatgtacccacggtcgggaaaatcgtgtacatgaaaaacgaaaacacaaaaaagcgtgcagacgagaaattgttcgaatggcaggatccggctgccttttatgctctatacgatgtgagtaagaaatatgacaaagtcaagaacgtgccgtataaaacgataaaaagttcccgggagaacttggctaccgatggccgagacttgcagcgcctggcgcatgacgatccgtttcccgaatctaccgtagtcgtgaagaaccccaagcatgagggttggctcctggtcgttccatgcacaaatcccgcgaacaacaaaacctacgatcatcggcaggttgagctggcacaactttcaccagagacgattcgctccttaccgcctgccgtccccccgtccaggagacagggcatcgacggttggaaagaacccgaaagagtcgagcgcaaggacgttgagcattttgtgccggctgcggtgaggcttcttacgcataccgattgggttacgctatcgaacgctgcctacatggatatgcacgattcaccagcggcgttcgatgtttttagcggcttgtattggggacttcgtgacaagaagaccaaaggacttccgtcgcggaatgtggcgtggttgatgctcaaactgatggcatcagtccccgcacgtgcccgcactcttcatcccaacgccgaattctgcccgctgcagagactgccgaaacgacagagcaatgagcgcgacatggcgtccccctaccctgtttctttcccacgcggtcgtcggcttgaggccgcacttagcgccgcacttgttgacaatctgcgcaagagaaatcccgaacctgttgactggacacgtctatgttacggactagaccaattactcgactaattaagagaatcgaaaaaggagaaacattatgtttgtacagattcatatcctccagagcatgccaccaggaaacttaaaccgagatgacgaagggcagccgaaaaagtgtatttttggcggcgtcacgcgcggccgcatcagcagtcagtgcctcaagcgaaatatccgccattctatacaattcaaagaagtgttcggcgatgacctcgcaaccagaacgacatacctgccacgtatggtggccgacgaattgaggaagagcgacccaagcattaccgatgacgaattgaacaaagtcaaggccgcgcttgccagcaaattcaaggctgaaaagcgcggaggcaattcagacgaagcagcagagggtgaggaggcacaagcgcagcccgaaagctcttctgctgtgagcgatggagcagaccggacgggtcaactggtctttttcccaccaccctttgctaaagaaattgctgagcttgtcgtagaactgcgtgggcgcacccctccggcatacaaccggttcattgggaccaaagcaaagctctctaaagctgaagagaaggctctagacaaaacggttgatgagtttgtccagcgagcttccgcggcgagcaagaagctcacggttgacatcggccttttcggcagaatgactaccagtgacctggtcgtgaacgtggaagcagcctgtcaggtggcgcatgccattagcacgcatgagacgctaatcgagagcgactacttcaccgcgatggatgaaaagaagcaggaattcgcatcaactcagaccgagaaaacaggcgctgcctttctcggctctggcgataccgaaacgtttttcaattcagccgtctactacaagtatctcaatctcgacatcgatgccttgaagaagcacctttcctggcaaagcgataaggcagctcttgcagccggggtgcttgtccgcgccgcggcgctatccaatcccacaggcaaacagaacagctttgccgcgcacggcgttcctgaacttatcctcgtcgaagtatcggaagcaaagtgtccaatcagctatgcaaatgccttcctagaacccgttgacggtattaacctcatgcgtgaatcagcggcggcaatgagtaattacatcgacaccgtagtagcagccttctcgccgacagacatgagccgagcgctcctggcagtggggtctgcttcggtagatatcgcaaaagctaaaagagttaacacactcgaagaccttatcaaatccgtggttaaactcgtggagggtaccgcatgagcagtgaccacaatactttgtttctgcgcttggccggtcccatgcaggcgtggggcacctcatcccgttttcagttgcgccgcactgacctttacccgagcaagtccggcatactgggaatgttgctctgtgccatgggtgttaatcgtgaagattcacggcagccgctcaagctgctgacaccactactcatgggagtacgcgtcgaccggactggaaatcttgattgggattaccagacagcgggagccaagttaggcattcgaagcgccgacggcaaaatcaagataaccgcatcgaccggcgaatacgaaaccttgctttcgcgccgtcaatatctttttgatgcctcatttctggttgcgttacatggcgattcaaccataatcgcctcttgcgccaacgccctgagaaatccgacctggcctttgttccttggaagaaagtgctgcgtcccggccgagcctgtttacgccggcaccgaaagttttgacacgctgacccaagcgctttcgtcggtcccctggcgccccagaatcaacgccattgaccgcgagggtagcggggcgtctgtaacactcgatacatatttggagcagccgccagagagacttgtgcccatgggtgcccggctcgtttccgatgtgcccgtagccttcggtttctataattatgctccgcgtttggttatcaagaatcaggttacggtgccaatgggacaggcaacccaaacacagcgactgagcgcacgtcatggcaacccttacagtgaacacttctatacagtcgcgaggccgagtcgtctgaaacttgataacggcctttgcgtcttctgcaagtcccctgctgtggaagtgcatcatgtgtcctacgaaaatgccggacacgagaccgactctgatctccggtcgttatgcaagacctgtcacgatgcgtgtacgatgctggaatacggtcatggccgaaaagaaagagttgacccttgcaacccggctcaacgcacgaccattctcgaacaaatcaatcgtaatctcaaagagcaacatcctggccggcggcgcgaattgcttaaagctgtgcggtcgacctgagaaggtattttttgacatagcgcccggcgcaatgccggataaggagttagggatgtttctttctcttcttaaagttgacttggacattgcccccggacggctctggctccgtaacctttaccgcgttcatcagcgcctgtgcatggcgtttccatcctccgcgcgcaaagagggcgaccctccgttcttggaaccctttcgtcctgatgacttccagcatgttcacggcccgcgcacggaggaacaagcatttcttttccgcattgaccctctacccggcgggaatgctgcgattgtgatgcaatctggaatgaagccggactgggactatgcctttaacaacgctggctatcttcttgccgcgccaccccagttgaagacttatgaaccacaattccagaaccagcagcttctcaggttccgtctgctggcaaaccccacacggaagatagatacgaagacccgccaggatggcaccaaaagcaacggtaagcgagtccccgtggacaaggctaacctcgatgaatggctcatccgccatgccgaaaagtccgggttcaccgtcgagcagttgctcaacgtgcaaacgggctacgtcggcgcttccaatggcaaagatgacgaactcaagcggttcttctacgcccgctacgacggcatactccgtgtggcggatgctgagcatctgcacgattccgtcattcagggcattggctccgccaaaggcttcggcttcgggctgctttctctagcgcgtatcaaggagtagctatgcgtacattacatgaattgccgcgtttccgtgatagatggagccacctctacatggaaatggggcggctggatgttgatgcgaatgggttggtattccagcagggtgagagcttcgtgcgggtgccaattgaccagttaagcatcattatgctgggacccggctccacggttactcatgcggcggtgaaatccctttcccagaataactgcttattatcatggacagggcaggatggtattcggctctatgcggcaagcattggtggcacctacagcgcacggaggatgattcgccaggcgaaactggttagtgacgaggagaaacgtctggaagttgcctggcgtatgtaccgtttccgctttaaagaagcggttcctcaggtggttagtctggagcaaatccggggtatggaaggcatacgcgtgcgtcgcacctatgccgaggcttcagctaaatacggggtggaatggaaaggtcgccagtatgaccaggatgattggaacaaaggtgacccaatcaaccgcgctctatccgcggcgaacgcttgtctctatggagtttgtcacgcaggtattctttcagctggctattcatccgctctgggatttgttcacacgggtaaaatgctaagcttcgtttatgacatcggagacctctacaaaaccgaggttacaatccctactgctttcaggatagctgctcaaaatccaccggaattagaaagagtagtacgcatggagtgccgaaaggtgttccacgaattcagactgatggataggctcttgcctgacatcgcggaggtgctgggtgttagtgatgatactggagaaaacgccgacgagcttgaagggcgcattgtcacgctggctgttggaaccgaagacgggggtttttcttgggaacccgagcgcgagagttagagacgaactttgggcaacagcggtgaaaaaggcaaaggataccggcgtggtaatgcaaatctggacagaccagaacccgcaaggtttttcttaccggcaaactggtgaaagggatcggagatttattgacctggatggtctggcattggttgagttcaaacgcgctcaagagattgtcaaaagcaatttttctgacactagaacaccctagctaggcatgaggcataaaccggttagctttgggattgcagagtattccccacgtgtgtgggggtgaaccatagtcgtggcgcacattctggatggcatatcccgtattccccacgtgtgtgggggtgaaccgcatactctgcatgtctgacctgccctgggcaggtattccccacgtgtgtgggggtgaaccgaaacacactggccggacgaatacaatcagttaagtattccccacgtgtgtgggggtgaaccgcaacccatgcggtggctttggcttcctggtaaggtattccccacgtgtgtgggggtgaaccgcacgttgttttaacttctggtactttcaatatagtattccccacgtgtgtgggggtgaaccgcaataccgtccttctggaattggaaaggttcatgtattccccacgtgtgtgggggtgaaccgtcagcacagcgtgacggtgttccgcatggcctgtattccccacgtgtgtgggggtgaaccggactcttttgccaacgtgtgaacccctagtgcgtattccccacgtgtgtgggggtgaaccgggatgggagtggaccaaagcgatcgtgtgaccgtattccccacgtgtgtgggggtgaaccgtgcggtacacacagcgccaagttggatgttgccgtattccccacgtgtgtgggggtgaaccggacgatggccagcccaattactcaaaggctgtgtattccccacgtgtgtgggggtgaaccgcgtgctgaccgtatttattgctctaaggagtggtattccccacgtgtgtgggggtgaaccaggattctctaacgcccaaaaggcagggcttgatgtattccccacgtgtgtgggggtgaaccgtaccaacgagggcgccggcgggaacgagtgtagtattccccacgtgtgtgggggtgaaccgggatactgcgggaagctctgcctttggaagggagtattccccacgtgtgtgggggtgaaccgctcttgagttgggtaaagctccaggagtggaggtattccccacgtgtgtgggggtgaaccgtgcaggcggccgctaaaggcgtcaaggctgaagtattccccacgtgtgtgggggtgaaccgagatggtaatcggcgcagtcgtaacattaagtgtattccccacgtgtgtgggggtgaaccccattccggccggtggttccggcacaccaggtcgtattccccacgtgtgtgggggtgaaccggactatgagtttggagatgttaagaaaattaagtattccccacgtgtgtgggggtgaaccaggtaagtggtcggccggactctggcacgagttcgtattccccacgtgtgtgggggtgaaccgctctgcgagcggttcgagacctgataggcgatgtattccccacgtgtgtgggggtgaaccgcaccttatagtgggggattcgccagtgaaaacgtattccccacgtgtgtgggggtgaaccgtcccccgacagggtatatttttacccgactacgtattccccacgtgtgtgggggtgaaccgcactggactgggacgggctataccagcgcacacgtattccccacgtgtgtgggggtgaaccagttcaagcatcgtgttgggacaaaaggcggtagtattccccacgtgtgtgggggtgaaccggtcacggtgtacgaaagtatctgtatccagttgtattccccacgtgtgtgggggtgaaccgacctcataacaagccacgatgcttttacattcgccgtaaatgcgaaagtattccccacgtgtgtgggggtgaaccgggaactgcctgcttcgtcaagcctcatcaaccgtattccccacgtgtgtgggggtgaaccgcctacatagcgggatttattgacggggaaggtgtattccccacgtgtgtgggggtgaaccggactgttcatcctccagttgggataatccagtgtattccccacgtgtgtgggggtgaaccgcagtatcaggcagatggtaatcaggcgtttgcgtattccccacgtgtgtgggggtgaac

SEQ ID NO: 7 - DNA, 32759 bp, unidentified
  misc_feature 1..32759: note = Gaa0073932_1000118_organized_organized
  source 1..32759: mol_type = other DNA
acacgatccgcttgacctcgttttcagcgtcttcgattcgcttggatatttccacctcctgctcccgcgtcagcagcggcacctggcccatctgcttgaggtacatccgcaccgggtcatccaaaatgtccagccggtttttatcgtcgtcttcctccggctccggctgctttacccggtccacctccgcctgatctacaatctccacctcaagattccgaagcttggtgtagatgtcatccagctcctccggtgtcaccaccgaatcaggcagagcgtcattgatatcattataagtcaggtatccctgctcctgcgccaatcgcaccaattccttgaccttttccgccaggtcaatcgtctgcggttgcttgttgggatcctggttttcatccaccgcaacaggcgcagcttcctccatcgccgtcttcgcaatggattttgccgttttcacctcgcttttagccgcggccttggcaacagcctcgccgcctttcttcgtttgcaactgctctgcctttttgctctcagccgtttctttaaccttcaaagagccgggctttttatccttgttttccacttcggcaggcttgaaatccttcttcttcggtgggacgccatctttcggcttggcctgggcagaagcagacgctgccggtttggagcccttggcctgggaaggttgggcgtgggccttcacttccttattggacgataactttcccttgccagccttcgcggctggctttttcacctgaggttttgccatagatgcgttttagaactatcggctacaaattctcgttataataaatagccttatactttaacgcttttctgttgtgtagtcaatctttgcttcaaaatcaaactttgcccacccttgctgccctgttgaaggcgtaaggtaccacgtaaaaaacactacatttccacaaaacacgggcctgtcttccccccctcccctcgatatttccacccggttataatccatttgtaaccttttccgccaaaaatgctcttggtatagtgtttggaacgtaggcggccaaccaactgcatccgccgcctaaacgccactcatatctgtgggcatgaccgaaacggtccataccgaagtgttgccgctccattcgacggcggatttcaccgccgcagtggagaaagccgccgctttgcttcaggctggcgaagttgtagcggtccccaccgagacggtgtacggcctggcagctaatgccctcaacccacaagccgtagccgccatctacacagtcaagggccggccttcccgcaatcccatcatcgttcacgtggccgatgaaagcatggcccgccaatgtgtcgcctcctggcctcccgctgctgccgccttgtcccgtgccttctggcccggacctctgaccatcgtcctgcccaaggcagccatcatccccgacatagtaactgccgggggccccaccgttggcgtccgctggccgcaccatcctttcatgcaagctttgattcaacgttgcgcctttccactcgctgcccccagcgccaacctttccaatcgcgtttctcccaccaccgccacccatgtgcgccaacacttggaaggacgcatcccccttatcgtggacggcggcccctgccccgtcggaattgaatccaccgtggtggcccttaaaggtgatgtttggcgcatcctccggcccggcatcattcacaaggacgctctggccgccgtcttgggcaccggctctgctcatgaggccgcctcacacgatagcaaccgccctgctttggaaagccccggccttctgccgcgccactacgcccctcgcgcacaagtgttggtttgggcctgggaaaacgaagactccctgcttgcgcagattcgccgcatgggggcaaaaccggaggaaacttacgtcttggcgcactcctgtgcgccccgccaaaaggactggcttgaagtcagcgttgcaccgcgtgacccggaagcctatgcacgcgccctttacgcggaattgcaccgggcggatgctcacaatgcacggtggctggtggtggaacagctccccaccgatggcccatggcaggccatccatgaccgcctccgccgcgccgctcaacatgaagaaacttgaattttacgtcggttgttgattgccatgcctcaaccgcgttacgatgaattgacgttttgatgaaaaacttgcaagctcgaccaatgacacgactgggaatcatcgcaagcctggtggccgtcctcggcgccacggcctggggtcagaccaaccatctgacggaacgcgccatttctttggatgaatgtctggaaatggcattggaaaacaatttcacccttcgcattgagcgccacaacatcgagcaagcccgcttcgacctcaggcgctctcaaacagcctacgacccgcagcttttctttggcctcacccgggccagaaacaccggcctcaaccaaaacttcgatttattgggcaatcgctccgagaactcctcccaaaacacggtggacaccctcgaatacggagtcagcgggctggggcccatggggtttgaatactcggtgccagtgagattcacccgcagcttcggcgacggctacacgtacaacaccctcgatctcaacaccttcaccttcgtgccggtgggttacgacgagtacgctgcccgcttcaacatcgaaatgcgtcagccgttgctccgcaacttcgctgtggacaacgtccgccgcaccatccaggtcaaccgcaaactgctcaaaatgtccgagcaccagtacgaagcccgcctgatggaaatcatcaccttggtggaactggcatactatgaactgatcttcgcccgcgaaaacgtcaaagtccaggagaaggccctggaactggccgaaaaactcctcgataacaaccgcaagagtttcaatgtcggaagcatggccattctggaagtgcaccgcgccgaatctttggtcgccagcacccgcgccgacctcctggccgcccagcaattggtcaaagtgcaggaaaacgtgctgaaaaacctcatcaccaaagacctggccgagtggaaacaagtttccttgagacccaccgaatccctgatggccattccgcaagccctcgacgtgcgcgaaagctggcgcaccggtttgaccaaacgccccgacctcgcccagttgcgggtggacttggaacgccaggacattgacatccgttacttcaaaaaccagaccctccccaacctgaacctcgtcggttcttacggacgggatgccctcggaacgggctttgaaaacacttggtcagacctccatttccgcgacaaagccgtggactactattacggcgttgaactcagcttcccatggggccgtcgcgaggccaaataccgtctggcgcaggaaaaagaccagcgcctgcaaaaggaattagtcctccaaaaactccagcaggacatcctggtccaaattgacgatgccatcgaagccgttaaaaccagttatgaccgcatcgccgccacacgccaagccgctgattttgcccaaaaagtcctccaatccgaggaatccctcatgcgcgtcggcatgagcacctccttccaaatcctgcaataccagcgcgacctcaccgcagcccgcacccaggaagcccgcgccatcgcggattacaacaaggcattggctcgcctagcccaggcggaaggctccaccttggaacggcgcaaaatcaaattacaattcaaataaaaccgccccggaaacgcgaaggttaaaagcctctcttgcagtggctgctgctacctgctgccaattcgaaaccaaaagcactgcgggaaaacttcccgcaattacaccagcccctccaaaatcatcggcaaccgctccaagctgcggttaagccctgcccgcgcaatggccttggcactgtcggtttggttggggaacaacaaatgccatgccggcgtttcaaaccacgcttccgccgccacgctctggtaattcccgctggtcccgatttgggcatgccgcaacagcagatccgccacatgcaccgccgccacaatttgaatatgctggctggcgcgctcgggcgcatggtgaaaccgggccgactccaccatcacctgcggcagtcggtgttgctccaaatacaacccgcccaactccgcgtgatccatccccagcacctcaatttcaatcgcctgcaacggcgcttttgtctcggatgaacgccggtgaatctcggcaaaatgctgcggaaacaccgcggccatcacaattttgcccacatcgtgcaccaatcccgccacgtaatccgcctcgtcattgggcattcgcaccgccgacaacacttcccgcgccataatcgccgtgccaatgcaatgacgccaaaactcccgccatggaaattccgtgttgcccgccatcttctgaaaatcctcgatcaccggcgtcaccatcgccagttgccgtatttgcctcagccccagataaaaaaccgcctcctcaatgctgttgaccggcacattcagcccaaaataaaccgagttcaccaaccgcaacaaccgcgccgtcaaactagggtcccgccgaatcacctcgctgatttgcgccgtataactctgatcagccgccaacaactcccgcagcgcactgttgacagtgctcagcgatggcagacgcgggcaggaatgtaaccgcgccgtgatctgttgccggttcagcggcgtcagccggttcgtagtggccacttgactcatactcatcttccctatcggcagtcagaggaaccaactttaataaataataaaaaaagacctctggttaccctcccagcgtccgatttttagacctccacaatcaaacagccttgggtagatacacagaagccgcgcttgacggcctgccctgctttggttaccaaagcatcaccaatacagcacaatacattcgcgggccgggccggtttggcgtccctccccgccttggaaaatcacatgaatactgagaaaaaaggcctgtttgtcaccgccgacggcagaaacgtcgccttcacctttgcgctggtcagctcgttgtttctcctctggggattctgcaacgggatgattgacgtgatggacaaacatttccaaaacgagctcaaactcaccctcagccaatccgcctgggtccagtttgcccattacctcggctatttcatcatggccctgccggccggctggctggccatccggctcggttacaaaggcggcattatcgcaggcctcttgatggtggccgtagggggattgtggttcatcccggccacacacatcgccaagttctgggcctttttacttggcgtctgcaccatcgccgccggtctgaccttcctcgaaacggtggccaacccatataccaccgtcctcggcgacaaacgcttcgcagccactcgcatcaacctggctcagtcctgcaacggcgtcggctggatccttggccccatcgccggcggcctcttcttttacggtaccgatgaaacaggacgcagcaccggtgccgaaaccttgtggatcccttatgccggtgtggccgtggtggtcatcctcctggcggtgattttctttttcgcccccatgcccgacatcaataccgaggacgactatcacctcgacgataaaaacaatgacaaaaccaccgccattaaaccggcttcccgtgggctgacctacgggttgttactcgtgaatatcgccatcctgcttttggtgtttggaatgataacatggctcattctcagcgcgaccaaagttggcgaatccctcaaaggcctggccgctgccctgccgcatccttcctccttcaccatcaacgcagaccacgccttgaccgtcgtcctgttttgggcgggcgtcgccatcatgatcgtagcggccattgtcatggcccccatgaccaaaaaaatctcccaccacagcatctggtcccacccccatttctccggggccgtggcggcgcagtttctatacgtggccgcccaggcgggcatcttcagcttccttatcaattacgtgacctcagaagtcccggacctccccgcctcgtggcaccgcgacagcaccaaaaaatggattgaaatcaacacctccttcacacgcgatgacctcaaggatttgcctgcgctggcggctcgattgaaaaaccccaccgaccccgtgacagccatggtggccggcaaattgtcctccgacactcaggcagccctcgcagagtacgtccccgacaaaaccaaccccaaattgctcagtgtgcccttggtgcaggatttgaaaagcattgtccgcggcaccaacatctacaatccccagctcttcgctggtgtcaacctcagccccgccacccgcgaaatcctggccctccctgaaaaagaacgaaacacacccctcctcaaccgcctgctccttacagacgcctttgccggtttacttaattataccccaggccgttattgcatcagtgacgccggcgcttccaacctcgcctctttcggattcttctgcttcttggtgggccgcttcaccggagcctggctgctgaaacgtttctccgcccaccgcatgttgggcctctacggcctcatgaacgtgctggtctgcctgctggtgttcttcaagctcggctggctgtccgtggtgtgcgtcttcctcagctatttcttcatgtccatcatgttccccaccatctttgcactggggattttcggcttgggcgtccgtgccaagcgcgcctcggcttacatcgtcatggccatcatgggcggtgccatcctgcccaaactcatggggtacgttgctgaccaaaccaacatgtcccgcggtttcatcgtgccgatgatttgcttcgtggccattgccctttacggcttcggttggagccgcctcagccaggccgaatccttaagcggcctgaaaaccaccgccggccactaaaaacaaggctcccattcttgcaccacccccctgcgctggggccggggctcgtcttgtcttcaaccaatccgcgctcacactttctccggcaccacaaacggcggacggtactcgcgccgaagcagccgatccgcctccggattgttcacaaacttttccttgtcggcatcaaacatcaacttgggccccacccacaacgtcgccttctgcgggtccacaccaatcgccttggtgttttccaaagtcgtcatgatgctctcgcccaccacctcgttatcgctgaaatccttcggcttctcaaacggtcgctccttggccaaccggtaggatatattgcccaaatgacagcacgcgctcgaaaggtgcccctccaaaatgtctgcgtgcagattttcccgcttgcggctgcgcacgcactcgatgaaattggcgaaaatattacccgagcgcgacggcgcatccaccttggccaacggctccccttcggttttgtctttggggaaaaacttcccatccttcaccacaccggcctcaaagaaatattcattggcaactttgttgggatactccttgagcttgcccacaagcccccgcgtctcaaacaacagcaatgtcccaccaaaatcgagcaccgtcaccatctgattcggcgtctgcccttggtccttgtgatccggcccctcaacgtaccgcccgcccaaactgatgaccgatcgcggcaacgtcccaccaatcacccaccgcgccacatccatctcatgcacaccctggttgccaatgtccccgttgccgaagtcccaaaaccaatgccagttgtaatgcaccaaattccggtgaaacggctgtttgggcgcaggccccagccagatatcaaaatccaaattggctggcggttcctccacaggcttgaaaccaatgctccatcgtggcttgcaacaatatcctttggccaccaacaacttcccgtaaacaccgcttttggccaacgccaccatccgcacgtccccaccggcgcgccgctgcgtcccgtgctgcacaatccgattgtacttccgggccgcctccacacatttgcgcccctcaaacacattgtgactgcacggcttctccacatacacatccttgcccgcctgacaggcccaaatcgtggccagcgaatgccaatgattcgtcgtggcaatggagactacgtcgatttccttgttctccaacatcttgcggatgtcctgataagtctgcaccccctgcaacttgctcgcacgtccctccaacaccttcaaatccacatcgcacaacgccgtcactctgactcccggcaatttctgaaactcgctgatatgattggccccacgcccgtttattcccaccaccgccacacgtatggcctcattggcacccacggattgcgcatacacacgcggcgccagccagatgccggcggcccccaaagttgcggttttcaaaaaagcacgacgcttcaacgacgaagactgcatgtaggctttcatgcccccactctaatctctccgcgccccgcttcaatcccaaactccaactcccaccaaacaccgactaattatagatgaatggcagtgcaggtcttcccaacataagatgagcttgcgcttgcccttccacgccgcggccttacattgagcgtgtgcatgatgcttcggatgattggcgccggcgttttgaacgcctgcaacagttataccacgtcagccaggtgctccattccactctcgaacctcagcgagccctggaactgattgtggaacaagccgtgcagctcaccggcgccaccagtggttcgctggttctcctgaatcctcataccggttttttggaaattgaagcctctcacggccttccacccgaggctcaaaaactcaagctccgcgccggcgaaggcatcaccggctgggtcgtccgcaccggccagccggcactcgtcggcgatgtccgcaaagacccccgctacatcatggcacggcccggcgtccgctccgaattggccgtccccctccaagtcggcgaagaaattcgcggcctcttgaatgtggactccgacaaacttaacgccttcaccgccgaagaccaatccctgctggaagaactctccgctcaagccgctctggtcatccgccaaacctggcaatacgaacaacaccggttgaaagcccggctgtttgaaagcctcgtcgccgtcagccaaatcatcaattccaccgtcaatctggacgaagccctcgccgccatcacccaacaggcctgccaactcatgcaggcacgcctcagctccttgctcctgctcgatgactccggacgctggctggacctccgcgctgcgcacggcgccggccccgactatatcaacaaaccgcgcctggcagtcgaggacagcctggtgggctccgttgtgcgccgccgcaaagtgcttcaggttgagaacgtccaaatctcccagcgctatcaacacgtcgaagtcgccgcccgcgagggtctggtctcgctgctcagcgcacccctcttgtttgaccaacgttgcatcggcgtcctcaacgtctacaccggccggccccacgtcttttccaacgaggaaatccgcatcctcaacgccctggcgggtttatcggccgtcgccatcgaaaaagcccgcctctacgaacgggtggtggacctcgaagaagccctccgccaagctgaaaaattcagcgcactcggcctcttggccggcgaagtcgcccacgaaatccgcaaccctctcaccgtcatgaaaatgctgtttcattccctgaacctgcactttcctctgatagacccgcgcaataccgacgtgcgcatcatcgccgacaaaatggaacaattggaccgcaccgtcgaacgcgtcctcagcttcgcccgcacagccgagccccagtttggccccgtgcacctccaacacgtgctcggcgagctggaactgctggtccgccacaaattgcgccagcaaaacatcctttggaaactcagcctcgaacccgccctccccgccgtttggggcgatgccacccaactggggcaagctctcctgaatcttgtgctcaatgccgtccaagccatgccccaaggcggagaattacgcatctccgcccgccgcctgccgcgcgaccccacccgccccgccacccatgtagtcttggaagtagccgacacaggcgaaggcatgagccccgaacaatgccgccgcgcctttacccccctcctcagcaccacccgcaccaagggcaccggccttggactggtcttggtgggccgcatcattgaagctcaccacggacgcatccaagtcacctcccgccccaaggcaggcacacgcttccgcatcacgctccccgttgaaaaccaaaatcctgcgtaattccccccaatcgcctccggcggcccccttgacatcaagttgaacgtgggacaagttgccattgttgtattatgaattttgcgtggcttagcctggaccttgttttgattgtgctgtttgccttttcgctaccggcacttttcctatatgtcatccgccggtggctccaaaacagcgaatcggcaaacccatcggtggacaaccaaacaccggacccaggcaaccattcatctcactgaatgaccaacggattttttctcgccttcttcgcggcttcctcaaaacaggctttctaatatgcttaagccctaaggaggctcaagccatagggttgtatgccgctcactctttgctcgggaaaaaacagctccaccgattatttcaccattagatctcccttgggcactgaaaaactttttcgctctatttttgctgtaaccgccttttttgatttgttctgaccccgcggctgggcttatcttgaagcagtatagcgatggaccctgcccactcaacgtccagccgcaaccatcgggccacgccacacgcgtggaggaggtgttcatgttgacctttgaccccacgcgattgcaagcggccttgcggttggctgcttccgagctggtcaaagctcgtccggcaggttctccctggcagggtgaaatttccagcagccccacagccaccgctgccgccatctgcgccctcagcctggcggataccgagtggaaaagcgccgaggacaccctgcgccggctcgcgttacatggggcggagtggttgagccaccacgccaacggcgacggcggatggggcgacaccccgcgcagcttcagcaacctcggcgcaactgccctcgcttgggcggccttcgggctggcaccgggaggcgaagcagcctttcaggaacccgcccgtgccgcagagcgctggatatgccaaaaagcgggcaataccgcccccgccatcttggcccccatgattcgcacccgcttccgcggggatttggcgctggccggtggcgttttgatgtgctgcgcactggcgggacgactcggcagtgacattcgcgcctggcgccacgtcccctccctgccttttgagcgtggagcactccctcccgaatggtgggcgtcagtccaaccgcccgctgccagttgggccatgcccctcctcctcgccacaggccaggctcgccatcacaatctcgcaggcggctgtttcgtggtccgactgctgcgcgaattctttcgcaataccactctgcgccgcctcacggccttgcaagcagaggacgggaattttcaaacctcccccctcttaactgccctgaccaccatcggcttgctcgccgccggctacggtcgccatccagccacagaaaaaggcatccgcttcctggtaacctccgcacgtcccgacggcagttggccgctgattacaagccaggacgtccgccttaccgcgcaagccacgcaagcctgcaccaaccccgtccccgggcagccgtggctgccgggcctcgacgaacttcatgccttgcgccattggcttctcgcccatccgcaacacgatgacccggagggactccgcctcccagccatcggttggtcggccagctcacaccccggcgcgtggcccgacaccgtttccaccgcagaagccctcttggccttgcgctcgctcggtgacccgggccctgaggtcattcaagccgtcctcggcggcgtcaattggttgctgttgacccaaaaccgcgatggcgggctgcccccttatcgtcgcggctggtgccacccggaacaagaaggcagttgcgccgaggaaaccgcgctgaccttgcgggcttggctggcctggttgcacgtccttcccccggaccaacaacaccgtgttcaaacctccatccaccacgccatccgttttctcatggacgagcaacgtcaagatggcgcctggcgttcacactggtttggcaatcaatacgcgcatcaggaggaaaacctcaccatgggcaccgccggagtcctcatggccctgcccgaactggtggcccgctcctatttcgagttggtcgaaccactcaacagcgccgtcctctggctcgtcaaaaaccaaaacagcgacggcagttggggcggtggccacggcggccctccctcagtcgaagaaactgctctggcactcgaagccctggccgaaacactcctccaacctggccccctcttgctggaaactcgtgaacaagcacgcaacgccgccgcctggggcgccaactggctagtccaacatatcgaagccggcaattggaccacacctgcccctctgggcttgctctggggacaattctggttctacgaaaacctctggccggccctcggcgtcacccgcgccctggcccgtttgacacgcctggtttaatcccgccaccccccggcgttgtcatccgcggcaggcggctcatgtgccgaaaaaccatccgcgacaatggcttcttcggcgaataatgcaccatcatgtcctgcatccgcatcaacacccacggctcccgcggcgcataaccgtttttggtgaacacaatgtcatcagccacgtacacacacatgtgcacagtccggacgtcctgccccatcggctcatacaacaggatgagatccccaaacaaatccgccttgggcacaatccgatactcgctccgcaacacctgctcctgaaatcgtgcatcaatgaaccggtcctccaccttgtcccggtgaaaattatacgacgaccaaaaacaatccggccacctccccgcaataatatttgtgggatgcggatacgaataaagcaacagccgcggcaccggcggaaaaaaccatgccacattcacactccccccctgcggcaggcgcttcaatgactccaacaacggccgcacctgaagcattcggttgggcagcccccaatagcccatcagccgctccacatcgctggtgggagtcacccgcaaattcaccataagcgtcggcactcgtgccaccgtccggctggcttgcagataatcctctctcgttaacagttttggcaggacatgccggtccgcaaaacacagcaaaccatctttccggtacatcatccgctccatgagcttcagcgcatcttcagacaaactgcattgcgaaaaccattcggaaaactcagcattaaccgcaaacggataacaataagccacattttgttcatcctcggccaaaatggagtagatcttggcccgcggctccggcggcatgtcccgcagcacttcaactgtcggctccacataaactcctcctgtcgtcgcctgccaacggttcgtgtccaacaagctcgcctccaacaaactcggcaacccgcaactttttatgaaatccctcaaagtctccggcttataaccggcaaaaaaccacctgttcgaggaagggggaggcggatcgtacgcaaaatattccactggccgctccaaccaaattgggatgatttccaatattccccagcggtttgttgaggcttcatccttcgccaccaaaccgttgttggatgagcttcgatcagagcgcgaccataaagtgcgaccaataaattgcgcacccaacgtaatcaatacccctgcaagaaccagccataaacttgggagaaattgccggcgaggcacggccccattctgccgctcgctccgtgatgaagtcttggctgggcgcattttcataaacagcctcccttaccactcaaacagcgcccaaagaccagccggcccaaaaacacccctctcccgtcgaagtgtttgcctgttattcgccggatgtattaatcgcccgcccttaaggacgttattcccttgcagccatcccattcaaataaagagacgggacattacctgcgaaaggttacaaggacaacggttttgtgaaaaaaattagcctgaaaaccctcacattgtgaacttacctaccagacggtaagttccgccagggacgcaaagggttgacggtcaaatccgctcacagcctccagcttcaaataacggacctgtaccggtgccggaaacgtcaattcctttggattggcatcggcggacaatgtgccccgcatcaaaggtgttccccagttttgtccgtcgctgctcgtgtaaatggcaaactcgcgtatccacccatttttattgttgtcctgcctcggcgtcagagtcacaccacgcagagtggccgattgttgaaactccaaaaccagccaatgcggaaaagccggagcttcctccccccatggcgtgtgccaaagcgtttgcacgtccccatccacgaccaacttgcccggatgctgcggatgttcactgctcacccgcgatacccggacccccagtttttccatcaaggaaggcggcctcaacaacttttgcacatccgccggcgtccaccgatgcgcgggtttgaaacgtggaccgctcatgtaattcaaaagactgcgccgaagctgccttgccaccggacgctgctccaagtctgtattcaaatccatcgcacacgccaacaaacgcccccgtcccacttgcgcctcgatgattaaccccagtttgcgcgacgtaaaccaatcgtctatcgcatacaccaacggccggaatgtcgcctccgtgcccgtcaacacaaacggatgcgcccgcgacaccaactcccaccattgccagttggaatgatattcagtcggaaaatccgccagcgccggatgcctcggatcgcaataaatgcccaaagtatgcggcgcctgacgccgcgtccacgctgtgttccaaaagatgctcgaaaaaccaatttgcacccgccccaacgcatccccgcgaatggccgatggcggcggattcaacaacaccttgcctccgcgctccaaaaccgccaaggccgccgaatccaaatcagtcgcccaatgaatatcctgcggcacactcacatccaccttggccggatacacccacacatcccaatcattcgcataaggcgtgccttccaccgccagctccaaattcaacttcgcaggcgcaggcatccccttcaatggcacctcaatctgccccaccggactgcagattccagtcggcaacggaccagtgaacatcgtccccgtcgccaactcacgcttcccctgccgcaaccgccagcgcaccacaccctgcggcaaatcctgcggtgcaaaccatcccaactccaccccggcctgcagcgtgtcctccgtggtgaaaatccgtctcggcaatcgcgcaatcggcaccaccggcccggcaaaccggtgatactccgcagccgtgatatagcccttgccatcccaaaacggatccaacacccccaccaacgccgtcccctgcccaggaaaatcatgcaaatccaacaactcgaatccccccatccccggcgtgcgtatcgccgactcaatatcctccttgtaacaaagcgcctgcaatttccccgacgccagcaaaaacgcccgcgccataggcgccaaaccgttctgcttcaaaaagtcctgaaaaatctcaaaattcttcggtttcagccagcccttgtatttcggcatctccgcaaagttgggaaacacacaccattgcccaatctcatgactcaccaccggcgccgcagcccgctggataaacgcccggtaatctgtcaccgtctcaggcggacgcgcattgatgcgtgaattcaatcccccgccccatgcctgaatgcgcggctccggtgtgatgtgaaaatcattttccggaatcatcggccaacccgccccgctggtgtaaagccgccgcgtatccaggcccttgcaataattgatgaaatcccccagccaccgattctgattctttcccgccggttcgttgccggctgaaaacatgataaacgacggatgattgcccaacacccgaaagatgcgcgctgcctcctcgtgcaaccacgcatccaccttgcgcccatcccccacactgctccccgaattcgcccaaacaccggcctccacatacaacaacatccccgtctcatccgccgcctgaaacgccgcctccgggggagtatgcgaatgaaatcgcaggtgattcaacccatgcgcctggcaaatccgcataatgcgcttccacgccgccacatccgtcggaggatagcccgtcaaagggaaaatgttgcactcaagagtgccacgcagaaaaagtttgcggccgttcaaagcaatttgcgtcccctgccgcttcacctcccgcaatccaaacgacacctcccgttgatgacgcaatacccgtccttcatccctcccctccaacgcagccgtcaattcatacaaaaccggcgaaaattcatcccacaacaacgcatcctcttccatctcatactcacactccaccaccccaccctccggagtccacgaaaccggcacagtcaccggcggcacccgatgctgccgtggtccattgtaacttcgcgccgtcaacgtcaacgtcccactgccggagcgccctgtgagattgcccaaccgcgccttgataaccgcacggcggcgagccacgtccggatacacctgcaaatcgtccaaccacacagggtccgtggccgttaattccaacacccccacaatcccattccagttgccttgcgtgtgatccgaaacactgtgcgcattgatccccacctccacatgaagccggttgtccacacgtaccgtcagccgatgccgcccaggcgtcaacgccgcagtacaatcatattcatgcggcgtccccaaactgttccgcgacccaatccggcgctcatccacccacacagtagtctcccaatgcggccgctccaaccgcaacaccacccttttgccctgccacgacgcgggaatctccacctcccgctgataccacgccacccccacataatgccgctccggttgcaaccaaaacggcaccttgatattgcccggttgccggtagggcgcataacgctcatccttgaaaaaggatcggtctatgatttggcctgtccactgcgtctccatggtcaccgggtccccaaatccctgctcctgcaatgaaccaggcaaccgcagccgctcctttaacacccggccatgccacccctcccccacaccggcatcctgccggtccagctcaaaccgccatgtccccgccaaattgatgcgctccgcagctcctgcgtaaaagttggccaatgcaaaaacaatgcatccaaaaaatccaactgccagcctttgggcgcccttgagattgaggtgttgggtcatcatgttgcaagggcagtattgcaaatgccgcactccagtttcaacacccaacaatcacccctctccaacggtggacaaaaccaaacaactcggaaatacttccaccatgaaacatacccttgcctgcatcgcactcctggcttggctggccacggccctgcccgccgccgaacgtttccccaacctcgcagccggtagcaacctcccccaaggccccggctttgcaagcaagttcaaagcagactccggcatcgccaaccatccctccgtcatcttttccgatgattttgaacagggcgatttaggggcacgctgggacgaaaaaggcgccggcaaaggcaaggccctaagcctcgtcccaccaaacgatacacgttgtggccgccgctgcatgaaagtcgaagcccgactcggcgaaaaccagggcgacggcctcaccaaatggttcaaccccgtggacaccgtgtacatccgcttctacgtcaaatttgaccccaactgcgactatgttcaccacttcgtcacacttcgcgccaaccgcggcctccgcggcgcagaccgctggagcggctttggcggcgcaggactcaaaccacgcggtgacgagcgcttttccacagccttggaaccttggggcaattgggccaagtggccgcccccgggcaaatggaacttctacagctattggcacgaaatggatgtctcccgtgatggcaaatattggggcaactccttccaagcagaaccccaggatgtcattcccaaagaccgctggatttgcgccgagttcatgctcaaacacaacacacccggcaaacccgacggcgaacaagccttttggattgatggacgcctcctcggacattggaaaggcatcaactggcgcaccaacccctccctccaagccaatgccctgaccttggaaagttacgtcaccgaccgctggacaaaaaaccccaccaacaccgtctatttcgacaacctcgtcatcgcccgcgaatacatcgggccggccaccgccccctaaatgggcagcgtgggctgcggatttgccgtccctttgtccggcactgcggctgccgcagcttgcgcagcgccattctcaacctgcaccaacgccggccgcaacaccttgccctgatacgtatatcccggcgccagcgtcaaaaggatgcgcgtgcccgcagccggtggctccgcacactgatgggcctgatgcatctgcggatcaaacccctcgcccgcctccggagaaaacgcttgcagccccacccgccgcgccaaatcacgacaaacattttggaacaacccgatttgctccaccaaatgcggctgcccgctctgccgcgccgccatgtacaacgcaaacacctggtccagcagacgcaacgtcaactgcagccaatcctgctcctgccgccgcaacttgtccgcttccaaccgcaacgtcgccttctcggtatcattggcctgccgcagaaaatccgtgaatgccttggcctcagccgccatcccgtcggccacttccttcgccgtttgcaccgtccgcgcacattcgccctggatgttcatccacgcgcccgtagcctcggctatctgctgcttgatggcctccaaattttgaatctgcgccactgtcgaacgcaacgcatccgcctccgccagcttcatctcagcccggtgctggagaataaacggcgtcaccaacaaccacgccccggcagccaccgccgccgtgcacagcgccatattcgccgtgctcaacggccactcgctttggtacacaatgccaccggccaccgctaataaaagcaggtcgcccaataaaaacggcaacaccgacggtctgggagtcgcaacgtcactcatgcgcccgccagcttatgtccttaggcacaacccatgcaacagcgttttaccccacccacaacatcgccactccttttgtccaaatcaaagaacccccaccccaaagcaccccgcccctctcacaccccctgccccactccttttccccgcaaaatatggccattccttgccaaagccggtgtagcaaacaaccgccggtattcataagtttttcctgccatgctgagacaccagcctgtgcgtgttttatgaaaacagtccccttccccagaggaaggggcgaataaccggcataccgccacaaccaacgcacaggcccggaccccggctggccatgcaaatgagcggaagtcattccatgggcgcgcccacggccgcagccaacgtccccgaccttcaggggaacgggatcgaaggcttggcaagccccttgccggccatgtccagcgcctacgacttcctccaaaaccaatacgtttacgtcatcgtctccgcccgcgcccgcggattatccgttggcgtcaacctgaaccccgaacgccgctgcaacttcgattgcctctactgcgaagtcccacgccccatggccggtggaaacccgccggtcaacgtcgaagtcatggcccgcgaagtcgagcgcaccctcgcctacgtccaatccaaccgcctgcagcagcaccccgtcctcggcacgctcccacgcgaatttgtccaattccgccaccttgccctcagcgggttgggagaacccaccctgtgccccaactttgtcgaagtcgtccaaacactcgtccacctccgcgctttagggcggctgccctacttccacctcgtcctcgtcaccaacggcagccagctcgaccaccccagcgtccaggctggccttaaatactttaccgccaccgacgaaatctggatcaaactcgacgccggcacacccgagtacttcctcaaaatcaaccgcacacccgtcccctacgaaaaaatcctcaacaacatactcctcgtaggccgacagcggcccgtcgtcattcaaagcctcttcgtctccgtggaaggcgaggaaccgcccgcccgccaaattgagcagtacgccatgcgcctcaaacaactcaaacaagacggcgcccaaatttccctcgtccaagtctattccgccacacgccccacccaccaacccgcctgcagccacctccccttgaaatccctcagccgcatcgcccaaatcgtccgctgcaccaccgaactccccgtagaagtcttctaacccccccgacctccgtcccacttgacagcccgtggcaaactcctacgttgccaaccatgcatcgcccttccgcttcatcctttggcatcgggcttattctggggttgctcctgaatgccccggcacccaccttcgccgcgttggatactcgcgagctgcacacagaagtcaaaatcaccattcaactgaaatacctcatctggctgccggaaggttatgccttgccccaaaaccgctctactcaatacccctgcatccttttccttcatggtgcagaaaaacgcggcacaaacataaacgctttgcaggaccttggaccgctgaaatatgcgcgcacacatcctcaagccttcccttttatagtagtagcccctttgtgtcccccgggacgccaatgggagccgttggcgttactgacattgttggatgaaatccagcgccagtatcgcatcaaccaacacgccatttacctcacaggctacagcatgggcggtgccggcacttgggccactgccatggcggctccagaccgctttgccgccatcgcgccattgtgcggcagggtcattccactgcttgcaggcaacttgtaccgcacgcccgtctgggtctttcatggtgaaaaagaccaggtcgtgcccctgaaccactccaccgaaatggtggacatccttaagaatcgcaataacccccacgttaaattttcactgctgccgggaaaagaccatgaaatatggcacgatatttacgataactccgccctctactcctggttcatggaacaccgccgcccctgacttgagtacaaccccacgccgcgatccatctaggtgaggcaaaaaacaattccccgcgcaacaacacctttgcctccggagacacgccttcacaatccccgctgctccttcctctgcccataccggccaccatgccgaaaagcaaaattcgccccccttataaattttccatttttcttttgacaaccaaacttttcggcctagagtgacctcatagcttggcaaaccacgtggattaacttcgaggtttcccaacatttaaaatttaaaaggcctgcttatgaacgactttaacctgatagaggagccgtggattcctgttatttaccacaatggcaactattgccgtgtcggcatcaagcaggctctttccgatgcccatctcatccgcgacatcgccacctccaaccccatggaccgcgcgggccttctacgctttctcctctctatagtttattggtgccatggaatgccagcccctcaagaaacgtctcggctaggtgatcctttaccggattgcacaccacatcttggtgcaaagacaaattttttctgccttttcgggcagaaacaccgtttttaccaatcaccaacggcaaaccgagaagtaaaaattggtgaactgtttcaagaaattccatccggaaacaatttctggcattttcgacatgtcaccgataaagaacatggatattgcccagcctgttgtgctctaggcctgctgcgccttcctcttttttcagtcagtggattgccagacttgaaatcggggattaatggaactccacccatctatgtctttcattatggcaaatctttagctcaaattctccgaaaaaactggttgccaatgaagcaaaacttgctgggcacgccatgttggatggaccccaccttggttccaccaatcggagaccctatcccgctgttaatcggattaaccgtcccagcacgcaaagtttggctcggacaacccattcaccatggccgttgctctttgtgcgggaacccatcaaaacacctggttttcgaggctcacattcaatccgccggtacgttggaaaattcaaattggcaagacccacatgttgtttatttcctaaaccaacaacaacgaaagtcatgcaaagctcccaatctcgtcgtaggcggtgaatttcaaatggaccgcccttatgccgatcttatggcatgtctcttggaacgggtccaatttcccgcaactgcgacaagccaacttctaattgtcggttttgccacggacaaagctaaaaatatagatgtttgggagagaactgtaactttggtccttaacgaaatgcagacaaccgcagccacagctttttccacctggtggcgaagtgtaagtgcattgtccaacaatgtttacactattattaaaacaaccccctacaatcggcctcgtgggtccaaaccctcgtatgttcactgccatttttcccacatcagaccccatgtggaacatataatatcatctaacatgcatattctagtaagtgaaccaaacgaaggttggcatgaagcacgccgtgagttcagcgcttttttgccgatgttggctcaatccctctctcccggcgtgacctcccaagccgttcaaaagcgccgtgccatcgctcaggccttgccacctaatccctgcccgaaaccggaaccatccagttcaccctccactcaaaaccccaacttgtaattatgagcgccacccttgaatacattacaaaactgtcccatataaaggccggctcccgcactttggttcgcactcatgccaatctctcattgaaccattccgtggatgcctttgatttgttcaccggtctttggtggcccttgcgccaacaatcacaatttgcaccccgtcgccgcgtggcctggctggtcgccaaacttcacgcagtcagtcctctgccacaccaaccaggccagaccctccccatccagctcgcgaagatttggcataggcagccagacaccaatacaaaaaacaggttccagcgcaaattcgatgatctcctttgccgtcccctggaccaaatggaacccgctttgcaatgggccttgagaacaattggctctgcaaatgagccatccttggactgggtgcaactcaccgacgacctctcccggtgggaacaaccgcaagttcgcgaaaaatgggccgaagtctttttaggcaggtaaactcaaaaccaaaaaagaaaggagcatactcatgaacaatcaaaataccacacgcatacaaatccatctgcttcaaaaccacgcaccggccaacttgaacagagacgatttgggagcaccaaaaacctgctacttgggcggcgtcctccgctcccgcatctccagccagtgcctcaaacgcagcatccgattgagcccctacttcaaagacctctgcggcggtgttcgcacacgccgcctcgcaaaactcatcactgaccaggttgatggcaacaatgcgacgaagaaaaaagtggccgccatcctgaagaaatgtggtttggaggccaaagagaacgacgaaacaaatatggtcgtttacaccacaaaagaagccatcagggaaatggcaacagccttgagaaacggactcgaagaacccgacattatcgccagattcaaagagcttttgagcacgaatactgctgtcccggacatggcactgtgtggacgcatgttggagacaggagaaatcaaggacaccaacgtcgaagccgccctgcaagccgcccacgccatttccacacacgaggctcgccccgaagtggattactatgtgacggcggatgacatccccggaactgatgccggcgctggttatgtggacgaagccatattcgtttccgcctgttattacaaatacttttccattgattgggcacaattgctccacaacctcaagggctacccgggcaacagaatccatctcgcagcccataccgtcggcgcctttatcaaggcagctgccttggtgactccatccggcaaacaaaacagctatgccgcccacaatccacccgatggtatcctcgtggaaatctcccccacacccatcaattatgccaatgcctttgccaaacccgtggacattggccaacgtgatttaatcggccaaagcattgctcaactcgcccagtacatccacgagatggatgccggtttcaaagtcgttgacccgtccaagcgcttctggttttcgccgggcatgcgcaatcgcttggagacctcacagcgcgccggtgatacagcaggcgaccgccaaaccatccgagtccaggcggtggaggttgatcaactcgaaaacctgatccatcaggtcatcaaggccattgacccgatcctcgactggcaggaagtccaaagaaccattctcaataaccatgaagcctgacacacttcttcttagactggaaggccccttgcaagcatgggggcattacgagtccaaatttgccatccgccgtgccgccgaggcccccaccaagtccggtgtcatcggccttctgctggcggccctcggcatcccgcgcacctccgctcctgatgattggttgggccggttaaactccctcctcatgggcgtgcgcgtggaccgtccaggtgtccgttggtgggactaccacacagtgggagctggccttaaaatgagaacagccgagggcaaaaacaaggatggccccctcctcacacgcagagaatttctctgcgatgcctcattcttggtagcccttaatggagagcccgcccttataaaagaactataccaagccctccaacaacccaaatggactctttacttgggacgcaagtgctgcccgccttcaaggccaatactggctcacgcccccggctgtcacaaagatctaccatcggcactacagtctgttccatgggaaaaacggtatgcagacgacaccacaccggaaaaacttgaagccctgctggagtggcgtccctccgaccaacaacccaatgcccccgatgacgccgaaatctggtatgatgccccccaatgcttggaacctcccgctcacggtccccgttttattacgagaactgccttctcagtcgctccagacggccaagtcaccgtggcgccccaacaccaacagcagcttcctttgccgcctccacgcccaagagcaaattacaacaactcagcataccaacgcgctcgtgataagcgtctgcacgccgatcatggcctgtgcgtgttttgcaaaaatcctgccaccaccgtccaacacatcacctaccgccgcgccggtggcaacgaaacaacagaagacctccgctccctctgccgcctttgtcatgatgccgtcaccatgatcgaatacggtttgggtttggagatgggccgcatcaacccggaagacccccaatggcgcgatgccatactcaagaaacgcaccgaaatcatccaattccgttcactcgaaaaccgcagacgttttttgcaaccagaggaggtttaaccatgtatttgagccacttattgattgatgtcggcaccaaccccgaccggccacgccccggccgactttggctgcgcaatatttaccatgtgcaccaacgcctgagcatggccttccccaccactcagcggaaaaccaacgacccccacttcttgaaaccctacaacccggccgattttcccctgcacaaccttggcaaccatgtttcccgcaacgaaaacaccggatttctcttccgcattgaccccgtgccggggggcaatcccctgatcatcgttcaatccagcctcaaacccaattgggactacgccttcaacaatgcggcattccttctggcagcccctccccaatgcagggacttcctgcccaatttcaacaaaggcgatcacctgcgcttccgcatcctcatcaaccttagcaaaaaagccaggacaagccatgacggcacccctctcaccaaagtcaaagaaaacatcctcgataaccaaaaccgccctaaatcccagtccaaacgtgtcgcgctgacatgggatgcaaaccaaaatccacaagatgtcatcattccttggttttcccgcaaggcatacgtcacatgccaaaaccaaacccaacccatccaggcatttcatctcaagcaatgtgaaatcctgcgcatgggttggattatcggatggaaaaaccaccatgcagccccggaaccgacctcacaaaaaatgaaattccgctctgcccttttggagggagtcttggaagtttcccatcctcaagccttcgccaaactagtagcctcaggcatcggctcggccaaagcattcggttttggtttgctctcgctctccccggtttcctccgctcaatgaaactggacgacctccatatcctccccaaattctccgatggttggagctacctctatgtcgagcattgtcgcgtcgaacgagaacaaaacgccatcgccatctatgacacagatggcatgatccccgtcccttgcgccaacctggctctcttgatgcttggcccgggcacctccatctcccatgcagccataacagttctggcggaccacggctgtttaatcatctggtgcggagaacaaggcgtccgcttctacgccaccggtatgggtgaaacccgcaatgccgcccgcctcctccatcaagccttcatgtggagccacgaaaacctccgtttcgaagtcgtccgccgtctctacgcaatgcgttttacagaaaccctgccatccaacctgtcattatctcaaatccgcggactcgaaggccgccgcgtccgcctggcctatgacaaagcggcccgtgccagcggcatcccctggaaaggccgccagtatgaccgccatcattggacccactccgaccccgtcaaccgtgccctttccaccgccaattcctgcctctacggcctctgtcactccgctattgtctcctcaggtttctcccccgccataggctttatccatactggcaaaatgctctccttcgtctatgatatcgccgacctctacaaaaccgaaatcacagtccccatagctttccaagcggccgcccaaggagacaccaacctcgaacgccgtgtccgcatctcatgccgtgacgcctttgccgaatctcacctcttggcccgtattatccctgacattcaaaaagctcttgcttggcaaccatcccaaaacgaaaactccccccttgattcagaccccgcccttccctctcacctttgggacccggatttaggggccgtgcaaggcggaatccaacaacctgccacatcccctttgcccccaactataccagccccagccccggaatctcatgatggtgctaatccttgaatcagtcccaccttcactgcgtggagaactcacccgctggctcttggaactcaaagcgggtgtcttcgtcggccgcgtctccgccctcgtccgtgagcgcctttggaataaatcctgccaccacgcccgcaatggcggctgcctcctcatctaccacaccaataacgaacaaggctttaacatcctctcctatggtaatacatccagaactatcgccaacttcgatggactcctcctcgtcaaaatcccaaaataaccccattttccaagggttttccccacgcacgtgggggtgaaccgcgtggtgcgaggtgaacaagaagatgccaagcgttttccccacgcacgtgggggtgaaccggccgtcacctgcccctcccgatccgtccccacgttttccccacgcacgtgggggtgaaccgcggcaggtggtgaagagttcggcgaagtttgcgttttccccacgcacgtgggggtgaaccgtccacccccatcaccagccgccgccccgccgcgttttccccacgcacgtgggggtgaaccgcttccgaagttctcacggacgaagctcaggcagttttccccacgcacgtgggggtgaaccgggcatatttacggtttcacaatgaaccaactcgttttccccacgcacgtgggggtgaaccggccgctttcgaggagctgacgcggctgaagaagttttccccacgcacgtgggggtgaaccggtggcgcgccggtcaatgcagtgccatcttcagttttccccacgcacgtgggggtgaaccggggacagaatatattgtcgagccgctcaacctgttttccccacgcacgtgggggtgaaccgtccctgaccgccatgaactccacgcccgcgccgttttccccacgcacgtgggggtgaaccgccattttgatcaaattccctgcacctcaatccgttttccccacgcacgtgggggtgaacctgaggttgaggccatccgccggcgctcgaaagcgttttccccacgcacgtgggggtgaaccgatttcgctgcggctatcggcctgaacctcaaggttttccccacgcacgtgggggtgaaccgaacctggccatcgagaagaacctggccatcgagttttccccacgcacgtgggggtgaaccgtagtctggccacagcatttcttagagacaactgttttccccacgcacgtgggggtgaaccgtcgatatagccgggttttccagcttcgagggagttttccccacgcacgtgggggtgaaccggcatcgcgctttgatttcgaggaaaaatacgagttttccccacgcacgtgggggtgaaccgcctgtgcgccggtctcggtctgaagttaatacgttttccccacgcacgtgggggtgaaccgttagtcgttgtctcaccgccaccgctcgcgctgttttccccacgcacgtgggggtgaaccggcctcattcccggttggcttaaaaacggtgaagttttccccacgcacgtgggggtgaaccgtgacttggagccaccaacacacaagccagcgcgttttccccacgcacgtgggggtgaaccgacgattgcaacagcggcgttttcggacgcttcgttttccccacgcacgtgggggtgaaccgtggaggtctgaggatggcgagcacgttgggccgttttccccacgcacgtgggggtgaaccgattgttgaaaaccagagccggagcattaatacgttttccccacgcacgtgggggtgaaccggatgtactcggtgccggcgatcgaaaacgggcgttttccccacgcacgtgggggtgaaccgtccaaaccgataagaacatgaagaaacacgaagttttccccacgcacgtgggggtgaaccacgcagcgcggcgacatagtggtgttcaatggcgttttccccacgcacgtgggggtgaaccgcaatgaggtccgagggctggtcatcaacaagagttttccccacgcacgtgggggtgaaccggtcattggcactcttgaccggcgtggccacacgttttccccacgcacgtgggggtgaaccgctttccaggcaccacatcaacaattccccgaagttttccccacgcacgtgggggtgaaccggaactggtggaaatctctacgttgcgcttagtgttttccccacgcacgtgggggtgaaccggcaagcctgacgccaccaccaccgccgacggggttttccccacgcacgtgggggtgaaccgcgccgccatggccggtcatctggccggccaaagttttccccacgcacgtgggggtgaaccgtgtgatccgcaggcgccggccccaacggcggtgttttccccacgcacgtgggggtgaaccgtgatgcccgcggccgcgggcaatgcagtggccgttttccccacgcacgtgggggtgaaccggtccagccggctttgcgcggggccggcgaggggttttccccacgcacgtgggggtgaaccggttgccagacaatgttgacgtagccacatacagttttccccacgcacgtgggggtgaaccgtttgttccagtgaaataccagccaccgtcactgttttccccacgcacgtgggggtgaaccggcggcgatggagctggcgcgggttgaggcggagttttccccacgcacgtgggggtgaaccggtctgcgccagaaccactagctcctaaaatatgttttccccacgcacgtgggggtgaaccagttagttccgcggcccccgtgttcgagacgcgtgttttccccacgcacgtgggggtgaaccgctatgctggccaatgtaggtatgatgagtgatgttttccccacgcacgtgggggtgaaccgtttgtttaactctttagaagaaaccgaaatgttttccccacgcacgtgggggtgaaccgaaaccgaaatggaacgcattctgcttgaacacgttttccccacgcacgtgggggtgaaccgaggtcgaagcggagcaaaaacgggccgctgaagttttccccacgcacgtgggggtgaaccgaaaattattctctgacgtcatcatattctgaagttttccccacgcacgtgggggtgaaccgtttgagtcggactgatgtggtggcggacagtggttttccccacgcacgtgggggtgaaccgttttggatcatccggcgagtaaagaagttttcgttttccccacgcacgtgggggtgaaccgccaccacctccacgatggtgcaggttggaaaagttttccccacgcacgtgggggtgaaccgagggcggcggcattgccgcacgccacgagtgcgttttccccacgcacgtgggggtgaaccggcataaccacgagcccactcttcaaacttttcgttttccccacgcacgtgggggtgaaccgatcgtccagctttttgaagaagcggacgagatgttttccccacgcacgtgggggtgaaccgaaacggctttcgccgcaacgaccacaatctaggttttccccacgcacgtgggggtgaaccgctggtggacacgacccgcaaggaacgcaacgcgttttccccacgcacgtgggggtgaaccgataccacactgtctgccgacgaagccgcccgggttttccccacgcacgtgggggtgaaccgcagattgcggtgtggttcagccgtcaccaaccgttttccccacgcacgtgggggtgaaccgtttcatggcaccgaagaccacgatgtggacaagttttccccacgcacgtgggggtgaaccgaagggctggcgtgccggtaagtaacatcatcagttttccccacgcacgtgggggtgaaccgatggaaccgtagccacggagtttgagtttgttgttttccccacgcacgtgggggtgaaccgggcaggcggacagattgccacgcctcgcgacagttttccccacgcacgtgggggtgaaccgttgttcggcccagcgctccggcagcatccggtgttttccccacgcacgtgggggtgaaccgctcggtttcacgtgcttgtcgttcacgcaaaagttttccccacgcacgtgggggtgaaccgcggtactgacgggctggtctcacgcccaagccgttttccccacgcacgtgggggtgaaccgaattgcgtataatccggaaaataacacacggagttttccccacgcacgtgggggtgaaccgaagcggaggccaaggcattggctgaagctgaagttttccccacgcacgtgggggtgaaccgagacggcttttcagccgcaacgaccacaatctgttttccccacgcacgtgggggtgaaccgatgcaatctcggctgctacgaagcgtggcacggttttccccacgcacgtgggggtgaaccagggggcaccttgaaaaggttgacatctgtggcgttttccccacgcacgtgggggtgaaccagtagtcgcctagttggattcgttgccgaatccagttttccccacgcacgtgggggtgaaccgctggtggacacgacccgcaaggaacgcaacgcgttttccccacgcacgtgggggtgaaccggcgcacataaagtgcgccaatgaacgcaagaggttttccccacgcacgtgggggtgaaccgctctttgccacagcgcttgcggccagggcagggttttccccacgcacgtgggggtgaaccgaaggaaacagcttccaaaatgccatccttgcggttttccccacgcacgtgggggtgaaccgtcttatgccagacccagacaacagccacgaaagttttccccacgcacgtgggggtga

SEQ ID NO: 8 - DNA, 28258 bp, unidentified
  misc_feature 1..28258: note = JAAYVF010000044_organized
  source 1..28258: mol_type = other DNA
aaatcaaggcgaacgagaacgactgcgcgagtttcagaaacgcctcgcgcgcttcctggacggtttcggcggatttgtcgtacagaaacgggatcaaatcgtccaactttttgttttcagcgagtttttttactttcgagccgccaaacagccccatgatatccccctgattcttttctgatcgatcgtattttccatccgtctgattggttcatcggactgtcggtcatacacctcaagtattcatgaagcgaaaaaaggcggggcgtaaaagaagtcaaccgtcgccggccggcctaatggccttccagctcgatcgacagcatccggtcgccgatcacgatgtcctcgatgaccgccagcgatttttcatccgcctggccgaaagccgtataacggccgtcgaggtgcggcgtcggcaaggcggtgaaaaagaactggctgccccccgtgtcgtggccggcgctggccaggcccagggttccggccgcgaacggaaccgcgccttcctcatcgcgcaccgagtagccggggccgccaaaaccgaggccttgcgggtcgccggtctggatcacgaaacccggcaccacgcgatggaacacgaggtcgttgtaaaaaccagtggtcgccaggcgcaggaagtggtcgaccgtcagcggggcgcggtcgcgccgtagcgtgaagcgaatgtcgcccttgttggtgcgcaggaccatctggagtggcggttcgttttgcaggcagcgtttcgccagggcgcggtaatcctcgagcgagcggcccgtcgcgacgccgtacagctcctttttgttgatctcgcgcgtcagcttgtgcagcagttcgacgcccttgcgccgcacctcgagacgcttgtcggccagcgattcgcgcgccatctggctgaccgccacgctcgcgccgaccgccaacttgtcgtcgtcggtcagcaaatcgagcgatttcaggatttccagggtcgcatcgggcaacgagtcgtgtttggcccgcttccaggcggcgaccagcgtcttgaaggatttttcatccttgccggcgatgagcgaagccgcggccgcgccgcgaatcaccggatcggggtcttttaaccactccgggtgggcgcgatacgcctcggggcgcgctttgccgtcggcgtcgtacaccagcgccatgaccagccgttgccgcaccggcaggctgcgcacgaaccgctcgggcaggctcagaccggaggccagccagttcagcttgtccatcagccccgccgcctcgagcacttcgatgtagtgaatcgcgtcctcgtccgcgccggcgtactgatcggcggacaggccgttgatctgctctttcgcgccggcccgcgccgccgcgatccgcgcgaggcggtgatacggcaccttctgccgcgaaacgtcccacaggaattcggcgacgcccttcgccttcagcccggccatggtttcgagcaccgcctgatggacgccggtcgaccggccggtcccggccgcccgcaacgcctgctgcagcaggctaaaaacttcctcggggggcaacagcagattcaccgcctcgacggcggcgattttttcctgcgtcgtgccttcggccaacagccggcgggccgtcgcggccaccgcctcgtccttgacctgccggcgggcgaggccgagcagcacctgggcgcgcagcaacggttcgtttcgcgtcagcacctgtttgagcggttcggtcagattcaactcgcgggtcgccggccgggcggcgaagtaagccgcgaaatacgcgacatccgcgttcggatgggtcagcccgaacgccagcactcgctgccgttgttcgttgcgttcctgcacgagcagcggcggttcgagcggcagttcgatggcgggcgcgccggcgacgcgcatcagcgcctgcaagagtggcgcggggtcgccggtcagcttgccggcggccacttccgactgcacggccgccgcctcgcgatagccgtgacccggaaacgccagggcgatcgcctcgaacgccgcggcccgcgccgccggatcggggctcttggtcgcgacgtccagcagtttgcgggggatgtgcggcagaatttcgattttgtaaagcagcgcgggggtattggcccacaactcgcccaacgcccggcaaagaacagcctgattcaccttgccgtcatccagcgcccggagcagcgccggcgagccttcgggatcgccgatgtggccgatggcctgcgcggcccattcgcggacgcgcatctcctgtttggcgtcggccaaaatatcgcccaggaaagcctggtatttcagggaatcctcggcttgcagaatccgggccagatcatccgccggctcggccgccgcgacggacgaaaaccacggaacgaaaaccagcaacaaggaaagaaccagccagctcgtttttggcgaccccacgattatcctccaccaatgtttgtccgagaatagtgcggcgtcacggcgctttggtcaagcaggcggcgggataaaataacggaaaaaggccgaatcggcgctattcgtcctcttgttcggcctgcaattggatcaccatcgaccgaatgtcccgggtgaacttcaccgtgtgccggacccgttcgatgtgcagcggcgtcgggacggtgccgccgatgaccgccacctggccatacacctccacctgcagttggctcattcgcaggccggagattttcgccaggcggcgcaacaattccttgcggatcgcttcgtcgatggcgatctgtgtcagcagccgcggatccttttcggcgaccggcgggcggaaccccggcgccagggtgattttgcttttcacctcggcgactcccggcaaggtgcgggcgaccgcctcggccagcgagcggtcctgcagggtcttcacccggccgctcattgtcaccacgccgcctttgacctcgaaggcgatcccctgcagatcggcccgccgggtcgccgccaaacccgcgtggattttttcccgcaataccgggtcgggcactattttcttggcttttttcagcgtgcccggcgcgggttcggtcgcttcgggagtcggcgccggggtcggtgtcggcgtcgggttgacggccggatcgtgctcgtcggtggtgtcatcgtcatcttgagcctgccccggacttgatccggggtcatcgtcggcccaaagcggcgcggcgaggccggcgagcgccaatccgcacaaaacgaaaacgacggccaagcggttcatggacgaaaagtgccaccggcgcgaaagttaggcaagcgttttttcgcccgggggctctttccaaccgggccgctttctgttaactaaagacaacatttccagcgaggtcttccgtgatccgtcgtgcctgcctgtgtctctcgcttttcctgctgcttccggttgtcgccctcgcgcagacgttgcccaacccggaggatctgccgaccggtccgcggcccgcgagctggcatctgtacaacggcatggcggtgggcttgaacggtccggtcttcggcgacctcttcaagttctatcgcaaaatgccctggggcgaaggcaagaccctgctcacttccgactcgcactggcgcgtgggcacgcacgatgtcgtcagcccggcctacgcccgcccggcgatcctgatcggcgtttcgccgctgttgatcatggacctggacgtgcactacggccccggcatcagcttcacctacgaaaaatacaaatcggtctacgccaattacgatccgcaaaacctcggcgatcccgacggctacatgaaaatctttcaccaggcgcaggccaacctggtgctcaaggccgccgtcggccccgtcgcgatgctgcacttcgacgacctggattacttctactgcgacgaaccgatcttcctgtgggaagtcgccaccgtcgtcaaggacggcttctttctgcgcgacaaaacctacgtgctgatcgagttccagcccaactggcggctgctgctcgattacgaaaacttcgagtacttcaagtcggactacaaaaccgaactgctcgccagcggcatcgtcatccagaacccggccttccacaacctgacgatgatcgtgcaactgggctaccacctgcgaaaccccgatttcaaagggctcaaaatctggtccgccgcccagatggaatgggatttcccggacaaaaagaagtaacggccgcgcggccggttttcacggccgcttttcggttctatattttttcatcaatttcgtccaatttcagtaacgaacgacaccaaacacggcaaaatcggttatcgtggaaccgatcacaaaggagattcccatggccgacgaacccaaaacgatgattctcaacaacgatgtcgcgatcgaggcgttgaaaaaagtgggggcgatgaaaccctgtccgcgttgcggcaacgacacgttcaaaatcctgtcgggattcttcaatccctcgattcagcagagcaccagcggcgtggtgctgggcggcccgagcctgccgtgctgggtgctggtttgcgcgcggtgcggtttcgtctcgatgcattcggtggaggcgctgaccggggtcacccagatcgagccgatcacgcactgatcccggccggtggccgcgccggtccgtccccacttcttgcgaaccaggatcgccggcgggataatagcccgatcggcgcctcggctttgttcgtccagaaaatgaaccacacggagcggaacatgcgaaccacggcggttttcttcgcggcgttggcggctcttctctttttgttcgcggcgatgatcgcctgcgacgacgacgacgataatgacaatgatgcctccggcgtcgacgacgaccagtcgaccggcgacgacgacagcgccgccgatgatgacgacgacaacgacgacgacgactcggccgacgacgacaacgacgacgataccgtgcacccgtcgctgatccccggcccggacgacccgggttacgacgaggatctggaagccgcggcggagcagcgcgagcggcagttccacgtcttcaacgcctacgggatggacatcagcgccgacgtgtcggtgtcgctcgacgagcccgcgaaccgggcgctgatcgaggacttcattctcaattccgacagttgggatttcgcggccgtcaccggcctggatccgttcgacgtggtgaccgaatggcagttcaccgcgggcctctattccggggtcggcatcgccgccgacgcttatcgttacggggtgttgcgcgacgagggttatccgcaagcggacatcgaccgcgcgcgcgactttctgcaaaaggcgatcgaggcgctgaaggtcgcctacctgatcaccggcacccccggcgtgatcgcccgcggctacgtgcgcaccgacatccccggccccggcccgggcatcgaaacgacgccgctgttcgacgaggaaggcaaccccctgcccgccgaaaagaacaacggcaccaaccgcgccgacaattcggtgggcggcctgtatccgaactacgtctgggtcgactcgtgcagccgcgaccagtacatcggctgggcggcggccttcgcggccgtctgggaagtgatccgcgacgacccgtcgttcgacgacgacctgaaggccgacctgcaggattacgcgcgcgaactggccctcgccctgtcggtggtgcgcgaaagcggcttcgacctggaaatccccgacgccgacggccggacgacctaccacggctacctcaacgaaaacaactacgaccgcatctacatcccgtggctgccgatcaaaaacggcatgtactcgctgatggccatcggcatcctgggcgctttggcctacgtggccgaggacgaggaagtcgatgcctacctgtacgacacgctgatcgaccagcggcaactggacctgatcgcccgccagaaccagatcggcgtcgacctgtgggtcatctccaactacagcagcgtcaacatggcgtttcagggcgcctggctggcccatcgctacctcgaccggccgcgttcgcgcaacctggtcaagctgaccctcgccgagcggctttacgacaagccgatgtggcatcagcggcaggccaaggaaatcaagcagagcctcttcgatttcgtctacgcggccagcctggccgacgccagcgtctacaccaccatgtccgccgaacccgacgccgaggcgatggcccacggcctggaaacgctggcggactttccagtcgcgccgttctgggaatacggggtcgtcaattgcgacgaggacgaaatcgccgccggccattgcctgtgcgtcgacggcgaaaccgaactcgacctgatgccgatcctggggcgcggcgacaaggtggtgtcggtgcagccggtgccgatcaaggtgcggccgacgagcaattactactggcgttcgaatccgtacgaggtcaacggcggttcggacggcacccggctgctgccggccgtcgatttcctctatgcctattggctggggcgctggacgcgctgagggcggtcaatcgtcgaaccgtttctcccgaacgtggcagtagggctcgccgccttttttctcgtaatagtcctggtggtaatcctcggcgggccagaacgttccggcgggcttgattttcgtcgccacccgatagcccttggcggtcaacaagccgatcagtttctcgacgatccgcctttcctcgtcgttgttcacgaaaaccaccgaacgatactgcttgccccggtcgggcccctggcgatcgacctgggtcggatcgtggatctcgaaaaacagccgcgccagcgtctcgtaactgacgcgcttgggatcataggtgacacgcaccgtttcggcgtggccggtgtcgcccgcgcagacctggcgataggtcggcgcattcacgtggccgcccaggtaacccgattcggcgcgaatcacgcccggcacggcttcgaaatggtattcgacaccccaaaagcagccgccggcgaaataggcggtcgccgtcccgtcggccgtcgccgcgggttcggcatttttcgcggcgaaatcgatcgaaaccgagttgacgcagaaccgggcgtcggcgggggtgaagccttcgccgcggaagatgtggcccaaatgcgccccgcaattggcgcagacgatctcggtgcgaatgccgtccgggtcggggatttccttgatcgcgcccggcaaggcctgatcgaagctcggccagccggtgcccgaatcgaatttcgccccggatgaaaacagcggcgtgccgcagcgcttgcaggtgtaggtgccgtccgcgtggtgatcccagaactcgccgctgaacggcgcttcggtgcatttgccgacgatcacccgctcttcccgcgaagtcagttttttccagttcggatccatcggtttgctttccttcattttgccgcaggcgccggccaagagcggcagcagcagcacgatccataacaaccatacccgccggggcatgacaatcacctcgtcttttgcgaccgcctgatacggtcgttggataaatataagaatcgttcctgataagcgcaatgaaacgatgccgcttcgattttcaaaccttcgtgaccggcttatgatttgcgtctcgcgatcatcaccgaaagggaggctttcgatgcccgtgcccgaaaaacaacgcctgccgcgccaggtcaaagcgctggggctggtcagcctgctcaacgattcggccagcgaaatgatttttccgctgctgccggcgttcgtcacttccgtgctcggtttgggcccggaagtgttgggcatcatggagggcctggcggagacggtggcctcggtgctcaagtacctgtcgggctggtggtcggacaaggtgcgccggcgcaagccgctggcggtggccggctacacgctcagttcgctgttgcggccgttcatcgccctggcgacggccggttggcacatcgtggcgctgcgcgccttcgaccgcgtcggcaagggcattcgcacttcgccgcgcgacgccttgctggccgaatccacgacgccggaaatgcgcggcaaggcgttcggcttccaccgggcgatggaccatgccggcgcggtgctcggcccgctggcggcgttggctttgttgaacctggggcgcctcgatctgcggacggttttcctctggtcggcgattcccagcctggcgacgattctcttggtcgttttcgccgtgcgcgagccagccgccgtgcccaaggccaacgcgccggtcgccgctagccccgcccccgaccgggagaaaaaccggcccttcgtcgtttttctgctgtcgatgatgatcttcacactggggaactcgagcgatgtgtttctcgtcctgcacgcgcaggaactcgggatgcccgtcgcgctggcgccactgctctggatcgtgctgcacctcgccaagagcctgttttcaacgccgggcggcgcgctgtcggatcggatcgggcgcaaggcgacgatcctgctgggctgggccgtttacgcgctggcctacctcgggttcgcgctggcgaaccaggcctggcagatctggccgctgttcgtgttttacggccttttctatgggctgaccgagggcgccgaaaaagccctgaccgccgacctgattcccccgcggttgcgcggccggggcttcgggttctatcacctgagcatcggcatcgcgtcgctgcccgcgtcggtcgcggcgggttttctctggaaggcgtacggggcgccggtcgcgctaggactgggcgcgggtttcgccgggctggcggcactgattctgggcctgggcctgccggcggcgaaagccggcgaataatctcggatgtctcgcttgattgatcggatcattccagccggtaacattcttaccggaatctattacagatagcgtccatgcgattgacagatggagcattgacagggccttttaccggtaaaatgtctgattccgacggcaatgccatcttttcgatcgatgcgattttccactaattcccataaatatttttcatccttttcaatatacctgcgcagggcgtaggcacaaaggtcggccatttgaatcatactggtcaggcagctatcgacaaaaaagggcgtttcaataatgttatcgatcatccgaaaaaaagttccggagcgatgaaattttctcatcaattggtttaggcgaggcgccacagtggaattattatcctgaatgactatccccttttcgtcgaaagcggccaagaaggcctgaaacctggtaagaacttgttcaaaagcgatttcatacggctgatttccttgaatggtaaaatgcggttttgaaatcgcttccgcgaaaagccgaagatatccccatctgcccaattcagccgccagatcgatcaaacatttttttctttcatcaaaggtaaggtggatatagggccgtattttactcacttcttttcgataattttttatttttttctgatttccctggatgccgatttgtccggccctttttcgaatattcctttccacctccattcgtctctcattccaggataattttccgaaatcggcaagattgctttgttcgacataccgccgggccatccaagccgtatggatttcctgattttccaactgatatttttgttttatctgaagcaagttcaggtccaaacttttccatttctcggctggaatcgccagtccaattaaaatgaaatgatttgtcccgcccgttaaatcctcgattcccgattcatccacatagagaaaatacatcctcttgctccattaaaaaaaaaaagcgtctagagcgaagcctgatggctccggaggggtcgcatcgcgtccccgtctctagtcgctgatttattaatgcccattattataaataaagtcaagaattatttgccccaacataattcagcatcatgataatatggaaagggagaaaaggaaaattattaaagacgctccaataaaaatcctcctttatttgcaatcactttaacaccttgacgaaaaaggccgcacaccacaatatatggtgcaccaagtaaaagacgcatcaactattagcgatttttctttgacttgcatcgttcggtgtgctaggaaatgggccggcacaccttcggggtttcagctggggacaatttgaatgcgaattaaagacctggaattagtcggttttaaatcgttcgtcgataaaacccgcttcactttcgacgacgggatcagttcgatcgtcgggcccaacggctgcggtaaaagcaacatcgtcgacgccattcgctgggtgatgggcgaaatgagcgccaaaagcctgcgcggcggcgaaatgcaggacgtcattttcaacggttccgacacccgcaaaccgatgggaatggctcaggtttccgtcattttttcctgcgaggacggcatcaccccgtccggctacgaagggatgagcgaaatccaggtcacccgccggctgtatcggtcgggcgagtccgaatacctgatcaacaaacgcgtctgccgcctgaaggacatcgtcgacctgttcctcgacaccggcatcggctcgcgcgcctactcgatcatcgaacaaggccagatcgccaaggtcatttccgccaagcccctcgaacggcggattctgatcgaggaagcggcgggcatcagtaaatatcgcgtcaaacgcgaggaggcgctgcgcaagatcgaggccacccaaaccaacctggcgcgggtcagcgacgtcattttcgagatcaaacgcaccctgaccagcctccagcggcaggcggccaaggccgagcgctaccacgaactcaaggccgaactgcaagaactcgacgtcgaactggccggccgcgaaaaagtcttgctggcgcggcaggccgagcagaccctggcgaacatcaagcacctgggcgacatgcaattgcaggccaccacccagctcgaggtggaggaaacccagctcaccaagctgcggctggacgtgctggagcacgaaaaggcgatcaacctcgccgccgaaaaagtcgcccggttgcgcgaaaccgtgcgggccgacgagtcgcagcgcgatctgttgtcgcgcgacatcgagcacctcgaggcgcagatcggcctctggcgcgaggaaatcgaggccgcgcgcgagcgcatgggcctgatggatcaggaagtcgcggcgaccgtcgaaaaaattgccgacctgcaaacggccgccacggaagcgagggcgacgctcgcccgcaaccaggaaatactgcaaggcgcccaggcgcgccgccaggatttggaccgcgtggccgaaagcgcgcgcaacgaactgctgcgcctcgccggccggcaggcctcgctccggcaggccgtgcagaaccaccaggacaaccgcgccgcacggcagcggcaaatcgagcaaaaccaggcccgcgcggcggctaacgaggaactgctcggcaagaccctggccgacggcgaagccctgcgcgccaaactggcggcgctgcaggaggaacgcggcaagctggaaagcgacctgggcgacaaacggacccggctcgcgaatatccggcagaccgccgagggtaaaaagaaggccttcaacgaggcgaaagaagcccacgacaaaacccgcgtccgcctggaaagcctggaggaaatgcggcgcaacctcgaaggctatcagtacggcgtccgcaaaatcatggaagcggtgcggcagggttccggcgggctgaacggcacgcgcaaggaaatcctgggtgtgctggccgaaaaaatcagcgtgccggcccggttcgaaaccgcactcgaagccgtcctcggcgaacgcctgcaggccgtcctggtcaccgaccaggaagccgggcacacggccgccgattacctcaaaaccgaaaaggcgggccgcggcagcttcgtgccgctcgaaccgcgcctgccggccaagatcgactaccccgaaaagaccatcggccagaccctcggctgtctggtcaatcacgtcgaactggagcccgaataccgccaggtcgccgaggtgctgctggccaatgtgctagtggtggaaaacctcgacgcggccatccggctgcacaaggaaaacggctacaccggttcgttcgtcaccctcgacggcgaactggtcgacgacaccggcacgatcaccggcggccaggtggacgcggtgaccagcggcatcttgcagaaaaaacgggaaatcgccgaactgtcggccaaggccgaggaactggctgccctgctgaaaaaagccgagggcgactactatgccagcgaaggcatgatcagccgcctgcaggaggtcatcgcccaaaaccagcaggcgctcgacgacaaccggatcgccctgtcggaaaccgccggcgaatcgcggcggcggcagcaggaaatcgaacgcctgcagacagaaaaagccggcttgcagcaacaggccgcgcgcctggccgaggaactggccgaactggaaaaacgccacgccgccgaggagcgcgaattgggcgaggtcgaccaggcgctcgccgcggcgcgccacgccgccgacgaaaaaaccgccgcccagaaggcgctcgccagcgaaatcgacagccaccagaaagccgtcaccgaatccctgctggcggtgaacaacctgcggcaggaaacccacaacaccgaaaactaccgcgccaaccgcctgcacgcgaaggacgaagccgccgctctcgtggcccggcgcaacgaacaggtcgagcaagcgcgcgaaacacagttgcgccaccgcgaaaaaatcgccgtcttcacccaaagcatcggcgagaagatcacccatctcgaggcggccgagcgggaagcggtgcaggtgcgcgagggcgtcgacgaaaaaatcgcccgggtcaaccagagcgaaaacgtcatcaagatgctgcgccgcgacctggaaagccacgcgcgagagatccactcggccgaactgaccgcgtcgcagatcgccatgaagcgcgagcacgtcgccgagcggctgcgggaaaaataccagatcgagctcgacaccctgcccgaaccggccgacgaggccgagatggacaccgaggccttgcgcaaccgggcgcaggagatcaatcagcgcattcagcagatgggcgaggtcaatccgaacgcggtcgaggaatacaacgagcagaagacgcgttacgaccactacgagacgcagaagcaggatctggaaaacgcgatcgacgaattgaaacaggcgatcgccaaaatcaacaagaccagcaaggagcggttcgaggaaaccttcaacctcgtcaaccacaaattcggcgaggtcattccgctgctgttcggcggcggcagcgcccagctcattttgaccgagccgaccaatccgctcgaatccggcctggacatcgtgatccggccgccgggcaaaaagctgaccaacgtcaatctgctctccggcggcgagaaggcgctggcctcgatcgggctgatcttctccatcttcctgatcaaacccagccccttctgcctgctggacgaggtggacgccccgctcgacgacgccaacgtcacccgcttcaacaaactggtgcagcagatggcgaaccacagccaggtgttgctgatcacccacaacaagacgacgatggaagtggccgacagcctgttcggcgtcaccatgcaggaaaaaggcgtctcgaagctcgtcagcgtgcaattcaccaacgacggggccgccgccaactgagcgccgcggaattaaatgaaaagccacgccgccgggcgtggcttttttcgttcgcttgggggcggactattccttggtcagggtgtccatgaccaccgaggtcggccgcgtgaagagcaccgtgatatccacccgacgccattcctgatctttcttgcggctcccggttttgccttcacccgtcgcgctggtcgccggcttgctgccgagggcctgcatgtaaatgcggtttttcgaaaggccgttttccgacagaacgaaccgcgtcaccagggcacgccgcttggccaagtccgcgttggcgcccggcgttcccggagcgtcgcagtacgaggtaagcaacaggtagaatttgtgatccttggacatcttgcgcaggtattcgatggccttgtcgagcttctcgcgctcgccgacccggatcgtgttcgaattgtagtcgaagtagaccgaagtcttgaaattcttcaactcgtccggaacgacttcctccactacgggctgggtgggctggcttttgcgggcgatcgcgcatttcttcgcgagttccgccagcttgccggtctgggtggccacgacgccggcctgatcgttctcgccgtcctgcagcagcaactcggcctgccgcagtttcatttcggcgctttgatattcgtcggcgcacagcttatcggcgttttcggccttggcatccagcagcgcctgcttggcgacggcgatttgttccaccgccggcgacggcgccgaggcgcagccgacgaacattccgcaggcaaccaagatcagcagagcaaaagcgatccgcttcatgatcttcctcccggacaaaaaacgagtccgcttggtagtatttcgtttttgcgtcgaaaagataagtcgtttatagcgtgaatgaaaacccggcgcaagcattgtttagtgcatcatttttaactttttgcgcgccagatcggcctcggggctccgcgggaattgttcgaccaccttgtcgaagaacaacttggcgtcgccgacccggccggtttcgtagaaggagattcccatgttcaggtaggcttccggaacgcgcttgctctgcggatatttcgaagccagggtgtcgaactcgaggatcgcgtcctcccacgaacgttgcttgaagaggcttttcgccaggtaatactgcgcctcgctgcacttggggcttttcttgaattgggtgagaaaatcccggaaccgcgatttcgacgcttccaggttgccctggttgaatagcgccaaggcctcgcggtacacgtcgtcctcgtttttcggctggggcgtatccgccgccggcaacggcgccacgtttccggtcaattcctcaaggttgccgcggtcgacgtccttgatgcccgctttcttctccatgaccgacaaccgggtgtcgatcgcttcgaactgccgcgccaactgccgcctcatcagggccaattgctgcggcgaaacctgttgctggccggcattgtggttcatttcgtcgattttgccttccaaggcgccgacctgctgttgcagctggtcgaaattcaacaccgcttcgccggacatcttggtgctttcgttgaaccgatccaccagattctggttgtttttctccaaggcctcgaggcgccgctgcatgtcggcgatgttggattccagctcattgccctggctggtgcggatcgggcagccggtcagggagaacaacaacaccggcaataaaaacaggcccagcagattccattggttcttcatcggtgatcactcctcgccgatccggcgatcggacagaaaaaacacccgtaaaaaagcgggaggatcatagcatttcgtcccggcgcgttcaacttcaaatgcccgcccgcgcccgcgaaatcgaccgctattaccgatcgttcgccttgacaaccgccggcaacaactgcccccttggacacccgcgaccgggctttggtttcccggcccggtcccggccggccggaataaaaaaagctcggaagcggttacccgcctccgagcccgattgtccactaagctcagagcttatttgccgaggtcgacgaattcgacgatacggttggcctgatagccggcttccgtttcggcagcattgaacttcttggtctcgccaccggatttcttttccaacttggccttgtccacgcccgaagcgagcaggaagttgtacacggtggtcgcacggcgttcgcccagcgccaggttgtactcttccgtgccacgaacgtcgcaccagccgaggagctggaacttgtggtcggcgaacttttccttaatcttcgcagcggtgtccttcatcttcgcgagctggtccggacggatcgcatatttgtcatagtcaaacaggatcgcatccaggctcgctttgatcgcggcaggcggaggaggaggcggaggaggcggcggcggcgccggcttgttcatctcttccgctttcttcacggcgcagatcttcgcgatatcatacaggttcttggcgttttggaacagcggcatcgcggtgtcccaatcctcggcggcatccgcctcgagggcctgcttcatggtctgctcggcagcagcccattcggtcgcgcagagttcgttggctttctgggcgctcgcaccggcttgcgcgtccaaagcattttgcttggccacgtcatagccgtcaggcggaccgcaggcgaccaacaaaaaggccgcggcaaccgccagcaatacgaaaatagaccttttcattcgtacctccattggttttagttacgaggttatagtcatttacaattgcgactaatataacgcccaaccggcgttttgcaaacgactaattcaataatctgggagaccaggcagaattcttatcatccgtgcccggtcttgtcaactgtttggtgtaacttccatcgacattaaacagtcgcagcatcctcccggaaccgttattggaagttaatgcgatcgccctgccatccggagaaaagcttggatattcgcaagatccgaagccttgtccctgcgtgattacggtcgcatccgtgccttcgggagtgaccatcagaatgttgaaaattcccgattcccgcgaactgtaaacaatccggtctccgttggtggtcttggcccaatccgggctcatgttgtaactgccaaaataactaacccgctgcgggtcgctgccgtccgatttcatccgccagatttgcgggccgcccgaccggtcggagacaaatgcgatctgatcgccgctcggcgaccaggcggggaaaatttcgatgccgtgggcgacggtcaggcgtttgggtttgccgccgttcgcgctgaccgtataaatctccatgttgccgtcgtaggaaatcgccatggcgatgcggctgcccgacggcgaaaaacgcgtggtgatcagaattcccttgatcttgagcagtagtttcggttcgccgccggtggtcgggaccacgtacagcgagttgtacatatcggagacattggcgctgttttcccaggcgaaggcgatctggctgccgtcgggcgaccaggtcgggttcttggccttgccgccgaggtacgtcaaccgtttctgattgcgtccgtcaacgtcgatgacgaaaatttcctgcgccccgccgaccgtggaggcataggcgaccttggtgccgaagatgcctttttcgcgggtcagttcgtaaaccacgtcgttgccgaatttgtgcaccatcaggtaccagtcgtcgggtttgccgctgtattccttgccgacgagcattttctgcgtcggcacgtggtagagccggaactggacggtcaatttgccgccgtcgagcgcgaagttgcctttgatgaggtattcggcgccgatcaccttccagtcgttgaaatcgaactcgccgggtttcacgcccgccgcgtgggggttttccacgtacatcgagcgatcgatcacgtcgaagcggccggtcatccgcatgtcctcggcgatcaggtccgacaatttgagcgacaagcccaggttgtcggcctcggcgccggtatttttaaacagcggtaccgcgatcggatccttgcgtaccatcggcatgccgctcgtggtgccgtagatatcgatcaccaggccgtcgccgctcggcggggtcgggtcttcggtgagacccgccgccagccaggtggcgccgcccaggaggccggtcagcatcaacgtcaaaagaatcgcccaacgtggcctcatcggtccctcgcttgctttatgctagtgttgtttttcgcgcggaataaaacgaatcacgatgcctttgtttcgcaattcgtcggccaacagcgacggcggcggcggcaacggactggctttgacgatcgcggaggaacaactccggtcgagcggctcgaagcccgacggtttcgccatttcctgtttgagcaccgaaccgttggccgccacgtaaatccgcacatacaccattttttccaacaaatcggccggcaggccgcccggcaacgaccagactgacagaatgcggctgcgcacgttggcgatgtagatgcccagcacgcccgttgtgctggaggtgccctgcccgccgccgcccaccgagtccatcagggcgcgtttttcgcgcagcttctcgatcgcctccaggcgctgccggtctttccacccgagtttattggtctcgacctgccgatgctcggtcagcttgaccgcgtcggcgctctgtttttgctcgcgttccttcatgatatccgacaggcggcgcgggtcggcggtgcgcacggcggcctggtcgcgatccggcgccatcggccccaggccgggccccttcggcaacgagtaggacagatccacttcgatcggttgcggcaggacgcggtcgctgccgaaaattcccggcagaaaaatgaacgccgccaccgccagggcatgcccgaacagcgaaaccgcggtcgcctgggcgatcagccgctcgaaattaatcgcaaacagatagcgcgtcccggcggtcatcctcggttacttcgtctcttccggcggcttggtgaccatcgacagcttcgcggcgccggcctgcttggcgatcgccatggccttcaccacctggccgtaggatacgtcctgatcggcctttaaatataaggttttatcggcccgctgttcgaagatcagcttgagctgctcacccagcgtgtccagggatacctgggattttttcaccgacatgccgccgcttttgtcgatggtcagcacgaccggctcttcctcgtgctgttcgagcggccccgccgaaacctggggcacgttgaccgacatgccctcctgcaacatcggcgccgtgaccatgaagatgatcaacagcaccagcatgacgtcgacgaggggcgtcacgttgatatcgctcatcaggcggcggtcgccgcctcccgcactcatcgccacggattatttctcccgcggagcggcctgcaaaatgtaccgctccaggatgttcatgaactcgctcgaaaaactgttcaggctgctttccagcgaccgcagtttcgcggcgaagtggttgtaaaacacgaccgccggaatcgccgcgaacaagccgaccgccgtggccaccaaggcttcggaaatgccgggggccacgaccgcgagcgaggcctggcccatggcgccgatgctgtgaaagctgtccatgatgccccaaacggtgccgaacaaaccgatgaacggcgcggtattgccgaccgtggccaggaacgggatggctttttccagcttggccatctggtcggtcatcgagcgctgcaaggcgcggctgacgttcatcttggcggccgaaagccgttcgcgatcgagatcggaatgcacgtcggcggcggtgaactggtcggcgccggcccggaagcgcttcaattcctcgtagccggcggcaaaaacctcggccaacggcgcgtacatgagctgcttgctgtggcggtagatttcctcgagttggcgcgtttcccagaaaatcttcatgaagatatcgctctctttgtgagcgcggcgcagcagcagccacttcaggaagatgatggcccaactggtcacggaaaacacgaccaaaaagcccaggacgactttcaccaccggcccggcgtctttgacgaggtccaggacgttggcaccttgagcgttggtcgcggcgatgacggataggatttcagcagtcaaaatcgattttctcctgtttgccccacggttctcgaattggcgacaaacgtatttcacaaagcccgagaagtcaagatttcaggcgtatttatttatatactacgcccctttgcgaaagtcgcctctacttagaaaccgtcgcgggaaaaaagttgcgccgagattggcgcgcccgatgttttgagttagagtaccccacgggtcggcagtcaggtagggaggtttgatgaaccgccgagtgattgttgtcgccgctttgatcgcggcgctgatgggagtgttgttcgctttttccacctgcggcaaagacgacgacgacgacaaagacgacgggaaggacggcaagactccgccgcaattgtacgacatgaccatctacggttggggctttggggattcgctctaccactatcaagccggccaatggaacgttgacgcggcgacgcccttcagcaaccgcatcatcggcagcgcctttttcaaccgcgcctacggtttcgtctggggccagcgggtcgtttaccgctacaatgcgggcgcctggcagaacctgaccccgccgggaatgccgaccgccgacatcgtcgacggcgttttcctgcccgacggctccttctgggtagccgccaacgatgcgctttataacggctatctggtctgcttcctgctcgacggcagcgccaacatcatcccggtgcaaggcgccatcacctccaatccgatgtatctggtggccctgttccccgtgccggaagatcaggcgatccacctggtcggcgtcgtggacggctggtaccagcacttgcggtgggacggctccacggtgacccccgacccgatcttccaggttcaatacatcgaggtcggcgccgacgacgacgacgataccgccgccgacgacgacacctcggccgacgatgacgacgacgacgacgattcctcgccggtgtacgtccccgccgcccagatcctcgacgcggcccaagccgccgacggctccatttgggcggccggctacagcatggtcgagggcgaaaagcgcggcaccatgtggcaccgcggagccgaggccacggaatgggcgtcgttcctgttgacgccgaccaccggctgcaaaaccaaccgcgtgcggcgcttgtacttcaccgccgacggcgccgggctcgccatcgccgattgcctctactcgcagatttacgaacaggtcgccggcgaccagtgggccgaagcgtcgctgcccggcgtcaagggcgacaagtacgtgctcgaggacatcagtctggtcgacaaggatcgcggctgggccgtgggctacacgtccgacttcaacgaacccctgttcctgatgcgcagcaccgaaggttggcaactggcccggttcatcgagggcgaccagggcgatcacctttacgccgtgaccatgttcgatatccccgaccccggccttccttccgatgacgactcggcgaccgacgacgacaccagcccggccgacgacgatgccgccgacgacgacacttcgctgcccccggccgacgacgacgccgcggaataaattcgccaccccttaatctccccgggcggccgcttccggccgcccggtttttttaaaaccttttcagccaaacggcattcgacttttcaagtttttcactcttaagccctgttgacgacacatttcatcccggattatctttgccggggttaattgggtgtcataactttcctatcaacttggttacgcgacattgggtattggaaagcatgccatcatggaacagtcgcgctgcccatgatcgcgacaagcatcatgaacacacacgattgccacaataaaaaaatgccgatcagggctcgccgccataaatggattcgactacttggcgtagtcgaataaatccgggcggtggcctgcgccgcaacggaaaacaagcaaaaacacttatcggttcaaggatgggagtatggtcatgaactataacttgattgaagaaaagtggatcccggtactatggactaatggtaaagtaagccgcgtcggcatcaaggacgcgcttacccaagccggcaacattcgacagatcgcggcaagcaatccaatggaccggttcgcgattttccgctttctgttggccactctgtattggtgcaaggggaatccttcagaagaaattctccatgatcttactgactcctcttttcctgcatcttggtttgccaaaatggatgcggaaaaagaatgtttcaatctctttggcgagactaaaaggttctatcaatttctcaatccagggaaagaagggctggaaaaactctcaattaactacctcattcatgagatcccaaccggatctaataaatggcattttagacattccaccgatcatatagatggcttatgtccagcttgttgtgcaatgggattattgcgtttgcctttattcgccacatctggtggtcggggcaaaccaccagcgataaatgccaaacctccaatatatgttgccccaatgggtttttcattagcctcaacccttcgtttttcttggcaagaaacatcaaaattgggaactcccttttgggaaaatccaactggagtatccaaatttttaggtgaagtgccatttctagtcgggctaacttggttaccacgccaaatatggttagatcaaccagaaggcttcgaaagcaaatgtatttcatgcggcagacaagagcatttaataaaacaatgcatttttgccggtatcggaagtacgaaaaccgttgagactagccaagagaaaaaatggacagatccgcatgttctatatgtcaaaacaaacaaaggtgacggttcggaatcgttacatgccagtaatgctcttggagctaatgatgctgctgccggacaatggacaaaaatattatcgggaataattcaaaaacgagaaattcttggtcgagcaagaaacgtttgggttgttggcttttcaacagtacaaaacgataagtacctcgaggcaaaagaatttttcatccccacttcaaacgccgagaatctcttttcggacattcttgaaaaaatcggaaaatggcaaaaagaatatttaaaagttatcaacaaaagcaaccctaaaaaagaagacacagcaaaaaaaagaaaacatagcgaaatcccagccaccatcgccgcgatccggccccacgttgaaaaccaggtctccgccaagatcgacgaattgctcaccggcgatgaaaacgcctgggagcaagccgcgaaggaatattcacccctaatgggggcggtcgccaaatcacttgcgccgggattcacgtccggggccgtccaaaggcaacgacaaattgctatcgtcaaaccggacatgcgggaaaaagccgttgccgaaaataaaccagccaaaaagaaaggggatgagcaatgagcgccacggcacaatacattgaaagcctcactaatcttcaatcaggcgctcttggtttgctgcggactcaggcgggaaaagggctcgatgaaacggtcgacggcttcgacctgttcgcgggcctctggtggccgttgaggcaaaaaaatcaaaaggcaccgcgacgtgaggtggcttggctgatcgccaagctctacgccttttgtccactcgaaaatacacccggaaaaacgctggccggccaattacgcctctgccagccacataaagatccggggaaaattcgcttcgctcagcggttcgatcgaatgctgcttttaccgctgggaaaaatcgagcccgcgcttcaatgggctttgcatctaatcgccgacaacgacggaaagctggattgggtcaagctgaccgacgatctttcattctggcaaaaggagtccactcgactgcagtgggcaaatcaatttctcgcaacagaggaaagggaataatcatgttgatcgaaattcatgctattcaaaatcacagcccggccaatttgaaccgcgacgatctaggcgcgcccaaaacctgttacttcggcggctttctgcgctcccgcatttccagccaatgcatcaagcgaagcattcggatgagtgacgagttcaaatctctttgcggcggagtacgcaccagacaattggcgaaactgattgccgaacaggtcaccggcaaggctgacgtgaaaaagcgcgcgaccaaaattctcaaggaatgcggtatcaacccaaaggacgaggaccgcagtgatatgttggtctatacgactcacgaagccatccgcgaaatggccgccctgctgcaaaatgaagaagaaaaatccgatgaagccctggcgaatgaattcggaatgctgatttcccaatgcgtcgccgtaccggatatggcgctaggcggtcgcatgttggaaaccggggtcctcaaggacacaacggtggaagcggcgctgcaagtggcgcatgccatttccacccacgaggctcgtccggaggtcgattattacgtcgccgccgacgacatccccggcgacgacacgggtgcgggatacgtggatgaagccatgttcgcctcggcgtgcttctataagtacttctcgattaattgggaaacgttggttcggaacctacacggcaatcgggaattggccgcgcataccgtcggcgcctttattcgcggcgcggcaatggtcaatcccaccgggaaacaaaacagctttgccgcgcacaatccgccggacgggatattggtcgagattaaaaattccccgatcagttacgccaatgccttcgccaagccggccatgcagggagaacgtgaccttgttgcgcaaagcatcgcccaactcggtcaatatgtgcatgatctcgatgaaggattcggcaagccacagcagcgcttctggttctcccccaacctgcgccattccttgagcttcattcatgaaaagaaagaaaatccgctcaccgacgacaacttcaaatcgcttgacgccctgatccccgccgtgctcgcagccgtcggttttgattggcaggcggtgcaaagcgtcgtcgcgattcaggtgtgaccatggacgccaacaccttgttgcttcgtcttgccggtcccctccaggcatggggaaatcaggagtcaaaattcgtcgttcgccgaacggcggaagcgccgacgaaatccggcgtgatcggcttgctttgtgcggccttgggcatttcacggtcggaaaccgccagcgaatggttgccaaaattgcgcgccctgcgaatgggcgtgcgcctcgatattccgggcgtgcgctggtgggattaccacaccgtcggcgccggaatgaacatgcggatcgccgaaagcgagggtaaaaccaagcccggtgctctgctcacccgacgtgagtatctgtgtgatgcttcctttctcgtggcgttgcaaggtgaaccgaaactcatcgccgatctggcggcggccgtgaagaatccgaaatggacgctattcctcggccgaaaagcttgcccgccgtcgcgtccgatcatcgaagatctcccgggcgcattccccgatttgctgacggctctgtgttccgtcccctggcaaaaaagactgaacaacgaccaactgccggaaaggattgattgcctgctcgattgggaaccgacgccggatcagccgatcgcgcccgctgacgcgctggtctggtatgatgttccgttgaccttcgaccccccggcccatgaaccgcgattcgtcattcgccgttactttcggtttggcgaaaacggcgaccttcgcctcgcggaaaaagccgcgcagctttcgactccgcctccgccgcgcccgcgcgcggactatcggaactccgaatatcgccacattcgcgcggctcgtctcgatgccgacaagggtctttgcgtattctgcaaatcgcccgccacgaccgtacaacacatcacctaccgccacgccggcggcaatgagaacatcgaggaattgcgttcgctttgccgcctctgccatgacgcggtgaccatgatcgaatacggattgggcatgggcttggatcgcatcaatccggaggacccgcaatggcgcgaaccaatcatccggaagcggaaagaaattattaatttccgttcgctggaaacccgccgccgtcgtctggccgccgaggaggtggaatgaacatgtacttgtcataccttttgatcgataccggcgataaccccgaccggccgcgcccggggcgcttgtggatacggaaccgctaccacgttcatcagcgactctgtatggcgtttccgaaccggaaccgaaaagccgacgacccggatttcattcaaccctttcaaccggcggatttcggtgtcgaccaggtgcacgttcctcgccaaccggaccaggggttcctctttcgcatcgatcccctgcccggcggccgcgtggcgattctcgttcagtcggcgacgaaaccggattgggactacgcttttcacaatgccggatttcttttggccgcgccgccggaaataaaatcgttcgccgtcgatttccggccgcgacaacgtttgcgctttcgcttgttggcgaatccaacccgcaagatcgatacgaaaaccgggccgggcgggaaacggcgtagcggcagacgcgttcccctggccaagggcgaactggcggcgtggctgatacgaaaagccgccacggccggcttcaacgtcgattcgaacgccctcgccattcagccgagcttcgtctatttcagaaaaaccgaaaagcacctgccggaaaattccgctgacgagcagcgccccggcgggcaaaaactccgcgccgtgctttacgagggaatcatcgaggttgccgaccccgaaaaattgcgggccgcgctggtcgccggcatcggcagcgccaagggattcgggtttggcctgctcagcgtggcggcgtgtcgttagactttaacggcgaaacgttccttgaaaaccgaagaagaccgaattttccaacaatgggagggtcacatgatcgtgaaggatctgcatgagttgcccaaggttcgcgacggtttgagctttctttacgtggaacacgcgcgcctcgaccgcgagggcggcgccgtcgcgatttacgacgaaaacggcgtgacgccggcgcccgccgccgccctggctttgttgctgctgggacccggcaccgccatcacgcacgaggccgtgaaaaccctcgccgaaaacggctgtacgattctttggtgcggcgagggcgcggtgcgatgctatgcgcaaggaatgggcgaaacccgcaaggccgacaagttgctgcgacaggccgagctttacgccgatcccacgcttcggttgcaagtggcgatgcggatgtaccggatgcgcttcgtgcagccgttgccggcggatttgaccatcgaacaattgcggggaatggaaggcgtgcgcgtccggcaggcttatgcggaagcggcgcaacgctacggcgtaatctggagcggccgcaattaccaacgcggcgactggacggccgcggacccggtcaaccgcgccctgtcggcggccaattcctgcctgtacggtttgtgccacgcggcgattgtctcggccggctattccccggcgatcggcttcatccacaccggcaaacaactttcgttcgtttacgacatcgccgacctttacaaaatggacgtcagcgtgccgatcgcttttcgcgtcgccggcgaaggggtcgccgaattggagcgcgaggtccgccgcctctgccgtgacgcttttcgcgaagtgcggctgctcgaccgcgtggttcccgacctcgcccgcgcgctcgacatcgacgaatcgctcgacgaaacgacggaaaagaattttgacgccgacgccgccgaaccgggcggtttgtgggatccgtcggcgggcacgctggaaggaggtatgaattatggtggtgatgattctggagaacaccccgccgaacctgcggggccaactgacgcgctggatgatcgaaccgcgggcgggagtgttcgtcggaaagatgagcgcgatggttcgtgaccggctctgggaaaaagtgatccgccacaaacgcaccggcggatgcattcagatttatccggccgccaacgaacaaggattctcgatccgcgcggaaggcgataccagccgaacgctggtcgacttcgaaggacttacactggttgcgcgacgaaaaactctttagtttcgaagagtgctccccccgcacggggggctgaaccgttctctatcggcccaatgacgcgcatttcgtcgtgctccccccgcacggggggctgaaccgcggt

SEQ ID NO: 9 - DNA, 15358 bp, unidentified
  misc_feature 1..15358: note = JACQQK010000158_organized
  source 1..15358: mol_type = other DNA
agctccaagagggggaaatccaaaagaccgggaaggaaaaggccgcatagcctgtatcttcagtcgccctacgggctccttccgattcaggctatccaacatcgaggcaaacgcttttacagaaattttgttgcactactatcattggcggcgaccccgccctttccgcgcgtctccttaaactggccaatagttcgttttacgggttcccttcgaggatcgagaccatcacccacgccataaccgtcatcggcatgtcgcagttgcgcgacctggtgctggccaccaccattgtgagtcagttcaagggcctgcccgagagcacggtcaacatggagtctttctggtatcacagcatttgcgtggggttggccgcgcggatcatcgccatttatcacagggaacccaacgccgagcgttattatgtgttgggcatgttgcatgacctggggcatctcgtcatgtttctgaacatgccggacgagatgcagaggattttcgagcggtgcaaggaaaacaggtcgttggtgtaccagggcgagcgcgaggcgttggggttcgatcatggcgacgtcggcggagcgttgttgcgggcctgggacctacccgcgcggctggaggaaccggtggcctgccatcacaaaccttccgccgcggcgcgctattccatggaggcggccatcgtgcatctggcggacatcatttcccatactctggagatcgggcacagcgccgaggattgcgtcgccccattggacacaagggcttgggaaacgatcaatattcccatcagtcagttgtcttccatcgtcaatcaaatcgatcgtcagttgcccgacgctgtccagatgttcatgcctgaaaaatagccatggccacgccgaacgaagacgaaaaccaggaacaaaagatgcgcgtgcgcatcgattttctggaaaaattggcccagaagcatctctttgggctggatttgctggcttctcttggcgaattgcaacacggcgccagtctgcgcaaggacccgcagggcatttttaatatcgcccgccagcacctgaagtttttaacggcgttcgatttcatggctttcttcgtcgtggacgagatggattacgagtttgtcctccaggatttcgagccggagggcgaacgtcagcgcgtccaggaggaagccgaccggcaaatagaaaacgggacattctcctgggccctgaaccagaaccggcccgtcatcgtcaggtcctcgtcgtccggcaagtcgctggtcctgcacgttctcgccaccaagtcccgcgtccgcggcatgtttgtcgggcttgtccccaaaagcgagatcgaactgaacgacgcgatcacctatcccttgtcggtgattttgcagagcaccgcgaatgccctggaaagcgccgccctgtacaagctggctttcgagcaaaatcagaacctcgaggaaatggtccgcaagcgcacccaggcccttgaggaacagacggtccagttgaaggaggaaatcgcctaccggaagctggtcgaggaaggactggtggtggccaaggaggaagccgaatccgccgccaaggcgaagagcgagttcctcgccaacatgagccacgagatccgcacccccatgaacgccattctcggttactgcgaaatcctgcaatacgagtgcaagaaactggataaccttgggctcatggacgacctgaaggggatcgagtccgccggaaggcacctgctgagcctgatcaacgatattctggacttctcgaaaatccaggccggcatgatggagttgcatttggaggtctttgacatccaggccatggtcgccgacgtggcgaataccatcaggcatttggcggacaagaataaaaattcccttgagatattttactccgaacccataggtctgatgcagagcgacatgactcgcgtccggcaggcgctgttcaatttgctcggcaatgcctgcaagttcacggaaaacggccgtatctccctgcgggtttcgcgggaaacctggcaagagacggaatggatccgtttcgtcgtctccgacaccgggatcggcatcgagtccgacaagttggaaagcattttccacgagttcacccaggccgattcctccaccactcgcaggtatggggggacgggtctcggattgtccatcagccgtcgtttgtgtttgatgatgggcggggacctccgcgttcaaagcgaggtgggcaagggatcgtcctttacgttcgtcattcccgcggaagcgaaacccggctccagaatttccatcgacaaggactctcctctgttcaggcccgatttcgtgggggacaagcgccgggaagccgccgttgtcgcgcccctgaaggcggagccgcctcccggggaaaagccggccgtcgccgccagaaaatcccggcagaatagcatcctgatcatagacgacgacggggttacccgcgatctgatcttgcggttcctcaagaaggaaggcttggaggcggttaccgcgtccaacggcaggatcgggttgcaagtggcgagggagtcgctcccctctcttatcatcctcgatatgatgatgccggaaatggatgggtggaccgttctgcgagagttgaaaaccgatccggctttggcccacatcccggtcgtcatcctctccatggttgacgaaagagaaaaggggttgagcgaaggggcgtccgattatctgaccaaacctctggattgggagagttttctttccgtattgaacaaataccgcgtaccccatctttcttcccccattatgctggtggaggacgatccgagaaacgccgatatgatgattcgagtcttgaataaatcgggctgggccataagccatgcggcgaatggacatgaggcgttggattgcatgcggtcaagcgcccccaaattgatcttgctcgatctcaaaatgccgatcatggacgggtttgagtttttgagccaggtccgcagggccgaggaatggcgttcgattcccattctggtcctcacttcgaaagagttgaaccaggaagaacggttgcgcctgtcggaatattccgacggcgtcctgttgaaaggcaattacagcaaggaagacctggtcaaccagattcggagatttgccggatacttcattcccaagaagagcggctcgtaaaagcaaggcgcgacggctccctttgagagttattcttgagaaggtttggagggtccgcccggctttgtcccgggttccagcctgctgaagaaaatcgacttcgggcttatccgcaacgcgtatttccccttgtccttgccggcttgggcgtttttggtcaacgcgaatttccattcggcgaccccgcccggcgcgagacggtcgattttcaacatcattcctccctggttggggttgttgaccaaaacaaaattggaggcgttgccgtcgttcccctcctggtaccggtacacccagccgaaaatattggagatggaccgcgcggagttgttttttattttaaagacgatggagctcgcatccggggccagggtgtaatccggagagtatgaaacgaggcccagattgatgaagttctccttttgggcttgtttttcctgcgccgacccctcgcccgggaaaagggcgccggataaatgggccaggaaaaaaagcgcaatccatgttttccgccacgcggttttcatgggcctattttatctctttgtctctcattgtcaaaacgcattgctcgcggtttccggggaaaacggaatggctccggatggggccggtatttacaatcgcgcttgaaagaggtatgttacagttaaggtgtctttgattctgcgcgggtatttcgccggcgcgggaaccatcgggcatttcaaggaatccgggcaatgggcgataagaacgattcccccgcggtttacaaggtcctcttcgtggacgacgaggcaaaggttctcgatattgcccgcaagtcgttgagcggcccgaactacactctgttgacctccgcgggttcgctggaagctttggaaacggtgtcgaaccgggggcctatcgccgtcgtcctctccgataaccgcatgcccgcgatgaccggcacggaattcctggaaaagatcaagtcgatctccccgcataccgtcaggatcctgaccaccgcctatttggacccacaggtcatggaggacatggtcaataagggcgaggttttcaggttcctcaagaaacccttggacttgcaacaggcaaaccaggccatcctggatgggttgaagcaatacaaaaaaaatgtcgaggagtcggagaaacggtctctcctgaacaagctttccgcccgtcacatcaaactgcggtccaggtctgaggaactttcctcgaaagtgtcccgcctggaaaaatgggtcaagatattgtcgctcgcgatcgtcctcatggtgttttctttcgcgggctacgaagtcgtcgtcaactattggaagccggaaaaacccgccggggagccggggacggtcaacggctggataacgcgtcccgacgggaccgctctggatatcaggaacaatctgatgtggatgacccgcgacttccgcggtattgaaaagagacagcccaagaactggacggaggccatggaatgggccgataaaatgaacaaggaaaaattcgccggtcacggcgattggcgggtccccacgatcgccgagtacggcgggacctacgacgccgacaggacccggctcgcctttgacgggaaaaaggattaccccgtggggtatccgaaggcgttcgaggatggcggcggttatgggttctggtccagggaacaggcgggcatggacaaggcgaaatattatttcttcatcggcggatacgagaagaccgagagcgttgaatacgacaaccccaccatgagcgtccgtctcgcgcgttctccatgacctttttcacggtcgcaatgccgcgaggaacagccagtaaaccagcgcgtcaatcgccgtgagggggatccccacgcgggcgaactcgagaaaagtcagcgtctcatgggaccgtttttccgcgttctggatgatgatgacgttgctcgccgcccccaggatgaacaggttgcccgcgatcgtgctccccgccgccagcgccatcaactgtccggtcgcgtccccggaaggagtcagcatcggcagatacagcgcgaccaacggcacgttggatatcaactggctgagcaggacgccgatggccaaaaccgccggtatggaagccatgccccccgttttgccggcgatccgcgattgaaagaaacccgagtcccagacgctttgcatcagcacgaacatcgccgcgaaaaagaccagcgtgtgccagtccaggtttttcaagatgacccgccgtttccggctgaataccaggatcggcagggccgcccaaaccgcgatataagtcaggggaaatcgaacgtcgatgcccagatgggcgatgaccagtttggtcgccgccaggacggcgatcagccccagggaaactcgcgacaggaccgcgagttggtggtcccggatcggctcctgcgagtggacgaggggaacggagtgaaactgctccttgtaaaagaatttcagcaggagataggccgacagcagactcaaaagggtaggggccagaagaaatttcgcgaatgtcgcgaacgggttggcgatgccgccgcgcagggcgatcagcaggttctgcgggttgcctatcggactcgccacgctccccgtcgttatcgcgaaggccagggccagtagcaacagtttcggcgagacgccgtgtttgcgcgccagcaggaggacgacggggacgccgatgatcgccagcgtgtcgttcatcaaaaaggcggaggctatccccatgccgaagaggatgaacaggaccatctggtccgtcgttttggccttttcgaagaacttgtaggagatatgcgcgaggtacccgctcttttcgagcgcgtgtcccaccacgaacatcccggagaggaacaacatgacgtccaggtcgatcgcccggagcgccgcgggcggagaaatccgccccgtcaacaggaccgcgcccgcgcccgccgtcatgatctgccagatcctcaatttcgcccggaagacttgccggaaggcgatcaggaggaatacgatcgacaggacgataatcgggatgttcatcgagtcaggaggaagggtaagcgttcgctaaacggccgcatgaacaaaagtctgttacgaaggaaaatcccctgggtaaaatcccctgggtaaaatcccctgggtaaaatcccctgggtaaaatcccctgggtaaaatccccctcagtccccctttgttaaagggggatggattctctcccccttgtaaagggggccggggggatttgtatttgcttcccctgttataacctggcaacccggtcagcgtccgggaggaccgcccgggccgccccggtcgtcgcgctcgccccggtctctccggtcccggcgttcctcgcgttcgcgcattttttccttacaggcgccggaaaggctgtccttgtgttcgcgcatgcagtcgcgcatcctgccgctccccggctcgacgtcgccgcacaaccgtttgatatcgtcatggcagggaccctcgcgtccgccgcgtccgcccggaccgccgcggtcgtcgcggctttcgcgtccaaaacgttccccgcggtcgcccctgtcgttatcatcgtcccggtcccggcgctcctcgcgttcgcgcatcatggccttgcaggcgtcggacacgctgtcccggtgttcgcgcatgcagtcgcgtattctgccgcttccggggtcgatgccgccgcacagccgtttgacgtcgtcatggcacgggccttcgtgccgttcgccgcgtccgccgggtcctcccggaccgccgcggtcgtcgcggtcctcgcccaaggcaaggtttggagaggaaacgagcagaatcaatccggcaacgagcaggggaagaattctcatacatcaagtcctccaaatagagtaagagacggcggaaaacgggttaaccaaggggagcgagaaccccccttgcaaaatccccccggcccccttcgacgaagggggaaaaaatctaccccccttgcaaagggggagggggggattcgcgctactcgccgtcatcccggcgacagccgggatccagaaggggttgcgccggtcaatgatggtggccgccctcgccatgggcgtgaccgtgcgacagttcttccttcgtggcgtcgcggaccgagacgacctcgacgtcgaagttcaaagtctgaccggcgagcggatggttgccgtcgaggaaaacgtcgtcgccctcgacttcctggacgacgaagtgctgagtatgaccgccccccgtctccgccgagaactgctggcccggaaagattttcagttcggcggggaagttcgaccggctggccttgatcttcagttccgggacgacctcgccgtaacccgccaccggggagaccgtgactttcctcttgtccccgatcctcagcccctccagttccttttccaaacccggcacgatttgcccgttgccattcagatacgatatcggctcgccggcgacggcgcggtaaagctcctgaacctaggaaatcgtcaaacaataagtcaagccaaccacggaatttttcttaatcatgaataacctcgaaattgaagatttaatatttaatcgagcagtttataacaatcgccagaaaatgggaaggagaatgcgccgcgcctttcttcctcccccttgcaaagggggctggggggattcgcggatttccctgcgcccatccgcggtcatgaccgcccccacccgtatggcccatcctggccgaattcgacctttccaaagcgccgggattttcatatactgtcggtatatttggcatgtcaaaaatttgcatgtcctccgggaggcaatgaatccatgaacgaaaaacgcggaaactataacctgctcgaagagcggtggatccccgtcctgatgaccgatgggaggttctgtcgcttgggcatcaaggacgcgctcacgcaagcgggccggatacggcaaatcgcggccagcaatcccatggatcgcgtggcgatcctgcggttcctgctcgcgttgctgtattggtgcaggggaaacccgcctcaagaagggagtgtcacttccgtcgattcattccccgcggattggttttctaaacttgatgacaataaagattgttttaaccttcttggcgacgggaagcggttttatcaggattgtaccgcacaacggccccaagcggttactcagctaatccaagagataccggcgggcaacaacttctggcacttccgacattcgacagataagaggagtgggctttgccctgcgtgttgcgctatggggttattacggttgccgctattctccgtaagtggcttaagcggcccgaaagagcctaatctgatggccgggatcaatggcgttccaccggtgtatgttgtgccttgggaaaagtcgctttttagtacactcttggctaactgggcgacatgcacgaatatcggcgaaccttcatggatccagccaggcatcggccaaattcaaaataacgatgttcctctgctagctggcttgacgctactgtctaggcgagttttgttgcatgatcccgttaaagaatcggttacatgcatcggctgtggcgagaaaaccgcagttattctaacctgcgaatttcagacggctggcaaacaggagaatgataagtggaatgatccccatgtggtctatttagaccgcaaaccgcgaaagaccttgaggggagctgatttaacttcggcgggcaagttcagaatggatcgtccctggcccgatctgcttgcccgtatagtcgaaacacgcaagccggtatcgctacttattgtcggttttgcaacaaacaaggcaaagaacgttgatgtatgggagcgccttgttaccgtgccgcagaatcacagcaattcgacatccttgtatcagtggcaggataaaagtagaggtttggaggactggttgcgtgaaatcgtacgggggaggagaaaaacaaggaaagacgatgctcgcattgctgtctcagctatcgccgccattcggccccatgtcgaaggcagggtttcctcgaaagtgggcgagttgctggccggaggcgacgatgcgtgggagcgcgcggcaagggaatatcgtccgatgatgtgcgctatcgccagttcgctttctccaggcttcacgacggcggcggtagagcggcgacagcagattgccagcgtaatgcccaacatgcgtcaaaaacagatacaactaaagaaaaatagccggaagaaaggaggggataagtgaccgcaacggaggagtatattgaggagtatattaaggtcctgacacgtctgaagtctggcgaactcgggttgttacgcacgcacgcggggctgggtcttgacgaaacggtggacggattcgatctgttcgcgggtctctggtggccgttgcgccaaaaaaacgagcaggcgcctcgccgcgaagtggcctggctgatcgccaagctttacgccttccgtccaatcccgcattcgccgcgagaaacgcttgccagccaactgcggcgatgcctgcccagcaaggacccggacaaagagcgtttcacgcaaaggtttgacaggatgctggtcctgcccctcgacaaaatcgagcccgccctgcaatgggcgcttgactttgtcgcatcgaacgacttgaagctcgattgggtaaaactgacgggcgatctttccaagtgggagggcgaatccaaacggctcgagtgggcgaagcaatttttggaaaccgacgaaaggagacaatcatgttgatcgagattcacgccattcagaaccacagcccggccaatctgaaccgggacgatctgggagcgcccaagacctgctactttggcggagtcttgcgctcgcgcatctcaagccagtgcatcaagcggagcatccgcgcgagcaaagagttcgagaaactgctcggcggcgttcgcacccggaaattggcggaattgatcgccgacaaggttacaggcaaggatgttaagaaacgcgcggagaatattctcaaagcctgtggatttgagcctaagaagacaaaaaaacagaacgaagaagatgagcaagaaaatgctgacaagagcaagatgcttgtctatacgacccatgaaacgatccgggagatggccgccctcttgcagaacgggggcaaggatgaacaagtggctgaagagttttgcaagctgatcgccgaacgggtggccgttcccgacatggctttgtcgggacgcatgttagagccggacaaatccgctaaaaaattgtgggaaaaacttaacacgaccgtggatgcggcgttgcaagtggcccatgcaatctcgacgcatgaggcccgccctgaggtggattactacgtcgccgccgacgacattcccggcgacgacgcgggtgcgggctatgtgaacgaagccatgttcgcctcggcttgtttctacaagtacttttcaatcaattgggaggttctcgtcgagaatctcaaaggcttcaaaggcgataaagagcatttggccgcccatacagtcggcgcatttctacgcggcgcggcgcgggtcaatcccacgggcaagcagaacagctttgccgcgcataatcctccggatggaatgctgattgaaatcaagggcgcccctgtcagctacgccaacgccttcgccagccccgtgtccggcaagggcaaacgcgatcttatttcgcagagcgtcgcgcagttgggacaatacatttatgacctcgataccggatacggcaagccagaaaagcgattctggttctcgcccaatctccgctatcccttgagcgttgtggaagacaaaaaggacatctttcttgccgaaaataacatcaaatcgctcgatgaattgattcccgcggtaatcaaagccatcggccatgactgggaggaggttaaaaaggtagtcgtagacgcggaggtggtctgatgagcgccaacaccctctttctccgccttgagggtcccttgcaggcgtggggcgctcaggaatcgaagtttgtcgtccggcggacggccgaggccccgaccaaatcgggagcgctcggaatgttgtgcgcggcattgggcgtttcccgtccagaggccgccgaagagtggcttccgaagcttggcgccctgcgcatgggagtccgtgttgacatgccgggcgtccgctggtgggattatcacacggtcggcgccggaatgcagatgcgtattgcggaagacgcggcagagacgaaatccggcgcaatgctcacccggcgcgagtacctgtgcgacgcttcttttctcgttgccctgcagggcgaaccggcgctcatcgccgaattggaggttgcaatgaaagctccccaatggacactctaccttggcagaaagtcctgccctccgtcccgcccgttgctggagcatttccccggcgaatttcccaatttgctttcggcgctcgcatcggttccctggcgtaagcggctcaaggaagacactcccccgcaagccgtggattgcctgctcgactgggaaccgacgcccgatcaagctgaagcgccagacgatgcgcttgtctggtacgacgtgccgctgacattcgagccgccgtcgcatcgaccgcgattcgtcatccgcaaggaattgcgcgtgggtgcgaatggcgaggtcggcatcgcggaaaagcccgcgcagaaccttacgccaccgcctcttcgtcctagggccgactatggaaatactgaatacaagaaacgccggcttgaacggcttgacctcgaccatcacctgtgcgttttctgcaaggcccccatcggcggcaggggcgcgaccgtgcaacacgtcacctatcgccgcgccggaggaaacgaaaccttggacgatctgcgctccctctgccgcctctgccacgatgccgtgacgatgatcgaatacggtttgagcatgggcttggaccgcatcaagcctgaagaaccgcgctggcgcgagacgatcatcgagaagcgcacggaaatcatcaagttccggtcgttggaaacgcggcggcgcaagctcgctgtcgaggaggtggaatagatatgtacatgtcctgtctcttgatcgacgtggggagcaaccccgaccggccccggcccgggcggttgtggttacgcaatgcctaccatgtgcaccagcgtctgtgcatggcgttcccgtcggcgtccagaaaatccgaccaagttcatgtcaagcggggagcgggcgccggttttctcttccgcatcgacccgcaacccggcggcggggtcgcgattctcgtactgtccgccgttaagcccgactgggattacgcctttcgcaacgcctgttatcttctgtcggctcccccggaggtgaagccattcaatacctgcttcgcaaagggacaacgccttcgattccgtctcgcggcaaatccgaccagacgcttgagtaaaaactcgatgggacccgacggcaagccggtaaaggaggcgttgatcggcaagcgcgttcccgtgccgttcggccagcttaagccatggctcgctaaacggggcgaggaggcgggattcgctatcgacatggattccaccgtcgttcagccgtcttacgtctatgtgaacaaaccatggaacggaaaacggtgcgacaaatgcggtgggccaatgaaggaagatggacaggggaaagagatggtctgcgaaaaatgcgaggctcgcgtacaactcttccggcaccgtctgctctccgtccgtttcgacggggtcctcacggttaccgacccggatgagtttcagaagatcttcgccagcggcatcgggtccggcaaggcctttggttttggccttctctccgtggcccccgaccgtccctctttcccggaggaatcgacatgaaaattcaagatctgcatgttctgccgaagttccgcgacgggtggagttacctctacgtcgagcattgccgtatcgaccaggaggcccgggccatcgcgatccatgacgccgcgggcaaggcgcccgtcccctgcgccaacctggccctgttgatgttggggcccggcgtgtcgatcactcatgccgccatctcggtactggcggaccacgggtgtcttgtggcctggtgcggggaacagggcgtccggttctatgctacgggcatgggggagacgcgtagcgcgtcgaacttcctgtatcaggcgcggacgtgggcgtacgaggaactccgcatgcaggttgtgcgcaggctctaccagttgcggttttccgagaaactcgatccggcgttgactctccggcaaatccgcgggatggaagggatgcgcgtcagggagggctattccagggtctcgcgtgagaccggagttccctggtcgggccgaagctatcgccgggacaaatgggacaactccgacccggtgaatcgggcgttatcgaccgccaactcgtgtttgtatggcatttgtcacgccgcgatcgtgtcggcgggtttctctcccgcgcttggatttattcacaccggtaagatgttgtcgttcgtgtacgacgtcgccgatctctacaaaacggaggtcacggttccggcggcgtttcgcgcggcctctgaagggaaagacgggctggagcggcgggtgcgcatggcatgccgcgacgcgtttgtctcgacgcggttactggaaaggatcattccggatattcagaaggcgctggcgcttcccgcgcggggcttggctggcgtcggcgaacggttcgaccaggaactcgccgctccgggcttgttgtgggacccggtggagggacaggtggaaggcggacgcaaccattcgccggaagaggacgaaacggaggccgacgatggtcgttctgattcttgaatgtgttccgccggggttgcggggcgaattgacgcgatggttcctggaaccgaaggctggcgtattcgtgggccgggtatcggccatggtgcgggaccggatctgggagaaggcgtgcgggcaggcaaaggaaggcggttgcgtgatgatccatacgagcgacaccgagcagggattcaagattcgcagttgggggcgcacggcccggacgatagaagacttcgagggactattcctcgtccggatgccctaacgcttgttttttgcagtatcttccccacgcgtgtgggggtgaaccgagtcgccgttgaccacggcgtaaacggcgggcatcttccccacgcgtgtgggggtgaaccgcaacgtcaaaccaatgcggatcaagctcaacgatcttccccacgcgtgtgggggtgaaccgctgttcgacgtttcgccggtagtgttccctgcatcttccccacgcgtgtgggggtgaaccgtgccgttcatcctcgaacaccccctccaaaatatcttccccacgcgtgtgggggtgaaccgtcggggagggccgcgacgacctcctcgacgaaatcttccccacgcgtgtgggggtgaaccgtatcgcccggcgtaccgggccaaaaccgtgagatcttccccacgcgtgtgggggtgaaccgaccgccgcgacggggcaggacgtggccgtggaatcttccccacgcgtgtgggggtgaaccgaaagctgtataatcatgctgtaggaaatgttgatcttccccacgcgtgtgggggtgaaccgaatccttggccacgagtcatattgccagttaaatcttccccacgcatgtgggggtgaaccgtagcgtcgtatgcgacagcccgagaggctgacatcttccccacgcgtgtgggggtgaaccgtgtatcgcctcgacctaaacatttataccgccatcttccccacgcgtgtgggggtgaaccggttagcgcccgaaaggttggctctcgaaaggtatcttccccacgcgtgtgggggtgaaccagggggggattcgcggcgccagcggttgtaatcatcttccccacgcgtgtgggggtgaaccagattggctttccaaaggttggcttccgaaagaatcttccccacgcgtgtgggggtgaaccgatggcgaatggttcaaggagcgtattcaatagatcttccccacgcgtgtgggggtgaaccgggcatatcgatgccggtgtcaaggctgacgctatcttccccacgcgtgtgggggtgaaccgtatgatcaaggatcggtttatttgcgcggagaatcttccccacgcgtgtgggggtgaaccgggcatatcgatgccggg

SEQ ID NO: 10 - DNA, 32759 bp, unidentified
  misc_feature 1..32759: note = MWEE01000013_organized
  source 1..32759: mol_type = other DNA
gtgtcatcccgggcaagcgcacgcttgacccgggatccagtcttttagaaacacccgtggaaaggtggaaatggcggttcagagtagcggcgcttgccatagcgccgtttgggattgccgtccatggggggggatggcaaagtccgtttgccacagagaccggaggtctcttccaagcaggcaactccgttgcctctgccgaacggacttcggcaacccttatattacaccaatccgcgacaatctgcgccttttatccgcgtatatctgcgtgaaccgtggaacccgggatccagtcttttacaggaaagtttgaagtccgagccggcttcaggactggcataagcatgaaacccatattgccggctcgaactctaaacacagccagcatggagttcaagagccttttcagactgaaaaatgccaccatacaagttggctcttggacttcatgcgtaacatattgctggctcgaactctaaacacaaccagaatatatttcatccgcgaggcaaataatgcttgacatagaatctaaaccacacaaaaatagatttgagagggtaaataaagaaaggagcaggaaagatgagcaagagcttagtttttgtgctggcgttcttatgcctggcggctgcgtgcctggggtcggaagcggccgcgcctgaccctccggagccactccgcgcggtggaactgacgcctccgccttcaccggtaagagcgttgccagggcagaagtccgtgcctccctggaacttcagcgtcaatccaaacccgatcataaacaactattacgactacatgatcggcggctacaacaacctgcctctgaacctcgtccccgagagccagggcggaggctatttcctcacctggacggggcgcaacagccccaccagcgaccgcaagtcctattacggctatctgtactcatccggaatgccaatcttcgttaaccagatcgttacccagggagaaggcgagcacggcatgccggcattggtggtggatcccgttgatggaaatcctttctacgcctggacccagaatgtggacacggacccggtgtccgagattgtgctgacctccgatgccttcatagccggaattgccggattgttcaacaccacccagatcgtgatcgacaaccccgtcaccctcaccgcgccggacgggacggagaccacaaacaacgttttctactggcccagcctggccatcggcccctcccctctggcgggcaagcgcaggctctatctgctgggacggcaaaactgcaacgtctatctggcaatggctgatttctcccgcgatgatgtcgaaaatggaatccccctcaactggcagtactccagcgttccgcaactcaacacctgggaactgaatgccgtgccaaacaaaacctatctggccctggaaactgactccggcgggaatgtcatcctggccggatatcacaacagctactcgattgagaatgatatcatcgtcttcatctgcggcaacttcggacagggggaatggacgttccacagcttcaacagcaggatgcccacctggaaccccacctatcccggcagcaacggcggcttctttatagatgatgaggggcaaccctttcaggatcaagacctgcactggaaactgttgaactccggacatctgaacgccaccctggatagcggtggcaggctgcatatcgtggggatttgggccttggccggcggggacggcgtatttttccctcatttccagttcgtgaaagaagctgtttacgacccgacttggaataatttcgagatccgcgacatctttccccaaaaaaattgtgacgaccttttcaatgcctgtttcaccccctgggacatgctgccgccctacggcgaggttgacgaatggatctacgatgaggaatatggttactatccatacacgttcactgactggcccttcccccactgggaccaggcagcacacgaaaacaccatgatgttccactataacaacgtcaagatcacccgcgccaacgactatggccagatggctgtggtctggcaggattccaaacgcgcctttgactggtgcgtgtatcattatcccggctatgaggcctgggaacacggcacggacatctatatcgcctatacaaactatggcggctcggagtggcaggacccgatcgtgctcaacaatgtcgatactcccgaactggcgggcatcaagccgatgtgggtgtatccggctgaccggatgatatacactggcttcaaccagtacggaattaaagagggaaaactgggcctgatgttttacgatgacttcacctggggctcgaattctctcccgccaccctaccaccccgatcctgacggcggccgggtgatgttcatggaactgcatatcggatacctggacgccaacgatcccagccaggcgccagctccggccatcctggcacagaattggcccaaccccttcaatcccaatactaacatcagttttgaactgcctcagcccggccctgcccggctggaaatcttcgacctccgcggaaggctggtaagcaccctactggacaaggagatgggagccggaaaacatagcctgaactgggacggcaccgactccaacggcgcccaactggcaagcggagtctatatctaccgcctcagcacgggaaggacgagcgccagccgcaaaatgatattgctgaagtgacgggcatccgcccttccaagtccgatctttataccgagccgccgcgtgtattgcgcggcggtttgctttccgcagaccaagtatcagcagcggggaactgcgctgaaaaagccgctggtggggctccgtaaaatgggagtggaagccgccgcatggaacaccttgaaagtaacaagagaccgccccaaattgacctttcctgacctaataacccctcaaagcaaatttcttgacaatgatctttggctaaaatataaggttcccatcgagcaaaaggagcatgttatgtacaatcttctttgtgataattgggtaaacgtggtctatctttcgggcaaaccagatagaatatctttagttcaaactcttaaagacgcacactgtctgcaactggcatacagcaatcctatggaccggttcactgtcttcagattcctgcttgctcttggctactggtgtttcgccaacaccaacgtggagcctgaacctgataagccgcttccggtctcctggataccctggctggaagaaaacaaggagtatttcgagctttttggtgatggaaagagatttttccaagccgatcccagttccaggatcagagccattaccgaccttattcatgagataccaacagctcacaacctatgccatttcaaacatgtcaccgactatatcgatgggctctgcgaagcttgctgcatcaagggactgctgagattacctgtctttaccactgtcggtggcagagggataggcgctggaatcaacaacactcctccattttacctgctctggcatgcaaatgatctcgctggcatgctggctcaaaattggcaaccctgggataatatggggattccggcatggttgggttcttttcaaaaagaaagcagggaagtaggcttacttgctggaatgacctggctgccgcgcaaggtgtatctgcatgatcctgtcccaggtcaggcagcatgttgttcctgcggcttaccctccgaagctctcgtctattcctgcagcatagaagtggagccagtgccaaagggacttgagtggaaagaccctcatggtgtttacaccgatcagggaaaaagtctccagtccaaaatcaaactgatgtccaatgatagatacactttcgcggaccgggattggtatagcccgttgttcagctatttgcacgcggaaggaaactccagacaaggaaaactatggttggttggatttgccagcgataaagcaaaaagcatagacatctgggacaagatcatcgagcttgagggaactgatacaaatgatgagctactggctcaattggcaaacagggctactgcccttaatgccatgcgcaagaagcctctcagaggtgatttcaaaaagtcagtgggcactccccagattgctgacattatcccccacgccgaaaacagaattgccatcaatgcgggtaaaatgactgagaacaggggctattcctggcaggatgccgatactgaatacggagaactgctcaccaaggttgcatactctttggagccggctcaaacagtggacgccaggctcaagagaggtaacttcatttcccgcaagccctggccaattataccagaatccaaaacaaaacccgcagagggggaccagaatgaatagaggaaccgttgacttcattgcaagcttggagaacctcaaagaaggcgatttgggcattcttagaaaactcaggggagcccggcttgatgaaaagcttcccggatttgacctgttttccgccctatggtggcctttgcggcaaaagaatcaacgcgctcccaagcgtgaagtggcttggttgatcgccaaactttttgctgaattccgctttgaacagcgcgagggagctacattgcccattcttatgggaggcatctgccggaagttggaacccaaaaaggaattaccccgggtattggcacgctttgatcaactcgcatcgcttgacattatgcagatggaagagccgctgtcggtcatcatgggtattttgcgcaagcaccagcaggtctgcctggactgggtagggcttactgatgttctgtcattttgggagcaggaacctgtcaaaagagaatggtcggattcatttattaaagcatacaagatcaacaaggaggattcagatgttgattgaaattcatatgattcagaatcacagcccggcaaatcttaaccgggacgacttgggcgcccccaagacctgttatttcggtggagtattacgttctcgcatctccagccaatgcatcaagcgcagcatcaggaccagtaacgattttaaggctttgctgggcggggtccgcacccgcagactggctgatctgatccaacaggaagccggtgaaacagaatgctggaaaaaggctcaggagatactcaacaaatgtggcttcaaaaataaggacgacaataccaagatgcttgtgtttatgtccaaagacaagatcaaagaccttgctcgaattgtcctggataattccctggggctcacggaagccgctcagcaagtcgccaatgttatagcccaagcgacccttgctcccgacattgccctctgtggaaggatgcttgagcccaatgacaaggacaaggacaaaaaagtcaaatggtccaatacaactgtcgaggcagccctacaggttgcacatgcgatctccacccacattgccagacctgagatagactattttgtggcggccgatgatgttcccggtgaagacgccggggcgggtcatattggcgaatccatgtttgcctcagcctgtttctacaaatacttctccattgattgggaacagttggtcaagaacctgaaaggggacacgaatctggcagcccacaccgtgggtgcctttctcctcgctgctgccaaaaccaaccccagtggcaaacagaacagcttcgccgctcataactatcccgatggcatccttgttgagttcaagaattcccctattagctacgccaacgcattcgtcaggccagtttccgtggtcaaggaatcggacctggtggaacaaagcatcggccagctttccaactacgtgaacgacatcaggcttggctattatgatgagcaatccccagttatcggcttctggttttctccaaacaatcgctatccccttggctataagcattccaaactggcttccaggaacattggcaacctcaatgagcttgtaggcgctgtgttggattatattggcggcttcaagtgggaagaagttcagaaaagcaaggcttatattggcggctaacatgagtgcgccacccaacaccttattcttgcgcttggagggggctttgcagtcctggggcagcaacgaggctaagtttgctttgcgcagaactgccgacgctcccacaaaatcaggtgtcctggggctgctttgtgcggccatgggaattgggcgcgcggaggctgctgattcatggctcccaaaactcgccaacctgcgcatgggcgtgcggatagaccgccccggcatacgctggtgggacttccatactgttggagcgggacaaagaatgcgcatggcagaattgaaggcacccaaaaaaccttccatggttggagctgccctggctgaaaccctgacaccaagcaaagtaaaaaccagggcagagaccttgctctcacgccgcgaatatcttgccgacgccagttttctggtcgccttgcagggtgagccggaacttgtggcaaagctctccgccgctctggccaagccggtctgggctatttatctggggcgcaaatcctgtcccccttcacgtcccgtgtgcgagcatcctccgggcttttacaacactttggaggaggcgctgtccgcggttcccctgcaaaagcgctggcacaatgaacccctgccgcaaatactgccttgcgtcatggactggattcccggctatgacggagaacatgcgcctgatgacgcggagattcattacgatctgccggtttcttttcagccgccccgccatttgccgcgcttcgtgatcaggcgtgagctggttgtgggagaggatgtgcaagtgagcagggaaaccggaaccagcgtttggaggcccaaaggaacccgggcggattataacaattccgagtataagaaggtcagggcggaacggcttgtgatggaccatgccgcgtgcatggtatgcaaagctcccgccacgactgttcaacacgtgaattaccgcagggcggggggcaaggaaataccggaagacctgcgggcgctgtgccgcctctgccacgacgcctgcaccatgctggaatacggttccggcatgaccaccaaccgcatcgatccctgcgaccccatctggcgggagcgaatactggcaaagcgcaaagaaatagtcgagttccgttcgcgcgggcagcgtttccgcaaaatgaagccagaggaggaaaacggatgatctacttgtccaggctattgatcgatactggaggcaatccagaccgtccccgccccggcagaaagtggctggacaatatctataacgtccaccgccgcctttccatggcgttcccttcaggtttgcggcgggagcaggatccgcatttcctgaagcccttttcgccaaacgacttccaaaaaactcccttccttttccgggtcgataacaacatcgacggaaacgataaaagggcgattattatcgtccagtccgttcttgagccggattgggattattgcttccaaaacgcgcttgatttcctggccgcgccgccggaaacaaaggagtataatccagagtttaaagcgggacaactgctgcggttcaggctgcgggtaaacgccagcgtacgccgtcatattcctgaaatggtgcaacaggacggtcaaaccatagaaactggcaaaatcctgcacaaaagggtttctcttacctgggatgcttcttccaccccggatcaggcccttgccgactggctggctgccaaaagcccaaagctgggcttcacccttcagcgctgtgagcttcttcaactgggttgggtttatggctccaagccagaaccaaaaaatgtcaaagtaaaggaacaggggcagggctactggcgtgaacacaaatacaatcctctgcgcttccgtgcggctctcctggagggagttctcgaggtggatgacccgaaactgttcctgaaaacgcttagcagcggcattggaaaagcgaaaagcttcggctttggcctgctgtccgttctgccaatcaggaatgatggataaagacctgcactctattccccggatcagagaccggcttagctatctctatgtggagcatgccaaaattgaccagaacgggcaagccatcgccattcacgacgctctgggtgaaaccccggttccttgtgccggcttggctatgctgatgctgggaccgggcataaccatcacacatgcggcaatactttcactggctgcctgtggctgtctcgtagcctgggtgggagaacatggcgtcaggttctatgccgcaggcacaggtacaacccgcagcgctgtcaatcttatgaagcaggcaaaatactgggcagacccagttttgcatctacgggttgtacgcgccatgtaccagttaaggtttcaggatgagctttctccagatttaactttgaaccagatcaggggcaaggaaggtgcccgggtgcgcgatacatatttccaactctcccaaaagtatggtgtgcccatgcagggcagaaactatcaaagagacaactggaacaaagcagaccccataaaccgggctttatctgtggcaaactcatgcctctacggcatatcacacgctgccatcgtagcggcaggattctctcccggattgggctttgtccacactggcaagcaactgtcgtttgtctatgatatcgcggatctatacaaaatggagatggctgctcccgccgcattccgggcgaccgctgaagggatcggcgaactcgaccgcagagtcaggcttgcctgccgtgatgaattccacaggcagaatctactcaaacgcattgtcgctgacattagttctctcttcggtgatattatgccagaggatagcccggatgactgttttgccgaagaaccagccttgcctggaggactctggtctccagatggagaagtgtccccagggggcatatcttacgggcaggatgatctgccaaaggaagattgatggtggttttggttttggaaaacgtgccgaactccttgcggggtcagatcacacgttggctgctggaagtaaaggcgggggtgtttgtgggtaaagtctcagcaatggttagagacaaactgtgggacaaggttagaagcaactgcagcggtgggacttgcctgatgatctggagtagccagaccgaacagggttttgatatcgaattctggggtgccgcatcaagagtcgttacggaatgggagggtatcaaactaatgaccaaacatccttgaactaaggcttgaaggagctcaatgacctgttagctctctattgatcaagtgtattccccacgcatgtgggggtgaaccggcgtcaatacggacatcaattctaatgcgctcgtattccccacgcatgtgggggtgaaccgccgctcgcaattatgatggcggactgttttgggtattccccacgcatgtgggggtgaaccgtaatacttgacaagcagcttgtgcgctatgccgtattccccacgcatgtgggggtgaaccgaccaaattgctgcttcaaacgacaatcgtgttgtattccccacgcatgtgggggtgaaccggttcaacaggcaaacgtattgtaaagtgttaggtattccccacgcatgtgggggtgaaccggcatcattgaaaccgccatcccgcagaaaaatgtattccccacgcatgtgggggtgaaccgcctgcatagtcaagacgtgggtggcgtagcccgtattccccacgcatgtgggggtgaaccgagggtaaacggaagcggatgctgaacgaatacgtattccccacgcatgtaggggtgaaccggtgccgcggtgtccctgggtgatgccacgggcgtattccccatgcatgtgggggtgaaccgaagccataggcgtcgtagcgctctataattgagtattccccatgcatgtgggggtgaaccgctggtctttccgacgctgatggtgggacaatcgtattccccatgcatgtgggggtgaaccgagcccggagccgtttacgcgggcgaactggtcgtattccccatgcatgtgggggtgaaccgaccttgtttggtcagacttagcattagctcatgtattccccatgcatgtgggggtgaaccgacttgacagaaaaacaagcctacggagaaattgtattccccacgtatgtgggggtgaaccgtgctcgatatggaacaggtcgccgtgcatcgagtattccccacgcatgtgggggtgaaccgacgacctacgcgcagcggatggcgttcagcaggtattccccacgcttgtgggggtgttgcgatttcctcgcgcgcgggcgactctcatttgcatctggcgctcagaccttggtcttcctggggtcaaccttaacctttatatggatgacctaaggatgatggcaccaaagccagtagcagcaagggagagcggtatgaagcaatatgaggattatcgcaggaattatcttgagggaacaagcatccagtctgggtagtttgttggtattttatcgtttgtctaaagtggtggttttgtgattgacctgaaatcggtgtgacggggttttatggttttttgaagttcttttttgtatcgtgatgtaaaattttcaccctctttctatctgtgtaatttgtggttttattaaaagactggatcccgggtcaagcaagcttgcccaggatgacgcagtatatagcttgggggtggtttgtttcaccattccacctttctacttctaacaattcgtgttagtttgtgtaatttaagggctgccgaagtctgttcggcagaggcaacagagttgcctgtttgggagagacctccggtctctgtggcaaacggattttgccacccctggacactggatcacgggtcaagccacccgacagcccgggatgacacaattttagtgtctgtcgtggtctgtttcaggtctggtgcttgtttttgggttggacgtttgtccttattctgttcttttaatccgtataatttgtgtaatttgtggatatttttaaaagactggatcacggctcgcaggccgatatagcttggtggacttgtttcggggctggatgactgacccaaacggcgctatggcaagcgccactacagtaaaggcggtcgctgaaccgccttcagggcagattcttttgtcctttaccttatccggcaaaattaacttgacataatttattataggtgattatctggtgactgtcattaagtttcgttttgaaatctccaaggaggcagatatgaaacgatcgcttcttatattggcgctgcttgccgtcacggcatcagccttcgccgccggagtacttatcaacaagcaaaacactcagtttcagattgctcaattgcggagctgcagctatgaggtacaaatcaccaatcaggttgcggaagtgcaagtgaacgaaacatttgtcaacaacagcaacggttttttctatccccgctactattttcccatcccgcgaggtgccaacgcgacccgcttgagatggagctatggcgacacctggcaggaagcgtcaatttcgccggtgccgcaaaacccacagggcggcccgtcctcttttccggatgaatttgtcgtttatatccagttgatgccggtagttttcgacgtggcagactccatggccgtgaatgactccctgcggctggaactctcctacgtgatgatgttgagttacaatttcggcagcgtaaccctcaacctcaagaacgactacagcctgttgcagagcactcctctggatttgcagtccctggatgtgaacgtctcgtccgacaaagagattttggatttcaacatcctgaatgtgacagccagcacccaacacacggggcacagcgccagcgggcattactccctcgccaatcaacccgccaccgccaactatgcctgccagatactgctatcgaccgccgaactgggctcctggggcttgagcaccttactggatgatgtccccgatgagggggcgccggggtttttcgtcttcaatcttgaggaggaaagccttccggcggattccaccttattcctgcggctgaacatcgtgatcgacgtatccggctccatgacctttgaaaaccgcctggaaaacgccaagactgccgcgatctatgtgctaaaccatctggaacccgaggattacttcaatatcatcctctttgaccaccgggtgcgccctctctgggattatctccactccaatacagacagcaaccgccaagccgccatttcctatatccagaattatgagattaccggtttgaacggaaccaacctctccggtggtttgcaaaacgcgctgtatcagttttccccaactcccccgggagtcaaaaactgcgtgctgctcctttccgacggccaaccgactgtcgggatgacggatacctaccaaataatcaacaatgtcaaccaacacatcaccaactataacatcgatccccgcatcttctgctttggagtcggcaccgaggtcaactatcatctgttaagcgcgctgtctcaatacacagacggcgtcaccatctttctggaaagctccgagatcgtcaatacggtcagcagcttctacgacgtcatgcgcaatcctctgctcctcgatcccgaactggccatagtcccgcccgggatcacggaggtgtatccctatcccatcccctctatttatggaggaatccagtatcgcatcgtgggcaggtattcgactccgggaacctatcagatgacgatcagcggcgcgcaccagggcacccagttgaacctgaactattcccaaaaccttagcggagaggaagcccccggtttgagcttcattcccaagatctgggctgcttccaagatcgacaacctgatggtggagtattattcctatccgccaaattcggcggaagcgatcgccctgcgtgatcaaatcaccgagatcagccagagtttcggggtggtctgcatcttcaccagtttcacttccgatccgcccgacacggatttggatgacgtggtccaacccgtggtaaaaatgcccctgcgcctgctgcctaaccagcccaatcccttcaatccctccaccctgctctgttttgaagtgtccgccgaactggagggactggcagagatcagaatctataatctgaaaggacaactggtgaagatcctcacggtgaaggttgacggcaaaggcaggtacagcgtgccctggaacggcaccgatctgactgagcggtcggtttccagcggggtttatgtctatactgtcagatttggtaagtacctgctccacggcaagatgacacttgccaaatagagccggacagatgccctttgcggcgctatggcaagcgccggtactttgtaaaggcggtcaccacaccgccttttttctatccctttttaagctcttttacttcgtgttaattcgtgttaattcgtgtaattcgtggatgaacttccaaaagtccaaactgcttcagccccagtatttgcggtccaggctgcggtattggatggcttcggagatgtggtcggatttgatggcagccaggccttccaggtcggcgatggtgcgcgccaccttgaggatgcggtcgaagacccgggcggaatagcccagcttgtcgatcgcgtgctgcatctggctttggctgggggcatccagcgcgcagtgtttgcgcagcagtttggagttcatctgggcgttgcagaagatgccgtcgttggcatagcgttcccgctggatggcgcgggcgcggttgaccctggcgcggatgtcggcggagctgtcgccggaaggcaaagatgccagatcggaatagctgaccgagggcacttccacgtggatgtcgatgcggtccagcagcgggccggagatacggctgcggtagcgttggatggcgccgtattcgcaggtgcaggggtgattggggatgctggagccgaaatagccgcagggacaggggttcatggaggcgatgagcatgaattccgcgggaaaagtgaggctttgggaggcgcgggagatggtgacgatgccatcctccaggggctggcgcagcacttccagcacgccgcgcttgaactccggcagctcatccagaaacagcacgccgcgatgcgccagggatacctcgccgggacgggggaaggttccacccccaatcagggcgatatcgctgacggtgtgatgcgggctgcgataggggcgggtggtgaggatgccgttgtgaaaatctttggagtatcccgccacggagtggatcttggtggcctccagggcttcgtccagcgagagttccggcaggatggtgggcacacgccgggccagcatggtcttacccgagccgggagggcccagcatcagcaggttgtggcctccggcggcggagacctccagggcgcgcttgacctgaaactgccccttcacgtcgtgcatatccacgggaaaggcgttcagggaggaaaagatttgctcgcggtccacctcagcgggcggaatcttcgcctctccgcgcagaaagagggccgcctccttcagcgaagtgaccgggatcacctgtaatccttcaatgatggcggcttccccggcattttcccagggcaggacgagggtatcgatgccatcccgtttggcagccaaagcgatgggcaacactcccttgacggggcgcaggttgccatccagggaaagctcgccgatcagggcggtcttggtcaggtattccaccggaatttgcttgatggactgcagcacggcgatggcgatggggaggtcgagggcggcgctgtccttgcggatgtccgccggggcgaggttgatggtgaagcggcgcacggcggcgtcccagccggaattgcgcagggcggcaaagatgcggtcctggctttccttgaccgcgttggatgccatgcccacgatggtgaccgcgaaaacctgagaggtggcgtcacactccacagtgagctgttgcccgtctatccctatcgtggtgtaagtgcgcactattccaaccatttgaacctacccttgtttggtattttgctttgttttgcagtaaaatagcggtcggcgcaacggtcaagggaaaagtgcgggaggagggtgactatccgcaaattacgcgaatttacacgatttattagaagtggaaaggtggaatggtgaaaaggagcctcgaaataaacccgaccaggccatataccgtgtcatcacgggctcgacccgtgatccagtcttttagaaacaaccagccagcccggaaacaaacaaccccagactaaaagagtgtcatcacggccttcgagccgtaatccagccttttggaaacagcaaccaagccccaaaacaaacattccacctttcctccaattctacaattcttcccccatatatataggtgctacgacccctttatcccaggcaggaagcggattccagggcaagtatataaatatacttgcccgtaattgcgcggccatgtatcggcctcttccctttccctcgcgcgtgggtggccctcatttgcatcaccttctccggcgtgggtcttcctggggtcaaccttataatttataaggatgacctaaggatgattagctaacctgttgatggtgcaaatgatagcaacatgggaggcaaaaaagtatgtcttggagtttgcccgttggtttccgggctggctggttgtttccaaaagactggatcccgggtcaagcacggcttgcccgggatgacacagcatatagcttggtggacttgtttacaggctccatttcacctttccgcctttccacctttccacctcaaaaacacctttccacgtataataattcgtgtaattcgtggactattatctaacaggcttgctccagacgcgcccatcttcgcggatgatccagtccggggagccgcccttgccgggcagggtgaggtcaaatccggcggaggtggtttccaggcgtggtttaatctttttgccatcccgcagataggggcgcgcgcccagttgacgcagctttccagcccagcgtccgtgatccataaaatactgtttttggaggtaatagatctgcctcagatattccttttcccgctcggaggcaggcggcgaaaaacccgggtctgccgagccagcgggcgcggcggagaagaacagataaccccaacgctctggatagtgcatcgcgatcagtccctggggactccaaacccagttgcgttcaggcataccgggcactttggtgtagttgccgtccagccactcggtatcccattgcacgcgggagaagttgagccgccaataatcgccgtcccggggcgggcaggggacatgggcgaactcctcaaaggccggccagggcagaaacatctccacgctccagccgctgtcgaggtcactggggtcgttgagggtgccgtcgattccgatggcaaagcttgctcccttgatgtcccagccgttcagggggctgtgggcgtcgcggtaaggcttgaggatgaagaggtcccagagggtgcccagggcgttgatctccagttcgtaatactcgtgggtgtcgccgttggggtcgaggaagacctcaaaatcgttgtcgtaaaagatcaccgcgtcacgctcggtgaggcttgcccagatgtggggctcctccagccaggcgccgatgtagagtccggagtcatcccagagcatcttgacccaggtatccagatggggcgcgggtttgaggtcgccctcgatatccgtgaacagctccgtgggcagggcaagttgccagtcgatgtcgtccaggctgccatccaccttgatcccctccaccgttctgaacgcggtgtaggagcggggcgcgaactcgcccacgggcaggggaaaggtctcggcggcaaggacggaaaggatgaacaagagaacggggacaagcagttttttcatgggatagctccttatggttcgcggcaggctgccagcagctcacccgcggtggcgacggccagaccgacacaggtgtccgccgcgccgtaatagatgaagattctggtgccgggcaacactgtgccgtcggcttccacgctttcggggatggcggcggagggaaagaccacattgggcacttggccactcagttcccaggattcacgcggttccaggatgttttgcgcgctcctggcggttacgttccagggcaaagcgccatcgtgcagcgaaactcccgcctgatatatgtttacgctggcaaagtgggtcgcaacgccgtggtagatatgcagccagccctcggaggtcttgagcgggggcgggccggagccgagcagttcgtcccaatagtggggacggccgctgaagacgggggccacgggctcccaattaagcagatcgggcgaggcgtagcagacgatggtggctccggaacgcactccatccgcgcccagatggtcattgggacgggcaaagagcagatagctgccgccgatccgttccggaaagagtaccgcgttgcgcagatcggcatccgccagcaggcccagatagcgcaggctgcggcagtcttccgtttcaaacagcgcggagcggcagcccggggcgcaatcgatggcgcaggtgacgtagatgcgcccctccaggcgggtgacgcgggggtcgtagaggtggtaaatctcatagggaaaagcttccagcccgctcaccgccacgggatggccgtccacgcggaaactgacgccatcggctgacgcggcgcggacaaacagggtgcgcctgccgcgatcctgcacgcgcagcagcagaacatactcgccgccaagcttgaccccaccggggttgaagacagagctgacatcgcgcggcaaggcatccgaaacggggatatccgcccgggtgaggacgggattgcgcggatggcggatcatagcaggcctttcagctctttgacgcgccgctcacactcggcgaaatccctggaagggaaatcgctccagaacagctccgccatgtaaagcatgcggtcatagaccatttccttcagacggcggctattggaaatccgctccgtccagaggttggcctgtccgcccaggaagagtccggggttggaaaacctgaacttgccgaggtccagcccatagacatcccggctggtggtgactccgaagcctccgggacagtccgggtcttcgttatagcgccagtcaaaatagaggcggttgttggggcagatgatgtagcggttgccgttgtcatgagccatgcgggcggcatccaccccgtctccgcgccagcacatggcgatcggccggctgtcgatcccgccgtccagaatctcatcccaaccgatcacatccctgccgcgtttttgcacatgagccaccagttgatgcaaaaaccaggcctggaggtgttcttcatcccgcaaaccctcttcccggatgcgccgctggcaacggggacactctttccagcgctccttgggcacctcgtcgccgcccagatggatgtattggccgggaaagagttccaacacctcatccagcaattctttaaggaaatctatcacgctgtccttaccggcacagaggatgtccgtggaaatgccccagacgttgagcggctcaaagtcccgggggaagcaggccagatccggataggcggccaggatcgccatggcgtggccagggatgtcgatctccggcaccacctcgatgccgcggtcccgcgcgtaatccagcaccaggcgcacctgcttccgggtgtggaaaccgccgtaaaggctgccatcctcctctttgcgccaggcgcccacttcatgcaggcggggaaagcgtttgctctccagccgccagccctggtcatcgcttaggtgccagtggaatttgttcagtttgacggccgccatccagtccagatagcggcggataaccttgtagtcaaaatagtggcggctggtgtccagatgcagtccccgccaaggaaaggagggccagtcctccaggtcgaagcagggcagtcgcgcggagtcctttttgccttggcgggcagccttgtgccaaatctgccgcaggctctgcacgccatgcaccaggccgggatagtcctgcgcgctgatcaccgtgtcctggggacggctgactatcttgtaatggccgcctggacgctttcccttgcgcggggtaagttccaggcttacgctatgcggggtttcgctgtctggatccatgaagtccaacagcttgctgtaactgctttccaggctggaggagagatatctgtgggcactggcggagagttggggacagcacatgataataagcaacgggctggggcggaacgtctctttgagggggtgatactttttgggcttaggaagcatgtttcctaccttatcaacagggttttgatctggaagggcttgaaactgaggcggatgccccgctcgccgatgggctgcgcttcgctgtccggcaaggggttttccaacaggtcgcagacgctccagagatggtggccgggtgccaaaagagtagcctcggcggaacctcccgcgcattcatggaggcggacaacgatgccgccgccggattcagcgggcttgatccaatcgagggtcacggcatcggaaagcaggcgcacgggcggacggggcagagggttggcgagggcgggcacttccaggcagagcagatcggcgcaaaaggcctcggcagcctgccagagccgggagccggcatagtctccgctgtggaagtaaaccgcgtaggaatattcgtgttcgcccaaatctgccagggggtcaacgtcggcgggagagcgcaagaggttgaggtccatctcgttgtcgcgcacatggcagccgaacttgtcggcgcagaaaatggcgcagccgcggtctgcctgggaaagatcgaagaacttttggaaaggcacctcgaagcgcgcttcctcccaggcgttgcggggcttggcgctgcgtcccagcatgccggactggatgccaaagctggcctgggcagagcggacatcgggataccagtgcgcgcgcagcatcttgtgcttttcctgccagtcgatgtggtgcgagacgcgcagcaggcgctctccccggcgcagttccagggtctgccgcaggatggaggagcccacgcgcaaggtctggacaaggcgggcaaattccccgggcagggtgtagctttgcgccgcttccaggctgatcccctccgcaggcgtgggtgtggttgtgcggtaaaagtggttgatatcccaggcgccccagttgttgggttcatcctcccagagcagcaggcggtttgattctgaggcgaggagcggcgttccggactccttgtcccacacctcgctcacgcttccctgctgggttacggtcacgcgcaggaactcattttccaggcaatagccagccagaggaataggcttgggaatctcccccactccggctttgaaggcgatggaacccactccccaggcgggcaggctggccttgaccagcgttccctccggggtggagcaacaggtgagggggacgccgtcttccccgcagggcagcaggccttcggcaggggcgggaagcggcagccattccgtcttggatgcgggagagggattgagcagcagccagccctggccgggtgaggcttccccggaggtggtaagtccggcggcgcgctggttcagaaagtcatccaggattttgcggttaccggcgcagatggcactggcatcgttataaaccatgccaatcgaggagccggggatgatgtcgtgaaactgcagcaacagggtatcctgccacacggggcgcagttcccggggatattcttccccgccggtgagcgcggccaggtattccgccgccctgagcagcctttccgcccgcgcattgtcccgcttcatccgcgcctgggtggtgtaggtgccgcggtgatactccagataaagctcgccgggcatcacaggcagcagggagcggtccagagccttcagatgctcgaaatagtctgacgacctggcaaagcggaacttcggcacgccctccagatcgcgctgccggaggccatactccagatgcgcctcggtgggcccgccgccaccgtctccgatgccatagaggttgaggaaggcgtcgcaaatctcgctctgggcgaagcgttcctccgtcttcaggaaggcggagggattgttggaaaagttatagtcgtgggtgggcagttgttgcgccagtacccggctgccgtcgatgccctcccacaggaagaggttgtgggggaaggcgttgctttcgttccaggatagcttttgggtcataaactgctccacgccgcagcccttcatcagttggggcaggttgccgctgaagccgaagcagtcgggcagccagaggacatccggctccacgccgaactcctcacggaaaaagcgtttgccgtacatgaactggcggatgacggactccgcggagatgagattggtgtcgaactccacccaggacgccccctgcacctcccatctgccttccgccacgcgctttttaacctgcgcgtagagagcgggatacagctccttcatccagagatagagctgggcttgggaggcaccgaagatatagtccggataccgttccagcaggcgcagcgcgttggcgaaggttctgccgcccttgcgtttggtctcgcgcaggggccagagccaggccagatccagatgggcgtgaccgacgctgaacgcggtgagcgcgctggcattagctggcttggcgagcaggtcgtccagaatgcccgcgcaggcttccctgtcgctgttccagaggtcgcaaacccggttcagtccatacaggatacgctgcctgcgggtctgcccattgggcaggctgagcgcaagatccagcagcagttcgatgtcccagagtagcttgcgcagccggggatcacgccagaccaggctgcactcccgcagcctatactcgctttttcccccgccgaaaaggtcgttggcggcggcctcgatcagcagttccacccgttccccctcttgcaggggcaggggaacgaaatacttggcggcgttgtgataccagtccaccttgggggtcaggccctgccagggagtttggcctttgaacacgcaggcttcgccgtcacaatcgaaccacagccccagttccatgcccgcgggggttgccggagcctgcccgctgatcctgaaccaggcgctctgccagggtttgccccacagtccgccgacttcgatctccttgaaactcatcgaggcgagcttttcctgcgggatgggatccgccgcgctcacatactccgcccgcacgggcagcttttccccgtgcagcagcgctttctgcctctccacgaggcgtttgatccgctcgatatggatcttttcgttgtgcattatttctccatggggaccacgatgccccggataatgatattctggaagaagacaaacaccgccagcgtggggatggcggcaatcaccagggcggcgtagcccactccgctgctggcgcgctgcaacagctcgtagatgtgcaccatcagggtccacatcgactgatcctggcagaccaggaaggcaaaaaggaagttgcggtaagccgcgttgaaggcccccagagcgatcaccgccaggatcggcttgctgagatacatggtgaactgccagaacatgcgcagctcccccgctccgtccaggctggcgctttcatagatttcccgcggcaggctgtcgaaaaagcccttgagcaggaagatgaagtagccatccgccgcagcgggcaagaccagcgcccaaaaggtgttgagcaggttcaaacgcttcaacatcagaaagttcggaatgcccatcaccatcgccgggaaagccatggtgagcatgaagatcatgatgacctgatgcgtgaagcgcggtttgaagcggctgagcgcgtaggcggccagcgggttgaccgtgaccgcgaagaagatggagagcagcacgtagatcagggtgttgcgcagcgagcgggagtccgccagcatctgatccagcgccatggcatagttgcgggtgagaaactcccgccggatggcgcccttgcgctccataaaagttcccctgtcgatctccgcgctggggaaagggactttcccaaagcccgtgtagctgcttccccaagcccggttgagcgcgccgagggagccatacttatcctccaaccagtccctgaaccccgccgccacgctcactatcctgagttcttcgggagccgccctggtctggatatacagggccagatcgtccacttgcgcgccgctgtccggcaggcggacgggaatgccgacctgctcgaaactgctgtaagatgcctgccaggcggtgttcagcaggctgatcccgccgtacttttccttcagcgcccggcggaatcccggctccgcctccggggaaagccgcagatgctggatggaaagcacttcccgcgcatattccagccagtaggggcgcagcgggtcgccccgggcgggacagctttcgggcaggatcacctggctccaggtggcgtagctggtgcccaactccttgttcagcttgtccagattgccctggaaggtggggatcaggatggcgttgacaaaggttccgtccgggttcatatagagccgctgccagtccgggcgttgctccttgaagcggcggaacctgcccagatagccttgggtggagctggcaaagttgcgcaaaaagatgtccttttcctccaccgaaatctcttcccagcttttagccccggtgttgtagcggcggttgaaggccgccaggtcgccgccgttttcctcgcgcagcagcttgcggtattgcctctgcgccagcggatagacccctcttccgtaatgctccgacacatagtagaaattgactccgtagtccccgccgcgttcccgcagaaagcgcttccaatcctcatacacagcgggattgccgtccgagggcagttccacctcggaaaaggcgatccagccgcccggatagttgtccatcagacggctgctttcctcgttgtagcggctttccagatacttttggtagagcgcgccgtcccggtaaacatagcgcggcaccagatccagctgggtgctgtccaccgcgctcttgaacgatgaggaaatcatcagcaaaaagggatagaccatcgtgaccgagccaagcagcaggatgacgtgcaaagacgcgttcagcgcccgcaccttgaagctttgcctgcccaccctgccgatgatactcattcgtcctcccggcccgccacgcggaactccatgcgtgagatatagcgtatctgcagcaccgtaaagcccatcagcagcaggctcagcatccaggccatggttgtcgccagcccgaaacgcagatagaggtaagccttttcaaagatcatcaggtctgccacccgggtcgcctcgttgggcccgccgaaggtcatcaccaggataaagtcgctggattgcgccgccacgatgaaggccgcgatcagttgcacgatgatcagcatcttgaggctgggaaacacgatatggcgaatcttggcaaaaaagcccgccccatcgatgtccgccgcctcgtaaagctcattggggatgttcttgagcgccgccaggtagatcagcgaacccggccccactcccgcccaaaccacaggcagcaccacgcaaagcatggccagtttttcatcctgcagccagcggctcttgggcaggccgaaagcggtcagcaactggttcagggcgcccacgtcacccggggcgtagagcagtttccagaggtagatcacgatcaccccgctgatcaccgccggcaggtagtagatcacccggtagatcatcttgccgcgcgaaacctcctgcagcatgatggccaggatgatgggcgggaaaaagcccagtcccaggttcaacgccatgtaatacaaggtcttgcccacagaggaccaccaactgggatcgaaaagcatctccgcgaaattggccatgcccacaaaggggctggggcccaccaggctgtaatcctgcaccgccataacgctgccgctgatcaggggcacatatttccacacaaagatgctgatcgccgcgggagcaagcagcagcaggacgcagcccaggcgtttggtctgctgtcccccgccactcccccgctccggggccagaatcttccacacccgccacagcgtgaagcaaaaggcaaaaaggatcagcgccgccaccacggccgcccatcgttcccgggctttgcgcgtggcttccggcagatggccgatcatctccgcgttggtgcgctgttccgcccgcgccagcacccgcttgatggcggctttgcgtgccgcggggtcggtgccgagcttgcccgccagttgcaggccgatcagctcgtccagcggataggtcatgtattcataaactttctggcagttgtcgccaaagggttcgggacgcccctcgcgcatcgcggtctcaaaggtctgcagccagcccgccggggcgtagcgcaggtattcgccatagccatagcgctgcaggaacaccggattctgcatcttgccatagccgttttccaccatcaccttcatgcggatgcgccgcgcttcctcgctgtcgtagaagcggatgaaagtccaggccgcctcgcgcacctcccgcgggtcacgctgtcccagtccggcgttgttgctgattccggcaccggaaaagattcccatcatgcggcagttgatttcgctgcctccgcgtccggaaggcccggcggggcttggcgcgaagccgtagaggttgggatcgtatttgccgcccatgttctgctgcgaaaggtagtccatgcgcatcccgatcttgccttcctgccagaggttgccccagttgccgtcgcggatggcgaagccctgctggagcttgcccgcggagtccgtccaggggcgcgtggcgaggtcgaggtaatactccatcgcctccacacccgcctgcgaggcgaaggaagcccgccactgcctgttgtccgcgtcgtaggtcaccgcgtcgccgcctgccgcccacaaaaagggcagccagtcataagctgcctgcgggccgatggcgaggatggtaccataggttccggccttgggatcggttagctgtttcgcataggcggcaaactccgcccagtcgcgcgggggacggttgggatcgaggccgcatttcttgaacaggtctttgcggtacatcaggacgcgcaccagggtctcatagggcaaggcccagacccgctctttgctatccccctttttctgcctgcgaatcacctgccagacgggtttttccacccgcagggcgaggtcttccgcgctttcccgcgccataaactcatccagcggatagagcaggttgttttggatataggtgtcgctctggcggaaatttacatacatcacgtccggcgccactccccccgcgatcgccagcagcgggccggaatccaggtccatgccctcgatcttgatgccggaaaaggagcgcagttcgatccacggatatttctggcggaaggcgttgatcaccgccaggtcggctttgctgttggcatcggtatgccgggcgtcgggcagttcaaaaaccttgagcaccaccgttttgcgctgcgccgccgcggaaaagggcagcaggcaaagcagcagaaccagcggcgccagacggcggataccagcggaacgccgtccccgcgtggctttggaacactgtaaagctctgaacaaactcatcattttaccaaccgctgcatcctgcccgggcccgctctacaggtcaagtaaaatcaggttcgtgctttttcagcccctctttctccagaacaggcagcagggcgcggcggtattgctccagccggaaggcggccaggctgggccaggcgccgggattggccctgtaaaaggggtcacgtgccaggctcttgaaggcttggggcgaaggcgagatcagcgtgtatatatccagcttcatgtcctccaactccacgtgcagcaagcgtctgtcaccaggatcaaccgcgagggcgcgaataccgggatcgacacgcatttgtttgattaacaaatttttaacatagaaactggtgcagaatatccgttcaatctggggcatgaggggcagtatacccccgacgggcacggtggaaaacacctgcagggaactgtcggaggcagagtttccccgcctggcagcggtttgaatgatgtcaacgacaccgacgttatgtttgcgcaaccaactcttgatcaagtgcttgggtatttccttgtccgctgcgtccaaagaccagttttgcccaaaaacctcctgcagcaggctccagaacgcgttgttccggctgccgtaatagtagtccatgtctccttccggcaatctttgcctcagttgcccgccaaagctgacgtaccgcgggggcggaaaactgcccacgatcagccagcgcgttgtgtccgacaggagcgtctcatcatgcaacccgttgtagggatgcttccccaccgggaaaatcaatatgtttctgtcttctgtttcatgctgttgcttcatgctgttctccatatcatgctgcagggcagcttatctgatccgccccgcaagtcaagtgaaatattccgcttttgcttcacgccgccatcccttgctgctcctcgctttggtgccatcatccttaggtcatcccttaccgggttaaggatgactgccggaagacccccggcaagccagatattgcaaacgcggcccccatatatataggtgctacgattaggccgtggaaaggaaatagaacgcggataatataagggctgccgaaatccgttcggcagaggcaacggagttgcctgtttggaagagacctccggtctctgtggcaaacggactttgccaccccaaggcactggctcccgggtcagtcagctcattcatctcccgcaaaaaggttgtccgccacatcggaaggcaggatggagggggttccgcggcgggcggcaagtttttccgcggtgcgtcccagcaggaaggaagcgttgatggcggcttcggggatgctcaaaccctgcgccaaaaaggagatgatgatgccagccaggacgtcgccgctgccgccggtggcgagcccatcgttgccgctgctgtttaccatagtggtttcgccgtcgtgatagacgctgaagtgggatttgagcagcacgcgggccttatggcgggcgacaaattcacgcagaggggagaggcagtccgtttccaggctgtccttggggatgcccgccaggcgggagaactcgccccagtggggtgtgatgaggatgtttggcttggccagatacttgagcagggcggggttgtcggcaatcaggttgaggccgtcggcgtccaccaccagaggccgcttcacctggcgcagggcgatctccagcagacgcagggaatagctgtcgcgtcccaggccgggcccgatcagcaccgcggaggcttcgcccagcaagctggcaagctgtttggtatccggcatggcgcgggagttttccgggatggagaggctgagggcttcggtgagcctgatgaagtagaagggctgcagttcaggccgatggagcatccagacatatccggcaccggcgcgcagagcgccccgggcggccagcagcgaggctccggtgtagcccggcactccgccgaagatatagaccttgccgaagtcgttcttgtggctgtccggatggcgcatgggagggcagaaatcctcttcggtgaacagtttgacgctgggcaggccgtggtagtaaaagtggggaatgccgatggggacaaagctggtggggccgcagacttccctgccgtagcccaggacgtgtcccagcttgagagaggcgatgcagagggtggcatcggcgtaaatcgcgtcgccgcaccagccggtgtcggcatccagccccgaggggatgtccaccgcgatcacgcgggaggcactggtattgaccacttcaaaaagggtgaggtgaaagtcgtccggctcgccgtggaagcctgtgccaaagatggcgtcgatcaccaggtcgctgtcggagacgatctcttccagcaggggcaggtcttccaggctggaaatctgcagaatggggatgccgagggcgcggcaaagctccagattggccttggcgtccggggagaggtcgcctttcccgatccgcaggatcaccacttcgcagccgccctggtcgagccagcgggcaatcacggcgccgtcgccgccattgtttccgcggccgcagaggatgcaggtgcggccttgcagccattggggatattgcgcggcgatcaggtcggcgcagcctttgccggcggtttccatcaggacccgggaaggcaggccgaattcctcggcgcagcggcggtccaggtctttcatttgggaggaactgagcaggtattgcatggtttacctcttgctattggattggatgggcagtttgatctcaaaggtggtgccttcgttgggaacgctttggacaacgcgaatctgcccctggtgatattcttcgatgatgcgtttggccaggctgagtcccagtccccagccgcgggtcttggtggtgacgccgggatcgaagattcgtttccattgtgagcgggggatgcccttgccttcgtcgcgaatctgcaaatagatgtgccgttcggatttggaggcggtgatgatgatgctgccgccctttttggccatggcgtccacgcagtttttgatcaggttttccagagtccatttgaagagctcgggatccagcatgacctggacgccctcgatcttggaaatgaggtggatgtcgatgcgggagcccagatggggcatgcgcttttggaagtaatccaccgtctccaacaggatggtgttcaattcggtggggacgagattggtctgggagcccacctttccgaagcgggaggcgatcatgcgcaggtggttgaggtcggcggtcatgtattcgatgatgcggtccattccgggacggtcctgccctgtcccgggggattccttcaggtagtcgatccagcccatcagggaggtgatcggggtaccgaactggtgcgcggtttccttggccagcccgatccagagggtgtccttttcggtgcggtgcagcaggaagagcccgtaggaactgaaggcaatgagcagcaaagccaggatgatttccagcacgaagacgctttggatgcgggaaagcgacttgggcgcggtgaagtagatgtagccgaggctgtccgccgcgttgctgaggggaatctcggtcatcctggccagcctttcggacacctgctcacgctggaaaggcgggatatcataccaggggagggattcggggatgtccatgttgcgccagtagagggggtttttatcgcggtcggtgaccaccacggaatagtcgatattgctgaggaactggcgcagggaaatgggcttggcgaagaagacatagttggtgagcacgccgttggcgtcgatgggtatcctttccatgctgcccagatgcgaaaagaggatttggcgggaagccgggctgagttcggcaaagctgctgtctgaagaaactcccaccctgctccagtagaggggttccagatcggtatcggtgatgatgatgggcagtgggttgtggggcatgaattcgtaaaggatgtagtctccgatggaggagttgaagttaggcttctgcaccgatttgaagtagtttgagctcagctcgccgatcatcagggaatactgttccgcctggcgcaggtagtcatctgaataggcgatgtactgagcgaagatgcggggaatatattcctgctcctgcttggcgttgtggatcagaatctgggtgtaaaccgcaaagatgccaaagactacaatgctgccaaagatcaggtagaggcgcagggtgcggaagctattggtcttctttcgcgaggttccagagggcgtcgagttcttcgaggcttgcttcatggatattggctccgttttggcgatagtgctcctcgatatagcggaagcgttggtggaatttccgggtggtttccttgagcgcgccttcggcgtcgatattcagcttgcgggcaaggtttaccagggtgaaaagcatatcgcccagttcttccttgatcgcggcggcatcctgggaagccagggcggattgaaactcgtcctcttcctcccgcagcttgtccaacacgggtttgacggaggcccaatcgaagcccaccgcggcggccttttcctggctgcgttgagcttgaatgagggcgggcagggcgcgggggatgccttccaaaacgctgatcctgtcggtcttttccgctttcttgatgcgctcccagttgagtttgacggcttcggaatcacgcacatcggcgtcgccaaacacatggggatggcggcggatcagcttggcggaaatcctggtgaggacttcgcggatgtcgaactgatcttcttcctcggcgatccgggcttgaaacacgatgtgcagcagcaggtcgcccagttcttcgcagagcgcctggggatcgttgtcctcaatggcttccaccgcctcatacaattcttcgataaagtttggcaccaggctctgggaggtctgtttagcgtcccaggggcagccggaaacgggatccctcagtcgggagaggatatccaccagttcttgaaattctttcataagcctacctccggggcgagttgccgcgggcgggtgtgcagggtggcgttcaccgcggcgaaatagatccagacaaagtagttccagcggatggaactgggcacggaagacgccggcacaaagagcagcagggagatgaaatacatcagatagcgctgcctgtccgcgccggtggaattgcggtagagcttccacagccccaccagccagtaaaaatagaggtagaggaaggcgagcagtccgggcaggccccagttgcccagcagttccatgaaatagttgtgggcgttggtgatgccggccgtgcgatatatggcgtcgccctgcatgtgaggctcatagtttccgcctcccacgccgatgaaacctgaacgggctgccatgtcgagggtttccttgatgagctgcacgcgaatctgtatggagctcatgtagatggacttgcgttcgctgcccagggtgccggtctgataattgaaaaccttatacaacagatgcgcttcccggttatagcgcgcgtagatgcctcccgccagcagggccagagccagcagcaggataagcttggtcttccaattggtctgaacgataaagtaataagccagaaacgcccccaaggccagcatcgcgatccgggcgccctgaatcaccatgatcaccaagatgaccagcatcagaggcgcgctcagcgccgccagccagcgtttcccggaaatccggggcacaaaaagcaaaaaggggctgaaaagcagcaaatagctggccaggttgttttcattgtaaaaggggccggaaggaatgaagaagcctttgccgtagtggcgcgaaacaggcaggtgcttaaaggtgaacatctccaccagcgccgtccccacatagagcagcatcagcagaaagagaaaccagtggaatttgcgcaacagcccggggtcttcaaacagcttgtcgtagatgaggaaaagagccatatagcgcagtatccacgccgtgtagctgagcaccagg

SEQ ID NO: 11 - DNA, 12 bp, synthetic construct
  source 1..12: mol_type = other DNA
acttgtttaagt

SEQ ID NO: 12 - DNA, 16 bp, synthetic construct
  source 1..16: mol_type = other DNA
ggcaccgagtcggtgc

SEQ ID NO: 13 - RNA, 30 nt, synthetic construct
  source 1..30: mol_type = other RNA
  variation 8: note = n can be a or g or c or t/u, unknown, or other and represents part of the bulge in the DR:tracrRNA duplex
  variation 9..12: note = n represents any complementary set of nucleotides that together will base pair to n in positions 17-20
  variation 13..16: note = n can be a or g or c or t/u, unknown, or other, and represents a linker sequence
  variation 17..20: note = n represents any complementary set of nucleotides that together will base pair to n in positions 9-12
  variation 21..23: note = n can be a or g or c or t/u, unknown, or other and represents part of the bulge in the DR:tracrRNA duplex
gyyyyagnnnnnnnnnnnnnaanttrrrrt

SEQ ID NO: 14 - protein, 7 aa, Simian virus 40
PKKKRKV

SEQ ID NO: 15 - protein, 16 aa, synthetic construct
KRPAATKKAGQAKKKK

SEQ ID NO: 16 - protein, 9 aa, Homo sapiens
PAAKRVKLD

SEQ ID NO: 17 - protein, 11 aa, Homo sapiens
RQRRNELKRSP

SEQ ID NO: 18 - protein, 38 aa, Homo sapiens
NQSSNFGPMKGGNFGGRSSGPYGGGGQYFAKPRNQGGY

SEQ ID NO: 19 - protein, 42 aa, Homo sapiens
RMRIZFKNKGKDTAELRRRRVEVSVELRKAKKDEQILKRRNV

SEQ ID NO: 20 - protein, 8 aa, Homo sapiens
VSRKRPRP

SEQ ID NO: 21 - protein, 8 aa, Homo sapiens
PPKKARED

SEQ ID NO: 22 - protein, 8 aa, Homo sapiens
PQPKKKPL

SEQ ID NO: 23 - protein, 12 aa, Mus sp.
SALIKKKKKMAP

SEQ ID NO: 24 - protein, 5 aa, Influenza virus
DRLRR

SEQ ID NO: 25 - protein, 7 aa, Influenza virus
PKQKKRK

SEQ ID NO: 26 - protein, 10 aa, Hepatitis D virus
RKLKKKIKKL

SEQ ID NO: 27 - protein, 10 aa, Mus sp.
REKKKFLKRR

SEQ ID NO: 28 - protein, 20 aa, Homo sapiens
KRKGDEVDGVDEVAKKKSKK

SEQ ID NO: 29 - protein, 17 aa, Homo sapiens
RKCLQAGMNLEARKTKK

SEQ ID NO: 30 - protein, 4 aa, synthetic construct
GGGS

SEQ ID NO: 31 - protein, 5 aa, synthetic construct
GGGGS

SEQ ID NO: 32 - protein, 9 aa, synthetic construct
GGSGGSGGS

SEQ ID NO: 33 - protein, 15 aa, synthetic construct
GGGGSGGGGSGGGGS

SEQ ID NO: 34 - protein, 10 aa, synthetic construct
GGGGSGGGGS

SEQ ID NO: 35 - protein, 20 aa, synthetic construct
GGGGSGGGGSGGGGSGGGGS

SEQ ID NO: 36 - protein, 25 aa, synthetic construct
GGGGSGGGGSGGGGSGGGGSGGGGS

SEQ ID NO: 37 - protein, 30 aa, synthetic construct
GGGGSGGGGSGGGGSGGGGSGGGGSGGGGS

SEQ ID NO: 38 - protein, 35 aa, synthetic construct
GGGGSGGGGSGGGGSGGGGSGGGGSGGGGSGGGGS

SEQ ID NO: 39 - protein, 40 aa, synthetic construct
GGGGSGGGGSGGGGSGGGGSGGGGSGGGGSGGGGSGGGGS

SEQ ID NO: 40 - protein, 45 aa, synthetic construct
GGGGSGGGGSGGGGSGGGGSGGGGSGGGGSGGGGSGGGGSGGGGS

SEQ ID NO: 41 - protein, 50 aa, synthetic construct
GGGGSGGGGSGGGGSGGGGSGGGGSGGGGSGGGGSGGGGSGGGGSGGGGS

SEQ ID NO: 42 - protein, 55 aa, synthetic construct
GGGGSGGGGSGGGGSGGGGSGGGGSGGGGSGGGGSGGGGSGGGGSGGGGSGGGGS

SEQ ID NO: 43 - protein, 60 aa, synthetic construct
GGGGSGGGGSGGGGSGGGGSGGGGSGGGGSGGGGSGGGGSGGGGSGGGGSGGGGSGGGGS

SEQ ID NO: 44 - protein, 32 aa, synthetic construct
LEPGEKPYKCPECGKSFSQSGALTRHQRTHTR

SEQ ID NO: 45 - protein, 20 aa, synthetic construct
PKKKRKVEASSPKKRKVEAS

SEQ ID NO: 46 - protein, 21 aa, synthetic construct
GGSGGSGGSGGSGGSGGSGGS

SEQ ID NO: 47 - DNA, 27 bp, synthetic construct
  source 1..27: mol_type = other DNA
ggtggtagtggagggagcggcggttca

SEQ ID NO: 48 - DNA, 72 bp, synthetic construct
  source 1..72: mol_type = other DNA
ggtggaggaggctctggtggaggcggtagcggaggcggagggtcgggtggtagtggagggagcggcggttca

SEQ ID NO: 49 - DNA, 48 bp, synthetic construct
  source 1..48: mol_type = other DNA
tcgggatctgagacgcctgggacctcggaatcggctacgcccgaaagt

SEQ ID NO: 50 - DNA, 192 bp, synthetic construct
  source 1..192: mol_type = other DNA
gtggataacaaatttaacaaagaaatgtgggcggcgtgggaagaaattcgtaacctgccgaacctgaacggctggcagatgaccgcgtttattgcgagcctggtggatgatccgagccagagcgcgaacctgctggcggaagcgaaaaaactgaacgatgcgcaggcgccgaaaaccggcggtggttctggt

SEQ ID NO: 51 - DNA, 108 bp, synthetic construct
  source 1..108: mol_type = other DNA
ggtggttctgccggtggctccggttctggctccagcggtggcagctctggtgcgtccggcacgggtactgcgggtggcactggcagcggttccggtactggctctggc

SEQ ID NO: 52 - protein, 9 aa, synthetic construct
IEVMYPPPY

SEQ ID NO: 53 - DNA, 10 bp, synthetic construct
  source 1..10: mol_type = other DNA
ccccctcccc

SEQ ID NO: 54 - DNA, 30 bp, synthetic construct
  source 1..30: mol_type = other DNA
agtcacgacgttgtaaaacgacggccagtg

SEQ ID NO: 55 - DNA, 23 bp, synthetic construct
  source 1..23: mol_type = other DNA
tcattaggcaccccaggctttac

SEQ ID NO: 56 - DNA, 51 bp, synthetic construct
  source 1..51: mol_type = other DNA
  misc_feature 2: note = n is phosphorothioate modification
  misc_feature 4: note = n is phosphorothioate modification
  misc_feature 48: note = n is phosphorothioate modification
  misc_feature 50: note = n is phosphorothioate modification
gntntgtgagcaagggcgaggaggataacgcctctctcccagcgactnant

SEQ ID NO: 57 - DNA, 51 bp, synthetic construct
  source 1..51: mol_type = other DNA
  misc_feature 2: note = n is phosphorothioate modification
  misc_feature 4: note = n is phosphorothioate modification
  misc_feature 48: note = n is phosphorothioate modification
  misc_feature 50: note = n is phosphorothioate modification
antnagtcgctgggagagaggcgttatcctcctcgcccttgctcacananc

SEQ ID NO: 58 - DNA, 32 bp, synthetic construct
  source 1..32: mol_type = other DNA
ccaggccctcgtcattgcagcagcccccgcat

SEQ ID NO: 59 - DNA, 32 bp, synthetic construct
  source 1..32: mol_type = other DNA
ttctctacccacgaagggttacggcaaagcat

SEQ ID NO: 60 - DNA, 32 bp, synthetic construct
  source 1..32: mol_type = other DNA
agaccaattgtcatgcttgacacaactgctgt

SEQ ID NO: 61 - DNA, 32 bp, synthetic construct
  source 1..32: mol_type = other DNA
aattgtcatgcttgacacaactgctgtggctg

SEQ ID NO: 62 - DNA, 32 bp, synthetic construct
  source 1..32: mol_type = other DNA
ccatcctgtgtgcccctgatgcgatgcggggg

SEQ ID NO: 63 - DNA, 32 bp, synthetic construct
  source 1..32: mol_type = other DNA
atgcccacctgcatggtgatgttggactcctc

SEQ ID NO: 64 - DNA, 32 bp, synthetic construct
  source 1..32: mol_type = other DNA
tcctacattagagagtctggcaaggcagacag

SEQ ID NO: 65 - DNA, 32 bp, synthetic construct
  source 1..32: mol_type = other DNA
gagccactgcattgctaatgaaagaatgcaaa

SEQ ID NO: 66 - protein, 26 aa, synthetic construct
HHVTYERAGNESLDDLRSLCKICHDA

SEQ ID NO: 67 - protein, 27 aa, synthetic construct
QHVNYRRTGGREEPEDLRALCRLCHDA

SEQ ID NO: 68 - protein, 27 aa, synthetic construct
QHVSYRRAGGAETQDDLRALCRLCHDA

SEQ ID NO: 69 - protein, 27 aa, synthetic construct
QHITYRRAGGNETTEDLRSLCRLCHDA

SEQ ID NO: 70 - protein, 26 aa, synthetic construct
HHVSYENAGHETDSDLRSLCKTCHDA

SEQ ID NO: 71 - protein, 27 aa, synthetic construct
QHITYRRAGGNETVDDLRSLCRLCHDA

SEQ ID NO: 72 - RNA, 60 nt, synthetic construct
  source 1..60: mol_type = other RNA
tttagaagnnnnnnnnnnnnnnnnnnnnnnnnnnnnnnnngtgtaccgccggataggcgg

SEQ ID NO: 73 - RNA, 61 nt, synthetic construct
  source 1..61: mol_type = other RNA
gtgaaccgnnnnnnnnnnnnnnnnnnnnnnnnnnnnnnnngtattccccacgcatgtgggg

SEQ ID NO: 74 - DNA, 53 bp, synthetic construct
  source 1..53: mol_type = other DNA
gcggagaagtcatttaataaggccactgttaaaaagcttggcgtaatcatggt

SEQ ID NO: 75 - DNA, 53 bp, synthetic construct
  source 1..53: mol_type = other DNA
accatgattacgccaagctttttaacagtggccttattaaatgacttctccgc

SEQ ID NO: 76 - DNA, 53 bp, synthetic construct
  source 1..53: mol_type = other DNA
aaggagaagtcatttaataaggccactgttaaaaagcttggcgtaatcatggt

SEQ ID NO: 77 - DNA, 53 bp, synthetic construct
  source 1..53: mol_type = other DNA
accatgattacgccaagctttttaacagtggccttattaaatgacttctcctt

SEQ ID NO: 78 - DNA, 7287 bp, synthetic construct
  source 1..7287: mol_type = other DNA
ggatctcgacggagagcgttcaccgacaaacaacagataaaacgaaaggcccagtctttcgactgagcctttcgttttatttgggcgcgccaaaaaaatcgacctgcaggcgcgccgagctcgaattcggatccttctaaaccgcctatccggcggtacactttttaacagtggccttattaaatgacttctccttctaaaccgcctatccggcggtacacgctagtattatacctaggactgagctagctgtcaacattaattcctaatttttctctcccttatgcgactcctgcattaggaaattttacactttatgcttccggctcgtatgttgggggaattgtgagcggataacaattcccctgtagaaataattttgtttaactttaataaggagatataccatggcagctaataaaaaggcaaccaatgtcacgctaaaatcccgccccgaaaatctatcatttgctaggtgtttaaacacgactgaggcgaaattctggcagaccgattttctgaaacgccatacgttcaaattaccgctgctgattaccgataaggcggtcctcgcgagcaaaggccacgaaatgccgcctgataagctggaaaaagagatcatggacccgaatccgcaaaagtcccagtcttgtaccctgtcgaccgagtgcgatacgctgcgtattgactttggtattaaagtgctgccggttaaagaaagcatgtacagctgcagcgattacaactatcgtaccgcaatttatcagaaaatcgacgaatacatcgcggaggacggcttcttgaccttggcgaagcgctatgtgaataacatcgcgaatgcccgttttttgtggcgtaatcgtaaaggtgctgagatcatcgaaaccattgttaccattgaggacaaggagtacccgagctttaatagcaagtccttcaacctggatactttcgtggaggacaacgccaccatcaacgaaatcgctcagcagattgccgatacgtttgctggcaagcgcgaatacttgaatatttacgtcacctgctttgttaagatcggttgtgcaatggaagtgtatccatcgcaagaaatgacctttgatgacgatgacaagggtaagaaactgttcaagttcgagggtagcgcaggtatgcatagccaaaagatcaacaacgcgctgcgtaccattgacacatggtatccggactacaccacctatgaatttccgattccggtagagaactacggcgcggcgcgttcaatcggcatcccgttcagaccagacaccaaatctttctataaacttatcgaccgcatgattctgaaaaacgaggatctgccgatcgaggacaagcactacgttatggcaattttgattcgaggcggcatgttcagcaagaaacaagaaaaataagcattcaggaggaaacgatatgtttagtcaaattcttatcataaaaccaggcacaggcatatcccccaatattataatctcagaagatatatttccagttctgcatagcttgttcgtggagcacgataaaaagtttggtatcacgttcccggcatactcctttgataaaaagggccatctgggtaatatcattgaagttctgagcgaagataaagaggcgctggcgagcctgtgtctggaggagcacttggcggaagtcaccgactatgtgaaagttaagaaggagatcacgttcaccgacgactacgtgttgtttaaacgcattagagaagagaaccaatatgaaaccactgcccgtcgtatgcgtaaacgcggtcacaccgaactcggccgtccgctggagatgcatattaagaagaagaaccagcaaattttctgccacgcgtacatcaaggtgaaaagcgcatccaccggtcagagctataacatctttcttgctccgaccgacatcaaacacggctcgttttccgcttatggacttcttcgaggtgatactcatgcttagaaataaaatattggctgccatatcgcaaaaaatccctgaggaacaaaaaataaataagtatattgaagggctgtttcaaagcattgataagaaccaccttgctacgcatgtggccaaattcaccgaaaccaacagcccgggtaatataggtgcgtatgatatcttgtccagcgacatgaactgcggttacctggacaccgcaaatgccggttggaaagaaccggatatcgttaccaacgatgcgaaatacaaacgtcctcagggctttgttgctatggaaatgtccgacggccgtaccgtgatggagcacctacaagaagattctgcagaattaagacacgagatggaagagttgaccgataagtacgacgagatccgtgacggtattttgaacatgccgagcatgcagccgtatcgcacgaaccagtttatcaagcaagttttcttcccggtcggtggtagctatcatctcctcagcattctcccaagcaccgtactgaattacgaggtgagcgaccgcctgtatcgtagcaagatcccgaagattcgcctgcgcttgctgtcgagcaacgcggcatcgacgaccggtagccgtctggtttccaagaacaagtggccgcttgttttccaagcgctgccgccaaagttcctggaaaaaaacctggctaaagcgctggacaaggagtatctgctgccggacatcaacatcgacgagttggaaggcgttgataatggttgtctgattgatgaggccctgttaccgctgatcatcgatgagggcaaacgcaagggtgaaggcaattatcgtccgcgtcacctgcgtgacgaacgtaaagaggagactgtccaggcgtttttggacaaatacggctactgcaatattccggtgggctacgaggtgcatcatattgtgccgctgtctcagggcggtgcggatagtatcaaaaacatgattatgctgtctatcgaacaccacgaaagggttactgaagctcatgcatcatatttcaagtggagaaatacatgatgaagggatatatactgcttgaaaaagtaaatattgaaaacgctaatgcatttaataacattatagtaggaatcccggctatcacctcattcctgggctttgcaagagcgctagagcgcaaactgaacgcgaaagagatcgctatccgtatcaacggtgttggcctggaatttcatgaatatgaattaaaaggttacaagaacaaacgtggtcagtatgtgaccagctgtccgctgccgggttccattccaggtcagaacgagaagaagctggacgctcacatcatgaatcaggcatatattgatctgaatatgagctttttgctggaggtggagggtccgcatgttgatatgtctacgtgcaaatctattaaatcgaccatggaaacgctgcgtattgcgggtggcattattcgcaactacaagaagatccgcttgattgacacccttgccgatatcccgtacggctacttcttgaccctccgtcaggataatctgaatgatgcggcaggcgacgacatgctggataagatgattcatgcgttgcaacaagaggacactttggttccgattgccgttggcttcaaggcgctgagcgaagtcggtcacgtggagggccaacgtgatccggaaaaggaccactgcttcgtggagtccatcttcagcctgggcggttttgaatgtagcaaaatcctggaggacatcaacagctgcctgtggcgctataaaacagaagaagggctatatctttgcaccataatttgactaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccaacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgacacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgactcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgacacgggcaacagctgattgcccttcaccgcctggccctgagagagttgcagcaagcggtccacgctggtttgccccagcaggcgaaaatcctgtttgatggtggttaacggcgggatataacatgagctgtcctcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccg

SEQ ID NO: 79 - DNA, 8513 bp, synthetic construct
  source 1..8513: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatataccatggcagctaataaaaaggcaaccaatgtcacgctaaaatcccgccccgaaaatctatcatttgctaggtgtttaaacacgactgaggcgaaattctggcagaccgattttctgaaacgccatacgttcaaattaccgctgctgattaccgataaggcggtcctcgcgagcaaaggccacgaaatgccgcctgataagctggaaaaagagatcatggacccgaatccgcaaaagtcccagtcttgtaccctgtcgaccgagtgcgatacgctgcgtattgactttggtattaaagtgctgccggttaaagaaagcatgtacagctgcagcgattacaactatcgtaccgcaatttatcagaaaatcgacgaatacatcgcggaggacggcttcttgaccttggcgaagcgctatgtgaataacatcgcgaatgcccgttttttgtggcgtaatcgtaaaggtgctgagatcatcgaaaccattgttaccattgaggacaaggagtacccgagctttaatagcaagtccttcaacctggatactttcgtggaggacaacgccaccatcaacgaaatcgctcagcagattgccgatacgtttgctggcaagcgcgaatacttgaatatttacgtcacctgctttgttaagatcggttgtgcaatggaagtgtatccatcgcaagaaatgacctttgatgacgatgacaagggtaagaaactgttcaagttcgagggtagcgcaggtatgcatagccaaaagatcaacaacgcgctgcgtaccattgacacatggtatccggactacaccacctatgaatttccgattccggtagagaactacggcgcggcgcgttcaatcggcatcccgttcagaccagacaccaaatctttctataaacttatcgaccgcatgattctgaaaaacgaggatctgccgatcgaggacaagcactacgttatggcaattttgattcgaggcggcatgttcagcaagaaacaagaaaaataagcattcaggaggaaacgatatgtttagtcaaattcttatcataaaaccaggcacaggcatatcccccaatattataatctcagaagatatatttccagttctgcatagcttgttcgtggagcacgataaaaagtttggtatcacgttcccggcatactcctttgataaaaagggccatctgggtaatatcattgaagttctgagcgaagataaagaggcgctggcgagcctgtgtctggaggagcacttggcggaagtcaccgactatgtgaaagttaagaaggagatcacgttcaccgacgactacgtgttgtttaaacgcattagagaagagaaccaatatgaaaccactgcccgtcgtatgcgtaaacgcggtcacaccgaactcggccgtccgctggagatgcatattaagaagaagaaccagcaaattttctgccacgcgtacatcaaggtgaaaagcgcatccaccggtcagagctataacatctttcttgctccgaccgacatcaaacacggctcgttttccgcttatggacttcttcgaggtgatactcatgcttagaagcaagcatcaccatcattcaggccatcaccataccggacaccaccatcattcaggcagtcatcaccataccgggggctcaggcgagaacctgtattttcagggttctggtggacttagaaataaaatattggctgccatatcgcaaaaaatccctgaggaacaaaaaataaataagtatattgaagggctgtttcaaagcattgataagaaccaccttgctacgcatgtggccaaattcaccgaaaccaacagcccgggtaatataggtgcgtatgatatcttgtccagcgacatgaactgcggttacctggacaccgcaaatgccggttggaaagaaccggatatcgttaccaacgatgcgaaatacaaacgtcctcagggctttgttgctatggaaatgtccgacggccgtaccgtgatggagcacctacaagaagattctgcagaattaagacacgagatggaagagttgaccgataagtacgacgagatccgtgacggtattttgaacatgccgagcatgcagccgtatcgcacgaaccagtttatcaagcaagttttcttcccggtcggtggtagctatcatctcctcagcattctcccaagcaccgtactgaattacgaggtgagcgaccgcctgtatcgtagcaagatcccgaagattcgcctgcgcttgctgtcgagcaacgcggcatcgacgaccggtagccgtctggtttccaagaacaagtggccgcttgttttccaagcgctgccgccaaagttcctggaaaaaaacctggctaaagcgctggacaaggagtatctgctgccggacatcaacatcgacgagttggaaggcgttgataatggttgtctgattgatgaggccctgttaccgctgatcatcgatgagggcaaacgcaagggtgaaggcaattatcgtccgcgtcacctgcgtgacgaacgtaaagaggagactgtccaggcgtttttggacaaatacggctactgcaatattccggtgggctacgaggtgcatcatattgtgccgctgtctcagggcggtgcggatagtatcaaaaacatgattatgctgtctatcgaacaccacgaaagggttactgaagctcatgcatcatatttcaagtggagaaatacatgatgaagggatatatactgcttgaaaaagtaaatattgaaaacgctaatgcatttaataacattatagtaggaatcccggctatcacctcattcctgggctttgcaagagcgctagagcgcaaactgaacgcgaaagagatcgctatccgtatcaacggtgttggcctggaatttcatgaatatgaattaaaaggttacaagaacaaacgtggtcagtatgtgaccagctgtccgctgccgggttccattccaggtcagaacgagaagaagctggacgctcacatcatgaatcaggcatatattgatctgaatatgagctttttgctggaggtggagggtccgcatgttgatatgtctacgtgcaaatctattaaatcgaccatggaaacgctgcgtattgcgggtggcattattcgcaactacaagaagatccgcttgattgacacccttgccgatatcccgtacggctacttcttgaccctccgtcaggataatctgaatgatgcggcaggcgacgacatgctggataagatgattcatgcgttgcaacaagaggacactttggttccgattgccgttggcttcaaggcgctgagcgaagtcggtcacgtggagggccaacgtgatccggaaaaggaccactgcttcgtggagtccatcttcagcctgggcggttttgaatgtagcaaaatcctggaggacatcaacagctgcctgtggcgctataaaacagaagaagggctatatctttgcaccataatttgactagcataaccccttggggcctctaaacgggtcttgaggggttttttgctgaaaggaggaactatatccggat

SEQ ID NO: 80 - DNA, 3348 bp, synthetic construct
  source 1..3348: mol_type = other DNA
ctcctgcattaggaaattaatacgactcactatagggtgtaccgccggataggcggtttagaaggagaagtcatttaataaggccactgttaaaaagtgtaccgccggataggcggtttagaagctagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcga

SEQ ID NO: 81 - DNA, 9165 bp, synthetic construct
  source 1..9165: mol_type = other DNA
aattgcgttgcgctcactgcccgctttccagtcgggaaacctgtcgtgccagctgcattaatgaatcggccaacgcgcggggagaggcggtttgcgtattgggcgccagggtggtttttcttttcaccagtgacacgggcaacagctgattgcccttcaccgcctggccctgagagagttgcagcaagcggtccacgctggtttgccccagcaggcgaaaatcctgtttgatggtggttaacggcgggatataacatgagctgtcctcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcctcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacggagagcgttcaccgacaaacaacagataaaacgaaaggcccagtctttcgactgagcctttcgttttatttgggcgcgccaaaaaaatcgacctgcaggcgcgccgagctcgaattcggatccggttcacccccacatgcgtggggaatactttttaacagtggccttattaaatgacttctccggttcacccccacatgcgtggggaatacgctagtattatacctaggactgagctagctgtcaacattaattcctaatttttctctcccttatgcgactcctgcattaggaaattttacactttatgcttccggctcgtatgttgggggaattgtgagcggataacaattcccctgtagaaataattttgtttaactttaataaggagatataccatgtattgcgcggcggtttgctttccgcagacaaaatatcagcaaaggggaactgctctaaagaaaccgctcgtcggtctgcgtaagatgggtgtggaagcggctgcatggaacaccctgaaggttactagagatcgccctaagttgacctttccggatctgatcactccgcaaagcaaattcctggataacgacttatggctgaagtacaaagtgccaattgagcagaaagaacacgtcatgtataacctgttgtgcgataactgggttaatgttgtttacttgagcggtaaaccggaccgtatttccttagtccaaaccctgaaggacgcccattgtctgcaattggcgtattctaacccgatggatcgtttcaccgtgttccgcttcctgttggcgttgggttactggtgtttcgcgaacacgaacgtcgagccggagcccgacaaaccgctgccggtgagctggattccgtggctggaggaaaataaagagtacttcgagctgttcggtgatggtaaacgtttcttccaagctgacccgagcagccgtattcgtgcgattactgacctgatccatgaaattccgaccgctcacaacctgtgccattttaagcacgtgaccgactatatcgacggcctgtgtgaagcctgctgcataaaaggtctgttgcgtctgccggtatttaccaccgttggcggtcgtggcattggcgcaggtatcaataacacgccaccgttttatctgctgtggcatgcgaacgacttagcgggcatgttggcgcagaattggcagccttgggacaacatgggcatcccggcgtggctgggctcttttcagaaagagtcgcgggaagttggcctgcttgcgggtatgacctggcttccgcgtaaagtttacctgcacgatccggttccgggtcaggctgcgtgctgcagctgtggtttgccgagtgaagctctggtttactcatgctccattgaggtggaaccagtgccaaagggcttggagtggaaggacccgcacggcgtttacaccgaccagggcaaatccctgcagtccaaaatcaagctgatgagcaatgaccgctataccttcgcggaccgtgactggtattctccgttgttctcgtatcttcacgccgaaggtaatagccgccagggtaagctgtggctggtgggttttgcgagcgataaagcaaaatctatcgacatctgggataagatcatcgagctggagggcaccgacaccaatgacgaactgttggcgcaactcgccaatcgtgcaaccgcgctgaatgcaatgcgtaaaaagccgctacgcggtgattttaaaaagagcgttggcaccccgcagattgcggatatcatcccgcatgcggaaaaccgcatcgccattaacgcaggcaaaatgacggaaaaccgtggttactcttggcaagatgccgatacggagtacggagaactgctgaccaaggtggcatactcgctggagccggcgcaaacggtggatgctcgtctgaagcgcggtaactttatcagccgtaagccgtggccgattatccccgagagcaaaacaaaacccgcagagggggaccagaatgaatagaggaaccgttgacttcattgcaagcttggagaatttaaaagaaggagatctagggatactcagaaagttacgtggtgcgcgtctggacgaaaaacttccgggttttgatctgttcagcgcgctgtggtggccattgcgtcaaaaaaaccagcgcgcaccgaagcgcgaggtggcttggctgattgccaaattgtttgcagaattccgctttgagcaacgtgagggcgcgacgctgccgattctgatgggcggtatctgccgtaagttggaaccgaagaaagaactgccgcgtgtgttggctcgtttcgaccagctggcgagcctggacatcatgcagatggaagagccgctgtccgtgattatgggcatcctgcgcaagcaccagcaggtctgtctggactgggttggtctgaccgatgttttgtcgttctgggaacaagagccggttaaacgtgagtggtctgatagctttatcaaagcgtacaagatcaacaaggaggattcagatgttgattgaaattcatatgattcagaatcactcacccgctaatctaaacagggacgatttaggcgctccaaaaacgtgctatttcggtggcgtgctgcgtagccgcatctcctctcagtgtattaagcgcagcatccgtacgagcaatgacttcaaagcattgctcggcggtgtacgtaccagacgtctggcggatctgattcagcaagaggcgggcgagacagagtgctggaaaaaggctcaagagatcctgaataagtgcggcttcaaaaacaaggacgacaataccaaaatgttggtctttatgagcaaagacaagatcaaagaccttgcgcgtattgttttggacaactcgctgggcctgaccgaagccgcccaacaggtggcgaacgtgatcgctcaggctaccctagctccggacatcgcgctttgcggtcgtatgctggaaccgaatgataaggacaaggacaagaaggtgaagtggtcaaacactaccgttgaggcagcgttgcaagtggcgcatgcaattagcacccatattgcccgtccggaaatcgactacttcgtggccgcggatgatgttccgggtgaagatgcgggtgcgggccacatcggcgaatctatgtttgcaagcgcatgtttttataaatacttcagcatcgattgggaacaactggttaagaacttgaaaggtgataccaacctggcggctcacaccgtcggagcctttctgctggcggcggcgaaaaccaatccgagcggtaaacagaatagctttgccgcgcacaactacccggatggtattctggtcgagttcaagaactccccgatttcctatgcaaatgcttttgttcgcccagttagcgttgttaaagagtcggatttggtcgagcagtccatcggtcagctgagcaactacgtgaacgacatccgcttgggctactatgatgaacagagcccggtgattggtttctggttttccccgaacaaccgttatccgctgggttacaaacacagcaaactggcgagccgtaatattggtaacctgaacgaactcgtgggtgcagttctggattacatcggcggcttcaagtgggaagaagttcagaaaagcaaggcttatattggcggctaacatgagtgcgccacccaacaccttattcttgaggttagaaggggctctacaatcatggggatcgaacgaagcgaagttcgccctgcgccggaccgcagacgcgccaactaaatccggcgttctgggcctgctgtgcgcggctatgggcatcggtcgtgccgaggcggcggactcttggctgccgaaactggctaatctgcgtatgggtgttcgtatcgatcgtccgggcatccgctggtgggattttcacaccgtaggcgcaggccaacgtatgcgtatggcggagctgaaggcgccaaagaagccgagcatggtgggcgcagcgctggcggaaaccctgaccccgagcaaagtcaaaacccgtgcggaaaccttgttgtcccgccgtgagtacctcgcggacgctagcttcctggtggcgttgcaaggtgagccggaacttgtcgcaaagctgtcggcggccttagccaaaccggtttgggcgatttatttgggtcgtaagagctgtccgccatctcgccccgtgtgcgaacatccgccgggcttttataacaccctggaagaagcgctgagcgcagttccgctacaaaaacgttggcataacgagccgctgccgcagattctgccgtgtgttatggactggattccgggttatgatggtgagcacgctccggatgatgcggagatccactacgacttgccggtgagcttccagccgcctcgtcatctgccgcgttttgttattcgtcgggaactggtcgtgggtgaggacgttcaggtttccagagaaactggtacgagcgtctggcgtcctaagggtacgcgtgcggactacaacaacagcgaatacaaaaaggtgcgcgccgagcgcctggtgatggatcacgctgcctgcatggtttgcaaagcaccggcaaccaccgtgcagcacgtgaattatcgtcgcgctggcggtaaagagatcccggaggatttaagagctttgtgccgcctgtgccatgacgcgtgtaccatgttagaatacggctctggtatgaccacgaatcgtatcgacccgtgcgatccgatttggagagaacgtatcctcgctaagcgcaaggagattgttgaatttcgtagccgcggtcagcgtttccgcaaaatgaagccagaggaggaaaacggatgatctacttgtccaggctattgatcgatactggaggtaatccagatagacctaggcccgggcgcaaatggctcgacaacatttataacgtgcatcgtcgtctgagcatggcatttccgagcggtctgcgccgtgaacaggacccgcattttctgaagccgttttctccgaatgacttccagaaaacgccattcttgtttcgtgtcgacaataatatcgacggcaacgataaacgtgcgattatcatcgtgcaaagcgtcctggagcccgactgggattactgctttcaaaacgccttagacttcttggcggcgccaccggaaaccaaagagtacaacccggaatttaaagcgggtcagttgctccggttccgcctgcgtgttaatgcatccgtgcgtagacacatcccggagatggttcagcaggatggccagaccattgaaactggtaagatcctgcacaagcgcgtgagcctgacctgggacgctagctcgaccccggatcaggctttagctgattggctggcggcgaagagcccgaagttgggcttcacgctgcagcgttgtgaactgctgcaactgggttgggtttatggtagcaaaccggaaccgaagaacgtgaaagttaaagaacaaggccaaggctattggcgtgagcacaaatacaacccgctgcgcttccgtgccgcattgcttgagggcgttctggaggttgatgatccgaagctgttcctgaaaaccctgagcagcggtattggtaaggcgaagagttttggtttcggcctgctgtccgttctgccaatcaggaatgatggataaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaaccaggcgtttaagggcaccaataactgccttaaaaaaattacgccccgccctgccactcatcgcagtactgttgtaattcattaagcattctgccaacatggaagccatcacagacggcatgatgaacctgaatcgccagcggcatcagcaccttgtcgccttgcgtataatatttgcccatagtgaaaacgggggcgaagaagttgtccatattggccacgtttaaatcaaaactggtgaaactcacccagggattggctgacacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaactccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgactcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttcccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacatt

SEQ ID NO: 82 - DNA, 10377 bp, synthetic construct
  source 1..10377: mol_type = other DNA
tggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatataccatgtattgcgcggcggtttgctttccgcagacaaaatatcagcaaaggggaactgctctaaagaaaccgctcgtcggtctgcgtaagatgggtgtggaagcggctgcatggaacaccctgaaggttactagagatcgccctaagttgacctttccggatctgatcactccgcaaagcaaattcctggataacgacttatggctgaagtacaaagtgccaattgagcagaaagaacacgtcatgtataacctgttgtgcgataactgggttaatgttgtttacttgagcggtaaaccggaccgtatttccttagtccaaaccctgaaggacgcccattgtctgcaattggcgtattctaacccgatggatcgtttcaccgtgttccgcttcctgttggcgttgggttactggtgtttcgcgaacacgaacgtcgagccggagcccgacaaaccgctgccggtgagctggattccgtggctggaggaaaataaagagtacttcgagctgttcggtgatggtaaacgtttcttccaagctgacccgagcagccgtattcgtgcgattactgacctgatccatgaaattccgaccgctcacaacctgtgccattttaagcacgtgaccgactatatcgacggcctgtgtgaagcctgctgcataaaaggtctgttgcgtctgccggtatttaccaccgttggcggtcgtggcattggcgcaggtatcaataacacgccaccgttttatctgctgtggcatgcgaacgacttagcgggcatgttggcgcagaattggcagccttgggacaacatgggcatcccggcgtggctgggctcttttcagaaagagtcgcgggaagttggcctgcttgcgggtatgacctggcttccgcgtaaagtttacctgcacgatccggttccgggtcaggctgcgtgctgcagctgtggtttgccgagtgaagctctggtttactcatgctccattgaggtggaaccagtgccaaagggcttggagtggaaggacccgcacggcgtttacaccgaccagggcaaatccctgcagtccaaaatcaagctgatgagcaatgaccgctataccttcgcggaccgtgactggtattctccgttgttctcgtatcttcacgccgaaggtaatagccgccagggtaagctgtggctggtgggttttgcgagcgataaagcaaaatctatcgacatctgggataagatcatcgagctggagggcaccgacaccaatgacgaactgttggcgcaactcgccaatcgtgcaaccgcgctgaatgcaatgcgtaaaaagccgctacgcggtgattttaaaaagagcgttggcaccccgcagattgcggatatcatcccgcatgcggaaaaccgcatcgccattaacgcaggcaaaatgacggaaaaccgtggttactcttggcaagatgccgatacggagtacggagaactgctgaccaaggtggcatactcgctggagccggcgcaaacggtggatgctcgtctgaagcgcggtaactttatcagccgtaagccgtggccgattatccccgagagcaaaacaaaacccgcagagggggaccagaatgaatagaggaagcaagcatcaccatcattcaggccatcaccataccggacaccaccatcattcaggcagtcatcaccataccgggggctcaggcgagaacctgtattttcagggttctggtggaaccgttgacttcattgcaagcttggagaatttaaaagaaggagatctagggatactcagaaagttacgtggtgcgcgtctggacgaaaaacttccgggttttgatctgttcagcgcgctgtggtggccattgcgtcaaaaaaaccagcgcgcaccgaagcgcgaggtggcttggctgattgccaaattgtttgcagaattccgctttgagcaacgtgagggcgcgacgctgccgattctgatgggcggtatctgccgtaagttggaaccgaagaaagaactgccgcgtgtgttggctcgtttcgaccagctggcgagcctggacatcatgcagatggaagagccgctgtccgtgattatgggcatcctgcgcaagcaccagcaggtctgtctggactgggttggtctgaccgatgttttgtcgttctgggaacaagagccggttaaacgtgagtggtctgatagctttatcaaagcgtacaagatcaacaaggaggattcagatgttgattgaaattcatatgattcagaatcactcacccgctaatctaaacagggacgatttaggcgctccaaaaacgtgctatttcggtggcgtgctgcgtagccgcatctcctctcagtgtattaagcgcagcatccgtacgagcaatgacttcaaagcattgctcggcggtgtacgtaccagacgtctggcggatctgattcagcaagaggcgggcgagacagagtgctggaaaaaggctcaagagatcctgaataagtgcggcttcaaaaacaaggacgacaataccaaaatgttggtctttatgagcaaagacaagatcaaagaccttgcgcgtattgttttggacaactcgctgggcctgaccgaagccgcccaacaggtggcgaacgtgatcgctcaggctaccctagctccggacatcgcgctttgcggtcgtatgctggaaccgaatgataaggacaaggacaagaaggtgaagtggtcaaacactaccgttgaggcagcgttgcaagtggcgcatgcaattagcacccatattgcccgtccggaaatcgactacttcgtggccgcggatgatgttccgggtgaagatgcgggtgcgggccacatcggcgaatctatgtttgcaagcgcatgtttttataaatacttcagcatcgattgggaacaactggttaagaacttgaaaggtgataccaacctggcggctcacaccgtcggagcctttctgctggcggcggcgaaaaccaatccgagcggtaaacagaatagctttgccgcgcacaactacccggatggtattctggtcgagttcaagaactccccgatttcctatgcaaatgcttttgttcgcccagttagcgttgttaaagagtcggatttggtcgagcagtccatcggtcagctgagcaactacgtgaacgacatccgcttgggctactatgatgaacagagcccggtgattggtttctggttttccccgaacaaccgttatccgctgggttacaaacacagcaaactggcgagccgtaatattggtaacctgaacgaactcgtgggtgcagttctggattacatcggcggcttcaagtgggaagaagttcagaaaagcaaggcttatattggcggctaacatgagtgcgccacccaacaccttattcttgaggttagaaggggctctacaatcatggggatcgaacgaagcgaagttcgccctgcgccggaccgcagacgcgccaactaaatccggcgttctgggcctgctgtgcgcggctatgggcatcggtcgtgccgaggcggcggactcttggctgccgaaactggctaatctgcgtatgggtgttcgtatcgatcgtccgggcatccgctggtgggattttcacaccgtaggcgcaggccaacgtatgcgtatggcggagctgaaggcgccaaagaagccgagcatggtgggcgcagcgctggcggaaaccctgaccccgagcaaagtcaaaacccgtgcggaaaccttgttgtcccgccgtgagtacctcgcggacgctagcttcctggtggcgttgcaaggtgagccggaacttgtcgcaaagctgtcggcggccttagccaaaccggtttgggcgatttatttgggtcgtaagagctgtccgccatctcgccccgtgtgcgaacatccgccgggcttttataacaccctggaagaagcgctgagcgcagttccgctacaaaaacgttggcataacgagccgctgccgcagattctgccgtgtgttatggactggattccgggttatgatggtgagcacgctccggatgatgcggagatccactacgacttgccggtgagcttccagccgcctcgtcatctgccgcgttttgttattcgtcgggaactggtcgtgggtgaggacgttcaggtttccagagaaactggtacgagcgtctggcgtcctaagggtacgcgtgcggactacaacaacagcgaatacaaaaaggtgcgcgccgagcgcctggtgatggatcacgctgcctgcatggtttgcaaagcaccggcaaccaccgtgcagcacgtgaattatcgtcgcgctggcggtaaagagatcccggaggatttaagagctttgtgccgcctgtgccatgacgcgtgtaccatgttagaatacggctctggtatgaccacgaatcgtatcgacccgtgcgatccgatttggagagaacgtatcctcgctaagcgcaaggagattgttgaatttcgtagccgcggtcagcgtttccgcaaaatgaagccagaggaggaaaacggatgatctacttgtccaggctattgatcgatactggaggtaatccagatagacctaggcccgggcgcaaatggctcgacaacatttataacgtgcatcgtcgtctgagcatggcatttccgagcggtctgcgccgtgaacaggacccgcattttctgaagccgttttctccgaatgacttccagaaaacgccattcttgtttcgtgtcgacaataatatcgacggcaacgataaacgtgcgattatcatcgtgcaaagcgtcctggagcccgactgggattactgctttcaaaacgccttagacttcttggcggcgccaccggaaaccaaagagtacaacccggaatttaaagcgggtcagttgctccggttccgcctgcgtgttaatgcatccgtgcgtagacacatcccggagatggttcagcaggatggccagaccattgaaactggtaagatcctgcacaagcgcgtgagcctgacctgggacgctagctcgaccccggatcaggctttagctgattggctggcggcgaagagcccgaagttgggcttcacgctgcagcgttgtgaactgctgcaactgggttgggtttatggtagcaaaccggaaccgaagaacgtgaaagttaaagaacaaggccaaggctattggcgtgagcacaaatacaacccgctgcgcttccgtgccgcattgcttgagggcgttctggaggttgatgatccgaagctgttcctgaaaaccctgagcagcggtattggtaaggcgaagagttttggtttcggcctgctgtccgttctgccaatcaggaatgatggataactagcataaccccttggggcctctaaacgggtcttgaggggttttttgctgaaaggaggaactatatccggat

SEQ ID NO: 83 - DNA, 3350 bp, synthetic construct
  source 1..3350: mol_type = other DNA
ctcctgcattaggaaattaatacgactcactatagggtattccccacgcatgtgggggtgaaccggagaagtcatttaataaggccactgttaaaaagtattccccacgcatgtgggggtgaaccgctagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgacaagctgacgaccgggtctccgcaagtggcacttttcggggaaatgtgcgcggaacccctatttgtttatttttctaaatacattcaaatatgtatccgctcatgaattaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcggtcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggcatgctagcgcagaaacgtcctagaagatgccaggaggatacttagcagagagacaataaggccggagcgaagccgtttttccataggctccgcccccctgacgaacatcacgaaatctgacgctcaaatcagtggtggcgaaacccgacaggactataaagataccaggcgtttccccctgatggctccctcttgcgctctcctgttcccgtcctgcggcgtccgtgttgtggtggaggctttacccaaatcaccacgtcccgttccgtgtagacagttcgctccaagctgggctgtgtgcaagaaccccccgttcagcccgactgctgcgccttatccggtaactatcatcttgagtccaacccggaaagacacgacaaaacgccactggcagcagccattggtaactgagaattagtggatttagatatcgagagtcttgaagtggtggcctaacagaggctacactgaaaggacagtatttggtatctgcgctccactaaagccagttaccaggttaagcagttccccaactgacttaaccttcgatcaaaccgcctccccaggcggttttttcgtttacagagcaggagattacgacgatcgtaaaaggatctcaagaagatcctttacggattcccgacaccatcactctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcga

SEQ ID NO: 84 - DNA, 6454 bp, synthetic construct
  source 1..6454: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatccatggcagctaataaaaaggcaaccaatgtcacgctaaaatcccgccccgaaaatctatcatttgctaggtgtttaaacacgactgaggcgaaattctggcagaccgattttctgaaacgccatacgttcaaattaccgctgctgattaccgataaggcggtcctcgcgagcaaaggccacgaaatgccgcctgataagctggaaaaagagatcatggacccgaatccgcaaaagtcccagtcttgtaccctgtcgaccgagtgcgatacgctgcgtattgactttggtattaaagtgctgccggttaaagaaagcatgtacagctgcagcgattacaactatcgtaccgcaatttatcagaaaatcgacgaatacatcgcggaggacggcttcttgaccttggcgaagcgctatgtgaataacatcgcgaatgcccgttttttgtggcgtaatcgtaaaggtgctgagatcatcgaaaccattgttaccattgaggacaaggagtacccgagctttaatagcaagtccttcaacctggatactttcgtggaggacaacgccaccatcaacgaaatcgctcagcagattgccgatacgtttgctggcaagcgcgaatacttgaatatttacgtcacctgctttgttaagatcggttgtgcaatggaagtgtatccatcgcaagaaatgacctttgatgacgatgacaagggtaagaaactgttcaagttcgagggtagcgcaggtatgcatagccaaaagatcaacaacgcgctgcgtaccattgacacatggtatccggactacaccacctatgaatttccgattccggtagagaactacggcgcggcgcgttcaatcggcatcccgttcagaccagacaccaaatctttctataaacttatcgaccgcatgattctgaaaaacgaggatctgccgatcgaggacaagcactacgttatggcaattttgattcgaggcggcatgttcagcaagaaacaagaaaaataaggatcctgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 85 - DNA, 5983 bp, synthetic construct
  source 1..5983: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatccatgtttagtcaaattcttatcataaaaccaggcacaggcatatcccccaatattataatctcagaagatatatttccagttctgcatagcttgttcgtggagcacgataaaaagtttggtatcacgttcccggcatactcctttgataaaaagggccatctgggtaatatcattgaagttctgagcgaagataaagaggcgctggcgagcctgtgtctggaggagcacttggcggaagtcaccgactatgtgaaagttaagaaggagatcacgttcaccgacgactacgtgttgtttaaacgcattagagaagagaaccaatatgaaaccactgcccgtcgtatgcgtaaacgcggtcacaccgaactcggccgtccgctggagatgcatattaagaagaagaaccagcaaattttctgccacgcgtacatcaaggtgaaaagcgcatccaccggtcagagctataacatctttcttgctccgaccgacatcaaacacggctcgttttccgcttatggacttcttcgaggtgatactcatgcctgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 86 - DNA, 6481 bp, synthetic construct
  source 1..6481: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatccatgcttagaaataaaatattggctgccatatcgcaaaaaatccctgaggaacaaaaaataaataagtatattgaagggctgtttcaaagcattgataagaaccaccttgctacgcatgtggccaaattcaccgaaaccaacagcccgggtaatataggtgcgtatgatatcttgtccagcgacatgaactgcggttacctggacaccgcaaatgccggttggaaagaaccggatatcgttaccaacgatgcgaaatacaaacgtcctcagggctttgttgctatggaaatgtccgacggccgtaccgtgatggagcacctacaagaagattctgcagaattaagacacgagatggaagagttgaccgataagtacgacgagatccgtgacggtattttgaacatgccgagcatgcagccgtatcgcacgaaccagtttatcaagcaagttttcttcccggtcggtggtagctatcatctcctcagcattctcccaagcaccgtactgaattacgaggtgagcgaccgcctgtatcgtagcaagatcccgaagattcgcctgcgcttgctgtcgagcaacgcggcatcgacgaccggtagccgtctggtttccaagaacaagtggccgcttgttttccaagcgctgccgccaaagttcctggaaaaaaacctggctaaagcgctggacaaggagtatctgctgccggacatcaacatcgacgagttggaaggcgttgataatggttgtctgattgatgaggccctgttaccgctgatcatcgatgagggcaaacgcaagggtgaaggcaattatcgtccgcgtcacctgcgtgacgaacgtaaagaggagactgtccaggcgtttttggacaaatacggctactgcaatattccggtgggctacgaggtgcatcatattgtgccgctgtctcagggcggtgcggatagtatcaaaaacatgattatgctgtctatcgaacaccacgaaagggttactgaagctcatgcatcatatttcaagtggagaaatacatgaggatcctgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 87 - DNA, 6211 bp, synthetic construct
  source 1..6211: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatccatgaagggatatatactgcttgaaaaagtaaatattgaaaacgctaatgcatttaataacattatagtaggaatcccggctatcacctcattcctgggctttgcaagagcgctagagcgcaaactgaacgcgaaagagatcgctatccgtatcaacggtgttggcctggaatttcatgaatatgaattaaaaggttacaagaacaaacgtggtcagtatgtgaccagctgtccgctgccgggttccattccaggtcagaacgagaagaagctggacgctcacatcatgaatcaggcatatattgatctgaatatgagctttttgctggaggtggagggtccgcatgttgatatgtctacgtgcaaatctattaaatcgaccatggaaacgctgcgtattgcgggtggcattattcgcaactacaagaagatccgcttgattgacacccttgccgatatcccgtacggctacttcttgaccctccgtcaggataatctgaatgatgcggcaggcgacgacatgctggataagatgattcatgcgttgcaacaagaggacactttggttccgattgccgttggcttcaaggcgctgagcgaagtcggtcacgtggagggccaacgtgatccggaaaaggaccactgcttcgtggagtccatcttcagcctgggcggttttgaatgtagcaaaatcctggaggacatcaacagctgcctgtggcgctataaaacagaagaagggctatatctttgcaccataatttgaggatcctgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 88 - DNA, 2982 bp, synthetic construct
  source 1..2982: mol_type = other DNA
gttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgtaccgccggataggcggtttagaagatgtcttcctgggacgaagacaagtgtaccgccggataggcggtttagaagttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcct

SEQ ID NO: 89 - DNA, 7054 bp, synthetic construct
  source 1..7054: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatctatgtattgcgcggcggtttgctttccgcagacaaaatatcagcaaaggggaactgctctaaagaaaccgctcgtcggtctgcgtaagatgggtgtggaagcggctgcatggaacaccctgaaggttactagagatcgccctaagttgacctttccggatctgatcactccgcaaagcaaattcctggataacgacttatggctgaagtacaaagtgccaattgagcagaaagaacacgtcatgtataacctgttgtgcgataactgggttaatgttgtttacttgagcggtaaaccggaccgtatttccttagtccaaaccctgaaggacgcccattgtctgcaattggcgtattctaacccgatggatcgtttcaccgtgttccgcttcctgttggcgttgggttactggtgtttcgcgaacacgaacgtcgagccggagcccgacaaaccgctgccggtgagctggattccgtggctggaggaaaataaagagtacttcgagctgttcggtgatggtaaacgtttcttccaagctgacccgagcagccgtattcgtgcgattactgacctgatccatgaaattccgaccgctcacaacctgtgccattttaagcacgtgaccgactatatcgacggcctgtgtgaagcctgctgcataaaaggtctgttgcgtctgccggtatttaccaccgttggcggtcgtggcattggcgcaggtatcaataacacgccaccgttttatctgctgtggcatgcgaacgacttagcgggcatgttggcgcagaattggcagccttgggacaacatgggcatcccggcgtggctgggctcttttcagaaagagtcgcgggaagttggcctgcttgcgggtatgacctggcttccgcgtaaagtttacctgcacgatccggttccgggtcaggctgcgtgctgcagctgtggtttgccgagtgaagctctggtttactcatgctccattgaggtggaaccagtgccaaagggcttggagtggaaggacccgcacggcgtttacaccgaccagggcaaatccctgcagtccaaaatcaagctgatgagcaatgaccgctataccttcgcggaccgtgactggtattctccgttgttctcgtatcttcacgccgaaggtaatagccgccagggtaagctgtggctggtgggttttgcgagcgataaagcaaaatctatcgacatctgggataagatcatcgagctggagggcaccgacaccaatgacgaactgttggcgcaactcgccaatcgtgcaaccgcgctgaatgcaatgcgtaaaaagccgctacgcggtgattttaaaaagagcgttggcaccccgcagattgcggatatcatcccgcatgcggaaaaccgcatcgccattaacgcaggcaaaatgacggaaaaccgtggttactcttggcaagatgccgatacggagtacggagaactgctgaccaaggtggcatactcgctggagccggcgcaaacggtggatgctcgtctgaagcgcggtaactttatcagccgtaagccgtggccgattatccccgagagcaaaacaaaacccgcagagggggaccagaatgaatagggatcttgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 90 - DNA, 5971 bp, synthetic construct
  source 1..5971: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatctatgaatagaggaaccgttgacttcattgcaagcttggagaatttaaaagaaggagatctagggatactcagaaagttacgtggtgcgcgtctggacgaaaaacttccgggttttgatctgttcagcgcgctgtggtggccattgcgtcaaaaaaaccagcgcgcaccgaagcgcgaggtggcttggctgattgccaaattgtttgcagaattccgctttgagcaacgtgagggcgcgacgctgccgattctgatgggcggtatctgccgtaagttggaaccgaagaaagaactgccgcgtgtgttggctcgtttcgaccagctggcgagcctggacatcatgcagatggaagagccgctgtccgtgattatgggcatcctgcgcaagcaccagcaggtctgtctggactgggttggtctgaccgatgttttgtcgttctgggaacaagagccggttaaacgtgagtggtctgatagctttatcaaagcgtacaagatcaacaaggaggattcagatgttgattgaggatcttgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 91 - DNA, 6583 bp, synthetic construct
  source 1..6583: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatctatgttgattgaaattcatatgattcagaatcactcacccgctaatctaaacagggacgatttaggcgctccaaaaacgtgctatttcggtggcgtgctgcgtagccgcatctcctctcagtgtattaagcgcagcatccgtacgagcaatgacttcaaagcattgctcggcggtgtacgtaccagacgtctggcggatctgattcagcaagaggcgggcgagacagagtgctggaaaaaggctcaagagatcctgaataagtgcggcttcaaaaacaaggacgacaataccaaaatgttggtctttatgagcaaagacaagatcaaagaccttgcgcgtattgttttggacaactcgctgggcctgaccgaagccgcccaacaggtggcgaacgtgatcgctcaggctaccctagctccggacatcgcgctttgcggtcgtatgctggaaccgaatgataaggacaaggacaagaaggtgaagtggtcaaacactaccgttgaggcagcgttgcaagtggcgcatgcaattagcacccatattgcccgtccggaaatcgactacttcgtggccgcggatgatgttccgggtgaagatgcgggtgcgggccacatcggcgaatctatgtttgcaagcgcatgtttttataaatacttcagcatcgattgggaacaactggttaagaacttgaaaggtgataccaacctggcggctcacaccgtcggagcctttctgctggcggcggcgaaaaccaatccgagcggtaaacagaatagctttgccgcgcacaactacccggatggtattctggtcgagttcaagaactccccgatttcctatgcaaatgcttttgttcgcccagttagcgttgttaaagagtcggatttggtcgagcagtccatcggtcagctgagcaactacgtgaacgacatccgcttgggctactatgatgaacagagcccggtgattggtttctggttttccccgaacaaccgttatccgctgggttacaaacacagcaaactggcgagccgtaatattggtaacctgaacgaactcgtgggtgcagttctggattacatcggcggcttcaagtgggaagaagttcagaaaagcaaggcttatattggcggctaaggatcttgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 92 - DNA, 6613 bp, synthetic construct
  source 1..6613: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatctatgagtgcgccacccaacaccttattcttgaggttagaaggggctctacaatcatggggatcgaacgaagcgaagttcgccctgcgccggaccgcagacgcgccaactaaatccggcgttctgggcctgctgtgcgcggctatgggcatcggtcgtgccgaggcggcggactcttggctgccgaaactggctaatctgcgtatgggtgttcgtatcgatcgtccgggcatccgctggtgggattttcacaccgtaggcgcaggccaacgtatgcgtatggcggagctgaaggcgccaaagaagccgagcatggtgggcgcagcgctggcggaaaccctgaccccgagcaaagtcaaaacccgtgcggaaaccttgttgtcccgccgtgagtacctcgcggacgctagcttcctggtggcgttgcaaggtgagccggaacttgtcgcaaagctgtcggcggccttagccaaaccggtttgggcgatttatttgggtcgtaagagctgtccgccatctcgccccgtgtgcgaacatccgccgggcttttataacaccctggaagaagcgctgagcgcagttccgctacaaaaacgttggcataacgagccgctgccgcagattctgccgtgtgttatggactggattccgggttatgatggtgagcacgctccggatgatgcggagatccactacgacttgccggtgagcttccagccgcctcgtcatctgccgcgttttgttattcgtcgggaactggtcgtgggtgaggacgttcaggtttccagagaaactggtacgagcgtctggcgtcctaagggtacgcgtgcggactacaacaacagcgaatacaaaaaggtgcgcgccgagcgcctggtgatggatcacgctgcctgcatggtttgcaaagcaccggcaaccaccgtgcagcacgtgaattatcgtcgcgctggcggtaaagagatcccggaggatttaagagctttgtgccgcctgtgccatgacgcgtgtaccatgttagaatacggctctggtatgaccacgaatcgtatcgacccgtgcgatccgatttggagagaacgtatcctcgctaagcgcaaggagattgttgaatttcgtagccgcggtcagcgtttccgcaaaatgaagccagaggaggaaaacggatgaggatcttgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 93 - DNA, 6265 bp, synthetic construct
  source 1..6265: mol_type = other DNA
aatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttgccaccatggactataaggatgatgacgacaagatggccggatctccaaaaaagaaacggaaggtgggatctatgatctacttgtccaggctattgatcgatactggaggtaatccagatagacctaggcccgggcgcaaatggctcgacaacatttataacgtgcatcgtcgtctgagcatggcatttccgagcggtctgcgccgtgaacaggacccgcattttctgaagccgttttctccgaatgacttccagaaaacgccattcttgtttcgtgtcgacaataatatcgacggcaacgataaacgtgcgattatcatcgtgcaaagcgtcctggagcccgactgggattactgctttcaaaacgccttagacttcttggcggcgccaccggaaaccaaagagtacaacccggaatttaaagcgggtcagttgctccggttccgcctgcgtgttaatgcatccgtgcgtagacacatcccggagatggttcagcaggatggccagaccattgaaactggtaagatcctgcacaagcgcgtgagcctgacctgggacgctagctcgaccccggatcaggctttagctgattggctggcggcgaagagcccgaagttgggcttcacgctgcagcgttgtgaactgctgcaactgggttgggtttatggtagcaaaccggaaccgaagaacgtgaaagttaaagaacaaggccaaggctattggcgtgagcacaaatacaacccgctgcgcttccgtgccgcattgcttgagggcgttctggaggttgatgatccgaagctgttcctgaaaaccctgagcagcggtattggtaaggcgaagagttttggtttcggcctgctgtccgttctgccaatcaggaatgatggataaggatcttgaggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgctt

SEQ ID NO: 94 - DNA, 2985 bp, synthetic construct
  source 1..2985: mol_type = other DNA
gttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggtattccccacgcatgtgggggtgaaccgatgtcttcctgggacgaagacaagtattccccacgcatgtgggggtgaaccgttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcct

SEQ ID NO: 95 - protein, 38 aa, synthetic construct
QLGWVYGSKPEPKNVKVKEQGQGYWREHKYNPLRFRAA

SEQ ID NO: 96 - protein, 23 aa, synthetic construct
SVLEPDWDYCFQNALDFLAAPPE

SEQ ID NO: 97 - protein, 58 aa, synthetic construct
LIDTGGNPDRPRPGRKWLDNIYNVHRRLSMAFPSGLRREQDPHFLKPFSPNDFQKTPF

SEQ ID NO: 98 - protein, 7 aa, synthetic construct
SLDIMQM

SEQ ID NO: 99 - protein, 6 aa, synthetic construct
SFWEQE

SEQ ID NO: 100 - protein, 31 aa, synthetic construct
RLDEKLPGFDLFSALWWPLRQKNQRAPKREV

SEQ ID NO: 101 - protein, 9 aa, synthetic construct
TSVWRPKGT

SEQ ID NO: 102 - protein, 8 aa, synthetic construct
TRADYNNS

SEQ ID NO: 103 - protein, 27 aa, synthetic construct
NSEYKKVRAERLVMDHAACMVCKAPAT

SEQ ID NO: 104 - protein, 13 aa, synthetic construct
DHAACMVCKAPAT

SEQ ID NO: 105 - protein, 15 aa, synthetic construct
CRLCHDACTMLEYGS

SEQ ID NO: 106 - protein, 21 aa, synthetic construct
RLCHDACTMLEYGSGMTTNRI

SEQ ID NO: 107 - protein, 38 aa, synthetic construct
DPCDPIWRERILAKRKEIVEFRSRGQRFRKMKPEEENG

SEQ ID NO: 108 - protein, 34 aa, synthetic construct
PIWRERILAKRKEIVEFRSRGQRFRKMKPEEENG

SEQ ID NO: 109 - protein, 388 aa, synthetic construct
MSAPPNTLFLRLEGALQSWGSNEAKFALRRTADAPTKSGVLGLLCAAMGIGRAEAADSWLPKLANLRMGVRIDRPGIRWWDFHTVGAGQRMRMAELKAPKKPSMVGAALAETLTPSKVKTRAETLLSRREYLADASFLVALQGEPELVAKLSAALAKPVWAIYLGRKSCPPSRPVCEHPPGFYNTLEEALSAVPLQKRWHNEPLPQILPCVMDWIPGYDGEHAPDDAEIHYDLPVSFQPPRHLPRFVIRRELVVGEDVQVSRETGTSVWRPKGTRADYNNSEYKKVRAERLVMDHAACMVCKAPATTVQHVNYRRAGGKEIPEDLRALCRLCHDACTMLEYGSGMTTNRIDPCDPIWRERILAKRKEIVEFRSRGQRFRKMKPEEENG

SEQ ID NO: 110 - protein, 272 aa, synthetic construct
MIYLSRLLIDTGGNPDRPRPGRKWLDNIYNVHRRLSMAFPSGLRREQDPHFLKPFSPNDFQKTPFLFRVDNNIDGNDKRAIIIVQSVLEPDWDYCFQNALDFLAAPPETKEYNPEFKAGQLLRFRLRVNASVRRHIPEMVQQDGQTIETGKILHKRVSLTWDASSTPDQALADWLAAKSPKLGFTLQRCELLQLGWVYGSKPEPKNVKVKEQGQGYWREHKYNPLRFRAALLEGVLEVDDPKLFLKTLSSGIGKAKSFGFGLLSVLPIRNDG

SEQ ID NO: 111 - protein, 174 aa, synthetic construct
MNRGTVDFIASLENLKEGDLGILRKLRGARLDEKLPGFDLFSALWWPLRQKNQRAPKREVAWLIAKLFAEFRFEQREGATLPILMGGICRKLEPKKELPRVLARFDQLASLDIMQMEEPLSVIMGILRKHQQVCLDWVGLTDVLSFWEQEPVKREWSDSFIKAYKINKEDSDVD

(19) United States
(12) Patent Application Publication
(10) Pub. No.: US 2025/0223577 A1
(43) Pub. Date: Jul. 10, 2025

(54) HYBRID CRISPR-CAS SYSTEMS AND METHODS OF USE THEREOF

(71) Applicants: The Broad Institute, Inc., Cambridge, MA (US); Massachusetts Institute of Technology, Cambridge, MA (US)

(72) Inventors: Feng Zhang, Cambridge, MA (US); Han Altae-Tran, Cambridge, MA (US); Soumya Kannan, Cambridge, MA (US)

(21) Appl. No.: 19/016,260

(22) Filed: Jan. 10, 2025

Related U.S. Application Data
(63) Continuation of application No. PCT/US2023/070150, filed on Jul. 13, 2023.
(60) Provisional application No. 63/368,329, filed on Jul. 13, 2022.

(51) Int. Cl. C12N 9/22; C12N 9/12; C12N 15/11; C12N 15/115; C12N 15/90

(52) U.S. Cl. CPC C12N 9/22; C12N 9/1276; C12N 15/11; C12N 15/115; C12N 15/907; C12Y 207/07049; C12N 2310/16; C12N 2310/20

(57) ABSTRACT

Engineered or non-naturally occurring systems and compositions comprising novel Cas5-HNH and Cas8-HNH polypeptides are detailed herein. Also provided are methods and applications for the novel Cas5-HNH and Cas8-HNH polypeptides for reprogrammable targeting nucleic acid and polynucleotide components.

### CROSS-REFERENCE TO RELATED APPLICATIONS

This application is a continuation application of PCT/US2023/070150, filed Jul. 13, 2023, which claims the benefit of U.S. Provisional Application No. 63/368,329, filed Jul. 13, 2022. The entire contents of the above-identified applications are hereby fully incorporated herein by reference.

### STATEMENT REGARDING FEDERALLY SPONSORED RESEARCH

This invention was made with government support under Grant No. (s) HL141201 and HG009761 awarded by the National Institutes of Health. The government has certain rights in the invention.

### REFERENCE TO AN ELECTRONIC SEQUENCE LISTING

The contents of the electronic sequence listing (“BROD-5640US_ST26_revised”; Size is 441,989 bytes and it was created on Feb. 3, 2025) is herein incorporated by reference in its entirety.

### TECHNICAL FIELD

The subject matter disclosed herein is generally directed to systems, methods and compositions used for targeted gene modification and nucleic acid editing utilizing systems comprising Cas5-HNH and Cas8-HNH polypeptides. In particular, the present disclosure provides DNA or RNA-targeting compositions comprising novel DNA or RNA-targeting nucleases and at least one targeting nucleic acid component.

### BACKGROUND

The exploration of CRISPR-Cas systems of bacterial adaptive immunity to bacteriophage (viral) infection has shown these systems comprise extreme diversity of protein composition and genomic architecture. While there are genome-editing techniques available for producing targeted genome perturbations, there remains a pressing need for new and alternative genome engineering technologies that employ robust novel strategies and molecular mechanisms and are affordable, easy to set up, scalable, and amenable to targeting multiple positions within the genome. Among the systems identified, it has been shown that size sometimes is a constraint to full deployment of these systems and that smaller, robust systems might confer some advantages. Thus, there exists a pressing need for alternative and robust systems of smaller size for targeting nucleic acids or polynucleotides. These additional desirable tools in genome engineering and biotechnology would further advance the art.

Citation or identification of any document in this application is not an admission that such a document is available as prior art to the present invention.

### SUMMARY

In one aspect, as described herein, a non-naturally occurring, engineered composition comprising a) a hybrid CRISPR-Cas polypeptide comprising a Cas5 or Cas8 polypeptide, the Cas5 or Cas8 polypeptide comprising an HNH domain; and b) a guide RNA molecule comprising a reprogrammable spacer sequence, the guide RNA molecule capable of forming a complex with the hybrid CRISPR-Cas polypeptide and directing the polypeptide to a target polynucleotide. In one example embodiment, wherein the HNH domain is located in or at the C-terminus of the Cas5 or Cas8 polypeptide. In an example embodiment, wherein the composition comprises a Cas5 polypeptide, the composition further comprises one or more of a Cas8e, a Cas11, a Cas7 and/or a Cas6 polypeptide or, wherein the composition comprises a Cas8 polypeptide, further comprising one or more of a Cas5, Cas6, Cas7, Cas11, and/or Cas12. In an example embodiment, the hybrid CRISPR-Cas polypeptide comprises the amino acids corresponding to the about 1,000 to about 1,200 nucleic acids encoding the protein. In an example embodiment, the reprogrammable spacer sequence comprises a spacer of 10 nucleotides to 40 nucleotides in length, preferably 15 to 35 nt in length.

In an example embodiment, the target sequence comprises a protospacer adjacent motif (PAM) sequence 5′ of the target polynucleotide. In an example embodiment, the PAM sequence comprises Ax, Tx, Gx, or xC wherein x consist of any nucleotide. In an example embodiment, the target polynucleotide is DNA. In an example embodiment, the guide RNA further comprises an aptamer. In an example embodiment, the guide RNA molecule further comprises an extension to add an RNA template. In example embodiment, the HNH domain of the hybrid CRISPR-Cas protein is catalytically inactive.

In an example embodiment, the composition further comprises a functional domain associated with the hybrid CRISPR-Cas protein. In an example embodiment, the functional domain has transposase activity, methylase activity, demethylase activity, translation activation activity, translation repression activity, transcription activation activity, transcription repression activity, translation release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

In an example embodiment, the composition comprises a serine or tyrosine recombinase. In an example embodiment, the composition comprises a nucleotide deaminase. In an example embodiment, the composition comprises a reverse transcriptase and further comprises a donor polynucleotide sequence, optionally wherein the donor polynucleotide further comprises a homology region to the target sequence on the 5′ end of the donor construct, the 3′ end of the donor construct, or both. In an example embodiment, the composition comprises a non-LTR retrotransposon protein and further comprises a donor template encoding a donor polynucleotide sequence for insertion into the target polynucleotide and located between two binding elements capable of forming a complex with the non-LTR retrotransposon protein.

In an example embodiment, comprises an integrase protein, and optionally a reverse transcriptase, and further comprises a donor template encoding a donor polynucleotide sequence for insertion into the target polynucleotide and located between two binding elements capable of forming a complex with the integrase protein. In an example embodiment, the composition further comprises a homologous recombination donor template comprising a donor polynucleotide sequence for insertion into a target polynucleotide.

In one aspect, as described herein, a vector system comprising one or more vectors encoding the hybrid CRISPR-Cas polypeptide and the guide RNA molecule. In one aspect, as described herein, one or more polynucleotides encoding one or more components of a composition described herein. In one aspect, as described herein, one or more vectors encoding the one or more polynucleotides described herein. In one aspect, as described herein, an engineered cell comprising a composition described herein. In one aspect, as described herein, a cell or progeny thereof genetically engineered to express one or more components of a compositions described herein.

In one aspect, as described herein, a method of modifying a target polynucleotide sequence in a cell, comprising introducing to the cell a composition described herein. In an example embodiment, the hybrid CRISPR-Cas polypeptide cleaves at a PAM comprising Ax, Tx, Gx, or xC wherein x of any nucleotide. In an example embodiment, the polypeptide and/or nucleic acid components are provided via one or more polynucleotides encoding the polypeptides and/or nucleic acid component(s), and wherein the one or more polynucleotides are operably configured to express the CRISPR-Cas hybrid polypeptide and/or the guide RNA molecule. In an example embodiment, the modifying comprises cleaving a DNA polynucleotide. In an example embodiment, contacting results in modification of a gene product or modification of the amount or expression of a gene product.

A method of editing nucleic acids in target polynucleotides comprising delivering a composition described herein, the one or more polynucleotides described herein to a cell or population of cells comprising the target polynucleotides. In an example embodiment, the target polynucleotides are target sequences within genomic DNA. In an example embodiment, the target polynucleotide is edited at one or more bases to introduce a G→A or C→T mutation.

In one aspect, as described herein, an isolated cell or progeny thereof comprising one or more base edits made using a method described herein.

In one aspect, as described herein, a method of modifying target polynucleotides comprising; delivering a composition described herein, the one or more polynucleotides described herein, or the one or more vectors described herein to a cell, or population of cells, comprising the target polynucleotide, wherein the complex directs the reverse transcriptase to the target sequence and the reverse transcriptase facilitates insertion of a donor sequence encoded by the donor template from the guide RNA molecule into the target polynucleotide.

In one aspect, as described herein, a method of modifying target polynucleotides comprising: delivering the composition described herein, the one or more polynucleotides described herein, or the one or more vectors described herein to a cell, or population of cells, comprising the target polynucleotide, wherein the complex directs the non-LTR retrotransposon protein to the target sequence and the non-LTR retrotransposon protein facilitates insertion of the donor polynucleotide sequence from the donor construct into the target polynucleotide.

In one aspect, as described herein, a method of modifying target polynucleotides comprising: delivering the composition described herein, the one or more polynucleotides described herein, or the one or more vectors described herein to a cell, or population of cells, comprising the target polynucleotide, wherein the complex directs the integrase protein to the target sequence and the integrase protein facilitates insertion of the donor polynucleotide sequence from the donor construct into the target polynucleotide.

In an example embodiment, insertion of the donor sequence introduces one or more base edits; corrects or introduces a premature stop codon; disrupts a splice site; inserts or restores a splice site; inserts a gene or gene fragment at one or both alleles of the target polynucleotide; or a combination thereof.

In one aspect, as describe herein, an isolated cell or progeny thereof comprising the modifications made using a method described herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1—Example discovery pipeline identifying Cas5-HNH.

FIG. 2—Cas5-HINH are active in an E. coli PAM interference assay.

FIG. 3—Cas5-HNH System has PAM and Processed CRISPR RNA.

FIG. 4—Cas5-HNH Cascade RNP precisely cleaves dsDNA in vitro in a target- and PAM-dependent manner.

FIG. 5—Cas5-HNH HEK-293 Genome Editing. Cas5-HNH Cascade can be used for genome editing in human cells.

FIG. 6—Cas5-HNH HEK-293 Genome Editing, HNH Mutant and Component Dropout. HNH catalytic residues are necessary for DNA cleavage activity in human cells.

FIG. 7A-7B—Cas5-HNH is a novel Type I CRISPR system. Loci lack Cas3, but the Cas5 has an active HNH domain inserted at the C-terminus. 7A) Example locus from contig MWEE01000013. 7B) (SEQ ID NO: 66-71) Alignment of proteins from 6 representative proteins.

FIG. 8—RNP purification of Cas5-HNH Cascade. Lanes) 1: Total lysate, 2: Soluble fraction, 3: Insoluble fraction, 4: Column flow through, 5: Elution, L: BioRad precision plus protein dual ladder. Expected sizes of Cascade proteins) Cas8: 60.57 kDa, Cas11 (His-tagged): 24.68 kDa, Cas7: 41.74 kDa, Cas5-HNH: 43.55 kDa, Cas6: 31.17 kDa.

FIG. 9A-9M—HNH-functionalized Cascade subunits perform precise, RNA-guided dsDNA cleavage. (9A) Locus diagram of the experimentally studied Cas8-HNH system from Selenomonas sp. isolate RGIG9219. (9B) Locus diagram of the experimentally studied Cas5-HNH system from Candidatus Cloacimonetes bacterium. (9C) Sequence logo for the PAM of Cas8-HNH as determined by a plasmid depletion assay in E. coli. (9D) Sequence logo for the PAM of Cas5-HNH as determined by a plasmid depletion assay in E. coli. (9E) (SEQ ID NO: 72) Small RNA-seq of Cas8-HNH Cascade RNP pulldown. (9F) (SEQ ID NO: 73) Small RNA-seq of Cas5-HNH Cascade RNP pulldown. (9G) In vitro reconstituted Cas8-HNH Cascade RNP cleavage of linear dsDNA targets, in the presence or absence of a cognate target and/or PAM. (9H) In vitro reconstituted Cas5-HNH Cascade RNP cleavage of linear dsDNA targets, in the presence or absence of a cognate target and/or PAM. (9I) (SEQ ID NO: 74-75) Sanger sequencing of cleavage products generated by Cas8-HNH. (9J) (SEQ ID NO: 76-77) Sanger sequencing of cleavage products generated by Cas5-HNH. In both (9I) and (9J), the polymerase used exhibits non-templated incorporation of a terminal adenine, which results in a thymidine appearing at the end of the trace. (9K) HEK293FT genome editing at 4 genomic loci by Cas8-HNH in the presence or absence of each Cascade subunit or cognate guideRNA, or with alanine mutation of HNH domain catalytic residues. Error bars denote SD. * P<0.05 relative to non-targeting (NT) guide condition. (9L) HEK293FT genome editing at 4 genomic loci by Cas5-HINH in the presence or absence of each Cascade subunit or cognate guideRNA, or with alanine mutation of HNH domain catalytic residues. Error bars denote SD. * P<0.05 relative to non-targeting (NT) guide condition. (9M) TTISS off-target analysis of Cas8-HNH genome editing in HEK293FT cells for 4 guides.

FIG. 10A-10B—(10A) CRISPR-Cas effector modules identified in this study. All enhanced CRISPR association scores are shown below the system name as determined by the pipeline with the numerator indicating the number of CRISPR/divergent DR associated loci and the denominator indicating the effective sample size of the cluster. HNH: Nuclease domain with HNH or HNN catalytic. (10B) General evolutionary mechanisms that likely gave rise to the diverse CRISPR-Cas effector modules identified previously and in this study.

FIG. 11A-11B—Affinity purification of Cas8-HNH and Cas5-HNH effector complexes SDS-PAGE gel electrophoresis of affinity purified (11A) Cas8-HNH and (11B) Cas5-HNH effector RNP complexes.

FIG. 12—Alphafold structures showing Cas11 darker/right protein)—Cas5-HNH (grey/left protein) interaction. HNH is toward the bottom of the picture.

FIG. 13—Alphafold structures showing Cas6 (bottom protein)—Cas5-HNH (top and middle protein) interaction. HNH is the globular domain located on the bottom of the grey protein.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2^{nd} ed., J. Wiley & Sons (New York, N.Y. 1994), March, Advanced Organic Chemistry Reactions, Mechanisms and Structure 4^{th} ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### OVERVIEW

In one aspect, the present disclosure provides for systems and compositions for modification of nucleic acids. In general, the systems or compositions comprises at least a Cas5 or Cas8 polypeptide comprising one or more HINH domains, optionally in combination with one or more other polypeptides, collectively the Cas5-HNH or Cas8-HNH polypeptide, and a guide molecule capable of forming a complex with the Cas5-HNH or Cas8-HNH polypeptide and directing site-specific binding of the Cas5-HNH or Cas8-HNH complex to a target polynucleotide. In another aspect the present disclosure is directed to vectors and other delivery composition used to deliver the Cas5-HNH or Cas8-HNH and/or guide molecule to a cell or population of cells in vitro, ex vivo, or in vivo. In another aspect, the present disclosure is directed to method of modifying target polynucleotides using the Cas5-HNH or Cas8-HNH compositions disclosed herein, including the use of catalytically inactive or nickase variants of Cas5-HNH or Cas8-HNH coupled with other functional domains to further expand the gene editing capabilities of the Cas5-HNH or Cas8-HNH compositions and systems disclosed herein.

### Cas5-HNH Complexes

The Cas5-HNH systems are a complex of one or more Cas polypeptides and a guide molecule. The complex comprises at least one Cas5-HINH polypeptide. The systems may further comprise a Cas8, a Cas11, a Cas7, a Cas6, or a Cas1 polypeptide or any combination thereof. In one example embodiment, the Cas5-HNH complex does not comprise a Cas7, Cas11, Cas12, or any combination thereof. The Cas-HNH polypeptide (used interchangeably herein with “Cas”, “Cas effector”, “Cas protein”) may include Cas-HNH polypeptides that have at least one HNH domain. In example embodiments, the Cas-HNH polypeptide may comprise only one HNH domain.

### Cas5-HINH Polypeptides

Cas5 was previously known to be a component of certain Type 1 systems, where it formed part of the RAMP (Repeat-Associated Mysterious Proteins) involved in binding a guide molecule. See Koonin et al. “Diversity, classification and evolution of CRISPR-Cas systems” Curr Opin Microbiol. 2017, 37:67-78 and Makarova et al., Annotation and Classification of CRISPR-Cas Systems, Methods Mol Biol. 2015; 1311:47-75. The RAMP backbone in Type 1 systems typically comprise one Cas5 subunit and several Cas7 subunits with the Cas5 subunit binding a 5′ handle of the crRNA and interacting with the large subunit e.g., Cas8. Id. In contrast to known Cas5 polypeptides and Type 1 systems, the Cas5-HNH polypeptides disclosed herein further comprise a HNH endonuclease domain capable of double stranded and/or single stranded DNA cleavage. In example embodiments, the HNH domain is located at the C-terminus. Cas5 has not previously been recognized as capable of possessing an endonuclease domain or possessing single-strand or double-strand DNA cleavage capability.

Nucleic acid sequences of example Cas5-HINH polypeptides include those in and SEQ ID NOs. 1-10, 81-83, and 89-94. In particular, Cas5-HNH can be found at position: 25,000-26,136 of SEQ ID NO 1; 25,000-26,097 of SEQ ID NO 2; 25,00-26,109 of SEQ ID NO 3; 25,00-26,160 of SEQ ID NO 4; 25,00-26,142 of SEQ ID NO 5; 25,00-26,094 of SEQ ID NO 6; 25,00-26,097 of SEQ ID NO 7; 25,00-26,106 of SEQ ID NO 8; 25,00-26,118 of SEQ ID NO 9; 25,00-26,116 of SEQ ID NO 10; 4838-6004 of SEQ ID NO 81; 8323-9489 of SEQ ID NO 82; 1974-3581 of SEQ ID NO 89; 1974-2498 of SEQ ID NO 90; 1974-3110 of SEQ ID NO 91; 1974-3140 of SEQ ID NO 92; 1974-2792 of SEQ ID NO 93.

As used herein, when a Cas5-HNH polypeptide originates from a species, it may be the wild-type Cas protein in the species, or a homolog of the wild-type Cas protein in the species. The Cas polypeptide that is a homolog of the wild-type Cas protein in the species may comprise one or more variations (e.g., mutations, truncations, etc.) of the wild-type Cas protein.

The systems and compositions may comprise orthologs and homologs of the Cas5-HINH proteins. The terms “ortholog” and “homolog” are well known in the art. By means of further guidance, a “homolog” refers to two genes that share a common ancestral gene. Homologous proteins may but need not be structurally related or are only partially structurally related. An “ortholog” are two genes that share common ancestral gene but occur in different species. Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April;22 (4): 359-66. doi: 10.1002/pro.2225.). See also Shmakov et al. (2015) for application in the field of CRISPR-Cas loci. Homologous proteins may but need not be structurally related or are only partially structurally related.

In one embodiment, the homolog or ortholog of a Cas5-HINH polypeptide such as referred to herein has a sequence homology or identity of at least 60%, of at least 70%, of at least 80%, at least 85%, at least 90%, at least 95% with a Cas5-HNH protein. In further embodiments, the homolog or ortholog of a Cas5-HINH polypeptide has a sequence identity of at least 80%, at least 85%, at least 90%, or at least 95% with a wildtype Cas5-HNH protein, in particular embodiment the Cas5-HNH sequence identified in SEQ ID NOs 1-8.

In example embodiments, the Cas5 polypeptide is less than 2000 amino acids in size. For example, the Cas polypeptide may be less than 2000, less than 1900, less than 1800, less than 1700, less than 1600, less than 1500, less than 1400, less than 1300, less than 1200, less than 1100, less than 1000, less than 950, less than 900, less than 890, less than 880, less than 870, less than 860, less than 850, less than 840, less than 830, less than 820, less than 810, less than 800, less than 790, less than 780, less than 770, less than 760, less than 750, less than 700, less than 650, less than 600 amino acids in size. In some examples, the Cas polypeptide is less than 600 amino acids in size.

In example embodiments, the hybrid CRISPR-Cas polypeptide comprises the amino acids corresponding to the about 1,000 to about 1,200 nucleic acids encoding the protein. In example embodiments, the hybrid CRISPR-Cas polypeptide comprises the amino acids corresponding to the about 1,000 to about 1,100 nucleic acids encoding the protein. In example embodiments, the hybrid CRISPR-Cas polypeptide comprises the amino acids corresponding to the about 1,100 to about 1,200 nucleic acids encoding the protein. In example embodiments, the hybrid CRISPR-Cas polypeptide comprises the amino acids corresponding to less than 2000 amino acids. In example embodiments, the hybrid CRISPR-Cas polypeptide comprises the amino acids corresponding to less than 1500 amino acids. In example embodiments, the hybrid CRISPR-Cas polypeptide comprises the amino acids corresponding to less than 1200 amino acids. In example embodiments, the hybrid CRISPR-Cas polypeptide comprises the amino acids corresponding to more than 900 amino acids.

### Cas8-HNH Complexes

The Cas8-HNH systems are a complex of one or more Cas polypeptides and a guide molecule. The complex comprises at least one Cas8-HNH polypeptide. The systems may further comprise a Cas5, a Cas7, or a Cas6 polypeptide or any combination thereof. The Cas-HNH polypeptide (used interchangeably herein with “Cas”, “Cas effector”, “Cas protein”) may include Cas-HNH polypeptides that have at least one HNH domain. In example embodiments, the Cas-HNH polypeptide may comprise only one HNH domain.

### Cas8-HINH Polypeptides

Cas8 was previously known to be a component of certain Type 1 systems, where it is is a candidate PAM recognition factor and plays a role in interference. See Cass, S. D. B.; Haas, K. A.; Stoll, B.; Alkhnbashi, O. S.; Sharma, K.; Urlaub, H.; Backofen, R.; Marchfelder, A.; Bolt, E. L. The Role of Cas8 in Type I CRISPR Interference. Bioscience Reports, 2015, 35. Cas8 interacts with a Cas5-Cas7-crRNA complex and stimulates binding to substrates comprising the corresponding PAM. In some instances, the Cas8 protein comprises of Cas8′, Cas8″, or both. In Type I systems, a Cas5 is embedded in a Cas8 to form the base complex further comprising a backbone of multiple Cas7 subunits. In contrast to known Cas8 polypeptides and Type 1 systems, the Cas8-HNH polypeptides disclosed herein further comprise a HNH endonuclease domain capable of double stranded and/or single stranded DNA cleavage. In example embodiments, the HNH domain is located at the C-terminus. Cas8 has not previously been recognized as capable of possessing an endonuclease domain or possessing single-strand or double-strand DNA cleavage capability.

In example embodiments, the Cas8 is a Cas8e. Cas8e was previously known to be a component of certain Type I-E systems residing in the N-terminal domain. Cas8e comprises of three distinct structural features: a glutamine wedge, a glycine loop, and a lysine finger, which recognizes a PAM sequence. See e.g., Gleditzsch, D.; Pausch, P.; Müller-Esparza, H.; Özcan, A.; Guo, X.; Bange, G.; Randau, L. PAM Identification by CRISPR-Cas Effector Complexes: Diversified Mechanisms and Structures. RNA Biology, 2018, 16, 504-517.

Nucleic acid sequences of example Cas8-HINH polypeptides include those in and SEQ ID NOs. 78-80, G-K. In particular, Cas8-HNH can be found at position 1973:3007 of SEQ ID NO 78; 6522:7676 of SEQ ID NO 79; 1974:2981 of SEQ ID NO 84; 1974:2519 of SEQ ID NO 85; 1974:3008 of SEQ ID NO 86; 1974:2738 of SEQ ID NO 87.

As used herein, when a Cas8-HNH polypeptide originates from a species, it may be the wild-type Cas protein in the species, or a homolog of the wild-type Cas protein in the species. The Cas polypeptide that is a homolog of the wild-type Cas protein in the species may comprise one or more variations (e.g., mutations, truncations, etc.) of the wild-type Cas protein.

The systems and compositions may comprise orthologs and homologs of the Cas8-HNH proteins. The terms “ortholog” and “homolog” are well known in the art. By means of further guidance, a “homolog” refers to two genes that share a common ancestral gene. Homologous proteins may but need not be structurally related or are only partially structurally related. An “ortholog” are two genes that share common ancestral gene but occur in different species. Orthologous proteins may but need not be structurally related or are only partially structurally related. Homologs and orthologs may be identified by homology modelling (see, e.g., Greer, Science vol. 228 (1985) 1055, and Blundell et al. Eur J Biochem vol 172 (1988), 513) or “structural BLAST” (Dey F, Cliff Zhang Q, Petrey D, Honig B. Toward a “structural BLAST”: using structural relationships to infer function. Protein Sci. 2013 April;22 (4): 359-66. doi: 10.1002/pro.2225.). See also Shmakov et al. (2015) for application in the field of CRISPR-Cas loci. Homologous proteins may but need not be structurally related or are only partially structurally related.

In one embodiment, the homolog or ortholog of a Cas8-HINH polypeptide such as referred to herein has a sequence homology or identity of at least 60%, of at least 70%, of at least 80%, at least 85%, at least 90%, at least 95% with a Cas8-HNH protein. In further embodiments, the homolog or ortholog of a Cas8-HNH polypeptide has a sequence identity of at least 80%, at least 85%, at least 90%, or at least 95% with a wildtype Cas8-HNH protein, in a particular embodiment the Cas8-HNH sequence identified in SEQ ID NO: 78.

In example embodiments, the Cas8 polypeptide is less than 2000 amino acids in size. For example, the Cas polypeptide may be less than 2000, less than 1900, less than 1800, less than 1700, less than 1600, less than 1500, less than 1400, less than 1300, less than 1200, less than 1100, less than 1000, less than 950, less than 900, less than 890, less than 880, less than 870, less than 860, less than 850, less than 840, less than 830, less than 820, less than 810, less than 800, less than 790, less than 780, less than 770, less than 760, less than 750, less than 700, less than 650, less than 600 amino acids in size, less than 550 amino acids in size, less than 500 amino acids in size, less than 450 amino acids in size, less than 400 amino acids in size. In some examples, the Cas polypeptide is less than 400 amino acids in size.

In example embodiments, the hybrid CRISPR-Cas polypeptide comprises the amino acids corresponding to the about 1,000 to about 1,200 nucleic acids encoding the protein. In example embodiments, the hybrid CRISPR-Cas polypeptide comprises the amino acids corresponding to the about 1,000 to about 1,100 nucleic acids encoding the protein. In example embodiments, the hybrid CRISPR-Cas polypeptide comprises the amino acids corresponding to the about 1,100 to about 1,200 nucleic acids encoding the protein. In example embodiments, the hybrid CRISPR-Cas polypeptide comprises the amino acids corresponding to less than 2000 amino acids. In example embodiments, the hybrid CRISPR-Cas polypeptide comprises the amino acids corresponding to less than 1500 amino acids. In example embodiments, the hybrid CRISPR-Cas polypeptide comprises the amino acids corresponding to less than 1200 amino acids. In example embodiments, the hybrid CRISPR-Cas polypeptide comprises the amino acids corresponding to more than 900 amino acids.

### HNH Domain

The HNH domain comprises two antiparallel beta strands connected with a variable length loop, an alpha helix, with a metal binding site between the two. The size of the domain varies, but by way of an example, the Cas9 HNH domain is approximately 130 amino acids in length. The HNH conserved sites are conserved across the HNH superfamily, with HNH conservation throughout bacteria. In Cas9 proteins, for example, the HNH domain comprises a two-stranded antiparallel β-sheet (β12 and β13) flanked by four α-helices (α35-α38). It shares structural similarity with the HNH endonucleases characterized by a ββα-metal fold, such as phage T4 endonuclease VII (Endo VII) (PDB code 2QNC, 20% identity, rmsd of 2.7 Å for 61 equivalent Cα atoms) and Vibrio vulnificus nuclease (PDB code 1OUP, 8% identity, rmsd of 2.7 Å for 77 equivalent Cαatoms). HNH nucleases have three catalytic residues (e.g., Asp40, His41, and Asn62 in Endo VII), and cleave nucleic acid substrates through a single-metal mechanism. In the structure of the Endo VII N62D mutant in complex with a Holliday junction, a Mg2+ ion is coordinated by Asp40, Asp62, and the oxygen atoms of the scissile phosphate group of the substrate, while His41 acts as a general base to activate a water molecule for catalysis. Asp839, His840, and Asn863 of the Cas9 HNH domain correspond to Asp40, His41, and Asn62 of Endo VII, respectively, consistent with the observation that His840 is critical for the cleavage of the complementary DNA strand. The N863A mutant functions as a nickase, indicating that Asn863 participates in catalysis. The Cas9 HNH domain may cleave the complementary strand of the target DNA through a single-metal mechanism, as observed for other HINH superfamily nucleases. Although the Cas9 HNH domain shares a ββα-metal fold with other HNH endonucleases, their overall structures are distinct, consistent with the differences in their substrate specificities. Accordingly, Cas5 or Cas8 polypeptides of the present invention may comprise similar HNH domains in terms of sequence and/or function and may likewise comprise mutations analogous to those described above for Cas9 which convert the Cas5 or Cas8 polypeptide to a nickase. See e.g., Nishimasu H., et al. Crystal structure of Cas9 in complex with guide RNA and target DNA. Cell 156 (5), 935-949.

### Guide Sequences

The Cas5-HNH or Cas8-HNH polypeptide herein may further comprise one or more guide sequences. A guide sequence may form a complex with a nucleic acid-guided nuclease and direct the complex to bind with a target sequence. In some examples, the guide sequence may comprise a first and second nucleic acid molecules, the first and second nucleic acid molecules capable of forming a duplex, the duplex capable of forming a complex with the nucleic acid-guided nuclease, wherein the second nucleic acid molecule is a recombinant molecule comprising a heterologous guide sequence capable of directing site-specific binding of the complex to a target sequence of a target polynucleotide. In some examples, the single guide sequence capable of forming a complex with the nucleic acid-guided nuclease and directing site-specific binding of the complex to a target sequence of a target polynucleotide.

As used herein, a heterologous guide sequence is a guide sequence that is not derived from the same species as the nucleic acid-guided nuclease. For example, a heterologous guide sequence of a nucleic acid-guided nuclease derived from species A is a polynucleotide derived from a species different from species A, or an artificial polynucleotide.

As used herein, the term “guide sequence”, “guide sequence RNA”, or “guide RNA molecule” has the meaning as used herein elsewhere and comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In one embodiment, the degree of complementarity of the guide sequence to a given target sequence, when optimally aligned using a suitable alignment algorithm, is about or more than 50%, 60%, 75%, 80%, 85%, 90%, 95%, 97.5%, 99%, or more. In certain example embodiments, the guide sequence comprises a guide sequence that may be designed to have at least one mismatch with the target sequence, such that an RNA duplex formed between the guide sequence and the target sequence. Accordingly, the degree of complementarity is less than 99%. For instance, where guide sequence consists of 24 nucleotides, the degree of complementarity is more particularly about 96% or less. In one embodiment, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In one embodiment, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-guided nuclease-guide system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test guide sequence reactions. Other assays are possible, and will occur to those skilled in the art. A guide sequence, and hence a nucleic acid-targeting guide RNA may be selected to target any target nucleic acid sequence.

A guide sequence, and hence a nucleic acid-targeting guide, may be selected to target any target nucleic acid sequence. The target sequence may be DNA. The target sequence may be any RNA sequence. In one embodiment, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In an embodiment, the guide sequence or spacer length of the guide sequence is from 15 to 50 nt. In an embodiment, the spacer length of the guide sequence RNA at least 15 nucleotides. In an embodiment, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer. In certain example embodiment, the guide sequence is 15, 16, 17,18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In one embodiment, the sequence of the guide sequence (direct repeat and/or spacer) is selected to reduce the degree secondary structure within the guide molecule. In one embodiment, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example of a folding algorithm is the online webserver RNAfold, developed at Institute for Theoretical Chemistry at the University of Vienna, using the centroid structure prediction algorithm (see e.g., A. R. Gruber et al., 2008, Cell 106 (1): 23-24; and PA Carr and GM Church, 2009, Nature Biotechnology 27 (12): 1151-62). Further algorithms may be found in U.S. application Ser. No. TBA (attorney docket 44790.11.2022; Broad Reference BI-2013/004A); incorporated herein by reference.

In a particular embodiment, the guide sequence comprises a guide sequence linked to a direct repeat sequence, wherein the direct repeat sequence comprises one or more stem loops or optimized secondary structures. In one embodiment, the direct repeat has a minimum length of 16 nts and a single stem loop. In further embodiments the direct repeat has a length longer than 16 nts, preferably more than 17 nts, and has more than one stem loops or optimized secondary structures. In one embodiment, the guide sequence comprises or consists of the guide sequence linked to all or part of the natural direct repeat sequence. In one embodiment, certain aspects of the guide sequence architecture can be modified, for example by addition, subtraction, or substitution of features, whereas certain other aspects of guide sequence architecture are maintained. Preferred locations for engineered guide sequence modifications, including but not limited to insertions, deletions, and substitutions include guide sequence termini and regions of the guide sequence that are exposed when complexed with nucleic acid-guided nuclease and/or target, for example the tetraloop and/or loop2.

In one embodiment, a loop in the guide sequence RNA is provided. This may be a stem loop or a tetra loop. The loop is preferably GAAA, but it is not limited to this sequence or indeed to being only 4 bp in length. Indeed, preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG.

In one embodiment, the guide sequence forms a stem loop with a separate non-covalently linked sequence, which can be DNA or RNA. In one embodiment, the sequences forming the guide sequence are first synthesized using the standard phosphoramidite synthetic protocol (Herdewijn, P., ed., Methods in Molecular Biology Col 288, Oligonucleotide Synthesis: Methods and Applications, Humana Press, New Jersey (2012)). In one embodiment, these sequences can be functionalized to contain an appropriate functional group for ligation using the standard protocol known in the art (Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In one embodiment, these stem-loop forming sequences can be chemically synthesized. In one embodiment, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

The repeat: anti repeat duplex will be apparent from the secondary structure of the sgRNA. It may be typically a first complimentary stretch after (in 5′ to 3′ direction) the poly U tract and before the tetraloop; and a second complimentary stretch after (in 5′ to 3′ direction) the tetraloop and before the poly A tract. The first complimentary stretch (the “repeat”) is complimentary to the second complimentary stretch (the “anti-repeat”). As such, they Watson-Crick base pair to form a duplex of dsRNA when folded back on one another. As such, the anti-repeat sequence is the complimentary sequence of the repeat and in terms to A-U or C-G base pairing, but also in terms of the fact that the anti-repeat is in the reverse orientation due to the tetraloop.

In an embodiment of the invention, modification of guide sequence architecture comprises replacing bases in stem loop 2. For example, in one embodiment, “actt” (“acuu” in RNA) and “aagt” (“aagu” in RNA) bases in stemloop2 are replaced with “cgcc” and “gcgg”. In one embodiment, “actt” and “aagt” bases in stemloop2 are replaced with complimentary GC-rich regions of 4 nucleotides. In one embodiment, the complimentary GC-rich regions of 4 nucleotides are “cgcc” and “gcgg” (both in 5′ to 3′ direction). In one embodiment, the complimentary GC-rich regions of 4 nucleotides are “gcgg” and “cgcc” (both in 5′ to 3′ direction). Other combination of C and G in the complimentary GC-rich regions of 4 nucleotides will be apparent including CCCC and GGGG.

In one aspect, the stem loop 2, e.g., “ACTTgtttAAGT” (SEQ ID NO: 11) can be replaced by any “XXXXgtttYYYY”, e.g., where XXXX and YYYY represent any complementary sets of nucleotides that together will base pair to each other to create a stem.

In one aspect, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-12 and Y2-12 (wherein X and Y represent any complementary set of nucleotides) may be contemplated. In one aspect, the stem made of the X and Y nucleotides, together with the “gttt,” will form a complete hairpin in the overall secondary structure; and this may be advantageous and the amount of base pairs can be any amount that forms a complete hairpin. In one aspect, any complementary X:Y base pairing sequence (e.g., as to length) is tolerated, so long as the secondary structure of the entire guide sequence sgRNA is preserved. In one aspect, the stem can be a form of X:Y base pairing that does not disrupt the secondary structure of the whole guide sequence sgRNA in that it has a DR: tracr duplex, and 3 stem loops. In one aspect, the “gttt” tetraloop that connects ACTT and AAGT (or any alternative stem made of X:Y base pairs) can be any sequence of the same length (e.g., 4 base pair) or longer that does not interrupt the overall secondary structure of the sgRNA. In one aspect, the stem loop can be something that further lengthens stemloop2, e.g., can be MS2 aptamer. In one aspect, the stemloop3 “GGCACCGagtCGGTGC” (SEQ ID NO: 12) can likewise take on a XXXXXXXagtYYYYYYY” form, e.g., wherein X7 and Y7 represent any complementary sets of nucleotides that together will base pair to each other to create a stem. In one aspect, the stem comprises about 7 bp comprising complementary X and Y sequences, although stems of more or fewer base pairs are also contemplated. In one aspect, the stem made of the X and Y nucleotides, together with the “agt”, will form a complete hairpin in the overall secondary structure. In one aspect, any complementary X:Y base pairing sequence is tolerated, so long as the secondary structure of the entire sgRNA is preserved. In one aspect, the stem can be a form of X:Y base pairing that doesn't disrupt the secondary structure of the whole sgRNA in that it has a DR: tracr duplex, and 3 stem loops. In one aspect, the “agt” sequence of the stem loop 3 can be extended or be replaced by an aptamer, e.g., a MS2 aptamer or sequence that otherwise generally preserves the architecture of stemloop3. In one aspect for alternative Stem loops 2 and/or 3, each X and Y pair can refer to any base pair. In one aspect, non-Watson Crick base pairing is contemplated, where such pairing otherwise generally preserves the architecture of the stem loop at that position.

In one aspect, the DR:tracrRNA duplex can be replaced with the form: gYYYYag (N) NNNNxxxxNNNN (AAN) uuRRRRu (SEQ ID NO: 13) (using standard IUPAC nomenclature for nucleotides), wherein (N) and (AAN) represent part of the bulge in the duplex, and “xxxx” represents a linker sequence. NNNN on the direct repeat can be anything so long as it base pairs with the corresponding NNNN portion of the tracrRNA. In one aspect, the DR:tracrRNA duplex can be connected by a linker of any length, any base composition, as long as it doesn't alter the overall structure.

In one embodiment, the natural hairpin or stem loop structure of the guide sequence is extended or replaced by an extended stem loop. Extension of the stem can enhance the assembly of the guide sequence with the nucleic acid-guided nuclease. In one embodiment the stem of the stem loop is extended by at least 1, 2, 3, 4, 5 or more complementary base pairs (i.e., corresponding to the addition of 2,4, 6, 8, 10 or more nucleotides in the guide sequence). In one embodiment these are located at the end of the stem, adjacent to the loop of the stem loop.

In one embodiment, the susceptibility of the guide sequence to RNAses or to decreased expression can be reduced by slight modifications of the sequence of the guide sequence which do not affect its function. For instance, in one embodiment, premature termination of transcription, such as premature transcription of U6 Pol-III, can be removed by modifying a putative Pol-III terminator (4 consecutive U's) in the guide sequence. Where such sequence modification is required in the stem loop of the guide sequence, it is preferably ensured by a base pair flip.

In an embodiment, the guide sequence comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide sequence nucleic acid comprises ribonucleotides and non-ribonucleotides. In one such embodiment, a guide sequence comprises one or more ribonucleotides and one or more deoxyribonucleotides. In an embodiment of the invention, the guide sequence comprises one or more non-naturally occurring nucleotide or nucleotide analog such as a nucleotide with phosphorothioate linkage, a locked nucleic acid (LNA) nucleotide comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified guide sequence can comprise increased stability and increased activity as compared to unmodified guide sequence, though on-target vs. off-target specificity is not predictable. (See, Hendel, 2015, Nat Biotechnol. 33 (9): 985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33 (9): 985-989; Li et al., Nature Biomedical Engineering, 2017, 1, 0066 DOI: 10.1038/s41551-017-0066). In one embodiment, the 5′ and/or 3′ end of a guide sequence RNA is modified by a variety of functional moieties including fluorescent dyes, polyethylene glycol, cholesterol, proteins, or detection tags. (See Kelly et al., 2016, J. Biotech. 233:74-83). In an embodiment, a guide sequence comprises ribonucleotides in a region that binds to a target sequence and one or more deoxyribonucletides and/or nucleotide analogs in a region that binds to the nucleic acid-guided nuclease. In an embodiment, deoxyribonucleotides and/or nucleotide analogs are incorporated in engineered guide structures, such as, without limitation, stem-loop regions, and the seed region. In one embodiment, 3-5 nucleotides at either the 3′ or the 5′ end of a guide is chemically modified. In one embodiment, only minor modifications are introduced in the seed region, such as 2′-F modifications. In one embodiment, 2′-F modification is introduced at the 3′ end of a guide. In an embodiment, three to five nucleotides at the 5′ and/or the 3′ end of the guide sequence are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see Hendel et al., Nat. Biotechnol. (2015) 33 (9): 985-989). In an embodiment, all the phosphodiester bonds of a guide sequence are substituted with phosphorothioates (PS) for enhancing levels of gene disruption. In an embodiment, more than five nucleotides at the 5′ and/or the 3′ end of the guide sequence are chemically modified with 2′-O-Me, 2′-F or S-constrained ethyl (cEt). Such chemically modified guide sequence can mediate enhanced levels of gene disruption (see Ragdarm et al., 0215, PNAS, E7110-E7111). In an embodiment of the invention, a guide sequence is modified to comprise a chemical moiety at its 3′ and/or 5′ end. Such moieties include, but are not limited to amine, azide, alkyne, thio, dibenzocyclooctyne (DBCO), or Rhodamine. In certain embodiment, the chemical moiety is conjugated to the guide sequence by a linker, such as an alkyl chain. In an embodiment, the chemical moiety of the modified guide sequence can be used to attach the guide sequence to another molecule, such as DNA, RNA, protein, or nanoparticles. Such chemically modified guide sequence can be used to identify or enrich cells generically edited by a nucleic acid-guided nuclease and related systems (see Lee et al., eLife, 2017, 6:e25312, DOI: 10.7554).

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In one embodiment, the nucleic acid-guided nuclease may need a tracr sequence. The “tracrRNA” sequence or analogous terms includes any polynucleotide sequence that has sufficient complementarity with a crRNA sequence to hybridize. In one embodiment, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In one embodiment, the tracr sequence is about or more than about 5, 6, 7, 8, 9, 10, 11, 12, 13, 14, 15, 16, 17, 18, 19, 20, 25, 30, 40, 50, or more nucleotides in length. In one embodiment, the tracr sequence and guide sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop may correspond to the tracr mate sequence, and the portion of the sequence 3′ of the loop then corresponds to the tracr sequence. In a hairpin structure the portion of the sequence 5′ of the final “N” and upstream of the loop may alternatively correspond to the tracr sequence, and the portion of the sequence 3′ of the loop corresponds to the tracr mate sequence.

In one embodiment, the tracr and tracr mate sequences can be chemically synthesized. In one embodiment, the chemical synthesis uses automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

In one embodiment, the tracr and tracr mate sequences can be covalently linked using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8:570-9; Behlke et al., Oligonucleotides (2008) 18:305-19; Watts, et al., Drug. Discov. Today (2008) 13:842-55; Shukla, et al., ChemMedChem (2010) 5:328-49.

In one embodiment, the tracr and tracr mate sequences can be covalently linked using click chemistry. In one embodiment, the tracr and tracr mate sequences can be covalently linked using a triazole linker. In one embodiment, the tracr and tracr mate sequences can be covalently linked using Huisgen 1,3-dipolar cycloaddition reaction involving an alkyne and azide to yield a highly stable triazole linker (He et al., ChemBioChem (2015) 17:1809-1812; WO 2016/186745). In one embodiment, the tracr and tracr mate sequences are covalently linked by ligating a 5′-hexyne tracrRNA and a 3′-azide crRNA. In one embodiment, either or both of the 5′-hexyne tracrRNA and a 3′-azide crRNA can be protected with 2′-acetoxyethl orthoester (2′-ACE) group, which can be subsequently removed using Dharmacon protocol (Scaringe et al., J. Am. Chem. Soc. (1998) 120:11820-11821; Scaringe, Methods Enzymol. (2000) 317:3-18).

In one embodiment, the tracr and tracr mate sequences can be covalently linked via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electrochemiluminescent, and bioluminescent marker compounds. The design of example linkers conjugating two RNA components are also described in WO 2004/015075.

The linker (e.g., a non-nucleotide loop) can be of any length. In one embodiment, the linker has a length equivalent to about 0-16 nucleotides. In one embodiment, the linker has a length equivalent to about 0-8 nucleotides. In one embodiment, the linker has a length equivalent to about 0-4 nucleotides. In one embodiment, the linker has a length equivalent to about 2 nucleotides. Example linker design is also described in International Patent Publication No. WO 2011/008730.

In an embodiment, the nucleic acid-guided nuclease uses of a tracrRNA, the guide sequence, tracr mate, and tracr sequence may reside in a single RNA, i.e., an sgRNA (arranged in a 5′ to 3′ orientation or alternatively arranged in a 3′ to 5′ orientation), or the tracr RNA may be a different RNA than the RNA containing the guide sequence and tracr mate sequence. In these embodiments, the tracr hybridizes to the tracr mate sequence and directs the nucleic acid-guided nuclease-guide molecule complex to the target sequence. In some examples, a guide sequence comprises (in 5′ to 3′ direction): a guide sequence, a poly U tract, a first complimentary stretch (the “repeat”), a loop (tetraloop), a second complimentary stretch (the “anti-repeat” being complimentary to the repeat), a stem, and further stem loops and stems and a poly A (often poly U in RNA) tail (terminator). In preferred embodiments, certain aspects of guide sequence architecture are retained, certain aspect of guide sequence architecture cam be modified, for example by addition, subtraction, or substitution of features, whereas certain other aspects of guide sequence architecture are maintained. Preferred locations for engineered guide sequence modifications, including but not limited to insertions, deletions, and substitutions include guide sequence termini and regions of the guide sequence that are exposed when complexed with nucleic acid-guided nuclease and/or target, for example the tetraloop and/or loop2.

In one embodiment, the guide sequence comprises, in addition the guide sequence, a sequence corresponding to a direct repeat in the CRISPR locus. In one embodiment, this sequence comprises at least one hairpin, i.e., a region of self-complementarity. In one embodiment, the guide sequence is 3′ of the direct repeat comprising at least one hairpin. In further embodiments, the guide sequence is 5′ of the direct repeat comprising at least one hairpin. In one embodiment, a hairpin is located in the middle of the guide sequence, i.e., the guide sequence is in part 5′ and in part 3′ of the direct repeat. The hairpin in the middle of the guide sequence may be involved in recognition or processing of the guide molecule. In one embodiment, the hairpin structure comprises at least 5, preferably 7-20 nucleotides.

### Guide Chemical Modifications

In an embodiment, the molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide sequence nucleic acid comprises ribonucleotides and non-ribonucleotides. In one such embodiment, a guide sequence comprises one or more ribonucleotides and one or more deoxyribonucleotides. In an embodiment of the invention, the guide sequence comprises one or more non-naturally occurring nucleotide or nucleotide analog such as a nucleotide with phosphorothioate linkage, a locked nucleic acid (LNA) nucleotide comprising a methylene bridge between the 2′ and 4′ carbons of the ribose ring, or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl (cEt), or 2′-O-methyl 3′thioPACE (MSP) at one or more terminal nucleotides. Such chemically modified guide RNA can comprise increased stability and increased activity as compared to unmodified guide sequence, though on-target vs. off-target specificity is not predictable. (See, Hendel, 2015, Nat Biotechnol. 33 (9): 985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33 (9): 985-989; Li et al., Nature Biomedical Engineering, 2017, 1, 0066 DOI: 10.1038/s41551-017-0066). In one embodiment, the 5′ and/or 3′ end of a guide sequence is modified by a variety of functional moieties including fluorescent dyes, polyethylene glycol, cholesterol, proteins, or detection tags. (See Kelly et al., 2016, J. Biotech. 233:74-83). In an embodiment, a guide RNA comprises ribonucleotides in a region that binds to a target sequence and one or more deoxyribonucletides and/or nucleotide analogs in a region that binds to the Cas5-HNH or Cas8-HNH polypeptide. In an embodiment, deoxyribonucleotides and/or nucleotide analogs are incorporated in engineered guide sequence structures. In one embodiment, 3-5 nucleotides at either the 3′ or the 5′ end of a guide sequence is chemically modified. In one embodiment, only minor modifications are introduced in the seed region, such as 2′-F modifications. In one embodiment, 2′-F modification is introduced at the 3′ end of a guide sequence. In an embodiment, three to five nucleotides at the 5′ and/or the 3′ end of the guide sequence are chemically modified with 2′-O-methyl (M), 2′-O-methyl 3′ phosphorothioate (MS), S-constrained ethyl (cEt), or 2′-O-methyl 3′ thioPACE (MSP). Such modification can enhance genome editing efficiency (see Hendel et al., Nat. Biotechnol. (2015) 33 (9): 985-989). In an embodiment, all the phosphodiester bonds of a guide sequence are substituted with phosphorothioates (PS) for enhancing levels of gene disruption. In an embodiment, more than five nucleotides at the 5′ and/or the 3′ end of the guide sequence are chemically modified with 2′-O-Me, 2′-F or S-constrained ethyl(cEt). Such chemically modified guide sequence can mediate enhanced levels of gene disruption (see Ragdarm et al., 0215, PNAS, E7110-E7111). In an embodiment of the invention, a guide sequence is modified to comprise a chemical moiety at its 3′ and/or 5′ end. Such moieties include, but are not limited to amine, azide, alkyne, thio, dibenzocyclooctyne (DBCO), or Rhodamine. In certain embodiments, the chemical moiety is conjugated to the guide sequence by a linker, such as an alkyl chain. In an embodiment, the chemical moiety of the modified guide sequence can be used to attach the guide sequence to another molecule, such as DNA, RNA, protein, or nanoparticles. Such chemically modified guide sequence can be used to identify or enrich cells generically edited by a Cas5-HNH or Cas8-HNH polypeptide nuclease and related systems (see Lee et al., eLife, 2017, 6:e25312, DOI: 10.7554).

In a particular embodiment, the conserved nucleotide sequence may be modified to comprise one or more protein-binding RNA aptamers. In a particular embodiment, one or more aptamers may be included such as part of optimized secondary structure. Such aptamers may be capable of binding a bacteriophage coat protein as detailed further herein.

The Cas5-HNH or Cas8-HNH polypeptide utilizes a guide sequence comprising a polynucleotide sequence that facilitates the interaction with the Cas5-HNH or Cas8-HNH polypeptide, allowing for sequence specific binding and/or targeting of the guide sequence with the target polynucleotide. Chemical synthesis of the guide sequence is contemplated, using covalent linkage using various bioconjugation reactions, loops, bridges, and non-nucleotide links via modifications of sugar, internucleotide phosphodiester bonds, purine and pyrimidine residues. Sletten et al., Angew. Chem. Int. Ed. (2009) 48:6974-6998; Manoharan, M. Curr. Opin. Chem. Biol. (2004) 8:570-9; Behlke et al., Oligonucleotides (2008) 18:305-19; Watts, et al., Drug. Discov. Today (2008) 13:842-55; Shukla, et al., ChemMedChem (2010) 5:328-49; chemical synthesis using automated, solid-phase oligonucleotide synthesis machines with 2′-acetoxyethyl orthoester (2′-ACE) (Scaringe et al., J. Am. Chem. Soc. (1998) 120:11820-11821; Scaringe, Methods Enzymol. (2000) 317:3-18) or 2′-thionocarbamate (2′-TC) chemistry (Dellinger et al., J. Am. Chem. Soc. (2011) 133:11540-11546; Hendel et al., Nat. Biotechnol. (2015) 33:985-989).

In certain example embodiments, the guide and spacer may be designed as two separate molecules that can hybridize or covalently joined into a single molecule. Covalent linkage can be via a linker (e.g., a non-nucleotide loop) that comprises a moiety such as spacers, attachments, bioconjugates, chromophores, reporter groups, dye labeled RNAs, and non-naturally occurring nucleotide analogues. More specifically, suitable spacers for purposes of this invention include, but are not limited to, polyethers (e.g., polyethylene glycols, polyalcohols, polypropylene glycol or mixtures of ethylene and propylene glycols), polyamines group (e.g., spennine, spermidine and polymeric derivatives thereof), polyesters (e.g., poly(ethyl acrylate)), polyphosphodiesters, alkylenes, and combinations thereof. Suitable attachments include any moiety that can be added to the linker to add additional properties to the linker, such as but not limited to, fluorescent labels. Suitable bioconjugates include, but are not limited to, peptides, glycosides, lipids, cholesterol, phospholipids, diacyl glycerols and dialkyl glycerols, fatty acids, hydrocarbons, enzyme substrates, steroids, biotin, digoxigenin, carbohydrates, polysaccharides. Suitable chromophores, reporter groups, and dye-labeled RNAs include, but are not limited to, fluorescent dyes such as fluorescein and rhodamine, chemiluminescent, electro-chemiluminescent, and bioluminescent marker compounds. The design of example linkers conjugating two RNA components are also described in WO 2004/015075.

The linker (e.g., a non-nucleotide loop) can be of any length. In one embodiment, the linker has a length equivalent to about 0-16 nucleotides. In one embodiment, the linker has a length equivalent to about 0-8 nucleotides. In one embodiment, the linker has a length equivalent to about 0-4 nucleotides. In one embodiment, the linker has a length equivalent to about 2 nucleotides. Example linker design is also described in International Patent Publication No. WO 2011/008730.

### Escorted Guides/Aptamers

In one embodiment, the compositions or complexes have a guide sequence with a functional structure designed to improve guide sequence structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (Levy-Nissenbaum, Etgar, et al. “Nanotechnology and aptamers: applications in drug delivery.” Trends in biotechnology 26.8 (2008): 442-449; and, Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (Zhou, Jichua, and John J. Rossi. “Aptamer-targeted cell-specific RNA interference.” Silence 1.1 (2010): 4).

Accordingly, in one embodiment, the guide sequence is modified, e.g., by one or more aptamer(s) designed to improve guide sequence delivery, including delivery across the cellular membrane, to intracellular compartments, or into the nucleus. Such a structure can include, either in addition to the one or more aptamer(s) or without such one or more aptamer(s), moiety(ies) so as to render the guide sequence deliverable, inducible or responsive to a selected effector. The invention accordingly comprehends a guide sequence that responds to normal or pathological physiological conditions, including without limitation pH, hypoxia, 02 concentration, temperature, protein concentration, enzymatic concentration, lipid structure, light exposure, mechanical disruption (e.g., ultrasound waves), magnetic fields, electric fields, or electromagnetic radiation.

Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

Energy sources such as electromagnetic radiation, sound energy or thermal energy may induce the guide sequence. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

The chemical or energy sensitive guide sequence may undergo a conformational change upon induction by the binding of a chemical source or by the energy allowing it act as a guide sequence and have the nucleic acid-guided nuclease system or complex function. The invention can involve applying the chemical source or energy so as to have the guide sequence function and the nucleic acid-guided nuclease system or complex function; and optionally further determining that the expression of the genomic locus is altered.

There are several different designs of this chemical inducible system: 1. ABI-PYL based system inducible by Abscisic Acid (ABA) (see, e.g., stke.sciencemag.org/cgi/content/abstract/sigtrans;4/164/rs2), 2. FKBP-FRB based system inducible by rapamycin (or related chemicals based on rapamycin) (see, e.g., www.nature.com/nmeth/journal/v2/n6/full/nmeth763.html), 3. GID1-GAI based system inducible by Gibberellin (GA) (see, e.g., www.nature.com/nchembio/journal/v8/n5/full/nchembio.922.html).

A chemical inducible system can be an estrogen receptor (ER) based system inducible by 4-hydroxytamoxifen (4OHT) (see, e.g., www.pnas.org/content/1Apr. 3, 1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel-based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the nucleic acid-guided nuclease/guide sequence complex or system, and the binding will induce the change of sub-cellular localization of the polypeptide, leading to the entire polypeptide entering the nucleus of cells. Once inside the nucleus, the guide protein and the other components of the nucleic acid-guided nuclease/guide sequence complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 us and 500 milliseconds, preferably between 1 us and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 k Volts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100. mu·s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably, the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably, the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz′ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see Morocz et al 1998 Journal of Magnetic Resonance Imaging Vol. 8, No. 1, pp. 136-142. Another form of focused ultrasound is high intensity focused ultrasound (HIFU) which is reviewed by Moussatov et al in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably, the exposure to an ultrasound energy source is at a power density of from about 0.05 to about 100 Wcm-2. Even more preferably, the exposure to an ultrasound energy source is at a power density of from about 1 to about 15 Wcm-2.

Preferably, the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably, the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In one embodiment, the guide sequence is modified by a secondary structure to increase the specificity of the nucleic acid-guided nuclease and related system and the secondary structure can protect against exonuclease activity and allow for 5′ additions to the guide sequence also referred to herein as a protected guide sequence.

In one aspect, the invention provides for hybridizing a “protector RNA” to a sequence of the guide sequence, wherein the “protector RNA” is an RNA strand complementary to the 3′ end of the guide molecule to thereby generate a partially double-stranded guide sequence. In an embodiment of the invention, protecting mismatched bases (i.e., the bases of the guide molecule which do not form part of the guide sequence) with a perfectly complementary protector sequence decreases the likelihood of target DNA binding to the mismatched base pairs at the 3′ end. In one embodiment of the invention, additional sequences comprising an extended length may also be present within the guide sequence such that the guide sequence comprises a protector sequence within the guide sequence. This “protector sequence” ensures that the guide sequence comprises a “protected sequence” in addition to an “exposed sequence” (comprising the part of the guide sequence hybridizing to the target sequence). In one embodiment, the guide sequence is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the nucleic acid-guided nuclease and related system interacting with its target. By providing such an extension including a partially double stranded guide sequence, the guide sequence is considered protected and results in improved specific binding of the nucleic acid-guided nuclease/guide sequence complex, while maintaining specific activity.

In one embodiment, use is made of a truncated guide sequence (tru-guide sequence), i.e., a guide sequence which comprises a guide sequence which is truncated in length with respect to the canonical guide sequence length. As described by Nowak et al. (Nucleic Acids Res (2016) 44 (20): 9555-9564), such guides may allow catalytically active nucleic acid-guided nuclease to bind its target without cleaving the target DNA. In one embodiment, a truncated guide sequence is used which allows the binding of the target but retains only nickase activity of the nucleic acid-guided nuclease.

In one embodiment, conjugation of triantennary N-acetyl galactosamine (GalNAc) to oligonucleotide components may be used to improve delivery, for example delivery to select cell types, for example hepatocytes (see International Patent Publication No. WO 2014/118272 incorporated herein by reference; Nair, J K et al., 2014, Journal of the American Chemical Society 136 (49), 16958-16961). This is considered to be a sugar-based particle and further details on other particle delivery systems and/or formulations are provided herein. GalNAc can therefore be considered a particle in the sense of the other particles described herein, such that general uses and other considerations, for instance delivery of said particles, apply to GalNAc particles as well. A solution-phase conjugation strategy may for example be used to attach triantennary GalNAc clusters (mol. wt. ˜2000) activated as PFP (pentafluorophenyl) esters onto 5′-hexylamino modified oligonucleotides (5′-HA ASOs, mol. wt. ˜8000 Da; Østergaard et al., Bioconjugate Chem., 2015, 26 (8), pp 1451-1455). Similarly, poly(acrylate) polymers have been described for in vivo nucleic acid delivery (see WO2013158141 incorporated herein by reference). In further alternative embodiments, pre-mixing nucleic acid-guided nuclease nanoparticles (or protein complexes) with naturally occurring serum proteins may be used in order to improve delivery (Akinc A et al, 2010, Molecular Therapy vol. 18 no. 7, 1357-1364).

Screening techniques are available to identify delivery enhancers, for example by screening chemical libraries (Gilleron J. et al., 2015, Nucl. Acids Res. 43 (16): 7984-8001). Approaches have also been described for assessing the efficiency of delivery vehicles, such as lipid nanoparticles, which may be employed to identify effective delivery vehicles for components (see Sahay G. et al., 2013, Nature Biotechnology 31, 653-658).

### PAM Specificity

In one example embodiment, the Cas5-HNH or Cas8-HNH polypeptide lack or substantially lack a PAM interacting (PI) domain. In an embodiment, the Cas5-HNH or Cas8-HNH polypeptide may have a PI domain or a functional fragment of a PI domain. In an embodiment, the Cas5-HNH or Cas8-HNH polypeptide may achieve a target specificity by a non-protein domain. In an embodiment, the nucleic acid-guided nucleases may have helicase activity. In an embodiment, the nucleic acid-guided nucleases may have reduced helicase activity compared to Cas proteins known in the art. In an embodiment, the nucleic acid-guided nucleases may comprise additional components that contribute in mediating target recognition. In an embodiment, targeting specificity is obtained by a central hairpin structure in a guide molecule.

Examples of PAM sequences for the Cas5-HNH polypeptide herein include the 5′-NNYNN-3′ where Y is A or T and N is any nucleotide. For example, the nucleic acid-guided nucleases may recognize PAM sequence NNAAN or NNATN, etc.

Examples of PAM sequences for the Cas8-HINH polypeptide herein include the 5′-NNYNN-3′ where Y is A, T, or G and Nis any nucleotide. For example, the nucleic acid-guided nucleases may recognize PAM sequence NNACN or NNTCN, etc. Examples of PAM sequences for the Cas8-HNH polypeptide herein include the 5′-NNNYN-3′ where Y is C and N is any nucleotide.

The PAM interaction domain or PI domain as referred to herein is reported to be responsible for determining PAM specificity of Cas5-HNH or Cas8-HNH polypeptides. By means of example, the PI domain is contained in the NUC lobe and forms an elongated structure comprising seven α-helices, a three-stranded antiparallel β-sheet, a five-stranded antiparallel β-sheet, and a two-stranded antiparallel β-sheet.

In some cases, where the nucleic acid-guided nucleases do have a PAM requirement, the precise sequence and length requirements for the PAM will differ depending on the nucleic acid-guided nucleases used. In some examples, PAMs are typically 2-5 base pair sequences adjacent the protospacer (that is, the target sequence). Examples of the natural PAM sequences for different nucleic acid-guided nucleases orthologs have been identified and the skilled person will be able to identify further PAM sequences for use with a given nucleic acid-guided nucleases.

Further, associating a PAM Interacting (PI) domain (e.g., attaching or fusing) to a nucleic acid-guided nuclease may allow programing of PAM specificity, improve target site recognition fidelity, and increase the versatility of the Cas5-HNH or Cas8-HNH polypeptide, genome engineering platform. nucleic acid-guided nucleases may be engineered to alter their PAM specificity, for example as described in Kleinstiver B P et al. Engineered CRISPR-Cas9 nucleases with altered PAM specificities. Nature. 2015 Jul. 23;523 (7561): 481-5. doi: 10.1038/nature14592. The skilled person will understand that other Cas5-HNH or Cas8-HNH polypeptides may be modified analogously.

The crystal structure information (described in U.S. Provisional Patent Application Nos. 61/915,251 filed Dec. 12, 2013, 61/930,214 filed on Jan. 22, 2014, 61/980,012 filed Apr. 15, 2014; and Nishimasu et al, “Crystal Structure of Cas9 in Complex with Guide RNA and Target DNA,” Cell 156 (5): 935-949, DOI: dx.doi.org/10.1016/j.cell.2014.02.001 (2014), each and all of which are incorporated herein by reference) provides structural information to truncate and create modular or multi-part CRISPR enzymes which may be incorporated into inducible composition. In particular, structural information is provided for S. pyogenes Cas9 (SpCas9), and this may be extrapolated to other Cas9 orthologs or Cas5-HNH or Cas8-HNH polypeptides (as well as homologs and orthologs thereof) or other nucleic acid-guided nucleases. In one embodiment, the conformational variations in the crystal structures of the CRISPR-Cas9 system or of components of the CRISPR-Cas9 provide important and critical information about the flexibility or movement of protein structure regions relative to nucleotide (RNA or DNA) structure regions that may be important for the function of other nucleic acid-guided nucleases and related systems. The structural information provided for Cas9 (e.g., S. pyogenes Cas9) as the nucleic acid-guided nuclease in the present application may be used to further engineer and optimize the other nucleic acid-guided nucleases and related system and this may be extrapolated to interrogate structure-function relationships in other nucleic acid-guided nucleases and related systems.

### HDR Donor Templates

In one embodiment, the compositions and systems herein may further comprise one or more nucleic acid templates. In some cases, the nucleic acid template may comprise one or more polynucleotides. In certain cases, the nucleic acid template may comprise coding sequences for one or more polynucleotides. The nucleic acid template may be a DNA template.

The donor polynucleotide may be used for editing the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild-type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In an embodiment of the invention, the donor polynucleotide may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may has a size from 10 base pair or nucleotides to 50 kb in length, e.g., from 50 to 40k, from 100 and 30 k, from 100 to 10000, from 100 to 300, from 200 to 400, from 300 to 500, from 400 to 600, from 500 to 700, from 600 to 800, from 700 to 900, from 800 to 1000, from 900 to from 1100, from 1000 to 1200, from 1100 to 1300, from 1200 to 1400, from 1300 to 1500, from 1400 to 1600, from 1500 to 1700, from 600 to 1800, from 1700 to 1900, from 1800 to 2000 base pairs (bp) or nucleotides in length.

### Protein Modifications

The Cas5-HNH or Cas8-HNH polypeptide herein include variants and mutated forms of Cas5 or Cas8 proteins (comparing to wildtype or naturally occurring Cas proteins). In some examples, the present disclosure includes variants and mutated forms of the Cas5 or Cas8 proteins. The Cas5-HNH or Cas8-HNH polypeptide may comprise one or more modifications. As used herein, the term “modified” with regard to a Cas5-HNH or Cas8-HNH polypeptide generally refers to a Cas5-HNH or Cas8-HNH polypeptide having one or more modifications or mutations (including point mutations, truncations, insertions, deletions, chimeras, fusion proteins, etc.) compared to the wild-type counterpart from which it is derived. By derived is meant that the derived enzyme is largely based, in the sense of having a high degree of sequence homology with, a wildtype enzyme, but that it has been mutated (modified) in some way as known in the art or as described herein.

The modified proteins, e.g., modified Cas5-HNH or Cas8-HNH polypeptide nuclease, may be catalytically inactive (also referred as dead). As used herein, a catalytically inactive or dead nuclease may have reduced, or no nuclease activity compared to a wildtype counterpart nuclease. In some cases, a catalytically inactive or dead nuclease may have nickase activity. In some cases, a catalytically inactive or dead nuclease may not have nickase activity. Such a catalytically inactive or dead nuclease may not make either double-strand or single-strand break on a target polynucleotide but may still bind or otherwise form complex with the target polynucleotide.

In an embodiment, the Cas5-HNH or Cas8-HNH polypeptide comprises one or more mutation in the HNH domain of the polypeptide of the polypeptide. In an embodiment, the Cas5-HNH or Cas8-HNH polypeptide comprises a mutation of the catalytic HINH residue corresponding to analogous mutations in other Cas proteins e.g., Cas9 or Cas12. Mutations to reduce or eliminate the HINH nucleolytic activity in other Cas proteins are known in the art and the analogous HNH mutations are envisioned herein.

In one embodiment, the modifications of the Cas5-HNH or Cas8-HNH polypeptide may or may not cause an altered functionality. By means of example, modifications which do not result in an altered functionality include for instance codon optimization for expression into a particular host, or providing the nuclease with a particular marker (e.g., for visualization). Modifications which may result in altered functionality may also include mutations, including point mutations, insertions, deletions, truncations (including split nucleases), etc., as well as chimeric nucleases (e.g., comprising domains from different orthologues or homologues) or fusion proteins. A chimeric enzyme can comprise a first fragment and a second fragment, and the fragments can be of Cas5-HNH or Cas8-HNH polypeptide orthologs of organisms of a genus or of a species, e.g., the fragments are from Cas5-HNH or Cas8-HNH polypeptide orthologs of different species. Fusion proteins may without limitation include, for instance, fusions with heterologous domains or functional domains (e.g., localization signals, catalytic domains, etc.). In an embodiment, various different modifications may be combined (e.g., a mutated nuclease which is catalytically inactive and which further is fused to a functional domain, such as for instance to induce DNA methylation or another nucleic acid modification, such as including without limitation, a break (e.g. by a different nuclease (domain)), a mutation, a deletion, an insertion, a replacement, a ligation, a digestion, a break or a recombination). As used herein, “altered functionality” includes without limitation an altered specificity (e.g., altered target recognition, increased (e.g., “enhanced” Cas5-HNH or Cas8-HNH polypeptide) or decreased specificity, or altered PAM recognition), altered activity (e.g., increased or decreased catalytic activity, including catalytically inactive nucleases or nickases), and/or altered stability (e.g., fusions with destabilization domains). Examples of all these modifications are known in the art. It will be understood that a “modified” nuclease as referred to herein, and in particular a “modified” Cas5-HNH or Cas8-HNH polypeptide or system or complex preferably still has the capacity to interact with or bind to the polynucleic acid (e.g., in complex with the guide sequence). Such modified Cas5-HNH or Cas8-HNH polypeptide can be combined with the deaminase protein or active domain thereof as described herein.

In one embodiment, unmodified Cas5-HNH or Cas8-HNH polypeptide may have cleavage activity. In one embodiment, the Cas5-HNH or Cas8-HNH polypeptide may direct cleavage of one or both DNA strands at the location of or near a target sequence, such as within the target sequence and/or within the complement of the target sequence or at sequences associated with the target sequence. In one embodiment, the Cas5-HNH or Cas8-HNH polypeptide may direct cleavage of one or both strands within about 1, 2, 3, 4, 5, 6, 7, 8, 9, 10, 15, 20, 25, 50, 100, 200, 500, or more base pairs or nucleotides from the first or last nucleotide of a target sequence. In one embodiment, the cleavage may be staggered, i.e., generating sticky ends. In one embodiment, the cleavage is a staggered cut with a 5′ overhang. In one embodiment, the cleavage is a staggered cut with a 5′ overhang of 1 to 15 nucleotides, preferably of 4 or 9 nucleotides. In example embodiments, the Cas5-HNH or Cas8-HNH polypeptide may direct more than one cleavage (such as one, two three, four, five, or more cleavages) of one or two strands within the target sequence and/or within the complement of the target sequence or at sequences associated with the target sequence and/or within about 1, 2, 3, 4, 5, 6, 7, 8, 9, 10, 15, 20, 25, 50, 100, 200, 500, or more base pairs from the first or last nucleotide of a target sequence. In example embodiments, the cleavage may be blunt, i.e., generating blunt ends. In example embodiments, the cleavage may be staggered, i.e., generating sticky ends.

In one embodiment, the cleavage site is distant from the Proto-Adjacent Motif (PAM), e.g., the cleavage occurs after the nth nucleotide on the non-target strand and after the nucleotide on the targeted strand. In one embodiment, the cleavage site occurs after an identified nucleotide (counted from the PAM) on the non-target strand and after the further identified nucleotide (counted from the PAM) on the targeted strand. In one embodiment, a vector encodes a nucleic acid-targeting effector protein that may be mutated with respect to a corresponding wild-type enzyme such that the mutated nucleic acid-targeting effector protein lacks the ability to cleave one or both DNA and RNA strands of a target polynucleotide containing a target sequence. As a further example, two or more catalytic domains of a Cas5-HNH or Cas8-HNH polypeptide (e.g., the HINH domain) may be mutated to produce a mutated Cas5-HNH or Cas8-HNH polypeptide substantially lacking all DNA cleavage activity. As described herein, corresponding catalytic domains of a Cas5-HNH or Cas8-HNH polypeptide may also be mutated to produce a mutated Cas5-HNH or Cas8-HNH polypeptide lacking all DNA cleavage activity or having substantially reduced DNA cleavage activity. In one embodiment, a Cas5-HNH or Cas8-HNH polypeptide may be considered to substantially lack all polynucleotide cleavage activity when the polynucleotide cleavage activity of the mutated enzyme is no more than 25%, no more than 10%, no more than 5%, no more than 1%, no more than 0.1%, no more than 0.01% of the nucleic acid cleavage activity of the non-mutated form of the enzyme; an example can be when the nucleic acid cleavage activity of the mutated form is nil or negligible as compared with the non-mutated form. A Cas5-HNH or Cas8-HNH polypeptide may be identified with reference to the general class of enzymes that share homology to the biggest nuclease with multiple nuclease domains from the Type I, II, III, IV, V, or VI CRISPR systems.

PAM identification and specificity may be identified, for example, using the methods disclosed in the Examples section below.

In an embodiment, the nuclease domains of the Cas5-HNH or Cas8-HNH polypeptide are catalytically inactive, or modified to be catalytically inactive, or when the protein is a nickase. In an embodiment, the nuclease domain is catalytically inactive, in which case the Cas5-HNH or Cas8-HINH polypeptide is referred to as dCas5-HNH or dCas8-HNH.

In an embodiment, the Cas5-HNH or Cas8-HNH polypeptide may comprise one or more modifications resulting in enhanced activity and/or specificity, such as including mutating residues that stabilize the targeted or non-targeted strand. In an embodiment, the altered or modified activity of the engineered Cas5-HNH or Cas8-HNH polypeptide comprises increased targeting efficiency or decreased off-target binding. In an embodiment, the altered activity of the engineered Cas5-HNH or Cas8-HNH polypeptide comprises modified cleavage activity. In an embodiment, the altered activity comprises increased cleavage activity as to the target polynucleotide loci. In an embodiment, the altered activity comprises decreased cleavage activity as to the target polynucleotide loci. In an embodiment, the altered activity comprises decreased cleavage activity as to off-target polynucleotide loci. In an embodiment, the altered or modified activity of the modified nuclease comprises altered helicase kinetics. In an embodiment, the modified nuclease comprises a modification that alters association of the protein with the nucleic acid molecule comprising RNA, or a strand of the target polynucleotide loci, or a strand of off-target polynucleotide loci. In an aspect of the invention, the engineered Cas5-HNH or Cas8-HNH polypeptide comprises a modification that alters formation of the Cas5-HNH or Cas8-HNH polypeptide and related complex. In an embodiment, the altered activity comprises increased cleavage activity as to off-target polynucleotide loci. Accordingly, in an embodiment, there is increased specificity for target polynucleotide loci as compared to off-target polynucleotide loci. In other embodiments, there is reduced specificity for target polynucleotide loci as compared to off-target polynucleotide loci. In an embodiment, the mutations result in decreased off-target effects (e.g., cleavage or binding properties, activity, or kinetics), such as in case for Cas5-HNH or Cas8-HNH polypeptide for instance resulting in a lower tolerance for mismatches between target and the guide sequences. Other mutations may lead to increased off-target effects (e.g., cleavage or binding properties, activity, or kinetics). Other mutations may lead to increased or decreased on-target effects (e.g., cleavage or binding properties, activity, or kinetics). In an embodiment, the mutations result in altered (e.g., increased or decreased) helicase activity, association or formation of the functional nuclease complex. In an embodiment, the mutations result in an altered PAM recognition, i.e., a different PAM may be (in addition or in the alternative) be recognized, compared to the unmodified Cas5-HNH or Cas8-HNH polypeptide. Examples mutations include positively charged residues and/or (evolutionary) conserved residues, such as conserved positively charged residues, in order to enhance specificity. In an embodiment, such residues may be mutated to uncharged residues, such as alanine.

In some cases, the present disclosure provides for mutated Cas proteins comprising one or more modified of amino acids. The amino acids: (a) interact with a guide RNA that forms a complex with the mutated Cas protein; (b) are in an active site, an inter-domain linker domain, or a bridge helix domain of the mutated Cas protein; or (c) a combination thereof.

The term “corresponding amino acid” or “residue which corresponds to” refers to a particular amino acid or analogue thereof in a Cas homolog or ortholog that is identical or functionally equivalent to an amino acid in reference Cas protein. Accordingly, as used herein, referral to an “amino acid position corresponding to amino acid position [X]” of a specified Cas protein represents referral to a collection of equivalent positions in other recognized Cas and structural homologues and families.

In certain embodiments, the specificity of the Cas5-HNH or Cas8-HNH polypeptide of the invention is altered or modified. It is to be understood that mutated Cas has an altered or modified specificity if the specificity is different than the specificity of the corresponding wild-type Cas (i.e., unmutated Cas). Specificity can be determined by means known in the art. By means of example, and without limitation, specificity can be determined by comparison of on-target activity and off-target activity. In certain embodiments, specificity is increased. In certain embodiments, specificity is increased by at least 5%, preferably at least 10%, more preferably at least 20%, such as at least 30%, at least 40%, at least 50%, at least 60%, at least 70%, at least 80%, at least 90%, or at least 100%. In certain embodiments, specificity is decreased. In certain embodiments, specificity is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the target binding of the Cas protein of the invention is altered or modified. It is to be understood that mutated Cas has an altered or modified target binding if the target binding is different than the target binding of the corresponding wild-type Cas (i.e., unmutated Cas). Target binding can be determined by means known in the art. By means of example, and without limitation, target binding can be determined by calculating binding strength or affinity (such as based on equilibrium constants, Ka, Kd, etc.). In certain embodiments, target binding is increased. In certain embodiments, target binding is increased by at least 5%, preferably at least 10%, more preferably at least 20%, such as at least 30%, at least 40%, at least 50%, at least 60%, at least 70%, at least 80%, at least 90%, or at least 100%. In certain embodiments, target binding is decreased. In certain embodiments, target binding is decreased by at least 5%, preferably at least 10%, more preferably at least 20%, such as at least 30%, at least 40%, at least 50%, at least 60%, at least 70%, at least 80%, at least 90%, or (substantially) 100%.

In certain embodiments, the off-target binding of the Cas protein of the invention is altered or modified. It is to be understood that mutated Cas has an altered or modified off-target binding if the off-target binding is different than the off-target binding of the corresponding wild-type Cas (i.e., unmutated Cas). Off-target binding can be determined by means known in the art. By means of example, and without limitation, off-target binding can be determined by calculating binding strength or affinity (such as based on equilibrium constants, Ka, Kd, etc.). In certain embodiments, off-target bindings increased. In certain embodiments, off-target binding is increased by at least 5%, preferably at least 10%, more preferably at least 20%, such as at least 30%, at least 40%, at least 50%, at least 60%, at least 70%, at least 80%, at least 90%, or at least 100%. In certain embodiments, off-target binding is decreased. In certain embodiments, off-target binding is decreased by at least 5%, preferably at least 10%, more preferably at least 20%, such as at least 30%, at least 40%, at least 50%, at least 60%, at least 70%, at least 80%, at least 90%, or (substantially) 100%.

The types of mutations in the Cas proteins can be conservative mutations or non-conservative mutations. In certain preferred embodiments, the amino acid which is mutated is mutated into alanine (A). In certain preferred embodiments, if the amino acid to be mutated is an aromatic amino acid, it is mutated into alanine or another aromatic amino acid (e.g., H, Y, W, or F). In certain preferred embodiments, if the amino acid to be mutated is a charged amino acid, it is mutated into alanine or another charged amino acid (e.g., H, K, R, D, or E). In certain preferred embodiments, if the amino acid to be mutated is a charged amino acid, it is mutated into alanine or another charged amino acid having the same charge. In certain preferred embodiments, if the amino acid to be mutated is a charged amino acid, it is mutated into alanine or another charged amino acid having the opposite charge.

The invention also provides for methods and compositions wherein one or more amino acid residues of the effector protein may be modified e.g., an engineered or non-naturally-occurring effector protein or Cas. In an embodiment, the modification may comprise mutation of one or more amino acid residues of the effector protein. The one or more mutations may be in one or more catalytically active domains of the effector protein, or a domain interacting with the crRNA (such as the guide sequence or direct repeat sequence). The effector protein may have reduced or abolished nuclease activity or alternatively increased nuclease activity compared with an effector protein lacking said one or more mutations. The effector protein may not direct cleavage of the RNA strand at the target locus of interest. In a preferred embodiment, the one or more mutations may comprise two mutations.

The Cas proteins herein may comprise one or more amino acids mutated. In example embodiments, the amino acid is mutated to A, P, or V, preferably A. In example embodiments, the amino acid is mutated to a hydrophobic amino acid. In example embodiments, the amino acid is mutated to an aromatic amino acid. In example embodiments, the amino acid is mutated to a charged amino acid. In example embodiments, the amino acid is mutated to a positively charged amino acid. In example embodiments, the amino acid is mutated to a negatively charged amino acid. In example embodiments, the amino acid is mutated to a polar amino acid. In example embodiments, the amino acid is mutated to an aliphatic amino acid.

In another aspect, the disclosure provides a mutated Cas protein comprising one or more mutations of amino acids, wherein the amino acids: interact with a guide RNA that forms a complex with the engineered Cas protein; or are in an active site, e.g., in HNH domain(s).

It will be appreciated that the effector protein is based on or derived from an enzyme, so the term ‘effector protein’ certainly includes ‘enzyme’ in example embodiments. However, it will also be appreciated that the effector protein may, as required in example embodiments, have DNA or RNA binding, but not necessarily cutting or nicking, activity, including a dead-Cas5-HNH or dead-Cas8-HNH protein function.

In one aspect, the invention provides a mutated Cas5-HNH or Cas8-HNH as described herein elsewhere, having one or more mutations resulting in reduced off-target effects, e.g., improved CRISPR enzymes for use in effecting modifications to target loci but which reduce or eliminate activity towards off-targets, such as when complexed to guide RNAs, as well as improved CRISPR enzymes for increasing the activity of CRISPR enzymes, such as when complexed with guide RNAs. It is to be understood that mutated enzymes as described herein below may be used in any of the methods according to the invention as described herein elsewhere. Any of the methods, products, compositions and uses as described herein elsewhere are equally applicable with the mutated CRISPR enzymes as further detailed below.

Slaymaker et al. recently described a method for the generation of Cas orthologues with enhanced specificity (Slaymaker et al. 2015 “Rationally engineered Cas nucleases with improved specificity”). This strategy can be used to enhance the specificity of the Cas protein. Primary residues for mutagenesis are preferably all positive charges residues within the HNH domain. Additional residues are positive charged residues that are conserved between different orthologues.

In an aspect, the invention also provides methods and mutations for modulating Cas5-HNH or Cas8-HNH binding activity and/or binding specificity. In certain embodiments, Cas5-HNH or Cas8-HNH proteins lacking nuclease activity are used. In certain embodiments, modified guide RNAs are employed that promote binding but not nuclease activity of a Cas5-HNH or Cas8-HNH nuclease. In such embodiments, on-target binding can be increased or decreased. Also, in such embodiments off-target binding can be increased or decreased. Moreover, there can be increased or decreased specificity as to on-target binding vs. off-target binding.

The methods and mutations which can be employed in various combinations to increase or decrease activity and/or specificity of on-target vs. off-target activity or increase or decrease binding and/or specificity of on-target vs. off-target binding, can be used to compensate or enhance mutations or modifications made to promote other effects. Such mutations or modifications made to promote other effects in include mutations or modification to the Cas5-HNH or Cas8-HNH and or mutation or modification made to a guide RNA. The methods and mutations of the invention are used to modulate Cas5-HNH or Cas8-HNH nuclease activity and/or binding with chemically modified guide RNAs.

In an aspect, the invention provides methods and mutations for modulating binding and/or binding specificity of Cas5-HNH or Cas8-HNH proteins according to the invention as defined herein comprising functional domains such as nucleases, transcriptional activators, transcriptional repressors, and the like. For example, a Cas5-HNH or Cas8-HNH polypeptide can be made nuclease-null, or having altered or reduced nuclease activity by introducing mutations such as for instance Cas5-HNH or Cas8-HNH mutations described herein elsewhere. Nuclease deficient Cas5-HNH or Cas8-HNH proteins are useful for RNA-guided target sequence dependent delivery of functional domains. The invention provides methods and mutations for modulating binding of Cas5-HNH or Cas8-HNH polypeptide. In one embodiment, the functional domain comprises VP64, providing an RNA-guided transcription factor. In another embodiment, the functional domain comprises Fok I, providing an RNA-guided nuclease activity. Mention is made of U.S. Pat. Pub. 2014/0356959, U.S. Pat. Pub. 2014/0342456, U.S. Pat. Pub. 2015/0031132, and Mali, P. et al., 2013, Science 339 (6121): 823-6, doi: 10.1126/science. 1232033, published online 3 Jan. 2013 and through the teachings herein the invention comprehends methods and materials of these documents applied in conjunction with the teachings herein. In certain embodiments, on-target binding is increased. In certain embodiments, off-target binding is decreased. In certain embodiments, on-target binding is decreased. In certain embodiments, off-target binding is increased. Accordingly, the invention also provides for increasing or decreasing specificity of on-target binding vs. off-target binding of functionalized Cas5-HNH or Cas8-HNH binding polypeptides. In example embodiments, the one or more functional domains is a transcriptional repressor domain. In example embodiments, the transcriptional repressor domain is a KRAB domain. In example embodiments, the transcriptional repressor domain is a NuE domain, NcoR domain, SID domain or a SID4X domain.

The use of Cas5-HNH or Cas8-HNH as an RNA-guided binding protein is not limited to nuclease-null Cas. Cas5-HNH or Cas8-HNH enzymes comprising nuclease activity can also function as RNA-guided binding proteins when used with certain guide RNAs. For example, short guide RNAs and guide RNAs comprising nucleotides mismatched to the target can promote RNA directed Cas5-HNH or Cas8-HNH binding to a target sequence with little or no target cleavage. (See, e.g., Dahlman, 2015, Nat Biotechnol. 33 (11): 1159-1161, doi: 10.1038/nbt.3390, published online 5 Oct. 2015). In an aspect, the invention provides methods and mutations for modulating binding of Cas5-HNH or Cas8-HNH proteins that comprise nuclease activity. In certain embodiments, on-target binding is increased. In certain embodiments, off-target binding is decreased. In certain embodiments, on-target binding is decreased. In certain embodiments, off-target binding is increased. In certain embodiments, there is increased or decreased specificity of on-target binding vs. off-target binding. In certain embodiments, nuclease activity of guide RNA-Cas5-HNH or guide RNA-Cas8-HNH enzyme is also modulated.

RNA-RNA duplex formation is important for cleavage activity and specificity throughout the target region, not only the seed region sequence closest to the PAM. Thus, truncated guide RNAs show reduced cleavage activity and specificity. In an aspect, the invention provides method and mutations for increasing activity and specificity of cleavage using altered guide RNAs.

In certain embodiments, the catalytic activity of the Cas5-HNH or Cas8-HNH polypeptide of the invention is altered or modified. It is to be understood that mutated Cas5-HNH or Cas8-HNH has an altered or modified catalytic activity if the catalytic activity is different than the catalytic activity of the corresponding wild-type Cas5-HNH or Cas8-HINH protein (e.g., unmutated Cas5-HNH or Cas8-HNH protein). Catalytic activity can be determined by means known in the art. By means of example, and without limitation, catalytic activity can be determined in vitro or in vivo by determination of indel percentage (for instance after a given time, or at a given dose). In certain embodiments, catalytic activity is increased. In certain embodiments, catalytic activity is increased by at least 5%, preferably at least 10%, more preferably at least 20%, such as at least 30%, at least 40%, at least 50%, at least 60%, at least 70%, at least 80%, at least 90%, or at least 100%. In certain embodiments, catalytic activity is decreased. In certain embodiments, catalytic activity is decreased by at least 5%, preferably at least 10%, more preferably at least 20%, such as at least 30%, at least 40%, at least 50%, at least 60%, at least 70%, at least 80%, at least 90%, or (substantially) 100%. The one or more mutations herein may inactivate the catalytic activity, which may substantially all catalytic activity, below detectable levels, or no measurable catalytic activity.

One or more characteristics of the engineered Cas5-HNH or Cas8-HNH polypeptide may be different from a corresponding wile type Cas5-HNH or Cas8-HNH protein. Examples of such characteristics include catalytic activity, gRNA binding, specificity of the Cas5-HNH or Cas8-HNH polypeptide (e.g., specificity of editing a defined target), stability of the Cas5-HNH or Cas8-HNH polypeptide, off-target binding, target binding, protease activity, nickase activity, PFS recognition. In some examples, an engineered Cas5-HNH or Cas8-HNH polypeptide may comprise one or more mutations of the corresponding wild-type Cas5-HNH or Cas8-HNH protein. In example embodiments, the catalytic activity of the engineered Cas5-HNH or Cas8-HNH polypeptide is increased as compared to a corresponding wildtype Cas5-HNH or Cas8-HNH protein. In example embodiments, the catalytic activity of the engineered Cas5-HNH or Cas8-HNH polypeptide is decreased as compared to a corresponding wildtype Cas5-HNH or Cas8-HNH protein. In example embodiments, the gRNA binding of the engineered Cas5-HNH or Cas8-HNH protein is increased as compared to a corresponding wildtype Cas5-HINH or Cas8-HNH protein. In example embodiments, the gRNA binding of the engineered Cas5-HNH or Cas8-HNH polypeptide is decreased as compared to a corresponding wildtype Cas5-HNH or Cas8-HNH protein. In example embodiments, the specificity of the Cas5-HNH or Cas8-HNH polypeptide is increased as compared to a corresponding wildtype Cas5-HINH or Cas8-HINH protein. In example embodiments, the specificity of the Cas5-HNH or Cas8-HNH polypeptide is decreased as compared to a corresponding wildtype Cas5-HNH or Cas8-HNH protein. In example embodiments, the stability of the Cas5-HNH or Cas8-HNH polypeptide is increased as compared to a corresponding wildtype Cas5-HNH or Cas8-HNH protein. In example embodiments, the stability of the Cas5-HNH or Cas8-HNH polypeptide is decreased as compared to a corresponding wildtype Cas5-HNH or Cas8-HNH protein. In example embodiments, the engineered Cas5-HNH or Cas8-HINH polypeptide further comprises one or more mutations which inactivate catalytic activity. In example embodiments, the off-target binding of the Cas5-HNH or Cas8-HNH polypeptide is increased as compared to a corresponding wildtype Cas5-HNH or Cas8-HNH protein. In example embodiments, the off-target binding of the Cas5-HNH or Cas8-HNH polypeptide is decreased as compared to a corresponding wildtype Cas5-HNH or Cas8-HNH protein. In example embodiments, the target binding of the Cas5-HNH or Cas8-HNH polypeptide is increased as compared to a corresponding wildtype Cas5-HNH or Cas8-HNH protein. In example embodiments, the target binding of the Cas5-HNH or Cas8-HNH polypeptide is decreased as compared to a corresponding wildtype Cas5-HNH or Cas8-HNH protein. In example embodiments, the engineered Cas5-HNH or Cas8-HNH polypeptide has a higher protease activity or polynucleotide-binding capability compared with a corresponding wildtype Cas5-HNH or Cas8-HNH protein. In example embodiments, the PFS recognition is altered as compared to a corresponding wildtype Cas5-HNH or Cas8-HNH protein.

In example embodiments, such as for Cas, a non-naturally occurring or engineered composition of the invention may comprise an accessory protein that enhances the Cas5-HNH or Cas8-HNH polypeptide activity. In such embodiments, the Cas5-HNH or Cas8-HNH polypeptide and the accessory protein may be from the same source or from a different source. In example embodiments, a non-naturally occurring or engineered composition of the invention comprises an accessory protein that represses Cas5-HNH or Cas8-HNH polypeptide activity. In example embodiments, a non-naturally occurring or engineered composition of the invention comprises two or more crRNAs. In example embodiments, a non-naturally occurring or engineered composition of the invention comprises a guide sequence that hybridizes to a target RNA sequence in a prokaryotic cell. In example embodiments, a non-naturally occurring or engineered composition of the invention comprises a guide sequence that hybridizes to a target RNA sequence in a eukaryotic cell. In some embodiment, the Cas5-HNH or Cas8-HNH polypeptide comprises one or more nuclear localization signals (NLSs).

In some embodiment of the non-naturally occurring or engineered composition of the invention, the Cas5-HNH or Cas8-HNH protein and the accessory protein are from the same organism.

In some embodiment of the non-naturally occurring or engineered composition of the invention, the Cas5-HNH or Cas8-HNH protein and the accessory protein are from different organisms.

In certain embodiments, the Cas5-HNH or Cas8-HNH proteins herein may be associated with a locus comprising short CRISPR repeats between 30 and 40 bp long, more typically between 34 and 38 bp long, even more typically between 36 and 37 bp long, e.g., 30, 31, 32, 33, 34, 35, 36, 37, 38, 39, or 40 bp long. In certain embodiments the CRISPR repeats are long or dual repeats between 80 and 350 bp long such as between 80 and 200 bp long, even more typically between 86 and 88 bp long, e.g., 80, 81, 82, 83, 84, 85, 86, 87, 88, 89, or 90 bp long.

In certain embodiments of the invention, the guide RNA or mature crRNA comprises, consists essentially of, or consists of a direct repeat sequence and a guide sequence or spacer sequence. In certain embodiments, the guide RNA or mature crRNA comprises, consists essentially of, or consists of a direct repeat sequence linked to a guide sequence or spacer sequence. In preferred embodiments of the invention, the mature crRNA comprises a stem loop or an optimized stem loop structure or an optimized secondary structure. In preferred embodiments, the mature crRNA comprises a stem loop or an optimized stem loop structure in the direct repeat sequence, wherein the stem loop or optimized stem loop structure is important for cleavage activity. In certain embodiments, the mature crRNA preferably comprises a single stem loop. In certain embodiments, the direct repeat sequence preferably comprises a single stem loop. In certain embodiments, the cleavage activity of the effector protein complex is modified by introducing mutations that affect the stem loop RNA duplex structure. In preferred embodiments, mutations which maintain the RNA duplex of the stem loop may be introduced, whereby the cleavage activity of the effector protein complex is maintained. In other preferred embodiments, mutations which disrupt the RNA duplex structure of the stem loop may be introduced, whereby the cleavage activity of the effector protein complex is completely abolished.

The CRISPR system as provided herein can make use of a crRNA or analogous polynucleotide comprising a guide sequence, wherein the polynucleotide is an RNA, a DNA or a mixture of RNA and DNA, and/or wherein the polynucleotide comprises one or more nucleotide analogs. The sequence can comprise any structure, including but not limited to a structure of a native crRNA, such as a bulge, a hairpin or a stem loop structure. In certain embodiments, the polynucleotide comprising the guide sequence forms a duplex with a second polynucleotide sequence which can be an RNA or a DNA sequence.

### Nuclear Localization Sequences

In one embodiment, the Cas5-HNH or Cas8-HNH polypeptide nuclease is fused to one or more nuclear localization sequences (NLSs), such as about or more than about 1, 2, 3, 4, 5, 6, 7, 8, 9, 10, or more NLSs. In one embodiment, the Cas5-HNH or Cas8-HNH polypeptide nuclease comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. zero or at least one or more NLS at the amino-terminus and zero or at one or more NLS at the carboxy terminus).

When more than one NLS is present, each may be selected independently of the others, such that a single NLS may be present in more than one copy and/or in combination with one or more other NLSs present in one or more copies. In a preferred embodiment of the invention, the Nucleic acid-guided nuclease comprises at most 6 NLSs. In a preferred embodiment of the invention, the Cas5-HNH or Cas8-HNH polypeptide nuclease comprises at most 6 NLSs.

In one embodiment, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 14); the NLS from nucleoplasmin (e.g. the nucleoplasmin bipartite NLS with the sequence KRPAATKKAGQAKKKK (SEQ ID NO: 15); the c-myc NLS having the amino acid sequence PAAKRVKLD (SEQ ID NO: 16) or RQRRNELKRSP (SEQ ID NO: 17); the hRNPA1 M9 NLS having the sequence NQSSNFGPMKGGNFGGRSSGPYGGGGQYFAKPRNQGGY (SEQ ID NO: 18); the sequence RMRIZFKNKGKDTAELRRRRVEVSVELRKAKKDEQILKRRNV (SEQ ID NO: 19) of the IBB domain from importin-alpha; the sequences VSRKRPRP (SEQ ID NO: 20) and PPKKARED (SEQ ID NO: 21) of the myoma T protein; the sequence PQPKKKPL (SEQ ID NO: 22) of human p53; the sequence SALIKKKKKMAP (SEQ ID NO: 23) of mouse c-abl IV; the sequences DRLRR (SEQ ID NO: 24) and PKQKKRK (SEQ ID NO: 25) of the influenza virus NS1; the sequence RKLKKKIKKL (SEQ ID NO: 26) of the Hepatitis virus delta antigen; the sequence REKKKFLKRR (SEQ ID NO: 27) of the mouse Mx1 protein; the sequence KRKGDEVDGVDEVAKKKSKK (SEQ ID NO: 28) of the human poly(ADP-ribose) polymerase; and the sequence RKCLQAGMNLEARKTKK (SEQ ID NO: 29) of the steroid hormone receptors (human) glucocorticoid.

In general, the one or more NLSs are of sufficient strength to drive accumulation of the nucleic acid-guided nuclease in a detectable amount in the nucleus of a eukaryotic cell. In general, strength of nuclear localization activity may derive from the number of NLSs in the nucleic acid-guided nuclease, the particular NLS(s) used, or a combination of these factors. Detection of accumulation in the nucleus may be performed by any suitable technique. For example, a detectable marker may be fused to the nucleic acid-guided nuclease, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of complex formation (e.g., assay for DNA cleavage or mutation at the target sequence, or assay for altered gene expression activity affected by complex formation and/or nucleic acid-guided nuclease activity), as compared to a control no exposed to the nucleic acid-guided nuclease or complex, or exposed to a nucleic acid-guided nuclease lacking the one or more NLSs. In an embodiment of the herein described nucleic acid-guided nuclease protein complexes and systems the codon optimized nucleic acid-guided nuclease proteins comprise an NLS attached to the C-terminal of the protein. In an embodiment, other localization tags may be fused to the nucleic acid-guided nuclease, such as without limitation for localizing the nucleic acid-guided nuclease to particular sites in a cell, such as organelles, such as mitochondria, plastids, chloroplast, vesicles, golgi, (nuclear or cellular) membranes, ribosomes, nucleolus, ER, cytoskeleton, vacuoles, centrosome, nucleosome, granules, centrioles, etc.

In an embodiment of the invention, at least one nuclear localization signal (NLS) is attached to the nucleic acid sequences encoding the Cas5-HNH or Cas8-HNH polypeptide nuclease. In preferred embodiments at least one or more C-terminal or N-terminal NLSs are attached (and hence nucleic acid molecule(s) coding for the Cas5-HNH or Cas8-HNH polypeptide nuclease can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

### Linkers

In an embodiment of the invention, at least one nuclear localization signal (NLS) is attached to the nucleic acid sequences encoding the nucleic acid-guided nuclease or the Cas5-HNH or Cas8-HNH polypeptide. In preferred embodiments at least one or more C-terminal or N-terminal NLSs are attached (and hence nucleic acid molecule(s) coding for the nucleic acid-guided nuclease or Cas5-HNH or Cas8-HNH polypeptide nuclease can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

In some preferred embodiments, the functional domain is linked to a Cas5-HNH or Cas8-HNH polypeptide nuclease (e.g., an active or a dead Cas5-HNH or Cas8-HNH polypeptide nuclease) to target and activate epigenomic sequences such as promoters or enhancers. One or more guides directed to such promoters or enhancers may also be provided to direct the binding of the Cas5-HNH or Cas8-HNH polypeptide nuclease to such promoters or enhancers.

The term “associated with” is used here in relation to the association of the functional domain to the Cas5-HNH or Cas8-HNH polypeptide nuclease protein, nucleic acid-guided nuclease, or the adaptor protein. It is used in respect of how one molecule ‘associates’ with respect to another, for example between an adaptor protein and a functional domain, between the Cas5-HNH or Cas8-HNH polypeptide nuclease protein and a functional domain, or between the nucleic acid guided nuclease protein and a functional domain. In the case of such protein-protein interactions, this association may be viewed in terms of recognition in the way an antibody recognizes an epitope. Alternatively, one protein may be associated with another protein via a fusion of the two, for instance one subunit being fused to another subunit. Fusion typically occurs by addition of the amino acid sequence of one to that of the other, for instance via splicing together of the nucleotide sequences that encode each protein or subunit. Alternatively, this may essentially be viewed as binding between two molecules or direct linkage, such as a fusion protein. In any event, the fusion protein may include a linker between the two subunits of interest (i.e., between the enzyme and the functional domain or between the adaptor protein and the functional domain). Thus, in one embodiment, the Cas5-HNH or Cas8-HNH polypeptide nuclease protein, nucleic acid-guided nuclease, or adaptor protein is associated with a functional domain by binding thereto. In other embodiments, the Cas5-HNH or Cas8-HNH polypeptide nuclease, nucleic acid-guided nuclease, or adaptor protein is associated with a functional domain because the two are fused together, optionally via an intermediate linker.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in an embodiment, the linker may be selected to influence some property of the linker and/or the fusion protein such as the folding, net charge, or hydrophobicity of the linker.

Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond).

In one embodiment, the linker is used to separate the Cas5-HNH or Cas8-HNH polypeptide nuclease and the nucleotide deaminase by a distance sufficient to ensure that each protein retains its required functional property. In one embodiment, the linker is used to separate the nucleic acid-guided nuclease and the nucleotide deaminase by a distance sufficient to ensure that each protein retains its required functional property.

Preferred peptide linker sequences adopt a flexible extended conformation and do not exhibit a propensity for developing an ordered secondary structure. In an embodiment, the linker can be a chemical moiety which can be monomeric, dimeric, multimeric or polymeric. Preferably, the linker comprises amino acids. Typical amino acids in flexible linkers include Gly, Asn and Ser. Accordingly, in one embodiment, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40:39-46; Murphy et al. (1986) Proc. Nat'l. Acad. Sci. USA 83:8258-62; U.S. Pat. Nos. 4,935,233; and 4,751,180. For example, GlySer linkers GGS, GGGS (SEQ ID NO: 30) or GSG can be used. GGS, GSG, GGGS (SEQ ID NO: 30) or GGGGS (SEQ ID NO: 31) linkers can be used in repeats of 3 (such as (GGS)₃, (SEQ ID NO: 32) (GGGGS)₃) (SEQ ID NO: 33) or 5, 6, 7, 9 or even 12 or more, to provide suitable lengths. In some cases, the linker may be (GGGGS)₃₋₁₅, For example, in some cases, the linker may be (GGGGS)₃₋₁₁, e.g., GGGGS (SEQ ID NO: 31), (GGGGS)₂ (SEQ ID NO: 34), (GGGGS)₃ (SEQ ID NO: 33), (GGGGS)₄ (SEQ ID NO: 35), (GGGGS)₅ (SEQ ID NO: 36), (GGGGS)₆ (SEQ ID NO: 37), (GGGGS)₇ (SEQ ID NO: 38), (GGGGS): (SEQ ID NO: 39), (GGGGS)₉ (SEQ ID NO: 40), (GGGGS)₁₀ (SEQ ID NO: 41), or (GGGGS)₁₁ (SEQ ID NO: 42).

In one embodiment, linkers such as (GGGGS)₃ (SEQ ID NO: 33) are preferably used herein. (GGGGS) (SEQ ID NO: 37), (GGGGS)₉ (SEQ ID NO: 40) or (GGGGS)₁₂ (SEQ ID NO: 42) may preferably be used as alternatives. Other preferred alternatives are (GGGGS)₁ (SEQ ID NO: 43, (GGGGS)₂ (SEQ ID NO: 34), (GGGGS)₄ (SEQ ID NO: 35), (GGGGS)₅ (SEQ ID NO: 36), (GGGGS)₇ (SEQ ID NO: 38), (GGGGS): (SEQ ID NO: 39), (GGGGS)₁₀ (SEQ ID NO: 41), or (GGGGS)₁₁ (SEQ ID NO: 42). In yet a further embodiment, LEPGEKPYKCPECGKSFSQSGALTRHQRTHTR (SEQ ID NO: 44) is used as a linker. In yet an additional embodiment, the linker is an XTEN linker. In one embodiment, the Cas12b polypeptide nuclease or the nucleic acid-guided nuclease is linked to the deaminase protein or its catalytic domain by means of an LEPGEKPYKCPECGKSFSQSGALTRHQRTHTR (SEQ ID NO: 44) linker. In further one embodiment, Cas12b polypeptide nuclease is linked C-terminally to the N-terminus of a deaminase protein or its catalytic domain by means of an LEPGEKPYKCPECGKSFSQSGALTRHQRTHTR (SEQ ID NO: 44) linker. In addition, N- and C-terminal NLSs can also function as linker (e.g., PKKKRKVEASSPKKRKVEAS (SEQ ID NO: 45)).

### Examples of Linkers Used in the Invention are Shown

| | |
|---|---|
| GGS | GGTGGTAGT |
| GGSx3 (9) (SEQ | GGTGGTAGTGGAGGGAGCGGCGGTTCA (SEQ ID NO: 47) |
| ID NO: 32) | |
| GGSx7 (21) | ggtggaggaggctctggtggaggcggtagcggaggcggagggtcgGGTGGTAGTGGAGGGAGCGGCGGT |
| (SEQ ID NO: 46) | TCA (SEQ ID NO: 48) |
| XTEN | TCGGGATCTGAGACGCCTGGGACCTCGGAATCGGCTACGCCCGAAAGT (SEQ ID |
| | NO: 49) |
| Z-EGFR Short | Gtggataacaaatttaacaaagaaatgtgggggcgtgggaagaaattcgtaacctgccgaacctgaacggctgg |
| | cagatgaccgcgtttattgcgagcctggtggatgatccgagccagagcgcgaacctgctggcggaagcgaaaaa |
| | actgaacgatgcgcaggcgccgaaaaccggcggtggttctggt (SEQ ID NO: 50) |
| GSAT | Ggtggttctgccggtggctccggttctggctccagcggtggcagctctggtgcgtccggcacgggtactgcggg |
| | ggcactggcagcggttccggtactggctctggc (SEQ ID NO: 51) |

Linkers may be used between the guide or scaffold sequences and the functional domain (activator or repressor), or between the Cas5-HNH or Cas8-HNH polypeptide nuclease and the functional domain. In an embodiment, linkers may be used between the guide molecules and the functional domain (e.g., activator or repressor), or between the Cas5-HNH or Cas8-HNH polypeptide and the functional domain. The linkers may be used to engineer appropriate amounts of “mechanical flexibility.” In an embodiment, the one or more functional domains are controllable, e.g., inducible.

Deactivated/Inactivated/Dead Cas proteins

In certain embodiments, the Cas5-HNH or Cas8-HNH polypeptide herein is a catalytically inactive or dead Cas5-HNH or Cas8-HNH protein. In some cases, the Cas5-HNH or Cas8-HNH polypeptide herein is a catalytically inactive or dead Cas5-HNH or Cas8-HNH protein (dCas5-HNH or dCas8-HNH). In some cases, a dead Cas5-HNH or dead Cas8-HINH protein, e.g., a dead Cas5-HNH or dead Cas8-HNH protein has nickase activity. In example embodiments, the dCas5-HNH or dCas8-HNH protein comprises mutations in the nuclease domain. In example embodiments, the dCas5-HNH or dCas8-HNH protein has been truncated. In some cases, the dead Cas proteins may be fused with a deaminase herein, e.g., an adenosine deaminase.

Where the Cas5-HNH or Cas8-HNH protein has nuclease activity, the Cas5-HNH or Cas8-HNH protein may be modified to have diminished nuclease activity e.g., nuclease inactivation of at least 70%, at least 80%, at least 90%, at least 95%, at least 97%, or 100% as compared with the wild-type enzyme; or to put in another way, a Cas5-HNH or Cas8-HNH enzyme having advantageously about 0% of the nuclease activity of the non-mutated or wild-type Cas5-HNH or Cas8-HNH, or no more than about 3% or about 5% or about 10% of the nuclease activity of the non-mutated or wild-type Cas5-HNH or Cas8-HNH. This is possible by introducing mutations into the nuclease domains of the Cas5-HNH or Cas8-HNH and orthologs thereof.

The inactivated Cas CRISPR enzyme may have associated (e.g., via fusion protein) one or more functional domains, including for example, one or more domains from the group comprising, consisting essentially of, or consisting of methylase activity, demethylase activity, transcription activation activity, transcription repression activity, transcription release factor activity, histone modification activity, RNA cleavage activity, DNA cleavage activity, nucleic acid binding activity, molecular switches (e.g., light inducible), or any other functional domain described herein. Preferred domains are Fok1, VP64, P65, HSF1, MyoD1. In the event that Fok1 is provided, it is advantageous that multiple Fok1 functional domains are provided to allow for a functional dimer and that gRNAs are designed to provide proper spacing for functional use (Fok1) as specifically described in Tsai et al. Nature Biotechnology, Vol. 32, Number 6, June 2014). The adaptor protein may utilize known linkers to attach such functional domains. In some cases, it is advantageous that additionally at least one NLS is provided. In some instances, it is advantageous to position the NLS at the N terminus. When more than one functional domain is included, the functional domains may be the same or different.

In general, the positioning of the one or more functional domain on the inactivated Cas enzyme is one which allows for correct spatial orientation for the functional domain to affect the target with the attributed functional effect. For example, if the functional domain is a transcription activator (e.g., VP64 or p65), the transcription activator is placed in a spatial orientation which allows it to affect the transcription of the target. Likewise, a transcription repressor will be advantageously positioned to affect the transcription of the target, and a nuclease (e.g., Fok1) will be advantageously positioned to cleave or partially cleave the target. This may include positions other than the N-/C-terminus of the CRISPR enzyme. See, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32 (6): 569-77 (2014), relates to dimeric RNA-guided FokI Nucleases that recognize extended sequences and can edit endogenous genes with high efficiencies in human cells.

The dead or deactivated Cas proteins may be used as target-binding proteins, (e.g., DNA binding proteins). In these cases, the dead or deactivated Cas proteins may be fused with one or more functional domains.

As described herein, corresponding catalytic domains of a Cas5-HNH or Cas8-HNH effector protein may also be mutated to produce a mutated Cas5-HNH or Cas8-HNH effector protein lacking all DNA cleavage activity or having substantially reduced DNA cleavage activity. In example embodiments, a nucleic acid-targeting effector protein may be considered to substantially lack all RNA cleavage activity when the RNA cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. An effector protein may be identified with reference to the general class of enzymes that share homology to the biggest nuclease with multiple nuclease domains from the Type I CRISPR system. In further embodiments, the effector protein is a Type I protein. By derived, Applicants mean that the derived enzyme is largely based, in the sense of having a high degree of sequence homology with, a wildtype enzyme, but that it has been mutated (modified) in some way as known in the art or as described herein.

### Functional Domains Modifications

The Cas5-HNH or Cas8-HNH polypeptide (including variants such as a catalytically inactive form) may be associated with one or more functional domains (e.g., via fusion protein or suitable linkers). In an embodiment, the Cas5-HNH or Cas8-HNH polypeptide, or an ortholog or homolog thereof, may be used as a generic nucleic acid binding protein with fusion to or being operably linked to one or more functional domains. In an example embodiment, the functional domain is a recombinase. In one example, the functional domain is a deaminase. In another example, the functional domain is a transposase. In another example, the functional domain is a reverse transcriptase. In some cases, a functional domain may be associate with (e.g., fuse to) the Cas5-HNH or Cas8-HNH polypeptide. In some cases, a functional domain may be a protein different from the Cas5-HNH or Cas8-HNH polypeptide. In such cases, a functional domain and the Cas5-HNH or Cas8-HNH polypeptide may form a protein complex.

It is also envisaged that the Cas5-HNH or Cas8-HNH polypeptide-guide RNA molecule complex as a whole may be associated with two or more functional domains. For example, there may be two or more functional domains associated with the Cas5-HNH or Cas8-HNH polypeptide, or there may be one or more functional domains associated with the RNA-targeting effector protein and one or more functional domains associated with the guide RNA molecule (via one or more adaptor proteins).

In one embodiment, the Cas5-HNH or Cas8-HNH polypeptide is associated with one or more functional domains. The association can be by direct linkage of the effector protein to the functional domain, or by association with the crRNA. In a non-limiting example, the crRNA comprises an added or inserted sequence that can be associated with a functional domain of interest, including, for example, an aptamer or a nucleotide that binds to a nucleic acid binding adapter protein. The functional domain may be a functional heterologous domain.

It will be appreciated that any of the functionalities described herein may be engineered into Cas polypeptide from other orthologs, including chimeric enzymes comprising fragments from multiple orthologs. A chimeric enzyme can comprise a first fragment and a second fragment, and the fragments can be of CRISPR enzyme orthologs of organisms of genera herein mentioned or of species herein mentioned; advantageously the fragments are from CRISPR enzyme orthologs of different species.

In one embodiment, the invention also provides for the one or more heterologous functional domains to have one or more of the following activities: transposase activity, methylase activity, demethylase activity, translation activation activity, translation repression activity, transcription activation activity, transcription repression activity, transcription release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity and nucleic acid binding activity, detectable activity, or any combination thereof. Functional domain activity refers to a functional domain capable of performing the action of said activity. For example, a function domain having transposase activity refers to a functional domain comprising a composition capable of performing a transposition. At least one or more heterologous functional domains may be at or near the amino-terminus of the effector protein and/or wherein at least one or more heterologous functional domains is at or near the carboxy-terminus of the effector protein. The one or more heterologous functional domains may be fused to the effector protein. The one or more heterologous functional domains may be tethered to the effector protein. The one or more heterologous functional domains may be linked to the effector protein by a linker moiety.

In an embodiment, the Cas5-HNH or Cas8-HNH polypeptide or an ortholog or homolog thereof, may be used as a generic nucleic acid binding protein with fusion to or being operably linked to a functional domain. Exemplary functional domains may include but are not limited to translational initiator, translational activator, translational repressor, nucleases, in particular ribonucleases, a spliceosome, beads, a light inducible/controllable domain or a chemically inducible/controllable domain. In an embodiment, the one or more functional domains are controllable, e.g., inducible.

In one embodiment, one or more functional domains are associated with a Cas5-HNH or Cas8-HNH polypeptide via an adaptor protein, for example as used with the modified guides of Konnerman et al. (Nature 517, 583-588, 29 Jan. 2015).

In example embodiments, the one or more functional domains is attached to the Cas polypeptide so that upon binding to the sgRNA and target the functional domain is in a spatial orientation allowing for the functional domain to function in its attributed function.

In one embodiment, the one or more functional domains is attached to the adaptor protein so that upon binding of the Cas5-HNH or Cas8-HNH polypeptide to the guide RNA molecule and target, the functional domain is in a spatial orientation allowing for the functional domain to function in its attributed function.

In example embodiments, one or more functional domains are associated with a dead gRNA (dRNA). In example embodiments, a dRNA complex with active Cas protein directs gene regulation by a functional domain at on gene locus while an gRNA directs DNA cleavage by the active Cas protein at another locus, for example as described analogously in CRISPR-Cas systems by Dahlman et al., ‘Orthogonal gene control with a catalytically active Cas9 nuclease’. In example embodiments, dRNAs are selected to maximize selectivity of regulation for a gene locus of interest compared to off-target regulation. In example embodiments, dRNAs are selected to maximize target gene regulation and minimize target cleavage.

For the purposes of the following discussion, reference to a functional domain could be a functional domain associated with the Cas protein or a functional domain associated with the adaptor protein.

In the practice of the invention, loops of the gRNA may be extended, without colliding with the Cas protein by the insertion of distinct RNA loop(s) or distinct sequence(s) that may recruit adaptor proteins that can bind to the distinct RNA loop(s) or distinct sequence(s).

The adaptor proteins may include but are not limited to orthogonal RNA-binding protein/aptamer combinations that exist within the diversity of bacteriophage coat proteins. A list of such coat proteins includes, but is not limited to: Qβ, F2, GA, fr, JP501, M12, R17, BZ13, JP34, JP500, KU1, M11, MX1, TW18, VK, SP, FI, ID2, NL95, TW19, AP205, ϕCb5, ϕCb8r, ϕCb12r, ϕCb23r, 7s and PRR1. These adaptor proteins or orthogonal RNA binding proteins can further recruit effector proteins or fusions which comprise one or more functional domains.

Examples of functional domains include deaminase domain, transposase domain (e.g. helitron), reverse transcriptase domain, integrase domain, recombinase domain, resolvase domain, invertase domain, protease domain, DNA methyltransferase domain, DNA hydroxylmethylase domain, RNA polymerase domains, DNA demethylase domain, histone acetylase domain, histone deacetylases domain, nuclease domain (e.g. VirD2 domain), repressor domain, activator domain, nuclear-localization signal domains, transcription-regulatory protein (or transcription complex recruiting) domain, cellular uptake activity associated domain, nucleic acid binding domain, antibody presentation domain, histone modifying enzymes, recruiter of histone modifying enzymes; inhibitor of histone modifying enzymes, histone methyltransferase, histone demethylase, histone kinase, histone phosphatase, histone ribosylase, histone deribosylase, histone ubiquitinase, histone deubiquitinase, histone biotinase and histone tail protease. In some preferred embodiments, the functional domain is a transcriptional activation domain, such as, without limitation, VP64, p65, MyoD1, HSF1, RTA, SET7/9 or a histone acetyltransferase. In one embodiment, the functional domain is a transcription repression domain, preferably KRAB. In one embodiment, the transcription repression domain is SID, or concatemers of SID (e.g., SID4X). In one embodiment, the functional domain is an epigenetic modifying domain, such that an epigenetic modifying enzyme is provided. In one embodiment, the functional domain is an activation domain, which may be the P65 activation domain.

### Epigenetic Editing Systems

In one aspect of the invention, is provided a fusion protein comprising from N-terminus to C-terminus, a demethylation domain, an XTEN linker, and a nuclease-deficient RNA-guided DNA endonuclease enzyme or a nuclease-deficient endonuclease enzyme. In aspects, the fusion protein further comprises a transcriptional activator. In aspects, the transcriptional activator is VP64, p65, Rta, or a combination of two or more thereof. In another aspect, the fusion protein further comprises a nuclear localization sequence. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present invention provides a fusion protein comprising from N-terminus to C-terminus, an RNA-binding sequence, an XTEN linker, and a transcriptional activator. In aspects, the transcriptional activator is VP64, p65, Rta, or a combination of two or more thereof. In aspects, the fusion protein further comprises a demethylation domain, a nuclease-deficient RNA-guided DNA endonuclease enzyme or a nuclease-deficient endonuclease enzyme, a nuclear localization sequence, or a combination of two or more thereof. In embodiments, the fusion protein comprises the nuclease-deficient RNA-guided DNA endonuclease enzyme. In embodiments, the fusion protein comprises the nuclease-deficient DNA endonuclease enzyme.

In certain embodiments, the present invention provides a method of activating a target nucleic acid sequence in a cell, the method comprising: (i) delivering a first polynucleotide encoding a fusion protein described herein including embodiments thereof to a cell containing the silenced target nucleic acid; and (ii) delivering to the cell a second polynucleotide comprising: (a) a sgRNA or (b) a cr:tracrRNA; thereby reactivating the silenced target nucleic acid sequence in the cell. In aspects, the sgRNA comprises at least one MS2 stem loop. In aspects, the second polynucleotide comprises a transcriptional activator. In aspects, the second polynucleotide comprises two or more sgRNA.

In certain embodiments, the present invention provides a method of screening for one or more genetic elements that modulate expression of the METTL17 gene, the method comprising: contacting a plurality of cells with a library of structurally distinct small guide RNAs (sgRNAs) that target a plurality of genetic elements, thereby generating a plurality of test cells, the plurality of test cells each comprising: a small guide RNA (sgRNA); and a nuclease deficient sgRNA-mediated nuclease (dCas9), wherein the dCas9 comprises a dCas9 domain fused to a transcriptional modulator; or a dCas9 domain fused to an epitope fusion domain, selecting the test cells on the basis of the phenotype; and quantitating the frequency of the structurally distinct sgRNAs within the population of selected cells, wherein the sgRNAs that target genetic elements that modulate the phenotype are overrepresented or underrepresented in the selected cells.

In certain embodiments, the dCas9 comprises a dCas9 domain and a transcriptional activator. In some cases, the library of sgRNAs is targeted to a region between 0-750 bp upstream of the transcription start site of the METTL17 gene. In some cases, the dCas9 comprises a dCas9 domain and a transcriptional repressor. In some cases, the library of sgRNAs is targeted to a region between 0-1000 bp downstream of the transcription start site of the METTL17 gene. In some cases, wherein the dCas9 comprises: a first dCas9 fused to a transcriptional repressor; and a second dCas9 fused to a transcriptional activator; or a second dCas9 fused to an epitope fusion domain. In some cases, at least a portion of the plurality of test cells comprise a Cas9 nuclease.

### Base Editing Systems

The present disclosure also provides for base editing systems. In general, such a system may comprise a deaminase (e.g., an adenosine deaminase or cytidine deaminase) associated (e.g., fused) with a Cas5-HNH or Cas8-HNH polypeptide, e.g., Cas5-HNH or Cas8-HNH protein. The Cas5-HNH or Cas8-HNH polypeptide may be a dead Cas5-HNH or Cas8-HNH polypeptide (such as a Cas5-HNH or Cas8-HNH polypeptide nickase, e.g., engineered from a Cas5-HNH or Cas8-HNH polypeptide). In certain examples, the nucleotide deaminase is a mutated form of an adenosine deaminase The mutated form of the adenosine deaminase may have both adenosine deaminase and cytidine deaminase activities.

In example embodiments, the Cas5-HNH or Cas8-HNH is in a complex or cascade with other Cas proteins (e.g., Cas7, Cas8, etc . . . ). In general, such a complex or cascade may comprise a deaminase associated with one or more of the other Cas proteins.

In some examples, the present disclosure provides an engineered, non-naturally occurring composition comprising: the nucleic acid-guided nuclease that is catalytically inactive, a nucleotide deaminase associated with or otherwise capable of forming a complex with the Cas5-HNH or Cas8-HNH polypeptide, and a single guide or scaffold sequence capable of forming a complex with the Cas5-HNH or Cas8-HNH polypeptide and directing site-specific binding at a target sequence.

In one aspect, the present disclosure provides an engineered adenosine deaminase. The engineered adenosine deaminase may comprise one or more mutations herein. In one embodiment, the engineered adenosine deaminase has cytidine deaminase activity. In certain examples, the engineered adenosine deaminase has both cytidine deaminase activity and adenosine deaminase. In some cases, the modifications by base editors herein may be used for targeting post-translational signaling or catalysis. In one embodiment, compositions herein comprise nucleotide sequence comprising encoding sequences for one or more components of a base editing system. A base-editing system may comprise a deaminase (e.g., an adenosine deaminase or cytidine deaminase) fused with a Cas5-HNH or Cas8-HNH polypeptide or a variant thereof. In some cases, the target polynucleotide is edited at one or more bases to introduce a G→A or C→T mutation.

In some cases, the adenosine deaminase is double-stranded RNA-specific adenosine deaminase (ADAR). Examples of ADARs include those described Yiannis A Savva et al., The ADAR protein family, Genome Biol. 2012; 13 (12): 252, which is incorporated by reference in its entirety. In some examples, the ADAR may be hADAR1. In certain examples, the ADAR may be hADAR2. The sequence of hADAR2 may be that described under Accession No. AF525422.1.

In some cases, the deaminase may be a deaminase domain, e.g., a deaminase domain of ADAR (“ADAR-D”). In one example, the deaminase may be the deaminase domain of hADAR2 (“hADAR2-D), e.g., as described in Phelps K J et al., Recognition of duplex RNA by the deaminase domain of the RNA editing enzyme ADAR2. Nucleic Acids Res. 2015 January;43 (2): 1123-32, which is incorporated by reference herein in its entirety. In a particular example, the hADAR2-D has a sequence comprising amino acid 299-701 of hADAR2-D, e.g., amino acid 299-701 of the sequence under Accession No. AF525422.1.

In certain examples, the system comprises a mutated form of an adenosine deaminase fused with a dead Cas5-HNH or dead Cas8-HNH polypeptide nuclease (e.g., a Cas5-HNH or Cas8-HNH polypeptide nickase). The mutated form of the adenosine deaminase may have both adenosine deaminase and cytidine deaminase activities. In one embodiment, the adenosine deaminase may comprise one or more of the mutations: E488Q based on amino acid sequence positions of hADAR2-D, and mutations in a homologous ADAR protein corresponding to the above. In one embodiment, the adenosine deaminase may comprise one or more of the mutations: E488Q, V351G, based on amino acid sequence positions of hADAR2-D, and mutations in a homologous ADAR protein corresponding to the above. In one embodiment, the adenosine deaminase may comprise one or more of the mutations: E488Q, V351G, S486A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, V440I based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, V440I, S495N based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, V440I, S495N, K418E based on amino acid sequence positions of hADAR2-D, and mutations in a homologous ADAR protein corresponding to the above. In one embodiment, the adenosine deaminase may comprise one or more of the mutations: E488Q, V351G, S486A, T375S, S370C, P462A, N597I, L332I, 1398V, K350I, M383L, D619G, S582T, V440I, S495N, K418E, S661T based on amino acid sequence positions of hADAR2-D, and mutations in a homologous ADAR protein corresponding to the above. In some examples, provided herein includes a mutated adenosine deaminase e.g., an adenosine deaminase comprising one or more mutations of E488Q, V351G, S486A, T375S, S370C, P462A, N597I, L332I, 1398V, K350I, M383L, D619G, S582T, V440I, S495N, K418E, S661T, fused with a dead Cas5-HNH or dead Cas8-HNH polypeptide nuclease or Cas5-HNH or Cas8-HNH polypeptide nickase. In some examples, provided herein includes a mutated adenosine deaminase e.g., an adenosine deaminase comprising E488Q, V351G, S486A, T375S, S370C, P462A, N597I, L332I, 1398V, K350I, M383L, D619G, S582T, V440I, S495N, K418E, and S661T, fused with a dead Cas5-HNH or dead Cas8-HNH polypeptide nuclease or Cas5-HNH or Cas8-HNH polypeptide nickase. In some examples, provided herein includes a mutated adenosine deaminase e.g., an adenosine deaminase comprising E488Q, V351G, S486A, T375S, S370C, P462A, N597I, L332I, 1398V, K350I, M383L, D619G, S582T, V440I, S495N, K418E, S661T, and S375N fused with a dead Cas5-HNH or dead Cas8-HNH polypeptide nuclease or Cas5-HNH or Cas8-HNH polypeptide nickase.

In one embodiment, the adenosine deaminase may be a tRNA-specific adenosine deaminase or a variant thereof. In one embodiment, the adenosine deaminase may comprise one or more of the mutations: W23L, W23R, R26G, H36L, N37S, P48S, P48T, P48A, I49V, R51L, N72D, L84F, S97C, A106V, D108N, H123Y, G125A, A142N, S146C, D147Y, R152H, R152P, E155V, I156F, K157N, K161T, based on amino acid sequence positions of E. coli TadA, and mutations in a homologous deaminase protein corresponding to the above. In one embodiment, the adenosine deaminase may comprise one or more of the mutations: D108N based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, based on amino acid sequence positions of E. coli TadA, and mutations in a homologous deaminase protein corresponding to the above. In one embodiment, the adenosine deaminase may comprise one or more of the mutations: A106V, D108N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, 1156F, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, A142N, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, based on amino acid sequence positions of E. coli TadA, and mutations in a homologous deaminase protein corresponding to the above. In one embodiment, the adenosine deaminase may comprise one or more of the mutations: A106V, D108N, D147Y, E155V, L84F, H123Y, I156F, H36L, R51L, S146C, K157N, P48S, W23R, P48A, R152P, A142N, based on amino acid sequence positions of E. coli TadA, and mutations in a homologous deaminase protein corresponding to the above.

In some examples, the base editing systems may comprise an intein-mediated trans-splicing system that enables in vivo delivery of a base editor, e.g., a split-intein cytidine base editors (CBE) or adenine base editor (ABE) engineered to trans-splice. Examples of such base editing systems include those described in Colin K. W. Lim et al., Treatment of a Mouse Model of ALS by In Vivo Base Editing, Mol Ther. 2020 Jan. 14. pii: S1525-0016 (20) 30011-3. doi: 10.1016/j.ymthe.2020.01.005; and Jonathan M. Levy et al., Cytosine and adenine base editing of the brain, liver, retina, heart and skeletal muscle of mice via adeno-associated viruses, Nature Biomedical Engineering volume 4, pages 97-110 (2020), which are incorporated by reference herein in their entireties.

Examples of base editing systems include those described in International Patent Publication Nos. WO 2019/071048 (e.g. paragraphs [0933]-[0938]), WO 2019/084063 (e.g., paragraphs [0173]-[0186], [0323]-[0475], [0893]-[1094]), WO 2019/126716 (e.g., paragraphs [0290]-[0425], [1077]-[1084]), WO 2019/126709 (e.g., paragraphs [0294]-[0453]), WO 2019/126762 (e.g., paragraphs [0309]-[0438]), WO 2019/126774 (e.g., paragraphs [0511]-) , Cox DBT, et al., RNA editing with CRISPR-Cas13, Science. 2017 Nov. 24;358 (6366): 1019-1027; Abudayyeh O O, et al., A cytosine deaminase for programmable single-base RNA editing, Science 26 Jul. 2019: Vol. 365, Issue 6451, pp. 382-386; Gaudelli N M et al., Programmable base editing of A. T to G. C in genomic DNA without DNA cleavage, Nature volume 551, pages 464-471 (23 Nov. 2017); Komor A C, et al., Programmable editing of a target base in genomic DNA without double-stranded DNA cleavage. Nature. 2016 May 19;533 (7603): 420-4; Jordan L. Doman et al., Evaluation and minimization of Cas9-independent off-target DNA editing by cytosine base editors, Nat Biotechnol (2020). doi.org/10.1038/s41587-020-0414-6; and Richter M F et al., Phage-assisted evolution of an adenine base editor with improved Cas domain compatibility and activity, Nat Biotechnol (2020). doi.org/10.1038/s41587-020-0453-z, which are incorporated by reference herein in their entireties and can be used to adapt to the Cas5-HNH or Cas8-HNH polypeptide.

### Prime Editing Systems

In one embodiment, the present disclosure provides compositions and systems may comprise a Cas5-HNH or Cas8-HNH polypeptide or a catalytically inactive form, one or more guide sequences, and a reverse transcriptase. The systems may be used to insert a donor polynucleotide to a target polynucleotide. In some examples, the composition or system comprises a catalytically inactive Cas5-HNH or Cas8-HNH polypeptide, a reverse transcriptase associated with or otherwise capable of forming a complex with the Cas5-HNH or Cas8-HNH polypeptide, and a guide sequence capable of forming a complex with the Cas5-HNH or Cas8-HNH polypeptide and directing site-specific binding of the complex to a target sequence of a target polynucleotide, the guide sequence further comprising a donor sequence for insertion into the target polynucleotide.

In some cases, the catalytically inactive Cas5-HNH or Cas8-HNH polypeptide is a nickase, e.g., a DNA nickase. In some cases, the Cas5-HNH or Cas8-HNH polypeptide has one or more mutations.

The Cas5-HNH or Cas8-HNH polypeptide may be associated with a reverse transcriptase. A reverse transcriptase domain may be a reverse transcriptase or a fragment thereof. In certain aspects, the reverse transcriptase is Human immunodeficiency virus (HIV) RT, Avian myoblastosis virus (AMV) RT, Moloney murine leukemia virus (M-MLV) RT a group II intron RT, a group II intron-like RT, or a chimeric RT. In an embodiment, the RT comprises modified forms of these RTs, such as, engineered variants of Avian myoblastosis virus (AMV) RT, Moloney murine leukemia virus (M-MLV) RT, or Human immunodeficiency virus (HIV) RT (see, e.g., Anzalone, et al., Search-and-replace genome editing without double-strand breaks or donor DNA, Nature. 2019 December;576 (7785): 149-157).

In some examples, the compositions and systems may comprise the Cas5-HNH or Cas8-HNH polypeptide disclosed herein; a reverse transcriptase (RT) polypeptide connected to or otherwise capable of forming a complex with the Cas5-HNH or Cas8-HNH polypeptide; and a guide sequence capable of forming a complex with the Cas5-HNH or Cas8-HNH polypeptide and comprising: a guide sequence capable of directing site-specific binding of the Cas5-HNH or Cas8-HINH polypeptide/RNP complex to a target sequence of a target polynucleotide; a 3′ binding site region capable of binding to a cleaved upstream strand of the target polynucleotide; and a RT template sequence encoding an extended sequence, wherein the extended sequence comprises a variant region and a 3′ homologous sequence capable of hybridization to the downstream cleaved strand of the target polynucleotide.

A reverse transcriptase domain may be a reverse transcriptase or a fragment thereof. A wide variety of reverse transcriptases (RT) may be used in alternative embodiments of the present invention, including prokaryotic and eukaryotic RT, provided that the RT functions within the host to generate a donor polynucleotide sequence from the RNA template. If desired, the nucleotide sequence of a native RT may be modified, for example using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In an embodiment, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some cases, in some examples, the RT domain may be non-retron RT, e.g., a viral RT or human endogenous RTs. In some examples, the RT domain may be retron RT or DGRs RT. In some examples, the RT may be less mutagenic than a counterpart wildtype RT. In one embodiment, the RT herein is not mutagenic.

The reverse transcriptase may be fused to the C-terminus of a Cas5-HNH or Cas8-HINH polypeptide. Alternatively, or additionally, the reverse transcriptase may be fused to the N-terminus of a Cas5-HNH or Cas8-HNH polypeptide. The fusion may be via a linker and/or an adaptor protein. In some examples, the reverse transcriptase may be an M-MLV reverse transcriptase or variant thereof. The M-MLV reverse transcriptase variant may comprise one or more mutations. For the examples, the M-MLV reverse transcriptase may comprise D200N, L603W, and T330P. In another example, the M-MLV reverse transcriptase may comprise D200N, L603W, T330P, T306K, and W313F. In a particular example, the fusion of Cas5-HNH or Cas8-HNH polypeptide and reverse transcriptase is Cas5-HNH or Cas8-HNH polypeptide (with a mutation corresponding to H840A of SpCas9) fused with M-MLV reverse transcriptase (D200N+L603W+T330P+T306K+W313F).

In one embodiment, the Cas5-HNH or Cas8-HNH polypeptide herein may target DNA using a guide sequence RNA containing a binding sequence that hybridizes to the target sequence on the DNA. The guide sequence RNA may further comprise an editing sequence that contains new genetic information that replaces target DNA nucleotides. The small sizes of the Cas5-HNH or Cas8-HNH polypeptide herein may allow easier packaging and delivery of the prime editing system, e.g., with a viral vector, e.g., AAV or lentiviral vector.

A single-strand break (a nick) may be generated on the target DNA by the Cas5-HNH or Cas8-HNH polypeptide at the target site to expose a 3′-hydroxyl group, thus priming the reverse transcription of an edit-encoding extension on the guide directly into the target site. These steps may result in a branched intermediate with two redundant single-stranded DNA flaps: a 5′ flap that contains the unedited DNA sequence, and a 3′ flap that contains the edited sequence copied from the guide or scaffold sequence. The 5′ flaps may be removed by a structure-specific endonuclease, e.g., FEN122, which excises 5′ flaps generated during lagging-strand DNA synthesis and long-patch base excision repair. The non-edited DNA strand may be nicked to induce bias DNA repair to preferentially replace the non-edited strand. Examples of prime editing systems and methods include those described in Anzalone A V et al., Search-and-replace genome editing without double-strand breaks or donor DNA, Nature. 2019 Oct. 21. doi: 10.1038/s41586-019-1711-4, which is incorporated by reference herein in its entirety.

The Cas5-HNH or Cas8-HNH polypeptide (e.g., the nickase form) may be used to prime-edit a single nucleotide on a target DNA. Alternatively or additionally, the Cas5-HNH or Cas8-HNH polypeptide may be used to prime-edit at least 2, at least 3, at least 4, at least 5, at least 6, at least 7, at least 8, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 200, at least 300, at least 400, at least 500, at least 600, at least 700, at least 800, at least 900, or at least 1000 nucleotides on a target DNA.

In yet another embodiment, PRIME editing is used first to create a longer 3′ region (e.g., 20 nucleotides). Examples of prime editing systems and methods include those described in Anzalone A V et al., Search-and-replace genome editing without double-strand breaks or donor DNA, Nature. 2019 Oct. 21. doi: 10.1038/s41586-019-1711-4, which is incorporated by reference herein in its entirety. In such cases, the system comprises a Cas5-HNH or Cas8-HNH polypeptide with nickase activity, a reverse transcriptase domain, and a DNA polymerase, and a guide or scaffold molecule comprising a binding sequence capable of hybridizing to the target polynucleotide and an editing sequence. The generated region may be further extended on a DNA template as described herein. The latter may allow generation of a target-independent sequence, compatible with a generic donor sequence.

The Cas5-HNH or Cas8-HNH polypeptide is capable of generating a first cleavage in the target sequence and a second cleavage outside the target sequence on the target polynucleotide. In some variations, a second Cas5-HNH or Cas8-HNH polypeptide-mediated cleavage in vicinity to the target site may be made, which may enable more efficient invasion of the extended DNA.

In some examples, the compositions and systems of the Cas5-HNH or Cas8-HNH polypeptide herein comprise: a reverse transcriptase (RT) polypeptide connected to or otherwise capable of forming a complex with the Cas5-HNH or Cas8-HNH polypeptide; a first guide or scaffold sequence capable of forming a first Cas5-HNH or Cas8-HNH polypeptide-Reverse transcriptase complex with the Cas5-HNH or Cas8-HNH polypeptide and comprising: a guide or scaffold sequence capable of directing site-specific binding of the first Cas5-HNH or Cas8-HNH polypeptide-Reverse transcriptase complex to a first target sequence of a target polynucleotide; a first binding site region capable of binding to a cleaved or nicked strand of the target polynucleotide; and a RT template sequence encoding a first extended sequence; a second guide sequence capable of forming a second Cas5-HNH or Cas8-HNH polypeptide-Reverse transcriptase complex with the Cas5-HNH or Cas8-HNH polypeptide and comprising: a guide sequence capable of directing site specific binding of the second Cas5-HINH or Cas8-HNH polypeptide-Reverse transcriptase complex to a second target sequence of the target polynucleotide; a second binding site region capable of binding to a cleaved or nicked strand of the target polynucleotide; and a RT template sequence encoding a second extended sequence.

In some cases, the compositions and systems may further comprise: a donor template; a third guide sequence capable of forming a Cas5-HNH or Cas8-HNH polypeptide-Reverse transcriptase complex-guide sequence with the Cas5-HNH or Cas8-HNH polypeptide and comprising: a guide sequence capable of directing site-specific binding to a target sequence on the donor template; a third binding region capable of binding to a cleaved or nicked strand of the donor template; and a RT template encoding a third extended region complementary to the first extended region generated on the target polynucleotide; and a fourth guide sequence capable of forming a Cas5-HNH or Cas8-HNH polypeptide-Reverse transcriptase complex with the Cas5-HNH or Cas8-HNH polypeptide and comprising: a guide sequence capable of directing site-specific binding to a second target sequence on the donor template; a fourth binding region capable of binding to a cleaved or nicked strand of the donor template; and a RT template encoding a fourth extended region complementary to the second extended region generated on the target polynucleotide.

In some cases, the compositions and systems may further comprise a site-specific recombinase, and wherein the first and second extended regions are complementary to each other and introduce a serine integrase recombination site; and a donor molecule comprising a donor sequence for insertion into the target polypeptide and the complementary recombination site to the serine integrase recombination site.

In some examples, the compositions and systems may further comprise a recombinase. The recombinase is connected to or otherwise capable of forming a complex with the Cas5-HNH or Cas8-HNH polypeptide. In an embodiment, the complex is capable of inserting a recombination site in the DNA loci of interest by extension of RT templates that encode for the recombination site on the 3′ extension of the guide or scaffold sequences by the reverse transcriptase. In an embodiment, a donor template comprising a compatible recombination site is provided that can recombine unidirectionally with the inserted recombination site when a recombinase specific for the recombination site is also provided. In an embodiment, the donor template is a plasmid comprising the complementary recombination site and any sequence for insertion at the DNA loci of interest. In an embodiment, the recombinase is connected to or capable of forming a complex with the Cas5-HNH or Cas8-HNH polypeptide, such that all of the enzymatic proteins are brought into contact at the loci of interest. In an embodiment, the recombinase is codon optimized for eukaryotic cells (described further herein). In an embodiment, the recombinase includes a NLS (described further herein). In an embodiment, the recombinase is provided as a separate protein. The separate recombinase may form a dimer and bind to the donor template recombination site. The recombinase may be targeted to the loci of interest as a result of the insertion of the compatible recombination site that is also recognized by the recombinase. Thus, the recombinase may recognize the recombination site inserted at the DNA loci of interest and the recombination site on the donor and be targeted to the DNA loci of interest without any additional modifications to the recombinase.

In an embodiment, a second Cas5-HNH or Cas8-HNH complex connected to a recombinase is targeted to the DNA loci of interest. In an embodiment, the second Cas5-HNH or Cas8-HNH complex comprises a dead Cas5-HNH or Cas8-HNH polypeptide (dCas5-HNH or dCas8-HNH, described further herein), such that the recombinase is targeted to the DNA loci of interest, but the target sequence is not further cleaved. In an embodiment, the dCas5-HNH or dCas8-HNH targets a sequence generated only after the insertion of the recombination site. In an embodiment, the recombinase recognizes and binds to the donor template recombination site and the inserted recombination site. In an embodiment, the recombinase forms a dimer with a recombinase provided as a separate protein.

As used herein, the term “Recombinase” refers to an enzyme that catalyzes recombination between two or more recombination sites (e.g., an acceptor and donor site). Recombinases useful in the present invention catalyze recombination at specific recombination sites which are specific polynucleotide sequences that are recognized by a particular recombinase. “Uni-directional recombinases” or “integrases” refer to recombinase enzymes whose recognition sites are destroyed after the recombination has taken place. The term “integrase” refers to a type of recombinase. In other words, the sequence recognized by the recombinase is changed into one that is not recognized by the recombinase upon recombination. As a result, once a sequence is subjected to recombination by the uni-directional recombinase, the continued presence of the recombinase cannot reverse the previous recombination event.

“Recombination sites” are specific polynucleotide sequences that are recognized by the recombinase enzymes described herein. Typically, two different sites are involved (in regard to recombination termed “complementary sites”), one present in the target nucleic acid (e.g., a chromosome or episome of a eukaryote) and another on the nucleic acid that is to be integrated at the target recombination site. The terms “attB” and “attP,” which refer to attachment (or recombination) sites originally from a bacterial target (attachment site of bacteria) and a phage donor (attachment site of phage), respectively, are used herein although recombination sites for particular enzymes may have different names. The two attachment sites can share as little sequence identity as a few base pairs. The recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is again the core region. Upon recombination between the attB and attP sites, and concomitant integration of a nucleic acid at the target, the recombination sites that flank the integrated DNA are referred to as “attL” and “aatR.” The attL and attR sites, using the terminology above, thus consist of BOP′ and POB′, respectively. In some representations herein, the “O” is omitted and attB and attP, for example, are designated as BB′ and PP′, respectively.

### Recombinase/Integrase Systems

The systems and compositions herein may comprise an Cas5-HNH or Cas8-HNH polypeptide-guide sequence system, and one or more components of a recombinase or integrase. In an aspect, the Cas5-HNH or Cas8-HNH polypeptide is naturally catalytically inactive and utilized with one or more nucleic acid components to provide site-specific targeting, and the one or more components of the recombinase to introduce a modification. In an aspect, the Cas5-HNH or Cas8-HNH polypeptide may be catalytically inactivated via mutation of one or more residues of a catalytic domain or via truncation and utilized with one or more RNA components to provide site-specific targeting, and the one or more components of the recombinase introduce a modification. In an embodiment, the Cas5-HNH or Cas8-HNH polypeptide is naturally catalytically inactive. In one embodiment, a naturally inactive Cas5-HNH or Cas8-HNH is provided with a recombinase, e.g., an integrase, and optionally a reverse transcriptase.

A recombinase generally is an enzyme that mediates recombination, e.g., breaking and rejoining, of nucleic acids at specific points. DNA site-specific recombinases include serine integrases, which are phage-encoded site-specific recombinases that promote conservative recombination reactions between DNA substrates located on the phage (phage attachment site, attP) and bacterial attachment site, attB. In one embodiment, the recombinase is a serine integrase that drives a highly directions site-specific recombination.

In preferred embodiments, the recombinase mediates unidirectional site-specific recombination. In one embodiment, the recombinase is a serine recombinase (SR) also referred to as a serine integrase, encoded, for example, by IS607 family, Tn4451, and bacteriophage phiC31. See, generally, Smith M C, Thorpe H M: Diversity in the serine recombinases. Mol Microbiol. 2002, 44:299-307. 10.1046/j. 1365-2958.2002.02891.x; Li et al., (2018) J. Mol. Biol. 430:21, 4401-4418.

In an embodiment, the recombinase is a tyrosine recombinase (YR) encoded by IS91, Helitron, IS200/IS605, Crypton or DIRS-retrotransposon families. See, generally, Goodwin T J, Butler M I, Poulter T: Cryptons: a group of tyrosine-recombinase-encoding DNA transposons from pathogenic fungi. Microbiology. 2003, 149:3099-3109. Doi: 10.1099/mic.0.26529-0; Cappello J, Handelsman K, Lodish H F: Sequence of Dictyostelium DIRS-1: an apparent retrotransposon with inverted terminal repeats and an internal circle junction sequence. Cell. 1985, 43:105-115. 10.1016/0092-8674 (85) 90016-9.

In an aspect, the recombinase provides site-specific integration of a template that can be provided with the composition, e.g., a donor oligonucleotide. Without being bound by theory, the recombinase allows for integration independent of payload size and can coordinate strand exchange and re-ligation across multiple cell types, allowing integration of long stretches of polynucleotides. In an exemplary embodiment, the serine recombinase is PhiC31 and the target is DNA. In an aspect, the phiC31 allows for integration of a target site comprising an attP or pseudoattP recognition site. See, e.g., systembio.com/wp-content/uploads/phiC31_productsheet-1.pdf. In an embodiment utilizing phiC231, a donor oligonucleotide would be provided with an attB at sequence that facilitates attachment at the attP site of the target genome. Similar approaches of designing donor oligonucleotides with sequences complementary to attachment sites for a recombinase can be designed for use with the present invention. See, e.g., Li et al., (2018) J. Mol. Biol. 430:21, 4401-4418.

In preferred embodiments, the integrase mediates gene integration at diverse loci by directing insertion with an Cas5-HNH or Cas8-HNH nickase fused to both a reverse transcriptase and an integrase. In one embodiment, the integrase is a serine integrase, encoded, for example, BxbINT. See, generally, Ioannidi et al., “Drag-and-drop genome insertion without DNA cleavage with CRISPR-directed integrases”; doi: 10.1101/2021.11.01.466786m incorporated herein by reference in its entirety. In Ioannidi, Gootenberg, Abudayyeh, and colleagues show integration using a CRISPR-Cas9 nickase fused to a reverse transcriptase and serine integrase termed Programmable Addition via Site-specific Targeting Elements (PASTE) with delivery via a single dose of plasmids with functionality in non-dividing and primary cells, utilizing a guide RNA comprising an AttB landing site, termed attachment site-containing guide RNA were used to insert sequences, including diverse cargo sequences that can be inserted across different loci, varying in size up to about 36 kb. Additional uses of the PASTE system included gene tagging, gene replacement, gene delivery, and protein production and secretion, approaches that are contemplated for use with the Cas5-HNH or Cas8-HNH nickase and integrase approach. In an aspect, the guide or scaffold sequence RNA may comprise an AttB landing site. In an aspect, the recombinase provides site-specific integration of a template that can be provided with the composition, e.g., a donor oligonucleotide.

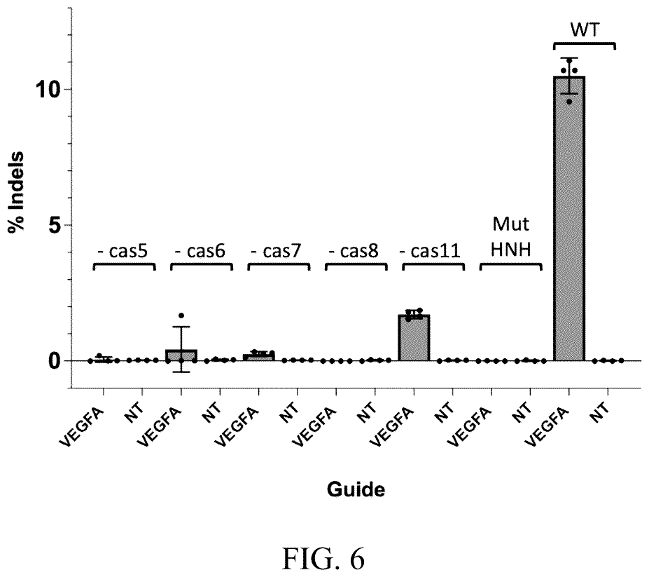

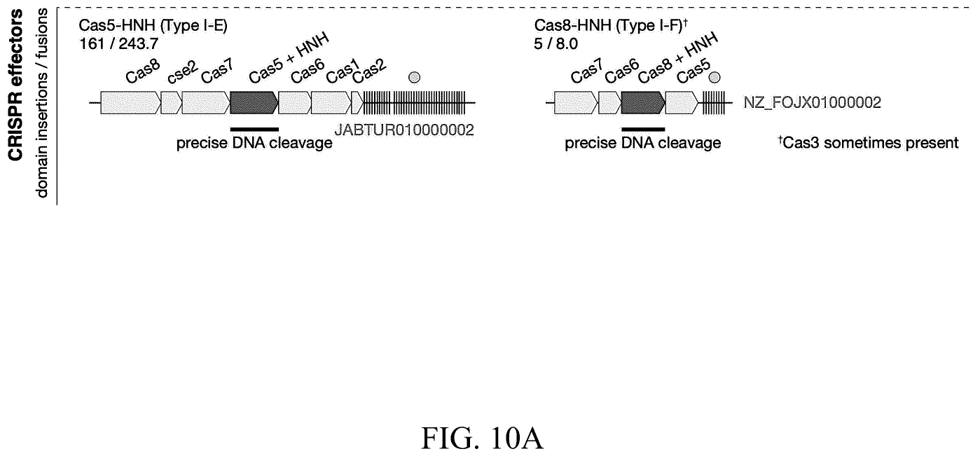

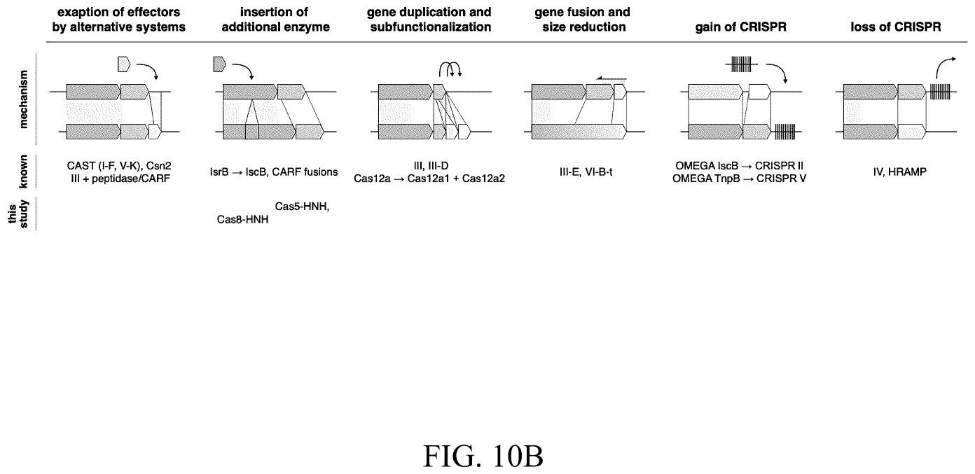

Additional large serine integrases can be used with the Cas5-HNH or Cas8-HNH nickase, for example as identified and described in Durrant et al., Large-scale discovery of recombinases for integrating DNA into the human genome, doi: 10.1101/2021.11.05.467528, incorporated herein by reference. Other integrases include BceINT, SscINT, SacINT. See, Ioannidi, 2021 at and FIG. 6d, and FIG. 10a.

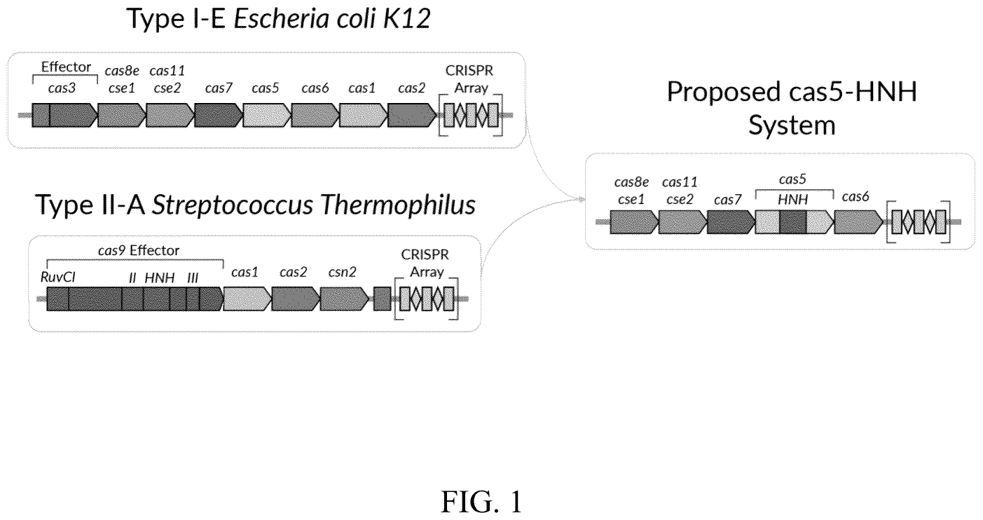

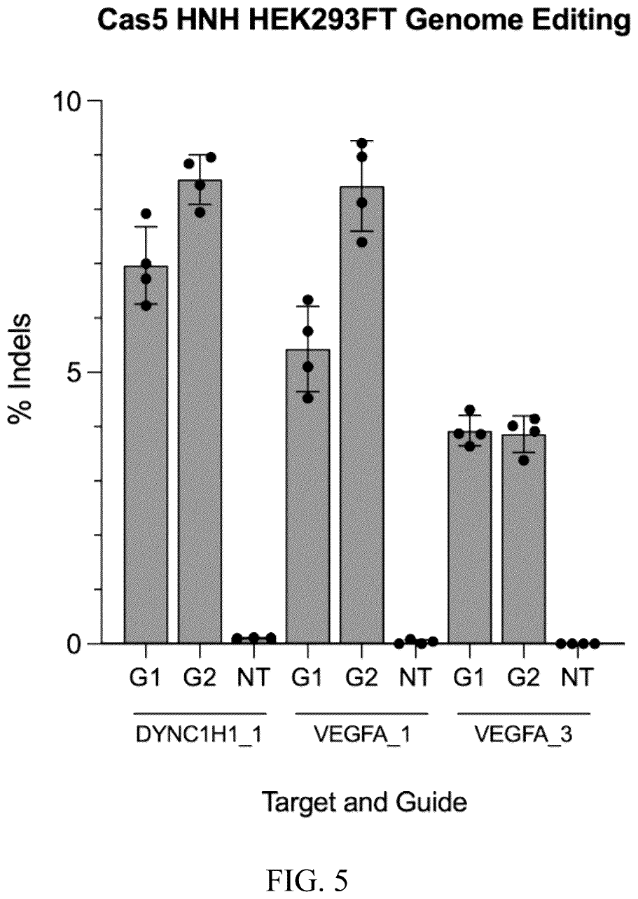

Without being bound by theory, the recombinase allows for integration independent of payload size and can coordinate strand exchange and re-ligation across multiple cell types, allowing integration of long stretches of polynucleotides. In an exemplary embodiment, the integrase is BxbINT and the target is DNA. In an aspect, the BxbINT allows for integration of a target site comprising an attP or pseudoattP recognition site. In an embodiment utilizing BxbINT, a donor oligonucleotide would be provided with an attB at sequence that facilitates attachment at the attP site of the target genome. Similar approaches of designing donor oligonucleotides with sequences complementary to attachment sites for an integrase can be designed for use with the present invention, for example a circular double-strand DNA template containing the AttP attachment site, or delivery of large cargo via an adenovirus or other viral vector, as described elsewhere herein. See, e.g., Ioannidi et al., 2021 at FIGS. 1a, 1b and 5b.

### Guided Excision-Transposition Systems

Embodiments disclosed herein provide an engineered or non-natural guided excision-transposition system. The engineered or non-natural guided excision-transposition system may comprise one or more components of a Cas5-HNH- or Cas8-HNH-guide sequence system and one or more components of a Class II transposon. The components of the Cas5-HNH- or Cas8-HNH-guide sequence system can direct the Class II transposon component(s) to retrotransposon to a target nucleic acid sequence and direct its transposition into a recipient polynucleotide.

For example, the engineered or non-natural guided excision-transposition systems that can include (a) a first Cas5-HNH or Cas8-HNH polypeptide; (b) a first Class II transposon polypeptide coupled to or otherwise capable of complexing with the first Cas5-HNH or Cas8-HNH polypeptide; (c) a first guide molecule capable of forming a first Cas5-HNH or Cas8-HNH-guide sequence complex with the first Cas5-HNH or Cas8-HNH polypeptide and directing site-specific binding to a first target sequence of a first target polynucleotide; (d) a second Cas5-HNH or Cas8-HINH polypeptide; (e) a second Class II transposon polypeptide coupled to or otherwise capable of complexing with the second Cas5-HNH or Cas8-HNH polypeptide; (f) a second guide molecule capable of forming a second Cas5-HNH or Cas8-HNH-guide sequence complex with the first Cas5-HNH or Cas8-HNH polypeptide and directing site-specific binding to a second target sequence of the first target polynucleotide; and (g) a Class II transposon polynucleotide comprising the first target polynucleotide and is capable of forming a complex with the first and second Cas5-HNH or Cas8-HNH polypeptide, the first and second guide molecules, and the first and second Class II transposon polypeptides.

In one embodiment, the engineered or non-natural guided excision-transposition system can include (h) a third guide molecule capable of complexing with the first Cas5-HNH or Cas8-HNH polypeptide and directing site-specific binding to a first target sequence of a second target polynucleotide, wherein the third guide molecule is optionally coupled to the first Cas5-HNH or Cas8-HNH polypeptide; (i) optionally, a first guide or scaffold sequence polynucleotide that encodes the third guide or scaffold sequence; (j) a fourth guide or scaffold sequence capable of complexing with the second Cas5-HNH or Cas8-HNH polypeptide and directing site-specific binding to a second target sequence of the second target polynucleotide, wherein the fourth guide molecule is optionally coupled to the second Cas5-HNH or Cas8-HNH polypeptide; and (k) optionally, a second guide molecule polynucleotide that encodes the fourth guide sequence.

In one embodiment, the first and the second Class II transposon polypeptides are capable of excising the first target polynucleotide from the Class II transposon polynucleotide. In one embodiment, the first and the second Class II transposon polypeptides are capable of transposing the first target polynucleotide in the second target polynucleotide. In one embodiment, the first target polynucleotide does not include one or more Class II transposon long terminal repeats.

The engineered or non-natural guided excision-transposition systems described herein can be based on a Class II transposon or Class II transposon system. The engineered or non-natural guided excision-transposition system may include a first target polynucleotide, also referred to as a donor polynucleotide or transposon and a second target polynucleotide, which is also referred to herein as a recipient polynucleotide. As used herein, “transposon” (also referred to as transposable element) refers to a polynucleotide sequence that is capable of moving form location in a genome to another. There are several classes of transposons. Transposons include retrotransposons (Class I transposons) and DNA transposons (Class II transposons). In some cases, retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide.

Any suitable transposon system can be used. Suitable transposon and systems thereof can include, but are not limited, to Sleeping Beauty transposon system (Tcl/mariner superfamily) (see e.g., Ivics et al. 1997. Cell. 91 (4): 501-510), piggyBac (piggyBac superfamily) (see e.g., Li et al. 2013 110 (25): E2279-E2287 and Yusa et al. 2011. PNAS. 108 (4): 1531-1536), Tol2 (superfamily hAT), Frog Prince (Tcl/mariner superfamily) (see e.g., Miskey et al. 2003 Nucleic Acid Res. 31 (23): 6873-6881) and variants thereof.

In one embodiment, the first and/or second Class II transposon polypeptide is a DD[E/D] transposon or transposon polypeptide. In one embodiment, the first and/or the second Class II transposon polynucleotide is a Tcl/mariner, PiggyBac, Frog Prince, Tn3, Tn5, hAT, CACTA, P, Mutator, PIF/Harbinger, Transib, or a Merlin/IS1016 transposon polynucleotide. In one embodiment, the first and/or second Class II transposon polypeptide is a Tcl/mariner, PiggyBac, Frog Prince, Tn3, Tn5, hAT, CACTA, P, Mutator, PIF/Harbinger, Transib, or a Merlin/IS1016 transposon polypeptide.

Suitable Class II transposon systems and components that can be utilized can also be and are not limited to those described in e.g., and without limitation, Han et al., 2013. BMC Genomics. 14:71, doi: 10.1186/1471-2164-14-71, Lopez and Garcia-Perez. 2010. Curr. Genomics. 11 (2): 115-128; Wessler. 2006. PNAS. 103 (47): 176000-17601; Gao et al., 2017. Marine Genomics. 34:67-77; Bradic et al. 2014. Mobile DNA. 5 (12) doi: 10.1186/1759-8753-5-12; Li et al., 2013. PNAS. 110 (25) E2279-E2287; Kebriaei et al. 2017. Trends in Genetics. 33 (11): 852-870); Miskey et al. 2003. Nucleic Acid res. 31 (23): 6873-6881; Nicolas et al. 2015. Microbiol Spectr. 3 (4) doi: 10.1128/microbiolspec.MDNA3-0060-2014); W. S. Reznikoff. 1993. Annu Rev. Microbiol. 47:945-963; Rubin et al. 2001. Genetics. 158 (3): 949-957; Wicker et al. 2003. Plant Physiol. 132 (1): 52-63; Majumdar and Rio. 2015. Microbiol. Spectr. 3 (2) doi: 10.1128/microbiolspec.MDNA3-0004-2014; D. Lisch. 2002. Trends in Plant Sci. 7 (11): 498-504; Sinzelle et al. 2007. PNAS. 105 (12): 4715-4720; Han et al. 2014; Genome Biol. Evol. 6 (7): 1748-1757; Grzebelus et al. 2006; Mol. Genet. Genomics. 275 (5): 450-459; Zhang et al. 2004. Genetics. 166 (2): 971-986; Chen and Li. 2008. Gene. 408 (1-2): 51-63; and C. Feschotte. 2004. Mol. Biol. Evol. 21 (9): 1769-1780.

### Topoisomerase Systems

The one or more functional domains may be one or more topoisomerase domains. In one embodiment, an engineered system for modifying a target polynucleotide comprising: an Cas5-HNH- or Cas8-HNH-guide sequence complex; a topoisomerase domain; and a nucleic acid template comprising or encoding a donor polynucleotide to be inserted to a target sequence of the target polynucleotide. In some examples, two or more of: the Cas5-HNH or Cas8-HNH polypeptide; topoisomerase domain; and nucleic acid template may form a complex. In some examples, two or more of: the Cas5-HNH or Cas8-HNH polypeptide; topoisomerase domain, may be comprised in a fusion protein.

Topoisomerases are a class of enzymes that modify the topological state of DNA via the breakage and rejoining of nucleic acid strands. In some cases, a topoisomerase may be a DNA topoisomerase, which is an enzyme that controls and alters the topologic states of DNA during transcription and catalyzes the transient breaking and rejoining of a single strand of DNA which allows the strands to pass through one another, thus altering the topology of DNA.

In one embodiment, the topoisomerase domain is capable of ligating the donor polynucleotide with the target polynucleotide. The ligation may be achieved by sticky end or blunt end ligation. In an example, the donor polynucleotide may comprise an overhang comprising a sequence complementary to a region of the target polynucleotide. Examples of ligating the donor polynucleotide with the target polynucleotide include those of TOPO cloning, e.g., those described in “The Technology Behind TOPO Cloning,” at www.thermofisher.com/us/en/home/life-science/cloning/topo/topo-resources/the-technology-behind-topo-cloning.html.

In one embodiment, the topoisomerase domain may be associated with the donor polynucleotide. For example, the topoisomerase domain is covalently linked to the donor polynucleotide.

In one embodiment, a topoisomerase domain may be provided together with, e.g., associated (e.g., fused) with a Cas5-HNH or Cas8-HNH polypeptide (e.g., a Cas5-HNH or Cas8-HNH polypeptide or a variant thereof such as a dead Cas5-HNH or dead Cas8-HNH or a Cas5-HNH or Cas8-HNH nickase). Alternatively, or additionally, the topoisomerase domain may be on a molecule different from the Cas5-HNH or Cas8-HNH polypeptide. In some cases, the topoisomerase domain may be associated with a donor polynucleotide. For example, the topoisomerase domain may be pre-loaded covalently with a donor DNA molecule. Such design may allow for efficient ligation of only a specific cargo. The topoisomerase domain may ligate the donor polynucleotide (e.g., a DNA molecule) to a target site on a target polynucleotide (e.g., a free double-stranded DNA end). In one embodiment, the donor polynucleotide may have an overhang that comprises a sequence complementary to a region of the target polynucleotide. For example, the overhang may invade into the target polynucleotide at a cut site generated by the Cas5-HNH or Cas8-HNH polypeptide.

Examples of topoisomerases include type I, including type IA and type IB topoisomerases, which cleave a single strand of a double-stranded nucleic acid molecule, and type II topoisomerases (e.g., gyrases), which cleave both strands of a double-stranded nucleic acid molecule.

Type IA and IB topoisomerases cleave one strand of a double-stranded nucleic acid molecule. In some examples, the cleavage of a double-stranded nucleic acid molecule by type IA topoisomerases generates a 5′ phosphate and a 3′ hydroxyl at the cleavage site, with the type IA topoisomerase covalently binding to the 5′ terminus of a cleaved strand. Cleavage of a double-stranded nucleic acid molecule by type IB topoisomerases may generate a 3′ phosphate and a 5′ hydroxyl at the cleavage site, with the type IB topoisomerase covalently binding to the 3′ terminus of a cleaved strand.

Examples of Type IA topoisomerases include E. coli topoisomerase I, E. coli topoisomerase III, eukaryotic topoisomerase II, archeal reverse gyrase, yeast topoisomerase III, Drosophila topoisomerase III, human topoisomerase III, Streptococcus pneumoniae topoisomerase III, and the like, including other type IA topoisomerases. A DNA-protein adduct is formed with the enzyme covalently binding to the 5′-thymidine residue, with cleavage occurring between the two thymidine residues.

Examples of Type IB topoisomerases include the nuclear type I topoisomerases present in all eukaryotic cells and those encoded by Vaccinia and other cellular poxviruses. The eukaryotic type IB topoisomerases are exemplified by those expressed in yeast, Drosophila and mammalian cells, including human cells. Viral type IB topoisomerases are exemplified by those produced by the vertebrate poxviruses (Vaccinia, Shope fibroma virus, ORF virus, fowlpox virus, and molluscum contagiosum virus), and the insect poxvirus (Amsacta moorei entomopoxvirus).

Examples of Type II topoisomerases include, bacterial gyrase, bacterial DNA topoisomerase IV, eukaryotic DNA topoisomerase II, and T-even phage encoded DNA topoisomerases. Type II topoisomerases may have both cleaving and ligating activities. Substrate double-stranded nucleic acid molecules of type II topoisomerase can be prepared such that the type II topoisomerase can form a covalent linkage to one strand at a cleavage site. For example, calf thymus type II topoisomerase can cleave a substrate ds nucleic acid molecule containing a 5′ recessed topoisomerase recognition site positioned three nucleotides from the 5′ end, resulting in dissociation of the three nucleic acid molecule 5′ to the cleavage site and covalent binding of the topoisomerase to the 5′ terminus of the ds nucleic acid molecule. Furthermore, upon contacting such a type II topoisomerase-charged ds nucleic acid molecule with a second nucleic acid molecule containing a 3′ hydroxyl group, the type II topoisomerase can ligate the sequences together, and then is released from the recombinant nucleic acid molecule.

In some examples, the topoisomerase is DNA topoisomerase I, e.g., a Vaccinia virus topoisomerase I. The topoisomerase may be pre-loaded with a donor polynucleotide. The Vaccinia virus topoisomerase may need a target comprising a 5′—OH group.

### Retrotransposon Systems

The systems and compositions herein may comprise a Cas5-HNH or Cas8-HNH polypeptide or, one or more guide or scaffold sequence RNAs, and one or more components of a retrotransposon, e.g., a non-LTR retrotransposon. The one or more components of a retrotransposon include a retrotransposon protein and retrotransposon RNA. The systems and compositions may be used to insert a donor polynucleotide to a target polynucleotide. The systems and compositions may further comprise a donor polynucleotide.

In some examples, the present disclosure provides an engineered, non-naturally occurring composition comprising: a Cas5-HNH or Cas8-HNH polypeptide, a non-LTR retrotransposon protein associated with or otherwise capable of forming a complex with the Cas5-HNH or Cas8-HNH polypeptide; a single guide sequence capable of forming a complex with the Cas5-HNH or Cas8-HNH polypeptide and directing site-specific binding to a target sequence of a target polynucleotide. The composition may further comprise a donor construct comprising a donor polynucleotide for insertion to the target polynucleotide and located between two binding elements capable of forming a complex with the non-LTR retrotransposon protein. In some cases, the Cas5-HNH or Cas8-HNH polypeptide is engineered to have nickase activity.

In some examples, the Cas5-HNH or Cas8-HNH polypeptide is fused to the N-terminus of the non-LTR retrotransposon protein. In some examples, the Cas5-HNH or Cas8-HNH polypeptide is fused to the C-terminus of the non-LTR retrotransposon protein.

The guides may direct the fusion protein to a target sequence 5′ of the targeted insertion site, and wherein the Cas5-HNH or Cas8-HNH polypeptide generates a double-strand break at the targeted insertion site. The guides may direct the fusion protein to a target sequence 3′ of the targeted insertion site, and wherein the Cas5-HNH or Cas8-HNH polypeptide generates a double-strand break at the targeted insertion site.

The donor polynucleotide may further comprise a polymerase processing element to facilitate 3′ end processing of the donor polynucleotide sequence. The polymerase may be a DNA polymerase, e.g., DNA polymerase I. In some examples, the polymerase may be an RNA polymerase.

In some examples, the donor polynucleotide further comprises a homology region to the target sequence on the 5′ end of the donor construct, the 3′ end of the donor construct, or both. In some examples, the homology region is from 1 to 50, from 5 to 30, from 8 to 25, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 base pairs in length.

Native or wild-type non-LTR retrotransposons encode the protein machinery necessary for their self-mobilization. The non-LTR retrotransposon element comprises a DNA element integrated into a host genome. This DNA element may encode one or two open reading frames (ORFs). For example, the R2 element of Bombyx mori encodes a single ORF containing reverse transcriptase (RT) activity and a restriction enzyme-like (REL) domain. L1 elements encode two ORFs, ORF1 and ORF2. ORF1 contains a leucine zipper domain involved in protein-protein interactions and a C-terminal nucleic acid binding domain. ORF2 has a N-terminal apurinic/apyrimidinic endonuclease (APE), a central RT domain, and a C-terminal cysteine histidine rich domain. An example replicative cycle of a non-LTR retrotransposon may comprise transcription of the full-length retrotransposon element to generate an mRNA active element (retrotransposon RNA). The active element mRNA is translated to generate the encoded retrotransposon proteins or polypeptides. A ribonucleoprotein complex comprising the active element and retrotransposon protein or polypeptide is formed and this RNP facilitates integration of the active element into the genome. The RNA-transposase complex nicks the genome. The 3′ end of the nicked DNA serves as a primer to allow the reverse transcription of the transposon RNA into cDNA. Fourth, the transposase proteins integrate the cDNA into the genome.

Elements of these systems may be engineered to work within the context of the invention. For example, a non-LTR retrotransposon polypeptide may be fused to a site-specific nuclease. The binding elements that allow a non-LTR retrotransposon polypeptide to bind to the native retrotransposon DNA element, may be engineered into a donor construct to facilitate entry of a donor polynucleotide sequence into a target polypeptide.

In the present invention the protein component of the non-LTR retrotransposon may be connected to or otherwise engineered to form a complex with a site-specific nuclease. The retrotransposon RNA may be engineered to encode a donor polynucleotide sequence. Thus, in certain example embodiments, the Cas5-HNH or Cas8-HNH polypeptide, via formation of a Cas5-HNH or Cas8-HNH polypeptide complex with a guide sequence, directs the retrotransposon complex (e.g., the retrotransposon polypeptide(s) and retrotransposon RNA to a target sequence in a target polynucleotide, where the retrotransposon RNP complex facilitates integration of the donor polynucleotide sequence into the target polynucleotide. Accordingly, the one or more non-LTR retrotransposon components may comprise retrotransposon polypeptides, or function domains thereof, that facilitate binding of the retrotransposon RNA, reverse transcription of the retrotransposon RNA into cDNA, and/or integration of the donor polynucleotide into the target polynucleotide, as well as retrotransposon RNA elements modified to encode the donor polynucleotide sequence.

Examples of non-LTR retrotransposons include CRE, R2, R4, L1, RTE, Tad, R1, LOA, I, Jockey, CR1. In one example, the non-LTR retrotransposon is R2. In another example, the non-LTR retrotransposon is L1. Examples of non-LTR retrotransposons may include those described in Christensen S M et al., RNA from the 5′ end of the R2 retrotransposon controls R2 protein binding to and cleavage of its DNA target site, Proc Natl Acad Sci USA. 2006 Nov. 21;103 (47): 17602-7; Eickbush T H et al, Integration, Regulation, and Long-Term Stability of R2 Retrotransposons, Microbiol Spectr. 2015 April;3 (2):MDNA3-0011-2014. doi: 10.1128/microbiolspec.MDNA3-0011-2014; Han J S, Non-long terminal repeat (non-LTR) retrotransposons: mechanisms, recent developments, and unanswered questions, Mob DNA. 2010 May 12;1 (1): 15. doi: 10.1186/1759-8753-1-15; Malik H S et al., The age and evolution of non-LTR retrotransposable elements, Mol Biol Evol. 1999 June;16 (6): 793-805, which are incorporated by reference herein in their entireties.

Examples of the non-LTR retrotransposon polypeptides also include R2 from Clonorchis sinensis, or Zonotrichia albicollis.

A non-LTR retrotransposon may comprise multiple retrotransposon polypeptides or polynucleotides encoding same. In one embodiment, the retrotransposon polypeptides may form a complex. For example, a non-LTR retrotransposon is a dimer, e.g., comprising two retrotransposon polypeptides forming a dimer. The dimer subunits may be connected or form a tandem fusion. A Cas5-HNH or Cas8-HNH polypeptide nuclease may be associate with (e.g., connected to) one or more subunits of such complex. In some examples, the non-LTR retrotransposon is a dimer of two retrotransposon polypeptides; one of the retrotransposon polypeptides comprises nuclease or nickase activity and is connected with a Cas5-HNH or Cas8-HNH polypeptide nuclease.

The retrotransposon polypeptides may comprise one or more modifications to, for example, enhance specificity or efficiency of donor polynucleotide recognition, target-primed template recognition (TPTR). The retrotransposon polypeptides may also comprise one or more truncations or excisions to remove domains or regions of wild-type protein to arrive at a minimal polypeptide that retain donor polynucleotide recognition and TPTR. In some example embodiments, the native endonuclease activity may be mutated to eliminate endonuclease activity.

In certain example embodiments, the modifications or truncations of the non-LTR retrotransposon peptide may be in a zinc finger region, a Myb region, a basic region, a reverse transcriptase domain, a cysteine-histidine rich motif, or an endonuclease domain.

A non-LTR retrotransposon may comprise polynucleotide encoding one or more retrotransposon RNA molecules. The polynucleotide may comprise one or more regulatory elements. The regulatory elements may be promoters. The regulatory elements and promoters on the polynucleotides include those described throughout this application. For example, the polynucleotide may comprise a pol2 promoter, a pol3 promoter, or a T7 promoter.

In some cases, the polynucleotide encodes a retrotransposon RNA with at least a portion of its sequence complementary to a target sequence. For example, the 3′ end of the retrotransposon RNA may be complementary to a target sequence. The RNA may be complementary to a portion of a nicked target sequence. In one embodiment, a retrotransposon RNA may comprise one or more donor polynucleotides. In certain cases, a retrotransposon RNA may encode one or more donor polynucleotides.

A retrotransposon RNA may be capable of binding to a retrotransposon polypeptide. Such retrotransposon RNA may comprise one or more elements for binding to the retrotransposon polypeptide. Examples of binding elements include hairpin structures, pseudoknots (e.g., a nucleic acid secondary structure containing at least two stem-loop structures in which half of one stem is intercalated between the two halves of another stem), stem loops, and bulges (e.g., unpaired stretches of nucleotides located within one strand of a nucleic acid duplex). In certain examples, the retrotransposon RNA comprises one or more hairpin structures. In some examples, the retrotransposon RNA comprises one or more pseudoknots. In certain examples, a retrotransposon RNA comprises a sequence encoding a donor polynucleotide and one or more binding elements for forming a complex with the retrotransposon polypeptide. The binding elements may be located on the 5′ end or the 3′ end.

In one embodiment, a retrotransposon RNA comprises a region capable of hybridizing with an overhang of a target polynucleotide at the target site. The overhang may be a stretch of single-stranded DNA. The overhang may function as a primer for reverse transcription of at least a portion of the retrotransposon RNA to a cDNA. In some cases, a region of the cDNA may be capable of hybridizing a second overhang of the target polynucleotide. The second overhang may function as a primer for the synthesis of a second strand to generate a double-stranded cDNA. The cDNA may comprise a donor polynucleotide sequence. The two overhangs may be from different strands of the target polynucleotide.

### Reverse Transcriptase Domain

The one or more functional domains may comprise alone, or in additional to functional domains, one or more reverse transcriptase domains. In one embodiment, the systems comprise an engineered system for modifying a target polynucleotide comprising: an Cas5-HNH or Cas8-HNH polypeptide or a variant thereof (e.g., dCas5-HNH or dCas8-HNH); a reverse transcriptase (RT) domain; a RNA template comprising or encoding a donor polynucleotide to be inserted to a target sequence of the target polynucleotide; and an guide or scaffold RNA molecule (i.e., a naturally single guide RNA molecule comprising a scaffold for reprogramming).

The reverse transcriptase may generate single-strand DNA based on the RNA template. The single-strand DNA may be generated by a non-retron, retron, or diversity generating retroelement (DGR). In some examples, the single-strand DNA may be generated from a self-priming RNA template. A self-priming RNA template may be used to generate a DNA without the need of a separate primer.

A reverse transcriptase domain may be a reverse transcriptase or a fragment thereof. A wide variety of reverse transcriptases (RT) may be used in alternative embodiments of the present invention, including prokaryotic and eukaryotic RT, provided that the RT functions within the host to generate a donor polynucleotide sequence from the RNA template. If desired, the nucleotide sequence of a native RT may be modified, for example using known codon optimization techniques, so that expression within the desired host is optimized. A reverse transcriptase (RT) is an enzyme used to generate complementary DNA (cDNA) from an RNA template, a process termed reverse transcription. Reverse transcriptases are used by retroviruses to replicate their genomes, by retrotransposon mobile genetic elements to proliferate within the host genome, by eukaryotic cells to extend the telomeres at the ends of their linear chromosomes, and by some non-retroviruses such as the hepatitis B virus, a member of the Hepadnaviridae, which are dsDNA-RT viruses. Retroviral RT has three sequential biochemical activities: RNA-dependent DNA polymerase activity, ribonuclease H, and DNA-dependent DNA polymerase activity. Collectively, these activities enable the enzyme to convert single-stranded RNA into double-stranded cDNA. In an embodiment, the RT domain of a reverse transcriptase is used in the present invention. The domain may include only the RNA-dependent DNA polymerase activity. In some examples, the RT domain is non-mutagenic, i.e., does not cause mutation in the donor polynucleotide (e.g., during the reverse transcriptase process). In some examples, the RT domain may be non-retron RT, e.g., a viral RT or a human endogenous RT. In some examples, the RT domain may be retron RT or DGRs RT. In some examples, the RT may be less mutagenic than a counterpart wildtype RT. In one embodiment, the RT herein is not mutagenic.

### Retrons

In an embodiment, a donor template for homologous recombination is generated by use of a self-priming RNA template for reverse transcription. A non-limiting example of a self-priming reverse transcription system is the retron system. By the term “retron” it is meant a genetic element which encodes components enabling the synthesis of branched RNA-linked single stranded DNA (msDNA) and a reverse transcriptase. Retrons which encode msDNA are known in the art, for example, but not limited to U.S. Pat. Nos. 6,017,737; 5,849,563; 5,780,269; 5,436,141; 5,405,775; 5,320,958; CA 2,075,515; all of which are herein incorporated by reference.

In an embodiment, the reverse transcriptase domain is a retron RT domain. In an embodiment, the RNA template encodes a retron RNA template that is recognized and reverse transcribed by the retron reverse transcriptase domain. Conserved across many bacterial species, retrons are highly efficient reverse transcription systems of relatively unknown function. The retron system consists of the retron RT protein, as well as the msr and msd transcripts, which function as the primer and template sequences, respectively. All components of the retron system are expressed from a single open reading frame as a single transcript including the msr-msd and encoding the retron RT protein (Lampson, et al., 2005, Retrons, msDNA, and the bacterial genome. Cytogenet Genome Res 110:491-499). The msr element ORF of a retron provides for the RNA portion of the msDNA molecule, while the msd element ORF provides for the DNA portion of the msDNA molecule. The primary transcript from the msr-msd region is thought to serve as both a template and a primer to produce the msDNA. Synthesis of msDNA is primed from an internal rG residue of the RNA transcript using its 2′—OH group. Modification of msd, or msr may also be made to permit insertion of an RNA template encoding a donor polynucleotide within the msd without altering the functioning of or the production of msDNA. The RNA template encoding a donor polynucleotide sequence may be any length but is preferably less than about 5 kb nucleotides, or also less than about 2 kb, or also less than 500 bases, provided that an msDNA product is produced.

### Diversity Generating Retroelement Systems

In an embodiment, the one or more functional domains may be a diversity generating retroelement(s) (e.g., DGR described in US20100041033A1). In one embodiment, the DGR may insert a donor polynucleotide with its homing mechanism. For example, the DGR may be associated with a catalytically inactive Cas5-HNH or Cas8-HNH polypeptide (e.g., a dead Cas5-HNH or dead Cas8-HNH), and integrate the single-strand DNA using a homing mechanism. In some examples, the DGR may be less mutagenic than a counterpart wild-type DGR. In some examples, the DGR is not error-prone. In one embodiment, the DGR herein is not mutagenic. The non-mutagenic DGR may be a mutant of a wild-type DGR. As used herein, the term “DGR” encompasses both diversity generating retroelement polynucleotides and proteins encoded by diversity generating retroelement polynucleotides. In some examples, DGR may be proteins encoded by diversity generating retroelement polynucleotides having reverse transcriptase activity. In some examples, DGR may be proteins encoded by diversity generating retroelement polynucleotides having reverse transcriptase activity and integrase activity. In some cases, the template or donor polynucleotide may be encoded by a diversity generating retroelement polynucleotide. In certain cases, the template may be a polynucleotide different from the diversity generating retroelement polynucleotide, e.g., provided as a separate construct or molecule.

In one embodiment, the DGR herein may also include a Group II intron (and any proteins and polynucleotides encoded), which are mobile ribozymes that self-splice from precursor RNAs to yield excised intron lariat RNAs, which then invade new genomic DNA sites by reverse splicing. Examples of Group II intron include those described in Lambowitz A M et al., Group II Introns: Mobile Ribozymes that Invade DNA, Cold Spring Harb Perspect Biol. 2011 August; 3 (8): a003616.

In one embodiment, the diversity-generating retroelements (DGRs) are genetic elements that can produce targeted, massive variations in the genomes that carry these elements. In one embodiment, the DGR systems rely on error-prone reverse transcriptases to produce mutagenized cDNA (containing A-to-N mutations) from a template region (TR), to replace a segment called a variable region (VR) that is similar to the TR region—this process is called mutagenic retrohoming (see, e.g., Sharifi and Ye, MyDGR: a server for identification and characterization of diversity-generating retroelements. Nucleic Acids Res. 2019 Jul. 2; 47 (W1): W289-W294). DGRs may include a unique family of retroelements that generate sequence diversity of DNA. They exist widely in bacteria, archaea, phage and plasmid, and benefit their hosts by introducing variations and accelerating the evolution of target proteins (see, e.g., Yan et al., Discovery and characterization of the evolution, variation and functions of diversity-generating retroelements using thousands of genomes and metagenomes. BMC Genomics. 2019; 20:595). The first DGR was discovered in a Bordetella phage, BPP-1. Bordetella causes the respiratory infection in humans and many other mammals, controlled by the BvgAS signal transduction system. The surface of Bordetella is highly variable owing to the dynamic gene expression in the infectious cycle. The invasion of BPP-1 to Bordetella relies on the phage tail fiber protein Mtd. With the process of mutagenic reverse transcription and cDNA integration, DGR may introduce multiple nucleotide substitutions to Mtd gene and generates different receptor-binding molecules, thus making BPP-1 the ability to invade Bordetellae with diverse cell surfaces.

The systems may be used to generate an ssDNA donor using a retron- or DGR RT, which is then integrated by homologous recombination upon target cleavage or nicking using a Cas5-HNH or Cas8-HNH polypeptide. In one embodiment, the systems may comprise DGRs and/or Group-II intron reverse transcriptases. The homing mechanism of DGRs or Group-II introns may be used in modifying a target polynucleotide. The DGRs or Group-II introns reverse transcriptase may be guided to a target polynucleotide by tethering to a dead Cas5-HNH or Cas8-HNH nuclease, TALE, or ZF protein. In another embodiment, a non-retron/DGR reverse transcriptase (e.g., a viral RT) may be used for generating cDNA off of a self-priming RNA. In one embodiment, a ssDNA may be generated by an RT, but integrate it using a dead Cas5-HNH or Cas8-HNH polypeptide, creating an accessible R-loop instead of nicking/cleaving.

### Phosphatase Systems

The systems herein may further comprise a phosphatase domain. A phosphatase is an enzyme capable of removing a phosphate group from a molecule e.g., a nucleic acid such as DNA. Examples of phosphatases include calf intestinal phosphatase, shrimp alkaline phosphatase, Antarctic phosphatase, and APEX alkaline phosphatase.

In some examples, the 5′—OH group of in the target polynucleotide may be generated by a phosphatase. A topoisomerase compatible with a 5′ phosphate target may be used to generate stable loaded intermediates. In some cases, a Cas5-HNH or Cas8-HNH polypeptide that leaves a 5′ OH after cleaving the target polynucleotide may be used. In some cases, the phosphatase domain may be associated with (e.g., fused to) the Cas5-HNH or Cas8-HNH polypeptide. The phosphatase domain may be capable of generating a —OH group at a 5′ end of the target polynucleotide. The phosphatase may be delivered separated from other components in the system, e.g., as a separate protein, on a separate vector from other components.

### Polymerase Systems

The systems herein may further comprise a polymerase domain. A polymerase refers to an enzyme that synthesizes chains of nucleic acids. The polymerase may be a DNA polymerase or an RNA polymerase.

In one embodiment, the systems comprise an engineered system for modifying a target polynucleotide comprising: an Cas5-HNH or Cas8-HNH polypeptide; a DNA polymerase domain; and a DNA template comprising a donor polynucleotide to be inserted to a target sequence of the target polynucleotide. In some examples, two or more of: the Cas5-HNH or Cas8-HNH polypeptide; DNA polymerase domain; and DNA template may form a complex. In some examples, two or more of: the Cas5-HNH or Cas8-HNH polypeptide; DNA polymerase domain; are comprised in a fusion protein. For example, the Cas5-HNH or Cas8-HNH polypeptide and DNA polymerase domain may be comprised in a fusion protein.

In one embodiment, the systems may comprise a Cas5-HNH or Cas8-HNH polypeptide (or variant thereof such as a dCas5-HNH or dCas8-HNH polypeptide or Cas5-HNH or Cas8-HNH polypeptide nickase) and a DNA polymerase (e.g., phi29, T4, T7 DNA polymerase). The systems may further comprise a single-stranded DNA or double-stranded DNA template. The DNA template may comprise i) a first sequence homologous to a target site of the Cas5-HNH or Cas8-HINH polypeptide on the target polynucleotide, and/or ii) a second sequence homologous to another region of the target polynucleotide. In one embodiment, the template may be a synthetic single-stranded or PCR-generated DNA molecule, (optionally end-protected by modified nucleotides), or a viral genome (e.g., AAV). In another embodiment, the template is generated using a reverse transcriptase. When the system is delivered into a cell, an endogenous DNA polymerase in the cell may be used. Alternatively, or additionally, an exogenous DNA polymerase may be expressed in the cell.

The DNA template may be end-protected by one or more modified nucleotides or comprises a portion of a viral genome. In some embodiment, the DNA template comprises LNA or other modifications (e.g., at the 3′ end). The presence of LNA and/or the modifications may lead to more efficient annealing with the 3′ flap generated by Cas5-HNH or Cas8-HNH polypeptide cleavage.

Examples of DNA polymerase include Taq, Tne (exo-), Tma (exo-), Pfu (exo-), Pwo (exo-), Thermoanaerobacter thermohydrosulfuricus DNA polymerase, Thermococcus litoralis DNA polymerase I, E. coli DNA polymerase I, Taq DNA polymerase I, Tth DNA polymerase I, Bacillus stearothermophilus (Bst) DNA polymerase I, E. coli DNA polymerase III, bacteriophage T5 DNA polymerase, bacteriophage M2 DNA polymerase, bacteriophage T4 DNA polymerase, bacteriophage T7 DNA polymerase, bacteriophage phi29 DNA polymerase, bacteriophage PRD1 DNA polymerase, bacteriophage phi15 DNA polymerase, bacteriophage phi21DNA polymerase, bacteriophage PZE DNA polymerase, bacteriophage PZA DNA polymerase, bacteriophage Nf DNA polymerase, bacteriophage M2Y DNA polymerase, bacteriophage B103 DNA polymerase, bacteriophage SF5 DNA polymerase, bacteriophage GA-1 DNA polymerase, bacteriophage Cp-5 DNA polymerase, bacteriophage Cp-7 DNA polymerase, bacteriophage PR4 DNA polymerase, bacteriophage PR5 DNA polymerase, bacteriophage PR 722 DNA polymerase and bacteriophage L17 DNA polymerase.

### Ligase Systems

In general, the systems comprise a Cas5-HNH or Cas8-HNH polypeptide and a ligase associated with the Cas5-HNH or Cas8-HNH polypeptide. The Cas5-HNH or Cas8-HNH polypeptide may be recruited to the target sequence by a guide sequence RNA and generate a break on the target sequence. The guide sequence RNA may further comprise a template sequence with desired mutations or other sequence elements. The template sequence may be ligated to the target sequence to introduce the mutations or other sequence elements to the nucleic acid molecule. The Cas5-HNH or Cas8-HNH polypeptide may be a nickase that generates a single-strand break on nucleic acid molecule, and the ligase may be a single-strand DNA ligase. In one embodiment, the systems comprise a pair of Cas5-HNH or Cas8-HNH polypeptide-ligases complexes, with two distinct guide sequences. Each Cas5-HNH or Cas8-HNH polypeptide-ligase complex, can target one strand of a double-stranded polynucleotide, and work together to effectively modify the sequence of the double-stranded polynucleotides.

In some examples, the Cas5-HNH or Cas8-HNH polypeptide is associated with a ligase or functional fragment thereof. The ligase may ligate a single-strand break (a nick) generated by the Cas5-HNH or Cas8-HNH polypeptide. In certain cases, the ligase may ligate a double-strand break generated by the Cas5-HNH or Cas8-HNH polypeptide. In certain examples, the Cas5-HNH or Cas8-HNH polypeptide is associated with a reverse transcriptase or functional fragment thereof.

The present invention further provides systems and methods of modifying a nucleic acid sequence using a pair of distinct Cas5-HNH or Cas8-HNH-ligase-guide sequence RNA complexes, said systems and methods comprising: (a) an engineered Cas5-HNH or Cas8-HNH polypeptide connected to or complexed with a ligase; (b) two distinct guide RNA sequences complexed with such Cas5-HNH or Cas8-HNH-ligase protein complex to form a first and a second distinct Cas5-HNH or Cas8-HNH-ligase guide or scaffold sequence complexes; (c) the first Cas5-HNH or Cas8-HNH-ligase-guide sequence RNA complex binding to one strand of a target double-stranded polynucleotide sequence, and the second Cas5-HNH or Cas8-HNH polypeptide-ligase-guide sequence RNA complex binding to another strand of the target double-stranded polynucleotide sequence; (d) upon binding of the said complexes to the locus of interest the effector protein induces the modification of the sequences associated with or at the target locus of interest, whereby the two Cas5-HNH or Cas8-HNH polypeptide-ligase-guide sequence RNA complexes work together on different strands of the double-stranded target sequence and modify the sequence.

One of the advantages of using such a “pair” of Cas5-HNH- or Cas8-HNH-ligase-guide or scaffold sequence RNA complexes includes high efficiency in modifying the sequence associated with or at the locus of interest of target double-stranded polynucleotides.

In one embodiment, the Cas5-HNH or Cas8-HNH polypeptide can be a nickase. In a preferred embodiment, a ligase is linked to the Cas5-HNH or Cas8-HNH polypeptide. The ligase can ligate the donor sequence to the target sequence. The ligase can be a single-strand DNA ligase or a double-strand DNA ligase. The ligase can be fused to the carboxyl-terminus of a Cas5-HNH or Cas8-HNH polypeptide, or to the amino-terminus of a Cas5-HNH or Cas8-HNH polypeptide.

As used herein the term “ligase” refers to an enzyme, which catalyzes the joining of breaks (e.g., double-stranded breaks or single-stranded breaks (“nicks”) between adjacent bases of nucleic acids. For example, a ligase may be an enzyme capable of forming intra- or inter-molecular covalent bonds between a 5′ phosphate group and a 3′ hydroxyl group. The term “ligate” refers to the reaction of covalently joining adjacent oligonucleotides through formation of an internucleotide linkage.

DNA ligases fall into two general categories: ATP-dependent DNA ligases (EC 6.5.1.1), and NAD (+) dependent DNA ligases (EC 6.5.1.2). NAD (+) dependent DNA ligases are found only in bacteria (and some viruses) while ATP-dependent DNA ligases are ubiquitous. The ATP-dependent DNA ligases can be divided into four classes: DNA ligase I, II, III, and IV. DNA ligase I links Okazaki fragments to form a continuous strand of DNA; DNA ligase II is an alternatively spliced form of DNA ligase III, found only in non-dividing cells; DNA ligase III is involved in base excision repair; and DNA ligase IV is involved in the repair of DNA double-strand breaks by non-homologous end joining (NHEJ). Amongst all ligases, there are two types of prokaryotic and one type of eukaryotic ligases that are particularly well suited for facilitating the blunt-ended, double-stranded DNA ligation: Prokaryotic DNA ligases (T3 and T4) and Eukaryotic DNA ligase (Ligase 1).

In some cases, the ligase is specific for double-stranded nucleic acids (e.g., dsDNA, dsRNA, RNA/DNA duplex). An example of a ligase specific for double-stranded DNA and DNA/RNA hybrids is T4 DNA ligase. In some cases, the ligase is specific for single-stranded nucleic acids (e.g., ssDNA, ssRNA). An example of such ligase is CircLigase II. In some cases, the ligase is specific for RNA/DNA duplexes. In some cases, the ligase is able to work on single-stranded, double-stranded, and/or RNA/DNA nucleic acids in any combination.

In some cases, the ligase may be a pan-ligase, which is a single ligase with the ability to ligate both DNA and RNA targets. The ligase may be specific for a target (e.g., DNA-specific or RNA-specific). In some cases, the ligase may be a dual ligase system that include DNA-specific, RNA-specific, and/or pan-ligases, in any combination.

Examples of ligases that can be used with the disclosure include T4 DNA Ligase, T3 DNA Ligase, T7 DNA Ligase, E. coli DNA Ligase, HiFi Taq DNA Ligase, 9° N™ DNA Ligase, Taq DNA Ligase, SplintR® Ligase (also known as. PBCV-1 DNA Ligase or Chlorella virus DNA Ligase), Thermostable 5′ AppDNA/RNA Ligase, T4 RNA Ligase, T4 RNA Ligase 2, T4 RNA Ligase 2 Truncated, T4 RNA Ligase 2 Truncated K227Q, T4 RNA Ligase 2, Truncated KQ, RtcB Ligase (joins single stranded RNA with a 3″-phosphate or 2′,3′-cyclic phosphate to another RNA), CircLigase II, CircLigase ssDNA Ligase, CircLigase RNA Ligase, or Ampligase® Thermostable DNA Ligas, NAD-dependent ligases including Taq DNA ligase, Thermus filiformis DNA ligase, Escherichia coli DNA ligase, Tth DNA ligase, Thermus scotoductus DNA ligase (I and II), thermostable ligase, Ampligase thermostable DNA ligase, VanC-type ligase, 9° N DNA Ligase, Tsp DNA ligase, and novel ligases discovered by bioprospecting; ATP-dependent ligases including T4 RNA ligase, T4 DNA ligase, T3 DNA ligase, T7 DNA ligase, Pfu DNA ligase, DNA ligase I, DNA ligase III, DNA ligase IV, and novel ligases discovered by bioprospecting, and wild-type, mutant isoforms, and genetically engineered variants thereof.

In one embodiment, the examples of the ligases include those used in sequencing by synthesis or sequencing by ligation reactions.

### Helitron Systems

The systems and compositions herein may comprise an Cas5-HNH or Cas8-HNH polypeptide, one or more guide or scaffold sequence RNAs, and one or more components of a helitron. The systems and compositions may be used to insert a donor polynucleotide to a target polynucleotide. The systems and compositions may further comprise a donor polynucleotide.

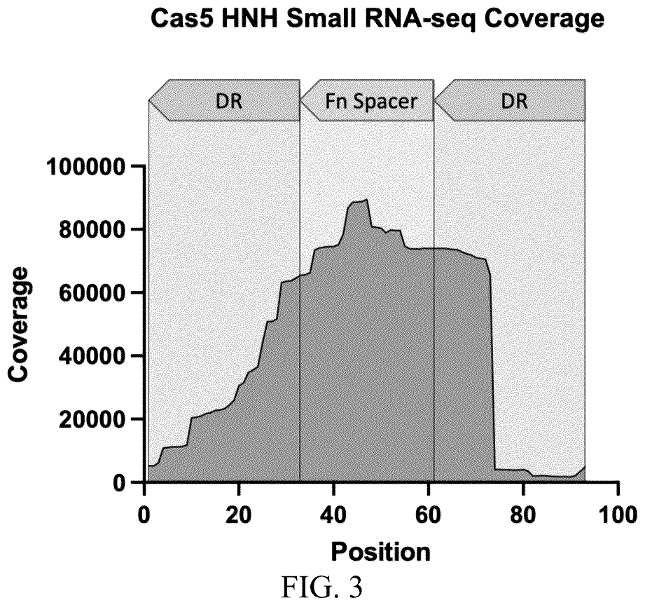

The term “helitron”, as used herein, refers to a polynucleotide (or nucleic acid segment), recognized as a transposon that captures and mobilizes gene fragments in eukaryotes. The term “helitron” as used herein refers to transposase that comprises an endonuclease domain and a C-terminal helicase domain. Helitrons are rolling-circle RNA transposons. In one embodiment, the helitron encodes a 1400 to about 2000 amino acid, or about 1800 amino acid multidomain transposase. In embodiments, the helitron comprises a hairpin near the 3′end to function as a transposition terminator. In embodiments, the transposon comprises a RepHel motif comprising a replication initiator (Rep) and a DNA helicase (hel) domain. See, Thomas J. & Pritham E. J. Helitrons, the eukaryotic rolling-circle transposable elements. Microbiol. Spectr. 3, 893-926 (2015). In embodiments, the helitron comprises a Rep nuclease domain and C-terminal helicase domain and inserts between an AT dinucleotide in single strand DNA. In an aspect, the C-terminal helicase unwinds the DNA in a 5′ to 3′ direction. The HUH nuclease domain may comprise one or two active site tyrosine residues, in embodiments, is a 2 Tyrosine (Y2) HUH endonuclease domain. Helitrons can encompass helentron, proto-helentron and helitron2 type proteins, structures of which can be as described in Thomas et al., 2015 at FIGS. 1 and 3, incorporated specifically by reference. Particular organisms in which the helitron or helentrons have been found can include those in Table 1 of Thomas J. & Pritham E. J. Helitrons, the eukaryotic rolling-circle transposable elements. Microbiol. Spectr. 3, 893-926 (2015), incorporated herein by reference. Similarly, helitrons can be identified based at least in part on the Rep motif, and conserved residues in the helitrons. Thomas J. & Pritham E. J. Helitrons, the eukaryotic rolling-circle transposable elements. Microbiol. Spectr. 3, 893-926 (2015), specifically incorporated herein by reference.

The expression “helitron reaction” used herein refers to a reaction wherein a transposase inserts a donor polynucleotide sequence in or adjacent to an insertion site on a target polynucleotide. The insertion site may contain a sequence or secondary structure recognized by the helitron and/or an insertion motif sequence in the target polynucleotide into which the donor polynucleotide sequence may be inserted.

As described in Grabundzija 2018, the helitron terminal sequences contains a distinct ˜150 base pairs (bp) long sequence with an absolutely conserved dinucleotide at the end of left terminal sequence (LTS), and a tetranucleotide at the end of right terminal sequence (RTS) which is preceded by a palindromic sequence that can form a hairpin structure. Grabundzija et al., Nat. Commun. 2018; 9:1278; doi: 10.1035/s41467-018-03688-w.

The helitron end sequences may be responsible for identifying the donor polynucleotide for transposition. The helitron end sequences may be the DNA sequences used to perform a transposition reaction, the end sequences may be referred to herein as right terminal sequences and left terminal sequence. The donor polynucleotide can be configured to comprise a first and second helitron recognition sequence that are at least 80%, 85%, 90%, 95% 96%, 97%, 98%, 99% or 100% complementary to a left terminal sequence and/or a right terminal sequence of a polynucleotide encoding the helitron polypeptide.

In an aspect, the palindromic sequence may be located upstream of the right terminal sequence, for example, about 5, 10, 15, 20, 25, 30, 35 nucleotides upstream of the right terminal sequence end, or about 10 to 15 nucleotides upstream of the right terminal sequence end, about 10 to 12 nucleotides or about 11 nucleotides upstream of the right terminal sequence end. Ivana Grabundzija, Nat Commun. 2016; 7:10716, doi: 10.1038/ncomms10716, incorporated herein by reference.

Exemplary helitrons can be identified using software, for example (EAHelitron) that has been used to identify Helitrons in a wide range of plant genomes. See, Hu, K., Xu, K., Wen, J. et al. Helitron distribution in Brassicaceae and whole Genome Helitron density as a character for distinguishing plant species. BMC Bioinformatics 20, 354 (2019). doi: 10.1186/s12859-019-2945-8, incorporated herein by reference.

The helitron may be derived from a eukaryote. In an aspect, the helitron is derived from a mammalian genome, in an aspect, vespertilionid bats, e.g., Helibat. In embodiments, the helitron is derived from derived from a Helibat1 transposon. In embodiments, the helitron is Helraiser, the full DNA sequence of the consensus transposon, including left terminal and right terminal sequences as well as hairpin identified is provided in Grabundzija, 2016 at Supplementary FIG. 1, specifically incorporated herein by reference. In an aspect, the helitron is flanked by left and right terminal sequences of the transposon. In an aspect, the left terminal sequence and right terminal sequence terminates with the conserved 5′-TC/CTAG-3′ motif. In an embodiment, the helitron may comprise a palindromic sequence that is about 10 to about 35, or about 5-25 bp or about 19-bp-long palindromic sequence with the potential to form a hairpin structure.

Elements of these systems may be engineered to work within the context of the invention. For example, a helitron polypeptide may be fused to a polypeptide capable of generating an R-loop. Fusion may be by any appropriate linker, in an exemplary embodiment, XTEN16. The binding elements that allow a helitron polypeptide to bind, for example, the use of sequences complementary to the right terminal sequence and the left terminal sequence of the helitron may be engineered into a donor construct to facilitate entry of a donor polynucleotide sequence into a target polynucleotide.

In certain example embodiments, the Cas5-HNH or Cas8-HNH polypeptide, via formation of complex with a guide or scaffold sequence, directs the helitron polypeptide to a target sequence in a target polynucleotide, where the helitron facilitates integration of a donor polynucleotide sequence into the target polynucleotide.

The helitron polypeptides may also comprise one or more truncations or excisions to remove domains or regions of wild-type protein to arrive at a minimal polypeptide, alter functionality according to the system in which the helitron is used, or mutated to enhance or diminish particular activities associated with the helitron, i.e., nuclease activity or helicase activity.

### Multiplexing

In one embodiment, Cas5-HNH or Cas8-HNH polypeptide nucleases may be used in a multiplex (tandem) targeting approach. For example, Cas5-HNH or Cas8-HNH polypeptide nuclease herein can employ more than one RNA guide without losing activity. This may enable the use of the Cas5-HNH or Cas8-HNH polypeptide, systems or complexes as defined herein for targeting multiple DNA targets, genes or gene loci, with a single enzyme, system or complex as defined herein. The guide sequence RNAs may be tandemly arranged, optionally separated by a nucleotide sequence such as a conserved nucleotide sequence as defined herein. The position of the different guide sequence RNAs is the tandem does not influence the activity.

In one aspect, the Cas5-HNH or Cas8-HNH polypeptide nucleases may be used for tandem or multiplex targeting. It is to be understood that any of the Cas5-HNH or Cas8-HNH polypeptide, complexes, or compositions herein elsewhere may be used in such an approach. Any of the methods, products, compositions and uses as described herein elsewhere are equally applicable with the multiplex or tandem targeting approach further detailed below. By means of further guidance, the following particular aspects and embodiments are provided.

In one aspect, the invention provides for the use of a Cas5-HNH or Cas8-HNH polypeptide, complex or system as defined herein for targeting multiple gene loci. In one embodiment, this can be established by using multiple (tandem or multiplex) guide or scaffold sequence RNAs. In an embodiment, a double nickase system is provided, wherein two or more Cas5-HNH or Cas8-HNH nickases are provided for modifying multiple target polynucleotides. In an aspect, the guide or scaffold sequences specifically targets its corresponding nucleic acid molecule, e.g., DNA molecule. In one embodiment, the guide or scaffold sequences target locations on opposite strands of the same double stranded DNA molecule. In an embodiment, the guide or scaffold sequences target locations on the same strand DNA molecule. In an embodiment, the two or more guide or scaffold sequences directs sequence-specific binding of the Cas5-HNH or Cas8-HNH system to sense and antisense strands of the target sequence and introduce one or more double strand break(s) to the target sequence.

In one aspect, the invention provides methods for using one or more elements of a Cas5-HNH or Cas8-HNH polypeptide, complex or system as defined herein for tandem or multiplex targeting, wherein said system herein comprises multiple guide RNA sequences. Said guide or scaffold RNA sequences are separated by a nucleotide sequence, such as a conserved nucleotide sequence as defined herein elsewhere.

The Cas5-HNH or Cas8-HNH polypeptide, compositions, systems or complexes as defined herein provides an effective means for modifying multiple target polynucleotides. The Cas5-HNH or Cas8-HNH polypeptide, system or complex as defined herein has a wide variety of utility including modifying (e.g., deleting, inserting, translocating, inactivating, activating) one or more target polynucleotides in a multiplicity of cell types. As such the Cas5-HNH or Cas8-HNH polypeptide, system or complex as defined herein of the invention has a broad spectrum of applications in, e.g., gene therapy, drug screening, disease diagnosis, and prognosis, including targeting multiple gene loci within a single system.

In one aspect, the present disclosure provides a Cas5-HNH or Cas8-HNH polypeptide, system or complex as defined herein, having a Cas5-HNH or Cas8-HNH polypeptide having at least one destabilization domain associated therewith, and multiple guide or scaffold RNAs that target multiple nucleic acid molecules such as DNA molecules, whereby each of said multiple guide or scaffold RNAs specifically targets its corresponding nucleic acid molecule, e.g., DNA molecule. In one embodiment, the Cas5-HNH or Cas8-HNH polypeptide used for multiplex targeting is associated with one or more functional domains. In some more specific embodiments, the Cas5-HNH or Cas8-HNH polypeptide used for multiplex targeting is a dead Cas5-HNH or Cas8-HNH polypeptide nuclease. The inventors have found that the Cas5-HNH or Cas8-HNH polypeptide as described herein may enable improved and/or direct access to one or more nucleotides involved in the DNA: RNA duplex.

Each nucleic acid molecule target, e.g., DNA molecule can encode a gene product or encompass a gene locus. Using multiple guide RNAs hence enables the targeting of multiple gene loci or multiple genes. In one embodiment the Cas5-HNH or Cas8-HNH polypeptide may cleave the DNA molecule encoding the gene product. In one embodiment expression of the gene product is altered. The Cas5-HNH or Cas8-HNH polypeptide and the guide RNAs do not naturally occur together. The present disclosure comprehends the guide RNAs comprising tandemly arranged guide sequences. The present disclosure further comprehends coding sequences for the Cas5-HNH or Cas8-HNH polypeptide being codon optimized for expression in a eukaryotic cell. In an embodiment the eukaryotic cell is a mammalian cell, a plant cell or a yeast cell and in a more preferred embodiment the mammalian cell is a human cell. Expression of the gene product may be decreased. The Cas5-HNH or Cas8-HNH polypeptide may form part of a system or complex, which further comprises tandemly arranged guide RNAs (gRNAs) comprising a series of 2, 3, 4, 5, 6, 7, 8, 9, 10, 15, 25, 25, 30, or more than 30 guide sequences, each capable of specifically hybridizing to a target sequence in a genomic locus of interest in a cell. In one embodiment, the functional system or complex binds to the multiple target sequences. In one embodiment, the functional system or complex may edit the multiple target sequences, e.g., the target sequences may comprise a genomic locus, and in one embodiment, there may be an alteration of gene expression. In one embodiment, the functional system or complex may comprise further functional domains. In one embodiment, the invention provides a method for altering or modifying expression of multiple gene products. The method may comprise introducing into a cell containing said target nucleic acids, e.g., DNA molecules, or containing and expressing target nucleic acid, e.g., DNA molecules; for instance, the target nucleic acids may encode gene products or provide for expression of gene products (e.g., regulatory sequences).

### Inducible Systems

In one embodiment, a Cas5-HNH or Cas8-HNH polypeptide nuclease may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc.), or light inducible systems (Phytochrome, LOV domains, or cryptochrome). In one embodiment, the Cas5-HNH or Cas8-HNH polypeptide may be a part of a Light Inducible Transcriptional Effector (LITE) to direct changes in transcriptional activity in a sequence-specific manner. The components of a light may include a Cas5-HNH or Cas8-HNH polypeptide, a light-responsive cytochrome heterodimer (e.g., from Arabidopsis thaliana), and a transcriptional activation/repression domain. Further examples of inducible DNA binding proteins and methods for their use are provided in U.S. Provisional Application Nos. 61/736,465 and U.S. 61/721,283, and International Patent Publication No. WO 2014/018423 A2 which is hereby incorporated by reference in its entirety.

### Self-Inactivating Systems

Once all copies of a gene in the genome of a cell have been edited, continued expression of the system in that cell is no longer necessary. Indeed, sustained expression would be undesirable in case of off-target effects at unintended genomic sites, etc. Thus time-limited expression would be useful. Inducible expression offers one approach, but in addition Applicants have engineered a self-inactivating system that relies on the use of a non-coding guide target sequence within the vector itself. Thus, after expression begins, the system will lead to its own destruction, but before destruction is complete it will have time to edit the genomic copies of the target gene (which, with a normal point mutation in a diploid cell, requires at most two edits). Simply, the self-inactivating system includes additional RNA (e.g., guide RNA) that targets the coding sequence for the Cas5-HNH or Cas8-HNH polypeptide itself or that targets one or more non-coding guide target sequences complementary to unique sequences present in one or more of the following: (a) within the promoter driving expression of the non-coding RNA elements, (b) within the promoter driving expression of the Cas5-HNH or Cas8-HNH polypeptide gene, (c) within 100 bp of the ATG translational start codon in the Cas5-HNH or Cas8-HNH polypeptide coding sequence, (d) within the inverted terminal repeat (iTR) of a viral delivery vector, e.g., in the AAV genome.

In some aspects, a single guide or scaffold sequence RNA is provided that is capable of hybridization to a sequence downstream of a Cas5-HNH or Cas8-HNH polypeptide start codon, whereby after a period of time there is a loss of the Cas5-HNH or Cas8-HNH polypeptide nuclease expression. In some aspects, one or more guide or scaffold sequence RNA(s) are provided that are capable of hybridization to one or more coding or non-coding regions of the polynucleotide encoding the system, whereby after a period of time there is a inactivation of one or more, or in some cases all, of the system. In some aspects of the system, and not to be limited by theory, the cell may comprise a plurality of complexes, wherein a first subset of complexes comprise a first guide or scaffold sequence RNA capable of targeting a genomic locus or loci to be edited, and a second subset of complexes comprise at least one second guide RNA capable of targeting the polynucleotide encoding the system, wherein the first subset of complexes mediate editing of the targeted genomic locus or loci and the second subset of complexes eventually inactivate the system, thereby inactivating further expression in the cell.

The various coding sequences (Cas5-HNH or Cas8-HNH polypeptide and guide RNAs) can be included on a single vector or on multiple vectors. For instance, it is possible to encode the enzyme on one vector and the various RNA sequences on another vector, or to encode the enzyme and one guide RNA on one vector, and the remaining guide sequence RNA on another vector, or any other permutation. In general, a system using a total of one or two different vectors is preferred.

Where multiple vectors are used, it is possible to deliver them in unequal numbers, and ideally with an excess of a vector which encodes the first guide or scaffold sequence RNA relative to the second guide sequence RNA, thereby assisting in delaying final inactivation of the system until genome editing has had a chance to occur.

The first guide sequence RNA can target any target sequence of interest within a genome, as described elsewhere herein. The second guide sequence RNA targets a sequence within the vector which encodes the Cas5-HNH or Cas8-HNH polypeptide, and thereby inactivates the enzyme's expression from that vector. Thus, the target sequence in the vector must be capable of inactivating expression. Suitable target sequences can be, for instance, near to or within the translational start codon for the Cas5-HNH or Cas8-HNH polypeptide coding sequence, in a non-coding sequence in the promoter driving expression of the non-coding RNA elements, within the promoter driving expression of the Cas5-HNH or Cas8-HNH polypeptide gene, within 100 bp of the ATG translational start codon in the Cas5-HNH or Cas8-HNH polypeptide nuclease coding sequence, and/or within the inverted terminal repeat (iTR) of a viral delivery vector, e.g., in the AAV genome. A double stranded break near this region can induce a frame shift in the Cas5-HNH or Cas8-HNH polypeptide nuclease coding sequence, causing a loss of protein expression. An alternative target sequence for the “self-inactivating” guide RNA would aim to edit/inactivate regulatory regions/sequences needed for the expression of the system or for the stability of the vector. For instance, if the promoter for the Cas5-HNH or Cas8-HNH polypeptide coding sequence is disrupted then transcription can be inhibited or prevented. Similarly, if a vector includes sequences for replication, maintenance, or stability then it is possible to target these. For instance, in a AAV vector a useful target sequence is within the iTR. Other useful sequences to target can be promoter sequences, polyadenylation sites, etc.

Furthermore, if the guide or scaffold sequence RNAs are expressed in array format, the “self-inactivating” guide or scaffold RNAs that target both promoters simultaneously will result in the excision of the intervening nucleotides from within the Cas5-HNH or Cas8-HNH polypeptide expression construct, effectively leading to its complete inactivation. Similarly, excision of the intervening nucleotides will result where the guide or scaffold sequence RNAs target both ITRs, or targets two or more other components simultaneously. Self-inactivation as explained herein is applicable, in general, with systems in order to provide regulation of the systems. For example, self-inactivation as explained herein may be applied to the repair of mutations, for example expansion disorders, as explained herein. As a result of this self-inactivation, repair may be only transiently active.

Addition of non-targeting nucleotides to the 5′ end (e.g., 1-10 nucleotides, preferably 1-5 nucleotides) of the “self-inactivating” guide or scaffold sequence RNA can be used to delay its processing and/or modify its efficiency as a means of ensuring editing at the targeted genomic locus prior to shut down.

In one aspect of the self-inactivating AAV system, plasmids that co-express one or more guide or scaffold sequence RNA targeting genomic sequences of interest (e.g. 1-2, 1-5, 1-10, 1-15, 1-20, 1-30) may be established with “self-inactivating” guide sequence RNAs that target an Cas5-HNH or Cas8-HNH polypeptide sequence at or near the engineered ATG start site (e.g. within 5 nucleotides, within 15 nucleotides, within 30 nucleotides, within 50 nucleotides, within 100 nucleotides). A regulatory sequence in the U6 promoter region can also be targeted with a guide or scaffold sequence RNA. The U6-driven guide RNAs may be designed in an array format such that multiple guide or scaffold sequence RNA sequences can be simultaneously released. When first delivered into target tissue/cells (left cell) guide RNAs begin to accumulate while Cas5-HNH or Cas8-HNH polypeptide levels rise in the nucleus. Cas5-HNH or Cas8-HNH polypeptide nuclease complexes with all of the guide RNAs to mediate genome editing and self-inactivation of the Cas5-HNH or Cas8-HNH polypeptide plasmids.

One aspect of a self-inactivating system is expression of singly or in tandem array format from 1 up to 4 or more different guide or scaffold sequences; e.g., up to about 20 or about 30 guide or scaffold sequences. Each individual self-inactivating guide or scaffold sequence may target a different target. Such may be processed from, e.g., one chimeric pol3 transcript. Pol3 promoters such as U6 or H1 promoters may be used. Pol2 promoters such as those mentioned throughout herein. Inverted terminal repeat (iTR) sequences may flank the Pol3 promoter-guide RNA(s)-Pol2 promoter-Cas5-HNH or Cas8-HNH polypeptide.

One aspect of a tandem array transcript is that one or more guide(s) sequences edit the one or more target(s) while one or more self-inactivating guides sequences inactivate the system. Thus, for example, the described system for repairing expansion disorders may be directly combined with the self-inactivating system described herein. Such a system may, for example, have two guides directed to the target region for repair as well as at least a third guide directed to self-inactivation of the Cas5-HNH or Cas8-HNH polypeptide or systems.

The guide sequence RNA may be a control guide. For example, it may be engineered to target a nucleic acid sequence encoding the Cas5-HNH or Cas8-HNH polypeptide itself, as described in U.S. Patent Publication No. US2015232881A1, the disclosure of which is hereby incorporated by reference. In one embodiment, a system or composition may be provided with just the guide or scaffold sequence RNA engineered to target the nucleic acid sequence encoding the Cas5-HNH or Cas8-HNH polypeptide. In addition, the system or composition may be provided with the guide or scaffold sequence RNA engineered to target the nucleic acid sequence encoding the Cas5-HNH or Cas8-HNH polypeptide, as well as nucleic acid sequence encoding the Cas5-HNH or Cas8-HNH polypeptide and, optionally a second guide RNA and, further optionally, a repair template. The second guide sequence RNA may be the primary target of the system or composition (such a therapeutic, diagnostic, knock out etc. as defined herein). In this way, the system or composition is self-inactivating. This is exemplified in relation to Cas in US2015232881A1 (also published as WO2015070083 (A1) referenced elsewhere herein, and may be extrapolated to other Cas5-HNH or Cas8-HNH polypeptide, e.g., orthologous Cas5-HNH or Cas8-HNH polypeptide.

### Histone Modifying Systems

In an example embodiment, the functional domain comprises a histone modifying domain. Functional domains may be used to regulate transcription, e.g., transcriptional repression. Transcriptional repression is often mediated by chromatin modifying enzymes such as histone methyltransferases (HMTs) and deacetylases (HDACs). Histone acetyltransferases are preferred in example embodiments. Repressive histone effector domains are known and an exemplary list is provided below. In the exemplary table, preference was given to proteins and functional truncations of small size to facilitate efficient viral packaging (for instance via AAV). In general, however, the domains may include HDACs, histone methyltransferases (HMTs), and histone acetyltransferase (HAT) inhibitors, as well as HDAC and HMT recruiting proteins. The functional domain may be or include, in example embodiments, HDAC Effector Domains, HDAC Recruiter Effector Domains, Histone Methyltransferase (HMT) Effector Domains, Histone Methyltransferase (HMT) Recruiter Effector Domains, or Histone Acetyltransferase Inhibitor Effector Domains.

HDAC Effector Domains

| | | | | | Full | | | |
|---|---|---|---|---|---|---|---|---|
| Subtype/ | | Substrate (if | Modification (if | | size | Selected | Final size | Catalytic |
| Complex | Name | known) | known) | Organism | (aa) | truncation (aa) | (aa) | domain |
| HDAC I | HDAC8 | — | — | X. laevis | 325 | 1-325 | 325 | 1-272: |
| | | | | | | | | HDAC |
| HDAC I | RPD3 | — | — | S. cerevisiae | 433 | 19-340 | 322 | 19-331: |
| | | | | | | | (Vannier) | HDAC |
| HDAC IV | MesoLo4 | — | — | M. loti | 300 | 1-300 (Gregoretti) | 300 | — |
| HDAC IV | HDAC11 | — | — | H. sapiens | 347 | 1-347 (Gao) | 347 | 14-326: |
| | | | | | | | | HDAC |
| HD2 | HDT1 | — | — | A. thaliana | 245 | 1-211 (Wu) | 211 | — |
| SIRT I | SIRT3 | H3K9Ac | — | H. sapiens | 399 | 143-399 (Scher) | 257 | 126-382: |
| | | H4K16Ac | | | | | | SIRT |
| | | H3K56Ac | | | | | | |
| SIRT I | HST2 | — | — | C. albicans | 331 | 1-331 (Hnisz) | 331 | — |
| SIRT I | CobB | — | — | E. coli (K12) | 242 | 1-242 (Landry) | 242 | — |
| SIRT I | HST2 | — | — | S. cerevisiae | 357 | 8-298 (Wilson) | 291 | — |
| SIRT III | SIRT5 | H4K8Ac | — | H. sapiens | 310 | 37-310 (Gertz) | 274 | 41-309: |
| | | H4K16Ac | | | | | | SIRT |
| SIRT III | Sir2A | — | — | P. falciparum | 273 | 1-273 (Zhu) | 273 | 19-273: |
| | | | | | | | | SIRT |
| SIRT IV | SIRT6 | H3K9Ac | — | H. sapiens | 355 | 1-289 (Tennen) | 289 | 35-274: |
| | | H3K56Ac | | | | | | SIRT |

Accordingly, the repressor domains of the present invention may be selected from histone methyltransferases (HMTs), histone deacetylases (HDACs), histone acetyltransferase (HAT) inhibitors, as well as HDAC and HMT recruiting proteins.

The HDAC domain may be any of those in the table above, namely: HDAC8, RPD3, MesoLo4, HDAC11, HDT1, SIRT3, HST2, CobB, HST2, SIRT5, Sir2A, or SIRT6.

In some embodiment, the functional domain may be a HDAC Recruiter Effector Domain. Preferred examples include those in the table below, namely MeCP2, MBD2b, Sin3a, NcoR, SALL1, RCOR1. NcoR is exemplified in the present Examples and, although preferred, it is envisaged that others in the class will also be useful.

HDAC Recruiter Effector Domains

| | | | | | Full | | Final | |
|---|---|---|---|---|---|---|---|---|
| Subtype/ | | Substrate | Modification | | size | Selected | size | |
| Complex | Name | (if known) | (if known) | Organism | (aa) | truncation (aa) | (aa) | Catalytic domain |
| Sin3a | MeCP2 | — | — | R. norvegicus | 492 | 207-492 (Nan) | 286 | — |
| Sin3a | MBD2b | — | — | H. sapiens | 262 | 45-262 (Boeke) | 218 | — |
| Sin3a | Sin3a | — | — | H. sapiens | 1273 | 524-851(Laherty) | 328 | 627-829: HDAC1 |
| | | | | | | | | interaction |
| NcoR | NcoR | — | — | H. sapiens | 2440 | 420-488 hang) | 69 | — |
| NuRD | SALL1 | — | — | M. musculus | 1322 | 1-93 (Lauberth) | 93 | — |
| CoREST | RCOR1 | — | — | H. sapiens | 482 | 81-300 (Gu, | 220 | — |
| | | | | | | Ouyang) | | |

In some embodiment, the functional domain may be a Methyltransferase (HMT) Effector Domain. Preferred examples include those in the Table below, namely NUE, vSET, EHMT2/G9A, SUV39H1, dim-5, KYP, SUVR4, SET4, SET1, SETD8, and TgSET8. NUE is exemplified in the present Examples and, although preferred, it is envisaged that others in the class will also be useful.

Histone Methyltransferase (HMT) Effector Domains

| Subtype/ | | Substrate | Modification | | Full size | Selected | Final | |
|---|---|---|---|---|---|---|---|---|
| Complex | Name | (if known) | (if known) | Organism | (aa) | truncation (aa) | size (aa) | Catalytic domain |
| SET | NUE | H2B, H3, | — | C. trachomatis | 219 | 1-219 (Pennini) | 219 | — |
| | | H4 | | | | | | |
| SET | vSET | — | H3K27me3 | P. bursaria | 119 | 1-119 (Mujtaba) | 119 | 4-112: SET2 |
| | | | | chlorella virus | | | | |
| SUV39 | EHMT2/ | H1.4K2, | H3K9me1/2, | M. musculus | 1263 | 969-1263 (Tachibana) | 295 | 1025-1233: |
| family | G9A | H3K9, | H1K25me1 | | | | | preSET, SET, |
| | | H3K27 | | | | | | postSET |
| SUV39 | SUV39 | — | H3K9me2/3 | H. sapiens | 412 | 79-412 (Snowden) | 334 | 172-412: preSET, |
| | H1 | | | | | | | SET, postSET |
| Suvar3-9 | dim-5 | — | H3K9me3 | N. crassa | 331 | 1-331 (Rathert) | 331 | 77-331: preSET, |
| | | | | | | | | SET, postSET |
| Suvar3-9 | KYP | — | H3K9me1/2 | A. thaliana | 624 | 335-601 | 267 | — |
| (SUVH | | | | | | | (Jackson) | |
| subfamily) | | | | | | | | |
| Suvar3-9 | SUVR4 | H3K9me1 | H3K9me2/3 | A. thaliana | 492 | 180-492 | 313 | 192-462: preSET, |
| (SUVR | | | | | | | (Thorstensen) | SET, postSET |
| subfamily) | | | | | | | | |
| Suvar4-20 | SET4 | — | H4K20me3 | C. elegans | 288 | 1-288 (Vielle) | 288 | — |
| SET8 | SET1 | — | H4K20me1 | C. elegans | 242 | 1-242 (Vielle) | 242 | — |
| SET8 | SETD8 | — | H4K20me1 | H. sapiens | 393 | 185-393 | 209 | 256-382: SET |
| | | | | | | | (Couture) | |
| SET8 | TgSET8 | — | H4K20me1/2/3 | T. gondii | 1893 | 1590-1893 (Sautel) | 304 | 1749-1884: SET |

In some embodiment, the functional domain may be a Histone Methyltransferase (HMT) Recruiter Effector Domain. Preferred examples include those in the Table below, namely Hpla, PHF19, and NIPP1.

Histone Methyltransferase (HMT) Recruiter Effector Domains

| | | | | | Full | Selected | | |
|---|---|---|---|---|---|---|---|---|
| Subtype/ | | Substrate | Modification (if | | size | truncation | | |
| Complex | Name | (if known) | known) | Organism | (aa) | (aa) | Final size (aa) | Catalytic domain |
| — | Hp1a | — | H3K9me3 | M. musculus | 191 | 73-191 | 119 | 121-179: |
| | | | | | | | (Hathaway) | chromoshadow |
| — | PHF19 | — | H3K27me3 | H. sapiens | 580 | (1-250) + | 335 | 163-250: PHD2 |
| | | | | | | GGSG | (Ballaré) | |
| | | | | | | linker + | | |
| | | | | | | (500-580) | | |
| — | NIPP1 | — | H3K27me3 | H. sapiens | 351 | 1-329 (Jin) | 329 | 310-329: EED |

In some embodiment, the functional domain may be Histone Acetyltransferase Inhibitor Effector Domain. Preferred examples include SET/TAF-1B listed in the Table below.

Histone Acetyltransferase Inhibitor Effector Domains

| | | | | | Full | Selected | Final | |
|---|---|---|---|---|---|---|---|---|
| Subtype/ | | Substrate | Modification | | size | truncation | size | Catalytic |
| Complex | Name | (if known) | (if known) | Organism | (aa) | (aa) | (aa) | domain |
| — | SET/TAF-1β | — | — | M. musculus | 289 | 1-289 | 289 | — |
| | | | | | | (Cervoni) | | |

It is also preferred to target endogenous (regulatory) control elements (such as enhancers and silencers) in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g., by tiling the region of the putative control element as well as 200 bp up to 100 kB around the element) can be used as a means to verify such elements (by measuring the transcription of the gene of interest) or to detect novel control elements (e.g., by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g., a set of genes located in closest proximity to the control element) or b) whole-transcriptome readout by e.g., RNAseq or microarray. This would allow for the identification of likely candidate genes involved in the disease phenotype. Such candidate genes could be useful as novel drug targets.

Histone acetyltransferase (HAT) inhibitors are mentioned herein. However, an alternative, in example embodiments, is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the guide being directed to an epigenomic target sequence. Epigenomic target sequence may include, in example embodiments, include a promoter, silencer or an enhancer sequence.

Use of a functional domain linked to a Cas effector protein as described herein, preferably a dead-Cas effector protein, more preferably a dead-FnCas effector protein, to target epigenomic sequences can be used to activate or repress promoters, silencer or enhancers. Examples of acetyltransferases are known but may include, in example embodiments, histone acetyltransferases. In example embodiments, the histone acetyltransferase may comprise the catalytic core of the human acetyltransferase p300 (Gerbasch & Reddy, Nature Biotech 6Apr. 2015).

### Destabilized Cas and Fusion Proteins

In certain embodiments, the Cas5-HNH or Cas8-HNH polypeptide according to the invention as described herein is associated with or fused to a destabilization domain (DD). In example embodiments, the DD is ER50. A corresponding stabilizing ligand for this DD is, In example embodiments, 4HT. As such, in example embodiments, one of the at least one DDs is ER50 and a stabilizing ligand therefor is 4HT or CMP8. In example embodiments, the DD is DHFR50. A corresponding stabilizing ligand for this DD is, in example embodiments, TMP. As such, in example embodiments, one of the at least one DDs is DHFR50 and a stabilizing ligand therefor is TMP. In example embodiments, the DD is ER50. A corresponding stabilizing ligand for this DD is, in example embodiments, CMP8. CMP8 may therefore be an alternative stabilizing ligand to 4HT in the ER50 system. While it may be possible that CMP8 and 4HT can/should be used in a competitive matter, some cell types may be more susceptible to one or the other of these two ligands, and from this disclosure and the knowledge in the art the skilled person can use CMP8 and/or 4HT.

In example embodiments, one or two DDs may be fused to the N-terminal end of the Cas with one or two DDs fused to the C-terminal of the Cas. In example embodiments, the at least two DDs are associated with the Cas and the DDs are the same DD, i.e., the DDs are homologous. Thus, both (or two or more) of the DDs could be ER50 DDs. This is preferred in example embodiments. Alternatively, both (or two or more) of the DDs could be DHFR50 DDs. This is also preferred in example embodiments. In example embodiments, the at least two DDs are associated with the Cas and the DDs are different DDs, i.e., the DDs are heterologous. Thus, one of the DDS could be ER50 while one or more of the DDs or any other DDs could be DHFR50. Having two or more DDs which are heterologous may be advantageous as it would provide a greater level of degradation control. A tandem fusion of more than one DD at the N or C-term may enhance degradation; and such a tandem fusion can be, for example ER50-ER50-Cas or DHFR-DHFR-Cas It is envisaged that high levels of degradation would occur in the absence of either stabilizing ligand, intermediate levels of degradation would occur in the absence of one stabilizing ligand and the presence of the other (or another) stabilizing ligand, while low levels of degradation would occur in the presence of both (or two of more) of the stabilizing ligands. Control may also be imparted by having an N-terminal ER50 DD and a C-terminal DHFR50 DD.

In example embodiments, the fusion of the Cas with the DD comprises a linker between the DD and the Cas. In example embodiments, the linker is a GlySer linker. In example embodiments, the DD-Cas further comprises at least one Nuclear Export Signal (NES). In example embodiments, the DD-Cas comprises two or more NESs. In example embodiments, the DD-Cas comprises at least one Nuclear Localization Signal (NLS). This may be in addition to an NES. In example embodiments, the Cas comprises or consists essentially of or consists of a localization (nuclear import or export) signal as, or as part of, the linker between the Cas and the DD. HA or Flag tags are also within the ambit of the invention as linkers. Applicants use NLS and/or NES as linker and also use Glycine Serine linkers as short as GS up to (GGGGS)₃ (SEQ ID NO: 33).

Destabilizing domains have general utility to confer instability to a wide range of proteins; see, e.g., Miyazaki, J Am Chem Soc. Mar. 7, 2012; 134 (9): 3942-3945, incorporated herein by reference. CMP8 or 4-hydroxytamoxifen can be destabilizing domains. More generally, a temperature-sensitive mutant of mammalian DHFR (DHFRts), a destabilizing residue by the N-end rule, was found to be stable at a permissive temperature but unstable at 37° C. The addition of methotrexate, a high-affinity ligand for mammalian DHFR, to cells expressing DHFRts inhibited degradation of the protein partially. This was an important demonstration that a small molecule ligand can stabilize a protein otherwise targeted for degradation in cells. A rapamycin derivative was used to stabilize an unstable mutant of the FRB domain of mTOR (FRB*) and restore the function of the fused kinase, GSK-3β.6,7 This system demonstrated that ligand-dependent stability represented an attractive strategy to regulate the function of a specific protein in a complex biological environment. A system to control protein activity can involve the DD becoming functional when the ubiquitin complementation occurs by rapamycin induced dimerization of FK506-binding protein and FKBP12. Mutants of human FKBP12 or ecDHFR protein can be engineered to be metabolically unstable in the absence of their high-affinity ligands, Shield-1 or trimethoprim (TMP), respectively. These mutants are some of the possible destabilizing domains (DDs) useful in the practice of the invention and instability of a DD as a fusion with a Cas confers to the Cas degradation of the entire fusion protein by the proteasome. Shield-1 and TMP bind to and stabilize the DD in a dose-dependent manner. The estrogen receptor ligand binding domain (ERLBD, residues 305-549 of ERS1) can also be engineered as a destabilizing domain. Since the estrogen receptor signaling pathway is involved in a variety of diseases such as breast cancer, the pathway has been widely studied and numerous agonist and antagonists of estrogen receptor have been developed. Thus, compatible pairs of ERLBD and drugs are known. There are ligands that bind to mutant but not wild-type forms of the ERLBD. By using one of these mutant domains encoding three mutations (L384M, M421G, G521R) 12, it is possible to regulate the stability of an ERLBD-derived DD using a ligand that does not perturb endogenous estrogen-sensitive networks. An additional mutation (Y537S) can be introduced to further destabilize the ERLBD and to configure it as a potential DD candidate. This tetra-mutant is an advantageous DD development. The mutant ERLBD can be fused to a Cas and its stability can be regulated or perturbed using a ligand, whereby the Cas has a DD. Another DD can be a 12-kDa (107-amino-acid) tag based on a mutated FKBP protein, stabilized by Shield1 ligand; see, e.g., Nature Methods 5, (2008). For instance, a DD can be a modified FK506 binding protein 12 (FKBP12) that binds to and is reversibly stabilized by a synthetic, biologically inert small molecule, Shield-1; see, e.g., Banaszynski L A, Chen L C, Maynard-Smith L A, Ooi A G, Wandless T J. A rapid, reversible, and tunable method to regulate protein function in living cells using synthetic small molecules. Cell. 2006; 126:995-1004; Banaszynski L A, Sellmyer M A, Contag C H, Wandless T J, Thorne S H. Chemical control of protein stability and function in living mice. Nat Med. 2008; 14:1123-1127; Maynard-Smith L A, Chen L C, Banaszynski L A, Ooi A G, Wandless T J. A directed approach for engineering conditional protein stability using biologically silent small molecules. The Journal of biological chemistry. 2007; 282:24866-24872; and Rodriguez, Chem Biol. Mar. 23, 2012; 19 (3): 391-398—all of which are incorporated herein by reference and may be employed in the practice of the invention in selected a DD to associate with a Cas in the practice of this invention. As can be seen, the knowledge in the art includes a number of DDs, and the DD can be associated with, e.g., fused to, advantageously with a linker, to a Cas, whereby the DD can be stabilized in the presence of a ligand and when there is the absence thereof the DD can become destabilized, whereby the Cas is entirely destabilized, or the DD can be stabilized in the absence of a ligand and when the ligand is present the DD can become destabilized; the DD allows the Cas and hence the CRISPR-Cas complex or system to be regulated or controlled-turned on or off so to speak, to thereby provide means for regulation or control of the system, e.g., in an in vivo or in vitro environment. For instance, when a protein of interest is expressed as a fusion with the DD tag, it is destabilized and rapidly degraded in the cell, e.g., by proteasomes. Thus, absence of stabilizing ligand leads to a D associated Cas being degraded. When a new DD is fused to a protein of interest, its instability is conferred to the protein of interest, resulting in the rapid degradation of the entire fusion protein. Peak activity for Cas is sometimes beneficial to reduce off-target effects. Thus, short bursts of high activity are preferred. The present invention is able to provide such peaks. In some senses the system is inducible. In some other senses, the system repressed in the absence of stabilizing ligand and de-repressed in the presence of stabilizing ligand.

### Translation Activation and Repression

In an example embodiment, the functional domain has translation activation activity. For example, translation activation activity may comprise amino acid activation, which comprises attaching an amino acid to a transfer RNA (tRNA). A functional domain having translation activation activity may comprise aminoacyl tRNA synthetase. The translation activation activity of aminoacyl tRNA synthetase comprises binding ATP to an amino acid forming an AMP-amino acid intermediate and releasing pyrophosphate. Aminoacyl tRNA synthetase then binds the AMP-amino acid to a tRNA thereby releasing the AMP. The tRNA labeled amino acid is now activated.

In an example embodiment, the functional domain has translation repression activity. For example, a functional domain having translation repression activity may comprise a single stranded RNA (e.g., microRNA (miRNA)) or double stranded RNA (e.g., small interfering RNA (siRNA)) and/or an argonaute (Ago) protein. The combination of RNA and Ago protein may form an RNA induced silencing complex (RISC). A functional domain comprising an RISC may target messenger RNA (mRNA) complementary to the single or double stranded RNA and cleave the mRNA with the Ago, thus providing translation repression activity.

### Translation Release Factor

In an example embodiment, the functional domain has translation release factor activity. A functional domain having release factor activity, for example, may allow for termination of translation. A functional domain having release factor activity may comprise Class 1 (e.g., release factor 1 or release factor 2) or Class 2 release factors (e.g., release factor 3 (GTPase)). Release factors terminate translation by recognizing a stop codon (e.g., termination codon) in an mRNA sequence.

### Chromatin Remodeling Domains

In an example embodiment, the functional domain has chromatin remolding activity. A functional domain having chromatin remodeling activity, for example, may allow for histone modifications and/or moving, ejecting, or restructuring nucleosomes. A functional domain having chromatin remolding activity may comprise of, for example, histone acetyltransferases, deacetylases, methyltransferases, kinases, and ATP-dependent chromatin remolding. Chromatin remodeling domains alter the structure, composition, and positioning of nucleosomes to allow access of nucleosome DNA.

### Detectable Domains

In an example embodiment, the functional domain has detectable activity. A functional domain having detectable activity, for example, allows for the polypeptide of the present invention to be detected in a corresponding assay. A functional domain having detectable activity may comprise of fluorescent labeling (e.g., fluorescent dyes), radiolabeling (e.g., 35S-Methionine), magnetic labeling (e.g., magnetic beads), and/or affinity labeling (e.g., biotin-streptavidin).

### Polynucleotides Encoding CRISPR-Cas Systems and Vectors

The systems herein may comprise one or more polynucleotides. The polynucleotide(s) may comprise coding sequences of components of the systems herein, e.g., Cas5-HNH or Cas8-HINH polypeptide guide sequence RNA(s), functional domain(s), donor polynucleotide(s), and/or other components in the systems. The present disclosure further provides vectors or vector systems comprising one or more polynucleotides herein. The vectors or vector systems include those described in the delivery sections herein.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild-type” is a term of the art understood by skilled persons and means the typical form of an organism, strain, gene or characteristic as it occurs in nature as distinguished from mutant or variant forms. A “wild-type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides, mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. “Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence. As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life-eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

In an embodiment, the polynucleotide sequence is recombinant DNA. In further embodiments, the polynucleotide sequence further comprises additional sequences as described elsewhere herein. In an embodiment, the nucleic acid sequence is synthesized in vitro.

The present disclosure provides polynucleotide molecules that encode one or more components of the system or Cas5-HNH or Cas8-HNH polypeptide nuclease as referred to in any embodiment herein. In an embodiment, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a tol2 vector. In an embodiment, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In an embodiment, the 5′ and/or 3′ end of the isolated polynucleotide sequence may be modified to improve the stability of the sequence of actively avoid degradation. In an embodiment, the isolated polynucleotide sequence may be comprised in a bacteriophage. In other embodiments, the isolated polynucleotide sequence may be contained in agrobacterium species. In an embodiment, the isolated polynucleotide sequence is lyophilized.

Aspects of the invention relate to polynucleotide molecules that encode one or more components of one or more systems as described in any of the embodiments herein, wherein at least one or more regions of the polynucleotide molecule may be codon optimized for expression in eukaryotic cells. In an embodiment, the polynucleotide molecules that encode one or more components of one or more systems as described in any of the embodiments herein are optimized for expression in a mammalian cell or a plant cell.

An example of a codon optimized sequence is in this instance a sequence optimized for expression in a eukaryote, e.g., humans (i.e., being optimized for expression in humans), or for another eukaryote, animal or mammal as herein discussed. In one embodiment, an enzyme coding sequence encoding a DNA/RNA-targeting Cas5-HNH or Cas8-HNH polypeptide is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In one embodiment, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In one embodiment, one or more codons (e.g., 1, 2, 3, 4, 5, 10, 15, 20, 25, 50, or more, or all codons) in a sequence encoding a Cas5-HNH or Cas8-HNH polypeptide nuclease corresponds to the most frequently used codon for a particular amino acid.

### Delivery Systems

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs to of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties and can be adapted for use with the Cas5-HNH or Cas8-HNH polypeptides disclosed herein.

In one embodiment, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February;9 (1): 11-9; Klein R M, et al., Biotechnology. 1992; 24:384-6; Casas A M et al., Proc Natl Acad Sci USA. 1993 Dec. 1; 90 (23): 11212-11216; and U.S. Pat. No. 5,563,055, Davey M R et al., Plant Mol Biol. 1989 September; 13 (3): 273-85, which are incorporated by reference herein in their entireties.

The example delivery compositions, systems, and methods described herein related to a composition or Cas5-HINH or Cas8-HNH polypeptide also apply to functional domains and other components (e.g., other proteins and polynucleotides related to the Cas5-HNH or Cas8-HNH polypeptide such as reverse transcriptase, nucleotide deaminase, retrotransposon, donor polynucleotide, etc.).

### Cargos

The delivery systems may comprise one or more cargos. The cargos may comprise one or more components of the systems and compositions herein. A cargo may comprise one or more of the following: i) a plasmid encoding one or more proteins components in the compositions and systems such as the Cas5-HNH or Cas8-HNH polypeptide and/or functional domains; ii) a plasmid encoding one or more guide sequences, iii) mRNA of one or more one or more proteins components in the compositions and systems such as the Cas5-HNH or Cas8-HNH polypeptide and/or functional domains; iv) one or more guide sequence RNAs; v) one or more proteins components in the compositions and systems such as the Cas5-HNH or Cas8-HNH polypeptide and/or functional domains; vi) any combination thereof. The one or more protein components may include the nuclei acid-guided nuclease (e.g., Cas), reverse transcriptase, nucleotide deaminase, retrotransposon protein, other functional domain, or any combination thereof.

In some examples, a cargo may comprise a plasmid encoding one or more proteins components in the compositions and systems such as the Cas5-HNH or Cas8-HNH polypeptide and/or functional domains and one or more (e.g., a plurality of) guide sequence RNAs. In some cases, the plasmid may also encode a recombination template (e.g., for HDR). In one embodiment, a cargo may comprise mRNA encoding one or more protein components and one or more guide or sequence RNAs.

In some examples, a cargo may comprise one or more protein components and one or more guide or guide sequence RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November;33 (11): 1162-4.

### Physical Delivery

In one embodiment, the cargos may be introduced to cells by physical delivery methods. Examples of physical methods include microinjection, electroporation, and hydrodynamic delivery. Both nucleic acid and proteins may be delivered using such methods. For example, one or more protein components may be prepared in vitro, isolated, (refolded, purified if needed), and introduced to cells.

### Microinjection

Microinjection of the cargo directly to cells can achieve high efficiency, e.g., above 90% or about 100%. In one embodiment, microinjection may be performed using a microscope and a needle (e.g., with 0.5-5.0 μm in diameter) to pierce a cell membrane and deliver the cargo directly to a target site within the cell. Microinjection may be used for in vitro and ex vivo delivery.

Plasmids comprising coding sequences for one or more protein components and/or mRNAs, and/or guide sequence RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery guide sequence directly to the nucleus and mRNA to the cytoplasm, e.g., facilitating translation and shuttling of one or more protein components to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using Cas5-HNH or Cas8-HNH.

### Electroporation

In one embodiment, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses, non-viral vehicles, and other delivery reagents described herein.

The delivery vehicles in accordance with the present invention may have a greatest dimension (e.g., diameter) of less than 100 microns (μm). In one embodiment, the delivery vehicles have a greatest dimension of less than 10 μm. In one embodiment, the delivery vehicles may have a greatest dimension of less than 2000 nanometers (nm). In one embodiment, the delivery vehicles may have a greatest dimension of less than 1000 nanometers (nm). In one embodiment, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In one embodiment, the delivery vehicles may have a greatest dimension ranging between 25 nm and 200 nm.

In one embodiment, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles). Nanoparticles may also be used to deliver the compositions and systems to plant cells, e.g., as described in International Patent Publication No. WO 2008042156, US Publication Application No. U.S. Pat. No. 2013/0185823, and International Patent Publication No WO 2015/089419.

### Vectors

The systems, compositions, and/or delivery systems may comprise one or more vectors. The present disclosure also includes vector systems. A vector system may comprise one or more vectors. In one embodiment, a vector refers to a nucleic acid molecule capable of transporting another nucleic acid to which it has been linked. Vectors include nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. A vector may be a plasmid, e.g., a circular double stranded DNA loop into which additional DNA segments can be inserted, such as by standard molecular cloning techniques. Certain vectors may be capable of autonomous replication in a host cell into which they are introduced (e.g., bacterial vectors having a bacterial origin of replication and episomal mammalian vectors). Some vectors (e.g., non-episomal mammalian vectors) are integrated into the genome of a host cell upon introduction into the host cell, and thereby are replicated along with the host genome. In certain examples, vectors may be expression vectors, e.g., capable of directing the expression of genes to which they are operatively-linked. In some cases, the expression vectors may be for expression in eukaryotic cells. Common expression vectors of utility in recombinant DNA techniques are often in the form of plasmids.

Examples of vectors include pGEX, pMAL, pRIT5, E. coli expression vectors (e.g., pTrc, pET 11d, yeast expression vectors (e.g., pYepSec1, pMFa, pJRY88, pYES2, and picZ, Baculovirus vectors (e.g., for expression in insect cells such as SF9 cells) (e.g., pAc series and the pVL series), mammalian expression vectors (e.g., pCDM8 and pMT2PC).

A vector may comprise i) one or more protein components encoding sequence(s), and/or ii) a single, or at least 2, at least 3, at least 4, at least 5, at least 6, at least 7, at least 8, at least 9, at least 10, at least 12, at least 14, at least 16, at least 32, at least 48, at least 50 guide RNA(s) encoding sequences. In a single vector there can be a promoter for each RNA coding sequence. Alternatively, or additionally, in a single vector, there may be a promoter controlling (e.g., driving transcription and/or expression) multiple RNA encoding sequences.

Furthermore, that compositions or systems may be delivered via a vector, e.g., a separate vector or the same vector that is encoding the complex. When provided by a separate vector, the RNA that targets Cas5-HNH or Cas8-HNH polypeptide expression can be administered sequentially or simultaneously. When administered sequentially, the RNA that targets Cas5-HNH or Cas8-HNH polypeptide expression is to be delivered after the RNA that is intended for e.g., gene editing or gene engineering. This period may be a period of minutes (e.g., 5 minutes, 10 minutes, 20 minutes, 30 minutes, 45 minutes, 60 minutes). This period may be a period of hours (e.g., 2 hours, 4 hours, 6 hours, 8 hours, 12 hours, 24 hours). This period may be a period of days (e.g., 2 days, 3 days, 4 days, 7 days). This period may be a period of weeks (e.g., 2 weeks, 3 weeks, 4 weeks). This period may be a period of months (e.g., 2 months, 4 months, 8 months, 12 months). This period may be a period of years (2 years, 3 years, 4 years). In this fashion, the Cas5-HNH or Cas8-HNH polypeptide nuclease associates with a first guide sequence RNA capable of hybridizing to a first target, such as a genomic locus or loci of interest and undertakes the function(s) desired of the system (e.g., gene engineering); and subsequently the Cas5-HNH or Cas8-HNH polypeptide may then associate with the second guide sequence RNA capable of hybridizing to the sequence comprising at least part of the Cas5-HNH or Cas8-HNH polypeptide. Where the guide RNA targets the sequences encoding expression of the Cas5-HNH or Cas8-HNH polypeptide, the enzyme becomes impeded and the system becomes self-inactivating. In the same manner, RNA that targets Cas5-HNH or Cas8-HNH polypeptide expression applied via, for example liposome, lipofection, particles, microvesicles as explained herein, may be administered sequentially or simultaneously. Similarly, self-inactivation may be used for inactivation of one or more guide sequence RNA used to target one or more targets.

### Vector Regulatory Elements

A vector may comprise one or more regulatory elements. The regulatory element(s) may be operably linked to coding sequences of Cas5-HNH or Cas8-HNH polypeptide, accessory proteins, guide sequence RNA or combination thereof. The term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). In certain examples, a vector may comprise: a first regulatory element operably linked to a nucleotide sequence encoding a Cas5-HNH or Cas8-HNH polypeptide, and a second regulatory element operably linked to a nucleotide sequence encoding a guide sequence RNA.

Examples of regulatory elements include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific.

Examples of promoters include one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, the phosphoglycerol kinase (PGK) promoter, and the EF1a promoter.

### Viral Vectors

The cargos may be delivered by viruses. In one embodiment, viral vectors are used. A viral vector may comprise virally-derived DNA or RNA sequences for packaging into a virus (e.g., retroviruses, replication defective retroviruses, adenoviruses, replication defective adenoviruses, and adeno-associated viruses). Viral vectors also include polynucleotides carried by a virus for transfection into a host cell. Viruses and viral vectors may be used for in vitro, ex vivo, and/or in vivo deliveries.

### Adeno-Associated Viruses (AAV)

The systems and compositions herein may be delivered by adeno associated virus (AAV). AAV vectors may be used for such delivery. AAV, of the Dependovirus genus and Parvoviridae family, is a single stranded DNA virus. In one embodiment, AAV may provide a persistent source of the provided DNA, as AAV delivered genomic material can exist indefinitely in cells, e.g., either as exogenous DNA or, with some modification, be directly integrated into the host DNA. In one embodiment, AAV do not cause or relate with any diseases in humans. The virus itself is able to efficiently infect cells while provoking little to no innate or adaptive immune response or associated toxicity.

Examples of AAV that can be used herein include AAV-1, AAV-2, AAV-3, AAV-4, AAV-5, AAV-6, AAV-8, and AAV-9. The type of AAV may be selected with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. AAV-2-based vectors were originally proposed for CFTR delivery to CF airways, other serotypes such as AAV-1, AAV-5, AAV-6, and AAV-9 exhibit improved gene transfer efficiency in a variety of models of the lung epithelium. Examples of cell types targeted by AAV are described in Grimm, D. et al, J. Virol. 82:5887-5911 (2008)), and shown in table below:

Examples of cell types targeted by AAV

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

The AAV particles may be created in HEK 293 T cells. Once particles with specific tropism have been created, they are used to infect the target cell line much in the same way that native viral particles do. This may allow for persistent presence of the components in the infected cell type, and what makes this version of delivery particularly suited to cases where long-term expression is desirable. Examples of doses and formulations for AAV that can be used include those describe in U.S. Pat. Nos. 8,454,972 and 8,404,658.

Various strategies may be used for delivery the systems and compositions herein with AAVs. In some examples, coding sequences of Cas5-HNH or Cas8-HNH polypeptide nuclease and guide RNA may be packaged directly onto one DNA plasmid vector and delivered via one AAV particle. In some examples, AAVs may be used to deliver guide sequence RNA into cells that have been previously engineered to express Cas5-HINH or Cas8-HNH polypeptide. In some examples, coding sequences of Cas5-HNH or Cas8-HNH polypeptide and guide or sequence RNA may be made into two separate AAV particles, which are used for co-transfection of target cells. In some examples, markers, tags, and other sequences may be packaged in the same AAV particles as coding sequences of Cas5-HNH or Cas8-HNH polypeptide and/or guide sequence RNAs.

### Lentiviruses

The systems and compositions herein may be delivered by lentiviruses. Lentiviral vectors may be used for such delivery. Lentiviruses are complex retroviruses that have the ability to infect and express their genes in both mitotic and post-mitotic cells.

Examples of lentiviruses include human immunodeficiency virus (HIV), which may use its envelope glycoproteins of other viruses to target a broad range of cell types; minimal non-primate lentiviral vectors based on the equine infectious anemia virus (EIAV), which may be used for ocular therapies. In an embodiment, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2: 36ra43) may be used/and or adapted to the nucleic acid-targeting system herein.

Lentiviruses may be pseudo-typed with other viral proteins, such as the G protein of vesicular stomatitis virus. In doing so, the cellular tropism of the lentiviruses can be altered to be as broad or narrow as desired. In some cases, to improve safety, second- and third-generation lentiviral systems may split essential genes across three plasmids, which may reduce the likelihood of accidental reconstitution of viable viral particles within cells.

In some examples, leveraging the integration ability, lentiviruses may be used to create libraries of cells comprising various genetic modifications, e.g., for screening and/or studying genes and signaling pathways.

### Adenoviruses

The systems and compositions herein may be delivered by adenoviruses. Adenoviral vectors may be used for such delivery. Adenoviruses include nonenveloped viruses with an icosahedral nucleocapsid containing a double stranded DNA genome. Adenoviruses may infect dividing and non-dividing cells. In one embodiment, adenoviruses do not integrate into the genome of host cells, which may be used for limiting off-target effects of systems in gene editing applications.

Viral Vehicles for Delivery into Plants

The systems and compositions may be delivered to plant cells using viral vehicles. In one embodiment, the compositions and systems may be introduced in the plant cells using a plant viral vector (e.g., as described in Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

### Non-Viral Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, gold nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs), liposomes, and stable nucleic-acid-lipid particles.

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of Cas5-HNH or Cas8-HNH polypeptide and/or guide sequence RNA) and/or RNA molecules (e.g., mRNA of Cas5-HNH or Cas8-HNH polypeptide guide sequence RNAs). In certain cases, LNPs may be use for delivering RNP complexes of Cas5-HNH or Cas8-HNH polypeptide or guide sequence RNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-O-[2″-(methoxypolyethyleneglycol 2000) succinoyl]-1,2-dimyristoyl-sn-glycol (PEG-S-DMG), R-3-[(ro-methoxy-poly(ethylene glycol) 2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-220 December 2011).

### Liposomes

In one embodiment, a lipid particle may be liposome. Liposomes are spherical vesicle structures composed of a uni- or multilamellar lipid bilayer surrounding internal aqueous compartments and a relatively impermeable outer lipophilic phospholipid bilayer. In one embodiment, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

### Stable Nucleic-Acid-Lipid Particles (SNALPs)

In one embodiment, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some comprise examples, SNALPs may synthetic cholesterol, dipalmitoylphosphatidylcholine, 3-N-[(w-methoxy polyethylene glycol) 2000) carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-CDMA, and 1,2-dilinoleyloxy-3-(N;N-dimethyl)aminopropane (DLinDMA).

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

### Lipoplexes/Polyplexes.

In one embodiment, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2p (e.g., forming DNA/Ca2+ microcomplexes), polyethenimine (PEI) (e.g., branched PEI), and poly(L-lysine) (PLL).

### Cell Penetrating Peptides

In one embodiment, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin β3 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to the Cas5-HNH or Cas8-HNH polypeptide nuclease directly, which is then complexed with the hRNA and delivered to cells. In some examples, separate delivery of CPP-Cas5-HNH or Cas8-HNH and CPP-hRNA to multiple cells may be performed. CPP may also be used to delivery RNPs.

CPPs may be used to deliver the compositions and systems to plants. In some examples, CPPs may be used to deliver the components to plant protoplasts, which are then regenerated to plant cells and further to plants.

### DNA Nanoclews

In one embodiment, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22;136 (42): 14722-5; and Sun W et al, Angew Chem Int Ed Engl. 2015 Oct. 5;54 (41): 12029-33. DNA nanoclew may have a palindromic sequences to be partially complementary to the guide RNA within the Cas5-HNH or Cas8-HNH polypeptide nuclease:hRNA ribonucleoprotein complex. A DNA nanoclew may be coated, e.g., coated with PEI to induce endosomal escape.

### Gold Nanoparticles

In one embodiment, the delivery vehicles comprise gold nanoparticles (also referred to AuNPs or colloidal gold). Gold nanoparticles may form complex with cargos, e.g., Cas5-HINH or Cas8-HNH polypeptide nuclease:hRNA RNP. Gold nanoparticles may be coated, e.g., coated in a silicate and an endosomal disruptive polymer, PAsp (DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901.

iTOP

In one embodiment, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In one embodiment, the delivery vehicles may comprise polymer-based particles (e.g., nanoparticles). In one embodiment, the polymer-based particles may mimic a viral mechanism of membrane fusion. The polymer-based particles may be a synthetic copy of Influenza virus machinery and form transfection complexes with various types of nucleic acids (siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In one embodiment, the polymer-based particles may comprise alkylated and carboxyalkylated branched polyethylenimine. In some examples, the polymer-based particles are VIROMER, e.g., VIROMER RNAi, VIROMER RED, VIROMER mRNA. Example methods of delivering the systems and compositions herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data., doi: 10.13140/RG.2.2.23912.16642.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo loading capacities. In one embodiment, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January;267 (1): 9-21; El-Andaloussi S, et al., Nat Protoc. 2012 December;7 (12): 2112-26; Uno Y, et al., Hum Gene Ther. 2011 June;22 (6): 711-9; Zou W, et al., Hum Gene Ther. 2011 April;22 (4): 465-75.

In some examples, the exosome may form a complex (e.g., by binding directly or indirectly) to one or more components of the cargo. In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

### Retrovirus Like Delivery Systems

The delivery vehicle may comprise a retro-virus like protein, such as PEG10, which is capable of incorporating a cargo into a virus-like particle. As such systems can be re-programmed to package specific cargos, polynucleotides encoding composnents of the IsrB systems disclosed herein may be further modified with a recognition sequence that leads to selective packaging of the IsrB components into such retro-virus like VLPs. Said VLPs may be further modified with fusogenic proteins that impart tissue or cell specificity. Example systems are disclosed in Segal et al. Mammalian retrovirus-like protein PEG10 packages its own mRNA and can be pseudotyped for mRNA delivery. 373 Science, 882-889 (2021), which is incorporated herein by reference.

### Genetically-Modified Cells and Organisms

The present disclosure further provides cells comprising one or more components of the compositions and systems herein, e.g., the Cas5-HNH or Cas8-HNH polypeptide and/or guide sequence RNAs. Also provided include cells modified by the systems and methods herein, and cell cultures, tissues, organs, organism comprising such cells or progeny thereof. In one embodiment, the present disclosure provides a method of modifying a cell or organism. The cell may be a prokaryotic cell or an eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may be a therapeutic T cell or antibody-producing B-cell. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

In one embodiment, one or more polynucleotide molecules, vectors, or vector systems driving expression of one or more elements of the compositions, systems, or delivery systems comprising one or more elements of the nucleic acid-targeting system are introduced into a host cell such that expression of the elements of the nucleic acid-targeting system direct formation of a nucleic acid-targeting complex at one or more target sites. In an embodiment of the invention the host cell may be a eukaryotic cell, a prokaryotic cell, or a plant cell.

In one embodiment, the host cell is a cell of a cell line. Cell lines are available from a variety of sources known to those with skill in the art (see, e.g., the American Type Culture Collection (ATCC) (Manassas, Va.)). In one embodiment, a cell transfected with one or more vectors described herein is used to establish a new cell line comprising one or more vector-derived sequences. In one embodiment, a cell transiently transfected with the components of a system as described herein (such as by transient transfection of one or more vectors, or transfection with RNA), and modified through the activity of a complex, is used to establish a new cell line comprising cells containing the modification but lacking any other exogenous sequence. In one embodiment, cells transiently or non-transiently transfected with one or more vectors described herein, or cell lines derived from such cells are used in assessing one or more test compounds.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present invention are provided, including (isolated) stem cells, and progeny thereof.

In an embodiment, the plants or non-human animals comprise at least one of the system components, polynucleotide molecules, vectors, vector systems, or cells described in any of the embodiments herein at least one tissue type of the plant or non-human animal. In an embodiment, non-human animals comprise at least one of the system components, polynucleotide molecules, vectors, vector systems, or cells described in any of the embodiments herein in at least one tissue type. In an embodiment, the presence of the system components is transient, in that they are degraded over time. In an embodiment, expression of the components of the systems and compositions described in any of the embodiments comprised in polynucleotide molecules, vectors, vector systems, or cells is limited to certain tissue types or regions in the plant or non-human animal. In an embodiment, the expression of the components of the systems and compositions described in any of the embodiments comprised in polynucleotide molecules, vectors, vector systems, or cells is dependent of a physiological cue. In an embodiment, expression of the components of the systems and compositions described in any of the embodiments comprised in polynucleotide molecules, vectors, vector systems, or cells may be triggered by an exogenous molecule. In an embodiment, expression of the components of the systems and compositions described in any of the embodiments comprised in polynucleotide molecules, vectors, vector systems, or cells is dependent on the expression of a non-cas molecule in the plant or non-human animal.

### Applications and Uses in General

The systems, the vector systems, the vectors and the compositions described herein may be used in various nucleic acids-targeting applications, altering or modifying synthesis of a gene product, such as a protein, nucleic acids cleavage, nucleic acids editing, nucleic acids splicing; trafficking of target nucleic acids, tracing of target nucleic acids, isolation of target nucleic acids, visualization of target nucleic acids, etc.

Aspects of the invention thus also encompass methods and uses of the compositions and systems described herein in genome engineering, e.g., for altering or manipulating the expression of one or more genes or the one or more gene products, in prokaryotic or eukaryotic cells, in vitro, in vivo or ex vivo. In some examples, the target polynucleotides are target sequences within genomic DNA, including nuclear genomic DNA, mitochondrial DNA, or chloroplast DNA.

Typically, in the context of a nucleic acid-targeting system, formation of a nucleic acid-targeting complex (comprising a guide sequence RNA hybridized to a target sequence and complexed with one or more nucleic acid-targeting effector proteins) results in cleavage of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g., within 1, 2, 3, 4, 5, 6, 7, 8, 9, 10, 20, 50, or more base pairs from the target sequence, wherein the target sequence is comprised within a target locus of interest).

In one embodiment, the present disclosure provides a method of targeting a polynucleotide, comprising contacting a sample (such as cell, population of cells, tissue, organ, or an organism) that comprises a target polynucleotide with the composition, systems, polynucleotide(s), or vector(s). The contacting may result in modification of a gene product or modification of the amount or expression of a gene product. In some examples, the target sequence of the polynucleotide is a disease-associated target sequence.

In one embodiment, the present disclosure provides a method of modifying target polynucleotides comprising delivering the composition, the one or more polynucleotides of 2, or one or more vectors to a cell or population of cells comprising the target polynucleotides, wherein the complex directs the reverse transcriptase to the target sequence and the reverse transcriptase facilitates insertion of the donor sequence from the guide sequence RNA into the target polynucleotide.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown and may be at a normal or abnormal level.

The target polynucleotide of a complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). Without wishing to be bound by theory, it is believed that the target sequence should be associated with a PAM (proto-adjacent motif); that is, a short sequence recognized by the complex. The precise sequence and length requirements for the PAM differ depending on the Cas5-HNH or Cas8-HNH polypeptide used, but PAMs are typically 2-5 base pair sequences adjacent the protospacer (that is, the target sequence) A skilled person will be able to identify further PAM sequences for use with a given Cas5-HNH or Cas8-HNH polypeptide. Further, engineering of the PAM Interacting domain may allow programing of PAM specificity, improve target site recognition fidelity, and increase the versatility of the Cas5-HNH or Cas8-HNH polypeptide nuclease, genome engineering platform. Cas5-HNH or Cas8-HNH polypeptide may be engineered to alter their PAM specificity.

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown and may be at a normal or abnormal level.

Aspects of the invention relate to a method of targeting a polynucleotide, comprising contacting a sample that comprises the polynucleotide with a composition, system or Cas5-HNH or Cas8-HINH polypeptide as described in any embodiment herein, a delivery system comprising a composition, system or Cas5-HNH or Cas8-HNH polypeptide as described in any embodiment herein, a polynucleotide comprising a composition, system or Cas5-HNH or Cas8-HNH as described in any embodiment herein, a vector comprising a composition, system or Cas5-HNH or Cas8-HNH polypeptide as described in any embodiment herein, or a vector system comprising a composition, system or Cas5-HNH or Cas8-HNH polypeptide as described in any embodiment herein. In an embodiment, a target polynucleotide is contacted with at least two different compositions, system or Cas5-HNH or Cas8-HNH polypeptide. In further embodiments, the two different Cas5-HNH or Cas8-HNH polypeptide nucleases have different target polynucleotide specificities, or degrees of specificity. In an embodiment, the two different Cas5-HNH or Cas8-HNH polypeptides have a different PAM specificity.

Also envisaged are methods of targeting a polynucleotide, comprising contacting a sample that comprises the polynucleotide with the composition and systems, vectors, polynucleotides, herein wherein contacting results in modification of a gene product or modification of the amount or expression of a gene product. In an embodiment, the expression of the targeted gene product is increased by the method. In an embodiment, the expression of the targeted gene product is increased by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100%. In an embodiment, the expression of the targeted gene product is increased at least 1.5-fold, at least 2-fold, at least 2.5-fold, at least 3-fold, at least 3.5-fold, at least 3.5-fold, at least 4-fold, at least 4.5-fold, at least 5-fold, at least 10-fold, at least 10-fold, at least 15-fold, at least 20-fold, at least 25-fold, at least 50-fold, at least 100-fold. In an embodiment, the expression of the targeted gene product is reduced by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, by at least 100%. In an embodiment, the expression of the targeted gene product is reduced at least 1.5-fold, at least 2-fold, at least 2.5-fold, at least 3-fold, at least 3.5-fold, at least 3.5-fold, at least 4-fold, at least 4.5-fold, at least 5-fold, at least 10-fold, at least 10-fold, at least 15-fold, at least 20-fold, at least 25-fold, at least 50-fold, at least 100-fold. In alternative embodiments, the expression of the targeted gene product is reduced by the method. In further embodiments, expression of the targeted gene may be completely eliminated, or may be considered eliminated as remnant expression levels of the targeted gene fall below the detection limit of methods known in the art that are used to quantify, detect, or monitor expression levels of genes.

In one embodiment, one or more polynucleotide molecules, vectors, or vector systems driving expression of one or more elements of a nucleic acid-targeting system or delivery systems comprising one or more elements of the nucleic acid-targeting system are introduced into a host cell such that expression of the elements of the nucleic acid-targeting system direct formation of a nucleic acid-targeting complex at one or more target sites. In an embodiment of the invention the host cell may be a eukaryotic cell, a prokaryotic cell, or a plant cell.

In one embodiment, the host cell is a cell of a cell line. Cell lines are available from a variety of sources known to those with skill in the art (see, e.g., the American Type Culture Collection (ATCC) (Manassas, Va.)). In one embodiment, a cell transfected with one or more vectors described herein is used to establish a new cell line comprising one or more vector-derived sequences. In one embodiment, a cell transiently transfected with the components of a composition or system as described herein (such as by transient transfection of one or more vectors, or transfection with RNA), and modified through the activity of a complex, is used to establish a new cell line comprising cells containing the modification but lacking any other exogenous sequence. In one embodiment, cells transiently or non-transiently transfected with one or more vectors described herein, or cell lines derived from such cells are used in assessing one or more test compounds.

Further intended are isolated human cells or tissues, plants or non-human animals comprising one or more of the polynucleotide molecules, vectors, vector systems, or cells described in any of the embodiments herein. In an aspect, host cells and cell lines modified by or comprising the compositions, systems or modified enzymes of present invention are provided, including (isolated) stem cells, and progeny thereof.

In an embodiment, the plants or non-human animals comprise at least one of the compositions, polynucleotide molecules, vectors, vector systems, or cells described in any of the embodiments herein at least one tissue type of the plant or non-human animal. In certain embodiment, non-human animals comprise at least one of the compositions, polynucleotide molecules, vectors, vector systems, or cells described in any of the embodiments herein in at least one tissue type. In an embodiment, the presence of the compositions is transient, in that they are degraded over time. In an embodiment, expression of the compositions described in any of the embodiments comprised in polynucleotide molecules, vectors, vector systems, or cells is limited to certain tissue types or regions in the plant or non-human animal. In an embodiment, the expression of the compositions described in any of the embodiments comprised in polynucleotide molecules, vectors, vector systems, or cells is dependent of a physiological cue. In an embodiment, expression of the compositions described in any of the embodiments comprised in polynucleotide molecules, vectors, vector systems, or cells may be triggered by an exogenous molecule. In an embodiment, expression of the compositions described in any of the embodiments comprised in polynucleotide molecules, vectors, vector systems, or cells is dependent on the expression of a non-Cas molecule in the plant or non-human animal.

In one aspect, the invention provides methods for using one or more elements of a nucleic acid-targeting system. The nucleic acid-targeting complex of the invention provides an effective means for modifying a target DNA or RNA (single or double stranded, linear or super-coiled). The nucleic acid-targeting complex of the invention has a wide variety of utility including modifying (e.g., deleting, inserting, translocating, inactivating, activating) a target DNA or RNA in a multiplicity of cell types. As such, the nucleic acid-targeting complex of the invention has a broad spectrum of applications in, e.g., gene therapy, drug screening, disease diagnosis, and prognosis. An exemplary nucleic acid-targeting complex comprises a DNA or RNA-targeting effector protein complexed with a guide sequence RNA hybridized to a target sequence within the target locus of interest.

In one embodiment, this invention provides a method of cleaving a target polynucleotide. The method may comprise modifying a target polynucleotide using a nucleic acid-targeting complex that binds to the target polynucleotide and effect cleavage of said target polynucleotide. In an embodiment, the nucleic acid-targeting complex of the invention, when introduced into a cell, may create a break (e.g., a single or a double strand break) in the polynucleotide sequence. For example, the method can be used to cleave a disease polynucleotide in a cell. For example, an exogenous template comprising a sequence to be integrated flanked by an upstream sequence and a downstream sequence may be introduced into a cell. The upstream and downstream sequences share sequence similarity with either side of the site of integration in the polynucleotide. The exogenous template comprises a sequence to be integrated (e.g., a mutated RNA). The sequence for integration may be a sequence endogenous or exogenous to the cell. Examples of a sequence to be integrated include polynucleotide encoding a protein or a non-coding RNA (e.g., a microRNA). Thus, the sequence for integration may be operably linked to an appropriate control sequence or sequences. Alternatively, the sequence to be integrated may provide a regulatory function. The upstream and downstream sequences in the recombination template are selected to promote recombination between the RNA sequence of interest and the recombination. The upstream sequence is a polynucleotide sequence that shares sequence similarity with the sequence upstream of the targeted site for integration. Similarly, the downstream sequence is a polynucleotide sequence that shares sequence similarity with the polynucleotide sequence downstream of the targeted site of integration. The upstream and downstream sequences in the recombination template can have 75%, 80%, 85%, 90%, 95%, or 100% sequence identity with the targeted sequence. Preferably, the upstream and downstream sequences in the recombination template have about 95%, 96%, 97%, 98%, 99%, or 100% sequence identity with the targeted sequence. In some methods, the upstream and downstream sequences in the recombination template have about 99% or 100% sequence identity with the targeted sequence. An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp. In some methods, the recombination template may further comprise a marker. Such a marker may make it easy to screen for targeted integrations. Examples of suitable markers include restriction sites, fluorescent proteins, or selectable markers. The recombination template of the invention can be constructed using recombinant techniques (see, for example, Sambrook et al., 2001 and Ausubel et al., 1996). In a method for modifying a target sequence by integrating a recombination template, a break (e.g., double or single stranded break in double or single stranded DNA or RNA) is introduced into the DNA or RNA sequence by the nucleic acid-targeting complex, the break is repaired via homologous recombination with a recombination template such that the template is integrated into the target. The presence of a double-stranded break facilitates integration of the template. In other embodiments, this invention provides a method of modifying expression of a RNA in a eukaryotic cell. The method comprises increasing or decreasing expression of a target polynucleotide by using a nucleic acid-targeting complex that binds to the DNA or RNA (e.g., mRNA or pre-mRNA). In some methods, a target can be inactivated to affect the modification of the expression in a cell. For example, upon the binding of a nucleic acid-targeting complex to a target sequence in a cell, the target is inactivated such that the sequence is not translated, the coded protein is not produced, or the sequence does not function as the wild-type sequence does. For example, a protein or microRNA coding sequence may be inactivated such that the protein or microRNA or pre-microRNA transcript is not produced. The target of a nucleic acid-targeting complex can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., ncRNA, lncRNA, tRNA, or rRNA). Examples of target RNA include a sequence associated with a signaling biochemical pathway, e.g., a signaling biochemical pathway-associated polynucleotide. Examples of target polynucleotide include a disease associated polynucleotide. A “disease-associated” polynucleotide refers to any polynucleotide which is yielding translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated polynucleotide also refers to a gene possessing mutation(s) or genetic variation that is directly responsible or is in linkage disequilibrium with a gene(s) that is responsible for the etiology of a disease. The translated products may be known or unknown and may be at a normal or abnormal level. The target RNA of a nucleic acid-targeting complex can be any polynucleotide endogenous or exogenous to the eukaryotic cell. For example, the target RNA can be a RNA residing in the nucleus of the eukaryotic cell. The target polynucleotide can be a sequence coding a gene product (e.g., a protein) or a non-coding sequence (e.g., ncRNA, lncRNA, tRNA, or rRNA).

In one embodiment, the method may comprise allowing a composition to bind to the target DNA or RNA to effect cleavage of said target DNA or RNA thereby modifying the target DNA or RNA, wherein the nucleic acid-targeting complex comprises a nucleic acid-targeting effector protein complexed with a guide RNA hybridized to a target sequence within said target DNA or RNA. In one aspect, the invention provides a method of modifying expression of DNA or RNA in a eukaryotic cell. In one embodiment, the method comprises allowing a nucleic acid-targeting complex to bind to the DNA or RNA such that said binding results in increased or decreased expression of said DNA or RNA; wherein the nucleic acid-targeting complex comprises a nucleic acid-targeting effector protein complexed with a guide RNA. Similar considerations and conditions apply as above for methods of modifying a target DNA or RNA. In fact, these sampling, culturing and re-introduction options apply across the aspects of the present invention. In one aspect, the invention provides for methods of modifying a target DNA or RNA in a eukaryotic cell, which may be in vivo, ex vivo or in vitro. In one embodiment, the method comprises sampling a cell or population of cells from a human or non-human animal and modifying the cell or cells. Culturing may occur at any stage ex vivo. The cell or cells may even be re-introduced into the non-human animal or plant. For re-introduced cells it is particularly preferred that the cells are stem cells. The compositions as described in any embodiment herein may be used to detect nucleic acid identifiers. Nucleic acid identifiers are non-coding nucleic acids that may be used to identify a particular article. Example nucleic acid identifiers, such as DNA watermarks, are described in Heider and Barnekow. “DNA watermarks: A proof of concept” BMC Molecular Biology 9:40 (2008). The nucleic acid identifiers may also be a nucleic acid barcode. A nucleic acid-based barcode is a short sequence of nucleotides (for example, DNA, RNA, or combinations thereof) that is used as an identifier for an associated molecule, such as a target molecule and/or target nucleic acid. A nucleic acid barcode can have a length of at least, for example, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80, 90, or 100 nucleotides, and can be in single- or double-stranded form. One or more nucleic acid barcodes can be attached, or “tagged,” to a target molecule and/or target nucleic acid. This attachment can be direct (for example, covalent or non-covalent binding of the barcode to the target molecule) or indirect (for example, via an additional molecule, for example, a specific binding agent, such as an antibody (or other protein) or a barcode receiving adaptor (or other nucleic acid molecule). Target molecule and/or target nucleic acids can be labeled with multiple nucleic acid barcodes in combinatorial fashion, such as a nucleic acid barcode concatemer. Typically, a nucleic acid barcode is used to identify target molecules and/or target nucleic acids as being from a particular compartment (for example a discrete volume), having a particular physical property (for example, affinity, length, sequence, etc.), or having been subject to certain treatment conditions. Target molecule and/or target nucleic acid can be associated with multiple nucleic acid barcodes to provide information about all of these features (and more). Methods of generating nucleic acid-barcodes are disclosed, for example, in International Patent Application Publication No. WO/2014/047561.

In an embodiment, compositions induce a double strand break for the purpose of inducing HDR-mediated correction. In a further embodiment, two or more guide sequence RNAs complexing with Cas5-HNH or Cas8-HNH polypeptide nuclease or an ortholog or homolog thereof, may be used to induce multiplexed breaks for purpose of inducing HDR-mediated correction.

A recombination template nucleic acid, as that term is used herein, refers to a nucleic acid sequence which can be used in conjunction with compositions discloser herein to alter the structure of a target position. In an embodiment, the target nucleic acid is modified to have some or all of the sequence of the recombination template nucleic acid, typically at or near cleavage site(s). In an embodiment, the recombination template nucleic acid is single stranded. In an alternate embodiment, the recombination template nucleic acid is double stranded. In an embodiment, the recombination template nucleic acid is DNA, e.g., double stranded DNA. In an alternate embodiment, the recombination template nucleic acid is single stranded DNA.

In one embodiment, a recombination template is provided to serve as a template in homologous recombination, such as within or near a target sequence nicked or cleaved by a nucleic acid-targeting effector protein as a part of a nucleic acid-targeting complex.

A recombination template may be a component of another vector as described herein, contained in a separate vector, or provided as a separate polynucleotide. A recombination template polynucleotide may be of any suitable length, such as about or more than about 10, 15, 20, 25, 50, 75, 100, 150, 200, 500, 1000, or more nucleotides in length. In one embodiment, the recombination template polynucleotide is complementary to a portion of a polynucleotide comprising the target sequence. When optimally aligned, a recombination template polynucleotide might overlap with one or more nucleotides of a target sequences (e.g., about or more than about 1, 5, 10, 15, 20, 25, 30, 35, 40, 45, 50, 60, 70, 80, 90, 100 or more nucleotides). In one embodiment, when a recombination template sequence and a polynucleotide comprising a target sequence are optimally aligned, the nearest nucleotide of the recombination template polynucleotide is within about 1, 5, 10, 15, 20, 25, 50, 75, 100, 200, 300, 400, 500, 1000, 5000, 10000, or more nucleotides from the target sequence.

In an embodiment, the recombination template nucleic acid alters the structure of the target position by participating in homologous recombination. In an embodiment, the recombination template nucleic acid alters the sequence of the target position. In an embodiment, the recombination template nucleic acid results in the incorporation of a modified, or non-naturally occurring base into the target nucleic acid.

The recombination template sequence may undergo a breakage mediated or catalyzed recombination with the target sequence. In an embodiment, the recombination template nucleic acid may include sequence that corresponds to a site on the target sequence that is cleaved by an Cas5-HNH or Cas8-HNH polypeptide nuclease mediated cleavage event. In an embodiment, the recombination template nucleic acid may include sequence that corresponds to both, a first site on the target sequence that is cleaved in a first Cas5-HNH or Cas8-HNH polypeptide nuclease mediated event and a second site on the target sequence that is cleaved in a second Cas5-HNH or Cas8-HNH polypeptide nuclease mediated event.

In an embodiment, the recombination template nucleic acid can include sequence which results in an alteration in the coding sequence of a translated sequence, e.g., one which results in the substitution of one amino acid for another in a protein product, e.g., transforming a mutant allele into a wild-type allele, transforming a wild-type allele into a mutant allele, and/or introducing a stop codon, insertion of an amino acid residue, deletion of an amino acid residue, or a nonsense mutation. In an embodiment, the recombination template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A recombination template nucleic acid having homology with a target position in a target gene may be used to alter the structure of a target sequence. The recombination template sequence may be used to alter an unwanted structure, e.g., an unwanted or mutant nucleotide. The recombination template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The recombination template nucleic acid may include sequence which results in: a change in sequence of 1, 2, 3, 4, 5, 6, 7, 8, 9, 10, 11, 12 or more nucleotides of the target sequence. In an embodiment, the recombination template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, or 220+/−10 nucleotides in length. In an embodiment, the recombination template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, or 220+/−20 nucleotides in length. In an embodiment, the recombination template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

A recombination template nucleic acid comprises the following components: [5′ homology arm]-[replacement sequence]-[3′ homology arm]. The homology arms provide for recombination into the chromosome, thus replacing the undesired element, e.g., a mutation or signature, with the replacement sequence. In an embodiment, the homology arms flank the most distal cleavage sites. In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In an embodiment, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In one embodiment, both the 5′ and the 3′ homology arms may be shortened to avoid including certain sequence repeat elements.

In an embodiment, a recombination template nucleic acids for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Mutating key residues in the DNA cleavage domains of the Cas5-HNH or Cas8-HNH polypeptide nuclease, results in the generation of a catalytically inactive Cas5-HNH or Cas8-HNH polypeptide nuclease. A catalytically inactive Cas5-HNH or Cas8-HNH polypeptide complexes with a guide sequence RNA and localizes to the DNA sequence specified by that guide RNA's targeting domain, however, it does not cleave the target DNA. Fusion of the inactive Cas5-HNH or Cas8-HNH polypeptide to an effector domain, e.g., a transcription repression domain, enables recruitment of the effector to any DNA site specified by the guide RNA. In an embodiment, Cas5-HNH or Cas8-HNH polypeptide may be fused to a transcriptional repression domain and recruited to the promoter region of a gene. Especially for gene repression, it is contemplated herein that blocking the binding site of an endogenous transcription factor would aid in downregulating gene expression. In another embodiment, an inactive Cas5-HNH or Cas8-HNH polypeptide can be fused to a chromatin modifying protein. Altering chromatin status can result in decreased expression of the target gene.

In an embodiment, a guide sequence RNA can be targeted to known transcription response elements (e.g., promoters, enhancers, etc.), known upstream activating sequences, and/or sequences of unknown or known function that are suspected of being able to control expression of the target DNA.

In some methods, a target polynucleotide can be inactivated to affect the modification of the expression in a cell. For example, upon the binding of a composition to a target sequence in a cell, the target polynucleotide is inactivated such that the sequence is not transcribed, the coded protein is not produced, or the sequence does not function as the wild-type sequence does. For example, a protein or microRNA coding sequence may be inactivated such that the protein is not produced.

### Non-Homologous End-Joining

In an embodiment, nuclease-induced non-homologous end-joining (NHEJ) can be used to target gene-specific knockouts. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. Two-thirds of these mutations typically alter the reading frame and, therefore, produce a non-functional protein. Additionally, mutations that maintain the reading frame, but which insert or delete a significant amount of sequence, can destroy functionality of the protein. This is locus dependent as mutations in critical functional domains are likely less tolerable than mutations in non-critical regions of the protein. The indel mutations generated by NHEJ are unpredictable in nature; however, at a given break site certain indel sequences are favored and are over-represented in the population, likely due to small regions of microhomology. The lengths of deletions can vary widely; most commonly in the 1-50 bp range, but they can easily be greater than 50 bp, e.g., they can easily reach greater than about 100-200 bp. Insertions tend to be shorter and often include short duplications of the sequence immediately surrounding the break site. However, it is possible to obtain large insertions, and in these cases, the inserted sequence has often been traced to other regions of the genome or to plasmid DNA present in the cells.

Because NHEJ is a mutagenic process, it may also be used to delete small sequence motifs as long as the generation of a specific final sequence is not required. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences; however, the error-prone nature of NHEJ may still produce indel mutations at the site of repair.

Both double strand cleaving Cas5-HNH or Cas8-HNH polypeptide nuclease, or an ortholog or homolog thereof, and single strand, or nickase, Cas5-HNH or Cas8-HNH polypeptide nuclease, or an ortholog or homolog thereof, molecules can be used in the methods and compositions described herein to generate NHEJ-mediated indels. NHEJ-mediated indels targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp).

In an embodiment, in which a guide RNA and Cas5-HNH or Cas8-HNH polypeptide nuclease, or an ortholog or homolog thereof, generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position).

In an embodiment, in which two guide RNAs complexing with Cas5-HNH or Cas8-HNH polypeptide nuclease, or an ortholog or homolog thereof, e.g., Cas5-HNH or Cas8-HNH polypeptide nickases induce two single strand breaks for the purpose of inducing NHEJ-mediated indels, two guide RNAs may be configured to position two single-strand breaks to provide for NHEJ repair a nucleotide of the target position.

In some examples, the systems herein may introduce one or more indels via NHEJ pathway and insert sequence from a combination template via HDR.

### Exemplary Applications

The invention provides a non-naturally occurring or engineered composition, or one or more polynucleotides encoding components of said composition, or vector or delivery systems comprising one or more polynucleotides encoding components of said composition for use in a modifying a target cell in vivo, ex vivo or in vitro and, may be conducted in a manner alters the cell such that once modified the progeny or cell line of the Cas5-HNH or Cas8-HNH polypeptide or CRISPR-associated Cas5-HNH or Cas8-HNH polypeptide nuclease modified cell retains the altered phenotype. The modified cells and progeny may be part of a multi-cellular organism such as a plant or animal with ex vivo or in vivo application of composition to desired cell types. The methods herein include a therapeutic method of treatment. The therapeutic method of treatment may comprise gene or genome editing, or gene therapy.

In one embodiment, one or more vectors described herein are used to produce a non-human transgenic animal or transgenic plant. In one embodiment, the transgenic animal is a mammal, such as a mouse, rat, or rabbit. Methods for producing transgenic animals and plants are known in the art, and generally begin with a method of cell transfection, such as described herein. Detection methods such as FISH

In one aspect, the invention provides an engineered, non-naturally occurring composition comprising a catalytically inactivate Cas5-HNH or Cas8-HNH polypeptide nuclease described herein and use this system in detection methods such as fluorescence in situ hybridization (FISH). A dead Cas5-HNH or Cas8-HNH polypeptide nuclease which lacks the ability to produce DNA double-strand breaks may be fused with a marker, such as fluorescent protein, such as the enhanced green fluorescent protein (eEGFP) and co-expressed with small guide RNAs to target pericentric, centric and teleomeric repeats in vivo. The dead Cas5-HNH or Cas8-HNH polypeptide (see, e.g., Table 1E) or CRISPR-associated Cas5-HNH or Cas8-HNH polypeptide nuclease system can be used to visualize both repetitive sequences and individual genes in the human genome. Such new applications of labelled dead Cas5-HNH or Cas8-HNH polypeptide nuclease may be important in imaging cells and studying the functional nuclear architecture, especially in cases with a small nucleus volume or complex 3-D structures. (Chen B, Gilbert L A, Cimini B A, Schnitzbauer J, Zhang W, Li G W, Park J, Blackburn E H, Weissman J S, Qi L S, Huang B. 2013. Dynamic imaging of genomic loci in living human cells by an optimized CRISPR/Cas system. Cell 155 (7): 1479-91. doi: 10.1016/j.cell.2013.12.001.)

### Patient-Specific Screening Methods

A nucleic acid-targeting system that targets DNA, e.g., trinucleotide repeats can be used to screen patients or patent samples for the presence of such repeats. The repeats can be the target of the RNA of the nucleic acid-targeting system, and if there is binding thereto by the nucleic acid-targeting system, that binding can be detected, to thereby indicate that such a repeat is present. Thus, a nucleic acid-targeting system can be used to screen patients or patient samples for the presence of the repeat. The patient can then be administered suitable compound(s) to address the condition or can be administered a nucleic acid-targeting system to bind to and cause insertion, deletion or mutation and alleviate the condition.

### Models of Genetic and Epigenetic Conditions

A method of the invention may be used to create a plant, an animal or cell that may be used to model and/or study genetic or epigenetic conditions of interest, such as a through a model of mutations of interest or a disease model. As used herein, “disease” refers to a disease, disorder, or indication in a subject. For example, a method of the invention may be used to create an animal or cell that comprises a modification in one or more nucleic acid sequences associated with a disease, or a plant, animal or cell in which the expression of one or more nucleic acid sequences associated with a disease are altered. Such a nucleic acid sequence may encode a disease associated protein sequence or may be a disease associated control sequence. Accordingly, it is understood that in embodiments of the invention, a plant, subject, patient, organism or cell can be a non-human subject, patient, organism or cell. Thus, the invention provides a plant, animal or cell, produced by the present methods, or a progeny thereof. The progeny may be a clone of the produced plant or animal or may result from sexual reproduction by crossing with other individuals of the same species to introgress further desirable traits into their offspring. The cell may be in vivo or ex vivo in the cases of multicellular organisms, particularly animals or plants. In the instance where the cell is in cultured, a cell line may be established if appropriate culturing conditions are met and preferably if the cell is suitably adapted for this purpose (for instance a stem cell). Bacterial cell lines produced by the invention are also envisaged. Hence, cell lines are also envisaged.

In some methods, the disease model can be used to study the effects of mutations on the animal or cell and development and/or progression of the disease using measures commonly used in the study of the disease. Alternatively, such a disease model is useful for studying the effect of a pharmaceutically active compound on the disease.

In some methods, the disease model can be used to assess the efficacy of a potential gene therapy strategy. That is, a disease-associated gene or polynucleotide can be modified such that the disease development and/or progression is inhibited or reduced. In particular, the method comprises modifying a disease-associated gene or polynucleotide such that an altered protein is produced and, as a result, the animal or cell has an altered response. Accordingly, in some methods, a genetically modified animal may be compared with an animal predisposed to development of the disease such that the effect of the gene therapy event may be assessed.

In another embodiment, this invention provides a method of developing a biologically active agent that modulates a cell signaling event associated with a disease gene. The method comprises contacting a test compound with a cell comprising one or more vectors that drive expression of one or more of a Cas5-HNH or Cas8-HNH polypeptide nuclease, and a conserved nucleotide sequence linked to a guide sequence; and detecting a change in a readout that is indicative of a reduction or an augmentation of a cell signaling event associated with, e.g., a mutation in a disease gene contained in the cell.

A cell model or animal model can be constructed in combination with the method of the invention for screening a cellular function change. Such a model may be used to study the effects of a genome sequence modified by the complex of the invention on a cellular function of interest. For example, a cellular function model may be used to study the effect of a modified genome sequence on intracellular signaling or extracellular signaling. Alternatively, a cellular function model may be used to study the effects of a modified genome sequence on sensory perception. In some such models, one or more genome sequences associated with a signaling biochemical pathway in the model are modified.

Several disease models have been specifically investigated. These include de novo autism risk genes CHD8, KATNAL2, and SCN2A; and the syndromic autism (Angelman Syndrome) gene UBE3A. These genes, and resulting autism models are of course preferred, but serve to show the broad applicability of the invention across genes and corresponding models. An altered expression of one or more genome sequences associated with a signaling biochemical pathway can be determined by assaying for a difference in the mRNA levels of the corresponding genes between the test model cell and a control cell, when they are contacted with a candidate agent. Alternatively, the differential expression of the sequences associated with a signaling biochemical pathway is determined by detecting a difference in the level of the encoded polypeptide or gene product.

To assay for an agent-induced alteration in the level of mRNA transcripts or corresponding polynucleotides, nucleic acid contained in a sample is first extracted according to standard methods in the art. For instance, mRNA can be isolated using various lytic enzymes or chemical solutions according to the procedures set forth in Sambrook et al. (1989) or extracted by nucleic-acid-binding resins following the accompanying instructions provided by the manufacturers. The mRNA contained in the extracted nucleic acid sample is then detected by amplification procedures or conventional hybridization assays (e.g., Northern blot analysis) according to methods widely known in the art or based on the methods exemplified herein.

For purpose of this invention, amplification means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. In particular, the isolated RNA can be subjected to a reverse transcription assay that is coupled with a quantitative polymerase chain reaction (RT-PCR) in order to quantify the expression level of a sequence associated with a signaling biochemical pathway.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

In yet another aspect, conventional hybridization assays using hybridization probes that share sequence homology with sequences associated with a signaling biochemical pathway can be performed. Typically, probes are allowed to form stable complexes with the sequences associated with a signaling biochemical pathway contained within the biological sample derived from the test subject in a hybridization reaction. It will be appreciated by one of skill in the art that where antisense is used as the probe nucleic acid, the target polynucleotides provided in the sample are chosen to be complementary to sequences of the antisense nucleic acids. Conversely, where the nucleotide probe is a sense nucleic acid, the target polynucleotide is selected to be complementary to sequences of the sense nucleic acid.

Hybridization can be performed under conditions of various stringency. Suitable hybridization conditions for the practice of the present invention are such that the recognition interaction between the probe and sequences associated with a signaling biochemical pathway is both sufficiently specific and sufficiently stable. Conditions that increase the stringency of a hybridization reaction are widely known and published in the art. See, for example, (Sambrook, et al., (1989); Nonradioactive In Situ Hybridization Application Manual, Boehringer Mannheim, second edition). The hybridization assay can be formed using probes immobilized on any solid support, including but are not limited to nitrocellulose, glass, silicon, and a variety of gene arrays. A preferred hybridization assay is conducted on high-density gene chips as described in U.S. Pat. No. 5,445,934.

For a convenient detection of the probe-target complexes formed during the hybridization assay, the nucleotide probes are conjugated to a detectable label. Detectable labels suitable for use in the present invention include any composition detectable by photochemical, biochemical, spectroscopic, immunochemical, electrical, optical or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, β-galactosidase, urease, alkaline phosphatase or peroxidase, avidin/biotin complex.

The detection methods used to detect or quantify the hybridization intensity will typically depend upon the label selected above. For example, radiolabels may be detected using photographic film or a phosphoimager. Fluorescent markers may be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

An agent-induced change in expression of sequences associated with a signaling biochemical pathway can also be determined by examining the corresponding gene products. Determining the protein level typically involves a) contacting the protein contained in a biological sample with an agent that specifically bind to a protein associated with a signaling biochemical pathway; and (b) identifying any agent: protein complex so formed. In one aspect of this embodiment, the agent that specifically binds a protein associated with a signaling biochemical pathway is an antibody, preferably a monoclonal antibody.

The reaction is performed by contacting the agent with a sample of the proteins associated with a signaling biochemical pathway derived from the test samples under conditions that will allow a complex to form between the agent and the proteins associated with a signaling biochemical pathway. The formation of the complex can be detected directly or indirectly according to standard procedures in the art. In the direct detection method, the agents are supplied with a detectable label and unreacted agents may be removed from the complex; the amount of remaining label thereby indicating the amount of complex formed. For such method, it is preferable to select labels that remain attached to the agents even during stringent washing conditions. It is preferable that the label does not interfere with the binding reaction. In the alternative, an indirect detection procedure may use an agent that contains a label introduced either chemically or enzymatically. A desirable label generally does not interfere with binding or the stability of the resulting agent:polypeptide complex. However, the label is typically designed to be accessible to an antibody for an effective binding and, hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent:polypeptide complexes formed during the binding reaction can be quantified by standard quantitative assays. As illustrated above, the formation of agent:polypeptide complex can be measured directly by the amount of label remained at the site of binding. In an alternative, the protein associated with a signaling biochemical pathway is tested for its ability to compete with a labeled analog for binding sites on the specific agent. In this competitive assay, the amount of label captured is inversely proportional to the amount of protein sequences associated with a signaling biochemical pathway present in a test sample.

A number of techniques for protein analysis based on the general principles outlined above are available in the art. They include but are not limited to radioimmunoassay, ELISA (enzyme linked immunoradiometric assays), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Antibodies that specifically recognize or bind to proteins associated with a signaling biochemical pathway are preferable for conducting the aforementioned protein analyses. Where desired, antibodies that recognize a specific type of post-translational modifications (e.g., signaling biochemical pathway inducible modifications) can be used. Post-translational modifications include but are not limited to glycosylation, lipidation, acetylation, and phosphorylation. These antibodies may be purchased from commercial vendors. For example, anti-phosphotyrosine antibodies that specifically recognize tyrosine-phosphorylated proteins are available from a number of vendors including Invitrogen and Perkin Elmer. Anti-phosphotyrosine antibodies are particularly useful in detecting proteins that are differentially phosphorylated on their tyrosine residues in response to an ER stress. Such proteins include but are not limited to eukaryotic translation initiation factor 2 alpha (eIF-2a). Alternatively, these antibodies can be generated using conventional polyclonal or monoclonal antibody technologies by immunizing a host animal or an antibody-producing cell with a target protein that exhibits the desired post-translational modification.

### Genome Wide Knock-out Screening

The Cas5-HNH or Cas8-HNH polypeptide nuclease and systems described herein can be used to perform efficient and cost effective functional genomic screens. Such screens can utilize Cas5-HNH or Cas8-HNH polypeptide nuclease-based genome wide libraries. Such screens and libraries can provide for determining the function of genes, cellular pathways genes are involved in, and how any alteration in gene expression can result in a particular biological process. An advantage of the present invention is that the composition avoids off-target binding and its resulting side effects. This is achieved using systems arranged to have a high degree of sequence specificity for the target DNA. In preferred embodiments of the invention, the Cas5-HNH or Cas8-HNH polypeptide nuclease complexes are Cas5-HNH or Cas8-HNH polypeptide nuclease complexes.

In embodiments of the invention, a genome wide library may comprise a plurality of Cas5-HNH or Cas8-HNH polypeptide nuclease guide RNAs, as described herein, comprising guide sequences that are capable of targeting a plurality of target sequences in a plurality of genomic loci in a population of eukaryotic cells. The population of cells may be a population of embryonic stem (ES) cells. The target sequence in the genomic locus may be a non-coding sequence. The non-coding sequence may be an intron, regulatory sequence, splice site, 3′ UTR, 5′ UTR, or polyadenylation signal. Gene function of one or more gene products may be altered by said targeting. The targeting may result in a knockout of gene function. The targeting of a gene product may comprise more than one guide RNA. A gene product may be targeted by 2, 3, 4, 5, 6, 7, 8, 9, or 10 guide RNAs, preferably 3 to 4 per gene. Off-target modifications may be minimized by exploiting the staggered double strand breaks generated by Cas5-HNH or Cas8-HNH polypeptide nuclease complexes or by utilizing methods analogous to those used in composition (See, e.g., DNA targeting specificity of RNA-guided Cas nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi: 10.1038/nbt.2647 (2013)), incorporated herein by reference. The targeting may be of about 100 or more sequences. The targeting may be of about 1000 or more sequences. The targeting may be of about 20,000 or more sequences. The targeting may be of the entire genome. The targeting may be of a panel of target sequences focused on a relevant or desirable pathway. The pathway may be an immune pathway. The pathway may be a cell division pathway.

One aspect of the invention comprehends a genome wide library that may comprise a plurality of guide RNAs that may comprise guide sequences that are capable of targeting a plurality of target sequences in a plurality of genomic loci, wherein said targeting results in a knockout of gene function. This library may potentially comprise guide RNAs that target each and every gene in the genome of an organism.

In one embodiment of the invention, the organism or subject is a eukaryote (including mammal including human) or a non-human eukaryote or a non-human animal or a non-human mammal. In one embodiment, the organism or subject is a non-human animal, and may be an arthropod, for example, an insect, or may be a nematode. In some methods of the invention the organism or subject is a plant. In some methods of the invention, the organism or subject is a mammal or a non-human mammal. A non-human mammal may be for example a rodent (preferably a mouse or a rat), an ungulate, or a primate. In some methods of the invention the organism or subject is algae, including microalgae, or is a fungus.

The knockout of gene function may comprise introducing into each cell in the population of cells a vector system of one or more vectors comprising an engineered, non-naturally occurring composition herein. The guide sequence may target a unique gene in each cell, wherein the Cas5-HNH or Cas8-HNH polypeptide nuclease is operably linked to a regulatory element, wherein when transcribed, the guide RNA comprising the guide sequence directs sequence-specific binding of the Cas5-HNH or Cas8-HNH polypeptide nuclease to a target sequence in the genomic loci of the unique gene, inducing cleavage of the genomic loci by the Cas5-HNH or Cas8-HNH polypeptide nuclease, and confirming different knockout mutations in a plurality of unique genes in each cell of the population of cells thereby generating a gene knockout cell library. The invention comprehends that the population of cells is a population of eukaryotic cells, and in a preferred embodiment, the population of cells is a population of embryonic stem (ES) cells.

The one or more vectors may be plasmid vectors. The vector may be a single vector comprising a Cas5-HNH or Cas8-HNH polypeptide nuclease, a guide RNA, and optionally, a selection marker into target cells. Not being bound by a theory, the ability to simultaneously deliver a Cas5-HNH or Cas8-HNH polypeptide nuclease and hRNA through a single vector enables application to any cell type of interest, without the need to first generate cell lines that express the Cas5-HNH or Cas8-HNH polypeptide nuclease. The regulatory element may be an inducible promoter. The inducible promoter may be a doxycycline inducible promoter. In some methods of the invention the expression of the guide sequence is under the control of the T7 promoter and is driven by the expression of T7 polymerase. The confirming of different knockout mutations may be by whole exome sequencing. The knockout mutation may be achieved in 100 or more unique genes. The knockout mutation may be achieved in 1000 or more unique genes. The knockout mutation may be achieved in 20,000 or more unique genes. The knockout mutation may be achieved in the entire genome. The knockout of gene function may be achieved in a plurality of unique genes which function in a particular physiological pathway or condition. The pathway or condition may be an immune pathway or condition. The pathway or condition may be a cell division pathway or condition.

Useful in the practice of the instant invention utilizing Cas5-HNH or Cas8-HNH polypeptide nuclease complexes are methods used in compositions and reference is made to: Genome-Scale CRISPR-Cas Knockout Screening in Human Cells. Shalem, O., Sanjana, NE., Hartenian, E., Shi, X., Scott, DA., Mikkelson, T., Heckl, D., Ebert, BL., Root, DE., Doench, JG., Zhang, F. Science December 12. (2013). [Epub ahead of print]; Published in final edited form as: Science. 2014 Jan. 3; 343 (6166): 84-87. Shalem et al. involves a new way to interrogate gene function on a genome-wide scale. Their studies showed that delivery of a genome-scale CRISPR-Cas knockout (GeCKO) library targeted 18,080 genes with 64,751 unique guide sequences enabled both negative and positive selection screening in human cells. First, the authors showed use of the GeCKO library to identify genes essential for cell viability in cancer and pluripotent stem cells. Next, in a melanoma model, the authors screened for genes whose loss is involved in resistance to vemurafenib, a therapeutic that inhibits mutant protein kinase BRAF. Their studies showed that the highest-ranking candidates included previously validated genes NF1 and MED12 as well as novel hitsNF2, CUL3, TADA2B, and TADA1. The authors observed a high level of consistency between independent guide RNAs targeting the same gene and a high rate of hit confirmation, and thus demonstrated the promise of genome-scale screening with Cas5-HNH or Cas8-HNH polypeptide nuclease.

Reference is also made to US patent publication number US20140357530; and PCT Patent Publication WO2014093701, hereby incorporated herein by reference. Reference is also made to NIH Press Release of Oct. 22, 2015 entitled, “Researchers identify potential alternative to CRISPR-Cas genome editing tools: New Cas enzymes shed light on evolution of CRISPR-Cas systems, which is incorporated by reference.

### Functional Alteration and Screening

In another aspect, the present invention provides for a method of functional evaluation and screening of genes. The use of the compositions to precisely deliver functional domains, to activate or repress genes or to alter epigenetic state by precisely altering the methylation site on a specific locus of interest, can be with one or more guide RNAs applied to a single cell or population of cells or with a library applied to genome in a pool of cells ex vivo or in vivo comprising the administration or expression of a library comprising a plurality of hRNAs (comprising guide molecules) and wherein the screening further comprises use of a Cas5-HNH or Cas8-HNH polypeptide nuclease, wherein the complex comprising the Cas5-HNH or Cas8-HNH polypeptide nuclease is modified to comprise a heterologous functional domain. In an aspect the invention provides a method for screening a genome comprising the administration to a host or expression in a host in vivo of a library. In an aspect the invention provides a method as herein discussed further comprising an activator administered to the host or expressed in the host. In an aspect the invention provides a method as herein discussed wherein the activator is attached to a Cas5-HNH or Cas8-HNH polypeptide nuclease. In an aspect the invention provides a method as herein discussed wherein the activator is attached to the N terminus or the C terminus of the Cas5-HNH or Cas8-HNH polypeptide nuclease. In an aspect the invention provides a method as herein discussed wherein the activator is attached to a guide RNA loop. In an aspect the invention provides a method as herein discussed further comprising a repressor administered to the host or expressed in the host. In an aspect the invention provides a method as herein discussed, wherein the screening comprises affecting and detecting gene activation, gene inhibition, or cleavage in the locus.

It is also preferred to target endogenous (regulatory) control elements (such as enhancers and silencers) e.g., in addition to a promoter or promoter-proximal elements. Thus, the invention can also be used to target endogenous control elements (including enhancers and silencers) in addition to targeting of the promoter. These control elements can be located upstream and downstream of the transcriptional start site (TSS), starting from 200 bp from the TSS to 100 kb away. Targeting of known control elements can be used to activate or repress the gene of interest. In some cases, a single control element can influence the transcription of multiple target genes. Targeting of a single control element could therefore be used to control the transcription of multiple genes simultaneously.

Targeting of putative control elements on the other hand (e.g., by tiling the region of the putative control element as well as 200 bp up to 100 kB around the element) can be used as a means to verify such elements (by measuring the transcription of the gene of interest) or to detect novel control elements (e.g., by tiling 100 kb upstream and downstream of the TSS of the gene of interest). In addition, targeting of putative control elements can be useful in the context of understanding genetic causes of disease. Many mutations and common SNP variants associated with disease phenotypes are located outside coding regions. Targeting of such regions with either the activation or repression systems described herein can be followed by readout of transcription of either a) a set of putative targets (e.g., a set of genes located in closest proximity to the control element) or b) whole-transcriptome readout by e.g., RNA-seq or microarray. This would allow for the identification of likely candidate genes involved in the disease phenotype. Such candidate genes could be useful as novel drug targets.

Histone acetyltransferase (HAT) inhibitors are mentioned herein. However, an alternative, in one embodiment is for the one or more functional domains to comprise an acetyltransferase, preferably a histone acetyltransferase. These are useful in the field of epigenomics, for example in methods of interrogating the epigenome. Methods of interrogating the epigenome may include, for example, targeting epigenomic sequences. Targeting epigenomic sequences may include the guide being directed to an epigenomic target sequence. Epigenomic target sequence may include, in one embodiment, include a promoter, silencer or an enhancer sequence.

### Saturating Mutagenesis

The compositions herein can be used to perform saturating or deep scanning mutagenesis of genomic loci in conjunction with a cellular phenotype—for instance, for determining critical minimal features and discrete vulnerabilities of functional elements required for gene expression, drug resistance, and reversal of disease. By saturating or deep scanning mutagenesis is meant that every or essentially every DNA base is cut within the genomic loci. A library of Cas1 effector protein guide RNAs may be introduced into a population of cells. The library may be introduced, such that each cell receives a single hRNA. In the case where the library is introduced by transduction of a viral vector, as described herein, a low multiplicity of infection (MOI) is used. The library may include guide RNAs targeting every sequence upstream of a PAM sequence in a genomic locus. The library may include at least 100 non-overlapping genomic sequences upstream of a PAM sequence for every 1000 base pairs within the genomic locus. The library may include hRNAs targeting sequences upstream of at least one different PAM sequence. The composition may include more than one Cas5-HNH or Cas8-HNH polypeptide nuclease. Any Cas5-HNH or Cas8-HNH polypeptide nuclease protein as described herein, including orthologues or engineered Cas5-HNH or Cas8-HNH polypeptide nuclease that recognize different PAM sequences may be used. The frequency of off target sites for a guide RNA may be less than 500. Off target scores may be generated to select guide RNAs with the lowest off target sites. Any phenotype determined to be associated with cutting at a hRNA target site may be confirmed by using guide RNAs targeting the same site in a single experiment. Validation of a target site may also be performed by using a modified Cas5-HNH or Cas8-HNH polypeptide nuclease, as described herein, and two hRNAs targeting the genomic site of interest. Not being bound by a theory, a target site is a true hit if the change in phenotype is observed in validation experiments.

The genomic loci may include at least one continuous genomic region. The at least one continuous genomic region may comprise up to the entire genome. The at least one continuous genomic region may comprise a functional element of the genome. The functional element may be within a non-coding region, coding gene, intronic region, promoter, or enhancer. The at least one continuous genomic region may comprise at least 1 kb, preferably at least 50 kb of genomic DNA. The at least one continuous genomic region may comprise a transcription factor binding site. The at least one continuous genomic region may comprise a region of DNase I hypersensitivity. The at least one continuous genomic region may comprise a transcription enhancer or repressor element. The at least one continuous genomic region may comprise a site enriched for an epigenetic signature. The at least one continuous genomic DNA region may comprise an epigenetic insulator. The at least one continuous genomic region may comprise two or more continuous genomic regions that physically interact. Genomic regions that interact may be determined by ‘4C technology’. 4C technology allows the screening of the entire genome in an unbiased manner for DNA segments that physically interact with a DNA fragment of choice, as is described in Zhao et al. ((2006) Nat Genet 38, 1341-7) and in U.S. Pat. No. 8,642,295, both incorporated herein by reference in its entirety. The epigenetic signature may be histone acetylation, histone methylation, histone ubiquitination, histone phosphorylation, DNA methylation, or a lack thereof.

The compositions for saturating or deep scanning mutagenesis can be used in a population of cells. The compositions can be used in eukaryotic cells, including but not limited to mammalian and plant cells. The population of cells may be prokaryotic cells. The population of eukaryotic cells may be a population of embryonic stem (ES) cells, neuronal cells, epithelial cells, immune cells, endocrine cells, muscle cells, erythrocytes, lymphocytes, plant cells, or yeast cells.

In one aspect, the present invention provides for a method of screening for functional elements associated with a change in a phenotype. The library may be introduced into a population of cells that are adapted to contain a Cas5-HNH or Cas8-HNH polypeptide nuclease. The cells may be sorted into at least two groups based on the phenotype. The phenotype may be expression of a gene, cell growth, or cell viability. The relative representation of the guide RNAs present in each group are determined, whereby genomic sites associated with the change in phenotype are determined by the representation of guide RNAs present in each group. The change in phenotype may be a change in expression of a gene of interest. The gene of interest may be upregulated, downregulated, or knocked out. The cells may be sorted into a high expression group and a low expression group. The population of cells may include a reporter construct that is used to determine the phenotype. The reporter construct may include a detectable marker. Cells may be sorted by use of the detectable marker.

In another aspect, the present invention provides for a method of screening for genomic sites associated with resistance to a chemical compound. The chemical compound may be a drug or pesticide. The library may be introduced into a population of cells that are adapted to contain a Cas5-HNH or Cas8-HNH polypeptide nuclease, wherein each cell of the population contains no more than one guide RNA; the population of cells are treated with the chemical compound; and the representation of guide RNAs are determined after treatment with the chemical compound at a later time point as compared to an early time point, whereby genomic sites associated with resistance to the chemical compound are determined by enrichment of guide RNAs. Representation of guide RNAs may be determined by deep sequencing methods.

Useful in the practice of the instant invention utilizing compositions are methods used in compositions and reference is made to the article entitled BCL11A enhancer dissection by Cas-mediated in situ saturating mutagenesis. Canver, M. C., Smith, E. C., Sher, F., Pinello, L., Sanjana, N. E., Shalem, O., Chen, D. D., Schupp, P. G., Vinjamur, D. S., Garcia, S. P., Luc, S., Kurita, R., Nakamura, Y., Fujiwara, Y., Maeda, T., Yuan, G., Zhang, F., Orkin, S. H., & Bauer, D. E. DOI: 10.1038/nature15521, published online Sep. 16, 2015, the article is herein incorporated by reference and discussed briefly below:

Canver et al. involves novel pooled guide RNA libraries to perform in situ saturating mutagenesis of the human and mouse BCL11A erythroid enhancers previously identified as an enhancer associated with fetal hemoglobin (HbF) level and whose mouse ortholog is necessary for erythroid BCL11A expression. This approach revealed critical minimal features and discrete vulnerabilities of these enhancers. Through editing of primary human progenitors and mouse transgenesis, the authors validated the BCL11A erythroid enhancer as a target for HbF reinduction. The authors generated a detailed enhancer map that informs therapeutic genome editing.

### Modification of a Cell or Organism

The present disclosure further provides cells comprising one or more components of the systems herein, e.g., the Cas5-HNH or Cas8-HNH polypeptide nuclease and/or guide RNA(s). Also provided include cells modified by the systems and methods herein, and cell cultures, tissues, organs, organism comprising such cells or progeny thereof. The invention, in one embodiment, comprehends a method of modifying a cell or organism. The cell may be a prokaryotic cell or a eukaryotic cell. The cell may be a mammalian cell. The mammalian cell many be a non-human primate, bovine, porcine, rodent or mouse cell. The cell may be a non-mammalian eukaryotic cell such as poultry, fish or shrimp. The cell may also be a plant cell. The plant cell may be of a crop plant such as cassava, corn, sorghum, wheat, or rice. The plant cell may also be of an algae, tree or vegetable. The modification introduced to the cell by the present invention may be such that the cell and progeny of the cell are altered for improved production of biologic products such as an antibody, starch, alcohol or other desired cellular output. The modification introduced to the cell by the present invention may be such that the cell and progeny of the cell include an alteration that changes the biologic product produced.

### Therapeutic Uses and Methods of Treatment

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a polynucleotide in a subject or cell thereof using a composition, system, or component thereof described herein and/or include detecting a diseased or healthy polynucleotide in a subject or cell thereof using a composition, system, or component thereof described herein. In one embodiment, the method of treatment or prevention can include using a composition, system, or component thereof to modify a polynucleotide of an infectious organism (e.g., bacterial or virus) within a subject or cell thereof. In one embodiment, the method of treatment or prevention can include using a composition, system, or component thereof to modify a polynucleotide of an infectious organism or symbiotic organism within a subject. The composition, system, and components thereof can be used to develop models of diseases, states, or conditions. The composition, system, and components thereof can be used to detect a disease state or correction thereof, such as by a method of treatment or prevention described herein. The composition, system, and components thereof can be used to screen and select cells that can be used, for example, as treatments or preventions described herein. The composition, system, and components thereof can be used to develop biologically active agents that can be used to modify one or more biologic functions or activities in a subject or a cell thereof.

In general, the method can include delivering a composition, system, and/or component thereof to a subject or cell thereof, or to an infectious or symbiotic organism by a suitable delivery technique and/or composition. Once administered the components can operate as described elsewhere herein to elicit a nucleic acid modification event. In some aspects, the nucleic acid modification event can occur at the genomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

The composition, system, and components thereof described elsewhere herein can be used to treat and/or prevent a disease, such as a genetic and/or epigenetic disease, in a subject. The composition, system, and components thereof described elsewhere herein can be used to treat and/or prevent genetic infectious diseases in a subject, such as bacterial infections, viral infections, fungal infections, parasite infections, and combinations thereof. The composition, system, and components thereof described elsewhere herein can be used to modify the composition or profile of a microbiome in a subject, which can in turn modify the health status of the subject. The composition, system, described herein can be used to modify cells ex vivo, which can then be administered to the subject whereby the modified cells can treat or prevent a disease or symptom thereof. This is also referred to in some contexts as adoptive therapy. The composition, system, described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the composition, system, or complex or any of polynucleotides or vectors described herein and administering them to the subject. A suitable repair template may also be provided, for example delivered by a vector comprising said repair template. The repair template may be a recombination template herein. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of composition, system, complex or component thereof comprising multiple Cas5-HNH or Cas8-HINH polypeptide nucleases. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with the Cas5-HNH or Cas8-HNH polypeptide nuclease(s), advantageously encoding and expressing in vivo the remaining portions of the composition, system, (e.g., RNA, guides). A suitable repair template may also be provided, for example delivered by a vector comprising said repair template. Also provided is a method of treating a subject, e.g., a subject in need thereof, comprising inducing transcriptional activation or repression by transforming the subject with the Cas5-HNH or Cas8-HNH polypeptide nuclease(s) advantageously encoding and expressing in vivo the remaining portions of the composition, system, (e.g., RNA, guides); advantageously In one embodiment the Cas5-HNH or Cas8-HNH polypeptide nuclease is a catalytically inactive Cas5-HNH or Cas8-HNH polypeptide nuclease and includes one or more associated functional domains. Where any treatment is occurring ex vivo, for example in a cell culture, then it will be appreciated that the term ‘subject’ may be replaced by the phrase “cell or cell culture.”

One or more components of the composition and system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral hRNA selection) and concentration of guide RNA (e.g., dependent on whether multiple guide RNAs are used) may be advantageous for eliciting an improved effect.

Thus, also described herein are methods of inducing one or more polynucleotide modifications in a eukaryotic or prokaryotic cell or component thereof (e.g., a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo.

In one embodiment, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiment or the cell of any one of the above embodiment.

Also provided herein is the use of the particle delivery system or the delivery system or the virus particle of any one of the above embodiments or the cell of any one of the above embodiments in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism.

In one embodiment, polynucleotide modification can include the introduction, deletion, or substitution of 1-75 nucleotides at each target sequence of said polynucleotide of said cell(s). The modification can include the introduction, deletion, or substitution of at least 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of at least 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of at least 20, 21, 22, 23, 24, 25, 26, 27, 28, 29, 30, 35, 40, 45, 50, or 75 nucleotides at each target sequence of said cell(s). The modification can include the introduction, deletion, or substitution of at least 40, 45, 50, 75, 100, 200, 300, 400 or 500 nucleotides at each target sequence of said cell(s). The modification can include the introduction, deletion, or substitution of at least 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, or 9900 to 10000 nucleotides at each target sequence of said cell(s).

In one embodiment, the modifications can include the introduction, deletion, or substitution of nucleotides at each target sequence of said cell(s) via nucleic acid components (e.g., guide(s) RNA(s)), such as those mediated by a composition, system, or a component thereof described elsewhere herein. In one embodiment, the modifications can include the introduction, deletion, or substitution of nucleotides at a target or random sequence of said cell(s) via a composition, system, or technique.

In one embodiment, the composition, system, or component thereof can promote Non-Homologous End-Joining (NHEJ). In one embodiment, modification of a polynucleotide by a composition, system, or a component thereof, such as a diseased polynucleotide, can include NHEJ. In one embodiment, promotion of this repair pathway by the composition, system, or a component thereof can be used to target gene or polynucleotide specific knock-outs and/or knock-ins. In one embodiment, promotion of this repair pathway by the composition, system, or a component thereof can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In one embodiment thee indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, or 500 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In one embodiment, composition, system, mediated NHEJ can be used in the method to delete small sequence motifs. In one embodiment, composition, system, mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a guide RNA and Cas5-HNH or Cas8-HNH polypeptide nuclease generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two guide RNAs complexing with one or more nickases induce two single strand breaks for the purpose of inducing NHEJ-mediated indels, two guide RNAs may be configured to position two single-strand breaks to provide for NHEJ repair a nucleotide of the target position.

For minimization of toxicity and off-target effect, it may be important to control the concentration of Cas5-HNH or Cas8-HNH polypeptide nuclease mRNA and guide RNA delivered. Optimal concentrations of Cas5-HNH or Cas8-HNH polypeptide nuclease mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, nickase mRNA (for example S. pyogenes Cas9 with the D10A mutation) can be delivered with a pair of guide RNAs targeting a site of interest. Guide sequences and strategies to minimize toxicity and off-target effects can be as in International Patent Publication No. WO 2014/093622 (PCT/US2013/074667); or, via mutation. Others are as described elsewhere herein.

Typically, in the context of an endogenous Cas5-HNH or Cas8-HNH polypeptide nuclease, formation of a Cas5-HNH or Cas8-HNH polypeptide nuclease or complex (comprising a guide sequence hybridized to a target sequence and complexed with one or more Cas5-HNH or Cas8-HNH polypeptide nucleases) results in cleavage, nicking, and/or another modification of one or both strands in or near (e.g., within 1, 2, 3, 4, 5, 6, 7, 8, 9, 10, 20, 50, or more base pairs from) the target sequence.

In one embodiment, a method of modifying a target polynucleotide in a cell to treat or prevent a disease can include allowing a composition, system, or component thereof to bind to the target polynucleotide, e.g., to effect cleavage, nicking, or other modification as the composition, system, is capable of said target polynucleotide, thereby modifying the target polynucleotide, wherein the composition, system, or component thereof, complex with a guide sequence, and hybridize said guide sequence to a target sequence within the target polynucleotide, wherein said guide sequence is optionally linked to a guide RNA scaffold sequence. In some of these embodiments, the composition, system, or component thereof can be or include a Cas5-HNH or Cas8-HNH polypeptide nuclease complexed with a guide sequence. In one embodiment, modification can include cleaving or nicking one or two strands at the location of the target sequence by one or more components of the composition, system, or component thereof.

The cleavage, nicking, or other modification capable of being performed by the composition, system, can modify transcription of a target polynucleotide. In one embodiment, modification of transcription can include decreasing transcription of a target polynucleotide. In one embodiment, modification can include increasing transcription of a target polynucleotide. In one embodiment, the method includes repairing said cleaved target polynucleotide by homologous recombination with a recombination template polynucleotide, wherein said repair results in a modification such as, but not limited to, an insertion, deletion, or substitution of one or more nucleotides of said target polynucleotide. In one embodiment, said modification results in one or more amino acid changes in a protein expressed from a gene comprising the target sequence. In one embodiment, the modification imparted by the composition, system, or component thereof provides a transcript and/or protein that can correct a disease or a symptom thereof, including but not limited to, any of those described in greater detail elsewhere herein.

In one embodiment, the method of treating or preventing a disease can include delivering one or more vectors or vector systems to a cell, such as a eukaryotic or prokaryotic cell, wherein one or more vectors or vector systems include the composition, system, or component thereof. In one embodiment, the vector(s) or vector system(s) can be a viral vector or vector system, such as an AAV or lentiviral vector system, which are described in greater detail elsewhere herein. In one embodiment, the method of treating or preventing a disease can include delivering one or more viral particles, such as an AAV or lentiviral particle, containing the composition, system, or component thereof. In one embodiment, the viral particle has a tissue specific tropism. In one embodiment, the viral particle has a liver, muscle, eye, heart, pancreas, kidney, neuron, epithelial cell, endothelial cell, astrocyte, glial cell, immune cell, or red blood cell specific tropism.

It will be understood that the composition and system, according to the invention as described herein, such as the composition and system, for use in the methods according to the invention as described herein, may be suitably used for any type of application known for composition, system, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In one embodiment, the composition, system, and/or component thereof described herein can be used to treat and/or prevent a circulatory system disease. Exemplary disease is provided, for example, in Table 6. In one embodiment the plasma exosomes of Wahlgren et al. (Nucleic Acids Research, 2012, Vol. 40, No. 17 e130) can be used to deliver the composition, system, and/or component thereof described herein to the blood. In one embodiment, the circulatory system disease can be treated by using a lentivirus to deliver the composition, system, described herein to modify hematopoietic stem cells (HSCs) in vivo or ex vivo (see e.g. Drakopoulou, “Review Article, The Ongoing Challenge of Hematopoietic Stem Cell-Based Gene Therapy for-Thalassemia,” Stem Cells International, Volume 2011, Article ID 987980, 10 pages, doi: 10.4061/2011/987980, which can be adapted for use with the composition, system, herein in view of the description herein). In one embodiment, the circulatory system disorder can be treated by correcting HSCs as to the disease using a composition, system, herein or a component thereof, wherein the composition, system, optionally includes a suitable HDR repair template (see e.g. Cavazzana, “Outcomes of Gene Therapy for β-Thalassemia Major via Transplantation of Autologous Hematopoietic Stem Cells Transduced Ex Vivo with a Lentiviral BA-T87Q-Globin Vector.”; Cavazzana-Calvo, “Transfusion independence and HMGA2 activation after gene therapy of human β-thalassaemia”, Nature 467, 318-322 (16 Sep. 2010) doi: 10.1038/nature09328; Nienhuis, “Development of Gene Therapy for Thalassemia, Cold Spring Harbor Perspectives in Medicine, doi: 10.1101/cshperspect.a011833 (2012), LentiGlobin BB305, a lentiviral vector containing an engineered β-globin gene (BA-T87Q); and Xie et al., “Seamless gene correction of β-thalassaemia mutations in patient-specific iPSCs using CRISPR/Cas9 and piggyback” Genome Research gr. 173427.114 (2014) www.genome.org/cgi/doi/10.1101/gr.173427.114 (Cold Spring Harbor Laboratory Press; Watts, “Hematopoietic Stem Cell Expansion and Gene Therapy” Cytotherapy 13 (10): 1164-1171. doi: 10.3109/14653249.2011.620748 (2011), which can be adapted for use with the composition, system, herein in view of the description herein). In one embodiment, iPSCs can be modified using a composition, system, described herein to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1;24 (9): 1053-65. doi: 10.1089/scd.2014.0347. Epub 2015 Feb. 5) with respect to modifying iPSCs can be adapted for use in view of the description herein with the composition, system, described herein.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit,—the receptor for stem cell factor. Hematopoietic stem cells are negative for the markers that are used for detection of lineage commitment, and are, thus, called Lin−; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, I17Ra, CD3, CD4, CD5, CD8 for T cells, etc. Mouse HSC markers: CD34lo/−, SCA-1+, Thy 1.1+/lo, CD38+, C-kit+, lin−, and Human HSC markers: CD34+, CD59+, Thyl/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34−/CD38−. Stem cells that may lack c-kit on the cell surface that are considered in the art as HSCs are within the ambit of the invention, as well as CD133+ cells likewise considered HSCs in the art.

In one embodiment, the treatment or prevention for treating a circulatory system or blood disease can include modifying a human cord blood cell with any modification described herein. In one embodiment, the treatment or prevention for treating a circulatory system or blood disease can include modifying a granulocyte colony-stimulating factor-mobilized peripheral blood cell (mPB) with any modification described herein. In one embodiment, the human cord blood cell or mPB can be CD34+. In one embodiment, the cord blood cell(s) or mPB cell(s) modified can be autologous. In one embodiment, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The compositions may be engineered to target genetic locus or loci in HSCs. In one embodiment, the Cas5-HNH or Cas8-HNH polypeptide nuclease(s) can be codon-optimized for a eukaryotic cell and especially a mammalian cell, e.g., a human cell, for instance, HSC, or iPSC and hRNA targeting a locus or loci in HSC, such as circulatory disease, can be prepared. These may be delivered via particles. The particles may be formed by the Cas5-HNH or Cas8-HNH polypeptide nuclease and the guide RNA being admixed. The guide RNA and Cas5-HNH or Cas8-HNH polypeptide nuclease mixture can be, for example, admixed with a mixture comprising or consisting essentially of or consisting of surfactant, phospholipid, biodegradable polymer, lipoprotein and alcohol, whereby particles containing the guide RNA and Cas5-HNH or Cas8-HNH polypeptide nuclease may be formed. The invention comprehends so making particles and particles from such a method as well as uses thereof. Particles suitable delivery of the composition in the context of blood or circulatory system or HSC delivery to the blood or circulatory system are described in greater detail elsewhere herein.

In one embodiment, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16;121 (20): 4082-9. doi: 10.1182/blood-2012-09-455204. Epub 2013 Mar. 21.

In one embodiment, the HSCs or iPSCs modified can be autologous. In one embodiment, the HSCs or iPSCs can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein.

### Treating Neurological Diseases

In one embodiment, the compositions, systems, described herein can be used to treat diseases of the brain and CNS. Delivery options for the brain include encapsulation of Cas5-HNH or Cas8-HNH polypeptide nuclease and guide RNA in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing Cas5-HNH or Cas8-HNH polypeptide nuclease and guide RNA. For instance, Xia C F and Boado R J, Pardridge W M (“Antibody-mediated targeting of siRNA via the human insulin receptor using avidin-biotin technology.” Mol Pharm. 2009 May-June;6 (3): 747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the compositions, systems, herein. In other embodiments, an artificial virus can be generated for CNS and/or brain delivery. See e.g., Zhang et al. (Mol Ther. 2003 January;7 (1): 11-8.)), the teachings of which can be adapted for use with the compositions, systems, herein.

### Treating Hearing Diseases

In one embodiment, the composition and system described herein can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In one embodiment, the composition, system, or modified cells can be delivered to one or both ears for treating or preventing hearing disease or loss by any suitable method or technique. Suitable methods and techniques include, but are not limited to, those set forth in US Patent Publication No. 20120328580, which describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the compounds described herein can be administered by intratympanic injection (e.g., into the middle ear), and/or injections into the outer, middle, and/or inner ear; administration in situ, via a catheter or pump (see e.g. McKenna et al., (U.S. Patent Publication No. 2006/0030837) and Jacobsen et al., (U.S. Pat. No. 7,206,639); administration in combination with a mechanical device such as a cochlear implant or a hearing aid, which is worn in the outer ear (see e.g., U.S. Patent Publication No. 2007/0093878, which provides an exemplary cochlear implant suitable for delivery of the compositions, systems, described herein to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In one embodiment, a catheter or pump can be positioned, e.g., in the ear (e.g., the outer, middle, and/or inner ear) of a patient during a surgical procedure. In one embodiment, a catheter or pump can be positioned, e.g., in the ear (e.g., the outer, middle, and/or inner ear) of a patient without the need for a surgical procedure.

In general, the cell therapy methods described in US Patent Publication No. 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Patent Publication No. 2005/0287127) and Li et al., (U.S. patent application Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318 (5858): 1917-1920 (2007); Nakagawa et al., Nat. Biotechnol. 26:101-106 (2008); and Zachres and Scholer, Cell 131 (5): 834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The composition and system may be delivered to the ear by direct application of pharmaceutical composition to the outer ear, with compositions modified from US Patent Publication No. 20110142917. In one embodiment, the pharmaceutical composition is applied to the ear canal. Delivery to the ear may also be referred to as aural or otic delivery.

In one embodiment, the compositions, systems, or components thereof and/or vectors or vector systems can be delivered to ear via a transfection to the inner ear through the intact round window by a novel proteidic delivery technology which may be applied to the nucleic acid-targeting system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June;228 (1-2): 180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present invention for delivery to the ear.

In one embodiment, the system set forth in Mukherjea et al. (Antioxidants & Redox Signaling, Volume 13, Number 5, 2010) can be adapted for transtympanic administration of the composition, system, or component thereof to the ear. In one embodiment, a dosage of about 2 mg to about 4 mg of Cas5-HNH or Cas8-HNH polypeptide nuclease for administration to a human.

In one embodiment, the system set forth in Jung et al. (Molecular Therapy, vol. 21 no. 4, 834-841 April 2013) can be adapted for vestibular epithelial delivery of the composition, system, or component thereof to the ear. In one embodiment, a dosage of about 1 to about 30 mg of Cas5-HNH or Cas8-HNH polypeptide nuclease for administration to a human.

### Treating Diseases in Non-Dividing Cells

In one embodiment, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U2OS) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a Cas5-HNH or Cas8-HNH polypeptide nuclease-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the compositions, systems, described herein.

Thus, reactivation of HR in cells, especially non-dividing, fully differentiated cell types is preferred. In one embodiment, promotion of the BRCA1-PALB2 interaction is preferred. In one embodiment, the target ell is a non-dividing cell. In one embodiment, the target cell is a neuron or muscle cell. In one embodiment, the target cell is targeted in vivo. In one embodiment, the cell is in G1 and HR is suppressed. In one embodiment, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred. In one embodiment, especially where the target cells are non-dividing, or where removal and return (ex vivo gene targeting) is problematic, for example neuron or muscle cells. KEAP1 siRNA is available from ThermoFischer. In one embodiment, a BRCA1-PALB2 complex may be delivered to the G1 cell. In one embodiment, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In one embodiment, the disease to be treated is a disease that affects the eyes. Thus, in one embodiment, the composition, system, or component thereof described herein is delivered to one or both eyes.

The composition, system, can be used to correct ocular defects that arise from several genetic mutations further described in Genetic Diseases of the Eye, Second Edition, edited by Elias I. Traboulsi, Oxford University Press, 2012.

In one embodiment, the condition to be treated or targeted is an eye disorder. In one embodiment, the eye disorder may include glaucoma. In one embodiment, the eye disorder includes a retinal degenerative disease. In one embodiment, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In one embodiment, the retinal degenerative disease is Leber Congenital Amaurosis (LCA) or Retinitis Pigmentosa. Other exemplary eye diseases are described in greater detail elsewhere herein.

In one embodiment, the composition, system, is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In one embodiment, for administration to the eye, lentiviral vectors. In one embodiment, the lentiviral vector is an equine infectious anemia virus (EIAV) vector. Exemplary EIAV vectors for eye delivery are described in Balagaan, J Gene Med 2006; 8:275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the composition, system, described herein. In one embodiment, the dosage can be 1.1×10⁵ transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 April 2011; Dalkara et al. (Sci Transl Med 5, 189ra76 (2013)), which can be adapted for use with the composition, system, described herein. In one embodiment, the dose can range from about 10⁶ to 10^{9.5} particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human.

In one embodiment, the sd-rxRNA® system of R×i Pharmaceuticals may be used/and or adapted for delivering composition, system, to the eye. In this system, a single intravitreal administration of 3 μg of sd-rxRNA results in sequence-specific reduction of PPIB mRNA levels for 14 days. The sd-rxRNA® system may be applied to the nucleic acid-targeting system of the present invention, contemplating a dose of about 3 to 20 mg of composition administered to a human.

In other embodiments, the methods of US Patent Publication No. 20130183282, which is directed to methods of cleaving a target sequence from the human rhodopsin gene, may also be modified to the nucleic acid-targeting system of the present invention.

In other embodiments, the methods of US Patent Publication No. 20130202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye may be used or adapted. In particular, desirable targets are zgc: 193933, prdmla, spata2, tex10, rbb4, ddx3, zp2.2, Blimp-1 and HtrA2, all of which may be targeted by the composition, system, of the present invention.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the compositions, systems, described herein.

US Patent Publication No. 20120159653 describes use of zinc finger nucleases to genetically modify cells, animals and proteins associated with macular degeneration (MD), the teachings of which can be applied to and/or adapted for the compositions, systems, described herein.

One aspect of US Patent Publication No. 20120159653 relates to editing of any chromosomal sequences that encode proteins associated with MD which may be applied to the nucleic acid-targeting system of the present invention.

### Treating Muscle Diseases and Cardiovascular Diseases

In one embodiment, the composition, system can be used to treat and/or prevent a muscle disease and associated circulatory or cardiovascular disease or disorder. The present invention also contemplates delivering the composition, system, described herein, e.g., Cas5-HNH or Cas8-HINH polypeptide systems, to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×10¹⁴ vector genomes is contemplated for systemic administration. See also, e.g., Eulalio et al. (2012) Nature 492:376 and Somasuntharam et al. (2013) Biomaterials 34:7790, the teachings of which can be adapted for and/or applied to the compositions, systems, described herein.

For example, US Patent Publication No. 20110023139, the teachings of which can be adapted for and/or applied to the compositions, systems, described herein, describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR).

The compositions, systems, herein can be used for treating diseases of the muscular system. The present invention also contemplates delivering the composition, system, described herein, effector protein systems, to muscle(s).

In one embodiment, the muscle disease to be treated is a muscle dystrophy such as DMD. In one embodiment, the composition, system, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using a composition, system, described herein capable of RNA modification. In one embodiment, exon skipping can be achieved in dystrophin mRNA. In one embodiment, the composition, system, can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In one embodiment, the composition, system, can induce exon skipping at exon 43, 44, 50, 51, 52, 55, or any combination thereof of the dystrophin mRNA. Mutations in these exons, can also be corrected using non-exon skipping polynucleotide modification methods.

In one embodiment, for treatment of a muscle disease, the method of Bortolanza et al. Molecular Therapy vol. 19 no. 11, 2055-264 November 2011) may be applied to an AAV expressing Cas5-HNH or Cas8-HNH polypeptide nuclease and injected into humans at a dosage of about 2×10¹⁵ or 2×10¹⁶ vg of vector. The teachings of Bortolanza et al., can be adapted for and/or applied to the compositions, systems, described herein.

In one embodiment, the method of Dumonceaux et al. (Molecular Therapy vol. 18 no. 5, 881-887 May 2010) may be applied to an AAV expressing Cas5-HNH or Cas8-HNH polypeptide nuclease and injected into humans, for example, at a dosage of about 10¹⁴ to about 10¹⁵ vg of vector. The teachings of Dumonceaux described herein can be adapted for and/or applied to the compositions, systems, described herein.

In one embodiment, the method of Kinouchi et al. (Gene Therapy (2008) 15, 1126-1130) may be applied to compositions described herein and injected into a human, for example, at a dosage of about 500 to 1000 ml of a 40 μM solution into the muscle.

In one embodiment, the method of Hagstrom et al. (Molecular Therapy Vol. 10, No. 2, August 2004) can be adapted for and/or applied to the compositions, systems, herein and injected at a dose of about 15 to about 50 mg into the great saphenous vein of a human.

In one embodiment, the method comprises treating a sickle cell related disease, e.g., sickle cell trait, sickle cell disease such as sickle cell anemia, β-thalassaemia. For example, the method and system may be used to modify the genome of the sickle cell, e.g., by correcting one or more mutations of the β-globin gene. In the case of β-thalassaemia, sickle cell anemia can be corrected by modifying HSCs with the systems. The system allows the specific editing of the cell's genome by cutting its DNA and then letting it repair itself. The Cas5-HNH or Cas8-HNH polypeptide nuclease is inserted and directed by an RNA guide to the mutated point and then it cuts the DNA at that point. Simultaneously, a healthy version of the sequence is inserted. This sequence is used by the cell's own repair system to fix the induced cut. In this way, the Cas5-HNH or Cas8-HNH polypeptide nuclease allows the correction of the mutation in the previously obtained stem cells. The methods and systems may be used to correct HSCs as to sickle cell anemia using a systems that targets and corrects the mutation (e.g., with a suitable HDR template that delivers a coding sequence for β-globin, advantageously non-sickling β-globin); specifically, the guide RNA can target mutation that give rise to sickle cell anemia, and the HDR can provide coding for proper expression of β-globin. A guide RNA that targets the mutation-and-Cas5-HNH or Cas8-HNH polypeptide nuclease containing particle is contacted with HSCs carrying the mutation. The particle also can contain a suitable HDR template to correct the mutation for proper expression of β-globin; or the HSC can be contacted with a second particle or a vector that contains or delivers the HDR template. The so contacted cells can be administered; and optionally treated/expanded; cf. Cartier. The HDR template can provide for the HSC to express an engineered β-globin gene (e.g., βA-T87Q), or β-globin.

### Treating Diseases of the Liver and Kidney

In one embodiment, the composition, system, or component thereof described herein can be used to treat a disease of the kidney or liver. Thus, in one embodiment, delivery of the composition or component thereof described herein is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Révész and Péter Hamar (2011). Delivery Methods to Target RNAs in the Kidney, Gene Therapy Applications, Prof. Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the composition of the present invention contemplating a 1-2 g subcutaneous injection of Cas5-HNH or Cas8-HNH polypeptide nuclease conjugated with cholesterol to a human for delivery to the kidneys. In one embodiment, the method of Molitoris et al. (J Am Soc Nephrol 20:1754-1764, 2009) can be adapted to the composition and a cumulative dose of 12-20 mg/kg to a human can be used for delivery to the proximal tubule cells of the kidneys. In one embodiment, the methods of Thompson et al. (Nucleic Acid Therapeutics, Volume 22, Number 4, 2012) can be adapted to the compositions and a dose of up to 25 mg/kg can be delivered via i.v. administration. In one embodiment, the method of Shimizu et al. (J Am Soc Nephrol 21:622-633, 2010) can be adapted to the compositions and a dose of about of 10-20 μmol compositions complexed with nanocarriers in about 1-2 liters of a physiologic fluid for i.v. administration can be used.

Other various delivery vehicles can be used to deliver the composition, system to the kidney such as viral, hydrodynamic, lipid, polymer nanoparticles, aptamers and various combinations thereof (see e.g. Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In one embodiment, delivery is to liver cells. In one embodiment, the liver cell is a hepatocyte. Delivery of the composition and system herein may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted recombination template) to be achieved even if only a small fraction of hepatocytes are edited. See sites identified by Wechsler et al. (reported at the 57th Annual Meeting and Exposition of the American Society of Hematology-abstract available online at ash.confex.com/ash/2015/webprogram/Paper86495.html and presented on 6Dec. 2015) which can be adapted for use with the compositions, systems, herein.

Exemplary liver and kidney diseases that can be treated and/or prevented are described elsewhere herein.

### Treating Epithelial and Lung Diseases

In one embodiment, the disease treated or prevented by the composition and system described herein can be a lung or epithelial disease. The compositions and systems described herein can be used for treating epithelial and/or lung diseases. The present invention also contemplates delivering the composition, system, described herein, to one or both lungs.

In one embodiment, as viral vector can be used to deliver the composition, system, or component thereof to the lungs. In one embodiment, the AAV is an AAV-1, AAV-2, AAV-5, AAV-6, and/or AAV-9 for delivery to the lungs. (see, e.g., Li et al., Molecular Therapy, vol. 17 no. 12, 2067-277 December 2009). In one embodiment, the MOI can vary from 1×10³ to 4×10⁵ vector genomes/cell. In one embodiment, the delivery vector can be an RSV vector as in Zamora et al. (Am J Respir Crit Care Med Vol 183. pp 531-538, 2011. The method of Zamora et al. may be applied to the nucleic acid-targeting system of the present invention and an aerosolized composition, for example with a dosage of 0.6 mg/kg, may be contemplated for the present invention.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1a promoter for Cas and U6 or H1 promoter for guide RNA. A preferred arrangement is to use a CFTRdelta508 targeting guide, a repair template for deltaF508 mutation and a codon optimized composition, with optionally one or more nuclear localization signal or sequence(s) (NLS(s)), e.g., two (2) NLSs.

### Treating Diseases of the Skin

The compositions and systems described herein can be used for the treatment of skin diseases. The present invention also contemplates delivering the composition and system, described herein, to the skin.

In one embodiment, delivery to the skin (intradermal delivery) of the composition, system, or component thereof can be via one or more microneedles or microneedle containing device. For example, in one embodiment the device and methods of Hickerson et al. (Molecular Therapy-Nucleic Acids (2013) 2, e129) can be used and/or adapted to deliver the composition, system, described herein, for example, at a dosage of up to 300 μl of 0.1 mg/ml compositions to the skin.

In one embodiment, the methods and techniques of Leachman et al. (Molecular Therapy, vol. 18 no. 2, 442-446 February 2010) can be used and/or adapted for delivery of a compositions described herein to the skin.

In one embodiment, the methods and techniques of Zheng et al. (PNAS, Jul. 24, 2012, vol. 109, no. 30, 11975-11980) can be used and/or adapted for nanoparticle delivery of a compositions described herein to the skin. In one embodiment, as dosage of about 25 nM applied in a single application can achieve gene knockdown in the skin.

### Treating Cancer

The compositions, systems, described herein can be used for the treatment of cancer. The present invention also contemplates delivering the composition, system, described herein, to a cancer cell. Also, as is described elsewhere herein the compositions, systems, can be used to modify an immune cell, such as a CAR or CAR T cell, which can then in turn be used to treat and/or prevent cancer. This is also described in International Patent Publication No. WO 2015/161276, the disclosure of which is hereby incorporated by reference and described herein below.

Target genes suitable for the treatment or prophylaxis of cancer can include those set forth in Tables 7 and 8. In one embodiment, target genes for cancer treatment and prevention can also include those described in International Patent Publication No. WO 2015/048577 the disclosure of which is hereby incorporated by reference and can be adapted for and/or applied to the composition, system, described herein.

### Adoptive Cell Therapy

The compositions, systems, and components thereof described herein can be used to modify cells for an adoptive cell therapy. In an aspect of the invention, methods and compositions which involve editing a target nucleic acid sequence, or modulating expression of a target nucleic acid sequence, and applications thereof in connection with cancer immunotherapy are comprehended by adapting the composition, system, of the present invention. In some examples, the compositions, systems, and methods may be used to modify a stem cell (e.g., induced pluripotent cell) to derive modified natural killer cells, gamma delta T cells, and alpha beta T cells, which can be used for the adoptive cell therapy. In certain examples, the compositions, systems, and methods may be used to modify modified natural killer cells, gamma delta T cells, and alpha beta T cells.

As used herein, “ACT”, “adoptive cell therapy” and “adoptive cell transfer” may be used interchangeably. In an embodiment, Adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4;8 (1): 424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing GVHD issues. The adoptive transfer of autologous tumor infiltrating lymphocytes (TIL) (Zacharakis et al., (2018) Nat Med. 2018 June;24 (6): 724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57.) or genetically re-directed peripheral blood mononuclear cells (Johnson et al., (2009) Blood 114 (3): 535-46; and Morgan et al., (2006) Science 314 (5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In an embodiment, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32:189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12 (4): 269-281; and Jenson and Riddell, 2014, Design and implementation of adoptive therapy with chimeric antigen receptor-modified T cells. Immunol Rev. 257 (1): 127-144; and Rajasagi et al., 2014, Systematic identification of personal tumor-specific neoantigens in chronic lymphocytic leukemia. Blood. 2014 Jul. 17;124 (3): 453-62).

In an embodiment, an antigen (such as a tumor antigen) to be targeted in adoptive cell therapy (such as particularly CAR or TCR T-cell therapy) of a disease (such as particularly of tumor or cancer) may be selected from a group consisting of: MR1 (see, e.g., Crowther, et al., 2020, Genome-wide CRISPR-Cas9 screening reveals ubiquitous T cell cancer targeting via the monomorphic MHC class I-related protein MR1, Nature Immunology vol. 21, pages178-185), B cell maturation antigen (BCMA) (see, e.g., Friedman et al., Effective Targeting of Multiple BCMA-Expressing Hematological Malignancies by Anti-BCMA CAR T Cells, Hum Gene Ther. 2018 Mar. 8; Berdeja J G, et al. Durable clinical responses in heavily pretreated patients with relapsed/refractory multiple myeloma: updated results from a multicenter study of bb2121 anti-Bcma CAR T cell therapy. Blood. 2017; 130:740; and Mouhieddine and Ghobrial, Immunotherapy in Multiple Myeloma: The Era of CAR T Cell Therapy, Hematologist, May-June 2018, Volume 15, issue 3); PSA (prostate-specific antigen); prostate-specific membrane antigen (PSMA); PSCA (Prostate stem cell antigen); Tyrosine-protein kinase transmembrane receptor ROR1; fibroblast activation protein (FAP); Tumor-associated glycoprotein 72 (TAG72); Carcinoembryonic antigen (CEA); Epithelial cell adhesion molecule (EPCAM); Mesothelin; Human Epidermal growth factor Receptor 2 (ERBB2 (Her2/neu)); Prostase; Prostatic acid phosphatase (PAP); elongation factor 2 mutant (ELF2M); Insulin-like growth factor 1 receptor (IGF-1R); gp100; BCR-ABL (breakpoint cluster region-Abelson); tyrosinase; New York esophageal squamous cell carcinoma 1 (NY-ESO-1); k-light chain, LAGE (L antigen); MAGE (melanoma antigen); Melanoma-associated antigen 1 (MAGE-A1); MAGE A3; MAGE A6; legumain; Human papillomavirus (HPV) E6; HPV E7; prostein; survivin; PCTA1 (Galectin 8); Melan-A/MART-1; Ras mutant; TRP-1 (tyrosinase related protein 1, or gp75); Tyrosinase-related Protein 2 (TRP2); TRP-2/INT2 (TRP-2/intron 2); RAGE (renal antigen); receptor for advanced glycation end products 1 (RAGE1); Renal ubiquitous 1, 2 (RU1, RU2); intestinal carboxyl esterase (iCE); Heat shock protein 70-2 (HSP70-2) mutant; thyroid stimulating hormone receptor (TSHR); CD123; CD171; CD19; CD20; CD22; CD26; CD30; CD33; CD44v7/8 (cluster of differentiation 44, exons 7/8); CD53; CD92; CD100; CD148; CD150; CD200; CD261; CD262; CD362; CS-1 (CD2 subset 1, CRACC, SLAMF7, CD319, and 19A24); C-type lectin-like molecule-1 (CLL-1); ganglioside GD3 (aNeu5Ac (2-8) aNeu5Ac (2-3) bDGalp (1-4) bDGlcp (1-1) Cer); Tn antigen (Tn Ag); Fms-Like Tyrosine Kinase 3 (FLT3); CD38; CD138; CD44v6; B7H3 (CD276); KIT (CD117); Interleukin-13 receptor subunit alpha-2 (IL-13Ra2); Interleukin 11 receptor alpha (IL-11Ra); prostate stem cell antigen (PSCA); Protease Serine 21 (PRSS21); vascular endothelial growth factor receptor 2 (VEGFR2); Lewis (Y) antigen; CD24; Platelet-derived growth factor receptor beta (PDGFR-beta); stage-specific embryonic antigen-4 (SSEA-4); Mucin 1, cell surface associated (MUC1); mucin 16 (MUC16); epidermal growth factor receptor (EGFR); epidermal growth factor receptor variant III (EGFRvIII); neural cell adhesion molecule (NCAM); carbonic anhydrase IX (CAIX); Proteasome (Prosome, Macropain) Subunit, Beta Type, 9 (LMP2); ephrin type-A receptor 2 (EphA2); Ephrin B2; Fucosyl GM1; sialyl Lewis adhesion molecule (sLe); ganglioside GM3 (aNeu5Ac (2-3) bDGalp (1-4) bDGlcp (1-1) Cer); TGS5; high molecular weight-melanoma-associated antigen (HMWMAA); 0-acetyl-GD2 ganglioside (OAcGD2); Folate receptor alpha; Folate receptor beta; tumor endothelial marker 1 (TEM1/CD248); tumor endothelial marker 7-related (TEM7R); claudin 6 (CLDN6); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoHI glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); CT (cancer/testis (antigen)); melanoma cancer testis antigen-1 (MAD-CT-1); melanoma cancer testis antigen-2 (MAD-CT-2); Fos-related antigen 1; p53; p53 mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; Cyclin D1; v-myc avian myelocytomatosis viral oncogene neuroblastoma derived homolog (MYCN); Ras Homolog Family Member C (RhoC); Cytochrome P450 1B1 (CYP1B1); CCCTC-Binding Factor (Zinc Finger Protein)-Like (BORIS); Squamous Cell Carcinoma Antigen Recognized By T Cells-1 or 3 (SART1, SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint-1,-2,-3 or 4 (SSX1, SSX2, SSX3, SSX4); CD79a; CD79b; CD72; Leukocyte-associated immunoglobulin-like receptor 1 (LAIR1); Fc fragment of IgA receptor (FCAR); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); mouse double minute 2 homolog (MDM2); livin; alphafetoprotein (AFP); transmembrane activator and CAML Interactor (TACI); B-cell activating factor receptor (BAFF-R); V-Ki-ras2 Kirsten rat sarcoma viral oncogene homolog (KRAS); immunoglobulin lambda-like polypeptide 1 (IGLL1); 707-AP (707 alanine proline); ART-4 (adenocarcinoma antigen recognized by T4 cells); BAGE (B antigen; b-catenin/m, b-catenin/mutated); CAMEL (CTL-recognized antigen on melanoma); CAP1 (carcinoembryonic antigen peptide 1); CASP-8 (caspase-8); CDC27m (cell-division cycle 27 mutated); CDK4/m (cycline-dependent kinase 4 mutated); Cyp-B (cyclophilin B); DAM (differentiation antigen melanoma); EGP-2 (epithelial glycoprotein 2); EGP-40 (epithelial glycoprotein 40); Erbb2, 3, 4 (erythroblastic leukemia viral oncogene homolog-2,-3, 4); FBP (folate binding protein); fAchR (Fetal acetylcholine receptor); G250 (glycoprotein 250); GAGE (G antigen); GnT-V (N-acetylglucosaminyltransferase V); HAGE (helicose antigen); ULA-A (human leukocyte antigen-A); HST2 (human signet ring tumor 2); KIAA0205; KDR (kinase insert domain receptor); LDLR/FUT (low density lipid receptor/GDP L-fucose: b-D-galactosidase 2-a-L fucosyltransferase); LICAM (L1 cell adhesion molecule); MC1R (melanocortin 1 receptor); Myosin/m (myosin mutated); MUM-1,-2,-3 (melanoma ubiquitous mutated 1, 2, 3); NA88-A (NA cDNA clone of patient M88); KG2D (Natural killer group 2, member D) ligands; oncofetal antigen (h5T4); p190 minor bcr-abl (protein of 190KD bcr-abl); Pml/RARa (promyelocytic leukemia/retinoic acid receptor a); PRAME (preferentially expressed antigen of melanoma); SAGE (sarcoma antigen); TEL/AML1 (translocation Ets-family leukemia/acute myeloid leukemia 1); TPI/m (triosephosphate isomerase mutated); CD70; and any combination thereof.

In an embodiment, an antigen to be targeted in adoptive cell therapy (such as particularly CAR or TCR T-cell therapy) of a disease (such as particularly of tumor or cancer) is a tumor-specific antigen (TSA).

In an embodiment, an antigen to be targeted in adoptive cell therapy (such as particularly CAR or TCR T-cell therapy) of a disease (such as particularly of tumor or cancer) is a neoantigen.

In an embodiment, an antigen to be targeted in adoptive cell therapy (such as particularly CAR or TCR T-cell therapy) of a disease (such as particularly of tumor or cancer) is a tumor-associated antigen (TAA).

In an embodiment, an antigen to be targeted in adoptive cell therapy (such as particularly CAR or TCR T-cell therapy) of a disease (such as particularly of tumor or cancer) is a universal tumor antigen. In certain preferred embodiments, the universal tumor antigen is selected from the group consisting of: a human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (DI), and any combinations thereof.

In an embodiment, an antigen (such as a tumor antigen) to be targeted in adoptive cell therapy (such as particularly CAR or TCR T-cell therapy) of a disease (such as particularly of tumor or cancer) may be selected from a group consisting of: CD19, BCMA, CD70, CLL-1, MAGE A3, MAGE A6, HPV E6, HPV E7, WT1, CD22, CD171, ROR1, MUC16, and SSX2. In certain preferred embodiments, the antigen may be CD19. For example, CD19 may be targeted in hematologic malignancies, such as in lymphomas, more particularly in B-cell lymphomas, such as without limitation in diffuse large B-cell lymphoma, primary mediastinal b-cell lymphoma, transformed follicular lymphoma, marginal zone lymphoma, mantle cell lymphoma, acute lymphoblastic leukemia including adult and pediatric ALL, non-Hodgkin lymphoma, indolent non-Hodgkin lymphoma, or chronic lymphocytic leukemia. For example, BCMA may be targeted in multiple myeloma or plasma cell leukemia (see, e.g., 2018 American Association for Cancer Research (AACR) Annual meeting Poster: Allogeneic Chimeric Antigen Receptor T Cells Targeting B Cell Maturation Antigen). For example, CLL1 may be targeted in acute myeloid leukemia. For example, MAGE A3, MAGE A6, SSX2, and/or KRAS may be targeted in solid tumors. For example, HPV E6 and/or HPV E7 may be targeted in cervical cancer or head and neck cancer. For example, WT1 may be targeted in acute myeloid leukemia (AML), myelodysplastic syndromes (MDS), chronic myeloid leukemia (CML), non-small cell lung cancer, breast, pancreatic, ovarian or colorectal cancers, or mesothelioma. For example, CD22 may be targeted in B cell malignancies, including non-Hodgkin lymphoma, diffuse large B-cell lymphoma, or acute lymphoblastic leukemia. For example, CD171 may be targeted in neuroblastoma, glioblastoma, or lung, pancreatic, or ovarian cancers. For example, ROR1 may be targeted in ROR1+ malignancies, including non-small cell lung cancer, triple negative breast cancer, pancreatic cancer, prostate cancer, ALL, chronic lymphocytic leukemia, or mantle cell lymphoma. For example, MUC16 may be targeted in MUC16ecto+ epithelial ovarian, fallopian tube or primary peritoneal cancer. For example, CD70 may be targeted in both hematologic malignancies as well as in solid cancers such as renal cell carcinoma (RCC), gliomas (e.g., GBM), and head and neck cancers (HNSCC). CD70 is expressed in both hematologic malignancies as well as in solid cancers, while its expression in normal tissues is restricted to a subset of lymphoid cell types (see, e.g., 2018 American Association for Cancer Research (AACR) Annual meeting Poster: Allogeneic CRISPR Engineered Anti-CD70 CAR-T Cells Demonstrate Potent Preclinical Activity Against Both Solid and Hematological Cancer Cells).

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO 9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in one embodiment, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3% or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD32 or scFv-CD28-OX40-CD3ζ; see U.S. Pat. Nos. 8,906,682; 8,399,645; 5,686,281; PCT Publication No. WO 2014/134165; PCT Publication No. WO 2012/079000). In an embodiment, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon R1b), CD79a, CD79b, Fc gamma RIIa, DAP10, and DAP12. In certain preferred embodiments, the primary signaling domain comprises a functional signaling domain of CD3ζ or FcRγ. In an embodiment, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAMI (CD226), SLAMF4 (CD244, 2B4), CD84, CD96 (Tactile), CEACAMI, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In an embodiment, the one or more costimulatory signaling domains comprise a functional signaling domain of a protein selected, each independently, from the group consisting of: 4-1BB, CD27, and CD28. In an embodiment, a chimeric antigen receptor may have the design as described in U.S. Pat. No. 7,446,190, comprising an intracellular domain of CD35 chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139. Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, an embodiment employs a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native aßTCR, for example by antigen on professional antigen-presenting cells, with attendant costimulation. In addition, additional engineered receptors may be provided on the immunoresponsive cells, for example to improve targeting of a T-cell attack and/or minimize side effects.

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34:1157-1165), a portion of the human CD28 molecule, and the intracellular component of the human TCR-2 molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-molecule. The exact sequence of the CD28 molecule included in the FMC63-28Z CAR corresponded to Genbank identifier NM_006139; the sequence included all amino acids starting with the amino acid sequence IEVMYPPPY (SEQ ID NO: 52) and continuing all the way to the carboxy-terminus of the protein. To encode the anti-CD19 scFv component of the vector, the authors designed a DNA sequence which was based on a portion of a previously published CAR (Cooper et al., (2003) Blood 101:1637-1644). This sequence encoded the following components in frame from the 5′ end to the 3′ end: an Xhol site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with Xhol and NotI. To form the MSGV-FMC63-28Z retroviral vector, the Xhol and NotI-digested fragment encoding the FMC63 scFv was ligated into a second Xhol and NotI-digested fragment that encoded the MSGV retroviral backbone (as in Hughes et al., (2005) Human Gene Therapy 16:457-472) as well as part of the extracellular portion of human CD28, the entire transmembrane and cytoplasmic portion of human CD28, and the cytoplasmic portion of the human TCR-ζmolecule (as in Maher et al., 2002) Nature Biotechnology 20:70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in an embodiment, cells intended for adoptive cell therapies, more particularly immunoresponsive cells such as T cells, may express the FMC63-28Z CAR as described by Kochenderfer et al. (supra). Hence, in an embodiment, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM 006139 (sequence version 1, 2 or 3) starting with the amino acid sequence IEVMYPPPY (SEQ ID NO: 52) and continuing all the way to the carboxy-terminus of the protein. Preferably, the antigen is CD19, more preferably the antigen-binding element is an anti-CD19 scFv, even more preferably the anti-CD19 scFv as described by Kochenderfer et al. (supra).

Additional anti-CD19 CARs are further described in International Patent Publication No. WO 2015/187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signaling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcγRI gamma chain; or CD28-FcγRI gamma chain) were disclosed. Hence, in an embodiment, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of No. WO 2015/187528. Preferably, the antigen is CD19, more preferably the antigen-binding element is an anti-CD19 scFv, even more preferably the mouse or human anti-CD19 scFv as described in Example 1 of. WO 2015/187528. In an embodiment, the CAR comprises, consists essentially of or consists of an amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, or SEQ ID NO: 13 as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March;78:145-150; and Jin et al., CD70, a novel target of CAR T-cell therapy for gliomas, Neuro Oncol. 2018 Jan. 10;20 (1): 55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am.J.Pathol. 1995; 147:1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In an embodiment, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In an embodiment, the chimeric inhibitory receptor comprises an extracellular antigen-binding element (or portion or domain) configured to specifically bind to a target antigen, a transmembrane domain, and an intracellular immunosuppressive or repressive signaling domain. In an embodiment, the second target antigen is an antigen that is not expressed on the surface of a cancer cell or infected cell or the expression of which is downregulated on a cancer cell or an infected cell. In an embodiment, the second target antigen is an MHC-class I molecule. In an embodiment, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in one embodiment, TCR expression may eliminated using RNA interference (e.g., shRNA, siRNA, miRNA, etc.), Cas5-HNH or Cas8-HNH polypeptide nuclease, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US Patent Publication Nos. US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLOS One, 2013) or to downregulate expression of the CAR following binding to the target antigen (International Patent Publication No. WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD32 and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In an embodiment, ACT includes co-transferring CD4+ Th1 cells and CD8+ CTLs to induce a synergistic antitumor response (see, e.g., Li et al., Adoptive cell therapy with CD4+ T helper 1 cells and CD8+ cytotoxic T cells enhances complete rejection of an established tumor, leading to generation of endogenous memory responses to non-targeted tumor epitopes. Clin Transl Immunology. 2017 October; 6 (10): e160).

In an embodiment, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112 (2): 362-73; and Martin-Orozco N, et al., T helper 17 cells promote cytotoxic T cell activation in tumor immunity. Immunity. 2009 Nov. 20; 31 (5): 787-98). Those studies involved an adoptive T cell transfer (ACT) therapy approach, which takes advantage of CD4+ T cells that express a TCR recognizing tyrosinase tumor antigen. Exploitation of the TCR leads to rapid expansion of Th17 populations to large numbers ex vivo for reinfusion into the autologous tumor-bearing hosts.

In an embodiment, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In an embodiment, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257 (1): 56-71. doi: 10.1111/imr.12132).

Approaches such as the foregoing may be adapted to provide methods of treating and/or increasing survival of a subject having a disease, such as a neoplasia, for example by administering an effective amount of an immune-responsive cell comprising an antigen recognizing receptor that binds a selected antigen, wherein the binding activates the immuno-responsive cell, thereby treating or preventing the disease (such as a neoplasia, a pathogen infection, an autoimmune disorder, or an allogeneic transplant reaction).

In an embodiment, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3 (10): 1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells, or population of cells, may be made resistant to at least one immunosuppressive agent due to the inactivation of a gene encoding a receptor for such immunosuppressive agent. In an embodiment, the immunosuppressive treatment provides for the selection and expansion of the immuno-responsive T cells within the patient.

In an embodiment, the treatment can be administered before primary treatment (e.g., surgery or radiation therapy) to shrink a tumor before the primary treatment. In another embodiment, the treatment can be administered after primary treatment to remove any remaining cancer cells.

In an embodiment, immuno-metabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immuno-responsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In one embodiment, the disclosed CARs may be delivered or administered into a cavity formed by the resection of tumor tissue (i.e., intracavity delivery) or directly into a tumor prior to resection (i.e., intratumoral delivery). In one embodiment, the cell compositions of the present invention are preferably administered by intravenous injection.

The administration of the cells or population of cells can consist of the administration of 104-109 cells per kg body weight, preferably 105 to 106 cells/kg body weight including all integer values of cell numbers within those ranges. Dosing in CAR T cell therapies may for example involve administration of from 106 to 109 cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immuno-responsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6:95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; International Patent Publication WO 2011/146862; International Patent Publication WO 2014/011987; International Patent Publication WO 2013/040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28 (6): 1107-15 (2010)).

In a further refinement of adoptive therapies, genome editing may be used to tailor immuno-responsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1;23 (9): 2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25;9 (374); Legut, et al., 2018, CRISPR-mediated TCR replacement generates superior anticancer transgenic T cells. Blood, 131 (3), 311-322; and Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018). Cells may be edited using any CRISPR system and method of use thereof as described herein. The composition and systems may be delivered to an immune cell by any method described herein. In preferred embodiments, cells are edited ex vivo and transferred to a subject in need thereof. Immuno-responsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see International Patent Publication Nos. WO 2013/176915, WO 2014/059173, WO 2014/172606, WO 2014/184744, and WO 2014/191128).

In an embodiment, editing may result in inactivation of a gene. By inactivating a gene, it is intended that the gene of interest is not expressed in a functional protein form. In a particular embodiment, the system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In an embodiment, homology directed repair (HDR) is used to concurrently inactivate a gene (e.g., TRAC) and insert an endogenous TCR or CAR into the inactivated locus.

Hence, in an embodiment, editing of cells, particularly cells intended for adoptive cell therapies, more particularly immuno-responsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543:113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in an embodiment, editing of cells, particularly cells intended for adoptive cell therapies, more particularly immuno-responsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as Cas5-HNH or Cas8-HNH system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1;112 (12): 4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In an embodiment, editing of cells, particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In an embodiment, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15;44 (2): 356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

International Patent Publication No. WO 2014/172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In an embodiment, metallothioneins are targeted by gene editing in adoptively transferred T cells.

In an embodiment, targets of gene editing may be at least one targeted locus involved in the expression of an immune checkpoint protein. Such targets may include, but are not limited to, CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL1ORA, IL1ORB, HMOX2, IL6R, IL6ST, EIF2AK4, CSK, PAGI, SIT1, FOXP3, PRDMI, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, International Patent Publication No. WO 2016/196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as the composition or system herein) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In an embodiment, cells may be engineered to express a CAR, wherein expression and/or function of methylcytosine dioxygenase genes (TET1, TET2 and/or TET3) in the cells has been reduced or eliminated, (such as the composition or system herein) (for example, as described in WO201704916).

In an embodiment, editing of cells, particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In an embodiment, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in International Patent Publication Nos. WO 2016/011210 and WO 2017/011804).

In an embodiment, editing of cells, particularly cells intended for adoptive cell therapies, more particularly immuno-responsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In an embodiment, a cell may be multiplied edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment, T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In one embodiment, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In an embodiment of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed, and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immuno-adherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (e.g., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In an embodiment, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In an embodiment, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In an embodiment, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells are minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In an embodiment, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. U.S. 2004/0224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g., via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274 (5284): 94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of 125I labeled β2-microglobulin (β2m) into MHC class I/β2m/peptide heterotrimeric complexes (see Parker et al., J. Immunol. 152:163, 1994).

In one embodiment, cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003/057171, U.S. Pat. No. 8,034,334, and U.S. Patent Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In an embodiment, T cells comprising a CAR or an exogenous TCR, may be manufactured as described in International Patent Publication No. WO 2015/120096, by a method comprising enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In an embodiment, T cells comprising a CAR or an exogenous TCR, may be manufactured as described in WO 2015/120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In an embodiment, T cell maturation or differentiation in vitro may be delayed or inhibited by the method as described in International Patent Publication No. WO 2017/070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In an embodiment, a patient in need of a T cell therapy may be conditioned by a method as described in International Patent Publication No. WO 2016/191756 comprising administering to the patient a dose of cyclophosphamide between 200 mg/m²/day and 2000 mg/m²/day and a dose of fludarabine between 20 mg/m²/day and 900 mg/m²/day.

### Diseases

Genetic Diseases and Diseases with a Genetic and/or Epigenetic Aspect

The compositions, systems, or components thereof can be used to treat and/or prevent a genetic disease or a disease with a genetic and/or epigenetic aspect. The genes and conditions exemplified herein are not exhaustive. In one embodiment, a method of treating and/or preventing a genetic disease can include administering a composition, system, and/or one or more components thereof to a subject, where the composition, system, and/or one or more components thereof is capable of modifying one or more copies of one or more genes associated with the genetic disease or a disease with a genetic and/or epigenetic aspect in one or more cells of the subject. In one embodiment, modifying one or more copies of one or more genes associated with a genetic disease or a disease with a genetic and/or epigenetic aspect in the subject can eliminate a genetic disease or a symptom thereof in the subject. In one embodiment, modifying one or more copies of one or more genes associated with a genetic disease or a disease with a genetic and/or epigenetic aspect in the subject can decrease the severity of a genetic disease or a symptom thereof in the subject. In one embodiment, the compositions, systems, or components thereof can modify one or more genes or polynucleotides associated with one or more diseases, including genetic diseases and/or those having a genetic aspect and/or epigenetic aspect, including but not limited to, any one or more set forth in the table below. It will be appreciated that those diseases and associated genes listed herein are non-exhaustive and non-limiting. Further some genes play roles in the development of multiple diseases.

TABLE 2
Exemplary Genetic and Other Diseases and Associated Genes

| | Primary Tissues | Additional | |
|---|---|---|---|
| | or System | Tissues/Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g. |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where | |
| | | homogentisic | |
| | | acid accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | | SOD1; ALS2; ALS3; ALS5; ALS7; |
| | | | STEX; FUS; TARDBP; VEGF (VEGF-a; |
| | | | VEGF-b; VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, ITPR2, |
| | | | ALS2CR4, GLS, ALS2CR8, CNTFR, |
| | | | ALS2CR11, FOLH1, FAM117B, |
| | | | P4HB, CNTF, SQSTM1, STRADB, |
| | | | NAIP, NLR, YWHAQ, SLC33A1, |
| | | | TRAK2, SCA1, NIF3L1, NIF3, |
| | | | PARD3B, COX8A, CDK15, HECW1, |
| | | | HECT, C2, WW 15, NOS1, MET, |
| | | | SOD2, HSPB1, NEFL, CTSB, ANG, |
| | | | HSPA8, RNase A, VAPB, VAMP, |
| | | | SNCA, alpha HGF, CAT, ACTB, |
| | | | NEFM, TH, BCL2, FAS, CASP3, |
| | | | CLU, SMN1, G6PD, BAX, HSF1, |
| | | | RNF19A, JUN, ALS2CR12, HSPA5, |
| | | | MAPK14, APEX1, TXNRD1, NOS2, |
| | | | TIMP1, CASP9, XIAP, GLG1, EPO, |
| | | | VEGFA, ELN, GDNF, NFE2L2, |
| | | | SLC6A3, HSPA4, APOE, PSMB8, |
| | | | DCTN2, TIMP3, KIFAP3, SLC1A1, |
| | | | SMN2, CCNC, STUB1, ALS2, |
| | | | PRDX6, SYP, CABIN1, CASP1, |
| | | | GART, CDK5, ATXN3, RTN4, |
| | | | C1QB, VEGFC, HTT, PARK7, XDH, |
| | | | GFAP, MAP2, CYCS, FCGR3B, CCS, |
| | | | UBL5, MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S:C1A3, |
| | | | FLT1, PON1, AR, LIF, ERBB3, |
| | | | :GA:S1, CD44, TP53, TLR3, GRIA1, |
| | | | GAPDH, AMPA, GRIK1, DES, |
| | | | CHAT, FLT4, CHMP2B, BAG1, |
| | | | CHRNA4, GSS, BAK1, KDR, GSTP1, |
| | | | OGG1, IL6 |
| Alzheimer's Disease | Brain | | E1; CHIP; UCH; UBB; Tau; LRP; |
| | | | PICALM; CLU; PS1; |
| | | | SORL1; CR1; VLDLR; UBA1; |
| | | | UBA3; CHIP28; AQP1; UCHL1; |
| | | | UCHL3; APP, AAA, CVAP, AD1, |
| | | | APOE, AD2, DCP1, ACE1, MPO, |
| | | | PACIP1, PAXIP1L, PTIP, A2M, |
| | | | BLMH, BMH, PSEN1, AD3, ALAS2, |
| | | | ABCA1, BIN1, BDNF, BTNL8, |
| | | | C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, IL1RN, IL- |
| | | | 1RA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| Pervasive Developmental | | | FMR1, FMR2; FXR1; FXR2; |
| Disorders (PDDs) | | | MGLUR5, ATP10C, CDH10, GRM6, |
| | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, see also |
| | | | 20110023145 |
| autosomal dominant polycystic | kidney | liver | PKD1, PKD2 |
| kidney disease (ADPKD) - | | | |
| (includes diseases such as von | | | |
| Hippel-Lindau disease and | | | |
| tuberous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Bartter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PON1, |
| | | | KNG1, CCL2, LPL, VWF, F2, |
| | | | ICAM1, TGFB, NPPA, IL10, EPO, |
| | | | SOD1, VCAM1, IFNG, LPA, MPO, |
| | | | ESR1, MAPK, HP, F3, CST3, COG2, |
| | | | MMP9, SERPINC1, F8, HMOX1, |
| | | | APOC3, IL8, PROL1, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, IL1A, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, IL1R1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RAC1, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF:AR, CA:CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADH1C, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADH1B, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDAC1, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1, HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CHRNA4, CACNA1C, PRKAG2, |
| | | | CHAT, PTGDS, NR1H2, TEK, |
| | | | VEGFB, MEF2C, MAPKAPK2, |
| | | | TNFRSF11A, HSPA9, CYSLTR1, |
| | | | MAT1A, OPRL1, IMPA1, CLCN2, |
| | | | DLD, PSMA6, PSMB8, CHI3L1, |
| | | | ALDH1B1, PARP2, STAR, LBP, |
| | | | ABCC6, RGS2, EFNB2, GJB6, |
| | | | APOA2, AMPD1, DYSF, |
| | | | FDFT1, EMD2, CCR6, GJB3, IL1RL1, |
| | | | ENTPD1, BBS4, CELSR2, F11R, |
| | | | RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2, TRH, GJC1, S:C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQP0, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT2O), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG4 (CMT4J), NT-3 |
| Chédiak-Higashi Syndrome | Immune system | Skin, hair, eyes, | LYST |
| | | neurons | |
| Choroideremia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC HNPCC: |
| adenomatous polyposis (FAP) | | | MSH2, MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POC1B, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen Storage | Various- | | AGL |
| Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g. CTNND2, TERT, |
| Cystic Fibrosis (CF) | Lungs and | Pancreas, liver, | CTFR, ABCC7, CF, MRP7, SCNN1A, |
| | respiratory | digestive system, | those described in WO2015157070 |
| | system | reproductive system, | |
| | | exocrine, glands, | |
| Diabetic nephropathy | kidney | | Gremlin, 12/15-lipoxygenase, TIM44, |
| Dent Disease (Types 1 and 2) | Kidney | | Type 1: CLCN5, Type 2: ORCL |
| Dentatorubral-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; GRIA2; |
| | | | Grm5; Grin1; Htr1b; Grin2a; Drd3; |
| | | | Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various - | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholesterolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various - blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates - | |
| | | liver, brain, eyes | |
| Gastrointestinal Epithelial | | | CISH |
| Cancer, GI cancer | | | |
| Gaucher Disease (Types 1, 2, and | Fat metabolism | Various-liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulosclerosis | kidney | | CC chemokine ligand 2 |
| Glycogen Storage Diseases | Metabolism | | SLC2A2, GLUT2, G6PC, G6PT, |
| Types I-VI -See also Cori's | Diseases | | G6PT1, GAA, LAMP2, LAMPB, |
| Disease, Pompe Disease, | | | AGL, GDE, GBE1, GYS2, PYGL, |
| McArdle's disease, Hers Disease, | | | PFKM, see also Cori's Disease, |
| and Von Gierke's disease | | | Pompe's Disease, McArdle's disease, |
| | | | Hers Disease, and Von Gierke's |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup disease | Malabsorption | Various- brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various- | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB, FIX |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXIN1, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various- liver, | IDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various- liver, | IDUA, α-L-iduronidase |
| mucopolysaccharidosis type I H, | storage disease | spleen, eye, | |
| MPS IH) | | joint, heart, | |
| | | brain, skeletal | |
| Hurler-Scheie syndrome (aka | Lysosomal | Various- liver, | IDUA, α-L-iduronidase |
| mucopolysaccharidosis type I H- | storage disease | spleen, eye, | |
| S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g. galactosemia), Amino |
| organic acidemias), fatty acid | | | acid Metabolism disorders (e.g. |
| oxidation defects, amino | | | phenylketonuria), Fatty acid |
| acidopathies, carbohydrate | | | metabolism (e.g. MCAD deficiency), |
| disorders, mitochondrial | | | Urea Cycle disorders (e.g. |
| disorders | | | Citrullinemia), Organic acidemias |
| | | | (e.g. Maple Syrup Urine disease), |
| | | | Mitochondrial disorders (e.g. |
| | | | MELAS), peroxisomal disorders (e.g. |
| | | | Zellweger syndrome) |
| Inflammation | Various | | IL-10; IL-1 (IL-1a; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g., Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various - joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulo- nephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HTRA1, |
| | | | IL6, IL8, PLEKHA1, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinese | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease - | | Hurler disease, Sanfilippo syndrome, |
| C, IS, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDC1C, LGMD2I, |
| | | | TTN, CMD1G, TMD, LGMD2J, |
| | | | POMT1, CAV3, LGMD1C, SEPN1, |
| | | | SELN, RSMD1, PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; |
| | | | Notch1; Notch2; Notch3; Notch4; |
| | | | AKT; AKT2; AKT3; HIF; |
| | | | HIF1a; HIF3a; Met; HRG; Bcl2; |
| | | | PPAR alpha; PPAR |
| | | | gamma; WT1 (Wilms Tumor); FGF |
| | | | Receptor Family |
| | | | members (5 members: 1, 2, 3, 4, 5); |
| | | | CDKN2a; APC; RB |
| | | | (retinoblastoma); MEN1; VHL; |
| | | | BRCA1; BRCA2; AR |
| | | | (Androgen Receptor); TSG101; IGF; |
| | | | IGF Receptor; Igf1 (4 |
| | | | variants); Igf2 (3 variants); Igf 1 |
| | | | Receptor; Igf 2 Receptor; |
| | | | Bax; Bcl2; caspases family (9 |
| | | | members: |
| | | | 1, 2, 3, 4, 6, 7, 8, 9, 12); Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various- where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe Disease | Glycogen | Various - heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g. type | Various - eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCA1B, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROM1, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROM1, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, see also |
| | | | 20120204282 |
| Scheie syndrome (also known as | Various- liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor for |
| | | | Neuregulin); |
| | | | Complexin1 (Cplx1); Tph1 |
| | | | Tryptophan hydroxylase; Tph2 |
| | | | Tryptophan hydroxylase 2; Neurexin |
| | | | 1; GSK3; GSK3a; |
| | | | GSK3b; 5-HTT (Slc6a4); COMT; |
| | | | DRD (Drd1a); SLC6A3; DAOA; |
| | | | DTNBP1; Dao (Dao1); TCF4; |
| | | | COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR1I2, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLRE1C, ARTEMIS, |
| Immunodeficiency (SCID) and | | | SCIDA, RAG1, RAG2, ADA, PTPRC, |
| SCID-X1, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between −92 |
| | | | and −130 of the HBG Transcription |
| | | | Start Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | 1/2 promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at |
| syndrome) | | | a location known as 22q11.2, including |
| | | | TBX1, DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; |
| (generally) | | | FXN/X25 ATX3; |
| | | | ATXN1; ATXN2; |
| | | | DMPK; Atrophin-1 and Atn1 |
| | | | (DRPLA Dx); CBP (Creb-BP - global |
| | | | instability); VLDLR; Atxn7; Atxn10; |
| | | | FEN1, TNRC6A, PABPN1, JPH3, |
| | | | MED15, ATXN1, ATXN3, TBP, |
| | | | CACNA1A, ATXN80S, PPP2R2B, |
| | | | ATXN7, TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGR1, UNG, NUMBL, FABP2, |
| | | | EN2, CRYGC, SRP14, CRYGB, |
| | | | PDCD1, HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, see also |
| | | | 20110016540 |
| Turner's Syndrome (XO) | Various - | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various - | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler cardio-facial | | | |
| syndrome) | | | |
| Von Gierke's Disease (Glycogen | Glycogen | Various - liver, | G6PC and SLC37A4 |
| Storage Disease type I) | Storage disease | kidney | |
| Von Hippel-Lindau Syndrome | Various - cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various - | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In one embodiment, the compositions, systems, or components thereof can be used treat or prevent a disease in a subject by modifying one or more genes associated with one or more cellular functions, such as any one or more of those in Table 7. In one embodiment, the disease is a genetic disease or disorder. In some of embodiments, the composition, system, or component thereof can modify one or more genes or polynucleotides associated with one or more genetic diseases such as any set forth in Table 7.

TABLE 7
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; PTEN; EIF4E; |
| | PRKCZ; GRK6; MAPK1; TSC1; PLK1; AKT2; IKBKB; PIK3CA; CDK8; |
| | CDKN1B; NFKB2; BCL2; PIK3CB; PPP2R1A; MAPK8; BCL2L1; MAPK3; |
| | TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKC1; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid | RAC1; TAF4B; EP300; SMAD2; TRAF6; PCAF; ELK1; |
| Receptor | MAPK1; SMAD3; AKT2; IKBKB; NCOR2; UBE2I; |
| Signaling | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal | PRKCE; ITGAM; ROCK1; ITGA5; CXCR4; ADAM12; |
| Guidance | IGF1; RAC1; RAP1A; EIF4E; PRKCZ; NRP1; NTRK2; |
| Signaling | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor | PRKCE; ITGAM; ROCK1; ITGA5; CXCR4; IRAK1; |
| Signaling Actin | PRKAA2; EIF2AK2; RAC1; RAP1A; GRK6; ROCK2; |
| Cytoskeleton | MAPK1; PGF; RAC2; PTPN11; GNAS; PLK1; AKT2; |
| Signaling | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's | PRKCE; IGF1; EP300; RCOR1; PRKCZ; HDAC4; TGM2; |
| Disease | MAPK1; CAPNS1; AKT2; EGFR; NCOR2; SP1; CAPN2; |
| Signaling | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis | PRKCE; ROCK1; BID; IRAK1; PRKAA2; EIF2AK2; BAK1; |
| Signaling | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor | RAC1; PTEN; LYN; ELK1; MAPK1; RAC2; PTPN11; |
| Signaling | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte | ACTN4; CD44; PRKCE; ITGAM; ROCK1; CXCR4; CYBA; |
| Extravasation | RAC1; RAP1A; PRKCZ; ROCK2; RAC2; PTPN11; |
| Signaling | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase | IRAK1; SOD2; MYD88; TRAF6; ELK1; MAPK1; PTPN11; |
| Response | AKT2; IKBKB; PIK3CA; FOS; NFKB2; MAP3K14; |
| Signaling | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| Aryl Hydrocarbon | BIRC5; AKT2; PIK3CA; CHEK1; TP53INP1; BCL2; |
| Receptor Signaling | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| Wnt & Beta catenin | MAPK1; PTPN11; AKT2; EGFR; ERBB2; PRKCI; |
| Signaling | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor | PTEN; INS; EIF4E; PTPN1; PRKCZ; MAPK1; TSC1; |
| Signaling | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated | PRKCE; EP300; SOD2; PRKCZ; MAPK1; SQSTM1; |
| Oxidative | NQO1; PIK3CA; PRKCI; FOS; PIK3CB; PIK3C3; MAPK8; |
| Stress Response | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/ | EDN1; IGF1; KDR; FLT1; SMAD2; FGFR1; MET; PGF; |
| Hepatic Stellate | SMAD3; EGFR; FAS; CSF1; NFKB2; BCL2; MYH9; |
| Cell Activation | IGF1R; IL6R; RELA; TLR4; PDGFRB; TNF; RELB; IL8; |
| | PDGFRA; NFKB1; TGFBR1; SMAD4; VEGFA; BAX; |
| | ILIR1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI | PRKCE; RAC1; PRKCZ; LYN; MAPK1; RAC2; PTPN11; |
| Signaling | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer | PRKCE; RAC1; PRKCZ; MAPK1; RAC2; PTPN11; |
| Cell Signaling | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor | RAC1; ELK1; MAPK1; IKBKB; CBL; PIK3CA; FOS; |
| Signaling | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor | CRADD; HSPB1; BID; BIRC4; TBK1; IKBKB; FADD; |
| Signaling | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and | PRKCE; IRAK1; PRKAA2; EIF2AK2; GRK6; MAPK1; |
| Nicotinamide | PLK1; AKT2; CDK8; MAPK8; MAPK3; PRKCD; PRKAA1; |
| Metabolism | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARM1; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor | IRAK1; EIF2AK2; MYD88; TRAF6; PPARA; ELK1; |
| Signaling | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK | NTRK2; MAPK1; PTPN11; PIK3CA; CREB1; FOS; |
| Signaling | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated | IRAK1; MYD88; TRAF6; PPARA; RXRA; ABCA1; |
| Inhibition of | MAPK8; ALDH1A1; GSTP1; MAPK9; ABCB1; TRAF2; |
| RXR Function | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide | KDR; FLT1; PGF; AKT2; PIK3CA; PIK3CB; PIK3C3; |
| Signaling in the | CAV1; PRKCD; NOS3; PIK3C2A; AKT1; PIK3R1; |
| Cardiovascular System | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLD1; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial | SOD2; MAPK8; CASP8; MAPK10; MAPK9; CASP9; |
| Dysfunction | PARK7; PSEN1; PARK2; APP; CASP3 |
| Notch Signaling | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

In an aspect, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the particle delivery system or the delivery system or the virus particle of any one of the above embodiment or the cell of any one of the above embodiment, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In one embodiment, the composition, system(s), or component(s) thereof can be used to diagnose, prognose, treat, and/or prevent an infectious disease caused by a microorganism, such as bacteria, virus, fungi, parasites, or combinations thereof.

In one embodiment, the system(s) or component(s) thereof can be capable of targeting specific microorganism within a mixed population. Exemplary methods of such techniques are described in e.g., Gomaa A A, Klumpe H E, Luo M L, Selle K, Barrangou R, Beisel C L. 2014. Programmable removal of bacterial strains by use of genome-targeting composition, systems, mBio 5:e00928-13; Citorik R J, Mimee M, Lu T K. 2014. Sequence-specific antimicrobials using efficiently delivered RNA-guided nucleases. Nat Biotechnol 32:1141-1145, the teachings of which can be adapted for use with the compositions, systems, and components thereof described herein.

In one embodiment, the composition, system(s), and/or components thereof can be capable of targeting pathogenic and/or drug-resistant microorganisms, such as bacteria, virus, parasites, and fungi. In one embodiment, the composition, system(s), and/or components thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In one embodiment, the pathogenic bacteria that can be targeted and/or modified by the composition, system(s), and/or component(s) thereof described herein include, but are not limited to, those of the genus Actinomyces (e.g. A. israelii), Bacillus (e.g. B. anthracis, B. cereus), Bactereoides (e.g. B. fragilis), Bartonella (B. henselae, B. quintana), Bordetella (B. pertussis), Borrelia (e.g. B. burgdorferi, B. garinii, B. afzelii, and B. recurreentis), Brucella (e.g. B. abortus, B. canis, B. melitensis, and B. suis), Campylobacter (e.g. C. jejuni), Chlamydia (e.g. C. pneumoniae and C. trachomatis), Chlamydophila (e.g. C. psittaci), Clostridium (e.g. C. botulinum, C. difficile, C. perfringens. C. tetani), Corynebacterium (e.g. C. diptheriae), Enterococcus (e.g. E. Faecalis, E. faecium), Ehrlichia (E. canis and E. chaffensis) Escherichia (e.g. E. coli), Francisella (e.g. F. tularensis), Haemophilus (e.g. H. influenzae), Helicobacter (H. pylori), Klebsiella (E.g. K. pneumoniae), Legionella (e.g. L. pneumophila), Leptospira (e.g. L. interrogans, L. santarosai, L. weilii, L. noguchii), Listereia (e.g. L. monocytogeenes), Mycobacterium (e.g. M. leprae, M. tuberculosis, M. ulcerans), Mycoplasma (M. pneumoniae), Neisseria (N. gonorrhoeae and N. menigitidis), Nocardia (e.g. N. asteroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streeptococcus (S. agalactiaee, S. pneumoniae, S. pyogenes), Treponema (T. pallidum), Ureeaplasma (e.g. U. urealyticum), Vibrio (e.g. V. cholerae), Yersinia (e.g. Y. pestis, Y. enteerocolitica, and Y. pseudotuberculosis).

In one embodiment, the pathogenic virus that can be targeted and/or modified by the composition, system(s), and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In one embodiment, the pathogenic virus can be from the family Adenoviridae (e.g., Adenovirus), Herpeesviridae (e.g., Herpes simplex, type 1, Herpes simplex, type 2, Varicella-zoster virus, Epstein-Barr virus, Human cytomegalovirus, Human herpesvirus, type 8), Papillomaviridae (e.g., Human papillomavirus), Polyomaviridae (e.g., BK virus, JC virus), Poxviridae (e.g., smallpox), Hepadnaviridae (e.g., Hepatitis B), Parvoviridae (e.g., Parvovirus B19), Astroviridae (e.g., Human astrovirus), Caliciviridae (e.g., Norwalk virus), Picornaviridae (e.g., coxsackievirus, hepatitis A virus, poliovirus, rhinovirus), Coronaviridae (e.g., Severe acute respiratory syndrome-related coronavirus, strains: Severe acute respiratory syndrome virus, Severe acute respiratory syndrome coronavirus 2 (COVID-19)), Flaviviridae (e.g., Hepatitis C virus, yellow fever virus, dengue virus, West Nile virus, TBE virus), Togaviridae (e.g., Rubella virus), Hepeviridae (e.g., Hepatitis E virus), Retroviridae (Human immunodeficiency virus (HIV)), Orthomyxoviridae (e.g., Influenza virus), Arenaviridae (e.g., Lassa virus), Bunyaviridae (e.g., Crimean-Congo hemorrhagic fever virus, Hantaan virus), Filoviridae (e.g., Ebola virus and Marburg virus), Paramyxoviridae (e.g., Measles virus, Mumps virus, Parainfluenza virus, Respiratory syncytial virus), Rhabdoviridae (Rabies virus), Hepatits D virus, Reoviridae (e.g., Rotavirus, Orbivirus, Coltivirus, Banna virus).

In one embodiment, the pathogenic fungi that can be targeted and/or modified by the composition, system(s), and/or component(s) thereof described herein include, but are not limited to, those of the genus Candida (e.g., C. albicans), Aspergillus (e.g., A. fumigatus, A. flavus, A. clavatus), Cryptococcus (e.g., C. neoformans, C. gattii), Histoplasma (H. capsulatum), Pneumocystis (e.g., P. jiroveecii), Stachybotrys (e.g., S. chartarum).

In one embodiment, the pathogenic parasites that can be targeted and/or modified by the composition, system(s), and/or component(s) thereof described herein include, but are not limited to, protozoa, helminths, and ectoparasites. In one embodiment, the pathogenic protozoa that can be targeted and/or modified by the composition, system(s), and/or component(s) thereof described herein include, but are not limited to, those from the groups Sarcodina (e.g., ameba such as Entamoeba), Mastigophora (e.g., flagellates such as Giardia and Leishmania), Cilophora (e.g., ciliates such as Balantidum), and sporozoa (e.g., plasmodium and cryptosporidium). In one embodiment, the pathogenic helminths that can be targeted and/or modified by the composition, system(s), and/or component(s) thereof described herein include, but are not limited to, flatworms (platyhelminths), thorny-headed worms (acanthoceephalins), and roundworms (nematodes). In one embodiment, the pathogenic ectoparasites that can be targeted and/or modified by the composition, system(s), and/or component(s) thereof described herein include, but are not limited to, ticks, fleas, lice, and mites.

In one embodiment, the pathogenic parasite that can be targeted and/or modified by the composition, system(s), and/or component(s) thereof described herein include, but are not limited to, Acanthamoeba spp., Balamuthia mandrillaris, Babesiosis spp. (e.g., Babesia B. divergens, B. bigemina, B. equi, B. microfti, B. duncani), Balantidiasis spp. (e.g., Balantidium coli), Blastocystis spp., Cryptosporidium spp., Cyclosporiasis spp. (e.g., Cyclospora cayetanensis), Dientamoebiasis spp. (e.g., Dientamoeba fragilis), Amoebiasis spp. (e.g., Entamoeba histolytica), Giardiasis spp. (e.g., Giardia lamblia), Isosporiasis spp. (e.g., Isospora belli), Leishmania spp., Naegleria spp. (e.g., Naegleria fowleri), Plasmodium spp. (e.g., Plasmodium falciparum, Plasmodium vivax, Plasmodium ovale curtisi, Plasmodium ovale wallikeri, Plasmodium malariae, Plasmodium knowlesi), Rhinosporidiosis spp. (e.g., Rhinosporidium seeberi), Sarcocystosis spp. (e.g., Sarcocystis bovihominis, Sarcocystis suihominis), Toxoplasma spp. (e.g., Toxoplasma gondii), Trichomonas spp. (e.g., Trichomonas vaginalis), Trypanosoma spp. (e.g., Trypanosoma brucei), Trypanosoma spp. (e.g., Trypanosoma cruzi), Tapeworm (e.g., Cestoda, Taenia multiceps, Taenia saginata, Taenia solium), Diphyllobothrium latum spp., Echinococcus spp. (e.g., Echinococcus granulosus, Echinococcus multilocularis, E. vogeli, E. oligarthrus), Hymenolepis spp. (e.g., Hymenolepis nana, Hymenolepis diminuta), Bertiella spp. (e.g., Bertiella mucronata, Bertiella studeri), Spirometra (e.g., Spirometra erinaceieuropaei), Clonorchis spp. (e.g., Clonorchis sinensis; Clonorchis viverrini), Dicrocoelium spp. (e.g., Dicrocoelium dendriticum), Fasciola spp. (e.g., Fasciola hepatica, Fasciola gigantica), Fasciolopsis spp. (e.g., Fasciolopsis buski), Metagonimus spp. (e.g., Metagonimus yokogawai), Metorchis spp. (e.g., Metorchis conjunctus), Opisthorchis spp. (e.g., Opisthorchis viverrini, Opisthorchis felineus), Clonorchis spp. (e.g., Clonorchis sinensis), Paragonimus spp. (e.g., Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g., Schistosoma mansoni, Schistosoma haematobium, Schistosoma japonicum, Schistosoma mekongi, and Schistosoma intercalatum), Echinostoma spp. (e.g., E. echinatum), Trichobilharzia spp. (e.g., Trichobilharzia regent), Ancylostoma spp. (e.g., Ancylostoma duodenale), Necator spp. (e.g., Necator americanus), Angiostrongylus spp., Anisakis spp., Ascaris spp. (e.g., Ascaris lumbricoides), Baylisascaris spp. (e.g., Baylisascaris procyonis), Brugia spp. (e.g., Brugia malayi, Brugia timori), Dioctophyme spp. (e.g., Dioctophyme renale), Dracunculus spp. (e.g., Dracunculus medinensis), Enterobius spp. (e.g., Enterobius vermicularis, Enterobius gregorii), Gnathostoma spp. (e.g., Gnathostoma spinigerum, Gnathostoma hispidum), Halicephalobus spp. (e.g., Halicephalobus gingivalis), Loa loa spp. (e.g., Loa loa filaria), Mansonella spp. (e.g., Mansonella streptocerca), Onchocerca spp. (e.g., Onchocerca volvulus), Strongyloides spp. (e.g., Strongyloides stercoralis), Thelazia spp. (e.g., Thelazia californiensis, Thelazia callipaeda), Toxocara spp. (e.g., Toxocara canis, Toxocara cati, Toxascaris leonine), Trichinella spp. (e.g., Trichinella spiralis, Trichinella britovi, Trichinella nelsoni, Trichinella nativa), Trichuris spp. (e.g., Trichuris trichiura, Trichuris vulpis), Wuchereria spp. (e.g., Wuchereria bancrofti), Dermatobia spp. (e.g., Dermatobia hominis), Tunga spp. (e.g., Tunga penetrans), Cochliomyia spp. (e.g., Cochliomyia hominivorax), Linguatula spp. (e.g., Linguatula serrata), Archiacanthocephala sp., Moniliformis sp. (e.g., Moniliformis moniliformis), Pediculus spp. (e.g. Pediculus humanus capitis, Pediculus humanus humanus), Pthirus spp. (e.g., Pthirus pubis), Arachnida spp. (e.g., Trombiculidae, Ixodidae, Argaside), Siphonaptera spp (e.g., Siphonaptera: Pulicinae), Cimicidae spp. (e.g., Cimex lectularius and Cimex hemipterus), Diptera spp., Demodex spp. (e.g., Demodex folliculorum/brevis/canis), Sarcoptes spp. (e.g., Sarcoptes scabiei), Dermanyssus spp. (e.g., Dermanyssus gallinae), Ornithonyssus spp. (e.g., Ornithonyssus sylviarum, Ornithonyssus bursa, Ornithonyssus bacoti), Laelaps spp. (e.g., Laelaps echidnina), Liponyssoides spp. (e.g., Liponyssoides sanguineus).

In one embodiment, the gene targets can be any of those as set forth in Table 1 of Strich and Chertow. 2019. J. Clin. Microbio. 57:4 e01307-18, which is incorporated herein as if expressed in its entirety herein.

In one embodiment, the method can include delivering a composition, system, and/or component thereof to a pathogenic organism described herein, allowing the composition, system, and/or component thereof to specifically bind and modify one or more targets in the pathogenic organism, whereby the modification kills, inhibits, reduces the pathogenicity of the pathogenic organism, or otherwise renders the pathogenic organism non-pathogenic. In one embodiment, delivery of the composition, system, occurs in vivo (i.e., in the subject being treated). In one embodiment occurs by an intermediary, such as microorganism or phage that is non-pathogenic to the subject but is capable of transferring polynucleotides and/or infecting the pathogenic microorganism. In one embodiment, the intermediary microorganism can be an engineered bacteria, virus, or phage that contains the composition, system(s), and/or component(s) thereof and/or vectors and/or vector systems. The method can include administering an intermediary microorganism containing the composition, system(s), and/or component(s) thereof and/or vectors and/or vector systems to the subject to be treated. The intermediary microorganism can then produce the compositions and/or component thereof or transfer a composition, system, polynucleotide to the pathogenic organism. In embodiments, where the compositions and/or component thereof, vector, or vector system is transferred to the pathogenic microorganism, the composition, system, or component thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In one embodiment, where the pathogenic microorganism inserts its genetic material into the host cell's genome (e.g., a virus), the composition, system, can be designed such that it modifies the host cell's genome such that the viral DNA or cDNA cannot be replicated by the host cell's machinery into a functional virus. In one embodiment, where the pathogenic microorganism inserts its genetic material into the host cell's genome (e.g., a virus), the composition, system, can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In one embodiment, mtDNA mutations can be modified using a composition, system, described herein. In one embodiment, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, or a combination thereof.

In one embodiment, the mtDNA of a subject can be modified in vivo or ex vivo. In one embodiment, where the mtDNA is modified ex vivo, after modification the cells containing the modified mitochondria can be administered back to the subject. In one embodiment, the composition, system, or component thereof can be capable of correcting an mtDNA mutation, or a combination thereof.

In one embodiment, at least one of the one or more mtDNA mutations is selected from the group consisting of: A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem (SEQ ID NO: 53) repeats at positions 305-314 and/or 956-965, deletion at positions from 8,469-13,447, 4,308-14,874, and/or 4,398-14,822, 961ins/delC, the mitochondrial common deletion (e.g., mtDNA 4,977 bp deletion), and combinations thereof.

In one embodiment, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In one embodiment, the method includes delivering a composition, system, and/or a component thereof to a cell, and more specifically one or more mitochondria in a cell, allowing the composition, system, and/or component thereof to modify one or more target polynucleotides in the cell, and more specifically one or more mitochondria in the cell. The target polynucleotides can correspond to a mutation in the mtDNA, such as any one or more of those described herein. In one embodiment, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

### Microbiome Modification

Microbiomes play important roles in health and disease. For example, the gut microbiome can play a role in health by controlling digestion, preventing growth of pathogenic microorganisms and have been suggested to influence mood and emotion. Imbalanced microbiomes can promote disease and are suggested to contribute to weight gain, unregulated blood sugar, high cholesterol, cancer, and other disorders. A healthy microbiome has a series of joint characteristics that can be distinguished from non-healthy individuals, thus detection and identification of the disease-associated microbiome can be used to diagnose and detect disease in an individual. The compositions, systems, and components thereof can be used to screen the microbiome cell population and be used to identify a disease associated microbiome. Cell screening methods utilizing compositions, systems, and components thereof are described elsewhere herein and can be applied to screening a microbiome, such as a gut, skin, vagina, and/or oral microbiome, of a subject.

In one embodiment, the microbe population of a microbiome in a subject can be modified using a composition, system, and/or component thereof described herein. In one embodiment, the composition, system, and/or component thereof can be used to identify and select one or more cell types in the microbiome and remove them from the microbiome population. Exemplary methods of selecting cells using a composition, system, and/or component thereof are described elsewhere herein. In this way the make-up or microorganism profile of the microbiome can be altered. In one embodiment, the alteration causes a change from a diseased microbiome composition to a healthy microbiome composition. In this way the ratio of one type or species of microorganism to another can be modified, such as going from a diseased ratio to a healthy ratio. In one embodiment, the cells selected are pathogenic microorganisms.

In one embodiment, the compositions and systems described herein can be used to modify a polynucleotide in a microorganism of a microbiome in a subject. In one embodiment, the microorganism is a pathogenic microorganism. In one embodiment, the microorganism is a commensal and non-pathogenic microorganism. Methods of modifying polynucleotides in a cell in the subject are described elsewhere herein and can be applied to these embodiments.

### Models of Diseases and Conditions

In an aspect, the invention provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiment.

In one aspect, the invention provides a method of generating a model eukaryotic cell that can include one or more a mutated disease genes and/or infectious microorganisms. In one embodiment, a disease gene is any gene associated an increase in the risk of having or developing a disease. In one embodiment, the method includes (a) introducing one or more vectors into a eukaryotic cell, wherein the one or more vectors comprise a composition, system, and/or component thereof and/or a vector or vector system that is capable of driving expression of a composition, system, and/or component thereof including, but not limited to: a guide sequence, one or more Cas5-HNH or Cas8-HNH polypeptide nucleases, and combinations thereof and (b) allowing a composition, system, or complex to bind to one or more target polynucleotides, e.g., to effect cleavage, nicking, or other modification of the target polynucleotide within said disease gene, wherein the composition, system, or complex is composed of one or more Cas5-HNH or Cas8-HNH polypeptide nuclease complexed with (1) one or more guide sequences that is/are hybridized to the target sequence(s) within the target polynucleotide(s), and optionally (2) the guide RNA scaffold sequence(s), thereby generating a model eukaryotic cell comprising one or more mutated disease gene(s). Thus, in one embodiment the composition and system, contains nucleic acid molecules for and drives expression of one or more of: a Cas5-HNH or Cas8-HNH polypeptide nuclease, a guide sequence and/or a Homologous Recombination template and/or a stabilizing ligand if the Cas5-HNH or Cas8-HNH polypeptide nuclease has a destabilization domain. In one embodiment, said cleavage comprises cleaving one or two strands at the location of the target sequence by the Cas5-HNH or Cas8-HNH polypeptide nuclease. In one embodiment, nicking comprises nicking one or two strands at the location of the target sequence by the Cas5-HNH or Cas8-HNH polypeptide nuclease. In one embodiment, said cleavage or nicking results in modified transcription of a target polynucleotide. In one embodiment, modification results in decreased transcription of the target polynucleotide. In one embodiment, the method further comprises repairing said cleaved or nicked target polynucleotide by homologous recombination with a recombination template polynucleotide, wherein said repair results in a mutation comprising an insertion, deletion, or substitution of one or more nucleotides of said target polynucleotide. In one embodiment, said mutation results in one or more amino acid changes in a protein expression from a gene comprising the target sequence.

The disease modeled can be any disease with a genetic or epigenetic component. In one embodiment, the disease modeled can be any as discussed elsewhere herein.

### In Situ Disease Detection

The compositions, systems, and/or components thereof can be used for diagnostic methods of detection such as in CASFISH (see e.g., Deng et al. 2015. PNAS USA 112 (38): 11870-11875), CRISPR-Live FISH (see e.g., Wang et al. 2020. Science; 365 (6459): 1301-1305), sm-FISH (Lee and Jefcoate. 2017. Front. Endocrinol. doi.org/10.3389/fendo.2017.00289), sequential FISH CRISPRainbow (Ma et al. Nat Biotechnol, 34 (2016), pp. 528-530), CRISPR-Sirius (Nat Methods, 15 (2018), pp. 928-931), Casilio (Cheng et al. Cell Res, 26 (2016), pp. 254-257), Halo-Tag based genomic loci visualization techniques (e.g., Deng et al. 2015. PNAS USA 112 (38): 11870-11875; Knight et al., Science, 350 (2015), pp. 823-826), RNA-aptamer based methods (e.g. Ma et al., J Cell Biol, 214 (2016), pp. 529-537), molecular beacon-based methods (e.g. Zhao et al. Biomaterials, 100 (2016), pp. 172-183; Wu et al. Nucleic Acids Res (2018)), Quantum Dot-based systems (e.g. Ma et al. Anal Chem, 89 (2017), pp. 12896-12901), multiplexed methods (e.g. Ma et al., Proc Natl Acad Sci USA, 112 (2015), pp. 3002-3007; Fu et al. Nat Commun, 7 (2016), p. 11707; Ma et al. Nat Biotechnol, 34 (2016), pp. 528-530; Shao et al. Nucleic Acids Res, 44 (2016), Article e86); Wang et al. Sci Rep, 6 (2016), p. 26857), ç, and other in situ CRISPR-hybridization based methods (e.g. Chen et al. Cell, 155 (2013), pp. 1479-1491; Gu et al. Science, 359 (2018), pp. 1050-1055; Tanebaum et al. Cell, 159 (2014), pp. 635-646; Ye et al. Protein Cell, 8 (2017), pp. 853-855; Chen et al. Nat Commun, 9 (2018), p. 5065; Shao et al. ACS Synth Biol (2017); Fu et al. Nat Commun, 7 (2016), p. 11707; Shao et al. Nucleic Acids Res, 44 (2016), Article e86; Wang et al., Sci Rep, 6 (2016), p. 26857), all of which are incorporated by reference herein as if expressed in their entirety and whose teachings can be adapted to the compositions, systems, and components thereof described herein in view of the description herein.

In one embodiment, the composition, system, or component thereof can be used in a detection method, such as an in-situ detection method described herein. In one embodiment, the composition, system, or component thereof can include a catalytically inactivate Cas5-HNH or Cas8-HINH polypeptide nuclease described herein and use this system in detection methods such as fluorescence in situ hybridization (FISH) or any other described herein. In one embodiment, the inactivated Cas5-HNH or Cas8-HNH polypeptide nuclease, which lacks the ability to produce DNA double-strand breaks may be fused with a marker, such as fluorescent protein, such as the enhanced green fluorescent protein (eEGFP) and co-expressed with small guide RNAs to target pericentric, centric and telomeric repeats in vivo. The dead Cas5-HNH or Cas8-HNH polypeptide nuclease or system thereof can be used to visualize both repetitive sequences and individual genes in the human genome. Such new applications of labelled dead Cas5-HNH or Cas8-HNH polypeptide nuclease and compositions, systems, thereof can be important in imaging cells and studying the functional nuclear architecture, especially in cases with a small nucleus volume or complex 3-D structures.

### Cell Selection

In one embodiment, the compositions, systems, and/or components thereof described herein can be used in a method to screen and/or select cells. In one embodiment, system-based screening/selection method can be used to identify diseased cells in a cell population. In one embodiment, selection of the cells results in a modification in the cells such that the selected cells die. In this way, diseased cells can be identified, and removed from the healthy cell population. In one embodiment, the diseased cells can be a cancer cell, pre-cancerous cell, a virus or other pathogenic organism infected cells, or otherwise abnormal cell. In one embodiment, the modification can impart another detectable change in the cells to be selected (e.g., a functional change and/or genomic barcode) that facilitates selection of the desired cells. In one embodiment, a negative selection scheme can be used to obtain a desired cell population. In these embodiments, the cells to be selected against are modified, thus can be removed from the cell population based on their death or identification or sorting based the detectable change imparted on the cells. Thus, in these embodiments, the remaining cells after selection are the desired cell population.

In one embodiment, a method of selecting one or more cell(s) containing a polynucleotide modification can include: introducing one or more composition, system(s), and/or components thereof, and/or vectors or vector systems into the cell(s), wherein the composition, system(s), and/or components thereof, and/or vectors or vector systems contains and/or is capable of expressing one or more of: a Cas5-HNH or Cas8-HNH polypeptide nuclease, an guide RNA sequence, and an recombination template; wherein, for example that which is being expressed is within and expressed in vivo by the composition, system, vector or vector system and/or the recombination template comprises the one or more mutations that abolish Cas5-HNH or Cas8-HINH polypeptide nuclease cleavage; allowing homologous recombination of the recombination template with the target polynucleotide in the cell(s) to be selected; allowing a composition, system, or complex to bind to a target polynucleotide to effect cleavage of the target polynucleotide within said gene, wherein the AAV-complex comprises the Cas5-HNH or Cas8-HNH polypeptide nuclease complexed with (1) the guide sequence that is hybridized to the target sequence within the target polynucleotide, and (2) the guide RNA scaffold, wherein binding of the complex to the target polynucleotide induces cell death or imparts some other detectable change to the cell, thereby allowing one or more cell(s) in which one or more mutations have been introduced to be selected. In one embodiment, the cell to be selected may be a eukaryotic cell. In one embodiment, the cell to be selected may be a prokaryotic cell. Selection of specific cells via the methods herein can be performed without requiring a selection marker or a two-step process that may include a counter-selection system.

### Therapeutic Agent Development

The compositions, systems, and components thereof described herein can be used to develop Cas5-HNH or Cas8-HNH polypeptide nuclease-based biologically active agents, such as small molecule therapeutics. Thus, described herein are methods for developing a biologically active agent that modulates a cell function and/or signaling event associated with a disease and/or disease gene. In one embodiment, the method comprises (a) contacting a test compound with a diseased cell and/or a cell containing a disease gene cell; and (b) detecting a change in a readout that is indicative of a reduction or an augmentation of a cell signaling event or other cell functionality associated with said disease or disease gene, thereby developing said biologically active agent that modulates said cell signaling event or other functionality associated with said disease gene. In one embodiment, the diseased cell is a model cell described elsewhere herein. In one embodiment, the diseased cell is a diseased cell isolated from a subject in need of treatment. In one embodiment, the test compound is a small molecule agent. In one embodiment, test compound is a small molecule agent. In one embodiment, the test compound is a biologic molecule agent.

In one embodiment, the method involves developing a therapeutic based on the composition, system, described herein. In one embodiment, the therapeutic comprises a Cas5-HNH or Cas8-HNH polypeptide nuclease and/or a guide RNA with a reprogrammable spacer capable of hybridizing to a target sequence of interest. In one embodiment, the therapeutic is a vector or vector system that can contain a) a first regulatory element operably linked to a nucleotide sequence encoding the Cas5-HNH or Cas8-HNH polypeptide nuclease; and b) a second regulatory element operably linked to one or more nucleotide sequences encoding one or more nucleic acid molecules comprising a guide RNA comprising a reprogrammable spacer sequence, a conserved RNA sequence; wherein components (a) and (b) are located on same or different vectors. In one embodiment, the biologically active agent is a composition comprising a delivery system operably configured to deliver composition, system, or components thereof, and/or or one or more polynucleotide sequences, vectors, or vector systems containing or encoding said components into a cell and capable of forming a complex with the components of the composition and system herein, and wherein said complex is operable in the cell. In one embodiment, the complex can include the Cas5-HNH or Cas8-HNH polypeptide nuclease as described herein, guide RNA scaffold comprising the guide sequence (reprogrammable spacer sequence), and a conserved nucleotide sequence. In any such compositions, the delivery system can be a yeast system, a lipofection system, a microinjection system, a biolistic system, virosomes, liposomes, immunoliposomes, polycations, lipid: nucleic acid conjugates or artificial virions, or any other system as described herein. In one embodiment, the delivery is via a particle, a nanoparticle, a lipid or a cell penetrating peptide (CPP).

Also described herein are methods for developing or designing a composition, system,, optionally a composition, system, based therapy or therapeutic, comprising (a) selecting for a (therapeutic) locus of interest guide RNA target sites, wherein said target sites have minimal sequence variation across a population, and from said selected target sites sub-selecting target sites, wherein a guide RNA directed against said target sites recognizes a minimal number of off-target sites across said population, or (b) selecting for a (therapeutic) locus of interest guide RNA target sites, wherein said target sites have minimal sequence variation across a population, or selecting for a (therapeutic) locus of interest guide RNA target sites, wherein a guide RNA directed against said target sites recognizes a minimal number of off-target sites across said population, and optionally estimating the number of (sub) selected target sites needed to treat or otherwise modulate or manipulate a population, and optionally validating one or more of the (sub) selected target sites for an individual subject, optionally designing one or more guide RNA recognizing one or more of said (sub) selected target sites.

In one embodiment, the method for developing or designing a guide RNA for use in a composition, system, optionally a composition, system, based therapy or therapeutic, can include (a) selecting for a (therapeutic) locus of interest guide RNA target sites, wherein said target sites have minimal sequence variation across a population, and from said selected target sites sub-selecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more gRNA recognizing one or more of said (sub) selected target sites.

In one embodiment, thee method for developing or designing a composition, system, optionally a composition, system, based therapy or therapeutic in a population, can include (a) selecting for a (therapeutic) locus of interest reprogrammable spacer target sites, wherein said target sites have minimal sequence variation across a population, and from said selected target sites sub-selecting target sites, wherein a guide RNA directed against said target sites recognizes a minimal number of off-target sites across said population, or (b) selecting for a (therapeutic) locus of interest RNA reprogrammable spacer target sites, wherein said target sites have minimal sequence variation across a population, or selecting for a (therapeutic) locus of interest guide RNA reprogrammable spacer target sites, wherein a guide RNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more guide RNA recognizing one or more of said (sub) selected target sites.

In one embodiment the method for developing or designing a gRNA for use in a composition, system, optionally a composition, system, based therapy or therapeutic in a population, can include (a) selecting for a (therapeutic) locus of interest gRNA target sites, wherein said target sites have minimal sequence variation across a population, and from said selected target sites sub-selecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub) selected target sites needed to treat or otherwise modulate or manipulate a population, optionally validating one or more of the (sub) selected target sites for an individual subject, optionally designing one or more guide RNA reprogrammable spacer recognizing one or more of said (sub) selected target sites.

In one embodiment, the method for developing or designing a composition, system, such as a composition, system, based therapy or therapeutic, optionally in a population; or for developing or designing a guide RNA reprogrammable spacer for use in a composition, system, optionally a composition, system, based therapy or therapeutic, optionally in a population, can include selecting a set of target sequences for one or more loci in a target population, wherein the target sequences do not contain variants occurring above a threshold allele frequency in the target population (i.e., platinum target sequences); removing from said selected (platinum) target sequences any target sequences having high frequency off-target candidates (relative to other (platinum) targets in the set) to define a final target sequence set; preparing one or more, such as a set of compositions, systems, based on the final target sequence set, optionally wherein a number of composition prepared is based (at least in part) on the size of a target population.

In an embodiment, off-target candidates/off-targets, PAM restrictiveness, target cleavage efficiency, or effector protein specificity is identified or determined using a sequencing-based double-strand break (DSB) detection assay, such as described herein elsewhere. In an embodiment, off-target candidates/off-targets are identified or determined using a sequencing-based double-strand break (DSB) detection assay, such as described herein elsewhere. In an embodiment, off-targets, or off target candidates, have at least 1, preferably 1-3, mismatches or (distal) PAM mismatches, such as 1 or more, such as 1, 2, 3, or more, (distal) PAM mismatches. In an embodiment, sequencing-based DSB detection assay comprises labeling a site of a DSB with an adapter comprising a primer binding site, labeling a site of a DSB with a barcode or unique molecular identifier, or combination thereof, as described herein elsewhere.

It will be understood that the reprogrammable spacer sequence of the guide RNA is 100% complementary to the target site, i.e., does not comprise any mismatch with the target site. It will be further understood that “recognition” of an (off-) target site by a reprogrammable spacer presupposes composition, system, functionality, i.e. an (off-) target site is only recognized by a reprogrammable spacer RNA if binding of the reprogrammable spacer RNA to the (off-) target site leads to composition, system, activity (such as induction of single or double strand DNA cleavage, transcriptional modulation, etc.).

In an embodiment, the target sites having minimal sequence variation across a population are characterized by absence of sequence variation in at least 99%, preferably at least 99.9%, more preferably at least 99.99% of the population. In an embodiment, optimizing target location comprises selecting target sequences or loci having an absence of sequence variation in at least 99%, %, preferably at least 99.9%, more preferably at least 99.99% of a population. These targets are referred to herein elsewhere also as “platinum targets”. In an embodiment, said population comprises at least 1000 individuals, such as at least 5000 individuals, such as at least 10000 individuals, such as at least 50000 individuals.

In an embodiment, the off-target sites are characterized by at least one mismatch between the off-target site and the guide RNA. In an embodiment, the off-target sites are characterized by at most five, preferably at most four, more preferably at most three mismatches between the off-target site and the guide RNA. In an embodiment, the off-target sites are characterized by at least one mismatch between the off-target site and the guide RNA and by at most five, preferably at most four, more preferably at most three mismatches between the off-target site and the guide RNA.

In an embodiment, said minimal number of off-target sites across said population is determined for high-frequency haplotypes in said population. In an embodiment, said minimal number of off-target sites across said population is determined for high-frequency haplotypes of the off-target site locus in said population. In an embodiment, said minimal number of off-target sites across said population is determined for high-frequency haplotypes of the target site locus in said population. In an embodiment, the high-frequency haplotypes are characterized by occurrence in at least 0.1% of the population.

In an embodiment, the number of (sub) selected target sites needed to treat a population is estimated based on based low frequency sequence variation, such as low frequency sequence variation captured in large scale sequencing datasets. In an embodiment, the number of (sub) selected target sites needed to treat a population of a given size is estimated.

In an embodiment, the method further comprises obtaining genome sequencing data of a subject to be treated; and treating the subject with a composition, system, selected from the set of compositions, systems, wherein the composition, system, selected is based (at least in part) on the genome sequencing data of the individual. In an embodiment, the ((sub) selected) target is validated by genome sequencing, preferably whole genome sequencing.

In an embodiment, target sequences or loci as described herein are (further) selected based on optimization of one or more parameters, such as PAM type (natural or modified), PAM nucleotide content, PAM length, target sequence length, PAM restrictiveness, target cleavage efficiency, and target sequence position within a gene, a locus or other genomic region. Methods of optimization are discussed in greater detail elsewhere herein.

In an embodiment, target sequences or loci as described herein are (further) selected based on optimization of one or more of target loci location, target length, target specificity, and PAM characteristics. As used herein, PAM characteristics may comprise for instance PAM sequence, PAM length, and/or PAM GC contents. In an embodiment, optimizing PAM characteristics comprises optimizing nucleotide content of a PAM. In an embodiment, optimizing nucleotide content of PAM is selecting a PAM with a motif that maximizes abundance in the one or more target loci, minimizes mutation frequency, or both. Minimizing mutation frequency can for instance be achieved by selecting PAM sequences devoid of or having low or minimal CpG.

In an embodiment, the effector protein for each composition and system, in the set of compositions, systems, is selected based on optimization of one or more parameters selected from the group consisting of: effector protein size, ability of effector protein to access regions of high chromatin accessibility, degree of uniform enzyme activity across genomic targets, epigenetic tolerance, mismatch/budge tolerance, effector protein specificity, effector protein stability or half-life, effector protein immunogenicity or toxicity. Methods of optimization are discussed in greater detail elsewhere herein.

### Optimization of the Systems

The methods of the present invention can involve optimization of selected parameters or variables associated with the composition, system, and/or its functionality, as described herein further elsewhere. Optimization of the composition, system, in the methods as described herein may depend on the target(s), such as the therapeutic target or therapeutic targets, the mode or type of composition, system, modulation, such as composition, system, based therapeutic target(s) modulation, modification, or manipulation, as well as the delivery of the composition, system, components. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple guide RNAs and multiple reprogrammable spacers.

The activity of the composition and/or system, such as Cas5-HNH or Cas8-HNH polypeptide nuclease-based therapy or therapeutics may involve target disruption, such as target mutation, such as leading to gene knockout. The activity of the composition and/or system, such as Cas5-HNH or Cas8-HNH polypeptide nuclease-based therapy or therapeutics may involve replacement of particular target sites, such as leading to target correction. Cas5-HNH or Cas8-HNH polypeptide nuclease-based therapy or therapeutics may involve removal of particular target sites, such as leading to target deletion. The activity of the composition and/or system, such as Cas5-HNH or Cas8-HNH polypeptide nuclease-based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve Cas5-HNH or Cas8-HNH polypeptide nuclease mutation (such as for instance generation of a catalytically inactive Cas5-HNH or Cas8-HNH polypeptide nuclease) and/or functionalization (such as for instance fusion of the Cas5-HNH or Cas8-HNH polypeptide nuclease with a heterologous functional domain, such as a transcriptional activator or repressor), as described herein elsewhere.

Accordingly, in an aspect, the invention relates to a method as described herein, comprising selection of one or more (therapeutic) target, selecting one or more functionality of the composition and/or system, and optimization of selected parameters or variables associated with the composition and/or its functionality. In a related aspect, the invention relates to a method as described herein, comprising (a) selecting one or more (therapeutic) target loci, (b) selecting one or more composition functionalities, (c) optionally selecting one or more modes of delivery, and preparing, developing, or designing a composition herein selected based on steps (a)-(c).

In an embodiment, the functionality of the composition and/or system comprises genomic mutation. In an embodiment, the functionality of the composition and/or system comprises single genomic mutation. In an embodiment, the functionality of the composition and/or system functionality comprises multiple genomic mutation. In an embodiment, the functionality of the composition and/or system comprises gene knockout. In an embodiment, the functionality of the composition and/or system comprises single gene knockout. In an embodiment, the functionality of the composition and/or system comprises multiple gene knockout. In an embodiment, the functionality of the composition and/or system comprises gene correction. In an embodiment, the functionality of the composition and/or system comprises single gene correction. In an embodiment, the functionality of the composition and/or system comprises multiple gene correction. In an embodiment, the functionality of the composition and/or system comprises genomic region correction. In an embodiment, the functionality of the composition and/or system comprises single genomic region correction. In an embodiment, the functionality of the composition and/or system comprises multiple genomic region correction. In an embodiment, the functionality of the composition and/or system comprises gene deletion. In an embodiment, the functionality of the composition and/or system comprises single gene deletion. In an embodiment, the functionality of the composition and/or system comprises multiple gene deletion. In an embodiment, the functionality of the composition and/or system comprises genomic region deletion. In an embodiment, the functionality of the composition and/or system comprises single genomic region deletion. In an embodiment, the functionality of the composition and/or system comprises multiple genomic region deletion. In an embodiment, the functionality of the composition and/or system comprises modulation of gene or genomic region functionality. In an embodiment, the functionality of the composition and/or system comprises modulation of single gene or genomic region functionality. In an embodiment, the functionality of the composition and/or system comprises modulation of multiple gene or genomic region functionality. In an embodiment, the functionality of the composition and/or system comprises gene or genomic region functionality, such as gene or genomic region activity. In an embodiment, the functionality of the composition and/or system comprises single gene or genomic region functionality, such as gene or genomic region activity. In an embodiment, the functionality of the composition and/or system comprises multiple gene or genomic region functionality, such as gene or genomic region activity. In an embodiment, the functionality of the composition and/or system comprises modulation gene activity or accessibility optionally leading to transcriptional and/or epigenetic gene or genomic region activation or gene or genomic region silencing. In an embodiment, the functionality of the composition and/or system comprises modulation single gene activity or accessibility optionally leading to transcriptional and/or epigenetic gene or genomic region activation or gene or genomic region silencing. In an embodiment, the functionality of the composition and/or system comprises modulation multiple gene activity or accessibility optionally leading to transcriptional and/or epigenetic gene or genomic region activation or gene or genomic region silencing.

Optimization of selected parameters or variables in the methods as described herein may result in optimized or improved the system, such as Cas5-HNH or Cas8-HNH polypeptide nuclease-based therapy or therapeutic, specificity, efficacy, and/or safety. In an embodiment, one or more of the following parameters or variables are taken into account, are selected, or are optimized in the methods of the invention as described herein: Cas5-HNH or Cas8-HNH polypeptide nuclease allosteric interactions, Cas5-HNH or Cas8-HNH polypeptide nuclease functional domains and functional domain interactions, Cas5-HNH or Cas8-HNH polypeptide nuclease specificity, hRNA specificity, composition specificity, PAM restrictiveness, PAM type (natural or modified), PAM nucleotide content, PAM length, Cas5-HNH or Cas8-HNH polypeptide nuclease activity, hRNA activity, Cas5-HNH or Cas8-HNH polypeptide nuclease/guide complex activity, target cleavage efficiency, target site selection, target sequence length, ability of effector protein to access regions of high chromatin accessibility, degree of uniform enzyme activity across genomic targets, epigenetic tolerance, mismatch/budge tolerance, Cas5-HNH or Cas8-HNH polypeptide nuclease stability, Cas5-HNH or Cas8-HNH polypeptide nuclease mRNA stability, gRNA stability, Cas5-HNH or Cas8-HNH polypeptide nuclease complex stability, Cas5-HNH or Cas8-HNH polypeptide nuclease protein or mRNA immunogenicity or toxicity, gRNA immunogenicity or toxicity, Cas5-HNH or Cas8-HNH polypeptide nuclease immunogenicity or toxicity, Cas5-HNH or Cas8-HNH polypeptide nuclease or mRNA dose or titer, gRNA dose or titer, dose or titer, Cas5-HNH or Cas8-HNH polypeptide nuclease protein size, Cas5-HNH or Cas8-HNH polypeptide nuclease expression level, gRNA expression level, Cas5-HNH or Cas8-HNH polypeptide nuclease expression level, Cas5-HNH or Cas8-HINH polypeptide nuclease spatiotemporal expression, gRNA spatiotemporal expression, Cas5-HNH or Cas8-HNH polypeptide nuclease/hRNA spatiotemporal expression.

By means of example, and without limitation, parameter or variable optimization may be achieved as follows. Cas5-HNH or Cas8-HNH polypeptide nuclease specificity may be optimized by selecting the most specific Cas5-HNH or Cas8-HNH polypeptide nuclease, e.g., Cas5-HNH or Cas8-HNH polypeptide. This may be achieved for instance by selecting the most specific Cas5-HNH or Cas8-HNH polypeptide nuclease orthologue or by specific Cas5-HNH or Cas8-HNH polypeptide nuclease mutations which increase specificity. Guide RNA specificity may be optimized by selecting the most specific guide RNA. This can be achieved for instance by selecting guide RNA having low homology, i.e., at least one or preferably more, such as at least 2, or preferably at least 3, mismatches to off-target sites. The specificity may be optimized by increasing Cas5-HNH or Cas8-HNH polypeptide nuclease specificity and/or guide RNA specificity as above. PAM restrictiveness may be optimized by selecting a Cas5-HNH or Cas8-HNH polypeptide nuclease having to most restrictive PAM recognition. This can be achieved for instance by selecting a Cas5-HNH or Cas8-HNH polypeptide nuclease ortholog having more restrictive PAM recognition or by specific Cas5-HNH or Cas8-HNH polypeptide mutations which increase or alter PAM restrictiveness. PAM type may be optimized for instance by selecting the appropriate Cas5-HNH or Cas8-HNH polypeptide nuclease, such as the appropriate Cas5-HINH or Cas8-HNH polypeptide nuclease recognizing a desired PAM type. The Cas5-HNH or Cas8-HNH polypeptide nuclease or PAM type may be naturally occurring or may for instance be optimized based on Cas5-HNH or Cas8-HNH polypeptide nuclease mutants having an altered PAM recognition, or PAM recognition repertoire. PAM nucleotide content may for instance be optimized by selecting the appropriate Cas5-HNH or Cas8-HNH polypeptide nuclease, such as the appropriate Cas5-HNH or Cas8-HINH polypeptide nuclease recognizing a desired PAM nucleotide content. The Cas5-HNH or Cas8-HNH polypeptide nuclease or PAM type may be naturally occurring or may for instance be optimized based on Cas5-HNH or Cas8-HNH polypeptide nuclease mutants having an altered PAM recognition, or PAM recognition repertoire. PAM length may for instance be optimized by selecting the appropriate Cas5-HINH or Cas8-HINH polypeptide nuclease, such as the appropriate Cas5-HNH or Cas8-HNH polypeptide nuclease recognizing a desired PAM nucleotide length. The Cas5-HNH or Cas8-HNH polypeptide nuclease or PAM type may be naturally occurring or may for instance be optimized based on Cas5-HNH or Cas8-HNH polypeptide nuclease mutants having an altered PAM recognition, or PAM recognition repertoire.

Target length or target sequence length may be optimized, for instance, by selecting the appropriate Cas5-HNH or Cas8-HNH polypeptide nuclease, such as the appropriate Cas5-HNH or Cas8-HNH polypeptide nuclease recognizing a desired target or target sequence nucleotide length. Alternatively, or in addition, the target (sequence) length may be optimized by providing a target having a length deviating from the target (sequence) length typically associated with the Cas5-HNH or Cas8-HNH polypeptide nuclease, such as the naturally occurring Cas5-HNH or Cas8-HNH polypeptide nuclease. The Cas5-HNH or Cas8-HNH polypeptide nuclease or target (sequence) length may be naturally occurring or may for instance be optimized based on Cas5-HNH or Cas8-HNH polypeptide nuclease mutants having an altered target (sequence) length recognition, or target (sequence) length recognition repertoire. For instance, increasing or decreasing target (sequence) length may influence target recognition and/or off-target recognition. Cas5-HNH or Cas8-HNH polypeptide nuclease activity may be optimized by selecting the most active Cas5-HNH or Cas8-HNH polypeptide nuclease. This may be achieved for instance by selecting the most active Cas5-HNH or Cas8-HNH polypeptide nuclease ortholog or by specific Cas5-HNH or Cas8-HNH polypeptide nuclease mutations which increase activity. The ability of the Cas5-HNH or Cas8-HNH polypeptide nuclease protein to access regions of high chromatin accessibility, may be optimized by selecting the appropriate Cas5-HNH or Cas8-HNH polypeptide nuclease or mutant thereof, and can consider the size of the Cas5-HNH or Cas8-HNH polypeptide nuclease, charge, or other dimensional variables etc. The degree of uniform Cas5-HNH or Cas8-HINH polypeptide nuclease activity may be optimized by selecting the appropriate Cas5-HNH or Cas8-HNH polypeptide nuclease or mutant thereof, and can consider Cas5-HNH or Cas8-HNH polypeptide nuclease specificity and/or activity, PAM specificity, target length, mismatch tolerance, epigenetic tolerance, Cas5-HNH or Cas8-HNH polypeptide nuclease and/or guide RNA stability and/or half-life, Cas5-HNH or Cas8-HNH polypeptide nuclease and/or guide RNA immunogenicity and/or toxicity, etc. guide RNA activity may be optimized by selecting the most active guide RNA. In one embodiment, this can be achieved by increasing guide RNA stability through RNA modification. compositions activity may be optimized by increasing Cas5-HNH or Cas8-HNH polypeptide nuclease activity and/or guide RNA activity as above.

The target site selection may be optimized by selecting the optimal position of the target site within a gene, locus or other genomic region. The target site selection may be optimized by optimizing target location comprises selecting a target sequence with a gene, locus, or other genomic region having low variability. This may be achieved for instance by selecting a target site in an early and/or conserved exon or domain (i.e., having low variability, such as polymorphisms, within a population).

In an embodiment, optimizing target (sequence) length comprises selecting a target sequence within one or more target loci between 5 and 25 nucleotides. In an embodiment, a target sequence is 20 nucleotides.

In an embodiment, optimizing target specificity comprises selecting targets loci that minimize off-target candidates.

In one embodiment, the target site may be selected by minimization of off-target effects (e.g., off-targets qualified as having 1-5, 1-4, or preferably 1-3 mismatches compared to target and/or having one or more PAM mismatches, such as distal PAM mismatches), preferably also considering variability within a population. Cas5-HNH or Cas8-HNH polypeptide nuclease stability may be optimized by selecting Cas5-HNH or Cas8-HNH polypeptide nuclease having appropriate half-life, such as preferably a short half-life while still capable of maintaining sufficient activity. In one embodiment, this can be achieved by selecting an appropriate Cas5-HNH or Cas8-HNH polypeptide nuclease orthologue having a specific half-life or by specific Cas5-HNH or Cas8-HNH polypeptide nuclease mutations or modifications which affect half-life or stability, such as inclusion (e.g., fusion) of stabilizing or destabilizing domains or sequences. Cas5-HNH or Cas8-HNH polypeptide nuclease mRNA stability may be optimized by increasing or decreasing Cas5-HNH or Cas8-HNH polypeptide nuclease mRNA stability. In one embodiment, this can be achieved by increasing or Cas5-HNH or Cas8-HNH polypeptide nuclease mRNA stability through mRNA modification. hRNA stability may be optimized by increasing or decreasing guide RNA stability. In one embodiment, this can be achieved by increasing or decreasing guide RNA stability through RNA modification. The stability may be optimized by increasing or decreasing Cas5-HNH or Cas8-HNH polypeptide nuclease stability and/or gRNA stability as above. Cas5-HNH or Cas8-HNH polypeptide nuclease protein or mRNA immunogenicity or toxicity may be optimized by decreasing Cas5-HNH or Cas8-HNH polypeptide nuclease or mRNA immunogenicity or toxicity. In one embodiment, this can be achieved by mRNA or protein modifications. Similarly, in case of DNA based expression systems, DNA immunogenicity or toxicity may be decreased. Guide RNA immunogenicity or toxicity may be optimized by decreasing hRNA immunogenicity or toxicity. In one embodiment, this can be achieved by guide RNA modifications. Similarly, in case of DNA based expression systems, DNA immunogenicity or toxicity may be decreased. The immunogenicity or toxicity may be optimized by decreasing Cas5-HNH or Cas8-HNH polypeptide nuclease immunogenicity or toxicity and/or guide RNA immunogenicity or toxicity as above, or by selecting the least immunogenic or toxic Cas5-HNH or Cas8-HNH polypeptide nuclease/hRNA combination. Similarly, in case of DNA based expression systems, DNA immunogenicity or toxicity may be decreased. Cas5-HNH or Cas8-HINH polypeptide nuclease protein or mRNA dose or titer may be optimized by selecting dosage or titer to minimize toxicity and/or maximize specificity and/or efficacy. Guide RNA dose or titer may be optimized by selecting dosage or titer to minimize toxicity and/or maximize specificity and/or efficacy. The composition dose or titer may be optimized by selecting dosage or titer to minimize toxicity and/or maximize specificity and/or efficacy. The Cas5-HNH or Cas8-HNH polypeptide nuclease size may be optimized by selecting minimal protein size to increase efficiency of delivery, in particular for virus mediated delivery. Cas5-HNH or Cas8-HNH polypeptide nuclease, guide RNA, or complex thereof expression level may be optimized by limiting (or extending) the duration of expression and/or limiting (or increasing) expression level. This may be achieved for instance by using self-inactivating compositions, systems,, such as including a self-targeting (e.g. Cas5-HNH or Cas8-HNH polypeptide nuclease targeting) gRNA, by using viral vectors having limited expression duration, by using appropriate promoters for low (or high) expression levels, by combining different delivery methods for individual Cas5-HNH or Cas8-HNH polypeptide system components, such as virus mediated delivery of Cas5-HNH or Cas8-HNH polypeptide nuclease encoding nucleic acid combined with non-virus mediated delivery of guide RNA, or virus mediated delivery of guide RNA combined with non-virus mediated delivery of Cas5-HNH or Cas8-HNH polypeptide nuclease or mRNA. Cas5-HNH or Cas8-HNH polypeptide nuclease, guide RNA, or Cas5-HNH or Cas8-HNH polypeptide complex spatiotemporal expression may be optimized by appropriate choice of conditional and/or inducible expression systems, including controllable Cas5-HNH or Cas8-HNH polypeptide nuclease activity optionally a destabilized Cas5-HNH or Cas8-HNH polypeptide nuclease and/or a split Cas5-HNH or Cas8-HNH polypeptide nuclease, and/or cell- or tissue-specific expression systems.

In an aspect, the invention relates to a method as described herein, comprising selection of one or more (therapeutic) target, selecting the functionality of the composition and/or system, selecting composition mode of delivery, selecting composition delivery vehicle or expression system, and optimization of selected parameters or variables associated with the composition and/or its functionality, optionally wherein the parameters or variables are one or more selected from Cas5-HNH or Cas8-HNH polypeptide nuclease specificity, guide RNA specificity, Cas5-HNH or Cas8-HNH polypeptide complex specificity, PAM restrictiveness, PAM type (natural or modified), PAM nucleotide content, PAM length, Cas5-HNH or Cas8-HNH polypeptide nuclease activity, gRNA activity, Cas5-HNH or Cas8-HNH polypeptide nuclease/hRNA complex activity, target cleavage efficiency, target site selection, target sequence length, ability of effector protein to access regions of high chromatin accessibility, degree of uniform enzyme activity across genomic targets, epigenetic tolerance, mismatch/budge tolerance, Cas5-HNH or Cas8-HNH polypeptide nuclease stability, Cas5-HNH or Cas8-HNH polypeptide nuclease mRNA stability, guide RNA stability, Cas5-HNH or Cas8-HNH polypeptide complex stability, Cas5-HINH or Cas8-HNH polypeptide nuclease protein or mRNA immunogenicity or toxicity, guide RNA immunogenicity or toxicity, Cas5-HNH or Cas8-HNH polypeptide nuclease/guide RNA complex immunogenicity or toxicity, Cas5-HNH or Cas8-HNH polypeptide nuclease protein or mRNA dose or titer, guide RNA dose or titer, Cas5-HNH or Cas8-HNH polypeptide complex dose or titer, Cas5-HNH or Cas8-HNH polypeptide nuclease protein size, Cas5-HNH or Cas8-HNH polypeptide nuclease expression level, guide RNA expression level, Cas5-HNH or Cas8-HNH polypeptide nuclease/guide RNA molecule complex expression level, Cas5-HNH or Cas8-HNH polypeptide nuclease spatiotemporal expression, guide RNA spatiotemporal expression, Cas5-HNH or Cas8-HNH polypeptide nuclease/guide RNA complex spatiotemporal expression.

It will be understood that the parameters or variables to be optimized as well as the nature of optimization may depend on the (therapeutic) target, the functionality of the composition and/or system, the system mode of delivery, and/or the composition delivery vehicle or expression system.

In an aspect, the invention relates to a method as described herein, comprising optimization of guide RNA specificity at the population level. Preferably, said optimization of guide RNA specificity comprises minimizing guide RNA target site sequence variation across a population and/or minimizing guide RNA off-target incidence across a population.

In one embodiment, optimization can result in selection of a Cas5-HNH or Cas8-HNH polypeptide nuclease that is naturally occurring or is modified. In one embodiment, optimization can result in selection of a Cas5-HNH or Cas8-HNH polypeptide nuclease that has nuclease, nickase, deaminase, transposase, and/or has one or more effector functionalities deactivated or eliminated. In one embodiment, optimizing a PAM specificity can include selecting a Cas5-HNH or Cas8-HNH polypeptide nuclease with a modified PAM specificity. In one embodiment, optimizing can include selecting a Cas5-HNH or Cas8-HNH polypeptide nuclease having a minimal size. In an embodiment, optimizing effector protein stability comprises selecting an effector protein having a short half-life while maintaining sufficient activity, such as by selecting an appropriate Cas5-HNH or Cas8-HNH polypeptide nuclease orthologue having a specific half-life or stability. In an embodiment, optimizing immunogenicity or toxicity comprises minimizing effector protein immunogenicity or toxicity by protein modifications. In an embodiment, optimizing functional specific comprises selecting a protein effector with reduced tolerance of mismatches and/or bulges between the guide RNA and one or more target loci.

In an embodiment, optimizing efficacy comprises optimizing overall efficiency, epigenetic tolerance, or both. In an embodiment, maximizing overall efficiency comprises selecting an effector protein with uniform enzyme activity across target loci with varying chromatin complexity, selecting an effector protein with enzyme activity limited to areas of open chromatin accessibility. In an embodiment, chromatin accessibility is measured using one or more of ATAC-seq, or a DNA-proximity ligation assay. In an embodiment, optimizing epigenetic tolerance comprises optimizing methylation tolerance, epigenetic mark competition, or both. In an embodiment, optimizing methylation tolerance comprises selecting an effector protein that modify methylated DNA. In an embodiment, optimizing epigenetic tolerance comprises selecting an effector protein unable to modify silenced regions of a chromosome, selecting an effector protein able to modify silenced regions of a chromosome, or selecting target loci not enriched for epigenetic markers.

In an embodiment, selecting an optimized guide RNA comprises optimizing gRNA stability, gRNA immunogenicity, or both, or other gRNA associated parameters or variables as described herein elsewhere.

In an embodiment, optimizing gRNA stability and/or gRNA immunogenicity comprises RNA modification, or other gRNA associated parameters or variables as described herein elsewhere. In an embodiment, the modification comprises removing 1-3 nucleotides form the 3′ end of a target complementarity region of the gRNA. In an embodiment, modification comprises an extended gRNA and/or trans RNA/DNA element that create stable structures in the gRNA that compete with gRNA base pairing at a target of off-target loci, or extended complimentary nucleotides between the gRNA and target sequence, or both.

In an embodiment, the mode of delivery comprises delivering gRNA and/or Cas5-HNH or Cas8-HNH polypeptide nuclease, delivering gRNA and/or Cas5-HNH or Cas8-HNH polypeptide nuclease mRNA, or delivery gRNA and/or Cas5-HNH or Cas8-HNH polypeptide nuclease as a DNA based expression system. In an embodiment, the mode of delivery further comprises selecting a delivery vehicle and/or expression systems from the group consisting of liposomes, lipid particles, nanoparticles, biolistics, or viral-based expression/delivery systems. In an embodiment, expression is spatiotemporal expression is optimized by choice of conditional and/or inducible expression systems, including controllable Cas5-HNH or Cas8-HNH polypeptide nuclease activity optionally a destabilized Cas5-HNH or Cas8-HNH polypeptide and/or a split Cas5-HNH or Cas8-HNH polypeptide nuclease, and/or cell- or tissue-specific expression system.

The methods as described herein may further involve selection of the mode of delivery. In an embodiment, guide RNA and/or Cas5-HNH or Cas8-HNH polypeptide nucleases are or are to be delivered. In an embodiment, guide RNA and/or Cas5-HNH or Cas8-HNH polypeptide nuclease mRNA are or are to be delivered. In an embodiment, guide RNA and/or Cas5-HNH or Cas8-HNH polypeptide nuclease provided in a DNA-based expression system are or are to be delivered. In an embodiment, delivery of the individual system components comprises a combination of the above modes of delivery. In an embodiment, delivery comprises delivering guide RNA and/or Cas5-HNH or Cas8-HNH polypeptide nuclease protein, delivering guide RNA and/or Cas5-HNH or Cas8-HNH polypeptide nuclease mRNA, or delivering guide RNA and/or Cas5-HNH or Cas8-HNH polypeptide nuclease as a DNA based expression system.

The methods as described herein may further involve selection of the composition delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g., adenoviral, AAV, lentiviral), etc. the skilled person will understand that selection of the mode of delivery, as well as delivery vehicle or expression system may depend on for instance the cell or tissues to be targeted. In an embodiment, the delivery vehicle and/or expression system for delivering the compositions, systems, or components thereof comprises liposomes, lipid particles, nanoparticles, biolistics, or viral-based expression/delivery systems.

### Considerations for Therapeutic Applications

A consideration in genome editing therapy is the choice of sequence-specific nuclease, such as a variant of a Cas5-HNH or Cas8-HNH polypeptide nuclease. Each nuclease variant may possess its own unique set of strengths and weaknesses, many of which must be balanced in the context of treatment to maximize therapeutic benefit. For a specific editing therapy to be efficacious, a sufficiently high level of modification must be achieved in target cell populations to reverse disease symptoms. This therapeutic modification ‘threshold’ is determined by the fitness of edited cells following treatment and the amount of gene product necessary to reverse symptoms. With regard to fitness, editing creates three potential outcomes for treated cells relative to their unedited counterparts: increased, neutral, or decreased fitness. In the case of increased fitness, corrected cells may be able and expand relative to their diseased counterparts to mediate therapy. In this case, where edited cells possess a selective advantage, even low numbers of edited cells can be amplified through expansion, providing a therapeutic benefit to the patient. Where the edited cells possess no change in fitness, an increase the therapeutic modification threshold can be warranted. As such, significantly greater levels of editing may be needed to treat diseases, where editing creates a neutral fitness advantage, relative to diseases where editing creates increased fitness for target cells. If editing imposes a fitness disadvantage, as would be the case for restoring function to a tumor suppressor gene in cancer cells, modified cells would be outcompeted by their diseased counterparts, causing the benefit of treatment to be low relative to editing rates. This may be overcome with supplemental therapies to increase the potency and/or fitness of the edited cells relative to the diseased counterparts.

In addition to cell fitness, the amount of gene product necessary to treat disease can also influence the minimal level of therapeutic genome editing that can treat or prevent a disease or a symptom thereof. In cases where a small change in the gene product levels can result in significant changes in clinical outcome, the minimal level of therapeutic genome editing is less relative to cases where a larger change in the gene product levels is needed to gain a clinically relevant response. In one embodiment, the minimal level of therapeutic genome editing can range from 0.1 to 1%, 1-5%, 5-10%, 10-15%, 15-20%, 20-25%, 25-30%, 30-35%, 35-40%, 40-45%. 45-50%, or 50-55%. Thus, where a small change in gene product levels can influence clinical outcomes and diseases where there is a fitness advantage for edited cells, are ideal targets for genome editing therapy, as the therapeutic modification threshold is low enough to permit a high chance of success.

The activity of NHEJ and HDR DSB repair can vary by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells [Ciccia, A. & Elledge, S. Molecular cell 40, 179-204 (2010); Chapman, J. R., et al. Molecular cell 47, 497-510 (2012)].

The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193 (2002); Gaspar, H. B., et al. Lancet 364, 2181-2187 (2004); Beumer, K. J., et al. G3 (2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826 (2008)]. HDR also imposes a delivery challenge not seen with NHEJ strategies, as it uses the concurrent delivery of nucleases and repair templates. Thus, these differences can be kept in mind when designing, optimizing, and/or selecting a Cas5-HNH or Cas8-HNH polypeptide nuclease based therapeutic as described in greater detail elsewhere herein.

Cas5-HNH or Cas8-HNH polypeptide nuclease-based polynucleotide modification application can include combinations of proteins, small RNA molecules, and/or repair templates, and can make, In one embodiment, delivery of these multiple parts substantially more challenging than, for example, traditional small molecule therapeutics. Two main strategies for delivery of compositions, systems, and components thereof have been developed: ex vivo and in vivo. In one embodiment of ex vivo treatments, diseased cells are removed from a subject, edited and then transplanted back into the patient. In other embodiments, cells from a healthy allogeneic donor are collected, modified using a composition or component thereof, to impart various functionalities and/or reduce immunogenicity, and administered to an allogeneic recipient in need of treatment. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

In vivo polynucleotide modification via compositions, systems, and/or components thereof involves direct delivery of the compositions, systems, and/or components thereof to cell types in their native tissues. In vivo polynucleotide modification via compositions, systems, and/or components thereof allows diseases in which the affected cell population is not amenable to ex vivo manipulation to be treated. Furthermore, delivering compositions, systems, and/or components thereof to cells in situ allows for the treatment of multiple tissue and cell types.

In one embodiment, such as those where viral vector systems are used to generate viral particles to deliver the composition and/or component thereof to a cell, the total cargo size of the composition and/or component thereof should be considered as vector systems can have limits on the size of a polynucleotide that can be expressed therefrom and/or packaged into cargo inside of a viral particle. In one embodiment, the tropism of a vector system, such as a viral vector system, should be considered as it can impact the cell type to which the composition or component thereof can be efficiently and/or effectively delivered.

When delivering a system or component thereof via a viral-based system, it can be important to consider the amount of viral particles that will be needed to achieve a therapeutic effect so as to account for the potential immune response that can be elicited by the viral particles when delivered to a subject or cell(s). When delivering a system or component thereof via a viral based system, it can be important to consider mechanisms of controlling the distribution and/or dosage of the system in vivo. Generally, to reduce the potential for off-target effects, it is optimal but not necessarily required, that the amount of the system be as close to the minimum or least effective dose. In practice this can be challenging to do.

In one embodiment, it can be important to consider the immunogenicity of the system or component thereof. In embodiments, where the immunogenicity of the system or component thereof is of concern, the immunogenicity system or component thereof can be reduced. By way of example only, the immunogenicity of the system or component thereof can be reduced using the approach set out in Tangri et al. Accordingly, directed evolution or rational design may be used to reduce the immunogenicity of the Cas5-HNH or Cas8-HNH polypeptide nuclease in the host species (human or other species).

### Xenotransplantation

The present invention also contemplates use of the composition described herein, e.g., Cas5-HNH or Cas8-HNH polypeptide nuclease protein systems, to provide RNA-guided DNA nucleases adapted to be used to provide modified tissues for transplantation. For example, RNA-guided DNA nucleases may be used to knockout, knockdown or disrupt selected genes in an animal, such as a transgenic pig (such as the human heme oxygenase-1 transgenic pig line), for example by disrupting expression of genes that encode epitopes recognized by the human immune system, i.e., xenoantigen genes. Candidate porcine genes for disruption may for example include α(1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

Embodiments of the invention also relate to methods and compositions related to knocking out genes, amplifying genes and repairing particular mutations associated with DNA repeat instability and neurological disorders (Robert D. Wells, Tetsuo Ashizawa, Genetic Instabilities and Neurological Diseases, Second Edition, Academic Press, Oct. 13, 2011-Medical). Specific aspects of tandem repeat sequences have been found to be responsible for more than twenty human diseases (New insights into repeat instability: role of RNA·DNA hybrids. McIvor E I, Polak U, Napierala M. RNA Biol. 2010 September-October;7 (5): 551-8). The present effector protein systems may be harnessed to correct these defects of genomic instability.

Several further aspects of the invention relate to correcting defects associated with a wide range of genetic diseases which are further described on the website of the National Institutes of Health under the subsection Genetic Disorders (website at topic health.nih.gov/topic/GeneticDisorders). The genetic brain diseases may include but are not limited to Adrenoleukodystrophy, Agenesis of the Corpus Callosum, Aicardi Syndrome, Alpers' Disease, Alzheimer's Disease, Barth Syndrome, Batten Disease, CADASIL, Cerebellar Degeneration, Fabry's Disease, Gerstmann-Straussler-Scheinker Disease, Huntington's Disease and other Triplet Repeat Disorders, Leigh's Disease, Lesch-Nyhan Syndrome, Menkes Disease, Mitochondrial Myopathies and NINDS Colpocephaly. These diseases are further described on the website of the National Institutes of Health under the subsection Genetic Brain Disorders.

### Applications in Plants and Fungi

The compositions, systems, and methods described herein can be used to perform gene or genome interrogation or editing or manipulation in plants and fungi. For example, the applications include investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant or fugus genome. There can accordingly be improved production of plants, new plants with new combinations of traits or characteristics or new plants with enhanced traits. The compositions, systems, and methods can be used with regard to plants in Site-Directed Integration (SDI) or Gene Editing (GE) or any Near Reverse Breeding (NRB) or Reverse Breeding (RB) techniques.

The compositions, systems, and methods herein may be used to confer desired traits (e.g., enhanced nutritional quality, increased resistance to diseases and resistance to biotic and abiotic stress, and increased production of commercially valuable plant products or heterologous compounds) on essentially any plants and fungi, and their cells and tissues. The compositions, systems, and methods may be used to modify endogenous genes or to modify their expression without the permanent introduction into the genome of any foreign gene.

In one embodiment, compositions, systems, and methods may be used in genome editing in plants or where RNAi or similar genome editing techniques have been used previously; see, e.g., Nekrasov, “Plant genome editing made easy: targeted mutagenesis in model and crop plants using the CRISPR-Cas system,” Plant Methods 2013, 9:39 (doi: 10.1186/1746-4811-9-39); Brooks, “Efficient gene editing in tomato in the first generation using the CRISPR-Cas9 system,” Plant Physiology September 2014 pp 114247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi: 10.1038/cr.2013.114; published online 20 Aug. 2013; Xie, “RNA-guided genome editing in plants using a CRISPR-Cas system,” Mol Plant. 2013 November;6 (6): 1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061-Agrobacterium-Mediated Plant Transformation Method; U.S. Pat. No. 7,868,149-Plant Genome Sequences and Uses Thereof and US 2009/0100536-Transgenic Plants with Enhanced Agronomic Traits, Morrell et al “Crop genomics: advances and applications,” Nat Rev Genet. 2011 Dec. 29;13 (2): 85-96, all the contents and disclosure of each of which are herein incorporated by reference in their entirety. Aspects of utilizing the compositions, systems, and methods may be analogous to the use of the composition in plants, and mention is made of the University of Arizona website “CRISPR-PLANT” (genome.arizona.edu/crispr/) (supported by Penn State and AGI).

The compositions, systems, and methods may also be used on protoplasts. A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The compositions, systems, and methods may be used for screening genes (e.g., endogenous, mutations) of interest. In some examples, genes of interest include those encoding enzymes involved in the production of a component of added nutritional value or generally genes affecting agronomic traits of interest, across species, phyla, and plant kingdom. By selectively targeting e.g., genes encoding enzymes of metabolic pathways, the genes responsible for certain nutritional aspects of a plant can be identified. Similarly, by selectively targeting genes which may affect a desirable agronomic trait, the relevant genes can be identified. Accordingly, the present invention encompasses screening methods for genes encoding enzymes involved in the production of compounds with a particular nutritional value and/or agronomic traits.

It is also understood that reference herein to animal cells may also apply, mutatis mutandis, to plant or fungal cells unless otherwise apparent; and the enzymes herein having reduced off-target effects and systems employing such enzymes can be used in plant applications, including those mentioned herein.

In some cases, nucleic acids introduced to plants and fungi may be codon optimized for expression in the plants and fungi. Methods of codon optimization include those described in Kwon K C, et al., Codon Optimization to Enhance Expression Yields Insights into Chloroplast Translation, Plant Physiol. 2016 September;172 (1): 62-77.

The components (e.g., Cas5-HNH or Cas8-HNH polypeptide nuclease) in the compositions and systems may further comprise one or more functional domains described herein. In some examples, the functional domains may be an exonuclease. Such exonuclease may increase the efficiency of the Cas5-HNH or Cas8-HNH polypeptide nuclease' function, e.g., mutagenesis efficiency. An example of the functional domain is Trex2, as described in Weiss T et al., www.biorxiv.org/content/10.1101/2020.04.11.037572v1, doi: doi.org/10.1101/2020.04.11.037572.

### Examples of Plants

The compositions, systems, and methods herein can be used to confer desired traits on essentially any plant. A wide variety of plants and plant cell systems may be engineered for the desired physiological and agronomic characteristics. In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants.

The compositions, systems, and methods may be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g., those belonging to the orders Pinales, Ginkgoales, Cycadales, Araucariales, Cupressales and Gnetales.

The compositions, systems, and methods herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna, and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

In one embodiment, target plants and plant cells for engineering include those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini.

The term plant also encompasses Algae, which are mainly photoautotrophs unified primarily by their lack of roots, leaves and other organs that characterize higher plants. The compositions, systems, and methods can be used over a broad range of “algae” or “algae cells.” Examples of algae include eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). Examples of algae species include those of Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

### Plant Promoters

In order to ensure appropriate expression in a plant cell, the components of the components and systems herein may be placed under control of a plant promoter. A plant promoter is a promoter operable in plant cells. A plant promoter is capable of initiating transcription in plant cells, whether or not its origin is a plant cell. The use of different types of promoters is envisaged.

In some examples, the plant promoter is a constitutive plant promoter, which is a promoter that is able to express the open reading frame (ORF) that it controls in all or nearly all of the plant tissues during all or nearly all developmental stages of the plant (referred to as “constitutive expression”). One example of a constitutive promoter is the cauliflower mosaic virus 35S promoter. In some examples, the plant promoter is a regulated promoter, which directs gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In some examples, the plant promoter is a tissue-preferred promoters, which can be utilized to target enhanced expression in certain cell types within a particular plant tissue, for instance vascular cells in leaves or roots or in specific cells of the seed.

Exemplary plant promoters include those obtained from plants, plant viruses, and bacteria such as Agrobacterium or Rhizobium which comprise genes expressed in plant cells. Additional examples of promoters include those described in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

In some examples, a plant promoter may be an inducible promoter, which is inducible and allows for spatiotemporal control of gene editing or gene expression may use a form of energy. The form of energy may include sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. In a particular example, of the components of a light inducible system include a Cas5-HNH or Cas8-HNH polypeptide nuclease, a light-responsive cytochrome heterodimer (e.g., from Arabidopsis thaliana), and a transcriptional activation/repression domain.

In some examples, the promoter may be a chemical-regulated promotor (where the application of an exogenous chemical induces gene expression) or a chemical-repressible promoter (where application of the chemical represses gene expression). Examples of chemical-inducible promoters include maize ln2-2 promoter (activated by benzene sulfonamide herbicide safeners), the maize GST promoter (activated by hydrophobic electrophilic compounds used as pre-emergent herbicides), the tobacco PR-1 a promoter (activated by salicylic acid), promoters regulated by antibiotics (such as tetracycline-inducible and tetracycline-repressible promoters).

### Stable Integration in the Genome of Plants

In one embodiment, polynucleotides encoding the components of the compositions and systems may be introduced for stable integration into the genome of a plant cell. In some cases, vectors or expression systems may be used for such integration. The design of the vector or the expression system can be adjusted depending on for when, where and under what conditions the guide RNA and/or the Cas5-HNH or Cas8-HNH polypeptide nuclease gene are expressed. In some cases, the polynucleotides may be integrated into an organelle of a plant, such as a plastid, mitochondrion or a chloroplast. The elements of the expression system may be on one or more expression constructs which are either circular such as a plasmid or transformation vector, or non-circular such as linear double stranded DNA.

In one embodiment, the method of integration generally comprises the steps of selecting a suitable host cell or host tissue, introducing the construct(s) into the host cell or host tissue, and regenerating plant cells or plants therefrom. In some examples, the expression system for stable integration into the genome of a plant cell may contain one or more of the following elements: a promoter element that can be used to express the RNA and/or Cas5-HNH or Cas8-HINH polypeptide nuclease in a plant cell; a 5′ untranslated region to enhance expression; an intron element to further enhance expression in certain cells, such as monocot cells; a multiple-cloning site to provide convenient restriction sites for inserting the guide RNA and/or the Cas5-HNH or Cas8-HNH polypeptide nuclease gene sequences and other desired elements; and a 3′ untranslated region to provide for efficient termination of the expressed transcript.

### Transient Expression in Plants

In one embodiment, the components of the compositions and systems may be transiently expressed in the plant cell. In some examples, the compositions and systems may modify a target nucleic acid only when both the guide RNA and the Cas5-HNH or Cas8-HNH polypeptide nuclease are present in a cell, such that genomic modification can further be controlled. As the expression of the Cas5-HNH or Cas8-HNH polypeptide nuclease is transient, plants regenerated from such plant cells typically contain no foreign DNA. In certain examples, the Cas5-HNH or Cas8-HNH polypeptide is stably expressed and the guide sequence is transiently expressed.

DNA and/or RNA (e.g., mRNA) may be introduced to plant cells for transient expression. In such cases, the introduced nucleic acid may be provided in sufficient quantity to modify the cell but do not persist after a contemplated period of time has passed or after one or more cell divisions.

The transient expression may be achieved using suitable vectors. Exemplary vectors that may be used for transient expression include a pEAQ vector (may be tailored for Agrobacterium-mediated transient expression) and Cabbage Leaf Curl virus (CaLCuV), and vectors described in Sainsbury F. et al., Plant Biotechnol J. 2009 September;7 (7): 682-93; and Yin K et al., Scientific Reports volume 5, Article number: 14926 (2015).

Combinations of the different methods described above are also envisaged.

Translocation to and/or Expression in Specific Plant Organelles

The compositions and systems herein may comprise elements for translocation to and/or expression in a specific plant organelle.

### Chloroplast Targeting

In one embodiment, it is envisaged that the compositions and systems are used to specifically modify chloroplast genes or to ensure expression in the chloroplast. The compositions and systems (e.g., Cas5-HNH or Cas8-HNH polypeptide nuclease, guide RNAs, or their encoding polynucleotides) may be transformed, compartmentalized, and/or targeted to the chloroplast. In an example, the introduction of genetic modifications in the plastid genome can reduce biosafety issues such as gene flow through pollen.

Examples of methods of chloroplast transformation include Particle bombardment, PEG treatment, and microinjection, and the translocation of transformation cassettes from the nuclear genome to the plastid. In some examples, targeting of chloroplasts may be achieved by incorporating in chloroplast localization sequence, and/or the expression construct a sequence encoding a chloroplast transit peptide (CTP) or plastid transit peptide, operably linked to the 5′ region of the sequence encoding the components of the compositions and systems. Additional examples of transforming, targeting and localization of chloroplasts include those described in WO2010061186, Protein Transport into Chloroplasts, 2010, Annual Review of Plant Biology, Vol. 61:157-180, and US20040142476, which are incorporated by reference herein in their entireties.

### Exemplary Applications in Plants

The compositions, systems, and methods may be used to generate genetic variation(s) in a plant (e.g., crop) of interest. One or more, e.g., a library of, guide RNAs targeting one or more locations in a genome may be provided and introduced into plant cells together with the Cas5-HNH or Cas8-HNH polypeptide nuclease. For example, a collection of genome-scale point mutations and gene knock-outs can be generated. In some examples, the compositions, systems, and methods may be used to generate a plant part or plant from the cells so obtained and screening the cells for a trait of interest. The target genes may include both coding and non-coding regions. In some cases, the trait is stress tolerant and the method is a method for the generation of stress-tolerant crop varieties.

In one embodiment, the compositions, systems, and methods are used to modify endogenous genes or to modify their expression. The expression of the components may induce targeted modification of the genome, either by direct activity of the Cas5-HNH or Cas8-HNH polypeptide nuclease and optionally introduction of recombination template DNA, or by modification of genes targeted. The different strategies described herein above allow Cas5-HNH or Cas8-HNH polypeptide nuclease-mediated targeted genome editing without requiring the introduction of the components into the plant genome.

In some cases, the modification may be performed without the permanent introduction into the genome of the plant of any foreign gene, including those encoding components of the composition herein, so as to avoid the presence of foreign DNA in the genome of the plant. This can be of interest as the regulatory requirements for non-transgenic plants are less rigorous. Components which are transiently introduced into the plant cell are typically removed upon crossing.

For example, the modification may be performed by transient expression of the components of the compositions and systems. The transient expression may be performed by delivering the components of the compositions and systems with viral vectors, delivery into protoplasts, with the aid of particulate molecules such as nanoparticles or CPPs.

Generation of Plants with Desired Traits

The compositions, systems, and methods herein may be used to introduce desired traits to plants. The approaches include introduction of one or more foreign genes to confer a trait of interest, editing or modulating endogenous genes to confer a trait of interest.

### Agronomic Traits

In one embodiment, crop plants can be improved by influencing specific plant traits. Examples of the traits include improved agronomic traits such as herbicide resistance, disease resistance, abiotic stress tolerance, high yield, and superior quality, pesticide-resistance, disease resistance, insect and nematode resistance, resistance against parasitic weeds, drought tolerance, nutritional value, stress tolerance, self-pollination voidance, forage digestibility biomass, and grain yield.

In one embodiment, genes that confer resistance to pests or diseases may be introduced to plants. In cases where there are endogenous genes that confer such resistance in plants, their expression and function may be enhanced (e.g., by introducing extra copies, modifications that enhance expression and/or activity).

Examples of genes that confer resistance include plant disease resistance genes (e.g., Cf-9, Pto, RSP2, SIDMR6-1), genes conferring resistance to a pest (e.g., those described in WO96/30517), Bacillus thuringiensis proteins, lectins, Vitamin-binding proteins (e.g., avidin), enzyme inhibitors (e.g., protease or proteinase inhibitors or amylase inhibitors), insect-specific hormones or pheromones (e.g., ecdysteroid or a juvenile hormone, variant thereof, a mimetic based thereon, or an antagonist or agonist thereof) or genes involved in the production and regulation of such hormone and pheromones, insect-specific peptides or neuropeptide, insect-specific venom (e.g., produced by a snake, a wasp, etc., or analog thereof), enzymes responsible for a hyperaccumulation of a monoterpene, a sesquiterpene, a steroid, hydroxamic acid, a phenylpropanoid derivative or another nonprotein molecule with insecticidal activity, enzymes involved in the modification of biologically active molecule (e.g., a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic), molecules that stimulates signal transduction, viral-invasive proteins or a complex toxin derived therefrom, developmental-arrestive proteins produced in nature by a pathogen or a parasite, a developmental-arrestive protein produced in nature by a plant, or any combination thereof.

The compositions, systems, and methods may be used to identify, screen, introduce or remove mutations or sequences lead to genetic variability that give rise to susceptibility to certain pathogens, e.g., host specific pathogens. Such approach may generate plants that are non-host resistance, e.g., the host and pathogen are incompatible or there can be partial resistance against all races of a pathogen, typically controlled by many genes and/or also complete resistance to some races of a pathogen but not to other races.

In one embodiment, the compositions, systems, and methods may be used to modify genes involved in plant diseases. Such genes may be removed, inactivated, or otherwise regulated or modified. Examples of plant diseases include those described in [0045]-[0080] of US20140213619A1, which is incorporated by reference herein in its entirety.

In one embodiment, genes that confer resistance to herbicides may be introduced to plants. Examples of genes that confer resistance to herbicides include genes conferring resistance to herbicides that inhibit the growing point or meristem, such as an imidazolinone or a sulfonylurea, genes conferring glyphosate tolerance (e.g., resistance conferred by, e.g., mutant 5-enolpyruvylshikimate-3-phosphate synthase genes, aroA genes and glyphosate acetyl transferase (GAT) genes, respectively), or resistance to other phosphono compounds such as by glufosinate (phosphinothricin acetyl transferase (PAT) genes from Streptomyces species, including Streptomyces hygroscopicus and Streptomyces viridichromogenes), and to pyridinoxy or phenoxy proprionic acids and cyclohexones by ACCase inhibitor-encoding genes), genes conferring resistance to herbicides that inhibit photosynthesis (such as a triazine (psbA and gs+ genes) or a benzonitrile (nitrilase gene), and glutathione S-transferase), genes encoding enzymes detoxifying the herbicide or a mutant glutamine synthase enzyme that is resistant to inhibition, genes encoding a detoxifying enzyme is an enzyme encoding a phosphinothricin acetyltransferase (such as the bar or pat protein from Streptomyces species), genes encoding hydroxyphenylpyruvatedioxygenases (HPPD) inhibitors, e.g., naturally occurring HPPD resistant enzymes, and genes encoding a mutated or chimeric HPPD enzyme.

In one embodiment, genes involved in Abiotic stress tolerance may be introduced to plants. Examples of genes include those capable of reducing the expression and/or the activity of poly(ADP-ribose) polymerase (PARP) gene, transgenes capable of reducing the expression and/or the activity of the PARG encoding genes, genes coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase, enzymes involved in carbohydrate biosynthesis, enzymes involved in the production of polyfructose (e.g., the inulin and levan-type), the production of alpha-1,6 branched alpha-1,4-glucans, the production of alternan, the production of hyaluronan.

In one embodiment, genes that improve drought resistance may be introduced to plants. Examples of genes Ubiquitin Protein Ligase protein (UPL) protein (UPL3), DR02, DR03, ABC transporter, and DREB1A.

### Nutritionally Improved Plants

In one embodiment, the compositions, systems, and methods may be used to produce nutritionally improved plants. In some examples, such plants may provide functional foods, e.g., a modified food or food ingredient that may provide a health benefit beyond the traditional nutrients it contains. In certain examples, such plants may provide nutraceuticals foods, e.g., substances that may be considered a food or part of a food and provides health benefits, including the prevention and treatment of disease. The nutraceutical foods may be useful in the prevention and/or treatment of diseases in animals and humans, e.g., cancers, diabetes, cardiovascular disease, and hypertension.

An improved plant may naturally produce one or more desired compounds and the modification may enhance the level or activity or quality of the compounds. In some cases, the improved plant may not naturally produce the compound(s), while the modification enables the plant to produce such compound(s). In some cases, the compositions, systems, and methods used to modify the endogenous synthesis of these compounds indirectly, e.g., by modifying one or more transcription factors that controls the metabolism of this compound.

Examples of nutritionally improved plants include plants comprising modified protein quality, content and/or amino acid composition, essential amino acid contents, oils and fatty acids, carbohydrates, vitamins and carotenoids, functional secondary metabolites, and minerals. In some examples, the improved plants may comprise or produce compounds with health benefits. Examples of nutritionally improved plants include those described in Newell-McGloughlin, Plant Physiology, July 2008, Vol. 147, pp. 939-953.

Examples of compounds that can be produced include carotenoids (e.g., α-Carotene or β-Carotene), lutein, lycopene, Zeaxanthin, Dietary fiber (e.g., insoluble fibers, β-Glucan, soluble fibers, fatty acids (e.g., @-3 fatty acids, Conjugated linoleic acid, GLA), Flavonoids (e.g., Hydroxycinnamates, flavonols, catechins and tannins), Glucosinolates, indoles, isothiocyanates (e.g., Sulforaphane), Phenolics (e.g., stilbenes, caffeic acid and ferulic acid, epicatechin), Plant stanols/sterols, Fructans, inulins, fructo-oligosaccharides, Saponins, Soybean proteins, Phytoestrogens (e.g., isoflavones, lignans), Sulfides and thiols such as diallyl sulphide, Allyl methyl trisulfide, dithiolthiones, Tannins, such as proanthocyanidins, or any combination thereof.

The compositions, systems, and methods may also be used to modify protein/starch functionality, shelf life, taste/aesthetics, fiber quality, and allergen, antinutrient, and toxin reduction traits.

Examples of genes and nucleic acids that can be modified to introduce the traits include stearyl-ACP desaturase, DNA associated with the single allele which may be responsible for maize mutants characterized by low levels of phytic acid, Tf RAP2.2 and its interacting partner SINAT2, Tf Dof1, and DOF Tf AtDof1.1 (OBP2).

### Modification of Polyploid Plants

The compositions, systems, and methods may be used to modify polyploid plants. Polyploid plants carry duplicate copies of their genomes (e.g., as many as six, such as in wheat). In some cases, the compositions, systems, and methods may be/can be multiplexed to affect all copies of a gene, or to target dozens of genes at once. For instance, the compositions, systems, and methods may be used to simultaneously ensure a loss of function mutation in different genes responsible for suppressing defenses against a disease. The modification may be simultaneous suppression the expression of the TaMLO-A1, TaMLO-BI and TaMLO-DI nucleic acid sequence in a wheat plant cell and regenerating a wheat plant therefrom, in order to ensure that the wheat plant is resistant to powdery mildew (e.g., as described in WO2015109752).

### Regulation of Fruit-Ripening

The compositions, systems, and methods may be used to regulate ripening of fruits. Ripening is a normal phase in the maturation process of fruits and vegetables. Only a few days after it starts it may render a fruit or vegetable inedible, which can bring significant losses to both farmers and consumers.

In one embodiment, the compositions, systems, and methods are used to reduce ethylene production. In some examples, the compositions, systems, and methods may be used to suppress the expression and/or activity of ACC synthase, insert an ACC deaminase gene or a functional fragment thereof, insert a SAM hydrolase gene or functional fragment thereof, suppress ACC oxidase gene expression.

Alternatively, or additionally, the compositions, systems, and methods may be used to modify ethylene receptors (e.g., suppressing ETR1) and/or Polygalacturonase (PG). Suppression of a gene may be achieved by introducing a mutation, an antisense sequence, and/or a truncated copy of the gene to the genome.

### Increasing Storage Life of Plants

In one embodiment, the compositions, systems, and methods are used to modify genes involved in the production of compounds which affect storage life of the plant or plant part. The modification may be in a gene that prevents the accumulation of reducing sugars in potato tubers. Upon high-temperature processing, these reducing sugars react with free amino acids, resulting in brown, bitter-tasting products and elevated levels of acrylamide, which is a potential carcinogen. In one embodiment, the methods provided herein are used to reduce or inhibit expression of the vacuolar invertase gene (VInv), which encodes a protein that breaks down sucrose to glucose and fructose.

### Reducing Allergens in Plants

In one embodiment, the compositions, systems, and methods are used to generate plants with a reduced level of allergens, making them safer for consumers. To this end, the compositions, systems, and methods may be used to identify and modify (e.g., suppress) one or more genes responsible for the production of plant allergens. Examples of such genes include Lol p5, as well as those in peanuts, soybeans, lentils, peas, lupin, green beans, mung beans, such as those described in Nicolaou et al., Current Opinion in Allergy and Clinical Immunology 2011; 11 (3): 222), which is incorporated by reference herein in its entirety.

### Generation of Male Sterile Plants

The compositions, systems, and methods may be used to generate male sterile plants. Hybrid plants typically have advantageous agronomic traits compared to inbred plants. However, for self-pollinating plants, the generation of hybrids can be challenging. In different plant types (e.g., maize and rice), genes have been identified which are important for plant fertility, more particularly male fertility. Plants that are as such genetically altered can be used in hybrid breeding programs.

The compositions, systems, and methods may be used to modify genes involved male fertility, e.g., inactivating (such as by introducing mutations to) genes required for male fertility. Examples of the genes involved in male fertility include cytochrome P450-like gene (MS26) or the meganuclease gene (MS45), and those described in Wan X et al., Mol Plant. 2019 Mar. 4;12 (3): 321-342; and Kim Y J, et al., Trends Plant Sci. 2018 January;23 (1): 53-65.

### Increasing the Fertility Stage in Plants

In one embodiment, the compositions, systems, and methods may be used to prolong the fertility stage of a plant such as of a rice. For instance, a rice fertility stage gene such as Ehd3 can be targeted in order to generate a mutation in the gene and plantlets can be selected for a prolonged regeneration plant fertility stage.

### Production of Early Yield of Products

In one embodiment, the compositions, systems, and methods may be used to produce early yield of the product. For example, flowering process may be modulated, e.g., by mutating flowering repressor gene such as SP5G. Examples of such approaches include those described in Soyk S, et al., Nat Genet. 2017 January;49 (1): 162-168.

### Oil and Biofuel Production

The compositions, systems, and methods may be used to generate plants for oil and biofuel production. Biofuels include fuels made from plant and plant-derived resources. Biofuels may be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. Biofuels include bioethanol and biodiesel. Bioethanol can be produced by the sugar fermentation process of cellulose (starch), which may be derived from maize and sugar cane. Biodiesel can be produced from oil crops such as rapeseed, palm, and soybean. Biofuels can be used for transportation.

### Generation of Plants for Production of Vegetable Oils and Biofuels

The compositions, systems, and methods may be used to generate algae (e.g., diatom) and other plants (e.g., grapes) that express or overexpress high levels of oil or biofuels.

In some cases, the compositions, systems, and methods may be used to modify genes involved in the modification of the quantity of lipids and/or the quality of the lipids. Examples of such genes include those involved in the pathways of fatty acid synthesis, e.g., acetyl-CoA carboxylase, fatty acid synthase, 3-ketoacyl_acyl-carrier protein synthase III, glycerol-3-phospate deshydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid: diacylglycerol acyltransferase, phoshatidate phosphatase, fatty acid thioesterase such as palmitoyi protein thioesterase, or malic enzyme activities.

In further embodiments, it is envisaged to generate diatoms that have increased lipid accumulation. This can be achieved by targeting genes that decrease lipid catabolization. Examples of genes include those involved in the activation of triacylglycerol and free fatty acids, β-oxidation of fatty acids, such as genes of acyl-CoA synthetase, 3-ketoacyl-CoA thiolase, acyl-CoA oxidase activity and phosphoglucomutase.

In some examples, algae may be modified for production of oil and biofuels, including fatty acids (e.g., fatty esters such as acid methyl esters (FAME) and fatty acid ethyl esters (FAEE)). Examples of methods of modifying microalgae include those described in Stovicek et al. Metab. Eng. Comm., 2015; 2:1; U.S. Pat. No. 8,945,839; and International Patent Publication No. WO 2015/086795.

In some examples, one or more genes may be introduced (e.g., overexpressed) to the plants (e.g., algae) to produce oils and biofuels (e.g., fatty acids) from a carbon source (e.g., alcohol). Examples of the genes include genes encoding acyl-CoA synthases, ester synthases, thioesterases (e.g., tesA, ‘tesA, tesB, fatB, fatB2, fatB3, fatA1, or fatA), acyl-CoA synthases (e.g., fadD, JadK, BH3103, pfl-4354, EAV15023, fadD1, fadD2, RPC_4074,fadDD35, fadDD22, faa39), ester synthases (e.g., synthase/acyl-CoA:diacylglycerl acyltransferase from Simmondsia chinensis, Acinetobacter sp. ADP, Alcanivorax borkumensis, Pseudomonas aeruginosa, Fundibacter jadensis, Arabidopsis thaliana, or Alkaligenes eutrophus, or variants thereof).

Additionally, or alternatively, one or more genes in the plants (e.g., algae) may be inactivated (e.g., expression of the genes is decreased). For examples, one or more mutations may be introduced to the genes. Examples of such genes include genes encoding acyl-CoA dehydrogenases (e.g., fade), outer membrane protein receptors, and transcriptional regulator (e.g., repressor) of fatty acid biosynthesis (e.g., fabR), pyruvate formate lyases (e.g., pflB), lactate dehydrogenases (e.g., IdhA).

### Organic Acid Production

In one embodiment, plants may be modified to produce organic acids such as lactic acid. The plants may produce organic acids using sugars, pentose or hexose sugars. To this end, one or more genes may be introduced (e.g., and overexpressed) in the plants. An example of such genes includes LDH gene.

In some examples, one or more genes may be inactivated (e.g., expression of the genes is decreased). For examples, one or more mutations may be introduced to the genes. The genes may include those encoding proteins involved an endogenous metabolic pathway which produces a metabolite other than the organic acid of interest and/or wherein the endogenous metabolic pathway consumes the organic acid.

Examples of genes that can be modified or introduced include those encoding pyruvate decarboxylases (pdc), fumarate reductases, alcohol dehydrogenases (adh), acetaldehyde dehydrogenases, phosphoenolpyruvate carboxylases (ppc), D-lactate dehydrogenases (d-1dh), L-lactate dehydrogenases (1-1dh), lactate 2-monooxygenases, lactate dehydrogenase, cytochrome-dependent lactate dehydrogenases (e.g., cytochrome B2-dependent L-lactate dehydrogenases).

### Enhancing Plant Properties for Biofuel Production

In one embodiment, the compositions, systems, and methods are used to alter the properties of the cell wall of plants to facilitate access by key hydrolyzing agents for a more efficient release of sugars for fermentation. By reducing the proportion of lignin in a plant the proportion of cellulose can be increased. In one embodiment, lignin biosynthesis may be downregulated in the plant so as to increase fermentable carbohydrates.

In some examples, one or more lignin biosynthesis genes may be down regulated. Examples of such genes include 4-coumarate 3-hydroxylases (C3H), phenylalanine ammonia-lyases (PAL), cinnamate 4-hydroxylases (C4H), hydroxycinnamoyl transferases (HCT), caffeic acid O-methyltransferases (COMT), caffeoyl CoA 3-O-methyltransferases (CCoAOMT), ferulate 5-hydroxylases (F5H), cinnamyl alcohol dehydrogenases (CAD), cinnamoyl CoA-reductases (CCR), 4-coumarate-CoA ligases (4CL), monolignol-lignin-specific glycosyltransferases, and aldehyde dehydrogenases (ALDH), and those described in WO 2008064289.

In some examples, plant mass that produces lower level of acetic acid during fermentation may be reduced. To this end, genes involved in polysaccharide acetylation (e.g., Cas1L and those described in WO 2010096488) may be inactivated.

### Other Microorganisms for Oils and Biofuel Production

In one embodiment, microorganisms other than plants may be used for production of oils and biofuels using the compositions, systems, and methods herein. Examples of the microorganisms include those of the genus of Escherichia, Bacillus, Lactobacillus, Rhodococcus, Synechococcus, Synechoystis, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

### Plant Cultures and Regeneration

In one embodiment, the modified plants or plant cells may be cultured to regenerate a whole plant which possesses the transformed or modified genotype and thus the desired phenotype. Examples of regeneration techniques include those relying on manipulation of certain phytohormones in a tissue culture growth medium, relying on a biocide and/or herbicide marker which has been introduced together with the desired nucleotide sequences, obtaining from cultured protoplasts, plant callus, explants, organs, pollens, embryos or parts thereof.

### Detecting Modifications in the Plant Genome-Selectable Markers

When the compositions, systems, and methods are used to modify a plant, suitable methods may be used to confirm and detect the modification made in the plant. In some examples, when a variety of modifications are made, one or more desired modifications or traits resulting from the modifications may be selected and detected. The detection and confirmation may be performed by biochemical and molecular biology techniques such as Southern analysis, PCR, Northern blot, S1 RNase protection, primer-extension or reverse transcriptase-PCR, enzymatic assays, ribozyme activity, gel electrophoresis, Western blot, immunoprecipitation, enzyme-linked immunoassays, in situ hybridization, enzyme staining, and immunostaining.

In some cases, one or more markers, such as selectable and detectable markers, may be introduced to the plants. Such markers may be used for selecting, monitoring, isolating cells and plants with desired modifications and traits. A selectable marker can confer positive or negative selection and is conditional or non-conditional on the presence of external substrates. Examples of such markers include genes and proteins that confer resistance to antibiotics, such as hygromycin (hpt) and kanamycin (nptII), and genes that confer resistance to herbicides, such as phosphinothricin (bar) and chlorosulfuron (als), enzyme capable of producing or processing a colored substances (e.g., the β-glucuronidase, luciferase, B or C1 genes).

### Applications in Fungi

The compositions, systems, and methods described herein can be used to perform efficient and cost-effective gene or genome interrogation or editing or manipulation in fungi or fungal cells, such as yeast. The approaches and applications in plants may be applied to fungi as well.

A fungal cell may be any type of eukaryotic cell within the kingdom of fungi, such as phyla of Ascomycota, Basidiomycota, Blastocladiomycota, Chytridiomycota, Glomeromycota, Microsporidia, and Neocallimastigomycota. Examples of fungi or fungal cells in include yeasts, molds, and filamentous fungi.

In one embodiment, the fungal cell is a yeast cell. A yeast cell refers to any fungal cell within the phyla Ascomycota and Basidiomycota. Examples of yeasts include budding yeast, fission yeast, and mold, S. cerervisiae, Kluyveromyces marxianus, Issatchenkia orientalis, Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, Pichia kudriavzevii and Candida acidothermophilum).

In one embodiment, the fungal cell is a filamentous fungal cell, which grow in filaments, e.g., hyphae or mycelia. Examples of filamentous fungal cells include Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In one embodiment, the fungal cell is of an industrial strain. Industrial strains include any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes include fermentation (e.g., in production of food or beverage products), distillation, biofuel production, production of a compound, and production of a polypeptide. Examples of industrial strains include, without limitation, JAY270 and ATCC4124.

In one embodiment, the fungal cell is a polyploid cell whose genome is present in more than one copy. Polyploid cells include cells naturally found in a polyploid state, and cells that has been induced to exist in a polyploid state (e.g., through specific regulation, alteration, inactivation, activation, or modification of meiosis, cytokinesis, or DNA replication). A polyploid cell may be a cell whose entire genome is polyploid, or a cell that is polyploid in a particular genomic locus of interest. In some examples, the abundance of guide RNA may more often be a rate-limiting component in genome engineering of polyploid cells than in haploid cells, and thus the methods using the composition described herein may take advantage of using certain fungal cell types.

In one embodiment, the fungal cell is a diploid cell, whose genome is present in two copies. Diploid cells include cells naturally found in a diploid state, and cells that have been induced to exist in a diploid state (e.g., through specific regulation, alteration, inactivation, activation, or modification of meiosis, cytokinesis, or DNA replication). A diploid cell may refer to a cell whose entire genome is diploid, or it may refer to a cell that is diploid in a particular genomic locus of interest.

In one embodiment, the fungal cell is a haploid cell, whose genome is present in one copy. Haploid cells include cells naturally found in a haploid state, or cells that have been induced to exist in a haploid state (e.g., through specific regulation, alteration, inactivation, activation, or modification of meiosis, cytokinesis, or DNA replication). A haploid cell may refer to a cell whose entire genome is haploid, or it may refer to a cell that is haploid in a particular genomic locus of interest.

The compositions and systems, and nucleic acid encoding thereof may be introduced to fungi cells using the delivery systems and methods herein. Examples of delivery systems include lithium acetate treatment, bombardment, electroporation, and those described in Kawai et al., 2010, Bioeng Bugs. 2010 November-December; 1 (6): 395-403.

In some examples, a yeast expression vector (e.g., those with one or more regulatory elements) may be used. Examples of such vectors include a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2u plasmids, yeast integrative plasmids, yeast replicative plasmids, shuttle vectors, and episomal plasmids.

### Biofuel and Materials Production by Fungi

In one embodiment, the compositions, systems, and methods may be used for generating modified fungi for biofuel and material productions. For instance, the modified fungi for production of biofuel or biopolymers from fermentable sugars and optionally to be able to degrade plant-derived lignocellulose derived from agricultural waste as a source of fermentable sugars. Foreign genes required for biofuel production and synthesis may be introduced into fungi. In some examples, the genes may encode enzymes involved in the conversion of pyruvate to ethanol or another product of interest, degrade cellulose (e.g., cellulase), endogenous metabolic pathways which compete with the biofuel production pathway.

In some examples, the compositions, systems, and methods may be used for generating and/or selecting yeast strains with improved xylose or cellobiose utilization, isoprenoid biosynthesis, and/or lactic acid production. One or more genes involved in the metabolism and synthesis of these compounds may be modified and/or introduced to yeast cells. Examples of the methods and genes include lactate dehydrogenase, PDC1 and PDC5, and those described in Ha, S., et al. (2011) Proc. Natl. Acad. Sci. USA 108 (2): 504-9 and Galazka, J. M., et al. (2010) Science 330 (6000): 84-6; Jakočiūnas T et al., Metab Eng. 2015 March;28:213-222; Stovicek V, et al., FEMS Yeast Res. 2017 Aug. 1;17 (5).

### Improved Plants and Yeast Cells

The present disclosure further provides improved plants and fungi. The improved and fungi may comprise one or more genes introduced, and/or one or more genes modified by the compositions, systems, and methods herein. The improved plants and fungi may have increased food or feed production (e.g., higher protein, carbohydrate, nutrient or vitamin levels), oil and biofuel production (e.g., methanol, ethanol), tolerance to pests, herbicides, drought, low or high temperatures, excessive water, etc.

The plants or fungi may have one or more parts that are improved, e.g., leaves, stems, roots, tubers, seeds, endosperm, ovule, and pollen. The parts may be viable, nonviable, regeneratable, and/or non-regeneratable.

The improved plants and fungi may include gametes, seeds, embryos, either zygotic or somatic, progeny and/or hybrids of improved plants and fungi. The progeny may be a clone of the produced plant or fungi, or may result from sexual reproduction by crossing with other individuals of the same species to introgress further desirable traits into their offspring. The cell may be in vivo or ex vivo in the cases of multicellular organisms, particularly plants.

### Further Applications in Plants

Further applications of the compositions, systems, and methods on plants and fungi include visualization of genetic element dynamics (e.g., as described in Chen B, et al., Cell. 2013 Dec. 19;155 (7): 1479-91), targeted gene disruption positive-selection in vitro and in vivo (as described in Malina A et al., Genes Dev. 2013 Dec. 1;27 (23): 2602-14), epigenetic modification such as using fusion of Cas5-HNH or Cas8-HNH polypeptide nuclease and histone-modifying enzymes (e.g., as described in Rusk N, Nat Methods. 2014 January; 11 (1): 28), identifying transcription regulators (e.g., as described in Waldrip Z J, Epigenetics. 2014 September;9 (9): 1207-11), anti-virus treatment for both RNA and DNA viruses (e.g., as described in Price A A, et al., Proc Natl Acad Sci USA. 2015 May 12;112 (19): 6164-9; Ramanan V et al., Sci Rep. 2015 Jun. 2; 5:10833), alteration of genome complexity such as chromosome numbers (e.g., as described in Karimi-Ashtiyani R et al., Proc Natl Acad Sci USA. 2015 Sep. 8;112 (36): 11211-6; Anton T, et al., Nucleus. 2014 March-April;5 (2): 163-72), self-cleavage of the composition for controlled inactivation/activation (e.g., as described Sugano S S et al., Plant Cell Physiol. 2014 March;55 (3): 475-81), multiplexed gene editing (as described in Kabadi A M et al., Nucleic Acids Res. 2014 Oct. 29;42 (19):e147), development of kits for multiplex genome editing (as described in Xing H L et al., BMC Plant Biol. 2014 Nov. 29; 14:327), starch production (as described in Hebelstrup K H et al., Front Plant Sci. 2015 Apr. 23; 6:247), targeting multiple genes in a family or pathway (e.g., as described in Ma X et al., Mol Plant. 2015 August;8 (8): 1274-84), regulation of non-coding genes and sequences (e.g., as described in Lowder L G, et al., Plant Physiol. 2015 October; 169 (2): 971-85), editing genes in trees (e.g., as described in Belhaj K et al., Plant Methods. 2013 Oct. 11;9 (1): 39; Harrison M M, et al., Genes Dev. 2014 Sep. 1;28 (17): 1859-72; Zhou X et al., New Phytol. 2015 October;208 (2): 298-301), introduction of mutations for resistance to host-specific pathogens and pests.

Additional examples of modifications of plants and fungi that may be performed using the compositions, systems, and methods include analogous modifications described in International Patent Publication Nos. WO2016/099887, WO2016/025131, WO2016/073433, WO2017/066175, WO2017/100158, WO 2017/105991, WO2017/106414, WO2016/100272, WO2016/100571, WO 2016/100568, WO 2016/100562, and WO 2017/019867.

### Applications in Non-Human Animals

The compositions, systems, and methods may be used to study and modify non-human animals, e.g., introducing desirable traits and disease resilience, treating diseases, facilitating breeding, etc. In one embodiment, the compositions, systems, and methods may be used to improve breeding and introducing desired traits, e.g., increasing the frequency of trait-associated alleles, introgression of alleles from other breeds/species without linkage drag, and creation of de novo favorable alleles. Genes and other genetic elements that can be targeted may be screened and identified. Examples of application and approaches include those described in Tait-Burkard C, et al., Livestock 2.0-genome editing for fitter, healthier, and more productive farmed animals. Genome Biol. 2018 Nov. 26;19 (1): 204; Lillico S, Agricultural applications of genome editing in farmed animals. Transgenic Res. 2019 August;28 (Suppl 2): 57-60; Houston R D, et al., Harnessing genomics to fast-track genetic improvement in aquaculture. Nat Rev Genet. 2020 Apr. 16. doi: 10.1038/s41576-020-0227-y, which are incorporated herein by reference in their entireties. Applications described in other sections such as therapeutic, diagnostic, etc. can also be used on the animals herein.

The compositions, systems, and methods may be used on animals such as fish, amphibians, reptiles, mammals, and birds. The animals may be farm and agriculture animals, or pets. Examples of farm and agriculture animals include horses, goats, sheep, swine, cattle, llamas, alpacas, and birds, e.g., chickens, turkeys, ducks, and geese. The animals may be a non-human primate, e.g., baboons, capuchin monkeys, chimpanzees, lemurs, macaques, marmosets, tamarins, spider monkeys, squirrel monkeys, and vervet monkeys. Examples of pets include dogs, cats, horses, wolfs, rabbits, ferrets, gerbils, hamsters, chinchillas, fancy rats, guinea pigs, canaries, parakeets, and parrots.

In one embodiment, one or more genes may be introduced (e.g., overexpressed) in the animals to obtain or enhance one or more desired traits. Growth hormones, insulin-like growth factors (IGF-1) may be introduced to increase the growth of the animals, e.g., pigs or salmon (such as described in Pursel V G et al., J Reprod Fertil Suppl. 1990; 40:235-45; Waltz E, Nature. 2017; 548:148). Fat-1 gene (e.g., from C elegans) may be introduced for production of larger ratio of n-3 to n-6 fatty acids may be induced, e.g., in pigs (such as described in Li M, et al., Genetics. 2018; 8:1747-54). Phytase (e.g., from E coli) xylanase (e.g., from Aspergillus niger), beta-glucanase (e.g., from bacillus lichenformis) may be introduced to reduce the environmental impact through phosphorous and nitrogen release reduction, e.g., in pigs (such as described in Golovan S P, et al., Nat Biotechnol. 2001; 19:741-5; Zhang X et al., elife. 2018). shRNA decoy may be introduced to induce avian influenza resilience e.g., in chicken (such as described in Lyall et al., Science. 2011; 331:223-6). Lysozyme or lysostaphin may be introduced to induce mastitis resilience e.g., in goat and cow (such as described in Maga E A et al., Foodborne Pathog Dis. 2006; 3:384-92; Wall R J, et al., Nat Biotechnol. 2005; 23:445-51). Histone deacetylase such as HDAC6 may be introduced to induce PRRSV resilience, e.g., in pig (such as described in Lu T., et al., PLOS One. 2017;12:e0169317). CD163 may be modified (e.g., inactivated or removed) to introduce PRRSV resilience in pigs (such as described in Prather R S et al . . . , Sci Rep. 2017 Oct. 17;7 (1): 13371). Similar approaches may be used to inhibit or remove viruses and bacteria (e.g., Swine Influenza Virus (SIV) strains which include influenza C and the subtypes of influenza A known as HIN1, H1N2, H2N1, H3N1, H3N2, and H2N3, as well as pneumonia, meningitis and oedema) that may be transmitted from animals to humans.

In one embodiment, one or more genes may be modified or edited for disease resistance and production traits. Myostatin (e.g., GDF8) may be modified to increase muscle growth, e.g., in cow, sheep, goat, catfish, and pig (such as described in Crispo M et al., PLOS One. 2015;10:e0136690; Wang X, et al., Anim Genet. 2018; 49:43-51; Khalil K, et al., Sci Rep. 2017; 7:7301; Kang J-D, et al., RSC Adv. 2017; 7:12541-9). Pc POLLED may be modified to induce horlessness, e.g., in cow (such as described in Carlson D F et al., Nat Biotechnol. 2016; 34:479-81). KISSIR may be modified to induce boar taint (hormone release during sexual maturity leading to undesired meat taste), e.g., in pigs. Dead end protein (dnd) may be modified to induce sterility, e.g., in salmon (such as described in Wargelius A, et al., Sci Rep. 2016; 6:21284). Nano2 and DDX may be modified to induce sterility (e.g., in surrogate hosts), e.g., in pigs and chicken (such as described Park K-E, et al., Sci Rep. 2017; 7:40176; Taylor L et al., Development. 2017; 144:928-34). CD163 may be modified to induce PRRSV resistance, e.g., in pigs (such as described in Whitworth K M, et al., Nat Biotechnol. 2015; 34:20-2). RELA may be modified to induce ASFV resilience, e.g., in pigs (such as described in Lillico S G, et al., Sci Rep. 2016; 6:21645). CD18 may be modified to induce Mannheimia (Pasteurella) haemolytica resilience, e.g., in cows (such as described in Shanthalingam S, et al., roc Natl Acad Sci USA. 2016; 113:13186-90). NRAMP1 may be modified to induce tuberculosis resilience, e.g., in cows (such as described in Gao Y et al., Genome Biol. 2017; 18:13). Endogenous retrovirus genes may be modified or removed for xenotransplantation such as described in Yang L, et al. Science. 2015; 350:1101-4; Niu D et al., Science. 2017; 357:1303-7). Negative regulators of muscle mass (e.g., Myostatin) may be modified (e.g., inactivated) to increase muscle mass, e.g., in dogs (as described in Zou Q et al., J Mol Cell Biol. 2015 December;7 (6): 580-3).

Animals such as pigs with severe combined immunodeficiency (SCID) may generated (e.g., by modifying RAG2) to provide useful models for regenerative medicine, xenotransplantation (discussed also elsewhere herein), and tumor development. Examples of methods and approaches include those described Lee K, et al., Proc Natl Acad Sci USA. 2014 May 20;111 (20): 7260-5; and Schomberg et al. FASEB Journal, April 2016; 30 (1): Suppl 571.1.

SNPs in the animals may be modified. Examples of methods and approaches include those described Tan W. et al., Proc Natl Acad Sci USA. 2013 Oct. 8;110 (41): 16526-31; Mali P, et al., Science. 2013 Feb. 15;339 (6121): 823-6.

Stem cells (e.g., induced pluripotent stem cells) may be modified and differentiated into desired progeny cells, e.g., as described in Heo Y T et al., Stem Cells Dev. 2015 Feb. 1;24 (3): 393-402.

Profile analysis (such as Igenity) may be performed on animals to screen and identify genetic variations related to economic traits. The genetic variations may be modified to introduce or improve the traits, such as carcass composition, carcass quality, maternal and reproductive traits and average daily gain.

Further embodiments are illustrated in the following Examples which are given for illustrative purposes only and are not intended to limit the scope of the invention.

### EXAMPLES

### Example 1

Computational efforts led by Applicants to identify CRISPR systems with unique features that distinguish them from current CRISPR types. One of the identified systems is “Cas5-HNH”, as named by Applicants. FIG. 1 exemplifies the CRISPR systems used as inputs to identify the novel Cas5-HINH output.

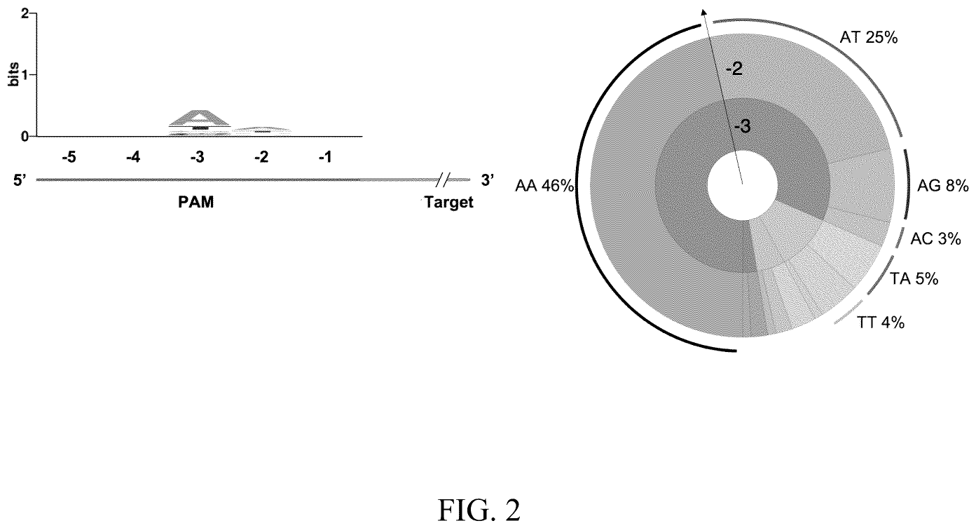

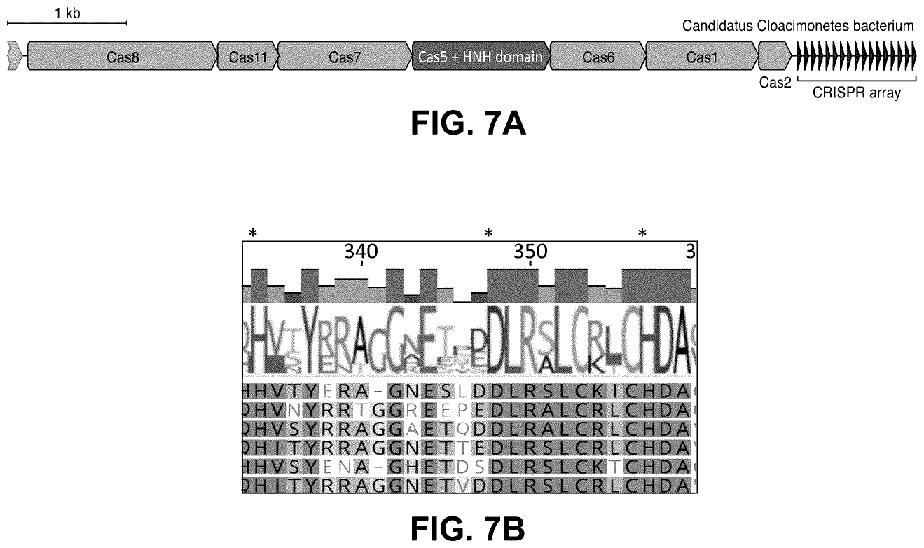

E. coli PAM interference assay: 30 uL Endura electrocompetent cells from Lucigen were electroporated with 100 ng each of a PAM library target plasmid and an expression plasmid containing the proteins shown on FIG. 7 (expressed from Lac promoter in natural operon structure)-Cas8, Cas11, Cas7, Cas5 and Cas6 are included, Cas1 and Cas2 NOT included- and an associated guide RNA (DR-spacer targeting library-DR) expressed from a J23119 promoter, with three replicate transformations. The next day, the resulting colonies were scraped from plates and plasmids were miniprepped. The resulting plasmids containing the PAM library were amplified and sequenced on an Illumina NextSeq, and depletion of specific PAMs was calculated relative to an empty control. See FIG. 2.

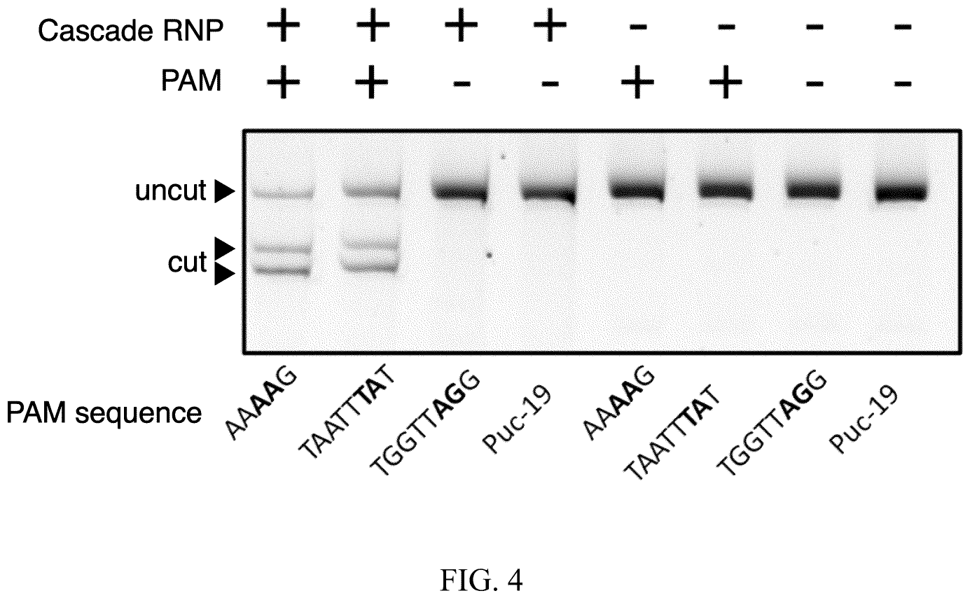

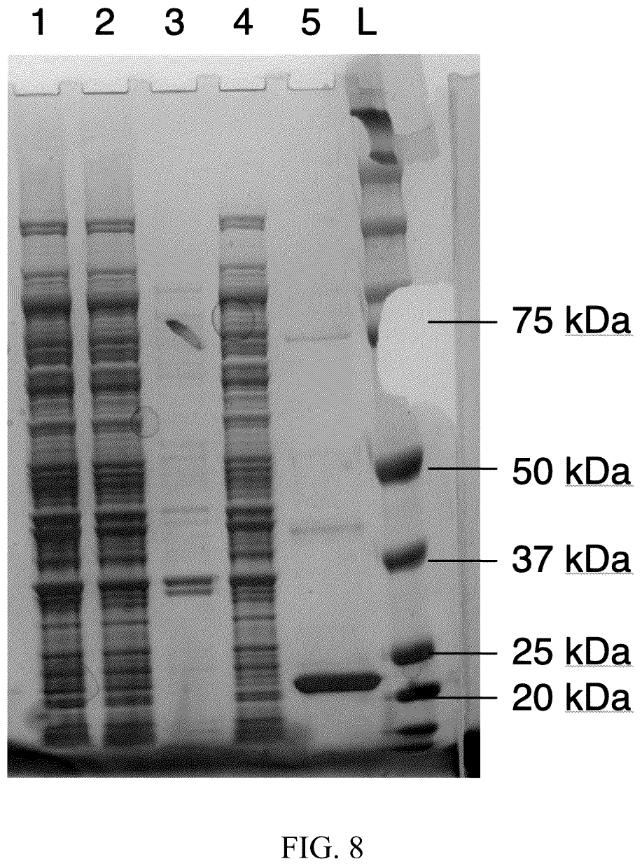

Cleavage assay: Cascade RNP complex shown in FIG. 8 was concentrated and used in an in vitro dsDNA cleavage assay. The Cascade RNP was incubated at 37 C for 1 hour with a dsDNA target complementary (shown by specific PAM sequences on bottom row labels) or noncomplementary (Puc-19 on bottom row labels) target with or without an active PAM, as determined by PAM interference assay shown on FIG. 2, in a buffer containing 20 mM Tris-HCl pH 8, 5 mM MgCl2, 1 mM ATP. Results were analyzed by gel electrophoresis on a 4% agarose E-gel, with SYBR Gold staining. See FIG. 4.

Applicants also performed protein purification. His14-tag was added to Cas11 ORF, and the full operon including Cas8, Cas11, Cas7, Cas5, Cas6 (NO Cas1 or Cas2) was expressed from a T7 promoter in BL21 (DE3) cells, along with the crRNA (DR-spacer-DR). Cascade complex was purified by pulling down on the His14 tag using Ni-NTA resin and analyzed by gel electrophoresis and Coomassie blue staining. See FIG. 8.

Mammalian genome editing assay: HEK293FT cells were transfected with 50 ng each of separate plasmids containing expression cassettes for Cas8, Cas11, Cas5, Cas7 and Cas6, each N-terminally tagged with a FLAG tag and SV40 NLS, and 50 ng of a plasmid containing crRNA expression cassette. After 3 days, gDNA was harvested, and targeted regions were amplified and sequenced on an Illumina MiSeq. The resulting sequences were analyzed for insertions and deletions (indels) compared to a reference sequence. See FIG. 5.

Mammalian genome editing assay: Genome editing assay was carried out as described on FIG. 5, except for each (−) condition, a plasmid with no expression cassette was transfected in place of the plasmid expression each of the noted genes (ex. “-cas5” means that the Cas5 expression plasmid was replaced by a filler plasmid in the transfection. For the “mut HNH” condition, catalytic residues of the HNH domain, H310, D324 and H333, were mutated to alanine in the Cas5 gene. See FIG. 6.

Other Cascade complexes only have one Cas5 subunit. However, HNH domains typically only cleave one strand of DNA (ex. HNH in Cas9 only cleaves target strand, restriction endonucleases with HNH domain dimerize for dsDNA cleavage, etc). Mapping of the cleavage site shows that cleavage occurs on the PAM-distal end of the target. However, in other Cascade structures, Cas5 is positioned on the PAM-proximal end of the complex. Cas11 subunit is apparently not necessary for activity in human cells.

### Example 2—Type I Cascade Components are Functionalized with HNH Domains for Precise dsDNA Cleavage

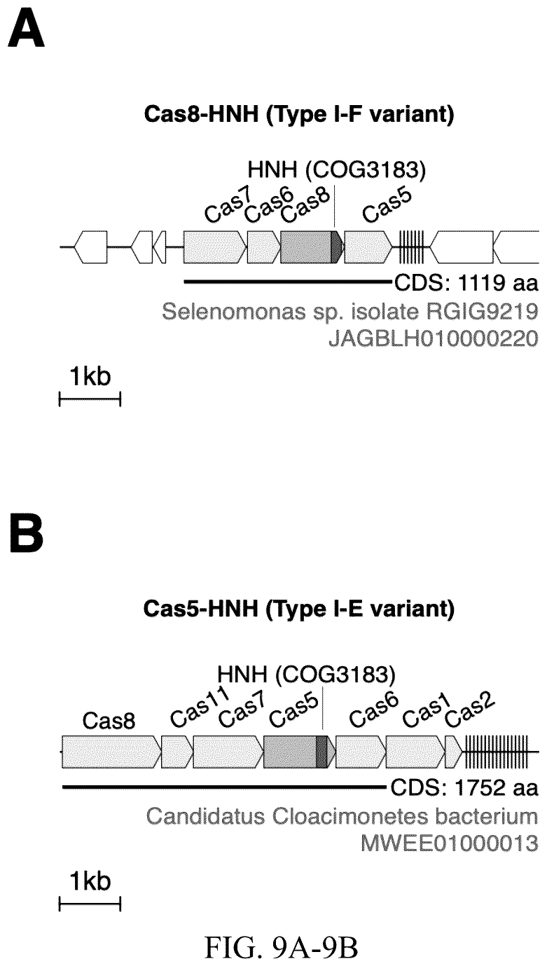

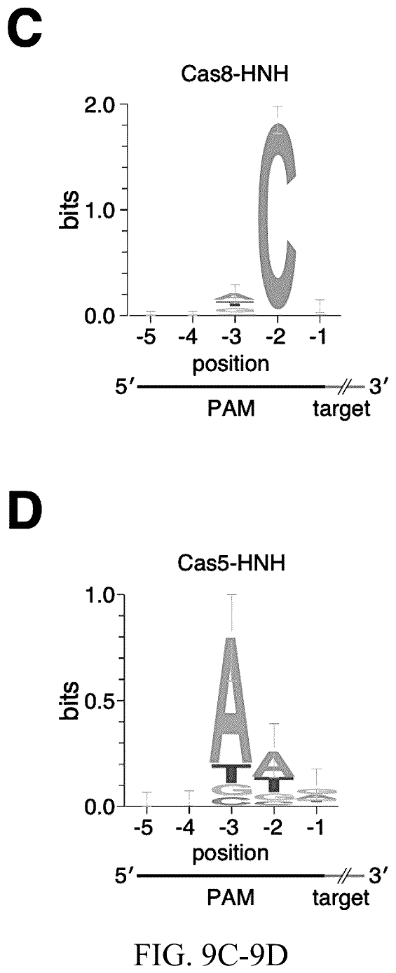

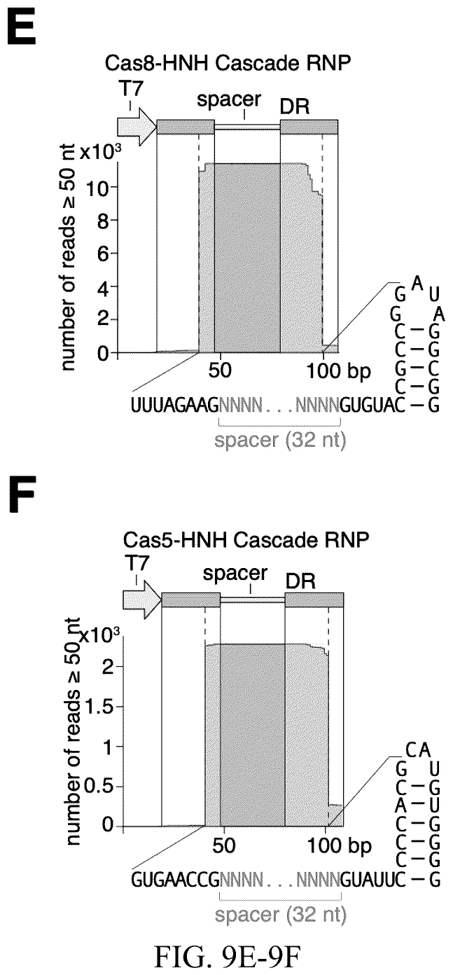

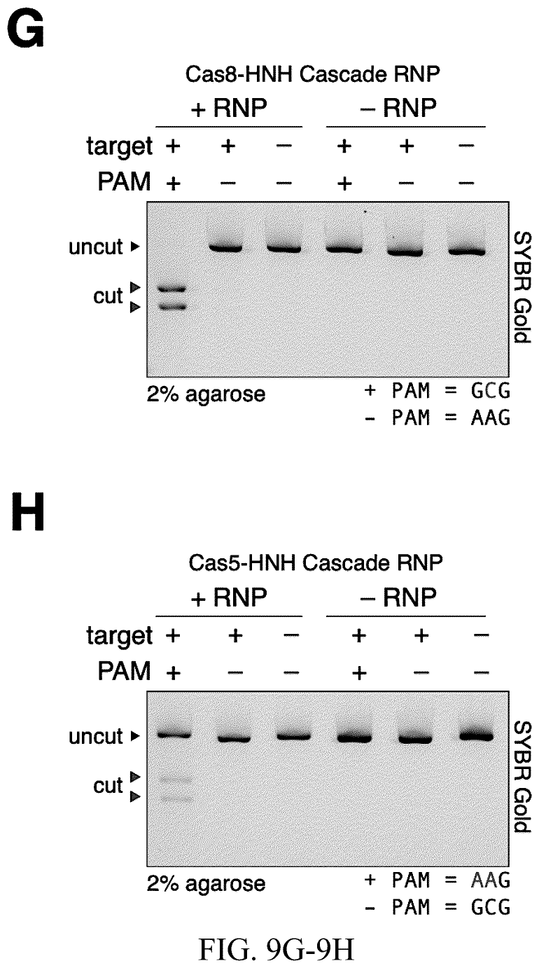

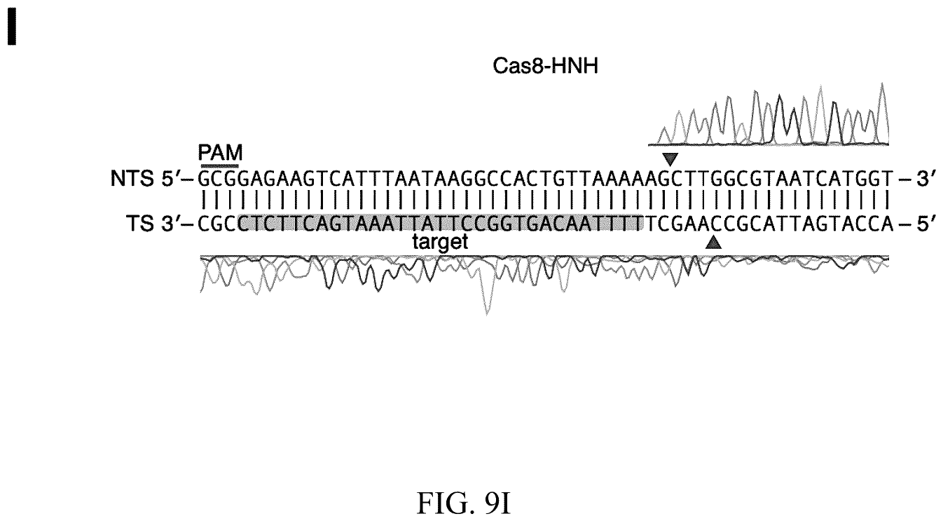

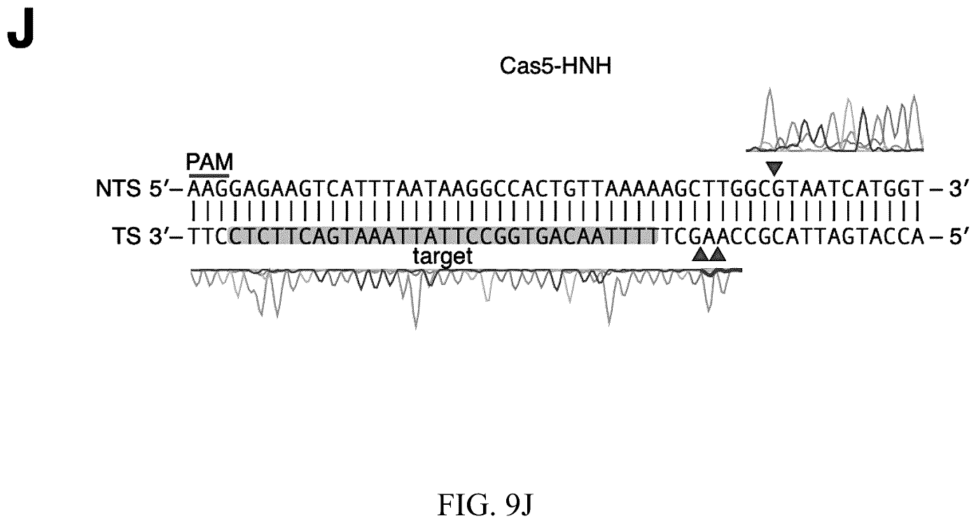

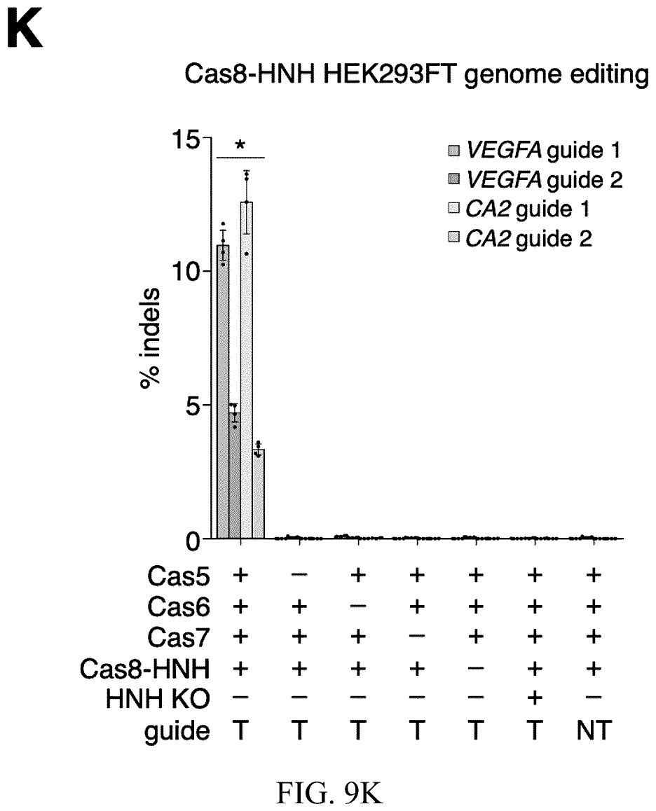

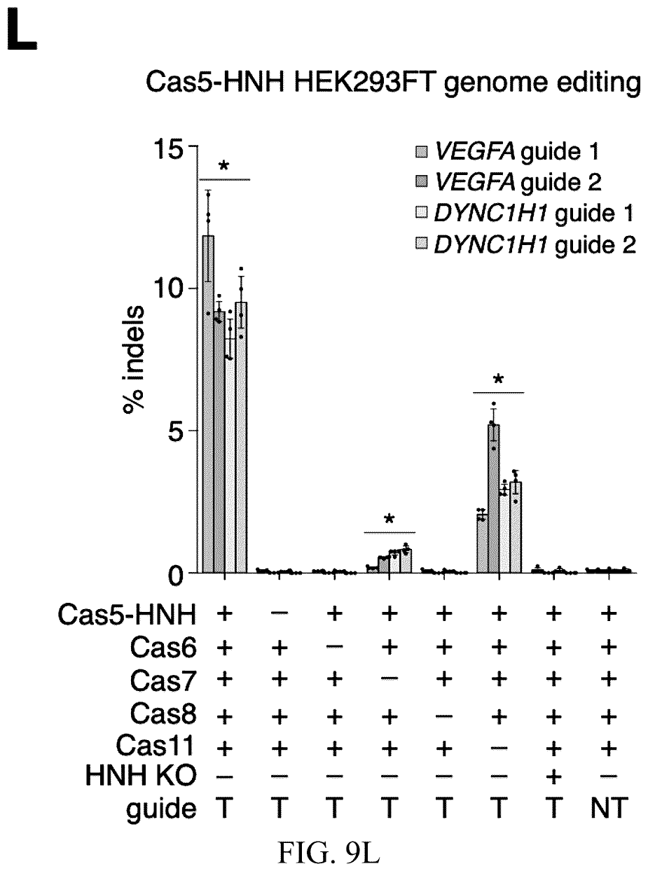

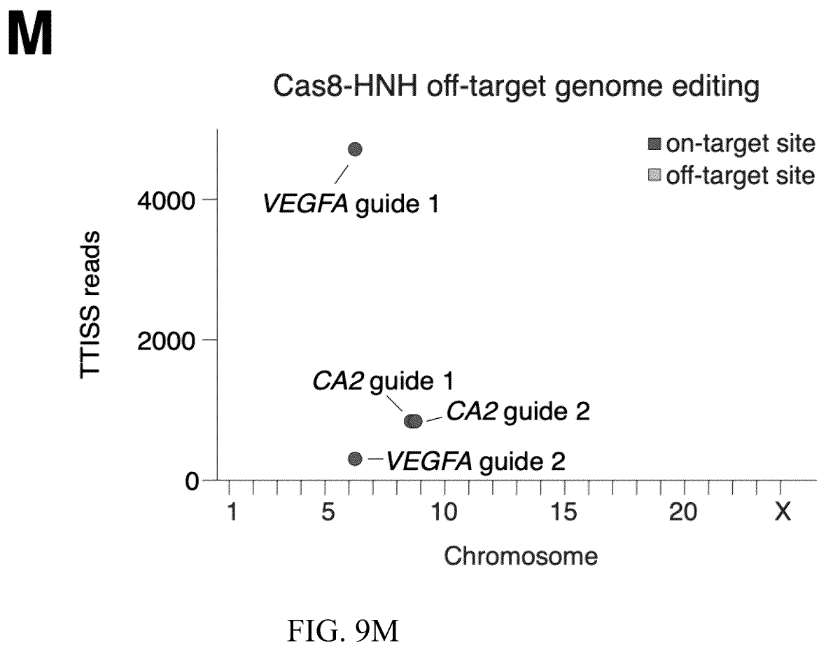

Applicants also identified two novel variants of Type I CRISPR systems lacking cas3, but containing an HNH nuclease domain inserted into one of the Cascade backbone components, either cas8 or cas5 (FIG. 9A, B). The Cas8-HNH system consists of four genes and is most closely related to Type I-F1 CRISPR systems, whereas the Cas5-HNH system consists of five genes and is most closely related to Type I-E CRISPR systems. Based on the absence of the cas3 helicase/nuclease gene along with the previously unknown association of an HNH domain, Applicants hypothesized that both of these systems might enable precise RNA-guided double-stranded DNA (dsDNA) cleavage, in contrast to the processive degradation activity exhibited by Cas3 in canonical Type I systems (1, 2).

To test this hypothesis, Applicants performed a PAM discovery assay in E. coli and observed depletion of specific PAMs for Cas8-HNH and Cas5-HNH (FIG. 9C, D), suggesting that both systems are capable of RNA-guided interference activity. Small RNA sequencing of the recombinantly purified Cascade RNPs showed that Cascade binds to crRNAs, both containing 32-nt spacers. Each crRNA includes 5′ and 3′ DR handles, consistent with other Type I CRISPR systems (FIG. 9E, F) (3).

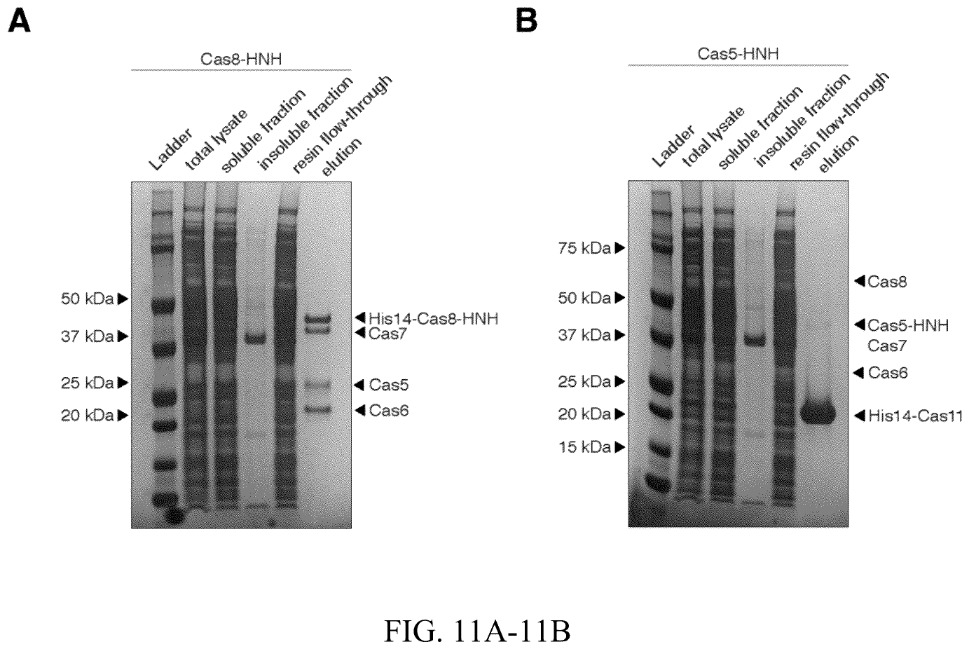

Next, Applicants confirmed the ability of the Cas8-HNH and Cas5-HNH Cascade RNPs to cleave dsDNA in a precise, PAM-dependent manner (FIG. 9G, H, FIG. 11). Sequencing of the cleavage products for each system showed that Cas8-HNH cleaves the TS and NTS 5 bp and 2 bp downstream of the protospacer, respectively, on the PAM-distal end of the target, generating 5′ overhangs (FIG. 9I). By contrast, Cas5-HNH cleaves the TS and NTS 3-4 bp and 8 bp downstream of the protospacer, respectively, on the PAM-distal end, generating 3′ overhangs (FIG. 9J). Finally, Applicants tested if Cas8-HNH and Cas5-HNH can programmably generate short insertions/deletions (indels) in mammalian cells. Applicants found that both the Cas8-HNH and Cas5-HNH systems are capable of inducing indels with varying efficiencies up to ˜13% (FIG. 9K, L, Table 2). For Cas8-HNH, all protein subunits were required for indel formation (FIG. 9K). For the Cas5-HNH system, the Cas11/Cse2 subunit was dispensable for indel formation but its deletion resulted in a reduced activity up to ˜6%, while deleting Cas7 resulted in minimal activity of up to ˜1%. Deleting any of the other components ablated activity (FIG. 9L). Inactivation of the catalytic residues of the HNH domain in each system also abolished activity, demonstrating that the HNH domain mediates target cleavage in both systems (FIG. 9K, L). To assess the genome-wide specificity of cleavage, Applicants performed tagmentation-based tag integration site sequencing (4). For Cas8-HNH, Applicants detected no off targets for the 4 tested guides, suggesting that this system is highly specific (FIG. 9M). The 3′ overhangs generated by Cas5-HNH cleavage were apparently not compatible with blunt-end ligation required for this assay.

Applicants observed many new domains and proteins associated with CRISPR effector modules, several of which appear to replace the function of lost components (FIG. 10A), highlighting the modular evolution of CRISPR effectors. Applicants identified HNH nuclease domains as additions to pre-existing CRISPR systems on two independent occasions: Cas5-HNH and Cas8-HNH (FIG. 15). The evolution of these systems mimics the origin of type II CRISPR systems, in which an HNH nuclease was inserted into the RuvC-like nuclease domain of the IsrB protein to become IscB, the likely direct ancestor of Cas9 (FIG. 10E) (5).

Due to the ability of CRISPR-Cas systems to programmably sense specific nucleic acids and subsequently enact enzymatic functions, the discovery and characterization of novel CRISPR effectors and downstream auxiliary functions has the potential to enable a wide range of applications and improve existing CRISPR-based technologies, such as genome editing. Here, Applicants characterized the genome editing activities of Cas8-HNH and Cas5-HNH nucleases, which showed striking precision and hold promise for further development as genome editing tools.

CRISPR-Cas systems have immense potential to enhance genome editing and biotechnology more broadly. Towards this goal, Applicants characterized and demonstrated the potential of Cas8-HNH and Cas5-HNH nucleases for precise genome editing.

E. coli PAM Discovery Assay

100 ng each of one plasmid expressing the proteins and corresponding crRNA from the system of interest and one plasmid containing a target 8N degenerate flanking library plasmid were transformed by electroporation into 30 μL Endura Electrocompetent E. coli (Lucigen) as per the manufacturer's protocol, with 3 biological replicates per condition as well as 3 biological replicates of an empty control. After recovery by shaking at 37° C. for 1 hour, cells were plated across one 22.7 cm×22.7 cm BioAssay plate with the appropriate antibiotic resistance and grown for 12-16 h at 37° C. Cells were scraped from the plate and mixed well, and 2 mL of the scraped cells were used as input to minipreps (Qiagen). 100 ng miniprepped plasmids were input to PCR to amplify the PAM-containing region with an 8-cycle PCR using NEBNext High Fidelity 2× PCR Master Mix (NEB) with an annealing temperature of 63° C., followed by a second 12-cycle PCR to further add Illumina adaptors and barcodes. Amplified libraries were gel extracted, quantified by a Qubit dsDNA HS assay (Thermo Fisher Scientific) and subject to single-end sequencing on an Illumina NextSeq with Read 1 75 cycles, Index 1 8 cycles and Index 2 8 cycles. PAMs were extracted and Weblogos depicting PAMs depleted 5 standard deviations relative to the empty control were visualized using Weblogo3.

### Expression and Purification of Recombinant Proteins

Cas5-HNH, Cas8-HNH and DinG-HNH effector RNP complexes: To purify the Cas5-HNH, Cas8-HNH and DinG-HNH-associated effector complexes in complex with their cognate crRNAs, the E. coli codon optimized operons were cloned into pET45b(+) backbone. A His14 tag was inserted at the N-terminus of the Cas11 gene for Cas5-HNH, the Cas8-HNH gene for Cas8-HNH and the Cas5 gene in the DinG-HNH-associated Cascade by Gibson assembly. The crRNAs were similarly cloned into a separate pCOLADuet-1 vector under control of a T7 promoter, and co-expressed with the cognate Cascade proteins in BL21(DE3)pLysS strain (NEB). Cells were grown at 37° C. in autoinduction terrific broth (TB) medium (6) supplemented with 100 μg/ml ampicillin and 100 μg/ml kanamycin until reaching an optical density (OD600) of 0.4-0.6, then shifted to 18° C. for overnight induction. The bacteria were harvested by centrifugation and the cell paste was resuspended in lysis buffer (50 mM Tris-HCl pH 8, 200 mM NaCl, 5% glycerol, 40 mM imidazole and 5 mM β-mercaptoethanol) supplemented with protease inhibitors (PMSF and Roche complete, EDTA-free). The resuspended pellet was lysed by two passes with a high-pressure homogenizer (LM20 Microfluidizer, Microfluidics). The lysate was cleared by centrifugation, and the soluble fraction was mixed with Ni Sepharose 6 Fast Flow Affinity Chromatography Media (Cytiva) at 4° C. The resin was first washed with lysis buffer, then with 5 column volumes of buffer A (50 mM Tris-HCl pH 8, 1 M NaCl, 5% glycerol, 40 mM imidazole and 5 mM β-mercaptoethanol), and buffer B (50 mM Tris-HCl pH 8, 500 mM NaCl, 5% glycerol, 40 mM imidazole and 5 mM β-mercaptoethanol) on a gravity flow column. Bound RNP was then eluted in elution buffer (50 mM Tris-HCl pH 8, 500 mM NaCl, 5% glycerol, 300 mM imidazole and 5 mM β-mercaptoethanol). Eluted RNP was then dialyzed overnight into dialysis buffer (20 mM Tris-HCl pH 8, 250 mM NaCl, 5% glycerol and 1 mM DTT).

### Small RNA Sequencing

RNPs: Small RNA sequencing of RNPs was performed as previously described (7). Briefly, 300 μL of protein elute was mixed with 900 μL TRI reagent (Zymo) and incubated at room temperature for 5 min. 180 μL of chloroform (Sigma Aldrich) was added and the samples were mixed gently and incubated at room temperature for an additional 3 min, then spun at 12000×g for 15 min at 4° C. The aqueous phase was used as input for RNA extraction using a Direct-zol RNA miniprep plus kit (Zymo), with in-column DNAse treatment. The purified RNA was then subject to treatment as per manufacturer's instructions with 20 U of T4 PNK (NEB) for 1 hour at 37° C., then 20 U of RNA 5′ polyphosphatase (Biosearch Technologies) for 30 min at 37° C., with each enzymatic step followed by cleanup with a Zymo RNA Clean & Concentrator-5 kit as per the manufacturer's instructions. Following enzymatic treatments, purified RNA was subject to library preparation with an NEBNext Multiplex Small RNA Library Prep kit (NEB) as per the manufacturer's instructions, with an extension time of 1 min and 12 cycles in the final PCR. Amplified libraries were gel extracted, quantified by qPCR using a KAPA Library Quantification Kit for Illumina (Roche) on a CFX Opus 384 (BioRad) and sequenced on an Illumina MiSeq with Read 1 45 cycles, Read 2 45 cycles and Index 1 6 cycles. Adapters were trimmed using CutAdapt (8) and mapped to loci of interest using BWA. Filled reads were obtained and filled reads filtered as indicated were visualized using a custom Python script (7).

Heterologous loci in E. coli: Small RNA sequencing of heterologous loci in E. coli was performed as previously described (7). Briefly, Stb13 chemically competent E. coli were transformed with plasmids containing the locus of interest. A single colony was used to seed a 5 mL overnight culture. Following overnight growth, cultures were spun down, resuspended in 750 μL TRI reagent (Zymo) and incubated for 5 min at room temperature. 0.5 mm zirconia/silica beads (BioSpec Products) were added and the culture was vortexed for approximately 1 minute to mechanically lyse cells. 200 μL chloroform (Sigma Aldrich) was then added and the above protocol was followed with the following modifications: prior to T4 PNK treatment, ribosomal RNA was removed using an NEBNext rRNA depletion kit (Bacteria) (NEB).

Native expression: Organisms were obtained from NRRL or ATCC as freeze dried cultures, resuspended in the appropriate media and cultured as follows: Saccharamoronospora internatus (Agre et. al.) Greiner-Mai et. al. (ATCC 33517) was grown in ISP Medium 4 at 37° C. for 4 days and Amycolatopsis decaplanensis (NRRL B-24209) and Prauserella rugosa (NRRL B-2295) were grown in GYM Streptomyces media at 28° C. for 3 days. RNA was extracted and libraries were prepared as described for “Heterologous loci in E. coli” above.

### In Vitro Cleavage Assays

Cas5-HNH and Cas8-HNH dsDNA cleavage assays: DsDNA substrates were produced by PCR amplification of pUC19 plasmids containing the target sites and the TAM sequences. Cy3 and Cy5-conjugated DNA oligonucleotides (IDT) were used as primers to generate the labeled dsDNA substrates. Target cleavage assays contained 8 nM of DNA substrate, and RNP in a final 1× reaction buffer of 20 mM Tris pH 8, 50 mM NaCl and 5 mM MgCl₂. For the Cas8-HINH system, an RNP concentration of approximately 100 nM was used. For the Cas5-HNH system, RNP concentration could not be estimated due to super-stoichiometric levels of the tagged Cas11 protein in the sample. Assays were allowed to proceed at 37° C. for 1 hour. Reactions were then treated with 1.6 U of Proteinase K (NEB) at room temperature for 15 min, followed by treatment with 1 μL of 10 mg/mL RNAse A (Qiagen) for 5 min at room temperature. DNA was resolved by gel electrophoresis on 2% agarose E-gels (Thermo Fisher Scientific) as specified and imaged using a BioRad ChemiDoc imaging system.

E. coli Transformation Spot Assays

Stb13 competent cells were transformed with plasmids containing protein(s) of interest and corresponding crRNAs and selected on LB plates with 25 μM chloramphenicol and 50 μM kanamycin. Overnight cultures in TB were prepared then diluted 1:100 in selective ZymoBroth (Zymo Research) and cultured to an OD₆₀₀ of 0.6-0.8 at 37° C. at 220 rpm. Competent cells were prepared with a Mix & Go E. Coli transformation kit (Zymo Research) according to developer instructions. 20 μL of competent cells were transformed by heat shock with 100 ng pUC19 or plasmids containing a crRNA target and appropriate PAM and recovered in 80 μL of SOC media (Thermo Fisher Scientific) and for 2 hours at 37° C. at 220 rpm. Transformed cells were normalized to OD₆₀₀=0.1 then five 1:10 serial dilutions prepared in TB. 5 μL of each dilution were induced on LB plates with 25 μM chloramphenicol, 50 μM kanamycin, 100 μM ampicillin and 0.1 mM IPTG then incubated 24 hours at 37° C. Plates were imaged with a BioRad ChemiDoc imaging system.

### Sanger Sequencing of Cleavage Sites

In vitro cleavage assays were performed as described above and products were run on 2% agarose E-gels (Invitrogen) and cleaved products were extracted using a QIAquick gel extraction kit (Qiagen). Gel extracted cleavage products were submitted for Sanger sequencing by Genewiz/Azenta Life Sciences using primers 5′-AGTCACGACGTTGTAAAACGACGGCCAGTG-3′ (SEQ ID NO: 54) for the non-target strand and 5′-TCATTAGGCACCCCAGGCTTTAC-3′ (SEQ ID NO: 55) for the target strand.

### Mammalian Cell Culture and Transfection

Mammalian cell culture experiments were performed in the HEK293FT line (Thermo Fisher Scientific) grown in Dulbecco's Modified Eagle Medium with high glucose, sodium pyruvate, and GlutaMAX (Thermo Fisher Scientific), additionally supplemented with 1× penicillin-streptomycin (Thermo Fisher Scientific), 10 mM HEPES (Thermo Fisher Scientific), and 10% fetal bovine serum (VWR Seradigm). All cells were maintained at confluency below 80%.

Unless otherwise indicated, all transfections were performed with Lipofectamine 3000 (Thermo Fisher Scientific). Cells were plated 16-20 hours prior to transfection to ensure 90% confluency at the time of transfection. For 96-well plates, cells were plated at 20,000 cells/well. For each well on the plate, transfection plasmids were combined with 2 μL P3000 transfection enhancer (Thermo Fisher Scientific) per 1 μg DNA and OptiMEM I Reduced Serum Medium (Thermo Fisher Scientific) to a total of 25 μL. Separately, 23 μL of OptiMEM was combined with 2 μL of Lipofectamine 2000. Plasmid/P3000 and Lipofectamine solutions were then combined and pipetted onto cells.

For off-target genome editing, cells were plated in 12-well plates at 200,000 cells/well 16-20 hours prior to transfection to ensure 90% confluency at the time of transfection. To transfect, 200 ng of each protein expression plasmid and 500 ng of each crRNA expression plasmid per well were combined with OptiMEM I Reduced Serum Medium (Thermo Fisher Scientific) up to 250 μL. Separately, 5 μL of GeneJuice transfection reagent (EMD Millipore) was combined with 245 μL OptiMEM I Reduced Serum Medium (Thermo Fisher Scientific). DNA and GeneJuice mixes were combined to a final volume of 500 μL and pipetted onto cells.

### Mammalian Genome Editing

crRNA scaffold backbones were cloned into a pUC19-based human U6 expression backbone by Gibson Assembly. To clone guides, oligos with appropriate overhangs were synthesized by Genewiz, annealed and phosphorylated using T4 PNK (NEB) and cloned into crRNA backbones by restriction-ligation cloning. To clone protein expression constructs, E. coli codon optimized genes were cloned into a CMV expression backbone by Gibson assembly using 2× Gibson Assembly Master Mix (NEB) to generate pCMV-FLAG-SV40-Cas protein constructs.

50 ng each protein expression plasmid and 100 ng crRNA expression plasmid were transfected in each of 4 wells as biological replicates in a 96-well plate for each guide condition as described. An empty protein expression plasmid was used for dropout experiments as indicated. After 60-72 hours, genomic DNA was harvested by washing the cells once in 1×DPBS (Sigma Aldrich) and adding 50 μL QuickExtract DNA Extraction Solution (Lucigen). Cells were scraped from the plates to suspend in QuickExtract and cycled at 65° C. for 15 min, 68° C. for 15 min then 95° C. for 10 min to lyse cells. 2.5 μL of lysed cells were used as input into each PCR reaction. For library amplification, target genomic regions were amplified and with a 12-cycle PCR using NEBNext High Fidelity 2× PCR Master Mix (NEB) with an annealing temperature of 63° C. for 15 s, followed by a second 18-cycle round of PCR to add Illumina adapters and barcodes. The libraries were gel extracted and subject to single-end sequencing on an Illumina MiSeq with Read 1 300 cycles, Index 1 8 cycles, Index 2 8 cycles.

Insertion/deletion (indel) frequency was analyzed using CRISPResso2 (9). In order to eliminate noise from PCR and sequencing error, only indels with at least 2 reads or more than 1 base inserted or deleted were counted towards reported indel frequencies. To assess statistical significance, 2-tailed T-tests were performed using non-targeting guide/crRNA conditions as a negative control.

### Off-Target Genome Editing Analysis

Off-target genome editing was analyzed by tagmentation-based tag integration site sequencing (TTISS) (4). Briefly, donor oligos (5′-phospho-G*T*TGTGAGCAAGGGCGAGGAGGATAACGCCTCTCTCCCAGCGACT*A*T-3′ (SEQ ID NO: 56) and 5′-phospho-A*T*AGTCGCTGGGAGAGAGGCGTTATCCTCCTCGCCCTTGCTCACA*A*C-3′ (SEQ ID NO: 57), where * indicates a phosphorothioate modification) were annealed duplex buffer (30 mM HEPES pH 7.5, 100 mM KAc) at a final concentration of 10 μM per oligo by incubating in a thermocycler at 95° C. for 5 s, then ramping down to 4° C. at ˜0.1° C./s. Cells were transfected as described in “Mammalian cell culture and transfection” above. 72-96 hours after transfection, cells were harvested by washing each well with 1 mL PBS (Sigma Aldrich), then dry trypsinizing with 200 μL of TrypLE Express Enzyme (no phenol red) (Thermo Fisher Scientific) and resuspended in 1 mL PBS (Sigma Aldrich). Cell samples were then centrifuged at 300×g for 5 min at 4° C. to pellet and resuspended in 200 μL PBS (Sigma Aldrich). A DNeasy Blood & Tissue kit (Qiagen) was used to extract genomic DNA as per the manufacturer's instructions.

Genomic DNA was then tagmented by mixing lug DNA with Tn5 enzyme and TAPS buffer in a final volume of 100 μL and heating to 55° C. for 10 min. The tagmentation reactions were mixed with 500 μL of PB Binding buffer (Qiagen) and purified using a Qiaquick spin column (Qiagen). 10 μL of purified tagmented DNA was input into 2 rounds of PCR to add Illumina handles using 2× KOD Hot Start PCR master mix (EMD Millipore). The final libraries were gel extracted, quantified with a KAPA Library Quantification Kit (Roche), mixed with 10% PhiX Sequencing Control V3 (Illumina) and loaded on an Illumina NextSeq with the following read parameters: Read 1 59 cycles, Index 1 8 cycles, Read 2 25 cycles.

Off-target sites were identified by mapping reads to the hg38 assembly of the human genome using BrowserGenome.org as described (4). Identified sites were then filtered for those containing a continuous sequence with up to ⅓ of bases mismatched to the guide sequence and up to 1 mismatch in the PAM sequence to identify “true” off-target cleavage events.

### Size Exclusion Chromatography

Candidate Type VII Cas7/Cas5 complexes were purified as described, with the following modifications: buffer A contained 500 mM NaCl, buffer B contained 250 mM NaCl, and elution buffer contained 250 mM NaCl. The elution was directly diluted into to achieve a final concentration of 20 mM Tris-HCl pH 8, 150 mM NaCl, 100 mM imidazole and 3.6% glycerol. The sample was concentrated to 500 μL using a Vivaspin 20 column with a molecular weight cutoff of 50 kDa (Cytiva) and purified through a Superose 6 Increase 10/300 GL column (Cytiva) in 20 mM Tris-HCl pH 8, 150 mM NaCl, 1% glycerol. Fractions were collected and analyzed by SDS-PAGE gel electrophoresis.

### REFERENCES FOR EXAMPLE 2

1. S. Mulepati, S. Bailey, In vitro reconstitution of an Escherichia coli RNA-guided immune system reveals unidirectional, ATP-dependent degradation of DNA target. J. Biol. Chem. 288, 22184-22192 (2013).
2. M. L. Hochstrasser, D. W. Taylor, P. Bhat, C. K. Guegler, S. H. Sternberg, E. Nogales, J. A. Doudna, CasA mediates Cas3-catalyzed target degradation during CRISPR RNA-guided interference. Proc. Natl. Acad. Sci. U.S.A 111, 6618-6623 (2014).
3. R. E. Haurwitz, M. Jinek, B. Wiedenheft, K. Zhou, J. A. Doudna, Sequence- and structure-specific RNA processing by a CRISPR endonuclease. Science. 329, 1355-1358 (2010).
4. J. L. Schmid-Burgk, L. Gao, D. Li, Z. Gardner, J. Strecker, B. Lash, F. Zhang, Highly Parallel Profiling of Cas9 Variant Specificity. Mol. Cell. 78, 794-800.e8 (2020).
5. H. Altae-Tran, S. Kannan, F. E. Demircioglu, R. Oshiro, S. P. Nety, L. J. McKay, M. Dlakić, W. P. Inskeep, K. S. Makarova, R. K. Macrae, E. V. Koonin, F. Zhang, The widespread IS200/IS605 transposon family encodes diverse programmable RNA-guided endonucleases. Science. 374, 57-65 (2021).
6. S. Shmakov, O. O. Abudayyeh, K. S. Makarova, Y. I. Wolf, J. S. Gootenberg, E. Semenova, L. Minakhin, J. Joung, S. Konermann, K. Severinov, F. Zhang, E. V. Koonin, Discovery and functional characterization of diverse Class 2 CRISPR-Cas systems. Mol. Cell. 60, 385 (2015).
7 H. Altae-Tran, S. Kannan, F. E. Demircioglu, R. Oshiro, S. P. Nety, L. J. McKay, M. Dlakić, W. P. Inskeep, K. S. Makarova, R. K. Macrae, E. V. Koonin, F. Zhang, The widespread IS200/IS605 transposon family encodes diverse programmable RNA-guided endonucleases. Science. 374, 57-65 (2021).
8 K. S. Makarova, Y. I. Wolf, J. Iranzo, S. A. Shmakov, O. S. Alkhnbashi, S. J. J. Brouns, E. Charpentier, D. Cheng, D. H. Haft, P. Horvath, S. Moineau, F. J. M. Mojica, D. Scott, S. A. Shah, V. Siksnys, M. P. Terns, Č. Venclovas, M. F. White, A. F. Yakunin, W. Yan, F. Zhang, R. A. Garrett, R. Backofen, J. van der Oost, R. Barrangou, E. V. Koonin, Evolutionary classification of CRISPR-Cas systems: a burst of class 2 and derived variants. Nat. Rev. Microbiol. 18, 67-83 (2020).
9. S. Kannan, H. Altae-Tran, X. Jin, V. J. Madigan, R. Oshiro, K. S. Makarova, E. V. Koonin, F. Zhang, Compact RNA editors with small Cas13 proteins. Nat. Biotechnol. 40, 194-197 (2022).

TABLE 2
Guide sequences, data and statistical analysis related to genome editing experiments.

| | | | | | | | P-value |
|---|---|---|---|---|---|---|---|
| Guide/ | | | | | | | (two-tailed |
| site name | Guide sequence | Condition | indel_rep1 | indel_rep2 | indel_rep3 | indel_rep4 | T-test) |
| VEGFA | CCAGGCCCTCGTCATTGC | all proteins | 11.78457 | 11.14666 | 10.25015 | 10.70541 | 4.6667E−08 |
| guide 1 | AGCAGCCCCCGCAT | Cas5 | 0 | 0 | 0 | 0 | 0.355917684 |
| | (SEQ ID NO: 58) | -Cas6 | 0.116785 | 0.11248 | 0.080932 | 0.075848 | 0.000110354 |
| | | -Cas7 | 0 | 0 | 0.005426 | 0 | 0.961510749 |
| | | -Cas8-HNH | 0 | 0 | 0 | 0 | 0.355917684 |
| | | HNH KO | 0 | 0.009806 | 0 | 0 | 0.739074715 |
| | | NT guide | 0 | 0 | 0.005826 | 0 | |
| VEGFA | TTCTCTACCCACGAAGG | all proteins | 4.166985 | 4.974805 | 4.65866 | 5.016012 | 3.72389E−07 |
| guide 2 | GTTACGGCAAAGCAT | Cas5 | 0.097111 | 0.021594 | 0.030256 | 0.064082 | 0.889194288 |
| | (SEQ ID NO: 59) | -Cas6 | 0.042741 | 0.039001 | 0.046459 | 0.033154 | 0.532516249 |
| | | Cas7 | 0.046019 | 0.038177 | 0.030434 | 0.028458 | 0.375471283 |
| | | -Cas8-HNH | 0.047821 | 0.067379 | 0.054593 | 0.052166 | 0.725674867 |
| | | HNH KO | 0.032411 | 0.013207 | 0 | 0.031976 | 0.110140724 |
| | | NT guide | 0.021124 | 0.032027 | 0.063302 | 0.083556 | |
| CA2 | AGACCAATTGTCATGCTT | all proteins | 12.5962 | 13.63486 | 10.652 | 13.45837 | 1.65012E−06 |
| guide 1 | GACACAACTGCTGT | -Cas5 | 0.022176 | 0.006123 | 0.004549 | 0 | 0.140156234 |
| | (SEQ ID NO: 60) | -Cas6 | 0 | 0.020573 | 0.004522 | 0 | 0.246325084 |
| | | -Cas7 | 0 | 0.03096 | 0 | 0 | 0.355917684 |
| | | -Cas8-HNH | 0 | 0 | 0.010015 | 0.002721 | 0.226931037 |
| | | HNH KO | 0.007187 | 0.006237 | 0 | 0 | 0.135579629 |
| | | NT guide | 0 | 0 | 0 | 0 | |
| CA2 | AATTGTCATGCTTGACAC | all proteins | 3.608553 | 3.188037 | 3.445965 | 3.089125 | 1.35272E−07 |
| guide 2 | AACTGCTGTGGCTG | -Cas5 | 0 | 0.007844 | 0 | 0 | 0.355917684 |
| | (SEQ ID NO: 61) | -Cas6 | 0 | 0.028175 | 0.022543 | 0.017901 | 0.0305131 |
| | | -Cas7 | 0.005508 | 0 | 0 | 0.007938 | 0.144338071 |
| | | -Cas8-HNH | 0 | 0.009027 | 0 | 0.012555 | 0.142469245 |
| | | HNH KO | 0.003067 | 0 | 0.020345 | 0 | 0.275980665 |
| | | NT guide | 0 | 0 | 0 | 0 | |
| VEGFA | CCATCCTGTGTGCCCCTG | all proteins | 12.3611199 | 13.2771986 | 9.10875815 | 12.5860145 | 0.000288482 |
| guide 1 | ATGCGATGCGGGGG | -Cas5-HNH | 0.10805378 | 0.05165031 | 0.05708577 | 0.13220313 | 3.96067E−07 |
| | (SEQ ID NO: 62) | -Cas6 | 0.08435833 | 0.07266629 | 0.08457256 | 0.07021547 | 3.80333E−07 |
| | | -Cas7 | 0.15773686 | 0.17242441 | 0.24532159 | 0.18297314 | 4.52016E−07 |
| | | Cas8 | 0.04335744 | 0.09078863 | 0.1423938 | 0.03735618 | 3.97979E−07 |
| | | -Cas11 | 2.2162063 | 1.86402327 | 2.22272496 | 1.90306353 | 1.9165E−05 |
| | | HNH KO | 0.06678796 | 0.24770421 | 0.07535085 | 0.05567584 | 4.63927E−07 |
| | | NT guide | 0.0776969 | 0.04848794 | 0.05404756 | 0.13294926 | |
| VEGFA | ATGCCCACCTGCATGGT | all proteins | 8.91335227 | 8.82010063 | 9.73524261 | 9.23158571 | 8.75362E−09 |
| guide 2 | GATGTTGGACTCCTC | -Cas5-HNH | 0.0058713 | 0.01189167 | 0.01634895 | 0.03394501 | 0.298453826 |
| | (SEQ ID NO: 63) | -Cas6 | 0.00464501 | 0.00911868 | 0.0126717 | 0.02064031 | 0.568585377 |
| | | -Cas7 | 0.5758587 | 0.56096645 | 0.49931787 | 0.5560993 | 7.61083E−08 |
| | | Cas8 | 0 | 0.00459453 | 0.02030575 | 0.01374264 | 0.834549568 |
| | | Cas11 | 5.29626729 | 5.19144501 | 5.95238095 | 4.36585898 | 3.8501E−06 |
| | | HNH KO | 0.01076137 | 0.01029103 | 0.00543833 | 0.01482046 | 0.698234211 |
| | | NT guide | 0.08996483 | 0.06890392 | 0.06205461 | 0.13294926 | |
| DYNC1H1 | TCCTACATTAGAGAGTCT | all proteins | 7.52932065 | 9.17845321 | 7.60147233 | 8.57475984 | 8.53114E−07 |
| guide 1 | GGCAAGGCAGACAG | -Cas5-HNH | 0.03712814 | 0.05520404 | 0.12275178 | 0.06686139 | 0.033604135 |
| | (SEQ ID NO: 64) | -Cas6 | 0.06744138 | 0.07210626 | 0.05654219 | 0.09627287 | 0.001670091 |
| | | -Cas7 | 0.77186048 | 0.75827766 | 0.56430178 | 0.76234331 | 8.90983E−06 |
| | | -Cas8 | 0.09082895 | 0.08966965 | 0.03963262 | 0.05570676 | 0.010427021 |
| | | -Cas11 | 2.7560682 | 2.96806865 | 2.77179478 | 3.22474822 | 1.86907E−07 |
| | | HNH KO | 0.1970359 | 0.06510511 | 0.07317814 | 0.04056612 | 0.07413798 |
| | | NT guide | 0.04628884 | 0.17570281 | 0.07132813 | 0.08637443 | |
| DYNC1H1 | GAGCCACTGCATTGCTA | all proteins | 9.97750562 | 9.05583672 | 8.2864898 | 10.687546 | 2.07193E−06 |
| guide 2 | ATGAAAGAATGCAAA | -Cas5-HNH | 0.00406934 | 0 | 0 | 0.00851064 | 7.22015E−05 |
| | (SEQ ID NO: 65) | -Cas6 | 0 | 0 | 0 | 0 | 6.36252E−05 |
| | | -Cas7 | 0.84054156 | 0.67746408 | 0.79909597 | 1.00798346 | 0.000102703 |
| | | -Cas8 | 0.0096268 | 0.00808146 | 0.00174356 | 0.00566508 | 7.87928E−05 |
| | | -Cas11 | 2.51046025 | 3.44632176 | 3.56899349 | 3.25025351 | 1.50998E−05 |
| | | HNH KO | 0.00631772 | 0 | 0.00610044 | 0.00164902 | 7.21518E−05 |
| | | NT guide | 0.04226373 | 0.16315261 | 0.06521429 | 0.0780692 | |

TABLE 3
Redundant names for Cas proteins.

| CRISPR | | | |
|---|---|---|---|
| Type | Subtype | Cas name | Other names |
| V | V-A | Cas12a | Cpf1 |
| II | II | Cas9 | Cas5 (pre 2010, |
| | | | ambiguous old |
| | | | naming) |
| III | III-A/D | Cas7 | Csm3 |
| III | III-B/C | Cas7 | Cmr4 |
| III | III-E | Cas7 | gRAMP |
| IV | IV | Cas8-like | Csf1 |
| IV | IV | Cas7 | Csf2 |
| IV | IV | Cas5 | Csf3 |
| IV | IV | Cas6 | Csf5 |
| III | III-D | Cas5 | Csx10 |
| VI | II-A | Cas13a | c2c2 |

TABLE 4
List of plasmids used in this study and links to sequences.

| Plasmid | | Associated | |
|---|---|---|---|
| name | Description | figure(s) | Link to map |
| pHS1801 | JAGBLH010000220 Cas8-HNH full system (E. coli codon | 4C | SEQ ID 78 |
| | optimized) in pACYCDuet-1 with Lac promoters | | |
| pHS1928 | JAGBLH010000220 Cas8-HNH operon (E. coli codon optimized) | 4E, 4G, 4I | SEQ ID 79 |
| | with His-tagged Cas8 in pET45b(+) | | |
| pHS1929 | JAGBLH010000220 Cas8-HNH crRNA expression in | 4E, 4G, 4I | SEQ ID 80 |
| | pCOLADuet ™-1 | | |
| pHS1232 | MWEE01000013 Cas5-HNH operon (E. coli codon optimized) + | 4D | SEQ ID 81 |
| | CRISPR array in pACYCDuet-1 Lac promoters | | |
| pHS1477 | MWEE01000013 Cas5-HNH operon (E. coli codon optimized) | 4F, 4H, 4J | SEQ ID 82 |
| | with His14-tagged Cse2 in pET45b(+) | | |
| pHS1398 | MWEE01000013 Cas5-HNH crRNA expression in | 4F, 4H, 4J | SEQ ID 83 |
| | pCOLADuet ™-1 | | |
| pHS1859 | FLAG-SV40 NLS-Csy3/Cas7 from JAGBLH010000220 | 4K, 4M | SEQ ID 84 |
| | Cas8-HNH in pcDNA3.1(+) | | |
| pHS1860 | FLAG-SV40 NLS-Cas6/Csy4 from JAGBLH010000220 | 4K, 4M | SEQ ID 85 |
| | Cas8-HNH in pcDNA3.1(+) | | |
| pHS1861 | FLAG-SV40 NLS-Cas8-HNH from JAGBLH010000220 | 4K, 4M | SEQ ID 86 |
| | Cas8-HNH in pcDNA3.1(+) | | |
| pHS1862 | FLAG-SV40 NLS-Cas5 from JAGBLH010000220 | 4K, 4M | SEQ ID 87 |
| | Cas8-HNH in pcDNA3.1(+) | | |
| pHS1871 | U6-JAGBLH010000220 Cas8-HNH DR-BpiI-DR | 4K, 4M | SEQ ID 88 |
| pHS1514 | FLAG-SV40 NLS-Cas8/Cse1 from MWEE01000013 | 4L | SEQ ID 89 |
| | Cas5-HNH in pcDNA3.1(+) | | |
| pHS1515 | FLAG-SV40 NLS-Cas11/Cse2 from MWEE01000013 | 4L | SEQ ID 90 |
| | Cas5-HNH in pcDNA3.1(+) | | |
| pHS1516 | FLAG-SV40 NLS-Cas7 from MWEE01000013 Cas5-HNH | 4L | SEQ ID 91 |
| | in pcDNA3.1(+) | | |
| pHS1517 | FLAG-SV40 NLS-Cas5-HNH from MWEE01000013 | 4L | SEQ ID 92 |
| | Cas5-HNH in pcDNA3.1(+) | | |
| pHS1518 | FLAG-SV40 NLS-Cas6(Cse3/CasE) from MWEE01000013 | 4L | SEQ ID 93 |
| | Cas5-HNH in pcDNA3.1(+) | | |
| pHS1528 | U6-MWEE01000013 Cas5-HNH DR-BpiI-DR | 4L | SEQ ID 94 |

TABLE 5
Possible regions of an auto-regulatory loop that can be used to control activation
of HNH nuclease activity and/or interactions with other proteins in the complex.

| | | |
|---|---|---|
| Region | Sequence | Location |

Cas6 interactions with Cas5-HNH

| | | |
|---|---|---|
| 1 | QLGWVYGSKPEPKNVKVKEQGQGYWREHKYNPLRF | 193-230 |
| | RAA (SEQ ID NO: 95) | |
| 2 | SVLEPDWDYCFQNALDFLAAPPE (SEQ ID NO: 96) | 86-108 |
| 3 | LIDTGGNPDRPRPGRKWLDNIYNVHRRLSMAFPSGL | 8-65 |
| | RREQDPHFLKPFSPNDFQKTPF (SEQ ID NO: 97) | |

Cas11 interactions with Cas5-HNH

| | | |
|---|---|---|
| 4 | SLDIMQM (SEQ ID NO: 98) | 110-116 |
| 5 | SFWEQE (SEQ ID NO: 99) | 145-150 |
| 6 | RLDEKLPGFDLFSALWWPLRQKNQRAPKREV (SEQ ID NO: 100) | 30-60 |

Cas5-HNH interactions with the other two proteins:

| | | |
|---|---|---|
| 4 + 5 | TSVWRPKGT (SEQ ID NO: 101) | 266-274 |
| 6 | TRADYNNS (SEQ ID NO: 102) | 274-281 |
| 1 | NSEYKKVRAERLVMDHAACMVCKAPAT (SEQ ID NO: 103) | 280-306 |
| 2 + 3 | DHAACMVCKAPAT (SEQ ID NO: 104) | 294-306 |
| 2 + 3 | CRLCHDACTMLEYGS (SEQ ID NO: 105) | 329-343 |
| 6 | RLCHDACTMLEYGSGMTTNRI (SEQ ID NO: 106) | 330-360 |
| 2 + 3 | DPCDPIWRERILAKRKEIVEFRSRGQRFRKMKPEEEN | 351-388 |
| | G (SEQ ID NO: 107) | |
| 6 | PIWRERILAKRKEIVEFRSRGQRFRKMKPEEENG (SEQ | 355-388 |
| | ID NO: 108) | |

Reference Sequences
Cas5-HNH sequence:
MSAPPNTLFLRLEGALQSWGSNEAKFALRRTADAPTKSGVLGLLCAAMGIGRAEA
ADSWLPKLANLRMGVRIDRPGIRWWDFHTVGAGQRMRMAELKAPKKPSMVGA
ALAETLTPSKVKTRAETLLSRREYLADASFLVALQGEPELVAKLSAALAKPVWAI
YLGRKSCPPSRPVCEHPPGFYNTLEEALSAVPLQKRWHNEPLPQILPCVMDWIPGY
DGEHAPDDAEIHYDLPVSFQPPRHLPRFVIRRELVVGEDVQVSRETGTSVWRPKGT
RADYNNSEYKKVRAERLVMDHAACMVCKAPATTVQHVNYRRAGGKEIPEDLRA
LCRLCHDACTMLEYGSGMTTNRIDPCDPIWRERILAKRKEIVEFRSRGQRFRKMKP
EEENG (SEQ ID NO: 109)
Cas6 sequence:
MIYLSRLLIDTGGNPDRPRPGRKWLDNIYNVHRRLSMAFPSGLRREQDPHFLKPFS
PNDFQKTPFLFRVDNNIDGNDKRAIIIVQSVLEPDWDYCFQNALDFLAAPPETKEY
NPEFKAGQLLRFRLRVNASVRRHIPEMVQQDGQTIETGKILHKRVSLTWDASSTPD
QALADWLAAKSPKLGFTLQRCELLQLGWVYGSKPEPKNVKVKEQGQGYWREHK
YNPLRFRAALLEGVLEVDDPKLFLKTLSSGIGKAKSFGFGLLSVLPIRNDG (SEQ ID
NO: 110)
Cas11 sequence:
MNRGTVDFIASLENLKEGDLGILRKLRGARLDEKLPGFDLFSALWWPLRQKNQRA
PKREVAWLIAKLFAEFRFEQREGATLPILMGGICRKLEPKKELPRVLARFDQLASL
DIMQMEEPLSVIMGILRKHQQVCLDWVGLTDVLSFWEQEPVKREWSDSFIKAYKI
NKEDSDVD (SEQ ID NO: 111)

These regions (Table 5) may fight competitively to interact with the HINH domain and allow for conformational flexibility depending on the state of the cascade effector complex (bound to target or not). Regions 1-3 correspond to interactions with Cas6 while regions 4-6 correspond to interactions with Cas11.

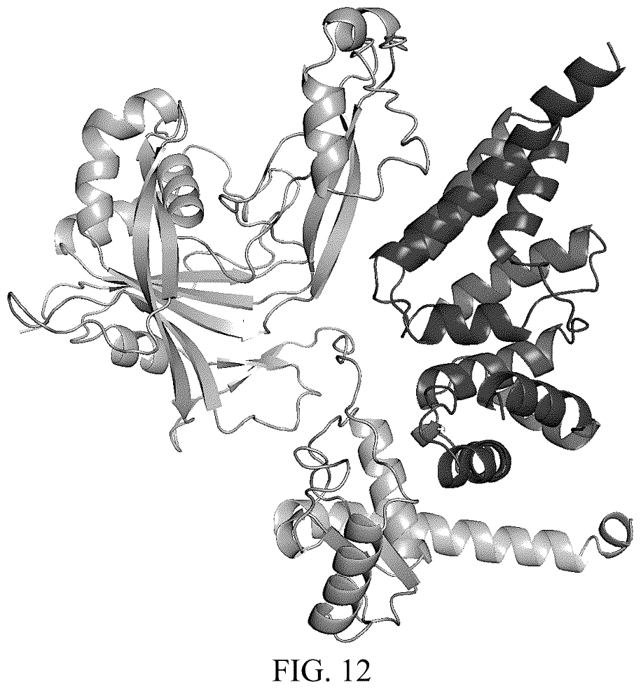

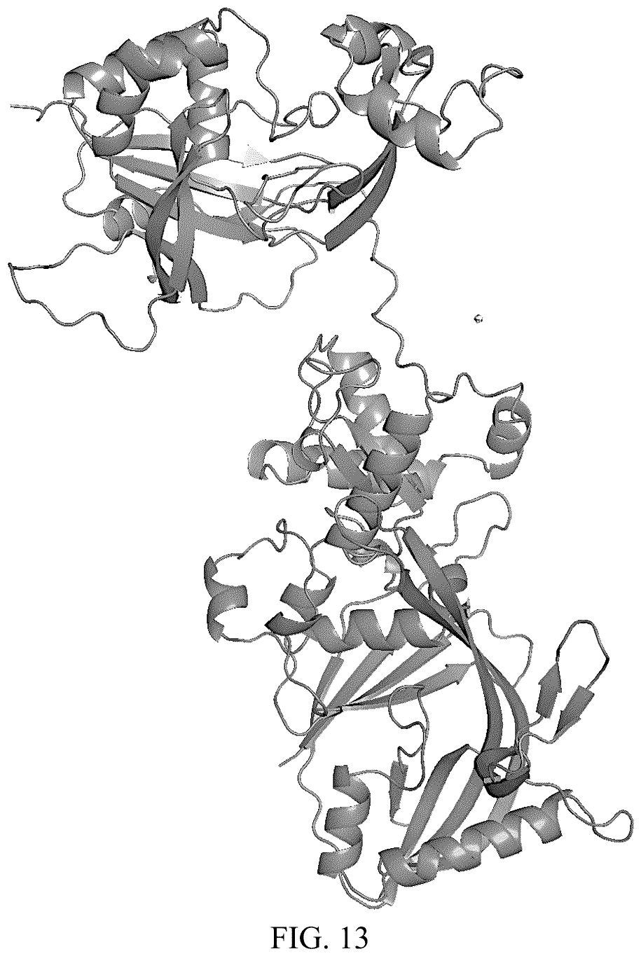

Alphafold structures demonstrating interactions for the corresponding proteins may be found in FIGS. 12 and 13.

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A non-naturally occurring, engineered composition comprising a) a hybrid CRISPR-Cas polypeptide comprising a Cas5 or Cas8 polypeptide, the Cas5 or Cas8 polypeptide comprising an HNH domain; and b) a guide RNA molecule comprising a reprogrammable spacer sequence, the guide RNA molecule capable of forming a complex with the hybrid CRISPR-Cas polypeptide and directing the polypeptide to a target polynucleotide.

2. The composition of claim 1, wherein the HNH domain is located in or at the C-terminus of the Cas5 or Cas8 polypeptide.

3. The composition of claim 1, wherein the composition comprises a Cas5 polypeptide, further comprising one or more of a Cas8e, a Cas11, a Cas7 and/or a Cas6 polypeptide or, wherein the composition comprises a Cas8 polypeptide, further comprising one or more of a Cas5, Cas6, Cas7, Cas11, and/or Cas12.

4. The composition of claim 1, wherein the hybrid CRISPR-Cas polypeptide comprises the amino acids corresponding to the about 1,000 to about 1,200 nucleic acids encoding the protein.

5. The composition of claim 1, wherein the reprogrammable spacer sequence comprises a spacer of 10 nucleotides to 40 nucleotides in length, preferably 15 to 35 nt in length.

6. The composition of claim 1, wherein the target sequence comprises a protospacer adjacent motif (PAM) sequence 5′ of the target polynucleotide, optionally wherein the PAM sequence comprises Ax, Tx, Gx, or xC wherein x consists of any nucleotide.

7. (canceled)

8. The composition of claim 1, wherein the target polynucleotide is DNA.

9. The composition of claim 1 wherein the guide RNA further comprises an aptamer.

10. The composition of claim 1 wherein the guide RNA molecule further comprises an extension to add an RNA template.

11. The composition of claim 1 claims, wherein the HNH domain of the hybrid CRISPR-Cas protein is catalytically inactive.

12. The composition of claim 1, further comprising a functional domain associated with the hybrid CRISPR-Cas protein, optionally wherein the functional domain has transposase activity, methylase activity, demethylase activity, translation activation activity, translation repression activity, transcription activation activity, transcription repression activity, translation release factor activity, chromatin modifying or remodeling activity, histone modification activity, nuclease activity, single-strand RNA cleavage activity, double-strand RNA cleavage activity, single-strand DNA cleavage activity, double-strand DNA cleavage activity, nucleic acid binding activity, detectable activity, or any combination thereof.

13. (canceled)

14. The composition of claim 12, comprising a serine or tyrosine recombinase, or a nucleotide deaminase.

15. (canceled)

16. The composition of claim 12, comprising a reverse transcriptase and further comprising a donor polynucleotide sequence, optionally wherein the donor polynucleotide further comprises a homology region to the target sequence on the 5′ end of the donor construct, the 3′ end of the donor construct, or both.

17. The composition of claim 12, comprising a non-LTR retrotransposon protein and further comprising a donor template encoding a donor polynucleotide sequence for insertion into the target polynucleotide and located between two binding elements capable of forming a complex with the non-LTR retrotransposon protein.

18. The composition of claim 12, comprising an integrase protein, and optionally a reverse transcriptase, and further comprising a donor template encoding a donor polynucleotide sequence for insertion into the target polynucleotide and located between two binding elements capable of forming a complex with the integrase protein.

19. The composition of claim 1, further comprising a homologous recombination donor template comprising a donor polynucleotide sequence for insertion into a target polynucleotide.

20. A vector system comprising one or more vectors encoding the hybrid CRISPR-Cas polypeptide and the guide RNA molecule of claim 1.

21. One or more polynucleotides encoding one or more components of the composition of claim 1.

22. One or more vectors encoding the one or more polynucleotides of claim 21.

23. An engineered cell comprising the composition of claim 1.

24. A cell or progeny thereof genetically engineered to express one or more components of the composition of claim 1.

25. A method of modifying a target polynucleotide sequence in a cell, comprising introducing to the cell the composition of claim 1, optionally wherein the hybrid CRISPR-Cas polypeptide cleaves at a PAM comprising Ax, Tx, Gx, or xC wherein x consists of any nucleotide.

26. (canceled)

27. The method of claim 25, wherein the polypeptide and/or nucleic acid components are provided via one or more polynucleotides encoding the polypeptides and/or nucleic acid component(s), and wherein the one or more polynucleotides are operably configured to express the CIRPSR-Cas hybrid polypeptide and/or the guide RNA molecule, optionally wherein the modifying comprises cleaving a DNA polynucleotide.

28. (canceled)

29. The method of claim 25, wherein contacting results in modification of a gene product or modification of the amount or expression of a gene product.

30. A method of editing nucleic acids in target polynucleotides comprising delivering the composition of claim 16 to a cell or population of cells comprising the target polynucleotides.

31. The method of claim 30, wherein the target polynucleotides are target sequences within genomic DNA, optionally wherein the target polynucleotide is edited at one or more bases to introduce a G→A or C→T mutation.

32. (canceled)

33. An isolated cell or progeny thereof comprising one or more base edits made using the method of claim 31.

34. A method of modifying target polynucleotides comprising; delivering the composition of claim 16 to a cell, or population of cells, comprising the target polynucleotide, wherein the complex directs the reverse transcriptase to the target sequence and the reverse transcriptase facilitates insertion of a donor sequence encoded by the donor template from the guide RNA molecule into the target polynucleotide, optionally wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

35. A method of modifying target polynucleotides comprising: delivering the composition of claim 17 to a cell, or population of cells, comprising the target polynucleotide, wherein the complex directs the non-LTR retrotransposon protein to the target sequence and the non-LTR retrotransposon protein facilitates insertion of the donor polynucleotide sequence from the donor construct into the target polynucleotide, optionally wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

36. A method of modifying target polynucleotides comprising: delivering the composition of claim 18 to a cell, or population of cells, comprising the target polynucleotide, wherein the complex directs the integrase protein to the target sequence and the integrase protein facilitates insertion of the donor polynucleotide sequence from the donor construct into the target polynucleotide, optionally wherein insertion of the donor sequence:
a. introduces one or more base edits;
b. corrects or introduces a premature stop codon;
c. disrupts a splice site;
d. inserts or restores a splice site;
e. inserts a gene or gene fragment at one or both alleles of the target polynucleotide; or;
f. a combination thereof.

37. (canceled)

38. An isolated cell or progeny thereof comprising the modifications made using the method of claim 34.

39. An isolated cell or progeny thereof comprising the modifications made using the method of claim 35.

40. An isolated cell or progeny thereof comprising the modifications made using the method of claim 36.
